(19) United States
(12) Patent Application Publication
(10) Pub. No.: US 2020/0270702 A1
(43) Pub. Date: Aug. 27, 2020

(54) CLASSIFICATION OF DIFFUSE LARGE B-CELL LYMPHOMA

(71) Applicants: TRUSTEES OF BOSTON UNIVERSITY, Boston, MA (US); DANA-FARBER CANCER INSTITUTE, INC., Boston, MA (US)

(72) Inventors: Stefano MONTI, Somerville, MA (US); Daniel GUSENLEITNER, Allston, MA (US); Margaret SHIPP, Wellesley, MA (US); Bjoern CHAPUY, Boston, MA (US)

(73) Assignees: TRUSTEES OF BOSTON UNIVERSITY, Boston, MA (US); DANA-FARBER CANCER INSTITUTE, INC., Boston, MA (US)

(21) Appl. No.: 16/471,994

(22) PCT Filed: Dec. 22, 2017
(86) PCT No.: PCT/US2017/068231

§ 371 Date: Jun. 20, 2019

Related U.S. Application Data
(60) Provisional application No. 62/438,761, filed on Dec. 23, 2016.

(51) Int. Cl. C12Q 1/6886

(52) U.S. Cl. CPC C12Q 1/6886; C12Q 2600/158

(57) ABSTRACT

Disclosed are methods and reagents for diagnosis, classification and treatment of DLBCL and subtypes thereof by means of gene expression profiling. Provided herein is a gene expression signature for use in obtaining diagnostic information for DLBCL and subtypes thereof. Aspects of the present disclosure relate to use of gene expression signature corresponding to particular subtype for classification of a sample from a subject and stratification of a subject for subtype-targeted clinical trial. Also provided herein is a computer based classification model for use in the methods disclosed herein.

### CROSS-REFERENCE TO RELATED APPLICATION

This application claims benefit under 35 U.S.C. § 119(e) of the U.S. Provisional Application No. 62/438,761 filed Dec. 23, 2016, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Dec. 21, 2017, is named 701586-086111-PCT_SL.txt and is 601,099 bytes in size.

### TECHNICAL FIELD

The present invention relates to methods and compositions for the diagnosis, classification and treatment of diffuse large B-cell lymphoma (DLBCL) and subtypes thereof.

### BACKGROUND

Diffuse large B-Cell lymphoma (DLBCL) is the most common type of non-Hodgkin's lymphoma in adults, accounting for ˜40% of B-cell malignancies. DLBCL is a clinically and genetically heterogeneous disease with recognized subtypes based on morphology, transcriptional profiles and multiple low-frequency genetic alterations including chromosomal translocations, somatic mutations and copy number alterations (CNA). Despite this recognized heterogeneity, and significant advances in the molecular and functional characterization of DLBCL, newly diagnosed patients are still largely treated with the same empiric regimen of Rituximab, Cyclophosphamide, Doxorubicin Hydrochloride, Vincristine Sulfate, Prednisone (the so called “R-CHOP” regimen). Although up to 60% of DLBCL patients are successfully treated with R-CHOP, the remainder have limited therapeutic options and often succumb to their disease (Friedberg, J. W. et al., 2008). Preclinical model systems that capture the genetic and functional heterogeneity of primary DLBCL are urgently needed.

The transcriptional heterogeneity of DLBCL is addressed by two classification schemes, cell of origin (COO) and consensus clustering classification (CCC) (Monti, S. et al., 2005; Wright, G. et al., 2003). Both of these systems highlight specific aspects of DLBCL biology, suggest cancer cell dependencies and identify rational therapeutic targets. The COO classification defines DLBCLs that share certain features with normal B-cell subtypes and includes “Germinal Center B-cell” (GCB)- and “Activated B-Cell” (ABC)-types (Basso, K. & Dalla-Favera, R, 2015; Lenz, G. & Staudt, L. M., 2010). More recently this classification was ported to the Nanostring platform using formalin-fixed paraffin-embedded tissue samples, and a reduced set of gene markers (Scott, D. W. et al., 2014).

Additional aspects of DLBCL functional heterogeneity are captured by the CCC system, which categorizes DLBCLs purely on the basis of the tumor transcriptome and defines “B-cell receptor” (BCR), “Oxidative Phosphorylation” (OxPhos) and “Host Response” (HR) tumor types (Monti, S. et al., 2005; Caro, P. et al., 2012; Chen, L. et al., 2013). The BCR-type DLBCLs have increased expression of proximal components of the B-cell receptor (BCR) pathway and increased reliance upon proximal BCR signaling and survival pathways (Monti, S. et al., 2005; Chen, L. et al., 2013; Chen, L. et al., 2008). BCR-independent OxPhos-DLBCLs exhibit enhanced mitochondrial energy transduction and selective reliance on fatty acid oxidation (Caro, P. et al., 2012). HR-type DLBCLs have a characteristic inflammatory/immune cell infiltrate and include the morphologically defined subset of T-cell/histiocyte-rich B-cell lymphomas (Monti, S. et al., 2005).

An ensemble classification scheme for the robust and accurate CCC classification of DLBCL samples profiled on transcriptome-wide Affymetrix expression microarrays (Polo et al. 2007) was previously developed. The ensemble classifier derived from well annotated frozen tissue samples was applied to the CCC prediction of DLBCL cell-lines and the predictions were functionally validated (Caro, P. et al., 2012; Chen, L. et al., 2013). However, there is an unmet need for reliable methods and compositions amenable for application to single samples in preclinical and clinical settings, for the diagnosis and classification of DLBCL into specific molecular subtypes that can guide treatment choices.

### SUMMARY

Provided herein are methods and reagents for diagnosis and classification of DLBCL into subtypes, as well as treatment guided by such classification. The methods, compositions and kits described herein are based in part on the discovery of a gene expression signature for diagnosing and classifying DLBCL. The gene signature comprises the expression levels of marker genes corresponding to SEQ ID NO: 1-141 or various subsets thereof for robust classification of a given sample or subject from whom the sample is derived, into one of three DLBCL subtypes: BCR-subtype; OxPhos-subtype; or HR-subtype. The inventors have developed a classifier which relies on the weighted expression levels of the 141 marker genes, or various subsets thereof, to provide accurate prediction of specific DLBCL subtype. Accordingly, the present disclosure encompasses a multivariate gene expression assay for diagnosing and classifying DLBCL. Also encompassed are subtype-targeted therapies for treatment of DLBCL.

Thus in one aspect, described herein is a method of treating a subject for DLBCL, the method comprising: (a) assaying a sample from a cancer cell of the subject, for levels of gene expression of genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE 1, ITPR3, SNRPA, PKMYT1, and SUPT5H and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB and at least three genes, at least four genes, at least five genes, at least six genes, at least seven genes, at least eight genes, at least nine genes, or ten genes selected from group consisting of SPCS3, SUCLG1, NDUFAB1, FADD, MRPS16, ATP6V1D, NDUFB1, NDUFB3, SEC11A and PARK7; (b) normalizing the assayed levels of gene expression with a control; (c) identifying the genes whose assayed levels of expression are upregulated; (d) administering a therapeutic regimen for the treatment of DLBCL of the BCR subtype if the expression of at least two genes, at least three genes, at least four genes, at least five genes, at least six genes, at least seven genes, at least eight genes or nine genes of the group consisting of TRMU, CKAP5, PLCG2, FUS, WEE1, ITPR3, SNRPA, PKMYT1, and SUPT5H are identified to be upregulated; or (e) administering a therapeutic regimen for the treatment of DLBCL of the Host Receptor subtype if the expression of at least two genes, at least three genes, at least four genes, at least five genes, at least six genes, at least seven genes, at least eight genes, at least nine genes, at least ten genes, at least eleven genes or twelve genes of the group consisting of PD-L1, CTLA4, IL15RA, GNS, PTPRM, AMICA, CFH, CD2, ITGAL, ACTN1, A2M and IL2RB are identified to be upregulated; or (f) administering a therapeutic regimen for the treatment of DLBCL of the OxPhos subtype if the expression of at least two genes, at least three genes, at least four genes, at least five genes, at least six genes, at least seven genes, at least eight genes, at least nine genes, or ten genes of the group consisting of SPCS3, SUCLG1, NDUFAB1, FADD, MRPS16, ATP6V1D, NDUFB1, NDUFB3, SEC11A and PARK7 are identified to be upregulated.

In one embodiment, the gene expression is assayed by measuring the nucleic acid encoded by the gene or by measuring or detecting a protein encoded by the gene. In some embodiments, the levels of gene expression are assayed by measuring the nucleic acid encoded by the gene using qPCR, microarray, or nCounter® analysis system. In some embodiments, the microarray is a cDNA array or an oligonucleotide array.

In some embodiments, the levels of gene expression are assayed by measuring or detecting a protein encoded by the gene using immunoassay, targeted mass spectrometry, or immunolabeling.

In some embodiments, step (c) is by linear combination of the normalized levels of gene expression obtained from step (b).

In some embodiments, the linear combination is a combination of weighted gene expression levels.

In some embodiments, step (c) comprises applying a classifier, wherein the classifier has been trained with training data from a plurality of DLBCL patients, wherein the training data comprise for each of the plurality of DLBCL patients (i) weighted gene expression level of at least the plurality of genes for which the expression levels are assayed including said at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes, or ten genes selected from group consisting of SPCS3, SUCLG1, NDUFAB1, FADD, MRPS16, ATP6V1D, NDUFB1, NDUFB3, SEC11A and PARK7 and (ii) information with respect to subtype of DLBCL based on the weighted gene expression level.

In some embodiments, the classifier is selected from Elastic net, Random Forest, and Shrunken centroids.

In some embodiments, the upregulation is relative to the levels of gene expression in a sample from a non-cancer cell.

In some embodiments, the subject is human.

In some embodiments, the DLBCL is a relapsed cancer. In some embodiments, the DLBCL is refractory to treatment with a CHOP or rituximab(R)/CHOP treatment regimen.

In some embodiments, the cancer cell is a cell obtained from tumor biopsy, frozen cancer tissue, or paraffin-embedded cancer tissue.

In some embodiments, the control in step c is the expression level of a housekeeping gene.

In some embodiments, the housekeeping gene is selected from genes in Table 4.

In another aspect, the technology described herein relates to a method for stratifying a subject for subtype-targeted therapy for DLBCL, the method comprising: (a) assaying a sample from a cancer cell of the subject, for levels of gene expression of genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB and at least three genes, at least four genes, at least five genes, at least six genes, at least seven genes, at least eight genes, at least nine genes, or ten genes selected from group consisting of SPCS3, SUCLG1, NDUFAB1, FADD, MRPS16, ATP6V1D, NDUFB1, NDUFB3, SEC11A and PARK7; (b) normalizing the assayed levels of gene expression with a control; (c) identifying the genes whose levels of expression are upregulated; (d) stratifying the subject for therapy comprising BCR subtype targeted therapy, if the expression of at least two genes, at least three genes, at least four genes, at least five genes, at least six genes, at least seven genes, at least eight genes or nine genes of the group consisting of TRMU, CKAP5, PLCG2, FUS, WEE1, ITPR3, SNRPA, PKMYT1, and SUPT5H are identified to be upregulated; or (e) stratifying the subject for therapy comprising Host Receptor subtype targeted therapy, if the expression of at least two genes, at least three genes, at least four genes, at least five genes, at least six genes, at least seven genes, at least eight genes, at least nine genes, at least ten genes, at least eleven genes or twelve genes of the group consisting of PD-L1, CTLA4, IL15RA, GNS, PTPRM, AMICA, CFH, CD2, ITGAL, ACTN1, A2M and IL2RB are identified to be upregulated; or (f) stratifying the subject for therapy comprising OxPhos subtype targeted therapy, if the expression of at least two genes, at least three genes, at least four genes, at least five genes, at least six genes, at least seven genes, at least eight genes, at least nine genes, at least ten genes of the group consisting of SPCS3, SUCLG1, NDUFAB1, FADD, MRPS16, ATP6V1D, NDUFB1, NDUFB3, SEC11A and PARK7 are identified to be upregulated.

In another aspect, the technology described herein relates to a method for diagnosing a DLBCL subtype in a subject having or suspected of having DLBCL, the method comprising: (a) assaying a sample from the subject, for levels of gene expression of genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB and at least three genes, at least four genes, at least five genes, at least six genes, at least seven genes, at least eight genes, at least nine genes, or ten genes selected from group consisting of SPCS3, SUCLG1, NDUFAB1, FADD, MRPS16, ATP6V1D, NDUFB1, NDUFB3, SEC11A and PARK7; (b) normalizing the assayed levels of gene expression with a control; (c) identifying the genes whose levels of expression are upregulated; (d) diagnosing the subject as suffering from DLBCL of the BCR-subtype, if the expression of at least two genes, at least three genes, at least four genes, at least five genes, at least six genes, at least seven genes, at least eight genes or nine genes of the group consisting of TRMU, CKAP5, PLCG2, FUS, WEE1, ITPR3, SNRPA, PKMYT1, and SUPT5H are identified to be upregulated; or (e) diagnosing the subject as suffering from DLBCL of the Host Receptor subtype, if the expression of at least two genes, at least three genes, at least four genes, at least five genes, at least six genes, at least seven genes, at least eight genes, at least nine genes, at least ten genes, at least eleven genes or twelve genes of the group consisting of PD-L1, CTLA4, IL15RA, GNS, PTPRM, AMICA, CFH, CD2, ITGAL, ACTN1, A2M and IL2RB are identified to be upregulated; or (f) diagnosing the subject as suffering from DLBCL of the OxPhos subtype, if the expression of at least two genes, at least three genes, at least four genes, at least five genes, at least six genes, at least seven genes, at least eight genes, at least nine genes or ten genes of the group consisting of SPCS3, SUCLG1, NDUFAB1, FADD, MRPS16, ATP6V1D, NDUFB1, NDUFB3, SEC11A and PARK7 are identified to be upregulated.

In another aspect, the technology described herein relates to a method of classifying a sample from a subject having or suspected of having DLBCL, the method comprising; (a) assaying a sample from the subject, for levels of gene expression of 141 genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE 1, ITPR3, SNRPA, PKMYT1, and SUPT5H and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB and at least three genes, at least four genes, at least five genes, at least six genes, at least seven genes, at least eight genes, at least nine genes, or ten genes selected from group consisting of SPCS3, SUCLG1, NDUFAB1, FADD, MRPS16, ATP6V1D, NDUFB1, NDUFB3, SEC11A and PARK7; (b) normalizing the assayed levels of gene expression with a control; (c) identifying the genes whose levels of expression are upregulated; (d) classifying the sample as corresponding to BCR-subtype of DLBCL, if the expression of at least two genes, at least three genes, at least four genes, at least five genes, at least six genes, at least seven genes, at least eight genes, at least nine genes of the group consisting of TRMU, CKAP5, PLCG2, FUS, WEE1, ITPR3, SNRPA, PKMYT1, and SUPT5H are identified to be upregulated; or (e) classifying the sample as corresponding to Host Receptor subtype of DLBCL, if the expression of at least two genes, at least three genes, at least four genes, at least five genes, at least six genes, at least seven genes, at least eight genes, at least nine genes, at least ten genes, at least eleven genes or twelve genes of the group consisting of PD-L1, CTLA4, IL15RA, GNS, PTPRM, AMICA, CFH, CD2, ITGAL, ACTN1, A2M and IL2RB are identified to be upregulated; or (f) classifying the sample as corresponding to OxPhos subtype of DLBCL, if the expression of at least two genes, at least three genes, at least four genes, at least five genes, at least six genes, at least seven genes, at least eight genes, at least nine genes or ten genes of the group consisting of SPCS3, SUCLG1, NDUFAB1, FADD, MRPS16, ATP6V1D, NDUFB1, NDUFB3, SEC 11A and PARK7 are identified to be upregulated.

In another aspect, described herein is a composition comprising an array comprising probes directed to genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE 1, ITPR3, SNRPA, PKMYT1, and SUPT5H, or at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, or at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In another aspect, described herein is a composition comprising an array consisting essentially of probes directed to genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE 1, ITPR3, SNRPA, PKMYT1, and SUPT5H, or at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, or at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In another aspect, described herein is a composition comprising an array comprising probes directed to genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE 1, ITPR3, SNRPA, PKMYT1, and SUPT5H and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In some embodiments, the array consists essentially of probes directed to genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In some embodiments, the array consists essentially of probes directed to genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, and at least two genes, at least three genes, at least four genes, at least five genes, at least six genes, at least seven genes, at least eight genes, at least nine genes, at least ten genes, at least eleven genes or twelve genes selected from the group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC 11A and PARK7. In some embodiments, the probes are cDNA probes.

In another aspect, the technology described herein relates to a kit comprising a plurality of probes for determining levels of gene expression of genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, or at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, or at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In another aspect, the technology described herein relates to a kit consisting essentially of a plurality of probes for determining levels of gene expression of genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, or at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, or at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In another aspect, the technology described herein relates to a kit comprising a plurality of probes for determining levels of gene expression of genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE 1, ITPR3, SNRPA, PKMYT1, and SUPT5H and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In some embodiments of the foregoing aspects, the kit consists essentially of a plurality of probes for determining levels of gene expression of genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In some embodiments, the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7.

In some embodiments, the probes are nucleic acid primers for amplification of the genes.

In some embodiments, each probe in the plurality of probes comprises a target specific sequence that hybridizes to no more than one gene under stringent hybridization conditions.

In some embodiments, the plurality of probes comprises probe pairs to detect the expression of genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE 1, ITPR3, SNRPA, PKMYT1, and SUPT5H and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7, wherein each probe in the probe pair comprises a target specific sequence that hybridizes to no more than one gene under stringent hybridization conditions, and wherein the target-specific sequences in each pair hybridize to different regions of the same gene.

In some embodiments, a probe molecule for each gene comprises a label.

In some embodiments, the probes are immobilized on a solid support.

In another aspect described herein is a kit for determining levels of gene expression of genes of SEQ ID NOs 1-141 or a subset thereof, the kit comprising antibodies or antigen binding portions thereof that specifically bind polypeptides encoded by the respective genes or subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, or at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, or at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7, wherein each antibody or antigen-binding portion thereof specifically binds to a protein expressed by one of the genes.

In another aspect, described herein is a kit for determining levels of gene expression of genes of SEQ ID NOs 1-141 or a subset thereof, the kit consisting essentially of antibodies or antigen binding portions thereof that specifically bind polypeptides encoded by the respective genes or subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, or at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, or at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7, wherein each antibody or antigen-binding portion thereof specifically binds to a protein expressed by one of the genes.

In another aspect, described herein is a kit for determining levels of gene expression of genes of SEQ ID NOs 1-141 or a subset thereof, the kit comprising antibodies or antigen binding portions thereof that specifically bind polypeptides encoded by the respective genes or subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7, wherein each antibody or antigen-binding portion thereof specifically binds to a protein expressed by one of the genes.

In some embodiments, the kit consists essentially of antibodies or antigen binding portions thereof that specifically bind respective polypeptides encoded by genes of SEQ ID NOs 1-141 or a subset thereof, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7, wherein each antibody or antigen-binding portion thereof specifically binds to a protein expressed by one of the genes.

In some embodiments, the kit consisting essentially of antibodies or antigen binding portions thereof that specifically bind respective polypeptides encoded by genes of SEQ ID NOs 1-141 or a subset thereof, wherein the subset comprises wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H, and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC 11A and PARK7, wherein each antibody or antigen-binding portion thereof specifically binds to a protein expressed by one of the genes.

In some embodiments, the antibodies or antigen binding fragments thereof are immobilized on a solid support.

In another aspect, described herein is a computer readable medium or computer program product comprising a classifier that predicts the DLBCL-subtype, based on weighted expression of genes of SEQ ID NOs 1-141 or a subset thereof in a sample from a subject having or suspected of having DLBCL, wherein the subset comprises at least four genes, at least five genes, at least six genes, at least seven genes, at least eight genes or nine genes selected from group consisting of TRMU, CKAP5, PLCG2, FUS, WEE1, ITPR3, SNRPA, PKMYT1, and SUPT5H and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7, said classifier having been trained by in silico analysis and classification algorithms.

In some embodiments, the classifier has been trained with training data from a plurality of DLBCL patients, wherein the training data comprise for each of the plurality of DLBCL patients (a) weighted gene expression level of at least the plurality of genes for which the expression levels are measured including said at least four genes, at least five genes, at least six genes, at least seven genes, at least eight genes or nine genes selected from group consisting of TRMU, CKAP5, PLCG2, FUS, WEE 1, ITPR3, SNRPA, PKMYT1, and SUPT5H, and at least two genes, at least three genes, at least four genes, at least five genes, at least six genes, at least seven genes, at least eight genes, at least nine genes, at least ten genes, at least eleven genes or twelve genes selected from group consisting of PD-L1, CTLA4, IL15RA, GNS, PTPRM, AMICA, CFH, CD2, ITGAL, ACTN1, A2M and IL2RB, and at least three genes, at least four genes, at least five genes, at least six genes, at least seven genes, at least eight genes, at least nine genes or ten genes selected from group consisting of SPCS3, SUCLG1, NDUFAB1, FADD, MRPS16, ATP6V1D, NDUFB1, NDUFB3, SEC11A and PARK7 and (b) information with respect to subtype of DLBCL based on the weighted gene expression level.

In some embodiments, the classifier is trained by one or more algorithms selected from the group consisting of dual ensemble, generalized simulated annealing, T-filter, CORG, CORG combined with support vector machine, dual bagging, single and pairs, forward learning, Laplacian based learning and learning method based on network perturbation amplitude.

In some embodiments, the classifier is trained with at least the data in the Gene Expression Omnibus datasets GSE2109, GSE 10245, GSE1 8842 and GSE37745.

In another aspect, the technology described herein relates to a method of treating a subject for Diffuse Large B Cell Lymphoma (DLBCL), the method comprising: (a) assaying a biological sample from a cancer cell of an individual with DLBCL for the expression of at least 15 of the genes corresponding to SEQ ID NOs 1-141; (b) normalizing the expression data for each of the assayed genes to a control; and (c) administering a therapeutic regimen for the treatment of DLBCL of the BCR subtype if the expression of at least two genes, at least three genes, at least four genes, at least five genes, at least six genes, at least seven genes, at least eight genes or nine genes of the group consisting of TRMU, CKAP5, PLCG2, FUS, WEE1, ITPR3, SNRPA, PKMYT1, and SUPT5H differs from that of the control; or (d) administering a therapeutic regimen for the treatment of DLBCL of the Host Receptor subtype if the expression of at least two genes, at least three genes, at least four genes, at least five genes, at least six genes, at least seven genes, at least eight genes, at least nine genes, at least ten genes, at least eleven genes or twelve genes of the group consisting of PD-L1, CTLA4, IL15RA, GNS, PTPRM, AMICA, CFH, CD2, ITGAL, ACTN1, A2M and IL2RB differs from that of the control; or (e) administering a therapeutic regimen for the treatment of DLBCL of the OxPhos subtype if the expression of at least two genes, at least three genes, at least four genes, at least five genes, at least six genes, at least seven genes, at least eight genes, at least nine genes or ten genes of the group consisting of SPCS3, SUCLG1, NDUFAB1, FADD, MRPS16, ATP6V1D, NDUFB1, NDUFB3, SEC11A and PARK7 differs from that of the control.

In another aspect, the technology described herein relates to a method of treating a subject for Diffuse Large B Cell Lymphoma (DLBCL), the method comprising: (a) assaying a biological sample from a cancer cell of an individual with DLBCL for the expression of at least 6 of the genes encoding TRMU, CKAP5, PLCG2, FUS, WEE1, ITPR3, SNRPA, PKMYT1, and SUPT5H; (b) normalizing the expression data for each of the assayed genes to a control; and (c) administering a therapeutic regimen for the treatment of DLBCL of the BCR subtype if the expression of at least two, at least three, at least four, at least five, at least six, at least seven, at least eight or nine of the genes encoding TRMU, CKAP5, PLCG2, FUS, WEE1, ITPR3, SNRPA, PKMYT1, and SUPT5H differs from that of the control; or (d) administering a therapeutic regimen for the treatment of DLBCL of the Host Receptor or OxPhos subtype if the expression of at least two, at least three, at least four, at least five, at least six, at least seven, at least eight or nine of the genes encoding TRMU, CKAP5, PLCG2, FUS, WEE1, ITPR3, SNRPA, PKMYT1, and SUPT5H does not differ from that of the control.

In another aspect, the technology described herein relates to a method of treating a subject for Diffuse Large B Cell Lymphoma (DLBCL), the method comprising: (a) assaying a biological sample from a cancer cell of an individual with DLBCL for the expression of at least two, at least three, at least four, at least five, at least six, at least seven, at least eight, at least nine, at least ten, at least eleven or twelve genes of the group consisting of PD-L1, CTLA4, IL15RA, GNS, PTPRM, AMICA, CFH, CD2, ITGAL, ACTN1, A2M and IL2RB; (b) normalizing the expression data for each of the assayed genes to a control; and (c) administering a therapeutic regimen for the treatment of DLBCL of the Host Receptor subtype if the expression of at least two, at least three, at least four, at least five, at least six, at least seven, at least eight, at least nine, at least ten, at least eleven or twelve of the genes encoding PD-L1, CTLA4, IL15RA, GNS, PTPRM, AMICA, CFH, CD2, ITGAL, ACTN1, A2M and IL2RB differs from that of the control; or (d) administering a therapeutic regimen for the treatment of DLBCL of the BCR or OxPhos subtype if the expression of at least two, at least three, at least four, at least five, at least six, at least seven, at least eight, at least nine, at least ten, at least eleven or twelve of the genes encoding PD-L1, CTLA4, IL15RA, GNS, PTPRM, AMICA, CFH, CD2, ITGAL, ACTN1, A2M and IL2RB does not differ from that of the control.

In another aspect, the technology described herein relates to a method of treating a subject for Diffuse Large B Cell Lymphoma (DLBCL), the method comprising: (a) assaying a biological sample from a cancer cell of an individual with DLBCL for the expression of at least three, at least four, at least five, at least six, at least seven, at least eight, at least nine or ten genes of the group consisting of SPCS3, SUCLG1, NDUFAB1, FADD, MRPS16, ATP6V1D, NDUFB1, NDUFB3, SEC 11A and PARK7; (b) normalizing the expression data for each of the assayed genes to a control; and (c) administering a therapeutic regimen for the treatment of DLBCL of the OxPhos subtype if the expression of at least two, at least three, at least four, at least five, at least six, at least seven, at least eight, at least nine or ten of the genes encoding SPCS3, SUCLG1, NDUFAB1, FADD, MRPS16, ATP6V1D, NDUFB1, NDUFB3, SEC11A and PARK7 differs from that of the control; or (d) administering a therapeutic regimen for the treatment of DLBCL of the BCR or Host Receptor subtype if the expression of at least two, at least three, at least four, at least five, at least six, at least seven, at least eight, at least nine or ten of the genes encoding SPCS3, SUCLG1, NDUFAB1, FADD, MRPS16, ATP6V1D, NDUFB1, NDUFB3, SEC11A and PARK7 does not differ from that of the control.

In another aspect, the technology described herein relates to a method of treating an individual for cancer, the method comprising: (a) assaying a biological sample from a cancer cell of an individual suspected having or having DLBCL for the expression of at least 15 of the genes corresponding to SEQ ID NOs 1-141; (b) normalizing the expression data for each of the assayed genes to a control; (c) administering a therapeutic agent for the treatment of DLBCL when the expression of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, least 21, at least 22, at least 23, at least 24, at least 25, at least 26, at least 27, at least 28, at least 29, at least 30 or at least 31 of the genes corresponding to SEQ ID NOs 1-141 differs from that of the control.

In some embodiments, the cancer is DLBCL.

In some embodiments, the control is a healthy control.

In some embodiments, the subject is human.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the term “marker”, refers to a gene or its gene product whose expression in a sample alone, or more often, in combination with one or more further genes, is characteristic of a disease or particular subtype of a disease (e.g., DLBCL or subtype thereof). The term can also refer to a product of gene expression, e.g., RNA transcribed from the gene or a translation product (i.e., a polypeptide) of such RNA, the production of which, alone or in combination with one or more further genes is characteristic of a disease or subtype thereof. In some cases expression of a marker gene may be the sole criterion used to define the disease or subtype thereof. The significance of the presence or absence of a marker gene expression product may vary depending upon the particular marker. In some cases the detection of a marker is highly specific in that it reflects a high probability that the disease is of a particular subtype. This specificity may come at the cost of sensitivity, i.e., a negative result may occur even if the sample is a sample that would be expected to express the marker. Conversely, markers with a high degree of sensitivity may be less specific than those with lower sensitivity. Thus it will be appreciated that a useful marker need not distinguish disease of a particular subtype with 100% accuracy. Furthermore, it will be appreciated that the use of multiple markers can improve the specificity and/or sensitivity with which a sample can be identified as being of a disease or a particular subtype thereof. The concept of a marker can be applied to individual cells, to tumors or to other disease states. In the case of tumors, a marker for a particular tumor class can include a gene whose expression is characteristic of a tumor or a particular subtype thereof, i.e., a gene whose expression is characteristic of some or all of the cells in the tumor. The term can also refer to a product of gene expression, e.g., an RNA transcribed from the gene or a translation product of such an RNA, the production of which is characteristic of a particular tumor type.

As used herein, “gene expression” refers to translation of information encoded in a gene into a gene product (e.g., RNA, protein). Expressed genes include genes that are transcribed into RNA (e.g., mRNA) that is subsequently translated into protein as well as genes that are transcribed into non-coding functional RNAs that are not translated into protein (e.g., miRNA, tRNA, rRNA, ribozymes etc.).

As used herein, “level of gene expression” or “expression level” refers to the level (e.g., amount) of one or more products (e.g. RNA, protein) encoded by a given gene in a sample or reference standard. The expression level can be relative or absolute.

The phrase “gene expression data” as used herein refers to information regarding the relative or absolute level of expression of a gene or set of genes in a cell or group of cells. “Gene expression data” can be acquired for an individual cell, or for a group of cells such as a tumor or biopsy sample.

The term “gene expression signature” or “signature” as used herein refers to a group of genes, the expression if which indicates a particular status of a cell, tissue, organ, organism or tumor. The genes making up this signature can be expressed, for example, in a specific cell lineage, stage of differentiation, during a particular biological response or in a disease or particular subtype thereof. The genes can reflect biological aspects of the tumors in which they are expressed, such as the cell of origin of a cancer, the nature of non-malignant cells in biopsy, or the oncogenic mechanisms responsible for the cancer.

As used herein “label” or “labeled” refers to a composition which is detectable by spectroscopic, photochemical, biochemical, immunochemical, electromagnetic, radiochemical, or chemical means. Non-limiting examples of useful labels include ³²P, fluorescent dyes, electron-dense reagents, enzymes (e.g., as commonly used in an ELISA), biotin, dioxigenin, or haptens and proteins or protein fragments (e.g., Myc, FLAG tag etc.) for which polyclonal or monoclonal antibodies are available.

A “labeled nucleic acid probe” is a nucleic acid probe that is bound, either covalently or through ionic, van der Waals or hydrogen bonds, directly or through a linker, to a label such that the presence of the probe can be detected by detecting the presence of the label bound to the probe.

As used herein “stringent hybridization conditions” refer to nucleic acid hybridization conditions that are generally selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. An extensive guide to the hybridization of nucleic acids is found in Tijssen (1993) Laboratory Techniques in Biochemistry and Molecular Biology-Hybridization with Nucleic Acid Probes Part I, Chapter 2 “Overview of principles of hybridization and the strategy of nucleic acid probe assays”, Elsevier, N.Y.

As used herein, “target nucleic acid” refers to a particular nucleic acid one wishes to detect. In one aspect, a target nucleic acid is a nucleic acid to which selective hybridization by a probe is desired.

As used herein, “subset” refers to a combination of genes or markers selected from a larger set of genes or markers, for example genes of SEQ ID NO. 1-141, the expression level of which in a sample is, for example, indicative of the specific subtype of DLBCL. Generally, a “subset” of a given group of genes or markers is at least one gene or marker fewer in number than the larger set of which it is a portion. In some embodiments, the expression level is relative to the expression of the gene in a sample obtained from a subject not suffering from cancer (e.g., healthy subject). In some embodiments, the expression level is relative to the other genes in a group or subset of genes or markers. In some embodiments, the subset of genes can be at least 90, at least 80, at least 70, at least 60, at least 50, at least 40, at least 30, at least 20, at least 15, at least 10 or at least 2 genes or markers or any combination thereof. In one embodiment, all 141 genes of Table 1 are assayed. In one embodiment, the subset comprises at least four genes selected from the group consisting of TRMU, CKAP5, PLCG2, FUS, WEE1, ITPR3, SNRPA, PKMYT1, and SUPT5H and at least two genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC 11A and PARK7.

The term “subtype”, (or simply “type”) also referred to herein as a tumor subset or tumor class, is a group of tumors that display one or more phenotypic or genotypic characteristics that distinguish members of the group from other tumors. When used in reference to DLBCL, the term “subtype” refers to one or more of the BCR, OxPhos and HR tumor phenotypes.

The term “diagnostic” as used herein refers to assays that provide results which can be used by one skilled in the art, sometimes in combination with results from other assays, to determine if an individual is suffering from a disease or disorder of interest such as DLBCL or particular subtype thereof. The term “prognostic” as used herein refers to the use of such assays to evaluate the response of an individual having such a disease or disorder to therapeutic or prophylactic treatment.

The term “refractory to (a treatment)” as used herein, means that a particular cancer either fails to respond favorably to a specific anti-neoplastic treatment, or alternatively, recurs or relapses after responding favorably to a specific anti-neoplastic treatment. For example, a DLBCL cancer “refractory to” CHOP or rituximab(R)/CHOP treatment means that a DLBCL either has failed to respond favorably to, or is resistant to, a treatment regimen that includes, CHOP or rituximab(R)/CHOP, or alternatively, has recurred or relapsed after responding favorably to such treatment regimen.

As used herein, the term “probe” refers to a nucleic acid, peptide or other chemical molecule or moiety (e.g., cDNA containing moiety) which specifically binds to a particular marker (e.g., genes or markers in Table 1 and Table 4). Probes are generally associated with or capable of associating with a detectable label. Typical labels include, for example, dyes, radioisotopes, luminescent and chemiluminescent moieties, fluorophores, enzymes, precipitating agents, amplification sequences, and the like. Probes can include proteins such as antigens derived from a marker, antibodies, nucleic acid molecules, carbohydrates, lipids, drugs, ions and any other compound that specifically binds to a marker contemplated for use with the methods and compositions described herein. In some embodiments, one or more probes are immobilized on a solid support, e.g., nitrocellulose, glass, nylon membrane, beads, particles and the like.

In one embodiment, the probe is a nucleic acid probe. A nucleic acid probe (e.g., a cDNA or nucleic acid primer) is capable of hybridizing specifically to a target nucleic acid of complementary sequence through complementary base pairing, via hydrogen bond formation. Accordingly, a probe can include natural (i.e., A, G, U, C, or T) or modified (7-deazaguanosine, inosine, etc.) bases. The bases in nucleic acid probes can be joined by a linkage other than a phosphodiester bond, so long as it does not interfere with hybridization. Thus, for example, probes can be peptide nucleic acids in which the constituent bases are joined by peptide bonds rather than phosphodiester linkages.

The phrase “hybridizing specifically to” or “specifically hybridizes” refers to the binding, duplexing, or hybridizing of a molecule only to a particular nucleotide sequence under stringent hybridization conditions when that sequence is present in a complex mixture (e.g., total cellular DNA or RNA).

As used herein, the term “oligonucleotide” refers to a short polymer composed of deoxyribonucleotides, ribonucleotides, or any combination thereof. Oligonucleotides are generally between about 10, 11, 12, 13, 14, 15, 20, 25, or 30 to about 150 nucleotides (nt) in length, more preferably about 10, 11, 12, 13, 14, 15, 20, 25, or 30 to about 70 nt, and most preferably between about 18 to about 26 nt in length.

As used herein, a “primer” is an oligonucleotide that is complementary to a target nucleotide sequence and leads to addition of nucleotides to the 3′ end of the target hybridized oligonucleotide in the presence of a DNA or RNA polymerase. The 3′ nucleotide of the primer should generally be complementary to the corresponding nucleotide of the target sequence when the primer hybridizes to the target sequence for optimal extension and/or amplification. As used herein, “amplification primers” refers to a pair of nucleic acid molecules that can anneal to 5′ or 3′ regions of a gene (plus and minus strands respectively or vice versa) and contain a defined region in between that can be amplified by repeated cycles of polymerization and primer annealing. The term “primer” includes all forms of primers that can be synthesized including peptide nucleic acid primers, locked nucleic acid primers, phosphorothioate modified primers, labeled primers, and the like. As used herein, a “forward primer” is a primer that is complementary to the anti-sense strand of dsDNA. A “reverse primer” is complementary to the sense-strand of dsDNA. In general, amplification primers are from about 10 to 30 nucleotides in length and flank a region from about 50 to 2000 nucleotides in length. Amplification primers can be used to produce a nucleic acid molecule comprising the nucleotide sequence flanked by the primers. A skilled artisan can readily determine appropriate primers (both nucleotide sequence and length) for amplifying and detecting the marker genes disclosed herein using art known methods and the nucleotide sequence of the marker genes.

An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. It is a specific, i.e., non-random, interaction between two complementary polynucleotides. Hybridization and the strength of hybridization (i.e., the strength of the association between the nucleic acids) is influenced by such factors as the degree of complementarity between the nucleic acids base composition, stringency of the conditions involved, and the Tm of the formed hybrid.

The term “antibody” as used herein is intended to include whole antibodies, e.g., of any isotype (IgG, IgA, IgM, IgE, etc), and includes fragments thereof which are also specifically reactive with target antigen. Antibodies can be fragmented using conventional techniques and the fragments screened for utility and/or interaction with a specific epitope of interest. Alternatively antigen fragments can be produced recombinantly. Thus, the term “antigen binding fragment” of an antibody includes proteolytically-cleaved or recombinantly-prepared portions of an antibody molecule that are capable of selectively reacting with a target antigen. Non-limiting examples of such proteolytic and/or recombinant fragments include Fab, F(ab′)2, Fab′, Fv, and single chain antibodies (scFv) containing a V_{L} and/or V_{H} domain joined by a peptide linker. The scFv's can be covalently or non-covalently linked to form antibodies having two or more binding sites. The term antibody also includes polyclonal, monoclonal, or other purified preparations of antibodies and recombinant antibodies.

“Array”, “microarray” or “chip” as used herein, refer to a plurality of nucleic acid (e.g., nucleic acid probes) or protein molecules (e.g., antibodies or fragments thereof) arranged at known positions on a solid support (e.g., a glass slide, a bead, a nitrocellulose membrane, or in a well of a microtitre plate or gels). An array can include a plurality of molecules of a single class (e.g., polynucleotides) or a mixture of different classes (e.g., an array including both proteins and nucleic acids immobilized on a single substrate). Microarrays have been generally described in the art in, for example, U.S. Pat. No. 5,143,854 (Pirrung), U.S. Pat. No. 5,424,186 (Fodor), U.S. Pat. No. 5,445,934 (Fodor), U.S. Pat. No. 5,677,195 (Winkler), U.S. Pat. No. 5,744,305 (Fodor), U.S. Pat. No. 5,800,992 (Fodor), U.S. Pat. No. 6,040,193 (Winkler), and Fodor et al. 1991. Light-directed, spatially addressable parallel chemical synthesis. Science, 251:767-777. Each of these references is incorporated by reference herein in their entirety.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. In some aspects the probes of an array are completely complementary to the target sequence over the length of the probe. A 25 base probe, for example would be complementary over that 25 bases to a contiguous 25 bases of the target. The target can be longer than 25 bases.

“Multivariate dataset” as used herein, refers to any dataset comprising a plurality of different variables including, but not limited, to chemogenomic datasets comprising log ratios from differential gene expression experiments, such as those carried out on polynucleotide microarrays, or multiple protein binding affinities measured using a protein chip. Other examples of multivariate data include assemblies of data from a plurality of standard toxicological or pharmacological assays (e.g., blood analytes measured using enzymatic assays, antibody based ELISA or other detection techniques). “Variable” as used herein, refers to any value that may vary. For example, variables may include relative or absolute amounts of biological molecules, such as mRNA or proteins, or other biological metabolites. A multivariate dataset can comprise for example, expression levels of a plurality of genes.

“Classifier” as used herein, refers to a function of a set of variables that is capable of answering a classification question. A “classification question” can be of any type susceptible to yielding a yes or no answer (e.g., “Is the unknown a member of the class or does it belong with everything else outside the class?”). For example, a classifier as it relates to the instant disclosure is capable of answering the classification question, if a given sample from a subject represents a particular subtype of DLBCL. The set of variables, based on which the classifier can answer this classification question, can be gene expression level of one or more marker genes disclosed in Table 1. The classifier can be a “Linear classifier”, comprising a first order function of a set of variables, for example, a summation of a weighted set of gene expression log ratios. A valid classifier is defined as a classifier capable of achieving a performance for its classification task at or above a selected threshold value. For example, a log odds ratio >4.00 represents a preferred threshold of the present disclosure. Higher or lower threshold values can be selected depending of the specific classification task. A classifier can use a combination of variables, weighting factors, and other constants that provide a unique value or function capable of answering a classification question. “Weighting factor” (or “weight”) as used herein, refers to a value used by an algorithm in combination with a variable in order to adjust the contribution of the variable.

The term “normalization” means to convert a numerical value, such as fluorescence intensity, which has been obtained in a gene expression analysis or the like, into a numerical value that permits a comparison with all measurement values obtained by other gene expression analyses. In order to minimize expression measurement variations due to non-biological variations in samples, e.g., the amount and quality of expression product to be measured, raw expression level data measured for a gene product (e.g., cycle threshold (Ct) measurements obtained by qRT-PCR) can be normalized relative to the mean expression level data obtained for one or more control. The term “control” or “normalizing factor” are used interchangeably and refer to expression of mRNA or protein of a reference gene against which the amounts of marker or combination of markers of interest are normalized to permit comparison of amounts of the mRNA or protein of interest among different biological samples. Typically a “reference gene”, is constitutively expressed and is not differentially regulated between at least two physiological states or conditions from which samples will be analyzed, e.g., given disease and non-disease states. Thus, for example, a normalizing control does not vary substantially outside of a range found in a normal healthy population (e.g., <30%, <25%, <20%, <15%, <10%, <7%, <5%, <4%, <3%, <2%, <1% or less) or in the presence and absence of e.g., cancer. In one approach to normalization, a small number of genes are used as reference genes; the genes chosen for reference genes typically show a minimal amount of variation in expression from sample to sample and the expression level of other genes is compared to the relatively stable expression of the reference genes. In a global normalization approach, the expression level of each gene in a sample is compared to an average expression level in the sample of all genes in order to compare the expression of a particular gene to the total amount of material. In some embodiments, the reference gene is a “housekeeping gene”.

As used herein, the term “housekeeping gene” refers to a gene encoding a transcript and/or protein that is constitutively expressed, and is necessary for basic maintenance and essential cellular functions. A housekeeping gene generally is not expressed in a cell- or tissue-dependent manner, most often being expressed by all cells in a given organism. Some examples of housekeeping proteins include HMBS, actin, tubulin, GAPDH, among others. In some embodiments, the housekeeping genes are one or more genes selected from Table 4.

As used herein, the phrase “normalized to the expression level of a housekeeping gene” or “normalizing” refers to the conversion of a data value representing the expression level of one or more genes in a sample by dividing it by the expression data value representing the level of a normalizing control (e.g., housekeeping genes selected from Table 4), thereby permitting comparison of normalized marker values among a plurality of samples or to a reference. In some embodiments, the normalizing can be carried out as set forth in the Examples herein.

The term “statistically significant” or “significantly” refers to statistical significance and generally means two standard deviations (2SD) or more above or below normal or a reference. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

The term “in need thereof” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

As used herein, the term “administering,” refers to the placement of a compound as disclosed herein into a subject by a method or route that results in at least partial delivery of the compound at a desired site. Pharmaceutical compositions comprising the compounds disclosed herein can be administered by any appropriate route which results in an effective treatment in the subject, e.g., topical administration, oral administration, intravenous administration, intraperitoneal administration, subcutaneous administration, intramuscular administration, intracerebroventricular (“icv”) administration, intranasal administration, intracranial administration, intracelial administration, intracerebellar administration, or intrathecal administration.

As used herein, a “subject”, “patient”, “individual” and like terms are used interchangeably and refers to a vertebrate, preferably a mammal, more preferably a primate, still more preferably a human. Mammals include, without limitation, humans, primates, rodents, wild or domesticated animals, including feral animals, farm animals, sport animals, and pets. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The terms, “individual,” “patient” and “subject” are used interchangeably herein. A subject can be male or female.

Preferably, the subject is a mammal. The mammal can be a human, non-human primate, mouse, rat, dog, cat, horse, or cow, but is not limited to these examples. Mammals other than humans can be advantageously used as subjects that represent animal models of conditions or disorders associated with cancer (e.g., DLBCL or subtypes thereof). In addition, the compositions and methods described herein can be used to treat domesticated animals and/or pets.

A subject can be one who has been previously diagnosed with or identified as suffering from or under medical supervision for a cancer (e.g., DLBCL). A subject can be one who is diagnosed and currently being treated for, or seeking treatment, monitoring, adjustment or modification of an existing therapeutic treatment, or is at a risk of developing cancer such as DLBCL, e.g., due to family history etc.

As used herein, the terms “protein”, “peptide” and “polypeptide” are used to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically acceptable carrier” means a pharmaceutically-acceptable material, composition or vehicle, such as a liquid or solid filler, diluent, excipient, manufacturing aid or solvent encapsulating material necessary or used in formulating an active ingredient or agent for delivery to a subject. Each carrier must be “acceptable” in the sense of being compatible with the other ingredients of the formulation and not injurious to the patient.

The terms “upregulation” “increased”, “increase”, or “enhance” are all used herein to generally mean an increase by a statically significant amount; for the avoidance of doubt, the terms “increased”, “increase”, or “enhance”, mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, the term “upregulation” refers to genes comprising higher weights relative to the other genes assayed in a gene set. Methods for determining weighted gene expression are described below.

The terms, “decrease”, “reduce”, “reduction”, “lower” or “lowering,” or “inhibit” are all used herein generally to mean a decrease by a statistically significant amount. For example, “decrease”, “reduce”, “reduction”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

Nucleic acid sequences for the genes of SEQ ID NO: 1-141 listed in Table 1 are available under the accession numbers listed in Table 2. The respective sequences are incorporated herein by reference to their respective accession numbers.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the methods and compositions described herein can be made and used using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other than in the operating examples, or where otherwise indicated, all numbers expressing quantities of ingredients or reaction conditions used herein should be understood as modified in all instances by the term “about.” The term “about” when used in connection with percentages means±1% of the value being referred to. For example, about 100 means from 99 to 101.

Although methods and materials similar or equivalent to those described herein can be used in the practice or testing of this disclosure, suitable methods and materials are described below. The abbreviation, “e.g.,” is derived from the Latin exempli gratia, and is used herein to indicate a non-limiting example. Thus, the abbreviation “e.g.,” is synonymous with the term “for example.”

As used in this specification and appended claims, the singular forms “a,” “an”, and “the” include plural references unless the context clearly dictates otherwise. Thus for example, reference to “the method” included one or more methods, and/or steps of the type described herein and/or which will become apparent to those persons skilled in the art upon reading this disclosure and so forth.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

### BRIEF DESCRIPTION OF THE DRAWINGS

FIG. 1 shows overview of the workflow. The different cohorts used are shown herein, on top are all the Affymetrix based datasets, on the bottom are the Nanostring data. As gold standard labels, the ensemble classifier CCC prediction described in reference, Polo, J. M. et al., 2007; Monti, S. et al., 2012 was used. Based on Discovery Set I a multinominal leastic net classification model was trained that is able to predict CCC classes based on 142 genes. This parsimonious classifier was applied to all datasets and the results were compared to the ensemble classifier results. Of note is that the three validation sets (one Affymetrix and both Nanostring sets) are based on the same 44 tumor biopsy samples, which have been processed and assayed in a different manner; hence the gold standard labels were derived on the Affymetrix set and then used in the Nanostring sets.

FIG. 2 shows CCC Heatmap for Nanostring fresh frozen dataset. The samples are ordered by class probabilities, based on the predictions of an leastic net model trained on the discovery set I. The top barplot shows the single class probabilities of the classifier, the color-bars below show the gold standard and predicted CCC subgroups. Each row corresponds to a gene in our CCC signature, which are grouped by class and weights within the leastic net model. Only the top 15 genes for each class are shown.

FIG. 3 shows CCC learning curves for the 44 sample Nanostring frozen dataset. Here is shown how well classification works depending on differing sample sizes. For each increment, classification was rerun 50 times based on random sampled subsets. The line shows the trend of classification performance, while the error bars show the 95% confidence intervals based on the 50 reruns. There is a significant upward trend indicating that a larger sample size would result in a better classification performance.

FIG. 4 shows heatmap curve of the 44 replicates of the validation cohort in Affymetrix. The samples are ordered by class probabilities, based on the predictions of an leastic net model trained on the discovery set I. The top barplot shows the single class probabilities of the classifier, the bars below show the gold standard and predicted CCC subgroups. Each row corresponds to a gene in our CCC signature, which are grouped by class and weights within the leastic net model.

FIG. 5 shows heatmap curve of the 44 replicates of the validation cohort in Nanostring FFPE. The samples are ordered by class probabilities, based on the predictions of an leastic net model trained on the Discovery Set I. The top barplot shows the single class probabilities of the classifier, the color-bars below show the gold standard and predicted CCC subgroups. Each row corresponds to a gene in the CCC signature, which are grouped by class and weights within the leastic net model.

FIG. 6 shows Nanostring frozen leave-one-out cross-validation (LOOCV). The samples are ordered by class probabilities, based on the predictions of an leastic net model trained on the discovery set I. The top barplot shows the single class probabilities of the classifier, the color-bars below show the gold standard and predicted CCC subgroups. Each row corresponds to a gene in our CCC signature, which are grouped by class and weights within the leastic net model.

FIG. 7 shows Nanostring FFPE LOOCV. The samples are ordered by class probabilities, based on the predictions of an leastic net model trained on Discovery Set I. The top barplot shows the single class probabilities of the classifier, the color-bars below show the gold standard and predicted CCC subgroups. Each row corresponds to a gene in our CCC signature, which are grouped by class and weights within the leastic net model.

FIG. 8 shows learning curves for the 44 sample Nanostring FFPE dataset. This example shows how well classification works depending on differing sample sizes. For each increment, classification was rerun 50 times based on random sampled subsets. The line shows the trend of classification performance, while the error bars show the 95% confidence intervals based on the 50 reruns. There is a slight upward trend indicating that a larger sample size would result in a better classification performance.

FIG. 9 shows CCC heatmap for Nanostring fresh frozen dataset. The samples are ordered by class probabilities, based on the predictions of an leastic net model trained on the discovery set I. The top barplot shows the single class probabilities of the classifier, the color-bars below shows the gold standard and predicted CCC subgroups. Each row corresponds to a gene in our CCC signature, which are grouped by class and weights within the leastic net model.

FIGs. 10A and 10B shows within vs. across correlation between Affymetrix and Nanostring Frozen/FFPE. The two plots show histograms of the across correlations between different genes on different platforms in red and within correlations (between the same genes on different platforms) in blue. The across correlations are centered at 0. FIG. 10A shows the correlations between Affymetrix and Nanostring frozen, whereas FIG. 10B shows the correlations between Affymetrix and Nanostring FFPE.

### DETAILED DESCRIPTION

Provided herein are methods and reagents for diagnosis and classification of DLBCL into subtypes. The methods, compositions and kits described herein are based in part on the discovery of a gene expression signature for diagnosing and classifying DLBCL. The gene expression signature comprises the expression levels of marker genes corresponding to SEQ ID NO: 1-141 or various subsets thereof, for robust classification of a given sample or subject from whom the sample is derived, into one of three Diffuse large B-cell lymphoma (DLBCL) subtypes: BCR-subtype; OxPhos-subtype; or HR-subtype. The inventors have developed a classifier which relies on the weighted expression levels of the 141 marker genes or various subsets thereof to provide accurate determination of specific DLBCL subtype. Accordingly, the present disclosure encompasses a multivariate gene expression assay for diagnosing and classifying DLBCL, followed by subtype-targeted therapy for treatment of DLBCL.

### DLBCL and Sub-Types Thereof

The methods and compositions described herein allow diagnosis of specific DLBCL subtype. In one aspect, then, the methods and compositions permit treatment of DLBCL with a therapeutic regimen that is specific for the diagnosed DLBCL subtype.

Diffuse large B-cell lymphoma is a fast-growing, aggressive form of non-Hodgkin's lymphoma (NHL) which originates in centrocytes in the light zone of germinal centers. It is one of the most common types of NHL. Several types of DLBCL are known in the art, based on pathological studies, clinical staging procedures and transcriptional analyses.

The transcriptional heterogeneity of DLBCL is generally addressed, for example, by classification schemes referred to as the cell of origin (COO) and consensus clustering classification (CCC) schemes (Monti, S. et al., 2005; Wright, G. et al., 2003). Both of these systems highlight specific aspects of DLBCL biology, suggest cancer cell dependencies and identify rational therapeutic targets. The COO classification defines DLBCLs that share certain features with normal B-cell subtypes and includes “Germinal Center B-cell” (GCB)- and “Activated B-Cell” (ABC)-types (Basso, K. & Dalla-Favera, R, 2015; Lenz, G. & Staudt, L. M., 2010). Additional aspects of DLBCL functional heterogeneity are captured by the CCC system, which categorizes DLBCLs purely on the basis of the tumor transcriptome and defines “B-cell receptor” (BCR), “Oxidative Phosphorylation” (OxPhos) and “Host Response” (HR) tumor subtypes (Monti, S. et al., 2005; Caro, P. et al., 2012; Chen, L. et al., 2013). The methods and compositions described herein permit the accurate classification of a DLBCL into one of the BCR, OxPhos or HR subtypes on the basis of the expression of a limited set of signature genes for each subtype.

The “B-cell receptor (BCR)-type DLBCL” or “BCR subtype” refers to a subclass of DLBCL having reliance upon proximal BCR signaling and survival pathways (Monti, S. et al., 2005; Chen, L. et al., 2013; Chen, L. et al., 2008). BCR-DLBCLs tend to exhibit additional genetic alterations of proximal BCR pathway components including copy gain of SYK and copy loss of PI3K negative regulator, PTEN (Chen, L. et al., 2013). As it relates to the instant disclosure, the “BCR subtype” can also refer to DLBCL that display upregulation in one or more genes selected from the group: TRMU; CKAP5; PLCG2; FUS; WEE1; ITPR3; SNRPA; PKMYT1 and SUPT5H. DLBCL of the BCR subtype tend to respond better to therapy with inhibitors of the BCR signaling and survival pathways. These tumors include BCR-dependent ABC-type DLBCLs with high baseline NF-κB activity and BCR-dependent GCB-type DLBCLs with low baseline NF-κB activity that largely rely upon SYK/PI3K/AKT signaling (Chen, L. et al., 2013). The consequences of proximal BCR/SYK/PI3K pathway inhibition differ in BCR-dependent DLBCLs with high vs. low baseline NF-κB activity (ABC- or GCB-types, respectively). In BCR/ABC-type DLBCLs, proximal BCR pathway inhibition decreases the abundance of NF-κB target genes including anti-apoptotic BCL2 family members such as BFL1/A1 (Chen, L. et al., 2013). In contrast, proximal BCR pathway inhibition induces the pro-apoptotic BH3 family member, HRK, in BCR/GCB-type DLBCLs. BCR-DLBCLs exhibit additional genetic alterations of proximal BCR pathway components including copy gain of SYK and copy loss of PI3K negative regulator, PTEN (Chen, L. et al., 2013).

The “OxPhos-type DLBCL” or “OxPhos-subtype” refers to a subclass of DLBCL that exhibit enhanced mitochondrial energy transduction and selective reliance on fatty acid oxidation (Caro, P. et al., 2012). The OxPhos-subtype is characterized by overexpression of genes that regulate oxidative phosphorylation, mitochondrial function and the electron transport chain, such as the nicotinamide adenine dinucleotide dehydrogenase (NADH) complex and cytochrome c/cytochrome c oxidase (COX) complex as well as adenosine triphosphate (ATP) synthase components. As it relates to the instant disclosure, the “OxPhos subtype” can also refer to DLBCLs that display upregulation in one or more genes selected from the group: SPCS3; SUCLG1; NDUFAB1; FADD; MRPS16; ATP6V1D; NDUFB1; NDUFB3; SEC 11A; and PARK7, many of which are associated with oxidative phosphorylation or the electron transport chain. Tumors of the OxPhos subtype also tend to have increased expression of proteosomal subunits and molecules regulating mitochondrial membrane potential and apoptosis. DLBCL of the OxPhos subtype tend to be sensitive to proteasome blockade or BCL2 family inhibition.

The “Host-receptor-type DLBCL” or “Host receptor-subtype” or “HR-subtype” refers to a subtype of DLBCL characterized by inflammatory/immune cell infiltratration, and includes a morphologically defined subset of T-cell/histiocyte-rich B-cell lymphomas (Monti, S. et al., 2005). As it relates to the instant disclosure, the “Host receptor-subtype” can also refer to DLBCLs that display upregulation in one or more genes selected from the group; PD-L1, CTLA4, IL15RA, GNS, PTPRM, AMICA, CFH, CD2, ITGAL, ACTN1, A2M and IL2RB, most of which are related to the adaptive T cell response of the immune system.

### Marker Genes

The present disclosure provides marker genes and sets of marker genes whose expression levels in a given sample are indicative of a particular subtype, e.g., B-cell receptor (BCR), Oxidative Phosphorylation (OxPhos) and Host Response (HR) of a DLBCL tumor. In some embodiments, the marker genes include those comprising the sequences of SEQ ID NOs: 1-141. Embodiments of the present disclosure encompass assaying expression of a plurality of marker genes selected from SEQ ID NOs: 1-141 for identification and diagnosis of DLBCL subtype. In some embodiments, a subset of marker genes selected from those comprising the sequences of SEQ ID NOs: 1-141 are assayed. In some embodiments, all 141 marker genes are assayed in methods and compositions disclosed herein.

Whilst the analysis of multiple genes is of use in developing a robust classification of tumors, each of the genes identified to be upregulated in a particular DLBCL subtype, and their encoded proteins can provide a target for the development of diagnostic and therapeutic agents. For example, marker genes relevant for diagnosis and as therapeutic targets for the BCR subtype include TRMU, CKAP5, PLCG2, FUS, WEE1, ITPR3, SNRPA, PKMYT1 and SUPT5H; for the OxPhos subtype include SPCS3, SUCLG1, NDUFAB1, FADD, MRPS16, ATP6V1D, NDUFB1, NDUFB3, SEC11A, and PARK7; for Host receptor-subtype include PD-L1, CTLA4, IL15RA, GNS, PTPRM, AMICA, CFH, CD2, ITGAL, ACTN1, A2M and IL2RB.

The methods described herein also encompass the detection of mutations within any of the marker genes or within a regulatory region of a marker gene. Such mutations may include, but are not limited to, deletions, additions, substitutions, and amplification of regions of genomic DNA that include all or part of a gene. Methods for detecting such mutations are well known in the art. Such mutations may result in overexpression or inappropriate expression of the gene.

The sequences of the marker genes of SEQ ID NOs: 1-141 and their gene products are known in the art and are publically available from databases such as GenBank. Table 1 shows the 141 marker genes and their leastic net weights. Table 2 below shows the marker genes and their accession numbers:

Table 1 shows final CCC signature derived from both Affymetrix discovery sets.

This list contains the final 141 genes that were used for Nanostring profiling both as Ensembl gene identifiers and gene symbols. In addition, the leastic net weights are shown for every gene in all three CCC classes.

| ensembl_gene_id | hgnc_symbol | BCR_weights | HR_weights | OxPhos_weights |
|---|---|---|---|---|
| ENSG00000000971 | CFH | −0.111669304 | 0.121280737 | 0 |
| ENSG00000004779 | NDUFAB1 | 0 | 0 | 0.113665942 |
| ENSG00000005075 | POLR2J | 0 | −0.002999596 | 0 |
| ENSG00000005844 | ITGAL | 0 | 0.078292993 | −0.071060048 |
| ENSG00000010810 | FYN | −0.01218821 | 0.111319597 | 0 |
| ENSG00000011376 | LARS2 | 0 | 0 | −0.172641936 |
| ENSG00000038427 | VCAN | −0.024913467 | 0 | 0 |
| ENSG00000054983 | GALC | −0.042882886 | 0 | 0 |
| ENSG00000065526 | SPEN | 0 | 0 | −0.106154451 |
| ENSG00000065911 | MTHFD2 | 0 | −0.05295845 | 0.06958494 |
| ENSG00000072110 | ACTN1 | 0 | 0.126041372 | −0.0563744 |
| ENSG00000072506 | HSD17B10 | 0 | −0.032430876 | 0 |
| ENSG00000072864 | NDE1 | 0 | 0 | −0.102042138 |
| ENSG00000077312 | SNRPA | 0.15183816 | 0 | −0.120713108 |
| ENSG00000078668 | VDAC3 | −0.015223578 | 0 | 0 |
| ENSG00000086102 | NFX1 | 0 | 0 | −0.061750331 |
| ENSG00000088827 | SIGLEC1 | 0 | 0.038325246 | −0.111099749 |
| ENSG00000089280 | FUS | 0.172031373 | 0 | −0.134990867 |
| ENSG00000095585 | BLNK | 0 | −0.007477651 | 0 |
| ENSG00000096433 | ITPR3 | 0.154330835 | 0 | −0.049598275 |
| ENSG00000099783 | HNRNPM | 0.074591338 | 0 | 0 |
| ENSG00000099795 | NDUFB7 | −0.040749836 | 0 | 0.074091435 |
| ENSG00000099995 | SF3A1 | 0.079491133 | 0 | −0.158174351 |
| ENSG00000100316 | RPL3 | 0 | −0.001236953 | 0 |
| ENSG00000100385 | IL2RB | 0 | 0.068681442 | 0 |
| ENSG00000100416 | TRMU | 0.455023374 | 0 | 0 |
| ENSG00000100554 | ATP6V1D | −0.059934286 | 0 | 0.10286005 |
| ENSG00000100600 | LGMN | 0 | 0.014794368 | 0 |
| ENSG00000102265 | TIMP1 | 0 | 0 | 0 |
| ENSG00000103653 | CSK | 0.090075999 | 0 | −0.044610332 |
| ENSG00000104852 | SNRNP70 | 0 | 0 | −0.114647333 |
| ENSG00000104897 | SF3A2 | 0.006574849 | 0 | −0.268770471 |
| ENSG00000105323 | HNRNPUL1 | 0.134759515 | 0 | −0.102446556 |
| ENSG00000105568 | PPP2R1A | 0.112462893 | 0 | −0.079832262 |
| ENSG00000105974 | CAV1 | −0.141496751 | 0.046560202 | 0 |
| ENSG00000106366 | SERPINE1 | 0 | 0 | 0 |
| ENSG00000108821 | COL1A1 | 0 | 0 | 0 |
| ENSG00000108883 | EFTUD2 | 0.101475197 | 0 | 0 |
| ENSG00000109390 | NDUFC1 | 0 | 0 | 0.050580664 |
| ENSG00000109861 | CTSC | −0.036510013 | 0 | 0 |
| ENSG00000111537 | IFNG | −0.026612392 | 0 | 0 |
| ENSG00000112695 | COX7A2 | −0.000356362 | 0 | 0.031337646 |
| ENSG00000115415 | STAT1 | −0.041667507 | 0.021977276 | 0 |
| ENSG00000116288 | PARK7 | 0 | 0 | 0.063175597 |
| ENSG00000116459 | ATP5F1 | 0 | 0 | 0.054287073 |
| ENSG00000116478 | HDAC1 | 0.047467887 | −0.035309508 | 0 |
| ENSG00000116824 | CD2 | −0.071914202 | 0.117579654 | 0 |
| ENSG00000119013 | NDUFB3 | 0 | 0 | 0.075905036 |
| ENSG00000120217 | CD274 | −0.268283572 | 0.252285546 | 0.015998026 |
| ENSG00000120742 | SERP1 | 0 | 0 | 0.073370491 |
| ENSG00000122406 | RPL5 | 0 | 0 | 0.052595774 |
| ENSG00000125356 | NDUFA1 | 0 | 0 | 0.047661835 |
| ENSG00000125730 | C3 | 0 | 0.046681274 | 0 |
| ENSG00000126267 | COX6B1 | 0 | −0.068309753 | 0.011406051 |
| ENSG00000127184 | COX7C | 0 | 0 | 0.013227086 |
| ENSG00000127540 | UQCR11 | 0 | 0 | 0.057225368 |
| ENSG00000127564 | PKMYT1 | 0.14664845 | −0.065537197 | 0 |
| ENSG00000129128 | SPCS3 | 0 | −0.001913276 | 0.150399779 |
| ENSG00000131368 | MRPS25 | 0.13839926 | 0 | 0 |
| ENSG00000131462 | TUBG1 | 0.039610581 | 0 | 0 |
| ENSG00000133226 | SRRM1 | 0 | 0 | −0.019537386 |
| ENSG00000134470 | IL15RA | 0 | 0.190618814 | 0 |
| ENSG00000134575 | ACP2 | 0 | 0.061195003 | 0 |
| ENSG00000135677 | GNS | 0 | 0.161063105 | 0 |
| ENSG00000135940 | COX5B | 0 | 0 | 0.020974836 |
| ENSG00000135972 | MRPS9 | 0 | −0.048675678 | 0 |
| ENSG00000136143 | SUCLA2 | 0 | 0 | 0.062129266 |
| ENSG00000136875 | PRPF4 | 0.119880689 | 0 | 0 |
| ENSG00000137462 | TLR2 | −0.025816734 | 0.01685016 | 0 |
| ENSG00000137822 | TUBGCP4 | 0.016629331 | 0 | 0 |
| ENSG00000138777 | PPA2 | 0 | 0 | 0.053252707 |
| ENSG00000139131 | YARS2 | 0 | −0.0006774 | 0.052184676 |
| ENSG00000140374 | ETFA | 0 | −0.023022078 | 0.050848788 |
| ENSG00000140612 | SEC11A | −0.048801756 | 0 | 0.066753191 |
| ENSG00000140740 | UQCRC2 | −0.009901236 | 0 | 0.046090443 |
| ENSG00000143933 | CALM2 | 0 | 0 | 0.026232814 |
| ENSG00000146282 | RARS2 | 0 | 0 | 0 |
| ENSG00000147669 | POLR2K | 0 | −0.033268916 | 0.05484841 |
| ENSG00000149131 | SERPING1 | 0 | 0.077673437 | −0.021593498 |
| ENSG00000149532 | CPSF7 | 0 | 0 | −0.066668669 |
| ENSG00000153563 | CD8A | 0 | 0.041908771 | 0 |
| ENSG00000154518 | ATP5G3 | 0 | 0 | 0 |
| ENSG00000155465 | SLC7A7 | 0 | 0.029377461 | 0 |
| ENSG00000156467 | UQCRB | 0 | 0 | 0.017355823 |
| ENSG00000156482 | RPL30 | 0 | 0 | 0.012448986 |
| ENSG00000157456 | CCNB2 | 0.051323627 | −0.100406962 | 0 |
| ENSG00000159189 | C1QC | 0 | 0.010656811 | 0 |
| ENSG00000159403 | C1R | −0.06575798 | 0.087464241 | 0 |
| ENSG00000160255 | ITGB2 | 0 | 0.064389901 | −0.021321062 |
| ENSG00000160299 | PCNT | 0.105309357 | 0 | −0.01621317 |
| ENSG00000160593 | AMICA1 | −0.103080516 | 0.145583997 | 0 |
| ENSG00000163541 | SUCLG1 | −0.042571176 | 0 | 0.143886686 |
| ENSG00000163599 | CTLA4 | 0 | 0.018336814 | 0 |
| ENSG00000164258 | NDUFS4 | 0 | 0 | 0.041149998 |
| ENSG00000164305 | CASP3 | 0 | −0.051534622 | 0.047185781 |
| ENSG00000164405 | UQCRQ | 0 | 0 | 0.031094793 |
| ENSG00000164733 | CTSB | −0.003707455 | 0.028028229 | 0 |
| ENSG00000165025 | SYK | 0.08745794 | 0 | 0 |
| ENSG00000165264 | NDUFB6 | 0 | 0 | 0.009657937 |
| ENSG00000165629 | ATP5C1 | 0 | 0 | 0.043216751 |
| ENSG00000166260 | COX11 | 0 | −0.005273297 | 0.044291541 |
| ENSG00000166340 | TPP1 | 0 | 0.033291302 | −0.158406899 |
| ENSG00000166483 | WEE1 | 0.158239941 | −0.138509701 | 0 |
| ENSG00000167283 | ATP5L | 0 | 0 | 0.020565273 |
| ENSG00000168040 | FADD | −0.033442264 | 0 | 0.110576975 |
| ENSG00000168827 | GFM1 | 0 | 0 | 0 |
| ENSG00000171860 | C3AR1 | −0.032486351 | 0.061787344 | 0 |
| ENSG00000173369 | C1QB | 0 | 0 | 0 |
| ENSG00000173372 | C1QA | −0.004032534 | 0 | 0 |
| ENSG00000173482 | PTPRM | 0 | 0.142608237 | 0 |
| ENSG00000173638 | SLC19A1 | 0.115962458 | −0.042405131 | 0 |
| ENSG00000174231 | PRPF8 | 0.002353493 | 0 | −0.080664401 |
| ENSG00000174748 | RPL15 | 0 | −0.045587813 | 0.026638774 |
| ENSG00000175110 | MRPS22 | 0 | 0 | 0.070005806 |
| ENSG00000175216 | CKAP5 | 0.211102398 | 0 | −0.013811342 |
| ENSG00000175899 | A2M | 0 | 0.105175089 | −0.055480413 |
| ENSG00000177733 | HNRNPA0 | 0.039539865 | 0 | −0.048577247 |
| ENSG00000182180 | MRPS16 | 0 | −0.149360988 | 0.109466588 |
| ENSG00000182199 | SHMT2 | 0.038271552 | −0.119909405 | 0 |
| ENSG00000182326 | C1S | −0.01446786 | 0 | 0 |
| ENSG00000182899 | RPL35A | 0 | −0.083275516 | 0.037531399 |
| ENSG00000183648 | NDUFB1 | 0 | 0 | 0.086766706 |
| ENSG00000184076 | UQCR10 | 0 | −0.057636451 | 0.014899169 |
| ENSG00000184752 | NDUFA12 | 0 | −0.015808966 | 0 |
| ENSG00000184983 | NDUFA6 | −0.018989287 | 0 | 0.069381273 |
| ENSG00000186340 | THBS2 | 0 | 0.02331951 | 0 |
| ENSG00000189043 | NDUFA4 | 0 | −0.006418169 | 0 |
| ENSG00000189091 | SF3B3 | 0.060042243 | 0 | −0.127303426 |
| ENSG00000196230 | TUBB | 0.09729406 | −0.032175027 | 0 |
| ENSG00000196235 | SUPT5H | 0.140918432 | 0 | −0.033855736 |
| ENSG00000197081 | IGF2R | 0 | 0.04306715 | −0.040745638 |
| ENSG00000197249 | SERPINA1 | 0 | 0.027594491 | 0 |
| ENSG00000197746 | PSAP | 0 | 0.102430994 | −0.003302258 |
| ENSG00000197766 | CFD | −0.008131938 | 0.044219833 | 0 |
| ENSG00000197943 | PLCG2 | 0.201397144 | −0.034473457 | 0 |
| ENSG00000198833 | UBE2J1 | 0 | −0.044057036 | 0.013882592 |
| ENSG00000204843 | DCTN1 | 0 | 0 | −0.035425147 |
| ENSG00000205937 | RNPS1 | 0 | 0 | −0.019993429 |
| ENSG00000213619 | NDUFS3 | 0 | 0 | 0.059918912 |
| ENSG00000256043 | CTSO | −0.083218015 | 0 | 0 |
| ENSG00000259494 | MRPL46 | 0 | 0 | 0.071449295 |

Table 2 Shows Final 141 Marker Genes and their Accession Numbers

| | | | GeneBank or RefSeq | |
|---|---|---|---|---|
| Gene Name | Gene ID# | UniProt ID # | (denoted by **NM_) | SEQ ID NO: |
| CFH | 3075 | P08603.4 | Y00716 | SEQ ID NO: 1 |
| NDUFAB1 | 4706 | O14561.3 | AF087660 | SEQ ID NO: 2 |
| POLR2J | 5439 | P52435.1 | X98433 | SEQ ID NO: 3 |
| ITGAL | 3683 | P20701.3 | **NM_001114380 | SEQ ID NO: 4 |
| FYN | 2534 | P06241.3 | AK056699 | SEQ ID NO: 5 |
| LARS2 | 23395 | Q15031.2 | AJ312685 | SEQ ID NO: 6 |
| VCAN | 1462 | P13611.3 | X15998 | SEQ ID NO: 7 |
| GALC | 2581 | P54803.3 | L23116 | SEQ ID NO: 8 |
| SPEN | 23013 | Q96T58.1 | **NM_015001.2 | SEQ ID NO: 9 |
| MTHFD2 | 10797 | P13995.2 | X16396 | SEQ ID NO: 10 |
| ACTN1 | 87 | P12814.2 | M95178 | SEQ ID NO: 11 |
| HSD17B10 | 3028 | Q99714.3 | U96132 | SEQ ID NO: 12 |
| NDE1 | 54820 | Q9NXR1.2 | AF124431 | SEQ ID NO: 13 |
| SNRPA | 6626 | P09012.3 | X06347 | SEQ ID NO: 14 |
| VDAC3 | 7419 | Q9Y277.1 | AF038962 | SEQ ID NO: 15 |
| NFX1 | 4799 | Q12986.2 | U19759 | SEQ ID NO: 16 |
| SIGLEC1 | 6614 | Q9BZZ2.2 | AF230073 | SEQ ID NO: 17 |
| FUS | 2521 | P35637.1 | AF071213 | SEQ ID NO: 18 |
| BLNK | 29760 | Q8WV28.2 | AF068180 | SEQ ID NO: 19 |
| ITPR3 | 3710 | Q14573.2 | D26351 | SEQ ID NO: 20 |
| HNRNPM | 4670 | P52272.3 | L03532 | SEQ ID NO: 21 |
| NDUFB7 | 4713 | P17568.4 | **NM_004146.5 | SEQ ID NO: 22 |
| SF3A1 | 10291 | Q15459.1 | X85237 | SEQ ID NO: 23 |
| RPL3 | 6122 | P39023.2 | AB007166 | SEQ ID NO: 24 |
| IL2RB | 3560 | P14784.1 | M26062 | SEQ ID NO: 25 |
| TRMU | 55687 | O75648 | AY062123 | SEQ ID NO: 26 |
| ATP6V1D | 51382 | Q9Y5K8.1 | AF145316 | SEQ ID NO: 27 |
| LGMN | 5641 | Q99538.1 | D55696 | SEQ ID NO: 28 |
| TIMP1 | 7076 | P01033.1 | **NM_003254.2 | SEQ ID NO: 29 |
| CSK | 1445 | P41240.1 | **NM_004383.2 | SEQ ID NO: 30 |
| SNRNP70 | 6625 | P08621.2 | **NM_003089.5 | SEQ ID NO: 31 |
| SF3A2 | 8175 | Q15428.2 | L21990 | SEQ ID NO: 32 |
| HNRNPUL1 | 11100 | Q9BUJ2.2 | AJ007509 | SEQ ID NO: 33 |
| PPP2R1A | 5518 | P30153.4 | **NM_014225.5 | SEQ ID NO: 34 |
| CAV1 | 857 | Q03135.4 | AF125348 | SEQ ID NO: 35 |
| SERPINE1 | 5054 | P05121.1 | M16006 | SEQ ID NO: 36 |
| COL1A1 | 1277 | P02452.5 | Z74615 | SEQ ID NO: 37 |
| NDUFC1 | 4717 | O43677.1 | AF047184 | SEQ ID NO: 38 |
| CTSC | 1075 | P53634.2 | AK223038 | SEQ ID NO: 39 |
| IFNG | 3458 | P01579.1 | **NM_000619.2 | SEQ ID NO: 40 |
| COX7A2 | 1347 | P14406.1 | X15822 | SEQ ID NO: 41 |
| STAT1 | 6772 | P42224.2 | **NM_007315.3 | SEQ ID NO: 42 |
| PARK7 | 11315 | Q99497.2 | D61380 | SEQ ID NO: 43 |
| ATP5F1 | 515 | P24539.2 | X60221 | SEQ ID NO: 44 |
| HDAC1 | 3065 | Q13547.1 | D50405 | SEQ ID NO: 45 |
| CD2 | 914 | P06729.2 | BC033583 | SEQ ID NO: 46 |
| NDUFB3 | 4709 | O43676.3 | AF047183 | SEQ ID NO: 47 |
| SERP1 | 27230 | Q9Y6X1.1 | AK125413 | SEQ ID NO: 48 |
| RPL5 | 6125 | P46777.3 | U14966 | SEQ ID NO: 49 |
| NDUFA1 | 4694 | O15239.1 | **NM_004541.3 | SEQ ID NO: 50 |
| C3 | 718 | P01024 | J04763 | SEQ ID NO: 51 |
| COX6B1 | 1340 | P14854.2 | BC001015 | SEQ ID NO: 52 |
| COX7C | 1350 | P15954.1 | BC001005 | SEQ ID NO: 53 |
| UQCR11 | 10975 | O14957.1 | D55636 | SEQ ID NO: 54 |
| PKMYT1 | 9088 | Q99640.1 | AK097642 | SEQ ID NO: 55 |
| SPCS3 | 60559 | P61009.1 | AK092634 | SEQ ID NO: 56 |
| TUBG1 | 7283 | P23258.2 | BC000619 | SEQ ID NO: 57 |
| SRRM1 | 10250 | Q8IYB3.2 | AF048977 | SEQ ID NO: 58 |
| IL15RA | 16169 | Q13261.1 | **NM_001271497.1 | SEQ ID NO: 59 |
| ACP2 | 53 | P11117.3 | X15525 | SEQ ID NO: 60 |
| GNS | 2799 | P15586.3 | **NM_002076.3 | SEQ ID NO: 61 |
| COX5B | 1329 | P10606.2 | BC006229 | SEQ ID NO: 62 |
| SUCLA2 | 8803 | Q9P2R7.3 | AF058953 | SEQ ID NO: 63 |
| PRPF4 | 9128 | O43172.2 | AF001687 | SEQ ID NO: 64 |
| TLR2 | 7097 | O60603.1 | U88878 | SEQ ID NO: 65 |
| TUBGCP4 | 27229 | Q9UGJ1.1 | AJ249677 | SEQ ID NO: 66 |
| PPA2 | 27068 | Q9H2U2.2 | **NM_176869.2 | SEQ ID NO: 67 |
| YARS2 | 51067 | Q9Y2Z4.2 | AF132939 | SEQ ID NO: 68 |
| ETFA | 2108 | P13804.1 | J04058 | SEQ ID NO: 69 |
| SEC11A | 23478 | P67812.1 | AF061737 | SEQ ID NO: 70 |
| UQCRC2 | 7385 | P22695.3 | J04973 | SEQ ID NO: 71 |
| CALM2 | 805 | P62158.2 | **NM_001743.5 | SEQ ID NO: 72 |
| POLR2K | 5440 | P53803.1 | **NM_005034.3 | SEQ ID NO: 73 |
| SERPING1 | 710 | P05155.2 | X54486 | SEQ ID NO: 74 |
| CPSF7 | 79869 | Q8N684.1 | **NM_024811.3 | SEQ ID NO: 75 |
| CD8A | 12525 | P01732.1 | **NM_001081110.2 | SEQ ID NO: 76 |
| ATP5G3 | 518 | P48201.1 | BC106881 | SEQ ID NO: 77 |
| SLC7A7 | 9056 | Q9UM01.2 | AF092032 | SEQ ID NO: 78 |
| UQCRB | 7381 | P14927.2 | X13585 | SEQ ID NO: 79 |
| RPL30 | 6156 | P62888.2 | **NM_000989.3 | SEQ ID NO: 80 |
| CCNB2 | 9133 | O95067.1 | AF002822 | SEQ ID NO: 81 |
| C1R | 715 | P00736.2 | M14058 | SEQ ID NO: 82 |
| ITGB2 | 3689 | P05107.2 | AK222505 | SEQ ID NO: 83 |
| PCNT | 5116 | O95613.4 | AB007862 | SEQ ID NO: 84 |
| SUCLG1 | 8802 | P53597.4 | Z68204 | SEQ ID NO: 85 |
| CTLA4 | 1493 | P16410.3 | **NM_005214.4 | SEQ ID NO: 86 |
| NDUFS4 | 4724 | O43181.1 | AF020351 | SEQ ID NO: 87 |
| CASP3 | 836 | P42574.2 | BCO16926 | SEQ ID NO: 88 |
| UQCRQ | 27089 | O14949.4 | BC001390 | SEQ ID NO: 89 |
| CTSB | 1508 | P07858.3 | M14221 | SEQ ID NO: 90 |
| SYK | 6850 | P43405.1 | L28824 | SEQ ID NO: 91 |
| NDUFB6 | 4712 | O95139.3 | AF035840 | SEQ ID NO: 92 |
| ATP5C1 | 509 | P36542.1 | D16561 | SEQ ID NO: 93 |
| COX11 | 1353 | Q9Y6N1.3 | AF044321 | SEQ ID NO: 94 |
| TPP1 | 1200 | O14773.2 | AF017456 | SEQ ID NO: 95 |
| WEE1 | 7465 | P30291.2 | X62048 | SEQ ID NO: 96 |
| ATP5L | 10632 | O75964.3 | AF092124 | SEQ ID NO: 97 |
| FADD | 8772 | Q13158.1 | U24231 | SEQ ID NO: 98 |
| GFM1 | 85476 | Q96RP9.2 | AF309777 | SEQ ID NO: 99 |
| C3AR1 | 12267 | Q16581.2 | **NM_009779.2 | SEQ ID NO: 100 |
| C1QB | 713 | P02746.3 | X03084 | SEQ ID NO: 101 |
| C1QA | 12259 | P02745.2 | **NM_007572.2 | SEQ ID NO: 102 |
| PTPRM | 5797 | P28827.2 | X58288 | SEQ ID NO: 103 |
| SLC19A1 | 6573 | P41440.3 | U15939 | SEQ ID NO: 104 |
| PRPF8 | 10594 | Q6P2Q9.2 | AB007510 | SEQ ID NO: 105 |
| RPL15 | 6138 | P61313.2 | AB007173 | SEQ ID NO: 106 |
| MRPS22 | 56945 | P82650.1 | AF226045 | SEQ ID NO: 107 |
| CKAP5 | 9793 | Q14008.3 | **NM_014756.3 | SEQ ID NO: 108 |
| A2M | 2 | P01023.3 | BX647329 | SEQ ID NO: 109 |
| HNRNPA0 | 10949 | Q13151.1 | U23803 | SEQ ID NO: 110 |
| MRPS16 | 51021 | Q9Y3D3.1 | AB051351 | SEQ ID NO: 111 |
| SHMT2 | 6472 | P34897.3 | AK223555 | SEQ ID NO: 112 |
| C1S | 716 | P09871.1 | **NM_001734.4 | SEQ ID NO: 113 |
| RPL35A | 6165 | P18077.2 | X52966 | SEQ ID NO: 114 |
| NDUFB1 | 4707 | O75438.1 | BC104672 | SEQ ID NO: 115 |
| UQCR10 | 29796 | Q9UDW1.3 | AB028598 | SEQ ID NO: 116 |
| NDUFA6 | 4700 | P56556.3 | AF047182 | SEQ ID NO: 117 |
| THBS2 | 21826 | P35442.2 | **NM_011581.3 | SEQ ID NO: 118 |
| NDUFA4 | 4697 | O00483.1 | U94586 | SEQ ID NO: 119 |
| SF3B3 | 23450 | Q15393.4 | AJ001443 | SEQ ID NO: 120 |
| TUBB | 203068 | P07437.2 | AB062393 | SEQ ID NO: 121 |
| SUPT5H | 6829 | O00267.1 | U56402 | SEQ ID NO: 122 |
| IGF2R | 3482 | P11717.3 | J03528 | SEQ ID NO: 123 |
| SERPINA1 | 5265 | P01009.3 | X01683 | SEQ ID NO: 124 |
| PSAP | 5660 | P07602.2 | BC004275 | SEQ ID NO: 125 |
| CFD | 1675 | P00746.5 | M84526 | SEQ ID NO: 126 |
| PLCG2 | 5336 | P16885.4 | **NM_002661.4 | SEQ ID NO: 127 |
| UBE2J1 | 51465 | Q9Y385.2 | AJ245898 | SEQ ID NO: 128 |
| DCTN1 | 1639 | Q14203.3 | **NM_004082.4 | SEQ ID NO: 129 |
| RNPS1 | 10921 | Q15287.1 | AF015608 | SEQ ID NO: 130 |
| NDUFS3 | 4722 | O75489.1 | AF067139 | SEQ ID NO: 131 |
| CTSO | 1519 | P43234.1 | X77383 | SEQ ID NO: 132 |
| MRPL46 | 26589 | Q9H2W6.1 | AF210056 | SEQ ID NO: 133 |
| EFTUD2 | 9343 | Q15029.1 | D21163 | SEQ ID NO: 134 |
| CD274 | 29126 | Q9NZQ7.1 | AF177937 | SEQ ID NO: 135 |
| MRPS25 | 64432 | P82663.1 | AB061208 | SEQ ID NO: 136 |
| MRPS9 | 64965 | P82933.2 | **NM_182640.2 | SEQ ID NO: 137 |
| RARS2 | 57038 | Q5T160.1 | AK093934 | SEQ ID NO: 138 |
| C1QC | 714 | P02747.3 | AK057792 | SEQ ID NO: 139 |
| AMICA1 | 270152 | Q86YT9.1 | **NM_001005421.4 | SEQ ID NO: 140 |
| NDUFA12 | 55967 | Q9UI09.1 | BC005936 | SEQ ID NO: 141 |

### Methods of Assaying Expression Levels of Genes

The expression level of a marker gene can be measured in a number of ways, including, but not limited to measuring the mRNA encoded by the selected genes, measuring the amount of protein encoded by the selected genes, and/or measuring the activity of the protein encoded by the selected genes.

### Measuring a Level of Nucleic Acid Gene Product

The mRNA level for a marker gene can be determined in in situ and in in vitro formats using methods known in the art. Many of such methods use isolated RNA. For in vitro methods, any RNA isolation technique that does not select against the isolation of mRNA can be utilized for the purification of RNA from the cancer cells (see, e.g., Ausubel et al., eds., 1987-1997, Current Protocols in Molecular Biology, John Wiley & Sons, Inc., New York). Additionally, large numbers of tissue samples can readily be processed using techniques well known to those of skill in the art, such as, for example, the single-step RNA isolation process of Chomczynski (1989, U.S. Pat. No. 4,843,155). The isolated RNA can be used in hybridization or nucleic acid amplification assays that include, but are not limited to, Southern or Northern analyses, microarray hybridization, and polymerase chain reaction analyses, e.g., reverse transcription PCR (RT-PCR) assays.

The first step for gene expression analysis is the isolation of RNA or mRNA from a target sample. The starting material is typically total RNA isolated from human tumors, and corresponding normal tissue(s) or cell lines, respectively. If the source of RNA is a primary tumor, RNA can be extracted, for example, from frozen or archived paraffin-embedded and fixed (e.g., formalin-fixed) tissue samples. General methods for RNA extraction are well known in the art and are described in standard textbooks of molecular biology, including Ausubel et al., Current Protocols of Molecular Biology (Wiley and Sons, 1997). Methods for RNA extraction from paraffin embedded tissues are disclosed, for example, by M. Cronin, Am J. Pathol 164(1):35-42 (2004), the contents of which are incorporated herein. In particular, RNA isolation can be performed using kits and reagents from commercial manufacturers according to the manufacturer's instructions. For example, total RNA from cells in culture can be isolated using RNeasy® mini-columns (Qiagen GmbH Corp.). Other commercially available RNA isolation kits include MasterPure™ Complete DNA and RNA Purification Kit (EPICENTRE® Biotechnologies, Madison, Wis.), mirVana (Applied Biosystems, Inc.), and Paraffin Block RNA Isolation Kit (Ambion, Inc.). Total RNA from tissue samples can be isolated using RNA STAT-60™ (IsoTex Diagnostics, Inc., Friendswood Tex.). RNA prepared from tumor tissue can be isolated, for example, by cesium chloride density gradient centrifugation. (RNeasy is a registered trademark of Qiagen). Methods of isolating RNA for expression analysis from blood, plasma and serum (See for example, Tsui N B et al. (2002) 48, 1647-53) and from urine (See for example, Boom R et al. (1990) J Clin Microbiol. 28, 495-503 and reference cited therein) have been described. Methods of isolating RNA from paraffin-embedded tissue, as well as mRNA isolation, purification, primer extension and amplification are also provided in various published journal articles. (See, e.g., T. E. Godfrey et al., J. Molec. Diagnostics 2: 84-91 (2000); K. Specht et al., Am. J. Pathol. 158: 419-29 (2001), M. Cronin, et al., Am J Pathol 164:35-42 (2004)). For in situ hybridization methods, RNA does not need to be isolated from the cancer cells prior to detection. In such methods, a cell or tissue sample is prepared and processed using known histological methods.

### Expression Analysis Based on Hybridization

One preferred method for the determination of mRNA levels involves contacting the isolated RNA with a nucleic acid molecule (probe) that can hybridize to the RNA encoded by the gene being detected. In one format, the RNA is immobilized on a solid surface and contacted with the probes, for example by running the isolated RNA on an agarose gel and transferring the RNA from the gel to a membrane, such as nitrocellulose. In an alternative format, the probes are immobilized on a solid surface and the RNA sample is contacted with the probes, for example in an array platform such as a microarray (e.g., Affymetrix gene array). A skilled artisan can readily adapt known RNA detection methods for use in detecting the level of mRNA encoded by marker genes described herein.

In brief, a DNA microarray, also referred to as a DNA chip, is a microscopic array of DNA fragments, such as synthetic oligonucleotides, disposed in a defined pattern on a solid support, and amenable to analysis by standard hybridization methods (see Schena, BioEssays 18:427 (1996)). Exemplary microarrays and methods for their manufacture and use are set forth in T. R. Hughes et al., Nature Biotechnology 9:342-347 (2001). A number of different microarray configurations and methods for their production are known to those of skill in the art and are disclosed in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 6,022,963; 6,077,674; and U.S. Pat. No. 6,156,501; Shena, et al, Tibtech 6:301-306, 1998; Duggan, et al., Nat. Genet. 2:10-14, 1999; Bowtell, et al., Nat. Genet. 21:25-32, 1999; Lipshutz, et al., Nat. Genet. 21:20-24, 1999; Blanchard, et al, Biosensors and Bioelectronics 77:687-90, 1996; Maskos, et al., Nucleic Acids Res. 2:4663-69, 1993; and Hughes, et al., Nat. Biotechnol. 79:342-347, 2001. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219; the disclosures of which are herein incorporated by reference.

In one embodiment, an array of oligonucleotide probes can be arranged on a solid support. Exemplary solid supports include glass, plastics, polymers, metals, metalloids, ceramics, organics, etc. Using chip masking technologies and photoprotective chemistry, it is possible to generate ordered arrays of nucleic acid probes. These arrays, which are known, for example, as very large scale immobilized polymer arrays (“VLSIPS®” arrays), can include millions of defined probe regions on a substrate having an area of about 1 cm2 to several cm2, thereby incorporating from a few to millions of probes (see, e.g., U.S. Pat. No. 5,631,734).

Labeled nucleic acids can be contacted with the array under conditions sufficient for binding between the target nucleic acid and the probe on the array. In one embodiment, the hybridization conditions can be selected to provide for the desired level of hybridization specificity; that is, conditions sufficient for hybridization to occur between the labeled nucleic acids and probes on the microarray.

Hybridization can be carried out in conditions permitting substantially specific hybridization. The length and GC content of the nucleic acid will determine the thermal melting point and thus, the hybridization conditions necessary for obtaining specific hybridization of the probe to the target nucleic acid. These factors are well known to a person of skill in the art, and may also be tested in assays. An extensive guide to nucleic acid hybridization may be found in Tijssen, et al. (Laboratory Techniques in Biochemistry and Molecular Biology, Vol. 24: Hybridization With Nucleic Acid Probes, P. Tijssen, ed.; Elsevier, N.Y. (1993)). The methods described above will result in the production of hybridization patterns of labeled target nucleic acids on the array surface. The resultant hybridization patterns of labeled nucleic acids can be visualized or detected in a variety of ways, with the particular manner of detection selected based on the particular label employed. Representative detection options include scintillation counting, autoradiography, fluorescence measurement, light emission measurement, light scattering, and the like.

One such method of detection utilizes an array scanner that is commercially available (Affymetrix, Santa Clara, Calif.), for example, the 417® Arrayer, the 418® Array Scanner, or the Agilent Gene Array® Scanner. This scanner is controlled from a system computer with an interface and easy-to-use software tools. The output can be directly imported into or directly read by a variety of software applications. Exemplary scanning devices are described in, for example, U.S. Pat. Nos. 5,143,854 and 5,424,186. In yet other embodiments, the assay format employs direct mRNA capture with branched DNA (QuantiGene™, Panomics) or Hybrid Capture™ (Digene).

In some embodiments the method to measure transcript abundance for the marker genes listed in Table 1 utilizes the nCounter® Analysis System marketed by NanoString® Technologies (Seattle, Wash. USA). This system is described by Geiss et al., Nature Biotechnol. 2(3):317-325 (2008), and U.S. Pat. No. 7,919,237. Typically the system utilizes a pair of probes, including a capture probe and a reporter probe, each comprising a 35- to 50-base sequence complementary to the transcript to be detected. The capture probe additionally includes a short common sequence coupled to an immobilization tag, e.g. an affinity tag that allows the complex to be immobilized for data collection. The reporter probe additionally includes a detectable signal or label, e.g., to a color-coded tag. Following hybridization, excess probes are removed from the sample, and hybridized probe/target complexes are aligned and immobilized via the affinity or other tag in a cartridge. The samples are then analyzed, for example using a digital analyzer or other processor adapted for this purpose. Generally, the color-coded tag on each transcript is counted and tabulated for each target transcript to yield the expression level of each transcript in the sample. This system allows measuring the expression of hundreds of unique gene transcripts in a single multiplex assay using capture and reporter probes designed by NanoString.

Another example of an array technology suitable for use in measuring expression of the marker genes described herein is the ArrayPlate™ assay technology sold by HTG Molecular, Tucson Ariz., and described in Martel, R. R., et al, Assay and Drug Development Technologies 1(1):61-71, 2002. In brief, this technology combines a nuclease protection assay with array detection. Cells in microplate wells are subjected to a nuclease protection assay. Cells are lysed in the presence of probes that bind targeted mRNA species. Upon addition of SI nuclease, excess probes and unhybridized mRNA are degraded, so that only mRNA:probe duplexes remain. Alkaline hydrolysis destroys the mRNA component of the duplexes, leaving probes intact. After the addition of a neutralization solution, the contents of the processed cell culture plate are transferred to another plate termed a programmed ArrayPlate™. ArrayPlates™ contain a 16-element array at the bottom of each well. Each array element comprises a position-specific anchor oligonucleotide that remains the same from one assay to the next. The binding specificity of each of the 16 anchors is modified with a programming linker oligonucleotide, which is complementary at one end to an anchor and at the other end to a nuclease protection probe. During a hybridization reaction, probes transferred from the culture plate are captured by immobilized programming linker. Captured probes are labeled by hybridization with a detection linker oligonucleotide, which is in turn labeled with a detection conjugate that incorporates peroxidase. The enzyme is supplied with a chemiluminescent substrate, and the enzyme-produced light is captured in a digital image. Light intensity at an array element is a measure of the amount of corresponding target mRNA present in the original cells.

### Expression Measurement Methods Based on Target Amplification

An alternative method for determining the level of mRNA in a sample that is encoded by one of the marker genes disclosed herein involves the process of nucleic acid amplification, e.g., by PCR. RT-PCR, RTQ-PCR, spPCR and qPCR (the experimental embodiment set forth in Mullis, 1987, U.S. Pat. No. 4,683,202), ligase chain reaction (Barany, 1991, Proc. Natl. Acad. Sci. USA 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), or any other nucleic acid amplification method, followed by the detection of the amplified molecules using techniques well known to those of skill in the art (including, but not limited to microarray hybridization as discussed herein above). These detection schemes are especially useful for the detection of nucleic acid molecules if such molecules are present in very low numbers.

One detection method involves performing quantitative PCR on a sample using a set of oligonucleotide primers designed to amplify the genes of SEQ ID NOs 1-141 or subset thereof (Considerations for primer design are well known in the art and are described, for example, in Newton, et al. (eds.) PCR: Essential data Series, John Wiley & Sons; PCR Primer: A Laboratory Manual, Cold Spring Harbor Laboratory Press, Cold Spring Harbor, N.Y., 1995; White, et al. (eds.) PCR Protocols: Current methods and Applications, Methods in Molecular Biology, The Humana Press, Totowa, N.J., 1993. In addition, a variety of computer programs known in the art may be used to select appropriate primers.). A target mRNA can be amplified by reverse transcribing the mRNA into cDNA, and then performing PCR (reverse transcription-PCR or RT-PCR). Alternatively, a single enzyme may be used for both steps as described in U.S. Pat. No. 5,322,770. The fluorogenic 5′ nuclease assay, known as the TaqMan® assay (Roche Molecular Systems, Inc.), is a powerful and versatile PCR-based detection system for nucleic acid targets. For a detailed description of the TaqMan assay, reagents and conditions for use therein, see, e.g., Holland et al., Proc. Natl. Acad. Sci., U.S.A. (1991) 88:7276-7280; U.S. Pat. Nos. 5,538,848, 5,723,591, and 5,876,930, all incorporated herein by reference in their entireties.

In some embodiments, assaying a tumor sample for expression of the genes in Table 1, or gene signatures derived therefrom, employs detection and quantification of RNA levels in real-time using nucleic acid sequence based amplification (NASBA) combined with molecular beacon detection molecules. NASBA is described, e.g., in Compton J., Nature 350 (6313):91-92 (1991). NASBA is a single-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in step 1, such that the reaction is cyclic.

According to one embodiment of the methods described herein the sample is distributed into multiple vessels, e.g., multiple wells of a 396 well microtiter plate. For example, a pair of primers designed to amplify a portion of a gene in one of the inventive gene sets or subsets is added to each well, and PCR amplification is performed. The resulting product can then be detected using any of a number of methods known in the art depending upon the particular method of performing quantitative PCR that is employed. A 396 well plate permits at least duplicates of each of SEQ ID NO: 1-141 if so desired, to be run in a single assay. Primers sufficient for amplification of genes that allow quantitation of different cell types within the sample may also be included in the set of primers.

### Measuring Levels of a Polypeptide Gene Product

As an alternative to nucleic acid detection, it is contemplated that expression of the markers described herein can be detected at the protein level. A variety of methods can be used to determine the levels of proteins encoded by the selected signature genes. In general, these methods include contacting an agent that selectively binds to the protein, such as an antibody, with a sample, and evaluating the level of target protein in the sample. In a preferred embodiment, the antibody or an appropriate secondary antibody bears a detectable label. Antibodies can be polyclonal, or more preferably, monoclonal. An intact antibody, or a fragment thereof (e.g., Fab or F(ab′)2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are known in the art, as are methods of quantifying levels of proteins detected thereby.

The detection methods can be used to detect proteins encoded by one or more marker genes disclosed herein in a biological sample in vitro as well as in vivo. Exemplary in vitro techniques for detection of protein include enzyme linked immunosorbent assays (ELISAs), immunoprecipitations, immunofluorescence, enzyme immunoassay (EIA), radioimmunoassay (RIA), targeted mass-spectrometry, immunolabelling and Western blot analysis. In vivo techniques for detection of protein include introducing into a subject a labeled antibody directed to proteins encoded by one or more marker genes disclosed herein. For example, the antibody can be labeled with a radioactive marker whose presence and location in a subject can be detected by standard imaging techniques. In general, assays in which the antibodies may be employed include methods that use the antibody to detect the polypeptide in a tissue sample (e.g., a tumor sample), cell sample, body fluid sample (e.g., serum), cell extract, etc. Such methods typically involve the use of a labeled secondary antibody that recognizes the primary antibody (i.e., the antibody that binds to the polypeptide being detected). Depending upon the nature of the sample, appropriate methods include, but are not limited to, immunohistochemistry, radioimmunoassay, ELISA, immunoblotting, and FACS analysis. In the case where the polypeptide is to be detected in a tissue sample, e.g., a biopsy sample, immunohistochemistry is a particularly appropriate detection method. However, methods that pass a protein extract from a biopsy sample over an array of immobilized probes (e.g., antibodies) will permit simultaneous detection of a number of proteins in the sample. Techniques for obtaining tissue and cell samples and performing immunohistochemistry and FACS are well known in the art. In general, such assays will include a negative control, which can involve applying the test to normal tissue so that the signal obtained thereby can be compared with the signal obtained from the sample being assayed. In assays in which a secondary antibody is used to detect the antibody that binds to the polypeptide of interest, an appropriate negative control can involve performing the test on a portion of the sample with the omission of the antibody that binds to the polypeptide to be detected, i.e., with the omission of the primary antibody. Antibodies suitable for use as diagnostics generally exhibit high specificity for the target polypeptide and low background. In general, monoclonal antibodies are preferred for diagnostic purposes.

Accordingly, in some embodiments of the methods and compositions described herein, detection or determination of protein expression product level comprises the use of antibody or aptamer probes that selectively bind to the protein to be detected. In some embodiments, antibody micro-arrays can comprise antibodies and/or aptamers that selectively bind to a protein encoded by one or more genes of SEQ ID NO: 1-141 or subset thereof. The methods described herein encompass the use of protein arrays, including antibody arrays, for detection of the polypeptide. The use of antibody arrays is described, for example, in Haab, B., et al., “Protein microarrays for highly parallel detection and quantitation of specific proteins and antibodies in complex solutions”, Genome Biol. 2001; 2(2), 2001. Other types of protein arrays are known in the art. In addition, in certain embodiments, the polypeptides are detected using other modalities known in the art for the detection of polypeptides, such as aptamers (Aptamers, Molecular Diagnosis, Vol. 4, No. 4, 1999), reagents derived from combinatorial libraries for specific detection of proteins in complex mixtures, random peptide affinity reagents, etc. In general, any appropriate method for detecting a polypeptide can be used in conjunction with the methods described herein, although antibodies may represent a particularly appropriate modality. Antibodies (either monoclonal or polyclonal) can be generated by methods well known in the art and described, for example, in Harlow, E., Lane, E., and Harlow, E., (eds.) Using Antibodies: A Laboratory Manual, Cold Spring Harbor Laboratory Press, Cold Spring Harbor, 1998. Details and references for the production of antibodies based on an inventive polypeptide can also be found in U.S. Pat. No. 6,008,337. Antibodies can include, but are not limited to, polyclonal, monoclonal, chimeric (e.g., “humanized”), and single chain antibodies, and Fab fragments, antibodies generated using phage display technology, etc.

Samples

In some embodiments of the methods and compositions described herein, DLBCL subtype is assessed through the evaluation of expression levels of genes of SEQ ID NOs 1-141 or a subset thereof, in a sample from a subject. A subject can be a patient, a study participant, a control subject, a screening subject, or any other class of individual from whom a sample is obtained and assessed in the context of the disclosure. Accordingly, a subject can be diagnosed with DLBCL cancer, can present with one or more symptoms thereof or a predisposing factor, such as a family (genetic) or medical history (medical) factor, for DLBCL, or can be undergoing treatment or therapy for DLBCL. Alternatively, a subject can be healthy with respect to any of the aforementioned factors or criteria. It will be appreciated that the term “healthy” as used herein, is relative to DLBCL status, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more cancers other than DLBCL. However, the healthy controls are preferably free of any cancer.

In some embodiments, the methods for diagnosing DLBCL subtypes include assaying a sample comprising a cancer cell or tissue. By “sample” is intended any sampling of cells, tissues, or bodily fluids in which expression of selected genes can be detected. Examples of such samples include, but are not limited to, biopsies and smears. Most often, the sample will include a tumor biopsy sample. However, other sample types are contemplated. Bodily fluids potentially useful in the methods and compositions disclosed herein include blood, lymph, urine, saliva, nipple aspirates, gynecological fluids, or any other bodily secretion or derivative thereof. Blood can include whole blood, plasma, serum, or any derivative of blood. In some embodiments, the sample includes bone marrow or lymph node biopsy. In some embodiments, the sample comprises total nucleic acids and/or proteins isolated from the samples in which expression of selected genes can be detected. Samples can be obtained from a subject by a variety of techniques including, for example, by scraping or swabbing an area, by using a needle to aspirate cells or bodily fluids, or by removing a tissue sample (i.e., biopsy). Methods for collecting various biological samples are well known in the art. In some embodiments, a sample is obtained by, for example, fine needle aspiration biopsy, core needle biopsy, or excisional biopsy. Fixative and staining solutions can be applied to the cells or tissues for preserving the specimen and for facilitating examination. Biological samples can be transferred to a glass slide for viewing under magnification. In one embodiment, the biological sample is a formalin-fixed, paraffin-embedded tumor tissue sample. In various embodiments, the tissue sample is obtained from a pathologist guided tissue core sample.

Compositions

Provided herein are compositions comprising an array comprising probes for use in diagnosis of DLBCL-subtype. Exemplary arrays include but are not limited to cDNA arrays, oligonucleotide arrays, and protein arrays. cDNA microarrays consist of multiple (often thousands) different cDNA probes spotted (often using a robotic spotting device) onto known locations on a solid support, such as a glass microscope slide. The cDNAs are typically obtained by PCR amplification of plasmid library inserts using primers complementary to the vector backbone portion of the plasmid or to the gene itself for genes where sequence is known. PCR products suitable for production of microarrays are typically between 0.5 and 2.5 kB in length. Full length cDNAs, or partial cDNA, including sequence unique to the gene of interest can be chosen. As will be appreciated by one of ordinary skill in the art, in general the cDNAs contain sufficient sequence information to uniquely identify a gene within the human genome. Furthermore, in general the cDNAs are of sufficient length to hybridize, preferably specifically and yet more preferably uniquely, to cDNA obtained from mRNA derived from a single gene under the hybridization conditions of the experiment or assay.

Isolated oligonucleotides for use in oligonucleotide arrays are preferably from about 15 to about 150 nucleotides, more preferably from about 20 to about 100 nucleotides in length. The oligonucleotide can be a naturally occurring oligonucleotide or a synthetic oligonucleotide. Oligonucleotides can be prepared by the phosphoramidite method (Beaucage and Carruthers, Tetrahedron Lett. 22:1859-62, 1981), or by the triester method (Matteucci, et al., J. Am. Chem. Soc. 103:3185, 1981), or by other chemical methods known in the art.

Additional information describing methods for fabricating and using microarrays is found in U.S. Pat. No. 5,807,522, which is herein incorporated by reference. Instructions for constructing microarray hardware (e.g., arrayers and scanners) using commercially available parts can be found at cmgm.stanford.edu/pbrown/ and in Cheung, V., Morley, M., Aguilar, F., Massimi, A., Kucherlapati, R., and Childs, G., Making and reading microarrays, Nature Genetics Supplement, 21:15-19, 1999, which are herein incorporated by reference. Additional discussions of microarray technology and protocols for preparing samples and performing microarray experiments are found in, for example, “DNA arrays for analysis of gene expression”, Methods Enzymol, 303:179-205, 1999; “Fluorescence-based expression monitoring using microarrays”, Methods Enzymol, 306: 3-18, 1999; and M. Schena (ed.), “DNA Microarrays: A Practical Approach”, Oxford University Press, Oxford, U K, 1999. Descriptions of how to use an arrayer and the associated software are found at cmgm.stanford.edu/pbrown/mguide/arrayerHTML/ArrayerDocs.html, which is herein incorporated by reference.

Any hybridization assay format can be used, including solution-based and solid support-based assay formats. Solid supports containing oligonucleotide probes for genes of interest can be filters, polyvinyl chloride dishes, silicon or glass based chips, etc. Such supports and hybridization methods are widely available, for example, those disclosed by Beattie (WO 95/11755). Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There can be, for example, about 2, 10, 100, 1000 to 10,000; 100,000 or 400,000 of such features on a single solid support. The solid support, or the area within which the probes are attached may be on the order of a square centimeter.

Methods of forming high density arrays of oligonucleotides with a minimal number of synthetic steps are known. An oligonucleotide array can be synthesized on a solid substrate by a variety of methods, including, but not limited to, light-directed chemical coupling, and mechanically directed coupling. See, Pirrung et al., (1992) U.S. Pat. No. 5,143,854; Fodor et al., (1998) U.S. Pat. No. 5,800,992; Chee et al, (1998) U.S. Pat. No. 5,837,832 and Fodor et al. (WO 93/09668). Oligonucleotide probe arrays for expression monitoring can be made and used according to any techniques known in the art (see for example, Lockhart et al., (1996) Nat. Biotechnol. 14, 1675-1680; McGall et al., (1996) Proc. Nat. Acad. Sci. USA 93, 13555-13460). Such probe arrays can contain at least one or more oligonucleotides that are complementary to or hybridize to one or more of the genes described herein. Such arrays can also contain oligonucleotides that are complementary or hybridize to at least about 4, 5, 6, 7, 8, 9, 10, 15, 20, 30, 50, 70, 90, 110, 120, 130, or all 141 of the genes described herein. Control nucleic acids can also be spotted on the array. Quantitative nuclease protection arrays (qNPA), such as those described in U.S. Pat. Nos. 6,232,066 and 6,238,869 can be employed, wherein probes comprise one or more of genes disclosed in Tables 1-2 or complements thereof. Methods for conducting qNPA assays in fixed tissue samples are described in PCT/US08/58837, which is incorporated herein by reference in its entirety.

As discussed above nucleic acid amplification can be used for detection of target/marker genes in a sample. Accordingly, in some embodiments of the compositions and kits herein, the probes can be nucleic acid primers. The design of appropriate oligonucleotide primer pairs is well within the level of skill in the art, based on the specification and the recited sequence information provided for the relevant genes. In some embodiments, the probes comprise a target specific sequence that hybridize to no more than one gene under stringent hybridization conditions (so called “high stringency conditions”). In some embodiments, each probe in the probe pair comprises a target specific sequence that hybridizes to no more than one gene under stringent hybridization conditions, and wherein the target-specific sequences in each pair hybridizes to different regions of the same gene. For example, as is well known in the art, oligonucleotide primers can be used in various assays (PCR, RT-PCR, RTQ-PCR, spPCR, qPCR, and allele-specific PCR, etc.) to amplify portions of a target to which the primers are complementary. Thus, a primer pair would include both a “forward” and a “reverse” primer, one complementary to the sense strand and one complementary to an “antisense” strand, and designed to hybridize to the target so as to be capable of generating a detectable amplification product from the target of interest (e.g., gene product of one or more genes corresponding to SEQ ID NO: 1-141) when subjected to amplification conditions. The sequences of each of the target nucleic acids are provided (see accession numbers) herein, and thus, based on the teachings of the present specification, those of skill in the art can design appropriate primer pairs complementary to the target of interest (or complements thereof). In various embodiments, each member of the primer pair is a single stranded DNA polynucleotide at least 15, 16, 17, 18, 19, 20, 21, 22, 23, 24, 25, 26, 27, 28, 29, 30, 31, 35, 40, 45, 50, or more nucleotides in length that are fully complementary to the expression product target. In all embodiments, the nucleic acid primers are optionally detectably labeled using standard methods in the art. PCR, RT-PCR, and other amplification techniques, including quantitative amplification techniques, can be carried out using methods well known to those of skill in the art based on the teachings herein.

As also discussed herein above, methods that measure protein levels can also be used to evaluate target gene/marker expression. Accordingly, in one aspect, the instant disclosure provides antibody arrays or micro-arrays comprising or consisting of one or more antibodies and/or aptamers (nucleic acids or peptides that bind a specific target molecule) that selectively bind to a protein encoded by the gene(s) of any one of SEQ ID NOs 1-141, arrayed on a solid support. Preferably, such arrays can comprise a plurality of antibodies and/or aptamers which selectively bind to at least 2, 5, 15, 10, 20, 30, 40, 50, 60, 70, 80, 90, 100, 110, 120, 130, 140, 141, of the protein expression products encoded by genes of SEQ ID Nos.: 1-141. In one preferred embodiment, the antibody and/or aptamer arrays contain probes for a subset of gene of SEQ ID Nos 1-141. Embodiments disclosed herein for other aspects apply to this aspect as well unless the context clearly dictates otherwise, and may be combined with embodiments described for this aspect. For example, the antibody and/or aptamer arrays preferably comprise or consist of probes for the various subsets of genes for use described in various aspects disclosed herein. Each of these embodiments is useful in methods for diagnosing DLBCL subtype and DLBCL treatment based upon the subtype determined. In various further embodiments, the antibody and/or aptamer arrays may further comprise probes for other genes listed in Table 4. Antibody and/or aptamer molecules can comprise detectable labels; methods for labeling such molecules are known in the art.

All arrays of the present disclosure can be formed on any suitable solid surface material. Examples of such solid surface materials include, but are not limited to, beads, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose, polyacrylamides, methylmethracrylate polymers; sol gels; porous polymer hydrogels; nanostructured surfaces; nanotubes (such as carbon nanotubes), and nanoparticles (such as gold nanoparticles or quantum dots). The probes (nucleic acid probe such as cDNA or oligonucleotide) or antibodies and/or aptamer probes can be directly linked to the support, or attached to the surface via a linker. The solid support surface and/or the probes can be derivatived using methods known in the art to facilitate binding of the probes to the solid support, so long as the derivatization does not eliminate detection of binding between the probes and their targets. Other molecules, such as reference or control nucleic acids, proteins, antibodies, and/or aptamers can be optionally immobilized on the solid surface as well. Methods for immobilizing such molecules on a variety of solid surfaces are well known to those of skill in the art.

The probes of the methods, compositions and kits described herein are directed to specifically bind to or hybridize with genes or gene products corresponding to SEQ ID NOs: 1-141. In some embodiments, the arrays comprise a plurality of probes which specifically bind to or hybridize with at least 2, 3, 5, 10, 15, 20, 30, 40, 50, 100, 110, 120, 130, 140, 141 of the genes or gene products. In some embodiments, the arrays comprise probes directed to a subset of genes or gene products corresponding to SEQ ID Nos: 1-141. In some embodiments, the arrays consist essentially of probes directed to a subset of genes or gene products corresponding to SEQ ID Nos: 1-141. In some embodiments, the subset comprises of at least four genes selected from the group consisting of TRMU, CKAP5, PLCG2, FUS, WEE1, ITPR3, SNRPA, PKMYT1, and SUPT5H and/or at least two genes or gene products selected from group consisting of PD-L1, CTLA4, IL15RA, GNS, PTPRM, AMICA, CFH, CD2, ITGAL, ACTN1, A2M and IL2RB and/or at least three genes or gene products selected from group consisting of SPCS3, SUCLG1, NDUFAB1, FADD, MRPS16, ATP6V1D, NDUFB1, NDUFB3, SEC11A and PARK7. In some embodiments, the subset consists essentially of at least four genes or gene products selected from group consisting of TRMU, CKAP5, PLCG2, FUS, WEE1, ITPR3, SNRPA, PKMYT1, and SUPT5H and/or at least two genes or gene products selected from group consisting of PD-L1, CTLA4, IL15RA, GNS, PTPRM, AMICA, CFH, CD2, ITGAL, ACTN1, A2M and IL2RB and/or at least three genes or gene products selected from group consisting of SPCS3, SUCLG1, NDUFAB1, FADD, MRPS16, ATP6V1D, NDUFB1, NDUFB3, SEC11A and PARK7. In some embodiments, the subset consists of at least four genes or gene products selected from group consisting of TRMU, CKAP5, PLCG2, FUS, WEE1, ITPR3, SNRPA, PKMYT1, and SUPT5H and/or at least two genes or gene products selected from group consisting of PD-L1, CTLA4, IL15RA, GNS, PTPRM, AMICA, CFH, CD2, ITGAL, ACTN1, A2M and IL2RB and/or at least three genes or gene products selected from group consisting of SPCS3, SUCLG1, NDUFAB1, FADD, MRPS16, ATP6V1D, NDUFB1, NDUFB3, SEC11A and PARK7.

In addition to test probes that bind the expression products of marker genes, the arrays disclosed herein can contain a number of control probes. Non limiting examples of control probes can include: 1) probes that are complementary to labeled reference oligonucleotides or other nucleic acid sequences that are added to the nucleic acid sample; 2) probes that hybridize specifically with constitutively expressed genes in the sample such as housekeeping genes (e.g., genes set forth in Table 4) which are typically essentially invariant between samples with respect to disease stage etc. and vary only according to the number of cells in the sample; and 3) mismatch probes which are identical to their corresponding test or control probes except for the presence of one or more mismatched bases.

Nucleic acid probes of the methods, compositions and kits described herein are capable of specifically hybridizing to a target region of a polynucleotide gene product, such as for example, an RNA transcript or cDNA generated therefrom. As used herein, specific hybridization means the probe forms an anti-parallel double-stranded structure with the target region under certain hybridizing conditions, while failing to form such a structure with non-target regions or sequences when incubated with the polynucleotide under the same hybridizing conditions. The composition and length of each probe will depend on the nature of the transcript containing the target region as well as the type of assay to be performed with the probe, and is readily determined by the skilled artisan. In some embodiments, each probe of the methods, compositions and kits described herein is a perfect complement of its target region. A probe is said to be a “perfect” or “complete” complement of another nucleic acid molecule if every nucleotide of one of the molecules is complementary to the nucleotide at the corresponding position of the other molecule. While perfectly complementary probes are preferred for detecting transcripts of the Table 1 genes, departures from complete complementarity are contemplated where such departures do not prevent the molecule from specifically hybridizing to the target region as defined above. For example, an oligonucleotide probe can have one or more non-complementary nucleotides at its 5′ end or 3′ end, with the remainder of the probe being completely complementary to the target region. Alternatively, non-complementary nucleotides may be interspersed into the probe as long as the resulting probe is still capable of specifically hybridizing to the target region.

In some preferred embodiments, each probe specifically hybridizes to its target region under stringent hybridization conditions. Stringent hybridization conditions are sequence-dependent and vary depending on the circumstances. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. As the target sequences are generally present in excess, at Tm, 50% of the probes are occupied at equilibrium.

Typically, stringent conditions include a salt concentration of at least about 0.01 to 1.0 M sodium ion concentration (or other salts) at pH 7.0 to 8. 3 and the temperature is at least about 25° C. for short oligonucleotide probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations. Additional stringent conditions can be found in Molecular Cloning: A Laboratory Manual, Sambrook et al., Cold Spring Harbor Press, Cold Spring Harbor, N.Y. (1989), chapters 7, 9, and 11, and in NUCLEIC ACID HYBRIDIZATION, A PRACTICAL APPROACH, Haymes et al., IRL Press, Washington, D.C., 1985.

The nucleic acid probes can be comprised of any phosphorylation state of ribonucleotides, deoxyribonucleotides, and acyclic nucleotide derivatives, and other functionally equivalent derivatives. Alternatively, the nucleic acid probes can have a phosphate-free backbone, which can be comprised of linkages such as carboxymethyl, acetamidate, carbamate, polyamide (peptide nucleic acid (PNA) and the like (Varma, in MOLECULAR BIOLOGY AND BIOTECHNOLOGY, A COMPREHENSIVE DESK REFERENCE, Meyers, ed., pp. 6 17-20, VCH Publishers, Inc., 1995). The probes can be prepared by chemical synthesis using any suitable methodology known in the art, or can be derived from a biological sample, for example, by restriction digestion. The nucleic acid probes may contain a detectable label, according to any technique known in the art, including use of radiolabels, fluorescent labels, enzymatic labels, proteins, haptens, antibodies, sequence tags and the like. The nucleic acid probes may be manufactured and marketed as analyte specific reagents (ASRs) or may constitute components of an approved diagnostic device.

### Kits

Another aspect of the instant disclosure provides kits useful for practice of one or more methods disclosed herein. The kits described herein can be used to assay levels of gene expression of genes of SEQ ID NOs. 1-141 or a subset thereof. The kits can comprise one or more of the compositions described herein. In some embodiments, the kit comprises a plurality of probes directed to genes of SEQ ID Nos:1-141 or one or more subsets thereof. The kit can comprise, for example, cDNA probes, a cDNA array, oligonucleotide probes, an oligonucleotide array, nucleic acid primer pairs or arrays comprising said primer pairs, antibodies, aptamers, antibody arrays, or aptamer arrays. Embodiments relating to aspects encompassing probes and arrays are described above. In addition, the kit can comprise a reference or control sample, instructions for processing samples, performing the test and interpreting the results, buffers and other reagents necessary for performing the test. Kits can also contain other reagents such as hybridization buffer and reagents to detect when hybridization with a specific target molecule has occurred. Detection reagents may include biotin- or fluorescent-tagged oligonucleotides and/or an enzyme-labeled antibody and one or more substrates that generate a detectable signal when acted on by the enzyme. It will be understood by the skilled artisan that the set of probes and reagents for performing the assay will be provided in separate receptacles placed in the kit container if appropriate to preserve biological or chemical activity and enable proper use in the assay.

In other embodiments, each of the probes and all other reagents in the kit have been quality tested for optimal performance in an assay designed to quantify gene expression levels of one or more marker genes of SEQ ID Nos: 1-141 and one or more housekeeping genes selected from Table 4, in a frozen or paraffin embedded tumor section. In some embodiments, the kit includes an instruction manual that describes how to calculate a gene signature score from the quantified RNA expression levels. A kit can also include software or authorization to download and use software including computer readable instructions for analysis of the assay results, including software that permits prediction or diagnosis of DLBCL subtype(s) based on assay data. Kits can also include software or authorization to download or access and use a database including, for example, data regarding expression patterns from human or laboratory animal genes and gene fragments (corresponding to the genes of Tables 1-2).

Some embodiments of the instant application relate to kits combining, in different combinations, high-density oligonucleotide, antibody, and/or aptamer arrays, reagents for use with the arrays, signal detection and array-processing instruments, gene expression databases and analysis, manuals and database management software described above. The databases packaged with or accessible through the kits are a compilation of expression patterns from human or laboratory animal genes and gene fragments (corresponding to the genes of Tables 1-2). Data are collected from a repository of both normal and diseased animal tissues and provides reproducible, quantitative results, including, for example, the degree to which a gene is up-regulated or down-regulated under a given condition. Examples of such kit uses include kits for in situ hybridization, for PCR, for application of the NanoString technology, and for sequencing.

Analysis

Aspects of the methods described herein encompass transformation of the assayed gene expression values in order to diagnose and classify DLBCL subtype. The transformation can comprise normalization of the assayed levels using a control and identifying the genes whose assayed levels are upregulated. An Exemplary setting according to the methods can assay multiple variables e.g., expression of at least 3, 5, 10, 15, 20, 30, 40, 50, 60, 70, 80, 100, 120, 130, 140, or 141 genes to diagnose and classify DLBCL subtype. Therefore, in some embodiments, multivariate analysis is used to analyze the gene expression. “Multivariate analysis” as used herein refers to any statistical technique used to analyze data that arises from more than one variable. Suitable methods known in the art for multivariate analysis, for example, include hierarchical clustering methods (Eisen, et al., 1998), principal component analysis, principal component regression, factor analysis, partial least squares, fuzzy clustering, artificial neural networks, parallel factor analysis, Tucker models, generalized rank annihilation method, locally weighted regression, ridge regression, total least squares, principal covariates regression, Kohonen networks, linear or quadratic discriminant analysis, k-nearest neighbors based on rank-reduced distances, multilinear regression methods, soft independent modeling of class analogies, and robustified versions of the above non-linear versions each contemplated to determine the modulated genes.

### Normalization

Methods described herein can include various types of normalization of raw expression data to permit reliable sub-typing of DLBCL. Some commonly used methods for calculating a normalization factor include: (i) global normalization that uses all genes assayed (e.g., all genes assayed on an array); (ii) housekeeping gene normalization that uses constantly expressed housekeeping/invariant genes; and (iii) internal controls normalization that uses known amount of exogenous control genes added for hybridization (Quackenbush Nat. Genet. 32 (Suppl.), 496-501 (2002). In one embodiment, the marker genes disclosed herein can be normalized to control housekeeping genes.

Thus, in one embodiment, the genes in an assay are represented by a set of probes, and the RNA expression level of each of the genes is normalized by the mean or median expression level across all of the represented genes, i.e., across all marker genes and normalization genes in a gene expression assay described herein. In one embodiment, the normalization is carried out by dividing the median or mean level of RNA expression of all of the genes in the gene expression assay. In another embodiment, the RNA expression levels of the marker genes disclosed herein are normalized by the mean or median level of expression of a set of normalization genes. In one embodiment, the normalization genes comprise housekeeping genes (e.g., one or more genes selected from Table 4). In another embodiment, the normalization of a measured RNA expression level for a gene is accomplished by dividing the measured level by the median or mean expression level of the normalization genes.

### Identifying Upregulated Genes

In some embodiments the genes are identified to be upregulated upon comparison to all the genes assayed in a sample. An exemplary method for this purpose can be gene set enrichment analysis. Detailed methods for such analysis are known in the art and are described for example in Subramanian et. al. 2005; the contents of which are included herein in its entirety) and as exemplified herein. Another exemplary method can include assigning the expression level of each marker gene assayed to a percentile level of expression either higher or lower than the mean or average expression levels of all genes assayed.

In some embodiments, the genes can be identified to be upregulated upon comparison to a suitable reference. In one embodiment, the reference comprises obtaining a “reference sample” from which expression product levels are detected and compared to the expression product levels from the test sample. Such a reference sample can comprise any suitable sample, including but not limited to a sample from a control DLBCL patient (can be stored sample or previous sample measurement) with a known subtype or outcome; normal tissue or cells isolated from a subject, such as a healthy subject or the DLBCL patient, cultured primary cells/tissues isolated from a subject such as a healthy subject or the DLBCL patient, adjacent normal cells/tissues obtained from the same organ or body location of the DLBCL patient, a tissue or cell sample isolated from a healthy subject, or primary cells/tissues obtained from a depository, (for example, Novartis database depository with the GEO Accession No.: GSE1133).

In some embodiments, the reference may comprise a reference standard expression product level or an expression product level range from any suitable source e.g., from normal tissue (or other previously analyzed control sample), a previously determined expression product level range within a test sample from a group of patients (such as DLBCL patients or patients with known DLBCL subtype), or a pool of patients with a certain outcome (for example, survival for one, two, three, four years, etc.) or receiving a certain treatment (for example, CHOP or R-CHOP). In one embodiment, the reference can comprise a reference standard expression product level or an expression product level range in normal or non-cancerous cell/tissue sample. In some embodiments, the reference standard expression product level or an expression product level range is of the same gene as assayed in the test sample. In some embodiments, the upregulation is relative to the levels of gene expression of the assayed marker gene in a sample from a non-cancer cell. In some embodiments, the reference standard expression product level or an expression product level range is of a control gene, e.g., a housekeeping gene.

The sensitivity of identifying the modulated (e.g., upregulated, downregulated) genes from amongst the one or more marker genes assayed from a gene set can be increased if the expression levels of individual marker genes assayed are compared to the expression of the same genes in a pool of reference samples.

Accordingly, in some embodiments, the reference can comprise an expression level for a pool of patients, such as a pool of (e.g.) DLBCL patients, or a set of DLBCL with known DLBCL subtype, or for a pool of DLBCL patients receiving a certain treatment (e.g. CHOP or R-CHOP as discussed below) or for a pool of patients with one outcome versus another outcome. In the former case the specific expression product level of each patient can be assigned to a percentile level of expression, or expressed as either higher or lower than the mean or average of the reference standard expression level.

In some embodiments, the reference can also comprise a measured value, for example, mean or median level of expression of a particular gene in a pool of subjects compared to the level of expression of a housekeeping gene in the same pool. Such a pool can comprise normal subjects, patients with DLBCL who have not undergone any treatment (i.e., treatment naïve), DLBCL patients undergoing CHOP therapy or R-CHOP therapy, or patients with known DLBCL subtype. In some embodiments, the expression level data for all genes can be log transformed e.g., before means or medians are taken. In some embodiments, the reference comprises a ratio transformation of expression product levels, including but not limited to, determining a ratio of expression product levels of two genes in the test sample and comparing it to any suitable ratio of the same two genes in a reference standard; determining expression product levels of the two or more genes in the test sample and determining a difference in expression product levels in any suitable reference; and determining expression product levels of the two or more genes in the test sample, normalizing their expression to expression of housekeeping genes in the test sample, and comparing to any suitable reference.

In some embodiments, the reference comprises a reference sample which is of the same lineage and/or type as the test sample, e.g., the reference comprises a DLBCL tumor sample. In some embodiments, the control can comprise expression product levels grouped as percentiles within or based on a pool of patient samples, such as all patients with DLBCL. In one embodiment, a reference expression product level is established wherein higher or equal levels of expression product relative to, for instance, a particular percentile, are used as the basis for identifying modulated genes. In another embodiment, a reference expression product level is established using expression product levels from DLBCL control patients with a known DLBCL subtype, and the expression product levels from the test sample are compared to the reference expression product level as the basis for predicting modulated genes and identifying DLBCL subtype.

When comparing a subject's test sample with a reference, the expression value of a particular gene in the sample is compared to the expression value of that gene in the reference. In one embodiment, for each marker gene assayed within a selected gene set or gene signature (e.g. gene set corresponding to SEQ ID NOs 1-141 or subset thereof), the log(10) ratio is created for the expression value in the individual sample relative to the reference. A score for a selected gene set or gene signature is calculated by determining the mean log(10) ratio of the genes in the selected gene set or gene signature. If the gene signature score for the test sample is equal to or greater than a pre-determined threshold for that gene signature, then the sample is considered to be positive for the gene signature biomarker. The pre-determined threshold may also be the mean, median, or a percentile of scores for that gene signature in a collection of samples or a pooled sample used as a reference. It will be recognized by those skilled in the art that other differential expression values, besides log(10) ratio, can be used for calculating a signature score, as long as the value represents an objective measurement of transcript abundance of the genes. Examples include, but are not limited to: xdev, error-weighted log (ratio), and mean subtracted log(intensity). As demonstrated by the Examples below, the methods described herein are not limited to use of a specific cut-point in comparing the level of expression product in the test sample to the reference.

### Weighted Gene Expression

In some embodiments, the expression level of gene products of marker genes are transformed into weighted gene expression within a given gene set assayed or gene signature. “Weighted” refers to transformation of a variable such as gene expression levels of individual genes to determine the contribution of the variable. In some embodiments, identifying the modulated genes involves determining the genes with higher weights within a gene set or gene signature assayed. In some embodiments, the reference comprises gene weights derived from a computational bioinformatics analysis of the relative expression levels of at least the selected set of genes from a pool of healthy subjects or a pool of subjects with known DLBCL subtype compiled in a weighted gene expression reference database (WGERD). In certain embodiments, provided herein are weights for signature genes for all three DLBCL subtypes (exemplified in Table 1). The weights of genes provided in Table 1 can be rounded to the nearest 1/10; 1/100; 1/1,000; 1/10,000; 1/100,000; 1/1,000,000; 1/10,000,000; or 1/100,000,000. One computer-based bioinformatics algorithm used to determine the weighted values is part of the glmnet package in R computer environment (cran.us.r-project.org/). The use of this package is also is known in the art and described in examples disclosed herein. Quantification of gene expression levels and therefore weight calculations are platform and method independent. Microarray-based platforms (for instance, Affymetrix, Illumina Nimblegen chips, Nanostring), sequencing-based reactions (for instance Illumina or Roche next-generation seq or traditional Sanger seq) and any other quantitative approaches (for instance qPCR-based, such as the Fluidigm system) can be used to determine nominal gene expression levels with any of the weight calculation methods known in the art or exemplified herein.

Using methods disclosed herein, weight values can range from −1 to 1, and represent the contribution of individual genes within a gene signature. Genes with weight values closer to −1 and 1 have the highest contribution, thus importance. In some embodiments, the upregulated genes within a gene signature assayed comprises genes determined to have higher weights and thus high contribution. Weights can also be calculated using data-reduction methods that may or may not include a priori clustering steps as to the case of WGCNA (based on co-expression), using methods such as Principal Component Analysis (PCA), Multi-Dimensional Scaling (MDS), and Independent Component Analysis (ICA). Weights calculation can be extended to use biological information, such as protein-protein interactions (PPIs), gene-to-gene interactions (GGIs), Gene Ontology (GO) information, and network or ranking position; therefore both statistical and biological-based methods can be applied to derive weights from gene expression data.

The methods disclosed herein encompass diagnosing and classifying the DLBCL subtype based on identification of the upregulated marker genes. In performing comparisons to a pool, two approaches can be used. First, the expression levels of the marker genes in the sample can be compared to the expression level of those genes in the pool, where nucleic acid derived from the sample and nucleic acid derived from the pool are hybridized during the course of a single experiment, so as to identify the upregulated genes. Such an approach requires that a new pool of nucleic acid be generated for each comparison or limited numbers of comparisons, and is therefore limited by the amount of nucleic acid available. Alternatively, in some embodiments, the expression levels in a pool, whether normalized and/or transformed or not, are stored on a computer, or on computer-readable media, to be used in comparisons to the individual expression level data from the sample (i.e., single-channel data). In some embodiments, the expression levels can be stored on a computer, or a computer-readable media in form of a classifying algorithm or classifier.

In some embodiments, the identification of upregulated genes within a gene set assayed comprises applying the assayed gene expression to a classifier. A number of classifier algorithms are known in the art, for example, Elastic net algorithm (implemented in glmnet package in R), Random forest (implemented in randomforest package in R) and shrunken centroid (implemented in pamr package in R). In one embodiment, the classifier comprises a predictive computer based mathematical algorithm, developed using “training data” (e.g., gene expression data from samples of known DLBCL subtypes). The classifier can be used to classify an unknown sample (e.g. test sample) according to subtype. Methods to develop a classifier are known in the art. An exemplary method can include a training set of samples with known class or outcome used to produce a mathematical model which is then evaluated with independent validation data sets. Herein, a “training data” set of intrinsic gene expression data is used to construct a statistical model that predicts correctly the “subtype” of each sample. This training set is then tested with independent data (referred to as a test or validation set) to determine the robustness of the computer-based model. In some embodiments, the classifier has been trained in silico with a “training data” set. In some embodiments, the training data are obtained from a plurality of DLBCL patients and comprise for each DLBCL patient, weighted gene expression of a plurality of genes (e.g. genes of SEQ ID Nos 1-141 or subset thereof) and information with respect to the DLBCL subtype based on the weighted gene expression levels. The plurality of DLBCL patients includes a sufficient number of patients belonging to each DLBCL subtype, or samples derived from subjects belonging to each DLBCL subtype. By “sufficient” or “representative number” in this context is intended a quantity derived from each subtype that is sufficient for building a classification model that can reliably distinguish each subtype from all others in the group. The samples or patients for generation of a training set can be selected and subtyped, for example, using an expanded intrinsic gene set according to the methods disclosed in International Patent Publication WO 2007/061876 and US Patent Publication No. 2009/0299640, which is herein incorporated by reference in its entirety. Alternatively, the samples can be subtyped according to any known assay for classifying DLBCL subtypes. After stratifying the training samples according to subtype, an Elastic net-based prediction algorithm is used to construct weight based on the expression profile of the marker gene set described in Table 1.

Classifiers

Classifiers comprising genes as variables and accompanying weighting factors can be used to classify gene expression data. In some embodiments the classifier can be sparse linear classifiers. “Sparse”, in this context means that the vast majority of the genes measured in the expression experiment have zero weight in the final linear classifier. Sparsity ensures that the sufficient and necessary gene lists produced by the methodology described herein are as short as possible. These short weighted gene lists (i.e., a gene signature) are capable of predicting a DLBCL subtype based on the weighted gene signature identified.

The sparsity and linearity of the classifiers are important features. The linearity of the classifier facilitates the interpretation of the signature. As it relates to the instant disclosure, the contribution of each marker gene assayed to the classifier can correspond to the product of its weight and the value (i.e., log 10 ratio) from the gene expression experiment. The property of sparsity ensures that the classifier uses only a few genes, which also helps in the interpretation. More importantly, the sparsity of the classifier can be reduced to a practical diagnostic apparatus or device comprising a relatively small set of reagents representing genes.

In some embodiments, the classifier is a linear classifier, used to generate one or more gene signatures capable of answering a classification question comprising a series of genes and associated weighting factors. These signatures are particularly useful because they are easily incorporated into a wide variety of DNA- or protein-based diagnostic assays (e.g., DNA microarrays). However, some classes of non-linear classifiers, so called kernel methods, can also be used to develop short gene lists, weights and algorithms that can be used in diagnostic device development; while the preferred embodiment described here uses linear classification methods, it is specifically contemplated that non-linear methods may also be suitable.

Additional statistical techniques, or algorithms, are known in the art for generating classifiers. Some algorithms produce linear classifiers, which are convenient in many diagnostic applications because they can be represented as a weighted list of variables. In other cases non-linear classifier functions of the initial variables can be used. Other types of classifiers include decision trees and neural networks. Neural networks are universal approximators (Hornik, K., M. Stinchcombe, and H. White. 1989. “Multilayer feedforward networks are universal approximators,” Neural Networks 2: 359-366); they can approximate any measurable function arbitrarily well, and they can readily be used to model classification functions as well. They perform well on several biological problems, e.g., protein structure prediction, protein classification, and cancer classification using gene expression data (see, e.g., Bishop, C. M. 1996. Neural Networks for Pattern Recognition. Oxford University Press; Khan, J., J. S. Wei, M. Ringner, L. H. Saal, M. Ladanyi, F. Westermann, F. Berthold, M. Schwab, C. R. Antonescu, C. Peterson, and P. S. Meltzer. 2001. Classification and diagnostic prediction of cancers using gene expression profiling and artificial neural networks. Nat Med 7: 673-679; Wu, C. H., M. Berry, S. Shivakumar, and J. McLarty. 1995. Neural networks for full-scale protein).

### Computer Based Media

The methods described herein can be implemented and/or the results recorded using any device capable of implementing the methods and/or recording the results. Non-limiting examples of devices that can be used include, but are not limited to, electronic computational devices, including computers of all types. When the methods described herein are implemented and/or recorded in a computer, the computer program that can be used to configure the computer to carry out the steps of the methods can be contained in any computer readable medium capable of containing the computer program. Examples of computer readable medium that can be used include but are not limited to diskettes, CD-ROMs, DVDs, ROM, RAM, and other non-transitory memory and computer storage devices. The computer program that can be used to configure the computer to carry out the steps of the methods and/or record the results may also be provided over an electronic network, for example, over the internet, an intranet, or other network. The computer readable medium or a computer program product can comprise a classifier that predicts a DLBCL subtype based on the gene expression levels of the marker genes disclosed herein. The classifier is described above. The kits disclosed herein can further comprise a computer readable medium comprising instructions sufficient to direct a computer to perform the computational steps necessary to provide a DLBCL subtype based on target gene expression analysis as described herein.

### Diagnosis and Classification of DLBCL

The methods and compositions described herein encompass assaying expression levels of genes of SEQ ID NO: 1-141 or a subset thereof to provide diagnostic information. As used herein the term “diagnostic information” includes, but is not limited to, any type of information that is useful in determining whether a patient has, or is at increased risk for developing, a disease or disorder; for providing a prognosis for a patient having a disease or disorder; for classifying a disease or disorder; for monitoring a patient for recurrence of a disease or disorder; for selecting a preferred therapy; for predicting the likelihood of response to a therapy, etc. In some embodiments, the method and compositions are used for providing diagnostic information for cancer. In some embodiments, the cancer is DLBCL or a particular subtype thereof.

In one aspect, provided herein are methods for diagnosing cancer (e.g., DLBCL or specific subtype thereof) in a subject having or suspected of having DLBCL. The method comprises assaying a suitable sample (e.g., from a tumor) from the subject for expression of a set of marker genes disclosed herein and diagnosing the subject as suffering from DLBCL of the BCR-subtype, if the expression of at least four genes of the group consisting of TRMU, CKAP5, PLCG2, FUS, WEE 1, ITPR3, SNRPA, PKMYT1, and SUPT5H are identified to be upregulated; or diagnosing the subject as suffering from DLBCL of the Host Receptor subtype, if the expression of at least two genes of the group consisting of PD-L1, CTLA4, IL15RA, GNS, PTPRM, AMICA, CFH, CD2, ITGAL, ACTN1, A2M and IL2RB are identified to be upregulated; or diagnosing the subject as suffering from DLBCL of the OxPhos subtype, if the expression of at least three genes of the group consisting of SPCS3, SUCLG1, NDUFAB1, FADD, MRPS16, ATP6V1D, NDUFB1, NDUFB3, SEC11A and PARK7 are identified to be upregulated.

It is well known in the art that different tumor subtypes can respond to different therapies. Many compounds have been tested for anti-tumor activity and appear to be effective in only a small percentage of tumors. The current inability to readily identify DLBCL tumor subtypes makes it difficult to reliably select an agent more likely to be effective than others. However, the methods and compositions disclosed herein offer the ability to identify tumor subtypes characterized by a significant likelihood of response to a given agent. In an approach that can expand the number of subtype specific agents effective for treatment of DLBCL, one can examine existing tumor archives. Tumor sample archives containing tissue samples obtained from patients that have undergone therapy with various agents are available along with information regarding the results of such therapy. In general such archives include of tumor samples embedded in paraffin blocks. These tumor samples can be analyzed for their expression of genes of SEQ ID NOs 1-141 or a subset thereof or polypeptides encoded by the said genes. For example, immunohistochemistry can be performed using antibodies that bind to the polypeptides. Methods for analysis of nucleic acids in formalin fixed, paraffin embedded samples are also known. Tumors belonging to the specific DLBCL subtype can then be identified on the basis of this information using methods described herein. It is then possible to correlate the expression of the genes with the response of the tumor to therapy, thereby identifying particular compounds that show a superior efficacy in tumors of this subtype as compared with their efficacy in tumors overall or in tumors not falling within a particular subtype. Once such compounds are identified, it will be possible to select patients whose tumors fall into a particular DLBCL subtype for additional clinical trials using these compounds. Such clinical trials, performed on a selected group of patients, are more likely to demonstrate efficacy. The compositions and methods provided herein, therefore, are valuable both for retrospective and prospective trials.

In one aspect, detection of expression products of one or more of the marker genes can be used to stratify patients prior to their entry into a trial for subtype targeted therapy or while they are enrolled in the trial. In clinical research, stratification is the process or result of describing or separating a patient population into more homogeneous subpopulations according to specified criteria. Stratifying patients initially rather than after the trial is frequently preferred, e.g., by regulatory agencies such as the U.S. Food and Drug Administration that may be involved in the approval process for a medication. In some cases stratification can be required by the study design. Various stratification criteria may be employed in conjunction with detection of expression of one or more marker genes. Commonly used criteria include age, family history, tumor size, tumor grade, etc. Other criteria including, but not limited to, tumor aggressiveness, prior therapy received by the patient, various other biomarkers, etc., can also be used. Stratification is frequently useful in performing statistical analysis of the results of a trial. Ultimately, once compounds that exhibit superior efficacy against a specific DLBCL tumor subtype are identified, reagents for detecting expression of the marker genes as described herein can be used to guide the selection of appropriate chemotherapeutic agent(s). Accordingly, by providing reagents and methods for classifying tumors based on their expression of the marker genes, the present disclosure provides an avenue to individualize therapy. The disclosure further provides a means to identify a patient population that can benefit from potentially promising therapies that have been abandoned due to inability to identify the patients who would benefit from their use.

Information regarding the expression of the marker genes disclosed herein is useful even in the absence of specific information regarding their biological function or role in tumor development, progression, maintenance, or response to therapy.

In general, the results of such a diagnostic test can be presented in any of a variety of formats. The results can be presented in a qualitative fashion. For example, the test report may indicate only whether or not a gene product for a particular marker gene was detected, perhaps also with an indication of the limits of detection. The results may be presented in a semi-quantitative fashion. For example, various ranges may be defined, and the ranges may be assigned a score (e.g., 1+ to 4+) that provides a certain degree of quantitative information. Such a score may reflect various factors, e.g., the number of cells in which the gene product is detected, the intensity of the signal (which may indicate the level of expression of the polypeptide), etc. The results may be presented in a quantitative fashion, e.g., as a percentage of cells in which the gene product is detected, as a protein concentration, etc. In some embodiments, the output is in form of a score reflecting a diagnosis for a particular subtype of DLBCL.

The diagnosis and classification of DLBCL and subtype thereof in accordance with embodiments disclosed herein can be used in combination with any other effective classification feature or set of features. For example, a disorder can be classified by methods disclosed herein in conjunction with WHO suggested guidelines, morphological properties, histochemical properties, chromosomal structure, genetic mutation, cellular proliferation rates, immunoreactivity, clinical presentation, and/or response to chemical, biological, or other agents. Embodiments disclosed herein can be used in lieu of or in conjunction with other methods for lymphoma diagnosis, such as immunohistochemistry, flow cytometry, FISH for translocations, or viral diagnostics.

Accurate determination of lymphoma type (e.g., DLBCL type) in a subject allows for better selection and application of therapeutic methods. Knowledge about the exact lymphoma affecting a subject allows a clinician to select therapies or treatments that are most appropriate and useful for that subject, while avoiding therapies that are nonproductive or even counterproductive. The diagnostic and identification methods described herein allow for more precise delineation between these lymphomas, which simplifies the decision of whether to pursue a particular therapeutic option.

### Treatment of Cancer

Provided herein are methods for treatment of cancer, treatment of DLBCL and specific DLBCL subtypes. The methods in general comprise assaying a sample from a subject for expression levels of one or more marker genes disclosed herein and administering a therapeutic treatment targeting a particular DLBCL subtype upon identification of modulation (e.g., upregulation) of genes indicative of a specific subtype. In some embodiments, the subject is administered a therapeutic treatment for the BCR subtype if the expression of at least *______* of the group consisting of TRMU, CKAP5, PLCG2, FUS, WEE1, ITPR3, SNRPA, PKMYT1, and SUPT5H are identified to be upregulated, a therapeutic regimen for Host Receptor subtype if the expression of at least *______* genes of the group consisting of PD-L1, CTLA4, IL15RA, GNS, PTPRM, AMICA, CFH, CD2, ITGAL, ACTN1, A2M and IL2RB are identified to be modulated or therapeutic treatment for OxPhos subtype if the expression of at least *______* of the group consisting of SPCS3, SUCLG1, NDUFAB1, FADD, MRPS16, ATP6V1D, NDUFB1, NDUFB3, SEC11A and PARK7 are identified to be modulated.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a cancer (e.g., tumor size), diminishment of extent of a cancer or tumor, stabilized (i.e., not worsening) cancer, delay or slowing of progression of the disease, amelioration or palliation of the disease state, and remission (whether partial or total). The term “treatment” of a disease also includes providing at least partial relief from the symptoms or side-effects of the disease (including palliative treatment).

In one embodiment, as used herein, the term “prevention” or “preventing” when used in the context of a subject refers to stopping, hindering, and/or slowing the development of a cancer or metastasis from a primary or a secondary tumor.

As used herein, the term “therapeutically effective amount” means that amount necessary, at least partly, to attain the desired effect, or to delay the onset of, inhibit the progression of, or halt altogether, the onset or progression of the particular disease or disorder being treated (e.g., DLBCL or a subtype thereof). Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. In some embodiments, a maximum dose of a therapeutic agent is used, that is, the highest safe dose according to sound medical judgment. It will be understood by those of ordinary skill in the art, however, that a lower dose or tolerable dose that is effective can be administered for medical reasons, psychological reasons or for other reasons.

In one embodiment, a therapeutically effective amount of a pharmaceutical formulation, or a composition for a method of treating cancer is an amount sufficient to reduce the level of at least one symptom of cancer (e.g., (1) reduction in tumor size; (2) decrease in cell number; (3) increase in the tumor apoptosis; (4) inhibition of tumor cell survival; (4) inhibition of tumor growth (i.e., a degree of slowing, preferably stopping); (5) inhibition of cancer cell infiltration into peripheral organs (that is, slowing to some extent, preferably stopping); (6) inhibition of tumor metastasis (that is, slowing to some extent, preferably stopping); (7) the inhibition of tumor growth; (8) an increased patient survival rate; and (9) some extent of relief of one or more symptoms associated with DLBCL) as compared to the level in the absence of the therapeutic treatment. In other embodiments, the amount of the composition administered is preferably safe and sufficient to treat or delay the development of cancer, and/or delay onset of the disease. In some embodiments, the amount can thus cure or result in amelioration of the symptoms of cancer, slow the course of the disease, slow or inhibit a symptom of the disease, or slow or inhibit the establishment or development of secondary symptoms of a cancer. An effective amount of a composition described herein can inhibit further symptoms associated with a cancer or cause a reduction in one or more symptoms associated with a cancer. While effective treatment need not necessarily initiate complete regression of the disease, such effect would be effective treatment. The effective amount of a given therapeutic agent will vary with factors such as the nature of the agent, the route of administration, the size and species of the animal to receive the therapeutic agent, and the purpose of the administration. Thus, it is not possible or prudent to specify an exact “therapeutically effective amount.” However, for any given case, an appropriate “effective amount” can be determined by a skilled artisan according to established methods in the art using only routine experimentation.

In some embodiments, the treatment includes one or more of the standard treatments of DLBCL (e.g., CHOP). or one or more of novel drugs for DLBCL under clinical evaluation (described, for example in Camicia et al. Molecular Cancer (2015) 14:207, the contents of which are incorporated herein in its entirety). The standard treatment of DLBCL is chemotherapy based on CHOP. CHOP consists of four chemotherapy drugs—Cyclophosphamide (also called Cytoxan/Neosar), Doxorubicin (also called Hydroxydaunorubicin) (or Adriamycin), Vincristine (Oncovin) and Prednisolone. The development of new treatment regimens including M-BACOD (methotrexate, bleomycin, doxorubicin, cyclophosphamide, vincristine, and dexamethasone), MACOP-B (methotrexate with leucovorin rescue doxorubicin, cyclophosphamide, vincristine, prednisone, and bleomycin) and ProMACE/CytaBOM (cyclophosphamide, doxorubicin, etoposide cytozar, bleomycin, vincristine, methotrexate prednisone) were reported to achieve results that seemed much better than had been observed with CHOP. The CHOP therapy was expanded to a combination of chemotherapy and immunotherapy, i.e. R-CHOP. R—CHOP is a combination of drugs used in chemotherapy for aggressive Non-Hodgkin Lymphomas (NHL). It adds the drug Rituximab—a monoclonal antibody against CD20, to the standard combination called CHOP.

A commonly applied R-CHOP treatment regime is as follows: Rituximab is administered as an infusion over a few hours on the first day of treatment, while the drugs of the CHOP regimen may be started the next day. The entire course is usually repeated every three weeks for 6-8 cycles. The first three drugs of the CHOP chemotherapy regimen are usually given as injections or infusions in veins on a single day, while prednisolone is taken as pills for five days. Each cycle is repeated every 3 weeks for 6-8 cycles. CHOP chemotherapy is used for many of the common types of aggressive Non-Hodgkin Lymphomas including Diffuse Large B-Cell Lymphoma (DLBCL). Nowadays, R—CHOP can be considered the standard first line treatment for patients with DLBCL. Various other regimens are available such as R-DHAP (rituximab, dexamethasone, high-dose cytarabine, and cisplatin) R-DHAP-VIM-DHAP (rituximabcisplatin), cytarabine, dexamethasone, etoposide—ifosfamide, methotrexate—cisplatin, cytarabine, dexamethasone), R-ESHAP (rituximab, etoposide, steroids, ara-C, and cisplatin), R-DHAX (rituximab, dexamethasone, cytarabine, and oxaliplatin), R-ICE (rituximab, ifosfamide, carboplatin, etoposide), DA-EPOCH-R (etoposide, doxorubicin, and cyclophosphamide with vincristine, prednisone, and rituximab), R-GIFOX (rituximab, gemcitabine, ifosfamide, oxaliplatin), R-GEMOX (rituximab, emcitabine, oxaliplatin), R-GDP (rituximab plus gemcitabine, cisplatin, and dexamethasone), R-MINE (rituximab mesna, ifosfamide, mitoxantrone, etoposide) or R-BEAM (rituximab plus carmustine, etoposide, cytarabine, and melphala).

The methods described herein, can be used to treat primary, relapsed, transformed, or refractory forms of cancer (e.g., DLBCL or subtype thereof). To detect or identify a refractory cancer, patients undergoing chemotherapy treatment can be carefully monitored for signs of resistance, non-responsiveness or recurring cancer. The response, lack of response, or relapse of the cancer to the initial treatment can be determined by imaging and diagnostic methods used in the art to evaluate tumor load and tumor growth. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” (Response Evaluation Criteria In Solid Tumors) criteria as described in detail in Therasse et al, J. Natl. Cancer Inst., 92:205-216 (2000). Accordingly, the methods and compositions described herein can be used to treat DLBCL which has been found to be refractory to one or more of the standard DLBCL treatments (e.g. CHOP) or relapsed post treatment with standard DLBCL therapeutic options. Often, patients with relapsed cancers have undergone one or more treatments including chemotherapy, radiation therapy, bone marrow transplants, hormone therapy, surgery, and the like. Of the patients who respond to such treatments, they may exhibit stable disease, a partial response (i.e., the tumor or a cancer marker level diminishes by at least 50%), or a complete response (i.e., the tumor as well as markers become undetectable). In either of these scenarios, the cancer may subsequently reappear, signifying a relapse of the cancer. In certain embodiments, the methods provided herein will be used to treat a patient that has undergone a single course of treatment for a cancer, has partially or completely responded to such treatment, and has subsequently suffered a relapse. In other embodiments, patients are treated who have undergone more than one course of treatment, have responded more than once, and have subsequently suffered more than one relapse. The previous course of treatment can include any anti-cancer treatment, including chemotherapy, radiation therapy, bone marrow transplant, standard treatment for DLBCL described above etc.

Therapeutic compositions comprising previously known or novel agents (as described above and e.g., targeting one or more marker genes disclosed herein) directed for treatment of cancer (e.g., DLBCL or subtype thereof) are encompassed in the present disclosure. The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions. Formulations suitable for parenteral administration can be formulated, for example, for intravenous, intramuscular, intradermal, intraperitoneal, and subcutaneous routes. Carriers can include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

Therapeutic compositions contain a physiologically tolerable carrier together with an active agent as described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmaceutical composition that contains active ingredients dissolved or dispersed therein is well understood in the art and need not be limited based on formulation. Typically such compositions are prepared as injectable either as liquid solutions or suspensions; however, solid forms suitable for solution, or suspension in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness or stability of the active ingredient. A therapeutic composition useful in the methods described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

While any suitable carrier known to those of ordinary skill in the art can be employed in the pharmaceutical compositions described herein, the type of carrier will vary depending on the mode of administration. Compositions useful in the therapeutic methods described herein can be formulated for any appropriate manner of administration, including for example, topical, oral, nasal, intravenous, intracranial, intraperitoneal, subcutaneous or intramuscular administration. For parenteral administration, such as intramuscular or subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, can be employed. Biodegradable microspheres (e.g., polylactate polyglycolate) can also be employed as carriers for the pharmaceutical compositions. Suitable biodegradable microspheres are disclosed, for example, in U.S. Pat. Nos. 4,897,268 and 5,075,109. Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions useful in the therapeutic methods described herein can be formulated as a lyophilizate. Compounds can also be encapsulated within liposomes using well known technology. The compositions useful in the therapeutic methods can be administered as part of a sustained release formulation (i.e., a formulation such as a capsule or sponge that affects a slow release of compound following administration). Such formulations can generally be prepared using well known technology and administered by, for example, oral, rectal or subcutaneous implantation, or by implantation at the desired target site. Sustained-release formulations can contain a polypeptide, polynucleotide dispersed in a carrier matrix and/or contained within a reservoir surrounded by a rate controlling membrane. Carriers for use within such formulations are biocompatible, and can also be biodegradable; preferably the formulation provides a relatively constant level of active component release. The amount of active compound contained within a sustained release formulation depends upon the site of implantation, the rate and expected duration of release and the nature of the condition to be treated or prevented.

### Dosage and Administration

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human. The dosage range for the agent depends upon the potency, and includes amounts large enough to produce the desired effect, e.g., reduction in at least one symptom of cancer. The dosage should not be so large as to cause unacceptable adverse side effects. Generally, the dosage will vary with the type of inhibitor (e.g., an antibody or fragment, small molecule, siRNA, etc.) and with the age, condition, and sex of the patient. The dosage can be determined by one of skill in the art and can also be adjusted by the individual physician in the event of any complication. Typically, the dosage for a given drug or agent in a therapeutic regimen ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example, but not limited to, three times a day. In another embodiment, the doses recited above are administered daily for several weeks or months. The duration of treatment depends upon the subject's clinical progress and responsiveness to therapy. Continuous, relatively low maintenance doses are contemplated after an initial higher therapeutic dose. A therapeutically effective amount is an amount of an agent that is sufficient to produce a statistically significant, measurable change in at least one symptom of a cancer (see “Efficacy Measurement” below). Such effective amounts can be gauged in clinical trials as well as animal studies for a given agent.

Agents useful in the methods and compositions described herein can be administered systemically or can be administered orally. It is also contemplated herein that the agents can also be delivered intravenously (by bolus or continuous infusion), by inhalation, intranasally, intraperitoneally, intramuscularly, subcutaneously, intracavity, and can be delivered by peristaltic means, if desired, or by other means known by those skilled in the art. In some embodiments, the pharmaceutically acceptable formulation used to administer the active compound provides sustained delivery, such as “slow release” of the active compound to a subject. For example, the formulation can deliver the agent or composition for at least one, two, three, or four weeks after the pharmaceutically acceptable formulation is administered to the subject. Preferably, a subject to be treated in accordance with the methods described herein is treated with the active composition for at least 30 days (either by repeated administration or by use of a sustained delivery system, or both).

As used herein, the term “sustained delivery” is intended to include continual delivery of the composition in vivo over a period of time following administration, preferably at least several days, a week, several weeks, one month or longer.

Preferred approaches for sustained delivery include use of a polymeric capsule, a minipump to deliver the formulation, a biodegradable implant, or implanted transgenic autologous cells (as described in U.S. Pat. No. 6,214,622). Implantable infusion pump systems (such as Infusaid; see such as Zierski, J. et al, 1988; Kanoff, R. B., 1994) and osmotic pumps (sold by ALZA CORPORATION) are available in the art. Another mode of administration is via an implantable, externally programmable infusion pump. Suitable infusion pump systems and reservoir systems are described in U.S. Pat. No. 5,368,562 by Blomquist and U.S. Pat. No. 4,731,058 by Doan, developed by Pharmacia Deltec Inc.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle. The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., tumor site). Antibody-based or non-antibody-based targeting moieties can be employed to deliver a ligand or the inhibitor to a target site. Preferably, a natural binding agent for an unregulated or disease associated antigen is used for this purpose. Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood or skeletal muscle tissue in the ranges specified for in vivo therapies are contemplated.

### Efficacy Measurement

The efficacy of a given treatment for a cancer as described herein can be determined by the skilled clinician. However, a treatment is considered “effective treatment,” as the term is used herein, if any one or all of the signs or symptoms of a cancer is/are altered in a beneficial manner (e.g., reduced tumor load, slowing of tumor growth etc.), other clinically accepted symptoms or markers of disease are improved, or ameliorated, e.g., by at least 10% following treatment as described herein. Efficacy can also be measured by failure of an individual to worsen as assessed by stabilization of the cancer, hospitalization or need for medical interventions (i.e., progression of the disease is halted or at least slowed). Methods of measuring these indicators are known to those of skill in the art and/or described herein.

The methods and compositions described herein can be adapted to a companion diagnostic for any of the subtype-selective or subtype-preferred therapies for the BCR, OxPhos and/or HR subtypes of DLBCL. Alternatively, or in addition, the methods and compositions described herein can be used to monitor the progress of a selected therapy. In brief, the same combination of probes indicative of the BCR, OxPhos or HR subtype DLBCL used in diagnostic and/or selection of a subtype-selective or subtype preferred therapy can be measured after treatment is initiated, e.g., one week, two weeks, three weeks, one month or more after a given treatment, to determine whether the therapy is changing the marker profile. It is contemplated herein that a reduction or change in one or more markers indicative of a given subtype of tumor can predict or be indicative of therapeutic effectiveness.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the invention. Further, all patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:
1. A method of treating a subject for DLBCL, the method comprising: (a) assaying a sample from a cancer cell of the subject, for levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least two genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC11A and PARK7; (b) normalizing the assayed levels of gene expression with a control; (c) identifying the genes whose assayed levels of expression are upregulated; (d) administering a therapeutic regimen for the treatment of DLBCL of the BCR subtype if the expression of at least two of the group consisting of TRMU, CKAP5, PLCG2, FUS, WEE1, ITPR3, SNRPA, PKMYT1, and SUPT5H are identified to be upregulated; or (e) administering a therapeutic regimen for the treatment of DLBCL of the Host Receptor subtype if the expression of at least two genes of the group consisting of PD-L1, CTLA4, IL15RA, GNS, PTPRM, AMICA, CFH, CD2, ITGAL, ACTN1, A2M and IL2RB are identified to be upregulated; or (f) administering a therapeutic regimen for the treatment of DLBCL of the OxPhos subtype if the expression of at least two of the group consisting of SPCS3, SUCLG1, NDUFAB1, FADD, MRPS16, ATP6V1D, NDUFB1, NDUFB3, SEC11A and PARK7 are identified to be upregulated.
2. The method of paragraph 1, wherein the gene expression is assayed by measuring the nucleic acid encoded by the gene or by measuring or detecting a protein encoded by the gene.
3. The method of paragraph 2, wherein the levels of gene expression are assayed by measuring the nucleic acid encoded by the gene using qPCR, microarray, or nCounter® analysis system.
4. The method of paragraph 3, wherein the microarray is a cDNA array or an oligonucleotide array.
5. The method of paragraph 2, wherein the levels of gene expression are assayed by measuring or detecting a protein encoded by the gene using immunoassay, targeted mass spectrometry, or immunolabeling.
6. The method of any one of paragraphs 1-5, wherein step (c) is by linear combination of the normalized levels of gene expression obtained from step (b).
7. The method of paragraph 6, wherein the linear combination is a combination of weighted gene expression levels.
8. The method of any one of paragraphs 1-7, wherein step (c) comprises applying a classifier, wherein the classifier has been trained with training data from a plurality of DLBCL patients, wherein the training data comprise for each of the plurality of DLBCL patients (a) weighted gene expression level of at least the plurality of genes for which the expression levels are assayed including said at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7 and (b) information with respect to subtype of DLBCL based on the weighted gene expression level.
9. The method of paragraph 8, wherein the classifier is selected from Elastic net, Random Forest, and Shrunken centroids.
10. The method of any one of paragraphs 1-5, wherein the upregulation is relative to the levels of gene expression in a sample from a non-cancer cell.
11. The method of any one of paragraphs 1-10, wherein the subject is human.
12. The method of any one of paragraphs 1-11, wherein the DLBCL is a relapsed cancer.
13. The method of any one of paragraphs 1-12, wherein the DLBCL is refractory to treatment with a CHOP or rituximab(R)/CHOP treatment regimen.
14. The method of any one of paragraphs 1-13, wherein the cancer cell is a cell obtained from tumor biopsy, frozen cancer tissue, or paraffin-embedded cancer tissue.
15. The method of any one of paragraphs 1-14, wherein the control in step c is the expression level of a housekeeping gene.
16. The method of paragraph 15, wherein the housekeeping gene is selected from genes corresponding to those listed in Table 4.
17. A method for stratifying a subject for subtype-targeted therapy for DLBCL, the method comprising: (a) assaying a sample from a cancer cell of the subject, for levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC 11A and PARK7; (b) normalizing the assayed levels of gene expression with a control; (c) identifying the genes whose levels of expression are upregulated; (d) stratifying the subject for the clinical trial comprising BCR subtype targeted therapy, if the expression of at least two genes of the group consisting of TRMU, CKAP5, PLCG2, FUS, WEE1, ITPR3, SNRPA, PKMYT1, and SUPT5H are identified to be upregulated; or (e) stratifying the subject for the clinical trial comprising Host Receptor subtype targeted therapy, if the expression of at least two genes of the group consisting of PD-L1, CTLA4, IL15RA, GNS, PTPRM, AMICA, CFH, CD2, ITGAL, ACTN1, A2M and IL2RB are identified to be upregulated; or (f) stratifying the subject for the clinical trial comprising OxPhos subtype targeted therapy, if the expression of at least two genes of the group consisting of SPCS3, SUCLG1, NDUFAB1, FADD, MRPS16, ATP6V1D, NDUFB1, NDUFB3, SEC 11A and PARK7 are identified to be upregulated.
18. A method for diagnosing a DLBCL subtype in a subject having or suspected of having DLBCL, the method comprising: (a) assaying a sample from the subject, for levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC 11A and PARK7; (b) normalizing the assayed levels of gene expression with a control; (c) identifying the genes whose levels of expression are upregulated; (d) diagnosing the subject as suffering from DLBCL of the BCR-subtype, if the expression of at least four genes of the group consisting of TRMU, CKAP5, PLCG2, FUS, WEE1, ITPR3, SNRPA, PKMYT1, and SUPT5H are identified to be upregulated; or (e) diagnosing the subject as suffering from DLBCL of the Host Receptor subtype, if the expression of at least {two} genes of the group consisting of PD-L1, CTLA4, IL15RA, GNS, PTPRM, AMICA, CFH, CD2, ITGAL, ACTN1, A2M and IL2RB are identified to be upregulated; or (f) diagnosing the subject as suffering from DLBCL of the OxPhos subtype, if the expression of at least three genes of the group consisting of SPCS3, SUCLG1, NDUFAB1, FADD, MRPS16, ATP6V1D, NDUFB1, NDUFB3, SEC11A and PARK7 are identified to be upregulated.
19. A method of classifying a sample from a subject having or suspected of having DLBCL, the method comprising; (a) assaying a sample from the subject, for levels of gene expression of 141 genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC 11A and PARK7; (b) normalizing the assayed levels of gene expression with a control; (c) identifying the genes whose levels of expression are upregulated; (d) classifying the sample as corresponding to BCR-subtype of DLBCL, if the expression of at least four genes of the group consisting of TRMU, CKAP5, PLCG2, FUS, WEE1, ITPR3, SNRPA, PKMYT1, and SUPT5H are identified to be upregulated; or (e) classifying the sample as corresponding to Host Receptor subtype of DLBCL, if the expression of at least {two} genes of the group consisting of PD-L1, CTLA4, IL15RA, GNS, PTPRM, AMICA, CFH, CD2, ITGAL, ACTN1, A2M and IL2RB are identified to be upregulated; or (f) classifying the sample as corresponding to OxPhos subtype of DLBCL, if the expression of at least three genes of the group consisting of SPCS3, SUCLG1, NDUFAB1, FADD, MRPS16, ATP6V1D, NDUFB1, NDUFB3, SEC11A and PARK7 are identified to be upregulated.
20. A composition comprising an array comprising probes directed to genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, or at least {two} genes selected from group consisting of PD-L1, CTLA4, IL15RA, GNS, PTPRM, AMICA, CFH, CD2, ITGAL, ACTN1, A2M and IL2RB, or at least three genes selected from group consisting of SPCS3, SUCLG1, NDUFAB1, FADD, MRPS16, ATP6V1D, NDUFB1, NDUFB3, SEC11A and PARK7.
21. A composition comprising an array consisting essentially of probes directed to genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, or at least {two} genes selected from group consisting of PD-L1, CTLA4, IL15RA, GNS, PTPRM, AMICA, CFH, CD2, ITGAL, ACTN1, A2M and IL2RB, or at least three genes selected from group consisting of SPCS3, SUCLG1, NDUFAB1, FADD, MRPS16, ATP6V1D, NDUFB1, NDUFB3, SEC 11A and PARK7.
22. A composition comprising an array comprising probes directed to genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC 11A and PARK7.
23. The composition of paragraph 20, wherein the array consists essentially of probes directed to genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7.
24. The composition of paragraph 21, wherein the array consists essentially of probes directed to genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7.
25. The composition of any one of paragraphs 20-24, wherein the probes are cDNA probes.
26. A kit comprising a plurality of probes for determining levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, or at least {two} genes selected from group consisting of PD-L1, CTLA4, IL15RA, GNS, PTPRM, AMICA, CFH, CD2, ITGAL, ACTN1, A2M and IL2RB, or at least three genes selected from group consisting of SPCS3, SUCLG1, NDUFAB1, FADD, MRPS16, ATP6V1D, NDUFB1, NDUFB3, SEC 11A and PARK7.
27. A kit consisting essentially of a plurality of probes for determining levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, or at least {two} genes selected from group consisting of PD-L1, CTLA4, IL15RA, GNS, PTPRM, AMICA, CFH, CD2, ITGAL, ACTN1, A2M and IL2RB, or at least three genes selected from group consisting of SPCS3, SUCLG1, NDUFAB1, FADD, MRPS16, ATP6V1D, NDUFB1, NDUFB3, SEC 11A and PARK7.
28. A kit comprising a plurality of probes for determining levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC 11A and PARK7.
29. The kit of paragraph 26 consisting essentially of a plurality of probes for determining levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7.
30. The kit of paragraph 27 consisting essentially of a plurality of probes for determining levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7.
31. The kit of any one of paragraphs 26-30, wherein the probes are nucleic acid primers for amplification of the genes.
32. The kit of any one of paragraphs 26-31, wherein each probe in the plurality of probes comprises a target specific sequence that hybridizes to no more than one gene under stringent hybridization conditions.
33. The kit of any one of paragraphs 26-3 1, wherein the plurality of probes comprises probe pairs to detect the expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC11A and PARK7, wherein each probe in the probe pair comprises a target specific sequence that hybridizes to no more than one gene under stringent hybridization conditions, and wherein the target-specific sequences in each pair hybridize to different regions of the same gene.
34. The kit of any one of paragraphs 26-33, wherein a probe molecule for each gene comprises a label.
35. The kit of any one of paragraphs 26-34, wherein the probes are immobilized on a solid support.
36. A kit for determining levels of gene expression of genes of SEQ ID NOs 1-141 or a subset thereof, the kit comprising antibodies or antigen binding portions thereof that specifically bind polypeptides encoded by the respective genes or subset thereof, wherein the subset comprises at least four genes selected from group consisting of TRMU, CKAP5, PLCG2, FUS, WEE1, ITPR3, SNRPA, PKMYT1, and SUPT5H, or at least {two} genes selected from group consisting of PD-L1, CTLA4, IL15RA, GNS, PTPRM, AMICA, CFH, CD2, ITGAL, ACTN1, A2M and IL2RB, or at least three genes selected from group consisting of SPCS3, SUCLG1, NDUFAB1, FADD, MRPS16, ATP6V1D, NDUFB1, NDUFB3, SEC 11A and PARK7, wherein each antibody or antigen-binding portion thereof specifically binds to a protein expressed by one of the genes.
37. A kit for determining levels of gene expression of genes of SEQ ID NOs 1-141 or a subset thereof, the kit consisting essentially of antibodies or antigen binding portions thereof that specifically bind polypeptides encoded by the respective genes or subset thereof, wherein the subset comprises at least four genes selected from group consisting of TRMU, CKAP5, PLCG2, FUS, WEE1, ITPR3, SNRPA, PKMYT1, and SUPT5H, or at least {two} genes selected from group consisting of PD-L1, CTLA4, IL15RA, GNS, PTPRM, AMICA, CFH, CD2, ITGAL, ACTN1, A2M and IL2RB, or at least three genes selected from group consisting of SPCS3, SUCLG1, NDUFAB1, FADD, MRPS16, ATP6V1D, NDUFB1, NDUFB3, SEC11A and PARK7, wherein each antibody or antigen-binding portion thereof specifically binds to a protein expressed by one of the genes.
38. A kit for determining levels of gene expression of genes of SEQ ID NOs 1-141 or a subset thereof, the kit comprising antibodies or antigen binding portions thereof that specifically bind polypeptides encoded by the respective genes or subset thereof, wherein the subset comprises at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC 11A and PARK7, wherein each antibody or antigen-binding portion thereof specifically binds to a protein expressed by one of the genes.
39. The kit of paragraph 36 consisting essentially of antibodies or antigen binding portions thereof that specifically bind respective polypeptides encoded by genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC 11A and PARK7, wherein each antibody or antigen-binding portion thereof specifically binds to a protein expressed by one of the genes.
40. The kit of paragraph 37 consisting essentially of antibodies or antigen binding portions thereof that specifically bind respective polypeptides encoded by genes of SEQ ID NOs 1-141 or a subset thereof, wherein the subset comprises wherein the subset comprises at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7, wherein each antibody or antigen-binding portion thereof specifically binds to a protein expressed by one of the genes.
41. The kit of any one of paragraphs 36-40, wherein the antibodies or antigen binding fragments thereof are immobilized on a solid support.
42. A computer readable medium or computer program product comprising a classifier that predicts the DLBCL-subtype, based on weighted expression of genes of SEQ ID NOs 1-141 or a subset thereof in a sample from a subject having or suspected of having DLBCL, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC11A and PARK7, said classifier having been trained by in silico analysis and classification algorithms.
43. The computer readable medium or computer program product of paragraph 33, wherein the classifier has been trained with training data from a plurality of DLBCL patients, wherein the training data comprise for each of the plurality of DLBCL patients (a) weighted gene expression level of at least the plurality of genes for which the expression levels are measured including said at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7 and (b) information with respect to subtype of DLBCL based on the weighted gene expression level.
44. The computer readable medium or computer program product of any one of paragraphs 42-43, wherein said classifier is trained by one or more algorithms selected from the group consisting of dual ensemble, generalized simulated annealing, T-filter, CORG, CORG combined with support vector machine, dual bagging, single and pairs, forward learning, Laplacian based learning and learning method based on network perturbation amplitude.
45. The computer readable medium or computer program product of any one of paragraphs 42-44, wherein said classifier is trained with at least the data in the Gene Expression Omnibus datasets GSE2109, GSE 10245, GSE1 8842 and GSE37745.
46. A method of treating a subject for Diffuse Large B Cell Lymphoma (DLBCL), the method comprising: (a) assaying a biological sample from a cancer cell of an individual with DLBCL for the expression of at least 15 of the genes corresponding to SEQ ID NOs 1-141; (b) normalizing the expression data for each of the assayed genes to a control; and (c) administering a therapeutic regimen for the treatment of DLBCL of the BCR subtype if the expression of at least four genes of the group consisting of TRMU, CKAP5, PLCG2, FUS, WEE1, ITPR3, SNRPA, PKMYT1, and SUPT5H differs from that of the control; or (d) administering a therapeutic regimen for the treatment of DLBCL of the Host Receptor subtype if the expression of at least two genes of the group consisting of PD-L1, CTLA4, IL15RA, GNS, PTPRM, AMICA, CFH, CD2, ITGAL, ACTN1, A2M and IL2RB differs from that of the control; or (e) administering a therapeutic regimen for the treatment of DLBCL of the OxPhos subtype if the expression of at least three genes of the group consisting of SPCS3, SUCLG1, NDUFAB1, FADD, MRPS16, ATP6V1D, NDUFB1, NDUFB3, SEC 11A and PARK7 differs from that of the control.
47. A method of treating a subject for Diffuse Large B Cell Lymphoma (DLBCL), the method comprising: (a) assaying a biological sample from a cancer cell of an individual with DLBCL for the expression of at least 6 of the genes encoding TRMU, CKAP5, PLCG2, FUS, WEE1, ITPR3, SNRPA, PKMYT1, and SUPT5H; (b) normalizing the expression data for each of the assayed genes to a control; and (c) administering a therapeutic regimen for the treatment of DLBCL of the BCR subtype if the expression of at least four of the genes encoding TRMU, CKAP5, PLCG2, FUS, WEE1, ITPR3, SNRPA, PKMYT1, and SUPT5H differs from that of the control; or (d) administering a therapeutic regimen for the treatment of DLBCL of the Host Receptor or OxPhos subtype if the expression of at least four of the genes encoding TRMU, CKAP5, PLCG2, FUS, WEE1, ITPR3, SNRPA, PKMYT1, and SUPT5H does not differ from that of the control.
48. A method of treating a subject for Diffuse Large B Cell Lymphoma (DLBCL), the method comprising: (a) assaying a biological sample from a cancer cell of an individual with DLBCL for the expression of at least two genes of the group consisting of PD-L1, CTLA4, IL15RA, GNS, PTPRM, AMICA, CFH, CD2, ITGAL, ACTN1, A2M and IL2RB; (b) normalizing the expression data for each of the assayed genes to a control; and (c) administering a therapeutic regimen for the treatment of DLBCL of the Host Receptor subtype if the expression of at least two of the genes encoding PD-L1, CTLA4, IL15RA, GNS, PTPRM, AMICA, CFH, CD2, ITGAL, ACTN1, A2M and IL2RB differs from that of the control; or (d) administering a therapeutic regimen for the treatment of DLBCL of the BCR or OxPhos subtype if the expression of at least two of the genes encoding PD-L1, CTLA4, IL15RA, GNS, PTPRM, AMICA, CFH, CD2, ITGAL, ACTN1, A2M and IL2RB does not differ from that of the control.
49. A method of treating a subject for Diffuse Large B Cell Lymphoma (DLBCL), the method comprising: (a) assaying a biological sample from a cancer cell of an individual with DLBCL for the expression of at least six genes of the group consisting of SPCS3, SUCLG1, NDUFAB1, FADD, MRPS16, ATP6V1D, NDUFB1, NDUFB3, SEC11A and PARK7; (b) normalizing the expression data for each of the assayed genes to a control; and (c) administering a therapeutic regimen for the treatment of DLBCL of the OxPhos subtype if the expression of at least three of the genes encoding SPCS3, SUCLG1, NDUFAB1, FADD, MRPS16, ATP6V1D, NDUFB1, NDUFB3, SEC11A and PARK7 differs from that of the control; or (d) administering a therapeutic regimen for the treatment of DLBCL of the BCR or Host Receptor subtype if the expression of at least three of the genes encoding SPCS3, SUCLG1, NDUFAB1, FADD, MRPS16, ATP6V1D, NDUFB1, NDUFB3, SEC 11A and PARK7 does not differ from that of the control.
50. A method of treating an individual for cancer, the method comprising: (a) assaying a biological sample from a cancer cell of an individual suspected having or having DLBCL for the expression of at least 15 of the genes corresponding to SEQ ID NOs 1-141; (b) normalizing the expression data for each of the assayed genes to a control; (c) administering a therapeutic agent for the treatment of DLBCL when the expression of at least two of the genes corresponding to SEQ ID NOs 1-141 differs from that of the control; and administering a therapeutic agent for the treatment of non-DLBCL cancer when the expression of at least two of the genes corresponding to SEQ ID NOs 1-141 does not differ from the control.
51. The method of paragraph 40, wherein the cancer is DLBCL.
52. The method of any one of paragraphs 36-40, wherein the control is a healthy control.
53. The method of any one of paragraphs 36-41, wherein the subject is human.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology described herein is further illustrated by the following examples which in no way should be construed as being further limiting.

### Example 1

### Methods

### Affymetrix Data

Discovery Set I (Friedberg, J. W. et al., 2008; Monti, S. et al., 2005) consists of 141 DLBCL tumor samples profiled for gene expression on the Affymetrix U133A/B chip pair (see Table 3). This dataset was used to derive the comprehensive consensus clustering (CCC) labels (Monti, S. et al., 2005). A second DLBCL dataset (Monti, S. et al., 2012) consists of 116 DLBCL samples profiled on the Affymetrix U133Plus2.0 chip, with its CCC labels derived based on the ensemble classifier previously described (Polo, J. M. et al., 2007). For 44 of these 116 samples, expression profiles from formalin-fixed material are also available. The 44-sample dataset is referred to as the validation set, and the remaining 72-sample dataset is referred to as Discovery Set II. Two additional datasets profiled on the Affymetrix U133Plus2.0 chip were used for validation: the Lohr dataset, consisting of 57 primary DLBCL samples; and the Lenz dataset (E-GEOD-10846), consisting of 414 primary DLBCL samples. The Lenz dataset consists of two distinct cohorts: a 181-sample cohort corresponding to patients treated with the older CHOP (cyclophosphamide, doxorubicin, vincristine, and prednisone) regimen; and a 233-sample cohort corresponding to patients treated with the current Rituximab-CHOP or R-CHOP regimen. Although all samples were collected pre-treatment, initial exploratory analysis showed a considerable batch effect between the CHOP and R-CHOP portion of the dataset.

### Table 3 Shows Discovery Dataset of 176 Diffuse Large B-Cell Lymphomas Profiled on the Affymetrix U133A/B Platform.

This table contains phenotype information for this set. 141 samples within the set are labeled as meta-consensus and were robustly identified to belong to one of three classe: BCR, OxPhos and HR. For classifier training purposes only these 141 samples were used.

| | Ensemble | Prediction_confidence | COO |
|---|---|---|---|
| DLBCL.NEW.205 | BCR | meta-consensus | ABC |
| DLBCL.NEW.206 | BCR | meta-consensus | GCB |
| DLBCL.NEW.209 | OxPhos | meta-consensus | ABC |
| DLBCL.NEW.210 | OxPhos | meta-consensus | ABC |
| DLBCL.NEW.211 | OxPhos | meta-consensus | GCB |
| DLBCL.NEW.215 | BCR | meta-consensus | ABC |
| DLBCL.NEW.217 | BCR | meta-consensus | NA |
| DLBCL.NEW.219 | OxPhos | meta-consensus | GCB |
| DLBCL.NEW.225 | OxPhos | meta-consensus | GCB |
| DLBCL.NEW.230 | BCR | meta-consensus | ABC |
| DLBCL.NEW.232 | BCR | meta-consensus | GCB |
| DLBCL.NEW.234 | HR | predicted | ABC |
| DLBCL.NEW.239 | HR | meta-consensus | NA |
| DLBCL.NEW.240 | HR | meta-consensus | NA |
| DLBCL.NEW.242 | BCR | meta-consensus | NA |
| DLBCL.NEW.243 | OxPhos | meta-consensus | GCB |
| DLBCL.NEW.244 | OxPhos | meta-consensus | GCB |
| DLBCL.NEW.245 | HR | meta-consensus | NA |
| DLBCL.NEW.246 | HR | meta-consensus | ABC |
| DLBCL.NEW.249 | HR | meta-consensus | NA |
| DLBCL.NEW.250 | BCR | meta-consensus | ABC |
| DLBCL.NEW.251 | BCR | meta-consensus | ABC |
| DLBCL.NEW.254 | HR | meta-consensus | GCB |
| DLBCL.NEW.259 | HR | predicted | NA |
| DLBCL.NEW.261 | BCR | meta-consensus | GCB |
| DLBCL.NEW.262 | OxPhos | meta-consensus | ABC |
| DLBCL.NEW.267 | BCR | meta-consensus | GCB |
| DLBCL.NEW.268 | BCR | meta-consensus | GCB |
| DLBCL.NEW.269 | BCR | meta-consensus | GCB |
| DLBCL.NEW.270 | BCR | meta-consensus | NA |
| DLBCL.NEW.271 | OxPhos | meta-consensus | ABC |
| DLBCL.NEW.272 | OxPhos | predicted | ABC |
| DLBCL.NEW.274 | BCR | predicted | NA |
| DLBCL.NEW.277 | BCR | meta-consensus | GCB |
| DLBCL.NEW.278 | BCR | predicted | GCB |
| DLBCL.NEW.279 | HR | predicted | ABC |
| DLBCL.NEW.280 | HR | meta-consensus | GCB |
| DLBCL.NEW.282 | HR | meta-consensus | ABC |
| DLBCL.NEW.283 | BCR | meta-consensus | ABC |
| DLBCL.NEW.284 | BCR | meta-consensus | ABC |
| DLBCL.NEW.285 | BCR | meta-consensus | ABC |
| DLBCL.NEW.286 | BCR | meta-consensus | GCB |
| DLBCL.NEW.287 | OxPhos | predicted | GCB |
| DLBCL.NEW.288 | OxPhos | meta-consensus | GCB |
| DLBCL.NEW.289 | OxPhos | meta-consensus | ABC |
| DLBCL.NEW.290 | OxPhos | meta-consensus | GCB |
| DLBCL.NEW.291 | BCR | meta-consensus | NA |
| DLBCL.NEW.292 | HR | meta-consensus | ABC |
| DLBCL.NEW.293 | OxPhos | meta-consensus | NA |
| DLBCL.NEW.295 | HR | meta-consensus | ABC |
| DLBCL.NEW.299 | HR | predicted | GCB |
| DLBCL.NEW.300 | OxPhos | predicted | ABC |
| DLBCL.NEW.301 | BCR | meta-consensus | NA |
| DLBCL.NEW.303 | HR | meta-consensus | NA |
| DLBCL.NEW.304 | BCR | meta-consensus | GCB |
| DLBCL.NEW.305 | OxPhos | meta-consensus | NA |
| DLBCL.NEW.306 | BCR | predicted | NA |
| DLBCL.NEW.307 | HR | meta-consensus | GCB |
| DLBCL.NEW.309 | OxPhos | predicted | GCB |
| DLBCL.NEW.310 | OxPhos | meta-consensus | GCB |
| DLBCL.NEW.311 | OxPhos | meta-consensus | ABC |
| DLBCL.NEW.312 | OxPhos | meta-consensus | GCB |
| DLBCL.NEW.313 | BCR | meta-consensus | GCB |
| DLBCL.NEW.332 | HR | predicted | NA |
| DLBCL.NEW.333 | BCR | meta-consensus | GCB |
| DLBCL.NEW.336 | HR | meta-consensus | NA |
| DLBCL.NEW.338 | HR | meta-consensus | NA |
| DLBCL.NEW.339 | BCR | meta-consensus | ABC |
| DLBCL.NEW.340 | BCR | meta-consensus | ABC |
| DLBCL.NEW.344 | BCR | meta-consensus | GCB |
| DLBCL.NEW.345 | BCR | meta-consensus | GCB |
| DLBCL.NEW.346 | HR | meta-consensus | NA |
| DLBCL.NEW.347 | BCR | meta-consensus | GCB |
| DLBCL.NEW.348 | HR | meta-consensus | NA |
| DLBCL.NEW.349 | HR | meta-consensus | NA |
| DLBCL.NEW.350 | BCR | predicted | ABC |
| DLBCL.NEW.352 | OxPhos | predicted | GCB |
| DLBCL.NEW.353 | HR | meta-consensus | NA |
| DLBCL.NEW.357 | BCR | meta-consensus | ABC |
| DLBCL.NEW.359 | BCR | meta-consensus | ABC |
| DLBCL.NEW.361 | HR | meta-consensus | GCB |
| DLBCL.NEW.401 | HR | meta-consensus | NA |
| DLBCL.NEW.402 | HR | meta-consensus | NA |
| DLBCL.NEW.404 | BCR | predicted | GCB |
| DLBCL.NEW.405 | OxPhos | meta-consensus | ABC |
| DLBCL.NEW.407 | OxPhos | meta-consensus | GCB |
| DLBCL.NEW.408 | OxPhos | meta-consensus | NA |
| DLBCL.NEW.410 | BCR | predicted | ABC |
| DLBCL.NEW.411 | OxPhos | meta-consensus | GCB |
| DLBCL.NEW.412 | OxPhos | meta-consensus | GCB |
| DLBCL.NEW.413 | HR | meta-consensus | NA |
| DLBCL.NEW.414 | BCR | meta-consensus | GCB |
| DLBCL.NEW.416 | HR | meta-consensus | ABC |
| DLBCL.NEW.417 | OxPhos | meta-consensus | GCB |
| DLBCL.NEW.418 | OxPhos | meta-consensus | ABC |
| DLBCL.NEW.419 | BCR | meta-consensus | ABC |
| DLBCL.NEW.421 | BCR | predicted | GCB |
| DLBCL.NEW.422 | BCR | predicted | GCB |
| DLBCL.NEW.423 | BCR | meta-consensus | NA |
| DLBCL.NEW.424 | HR | meta-consensus | GCB |
| DLBCL.NEW.425 | HR | predicted | GCB |
| DLBCL.NEW.426 | OxPhos | meta-consensus | GCB |
| DLBCL.NEW.427 | HR | predicted | ABC |
| DLBCL.NEW.428 | HR | meta-consensus | NA |
| DLBCL.NEW.429 | OxPhos | meta-consensus | GCB |
| DLBCL.NEW.430 | HR | meta-consensus | NA |
| DLBCL.NEW.432 | BCR | meta-consensus | GCB |
| DLBCL.NEW.433 | BCR | meta-consensus | GCB |
| DLBCL.NEW.434 | HR | meta-consensus | NA |
| DLBCL.NEW.435 | HR | meta-consensus | ABC |
| DLBCL.NEW.436 | BCR | predicted | GCB |
| DLBCL.NEW.437 | BCR | meta-consensus | NA |
| DLBCL.NEW.438 | HR | meta-consensus | ABC |
| DLBCL.NEW.441 | HR | predicted | NA |
| DLBCL.NEW.442 | BCR | meta-consensus | ABC |
| DLBCL.NEW.443 | OxPhos | meta-consensus | ABC |
| DLBCL.NEW.445 | BCR | meta-consensus | GCB |
| DLBCL.NEW.446 | HR | meta-consensus | NA |
| DLBCL.NEW.447 | BCR | meta-consensus | NA |
| DLBCL.NEW.448 | BCR | meta-consensus | GCB |
| DLBCL.NEW.449 | HR | meta-consensus | GCB |
| DLBCL.NEW.450 | BCR | meta-consensus | GCB |
| DLBCL.NEW.451 | HR | predicted | GCB |
| DLBCL.NEW.452 | OxPhos | predicted | GCB |
| DLBCL.NEW.453 | OxPhos | meta-consensus | NA |
| DLBCL.NEW.454 | HR | predicted | GCB |
| DLBCL.NEW.455 | BCR | meta-consensus | ABC |
| DLBCL.NEW.456 | HR | meta-consensus | NA |
| DLBCL.NEW.458 | HR | meta-consensus | GCB |
| DLBCL.NEW.460 | BCR | meta-consensus | ABC |
| DLBCL.NEW.461 | BCR | predicted | GCB |
| DLBCL.NEW.462 | BCR | meta-consensus | GCB |
| DLBCL.NEW.463 | BCR | meta-consensus | GCB |
| DLBCL.NEW.464 | OxPhos | meta-consensus | ABC |
| DLBCL.NEW.465 | BCR | meta-consensus | GCB |
| DLBCL.NEW.466 | OxPhos | meta-consensus | ABC |
| DLBCL.NEW.467 | BCR | meta-consensus | GCB |
| DLBCL.NEW.468 | HR | meta-consensus | NA |
| DLBCL.NEW.469 | OxPhos | predicted | GCB |
| DLBCL.NEW.470 | OxPhos | meta-consensus | ABC |
| DLBCL.NEW.471 | OxPhos | meta-consensus | ABC |
| DLBCL.NEW.472 | OxPhos | meta-consensus | NA |
| DLBCL.NEW.473 | OxPhos | meta-consensus | GCB |
| DLBCL.NEW.474 | BCR | meta-consensus | NA |
| DLBCL.NEW.475 | BCR | predicted | ABC |
| DLBCL.NEW.476 | HR | meta-consensus | NA |
| DLBCL.NEW.477 | BCR | predicted | NA |
| DLBCL.NEW.478 | HR | meta-consensus | ABC |
| DLBCL.NEW.479 | HR | meta-consensus | NA |
| DLBCL.NEW.481 | BCR | predicted | GCB |
| DLBCL.NEW.482 | HR | meta-consensus | NA |
| DLBCL.NEW.483 | HR | meta-consensus | ABC |
| DLBCL.NEW.484 | OxPhos | meta-consensus | NA |
| DLBCL.NEW.485 | HR | predicted | NA |
| DLBCL.NEW.486 | OxPhos | meta-consensus | ABC |
| DLBCL.NEW.489 | HR | predicted | NA |
| DLBCL.NEW.490 | HR | meta-consensus | NA |
| DLBCL.NEW.491 | OxPhos | meta-consensus | NA |
| DLBCL.NEW.492 | OxPhos | meta-consensus | ABC |
| DLBCL.NEW.494 | OxPhos | meta-consensus | NA |
| DLBCL.NEW.495 | OxPhos | meta-consensus | GCB |
| DLBCL.NEW.496 | OxPhos | meta-consensus | ABC |
| DLBCL.NEW.497 | OxPhos | meta-consensus | GCB |
| DLBCL.NEW.498 | OxPhos | meta-consensus | GCB |
| DLBCL.NEW.501 | OxPhos | meta-consensus | ABC |
| DLBCL.NEW.502 | OxPhos | meta-consensus | NA |
| DLBCL.NEW.503 | BCR | predicted | GCB |
| DLBCL.NEW.504 | OxPhos | meta-consensus | GCB |
| DLBCL.NEW.506 | BCR | meta-consensus | GCB |
| DLBCL.NEW.507 | OxPhos | meta-consensus | NA |
| DLBCL.NEW.509 | HR | predicted | NA |
| DLBCL.NEW.512 | OxPhos | meta-consensus | GCB |
| DLBCL.NEW.513 | HR | meta-consensus | NA |
| DLBCL.NEW.514 | HR | meta-consensus | NA |
| DLBCL.NEW.609 | HR | predicted | NA |
| DLBCL.NEW.617 | OxPhos | meta-consensus | NA |

### Selection of Markers

Linear models were used for microarrays (Smyth, G. Limma, 2005) as implemented in the R/Bioconductor package limma to identify differentially expressed genes, and gene set enrichment analysis (GSEA) (Subramanian, A. et al., 2005) to look for differentially regulated pathways.

### Nanostring Profiling on Validation Cohort

The Nanostring platform relies on housekeeping genes for cross-sample normalization, which were selected based on the following criteria evaluated in Discovery Set I: i) minimum variance across samples; ii) even coverage of the range of measured gene expression in the data, by partitioning the expression range into eight tiers, from 4 to 12 (in log 2 space), and by selecting two genes from each tier; and iii) lack of differential expression with respect to the CCC classifications. The resulting 16 genes are listed in Table 4 below.

Table 4 Shows Housekeeping Genes. The Table Contains the Differential Expression Statistics Between all CCC and COO Subtypes in the Discovery Set I.

| | | Consensus Clustering Classification (CCC) | | | | | | | |
|---|---|---|---|---|---|---|---|---|---|
| gene | Variable | | | asymp. | | | | | |
| symbol | Importance | class | asymp.p | fdr | fold.chg | median.0 | median.1 | mad.0 | mad.1 |
| GNAL | HOUSE- | HR | 0.88528 | 0.9129 | 1.0019 | 13.722 | 13.6959 | 2.1281 | 2.5119 |
| | KEEPING 3-4 | | | | | | | | |
| BHLHE22 | HOUSE- | BCR | 0.898761 | 0.9421 | 1.0031 | 15.5957 | 15.5482 | 1.8669 | 1.8705 |
| | KEEPING 3-4 | | | | | | | | |
| BHMT2 | HOUSE- | OxPhos | 0.874394 | 0.9208 | 1.0099 | 18.9533 | 19.1412 | 2.4053 | 2.335 |
| | KEEPING 4-5 | | | | | | | | |
| EPHA4 | HOUSE- | BCR | 0.875591 | 0.9285 | 1.0632 | 20.2851 | 19.0789 | 5.9186 | 5.8684 |
| | KEEPING 4-5 | | | | | | | | |
| EPHB2 | HOUSE- | HR | 0.822766 | 0.8624 | 1.0115 | 37.476 | 37.9076 | 4.4956 | 5.2847 |
| | KEEPING 5-6 | | | | | | | | |
| SERPINA3 | HOUSE- | OxPhos | 0.89041 | 0.9318 | 1.0371 | 34.683 | 35.9709 | 18.0233 | 17.978 |
| | KEEPING 5-6 | | | | | | | | |
| TRPC4AP | HOUSE- | BCR | 0.905261 | 0.946 | 1.0517 | 111.0755 | 116.8228 | 17.1212 | 15.4305 |
| | KEEPING 6-7 | | | | | | | | |
| HAMP | HOUSE- | OxPhos | 0.809777 | 0.8779 | 1.0582 | 97.7369 | 103.4245 | 67.1461 | 83.7154 |
| | KEEPING 6-7 | | | | | | | | |
| SECISBP2 | HOUSE- | BCR | 0.911654 | 0.9495 | 1.0472 | 241.9692 | 253.3958 | 36.8706 | 33.3422 |
| | KEEPING 7-8 | | | | | | | | |
| MEIS2 | HOUSE- | OxPhos | 0.80169 | 0.8723 | 1.0533 | 127.1017 | 133.8825 | 77.4465 | 78.0664 |
| | KEEPING 7-8 | | | | | | | | |
| KXD1 | HOUSE- | BCR | 0.753227 | 0.8521 | 1.0593 | 393.0849 | 416.3985 | 68.1711 | 63.1752 |
| | KEEPING 8-9 | | | | | | | | |
| PSMC5 | HOUSE- | BCR | 0.560477 | 0.7155 | 1.1459 | 752.5609 | 862.374 | 90.4758 | 142.9001 |
| | KEEPING 9-10 | | | | | | | | |
| SARS | HOUSE- | BCR | 0.608208 | 0.7531 | 1.0979 | 557.7806 | 612.4092 | 92.5116 | 103.7974 |
| | KEEPING 9-10 | | | | | | | | |
| EMC4 | HOUSE- | OxPhos | 0.826579 | 0.8897 | 1.069 | 963.9959 | 1030.512 | 162.9206 | 250.7661 |
| | KEEPING | | | | | | | | |
| | 10-11 | | | | | | | | |
| KPNB1 | HOUSE- | BCR | 0.52631 | 0.6878 | 1.1089 | 1653.199 | 1833.287 | 226.7277 | 247.6397 |
| | KEEPING | | | | | | | | |
| | 10-11 | | | | | | | | |
| CYBA | HOUSE- | OxPhos | 0.3147 | 0.4594 | 1.0392 | 2343.503 | 2435.444 | 472.4336 | 735.1535 |
| | KEEPING | | | | | | | | |
| | 11-12 | | | | | | | | |
| | | Cell-0f-origin (COO) | | | | | | | |
| gene | Variable | COO | | | | | | | |
| symbol | Importance | class | pval | fdr | asymp.p.1 | asymp.fdr.1 | fold.chg.1 | median.ABC | median.GCB |
| GNAL | HOUSE- | ABC | 0.513487 | 0.76882 | 0.486335 | 0.741195 | 1.045 | 13.9284 | 13.3286 |
| | KEEPING 3-4 | | | | | | | | |
| BHLHE22 | HOUSE- | ABC | 0.619381 | 0.833103 | 0.635616 | 0.832857 | 1.0051 | 15.5521 | 15.6317 |
| | KEEPING 3-4 | | | | | | | | |
| BHMT2 | HOUSE- | ABC | 0.633367 | 0.84013 | 0.629583 | 0.829773 | 1.0548 | 19.1412 | 18.1471 |
| | KEEPING 4-5 | | | | | | | | |
| EPHA4 | HOUSE- | GCB | 0.913087 | 0.968084 | 0.949302 | 0.982289 | 1.0725 | 20.9444 | 19.5286 |
| | KEEPING 4-5 | | | | | | | | |
| EPHB2 | HOUSE- | GCB | 0.527473 | 0.777678 | 0.533491 | 0.77195 | 1.0327 | 37.4812 | 38.7067 |
| | KEEPING 5-6 | | | | | | | | |
| SERPINA3 | HOUSE- | ABC | 0.677323 | 0.862692 | 0.720759 | 0.875975 | 1.0101 | 34.219 | 34.5651 |
| | KEEPING 5-6 | | | | | | | | |
| TRPC4AP | HOUSE- | ABC | 0.789211 | 0.918956 | 0.794975 | 0.91335 | 1.0329 | 115.5474 | 111.8683 |
| | KEEPING 6-7 | | | | | | | | |
| HAMP | HOUSE- | GCB | 0.865135 | 0.951284 | 0.863866 | 0.945096 | 1.1809 | 84.1936 | 99.4202 |
| | KEEPING 6-7 | | | | | | | | |
| SECISBP2 | HOUSE- | GCB | 0.727273 | 0.886488 | 0.717111 | 0.874192 | 1.0443 | 251.9257 | 241.2433 |
| | KEEPING 7-8 | | | | | | | | |
| MEIS2 | HOUSE- | GCB | 0.911089 | 0.967471 | 0.906698 | 0.963452 | 1.0012 | 125.8898 | 126.0377 |
| | KEEPING 7-8 | | | | | | | | |
| KXD1 | HOUSE- | GCB | 0.30969 | 0.611655 | 0.284388 | 0.577205 | 1.0269 | 391.1836 | 401.6885 |
| | KEEPING 8-9 | | | | | | | | |
| PSMC5 | HOUSE- | ABC | 0.123876 | 0.377887 | 0.112177 | 0.359016 | 1.1071 | 816.2706 | 737.2749 |
| | KEEPING 9-10 | | | | | | | | |
| SARS | HOUSE- | GCB | 0.377622 | 0.671959 | 0.352396 | 0.641504 | 1.0359 | 563.3866 | 583.5984 |
| | KEEPING 9-10 | | | | | | | | |
| EMC4 | HOUSE- | GCB | 0.817183 | 0.929851 | 0.77882 | 0.905022 | 1.0053 | 985.0672 | 979.8933 |
| | KEEPING | | | | | | | | |
| | 10-11 | | | | | | | | |
| KPNB1 | HOUSE- | GCB | 0.871129 | 0.952605 | 0.789965 | 0.910981 | 1.0102 | 1721.594 | 1704.217 |
| | KEEPING | | | | | | | | |
| | 10-11 | | | | | | | | |
| CYBA | HOUSE- | GCB | 0.5475 | 0.7898 | 0.5465 | 0.7798 | 1.0311 | 2321.999 | 2394.163 |
| | KEEPING | | | | | | | | |
| | 11-12 | | | | | | | | |

### Tumor and Patient Cohorts

44 samples were selected from the DLBCL dataset in reference Monti et al. 2012 for which at least 10 out of 13 models in the ensemble classifier (Polo, et al. 2007) resulted in the same CCC classification. This validation cohort had both frozen and paired formalin-fixed, paraffin-embedded (FFPE) tissue available. The validation set consisted of 14 BCR, 16 HR and 14 OxPhos samples. Of note, the 44 samples were excluded from the feature selection process to ensure unbiased classification performance testing. RNA extraction from frozen tissue was performed as previously described (Monti et al. 2012). For RNA extraction from FFPE tissue standard protocols were followed using the Qiagen FFPE-RNA extraction kit. The Nanostring assay was performed in the Dana-Faber Cancer Institute Microarray core following standard protocols. Briefly, RNAs were assessed for quality and concentration using Agilent Bioanalyzer RNA Nano or Pico chips and a smear analysis was performed using Agilent 2100 Expert software to quantify the percentage of RNA fragments greater than 300 nt in each sample. Thereafter, 100 ng of RNA with a fragment size of greater than 300 nt was profiled using the custom probe set on Nanostring (Geiss et al. 2008). The custom probe set was composed of 275 probes ordered directly from Nanostring (38 BCR, 55 OxPhos, 38 HR and 16 housekeeping genes). Capture and Reporter Code sets were added to the samples following manufacturer's protocol and allowed to hybridize at 65° C. for 16 hrs. Samples were washed and loaded onto a cartridge using the nCounter Analysis System Prep Station per manufacturer's recommendations. The cartridge was scanned using the nCounter Digital Analyzer at the maximum resolution of 1150 FOV.

### Data Preprocessing

All Affymetrix microarray data were normalized based on the Robust Multi-Array Average (RMA) procedure (Irizarry, R. A., 2003) implemented in the R/Bioconductor package affy. Probes' annotation by Ensembl gene identifiers was based on custom Brainarray CDFs version 18 (Dai, M. et al., 2005). The Nanostring data was normalized using the R package NanoStringNorm (Waggott, D. et al., 2012). Mapping from Ensembl gene identifiers to Gene Symbols was performed using the R/Bioconductor package biomaRt (Kasprzyk, A., 2011).

To minimize potential batch effects among different datasets, gene-specific normalization was performed, whereby the expression level y, of gene i in sample j in the test dataset is transformed as follows:

$${y}_{ij} = \frac{{x}_{j} - {\overline{y}}_{i}}{{σ}_{yi}} {σ}_{xi} + {\overline{x}}_{ɛ}$$

where x̅ᵢ and y̅ᵢ are gene i's means within the training and test dataset, respectively, and σₓᵢ and σ_{yi} are the corresponding standard deviations. This transformation is based on the assumption that samples in both datasets are drawn from the same population and corrects for systematic measuring biases.

### Classification Models

Most prediction models were inferred based on the Elastic net algorithm (Hui Zou, T. H. Regularization and variable selection via the Elastic Net) as implemented in the R package glmnet. Elastic net was selected because of its superior predictive performance within the Discovery Sets, as well as because of its interpretability, since the resulting classifier outputs gene-specific coefficients that can be directly mapped to the genes' importance in driving the classification. For comparison purposes, we also tested Random forest (Breiman, L. Random Forests, 2001) and Shrunken Centroid (Hastie, T. et al., 2011) classifiers as implemented in the R packages randomForest and pamr, respectively. For the assessment of each classifier's prediction performance, accuracy and within-class sensitivity/specificity for all three subtypes were measured. For the assessment of the prediction performance within a dataset, 10-fold cross-validation (10-CV) in the larger Affymetrix datasets, and leave-one-out cross-validation (LOO-CV) in the Nanostring datasets were used.

### Example 2

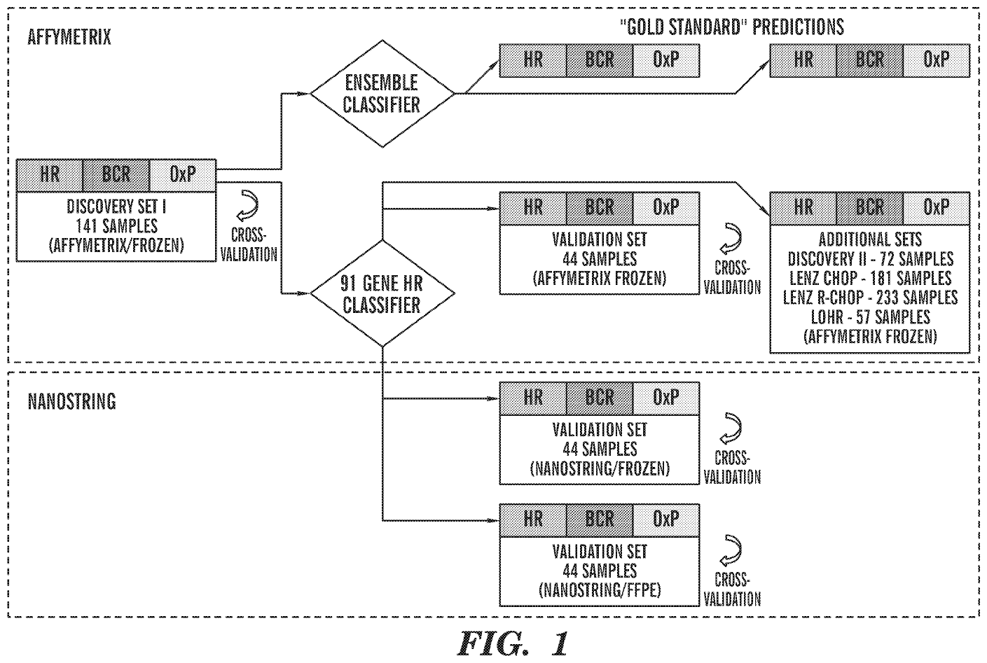

An overview on the experimental design is presented in FIG. 1. First, a parsimonious classifier based on a carefully selected set of CCC genes (CCC signature) was inferred. To this end, cross-validation was used within Discovery Set I to compare competing classification methods; the best performing classifier was then applied to multiple publicly available Affymetrix DLBCL datasets and compared its predictions to those of the original ensemble classifier (Polo, J. M. et al., 2007). The parsimonious classifier was then validated on the fresh frozen validation dataset, and finally on the FFPE validation dataset, both profiled on the Nanostring platform.

### CCC Signature Selection

An initial set of candidate genes was identified based on their significant enrichment in KEGG, Biocarta and Reactome pathways as tested by GSEA with respect to the CCC phenotype. The union of the leading edge genes of the top 20 gene sets in each class was used. This initial list was filtered based on signal robustness and significance within Discovery Sets I and II, by selecting only genes with fold-change higher than 2.5, false discovery rate (FDR) less than 0.05, and average microarray intensity value greater than 64 (26). The significance of the differential expression was assessed by moderated t-test as implemented in limma (Smyth, G. Limma, 2005). This procedure yielded a list of genes, partitioned into n1, n2, and n3 markers of the OxPhos, BCR, and HR classes, respectively. Finally, an Elastic Net model (Hui Zou, T. H. Regularization and variable selection via the Elastic Net) was built from Discovery Set I, and used the estimated genes' coefficients to further prune the candidate list, since the Elastic net-based estimation shrinks to zero the weights of those genes that do not contribute to the classification. The final signature consists of 141 genes (Table 1 and Table 2), which was used with all classification models in all subsequent evaluations.

### Selection of Classification Model

10-fold cross-validation was run on both discovery sets and compared three classifiers: Elastic Net, Random Forest and Shrunken Centroids. The results are summarized in Table 5. In both datasets, Elastic Net outperformed the Random Forest model, and in one set the Shrunken Centroids (with accuracies of 96.5 vs. 92.2 and 97.1% in Discovery Set I, and 91.8 vs. 91.8% and 90.4% in Discovery Set II). Based on these results, Elastic Net was the classifier of choice, to be evaluated on the validation datasets.

Table 5 Shows Comparison Between Different Classification Methods.

In this table, leastic net classification model is compared to two other state-of-the-art prediction models: Random Forest and Shrunken Centroids (PAM). The three were compared in both the discovery sets. The leastic net outperforms the Random Forest in both datasets and the Shrunken Centroids in the second dataset. (ACC: accuracy, SENS: sensitivity, SPEC: specificity)

| | Elastic Net | Random Forest | Shrunken Centroid |
|---|---|---|---|
| | Discovery I - 10 fold CV | | |
| ACC | 0.965 | 0.922 | 0.971 |
| SENS - BCR | 0.980 | 0.940 | 0.98 |
| SPEC - BCR | 0.956 | 0.945 | 0.978 |
| SENS - HR | 0.905 | 0.810 | 0.929 |
| SPEC - HR | 1.000 | 0.980 | 1.000 |
| SENS - OxP | 1.000 | 1.000 | 1.000 |
| SPEC - OxP | 0.989 | 0.957 | 0.978 |
| | Discovery II - 10 fold CV | | |
| ACC | 0.918 | 0.918 | 0.904 |
| SENS - BCR | 0.840 | 0.960 | 0.840 |
| SPEC - BCR | 0.979 | 0.958 | 1.000 |
| SENS - HR | 1.000 | 0.864 | 1.000 |
| SPEC - HR | 0.907 | 0.941 | 0.882 |
| SENS - OxP | 0.889 | 0.923 | 0.885 |
| SPEC - OxP | 0.982 | 0.979 | 0.979 |

The optimal parameters for classification were selected by maximization of accuracy as estimated by 10-fold cross-validation. Both the parameter alpha, which determines the trade-off between LASSO (Tibshirani, R. Regression Shrinkage and Selection Via the Lasso) and Tikhonov regularization, and the shrinkage parameter lambda were set to 0.1. A final parsimonious Elastic Net model was then trained based on the entire Discovery Set I. The weights of the signature genes for all three classes are listed in Table 1.

### Composition of the CCC Signature

The leastic net weights provide a data-driven measure of each gene's importance in distinguishing between the three different subtypes. In this section we will describe the most relevant of these genes. The genes that are up-regulated in the BCR subtype include TRMU, CKAP5, PLCG2, FUS, WEE1, ITPR3, SNRPA, PKMYT1, SUPT5H, the genes in that have a high weight in the host response subtype include PD-L1, CTLA4, IL15RA, GNS, PTPRM, AMICA, CFH, CD2, ITGAL, ACTN1, A2M and IL2RB, most of which are related to the adaptive T cell response of the immune system. And finally the genes that have a high weight in the OxPhos subtype include: SPCS3, SUCLG1, NDUFAB1, FADD, MRPS16, ATP6V1D, NDUFB1, NDUFB3, SEC11A, PARK7, many of which are associated with oxidative phosphorylation or the electron transportation.

To confirm the biological profile of each class' signature, their composition was annotated using a hypergeometric test with respect to the genesets in the C2: canonical pathways category of the Molecular Signature Database (Liberzon et al. 2011). The top gene sets for each signature and corresponding q-values are provided in Table 6. The BCR signature is enriched in gene sets related to proliferation (mRNA processing, mitosis, cell cycle) and B-cell signaling. The HR signature is enriched in gene sets related to complement cascade and other immune related pathways, while the OxPhos signature is enriched in oxidative phosphorylation and other metabolic pathways. Table 6 shows hypergeometric enrichment between CCC signatures and canonical pathways.

| Enriched sets in BCR signature | BCR.fdr |
|---|---|
| REACTOME_MRNA_SPLICING | 5.449E−20 |
| REACTOME_MRNA_PROCESSING | 4.43114E−19 |
| REACTOME_PROCESSING_OF_CAPPED_INTRON_CONTAINING_PRE_MRNA | 2.70705E−18 |
| REACTOME_MITOTIC_G2_G2_M_PHASES | 6.12701E−09 |
| KEGG_SPLICEOSOME | 4.01416E−07 |
| REACTOME_LOSS_OF_NLP_FROM_MITOTIC_CENTROSOMES | 5.34543E−05 |
| REACTOME_RECRUITMENT_OF_MITOTIC_CENTROSOME_PROTEINS_AND_COMPLEXES | 0.000105887 |
| REACTOME_CELL_CYCLE_MITOTIC | 0.000108918 |
| SIG_BCR_SIGNALING_PATHWAY | 0.000569348 |
| REACTOME_CELL_CYCLE | 0.00120007 |
| REACTOME_ANTIGEN_ACTIVATES_B_CELL_RECEPTOR_LEADING_TO_GENERA- | 0.003382154 |
| TION_OF_ SECOND_MESSENGERS | |
| REACTOME_MRNA_3_END_PROCESSING | 0.007342787 |
| ST_B_CELL_ANTIGEN_RECEPTOR | 0.012652131 |
| REACTOME_CLEAVAGE_OF_GROWING_TRANSCRIPT_IN_THE_TERMINATION_REGION_ | 0.018597961 |
| REACTOME_CYCLIN_A_B1_ASSOCIATED_EVENTS_DURING_G2_M_TRANSITION | 0.021334622 |
| REACTOME_RNA_POL_II_TRANSCRIPTION | 0.034527049 |
| PID_BCR_5PATHWAY | 0.087949648 |
| SIG_PIP3_SIGNALING_IN_B_LYMPHOCYTES | 0.317979434 |
| REACTOME_MRNA_SPLICING_MINOR_PATHWAY | 0.618301266 |
| PID_PLK1_PATHWAY | 0.659389286 |
| Enriched sets in HR signature | HR.fdr |
| KEGG_COMPLEMENT_AND_COAGULATION_CASCADES | 1.48E−15 |
| BIOCARTA_COMP_PATHWAY | 1.26E−09 |
| REACTOME_IMMUNE_SYSTEM | 7.34E−09 |
| BIOCARTA_CLASSIC_PATHWAY | 1.98E−08 |
| REACTOME_INITIAL_TRIGGERING_OF_COMPLEMENT | 5.25E−08 |
| REACTOME_HEMOSTASIS | 6.69E−08 |
| REACTOME_COMPLEMENT_CASCADE | 8.01E−08 |
| KEGG_LYSOSOME | 1.09E−07 |
| REACTOME_RESPONSE_TO_ELEVATED_PLATELET_CYTOSOLIC_CA2_ | 5.21E−06 |
| REACTOME_CELL_SURFACE_INTERACTIONS_AT_THE_VASCULAR_WALL | 6.22E−06 |
| KEGG_CELL_ADHESION_MOLECULES_CAMS | 6.78E−06 |
| REACTOME_PLATELET_ACTIVATION_SIGNALING_AND_AGGREGATION | 2.19E−05 |
| REACTOME_CREATION_OF_C4_AND_C2_ACTIVATORS | 8.86E−05 |
| KEGG_SYSTEMIC_LUPUS_ERYTHEMATOSUS | 0.000186 |
| REACTOME_INNATE_IMMUNE_SYSTEM | 0.000349 |
| BIOCARTA_TCYTOTOXIC_PATHWAY | 0.000416 |
| REACTOME_ADAPTIVE_IMMUNE_SYSTEM | 0.003083 |
| NABA_ECM_REGULATORS | 0.010237 |
| REACTOME_IMMUNOREGULATORY_INTERACTIONS_BETWEEN_A_LYMPHOID_ | 0.016791 |
| AND_A_NON_ LYMPHOID_CELL | |
| KEGG_LEISHMANIA_INFECTION | 0.019271 |
| Enriched sets in OxPhos signature | OxPhos.fdr |
| KEGG_PARKINSONS_DISEASE | 1.07E−32 |
| KEGG_OXIDATIVE_PHOSPHORYLATION | 1.67E−32 |
| KEGG_ALZHEIMERS_DISEASE | 1.16E−29 |
| REACTOME_RESPIRATORY_ELECTRON_TRANSPORT_ATP_SYNTHESIS_BY_ | 8.71E−29 |
| CHEMIOSMOTIC_COUPLING_AND_HEAT_PRODUCTION_BY_UNCOUPLING_PROTEINS_ | |
| KEGG_HUNTINGTONS_DISEASE | 1.55E−28 |
| REACTOME_TCA_CYCLE_AND_RESPIRATORY_ELECTRON_TRANSPORT | 2.53E−28 |
| REACTOME_RESPIRATORY_ELECTRON_TRANSPORT | 2.72E−25 |
| KEGG_CARDIAC_MUSCLE_CONTRACTION | 1.14E−06 |
| REACTOME_MITOCHONDRIAL_TRNA_AMINOACYLATION | 0.008379 |
| REACTOME_INFLUENZA_VIRAL_RNA_TRANSCRIPTION_AND_REPLICATION | 0.080488 |
| REACTOME_SRP_DEPENDENT_COTRANSLATIONAL_PROTEIN_TARGETING_ | 0.115645 |
| TO_MEMBRANE | |
| REACTOME_FORMATION_OF_ATP_BY_CHEMIOSMOTIC_COUPLING | 0.140842 |
| REACTOME_TRNA_AMINOACYLATION | 0.142271 |
| KEGG_RIBOSOME | 0.224885 |
| REACTOME_INFLUENZA_LIFE_CYCLE | 0.252441 |
| REACTOME_TRANSLATION | 0.436021 |
| KEGG_AMINOACYL_TRNA_BIOSYNTHESIS | 1 |
| REACTOME_PEPTIDE_CHAIN_ELONGATION | 1 |
| NABA_MATRISOME | 1 |
| REACTOME_SYNTHESIS_SECRETION_AND_INACTIVATION_OF_GIP | 1 |

### CCC Prediction Model in Affymetrix

The parsimonious Elastic net model based on the HR signature was applied on each of the available Affymetrix datasets, and compared its predictions to those of the Ensemble classifier (Polo, J. M. et al., 2007). The results, shown in Table 7, indicate an accuracy ranging from 81.8 to 93.2%. For comparison, the 10-fold cross-validation on both discovery sets and the validation set (Affymetrix) yielded accuracies between 96.9% and 99.7%.

Table 7 Shows Prediction Results Across all Datasets.

All predictions in this table are derived by building an leastic net model on the Discovery Set I, which was then used to predict the class labels across all sets after using gene specific normalization to reduce the batch effect. (ACC: accuracy, SENS: sensitivity, SPEC: specificity)

| | | | | | | Validation | Validation |
|---|---|---|---|---|---|---|---|
| | | Lenz | Lenz | Lenz | Validation | Nanostring | Nanostring |
| | Discovery II | CHOP | R-CHOP | Lohr | Affymetrix | frozen | FFPE |
| Technology | Affymetrix | | | | | Nanostring | |
| Samples | 72 | 181 | 233 | 57 | 44 | 44 | 44 |
| ACC | 0.932 | 0.818 | 0.905579 | 0.859649 | 0.931 | 0.886364 | 0.590909 |
| SENS - BCR | 0.920 | 0.873 | 0.931818 | 0.909091 | 0.949 | 0.928571 | 0.928571 |
| SPEC - BCR | 0.979 | 0.833 | 0.917241 | 0.885714 | 0.961 | 0.966667 | 0.6 |
| SENS - HR | 0.955 | 0.847 | 0.897059 | 0.789474 | 0.919 | 0.9375 | 0.6875 |
| SPEC - HR | 0.941 | 0.963 | 0.951515 | 1 | 0.962 | 0.892857 | 0.892857 |
| SENS - OxP | 0.923 | 0.600 | 0.883117 | 0.875 | 0.925 | 0.785714 | 0.142857 |
| SPEC - OxP | 0.979 | 0.921 | 0.987179 | 0.902439 | 0.974 | 0.966667 | 0.9 |

### CCC Prediction in Nanostring

With the parsimonious classifier established in the Affymetrix Discovery set, its performance was next tested on Nanostring. Thanks to the availability of paired samples profiled on Affymetrix for each of the patients in the Nanostring validation set, whole-transcriptome CCC predictions based on the ensemble classifier (Polo, J. M. et al., 2007) were used as the gold standard.

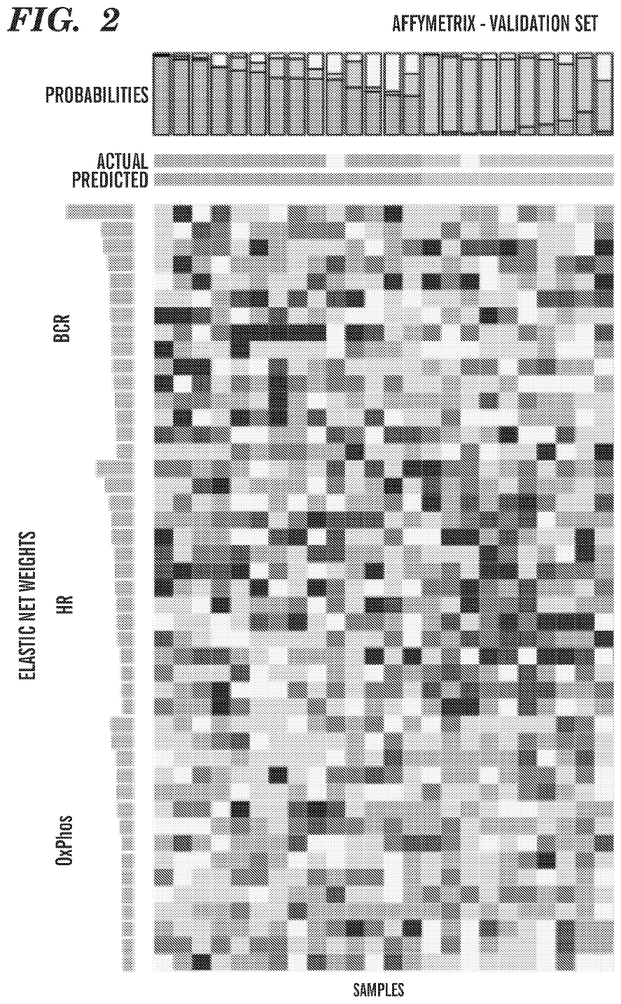

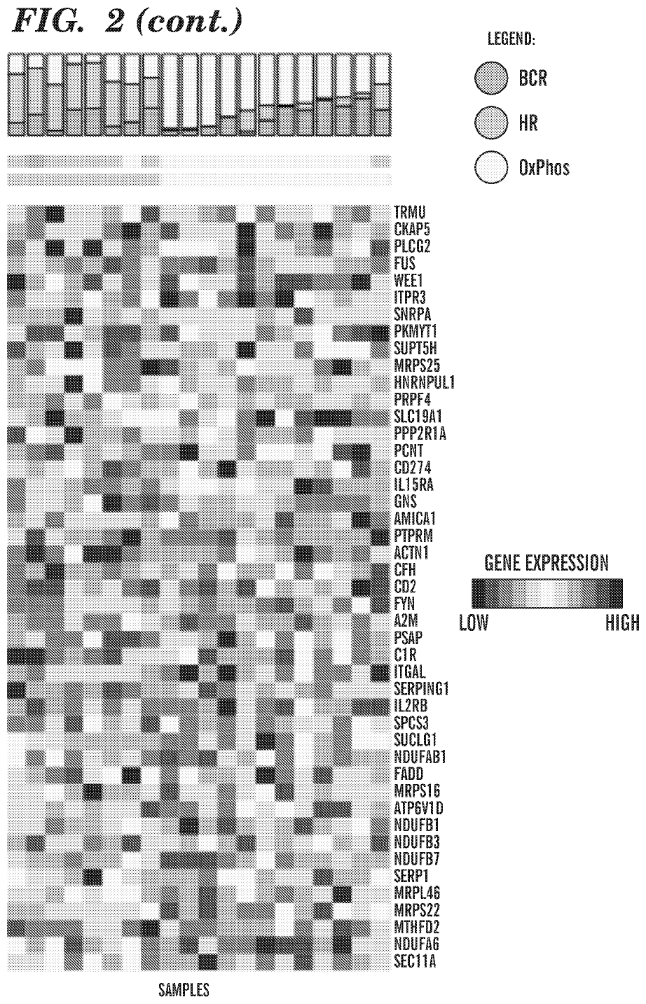

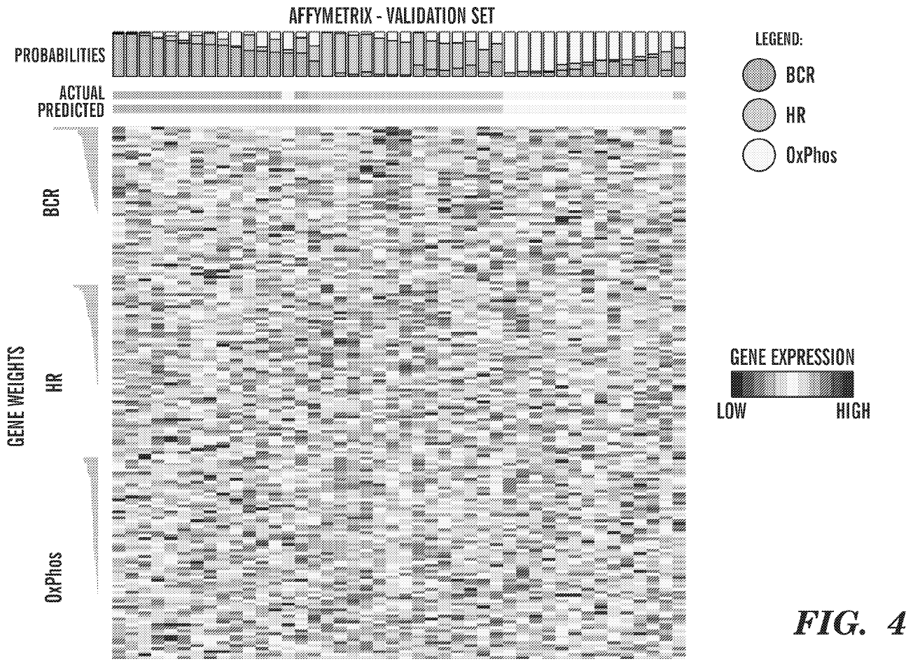

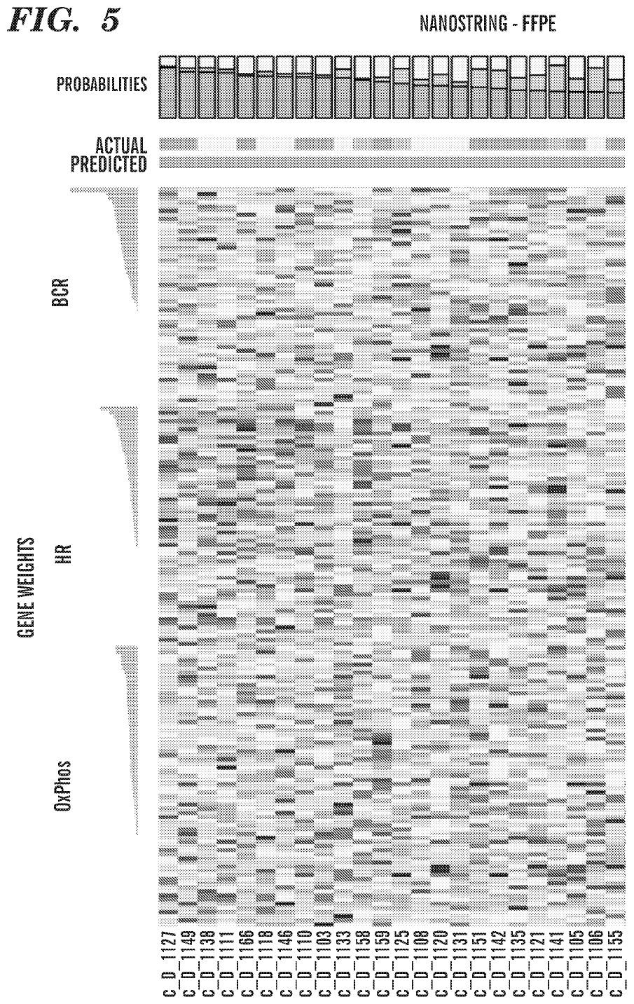

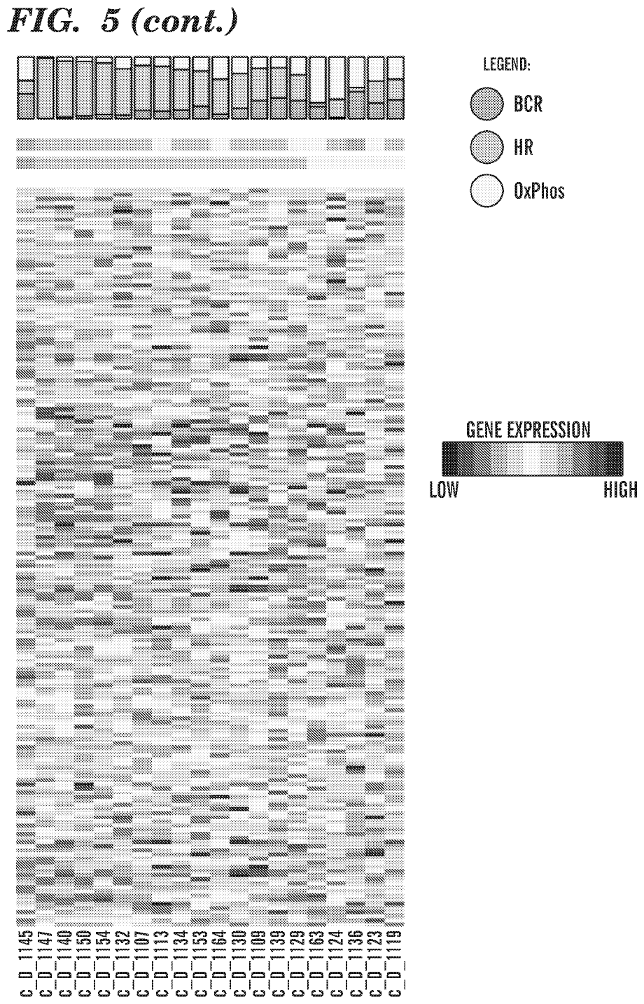

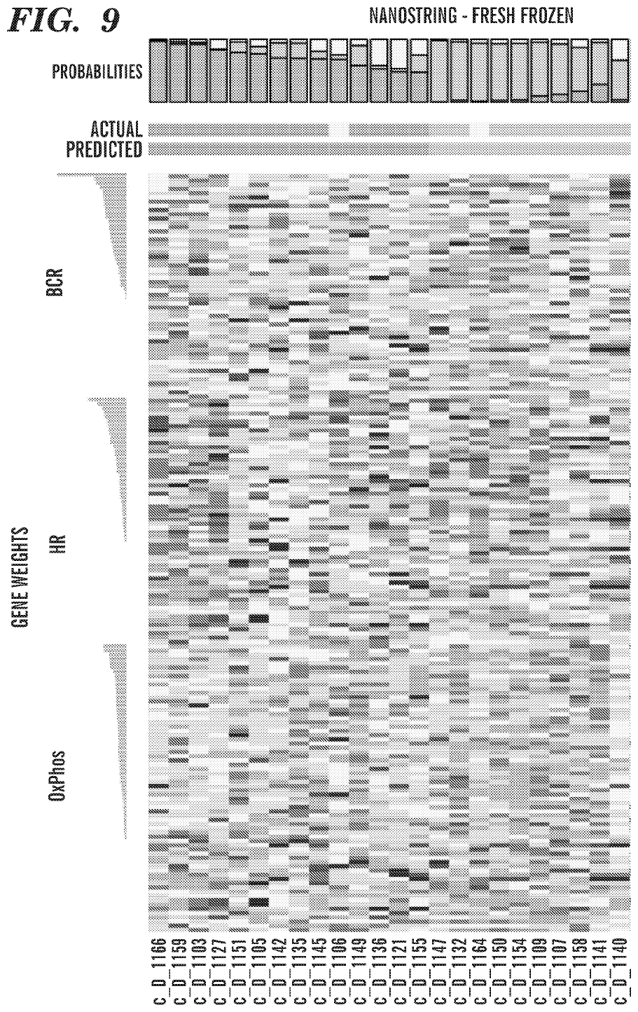

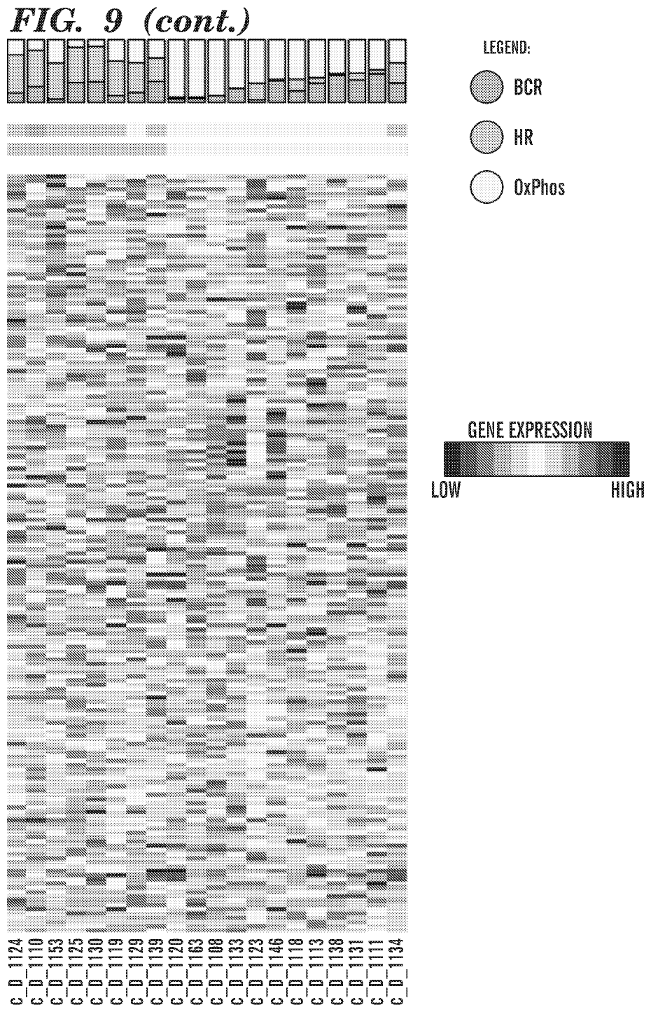

The classification performance was tested on the Nanostring data using our parsimonious model trained on Discovery set I. As shown in Table 7, classification accuracy of 88.6% was achieved in the frozen set and 59.1% in the FFPE data. The heatmaps in FIG. 2 shows the gene expression profiles of the top 15 genes of each class in the Nanostring frozen dataset, with the samples ranked by their class probabilities and grouped by subtypes. FIG. 9 shows the same heatmap with all genes. For comparison, we show the corresponding heatmap for the 44 samples profiled on Affymetrix in FIG. 4 and the ones processed in Nanostring FFPE in FIG. 5.

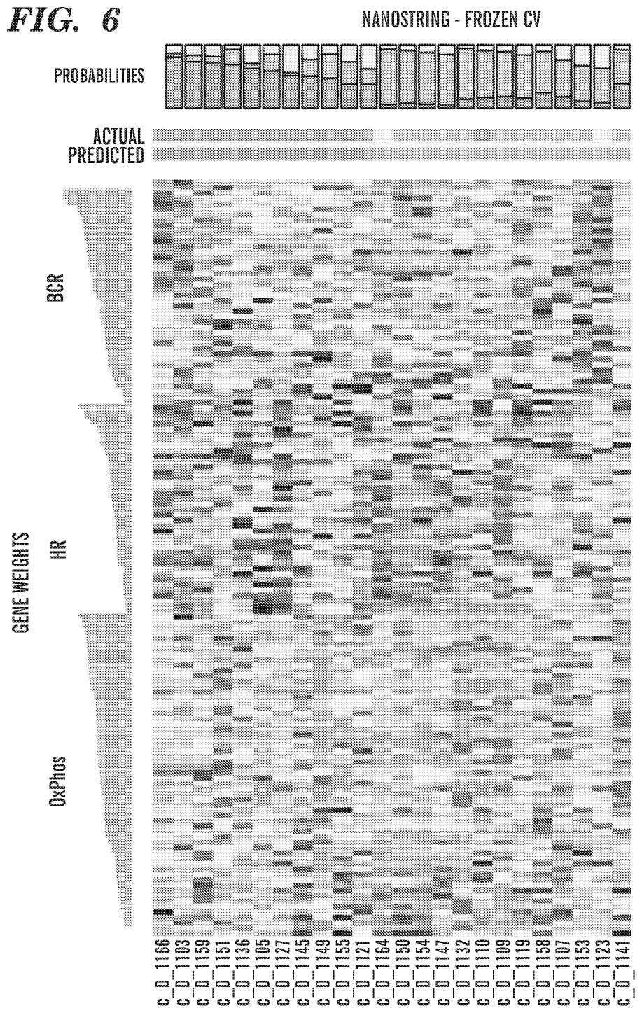

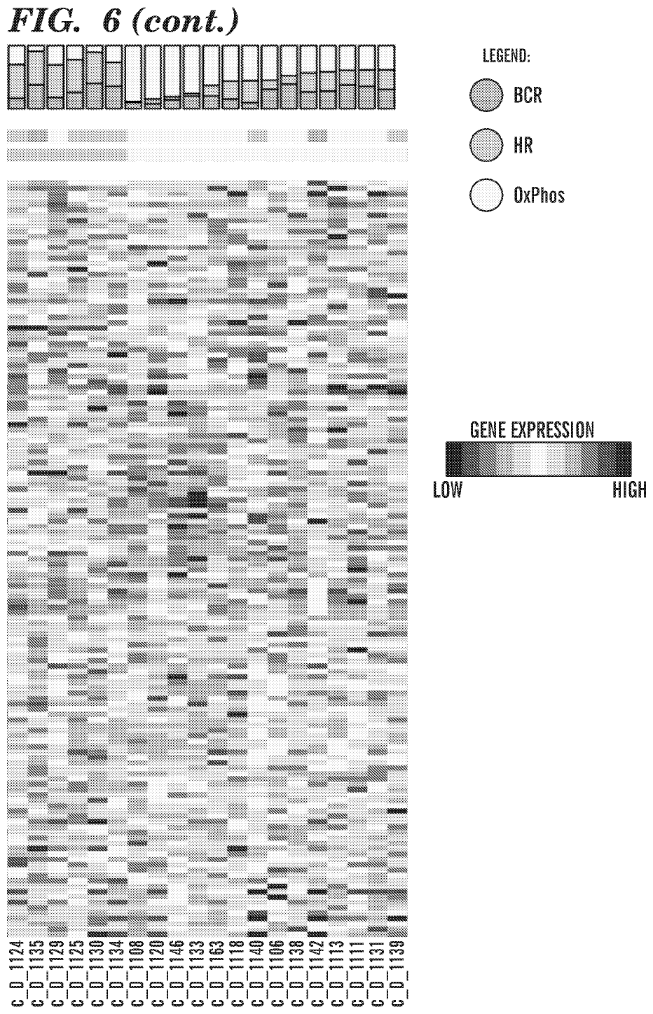

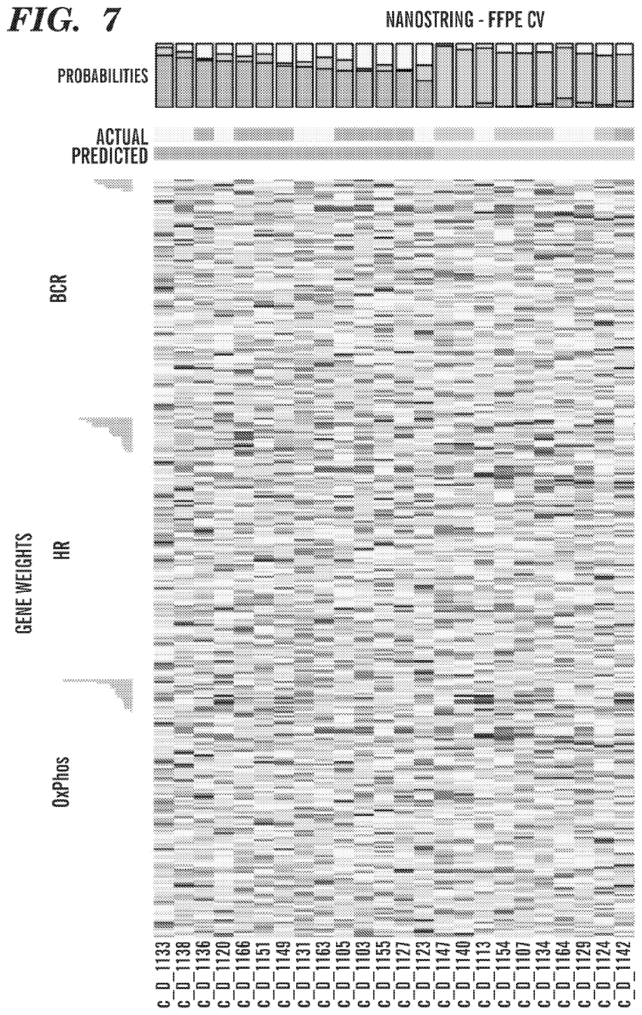

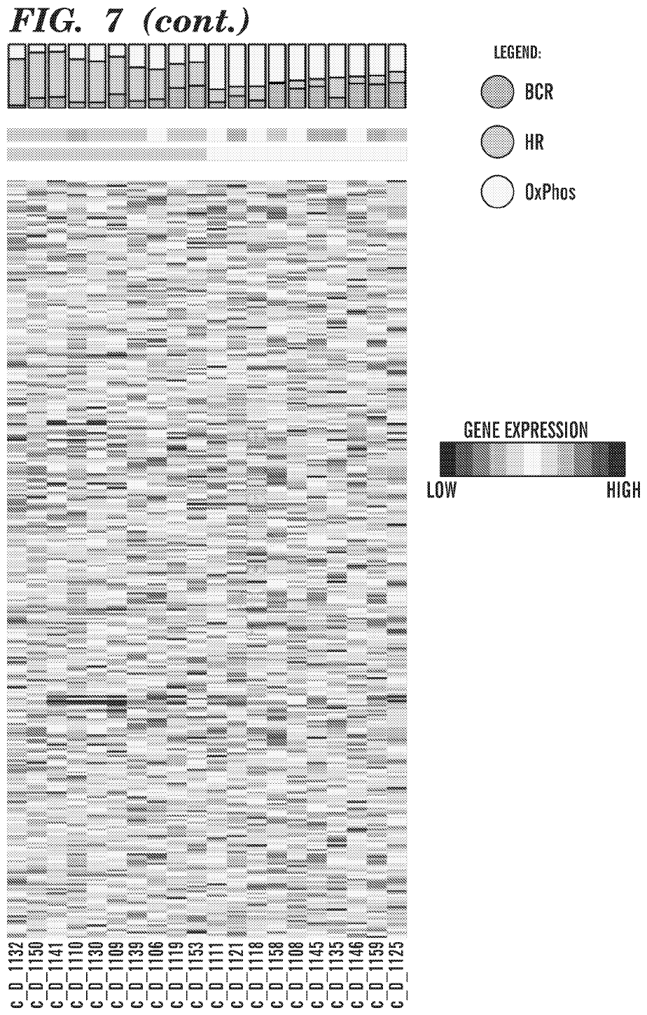

In addition to the classification based on the Affymetrix model leave-one-out cross-validation (LOOCV) was also used within the Nanostring datasets. The models in the LOOCV are trained on 43 samples as opposed to the 141 samples that were used to train the Affymetrix model, so as expected the prediction performance is reduced to 81.8% and 59.9% accuracy (Table 8). The heatmaps are shown in FIG. 6 and FIG. 7. As comparison, the same LOOCV performed within the 44 samples in the Affymetrix discovery set also led to an accuracy of 81.8%.

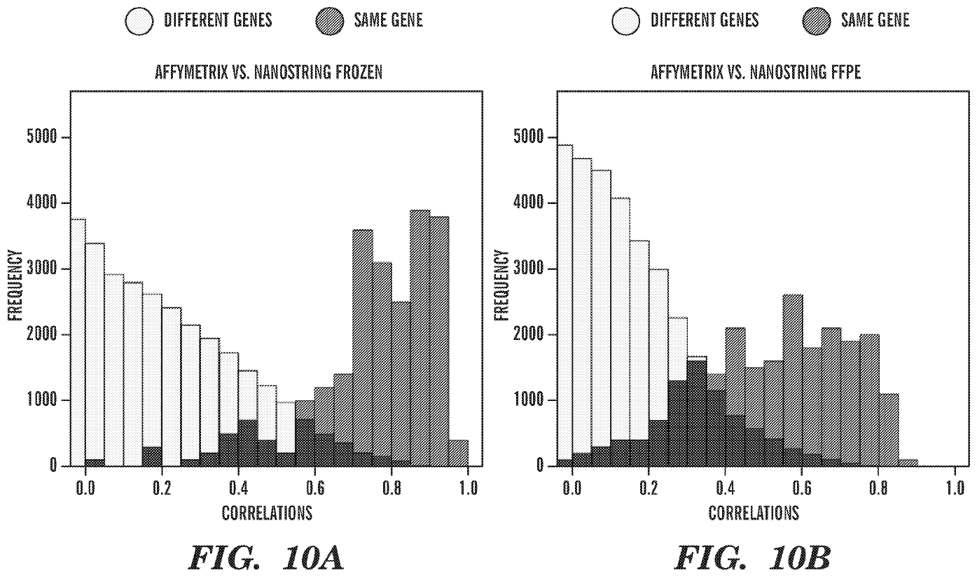

In order to address the discrepancy of accuracies the correlation of probes between the Affymetrix dataset and the two Nanostring sets were looked at. In FIG. 10 is shown the correlations of the same genes in both platforms in blue and as comparison the correlation of different genes across platforms in red to establish a null distribution. For the Nanostring frozen set, the majority of correlations between same genes are above 0.6, with only a few probes not reproducing the Affymetrix signal. In Nanostring FFPE however, there is a much larger overlap between the two groups, reflecting the increased level of noise.

Table 8 Shows Cross-Validation within the Discovery and Validation Sets.

For the discovery 10-fold cross-validation was used, while for the validation sets leave-out-one cross-validation (LOOCV) was used. The first three columns show all the Affymetrix data, while the last two show the prediction performance of the Nanostring data. (ACC: accuracy, SENS: sensitivity, SPEC: specificity).

| | Measurement | | | | |
|---|---|---|---|---|---|
| | | | Validation | Validation | Validation |
| | | | Affymetrix | Nanostring | Nanostring |
| | Discovery I | Discovery II | (44 replicates) | frozen | FFPE |
| | Technology | | | | |
| | Affymetrix | | | Nanostring | |
| Samples | 141 | 72 | 44 | 44 | 44 |
| ACC | 0.965 | 0.918 | 0.818 | 0.818 | 0.591 |
| SENS - BCR | 0.980 | 0.840 | 0.545 | 0.786 | 0.929 |
| SPEC - BCR | 0.956 | 0.979 | 0.939 | 1.000 | 0.600 |
| SENS - HR | 0.905 | 1.000 | 0.947 | 0.875 | 0.688 |
| SPEC - HR | 1.000 | 0.907 | 0.840 | 0.821 | 0.893 |
| SENS - OxP | 1.000 | 0.889 | 0.857 | 0.786 | 0.143 |
| SPEC - OxP | 0.989 | 0.982 | 0.933 | 0.900 | 0.900 |

### Learning Curves for Sample Size Estimation

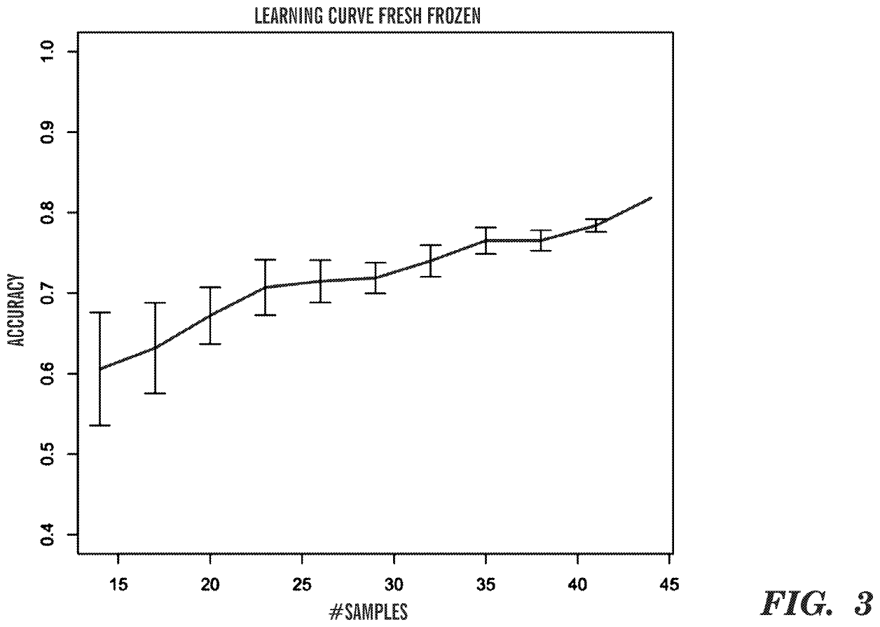

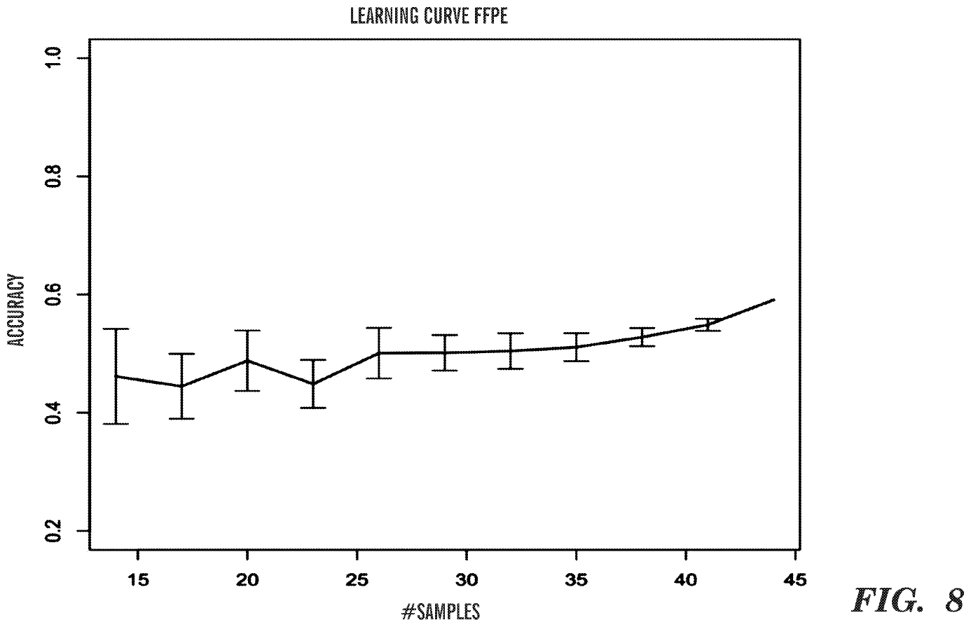

It was determined whether the available sample size was sufficient to achieve maximum prediction accuracy, by carrying out down-sampling experiments to estimate learning curves relating classification accuracy to sample size. In particular, starting from a training set consisting of 13 samples, up to the total number of samples (n=44) in increments of 3, properly stratified datasets were randomly sampled 1000 times for each sample size, and accuracy means and standard deviations were estimated based on leave-one-out cross-validation within each of the sampled datasets. The estimated Accuracy and their corresponding number of compounds for the frozen set is shown in FIG. 3, the one for the FFPE set in FIG. 8, together with linear regression lines fitted on the [sample size; accuracy] pairs. The curve for the frozen samples clearly shows an upward trend, and no indication of “plateauing”, thus suggesting that an increased sample size would significantly improve prediction accuracy.

### Discussion

Even though the selection of the CCC biomarker genes was based on a purely data-driven approach, it resulted in the inclusion of several well-known genes in their respective subtypes. For the HR subtype, genes that reduce the activity of the specific immune system based on T-cells were included. Notable examples were PD-L1, found to be relevant in a host of recent studies (Green et al. 2010; Zitvogel & Kroemer 2012; Herbst et al. 2014); and CTLA4, which restrains the adaptive immune response of T cells towards tumor associated antigens (Mocellin & Nitti 2013). For the BCR subtype the signature most notably includes SYK, a well-known tyrosine kinase that helps to promote survival in hematopoetic malignancies (Friedberg et al. 2010), while the OxPhos portion includes several genes related to oxidative phosphorylation and the electron transport chain such as SUCLG1, NDUFAB1, ATP6V1D, MRPS16. The accompanying hyperenrichment analysis in Table 6 confirmed that the parsimonious biomarker still captures the same pathways that were previously reported (Monti et al. 2005).

It was shown that the reduced set of 141 genes can still capture all three CCC classes and predict them in Affymetrix with accuracies of up to 93.1%, which is surprising and highly significant considering that a random prediction would be expected to yield 33.3% accuracy. The transition to the clinically more relevant Nanostring platform still results in an accuracy of 88.6% in frozen tissues. Interestingly, this loss of prediction accuracy is not equal among all three classes. While the sensitivities of both BCR and HR samples stay in a very similar range, the sensitivity of OxPhos drops to 78.6%.

Interestingly, FIG. 2 shows that the host response portion of the CCC signature appears to be the most robust to the translation from Affytmetrix to Nanostring, which is even more pronounced when going across tissue preservation technologies. The overall CCC accuracy within the FFPE set drops to 59.1%, while the prediction of the HR samples has a sensitivity of 68.8% and a specificity of 89.3%.

From the learning curves in FIG. 3, it can be concluded that 44 samples are not sufficient to achieve maximum prediction performance. All three curves show an upward trend with no ‘plateauing’, suggesting that an increased sample size would indeed lead to increased classification accuracy. This also explains the considerable difference in accuracy between the predictions based on the models trained on the 141-sample discovery set and those based on LOOCV within the validation set, where each model is trained only on 43 samples.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

### REFERENCES

1. Friedberg, J. W. & Fisher, R. I. Diffuse large B-cell lymphoma. Hematol. Oncol. Clin. North Am. 22, 941-52, ix (2008).
2. Monti, S. et al. Molecular profiling of diffuse large B-cell lymphoma identifies robust subtypes including one characterized by host inflammatory response. Blood 105, 1851-1861 (2005).
3. Wright, G. et al. A gene expression-based method to diagnose clinically distinct subgroups of diffuse large B cell lymphoma. Proc. Natl. Acad. Sci. 100, 9991-9996 (2003).
4. Basso, K. & Dalla-Favera, R. Germinal centres and B cell lymphomagenesis. Nat. Rev. Immunol. 15, 172-184 (2015).
5. Lenz, G. & Staudt, L. M. Aggressive Lymphomas. N Engl. J. Med. 362, 1417-1429 (2010).
6. Scott, D. W. et al. Determining cell-of-origin subtypes of diffuse large B-cell lymphoma using gene expression in formalin-fixed paraffin-embedded tissue. Blood 123, 1214-7 (2014).
7. Caro, P. et al. Metabolic Signatures Uncover Novel Targets in Molecular Subsets of Diffuse Large B Cell Lymphoma. Cancer Cell 22, 547-560 (2012).
8. Chen, L. et al. SYK Inhibition Modulates Distinct PI3K/AKT-Dependent Survival Pathways and Cholesterol Biosynthesis in Diffuse Large B Cell Lymphomas. Cancer Cell 23, 826-838 (2013).
9. Chen, L. et al. SYK-dependent tonic B-cell receptor signaling is a rational treatment target in diffuse large B-cell lymphoma. Blood 111, 2230-2237 (2008).
10. Polo, J. M. et al. Transcriptional signature with differential expression of BCL6 target genes accurately identifies BCL6-dependent diffuse large B cell lymphomas. Proc. Natl. Acad. Sci. U.S.A. 104, 3207-12 (2007).
11. Geiss, G. K. et al. Direct multiplexed measurement of gene expression with color-coded probe pairs. Nat. Biotechnol. 26, 317-25 (2008).
12. Monti, S. et al. Molecular profiling of diffuse large B-cell lymphoma identifies robust subtypes including one characterized by host inflammatory response. Blood 105, 1851-61 (2005).
13. Monti, S. et al. Integrative Analysis Reveals an Outcome-Associated and Targetable Pattern of p53 and Cell Cycle Deregulation in Diffuse Large B Cell Lymphoma. Cancer Cell 22, 359-372 (2012).
14. Smyth, G. Limma: linear models for microarray data. 397-420 (2005).
15. Subramanian, A. et al. Gene set enrichment analysis: a knowledge-based approach for interpreting genome-wide expression profiles. Proc. Natl. Acad. Sci. U.S.A 102, 15545-50 (2005).
16. Irizarry, R. A. Exploration, normalization, and summaries of high density oligonucleotide array probe level data. Biostatistics 4, 249-264 (2003).
17. Dai, M. et al. Evolving gene/transcript definitions significantly alter the interpretation of GeneChip data. Nucleic Acids Res. 33, e 175 (2005).
18. Waggott, D. et al. NanoStringNorm: an extensible R package for the pre-processing of NanoString mRNA and miRNA data. Bioinformatics 28, 1546-8 (2012).
19. Kasprzyk, A. BioMart: driving a paradigm change in biological data management. Database (Oxford). 2011, bar049 (2011).
20. Hui Zou, T. H. Regularization and variable selection via the Elastic Net. at <http://citeseerx.ist.psu.edu/viewdoc/summary?doi=10.1.1.124.4696>
21. Breiman, L. Random Forests. Mach. Learn. 45, 5-32 (2001).
22. Hastie, T., Tibshirani, R., Narasimhan, B. & Chu, G. pamr: Pam: prediction analysis for microarrays. (2011).
23. Tibshirani, R. Regression Shrinkage and Selection Via the Lasso. at <http://citeseer.ist.psu.edu/viewdoc/summary?doi=10.1.1.35.7574>
24. Green, M. R. et al. Integrative analysis reveals selective 9p24.1 amplification, increased PD-1 ligand expression, and further induction via JAK2 in nodular sclerosing Hodgkin lymphoma and primary mediastinal large B-cell lymphoma. Blood 116, 3268-77 (2010).
25. Zitvogel, L. & Kroemer, G. Targeting PD-1/PD-L1 interactions for cancer immunotherapy. Oncoimmunology 1, 1223-1225 (2012).
26. Herbst, R. S. et al. Predictive correlates of response to the anti-PD-L1 antibody MPDL3280A in cancer patients. Nature 515, 563-567 (2014).
27. Mocellin, S. & Nitti, D. CTLA-4 blockade and the renaissance of cancer immunotherapy. Biochim. Biophys. Acta 1836, 187-96 (2013).
28. Chen, L. et al. SYK-dependent tonic B-cell receptor signaling is a rational treatment target in diffuse large B-cell lymphoma. Blood 111, 2230-7 (2008).

What is claimed is:

1. A method of treating a subject for cancer, the method comprising:
a. assaying a sample from a cancer cell of the subject, for levels of gene expression of at least four genes from genes of SEQ ID NOs 1-141 or a subset thereof;
b. normalizing the assayed levels of gene expression with a control;
c. identifying the genes whose assayed levels of expression are upregulated;
d. administering a therapeutic regimen for the treatment of cancer of the BCR subtype if the expression of at least two of the group consisting of TRMU, CKAP5, PLCG2, FUS, WEE1, ITPR3, SNRPA, PKMYT1, and SUPT5H are identified to be upregulated; or
e) administering a therapeutic regimen for the treatment of cancer of the Host Receptor subtype if the expression of at least two genes of the group consisting of PD-L1, CTLA4, IL15RA, GNS, PTPRM, AMICA, CFH, CD2, ITGAL, ACTN1, A2M and IL2RB are identified to be upregulated; or
f) administering a therapeutic regimen for the treatment of cancer of the OxPhos subtype if the expression of at least two of the group consisting of SPCS3, SUCLG1, NDUFAB1, FADD, MRPS16, ATP6V1D, NDUFB1, NDUFB3, SEC11A and PARK7 are identified to be upregulated.

2. The method of claim 1, wherein the gene expression is assayed by measuring the nucleic acid encoded by the gene or by measuring or detecting a protein encoded by the gene, using qPCR, microarray, nCounter® analysis system, by immunoassay, targeted mass spectrometry, or immunolabeling.

3.-5. (canceled)

6. The method of claim 1, wherein step (c) is by linear combination of the normalized levels of gene expression obtained from step (b).

7. (canceled)

8. The method of claim 1, wherein step (c) comprises applying a classifier, wherein the classifier has been trained with training data from a plurality of cancer patients, wherein the training data comprise for each of the plurality of cancer patients (a) weighted gene expression level of at least the plurality of genes for which the expression levels are assayed including said at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7 and (b) information with respect to subtype of DLBCL based on the weighted gene expression level.

9. The method of claim 8, wherein the classifier is selected from Elastic net, Random Forest, and Shrunken centroids.

10. The method of claim 1, wherein the upregulation is relative to the levels of gene expression in a sample from a non-cancer cell.

11. (canceled)

12. The method of claim 1, wherein the cancer is DLBCL.

13. The method of claim 12, wherein the cancer is relapsed or refractory to treatment with a CHOP or rituximab(R)/CHOP treatment regimen.

14. The method of claim 1, wherein the cancer cell is a cell obtained from tumor biopsy, frozen cancer tissue, or paraffin-embedded cancer tissue.

15.-25. (canceled)

26. A kit comprising a plurality of probes for determining levels of gene expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H, or at least {two} genes selected from group consisting of PD-L1, CTLA4, IL15RA, GNS, PTPRM, AMICA, CFH, CD2, ITGAL, ACTN1, A2M and IL2RB, or at least three genes selected from group consisting of SPCS3, SUCLG1, NDUFAB1, FADD, MRPS16, ATP6V1D, NDUFB1, NDUFB3, SEC11A and PARK7.

27.-30. (canceled)

31. The kit of claim 26, wherein the probes are nucleic acid primers for amplification of the genes.

32. The kit of claim 26, wherein each probe in the plurality of probes comprises a target specific sequence that hybridizes to no more than one gene under stringent hybridization conditions.

33. The kit of claim 26, wherein the plurality of probes comprises probe pairs to detect the expression of genes of SEQ ID NOs 1-141 or a subset thereof, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC 11A and PARK7, wherein each probe in the probe pair comprises a target specific sequence that hybridizes to no more than one gene under stringent hybridization conditions, and wherein the target-specific sequences in each pair hybridize to different regions of the same gene.

34. The kit of claim 26, wherein a probe molecule for each gene comprises a label.

35. The kit of claim 26, wherein the probes are immobilized on a solid support.

36.-41. (canceled)

42. A computer readable medium or computer program product comprising a classifier that predicts the DLBCL-subtype, based on weighted expression of genes of SEQ ID NOs 1-141 or a subset thereof in a sample from a subject having or suspected of having DLBCL, wherein the subset comprises at least four genes selected from group consisting of TRMU, CKAP5, PLCG2, FUS, WEE1, ITPR3, SNRPA, PKMYT1, and SUPT5H and at least {two} genes selected from group consisting of PD-L1, CTLA4, IL15RA, GNS, PTPRM, AMICA, CFH, CD2, ITGAL, ACTN1, A2M and IL2RB and at least three genes selected from group consisting of SPCS3, SUCLG1, NDUFAB1, FADD, MRPS16, ATP6V1D, NDUFB1, NDUFB3, SEC11A and PARK7, said classifier having been trained by in silico analysis and classification algorithms.

43. The computer readable medium or computer program product of claim 42, wherein the classifier has been trained with training data from a plurality of DLBCL patients, wherein the training data comprise for each of the plurality of DLBCL patients (a) weighted gene expression level of at least the plurality of genes for which the expression levels are measured including said at least four genes selected from group consisting of TRMU, CKAP5, PLCG2, FUS, WEE1, ITPR3, SNRPA, PKMYT1, and SUPT5H, and at least {two} genes selected from group consisting of PD-L1, CTLA4, IL15RA, GNS, PTPRM, AMICA, CFH, CD2, ITGAL, ACTN1, A2M and IL2RB, and at least three genes selected from group consisting of SPCS3, SUCLG1, NDUFAB1, FADD, MRPS16, ATP6V1D, NDUFB1, NDUFB3, SEC11A and PARK7 and (b) information with respect to subtype of DLBCL based on the weighted gene expression level.

44. The computer readable medium or computer program product of claim 42, wherein said classifier is trained by one or more algorithms selected from the group consisting of dual ensemble, generalized simulated annealing, T-filter, CORG, CORG combined with support vector machine, dual bagging, single and pairs, forward learning, Laplacian based learning and learning method based on network perturbation amplitude.

45. The computer readable medium or computer program product of claim 42, wherein said classifier is trained with at least the data in the Gene Expression Omnibus datasets GSE2109, GSE 10245, GSE1 8842 and GSE37745.

46.-53. (canceled)

54. The method of claim 1, wherein the subset comprises at least four genes selected from group consisting of TRMU, CKAP5, PLCG2, FUS, WEE1, ITPR3, SNRPA, PKMYT1, and SUPT5H and/or at least two genes selected from group consisting of PD-L1, CTLA4, IL15RA, GNS, PTPRM, AMICA, CFH, CD2, ITGAL, ACTN1, A2M and IL2RB and/or at least three genes selected from group consisting of SPCS3, SUCLG1, NDUFAB1, FADD, MRPS16, ATP6V1D, NDUFB1, NDUFB3, SEC11A and PARK7.


SEQUENCE LISTING (141 sequences)

SEQ ID NO: 1 - DNA, 3926 bp, Homo sapiens
aattcttggaagaggagaactggacgttgtgaacagagttagctggtaaatgtcctcttaaaagatccaaaaaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattagatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagaatcaatcataaagtgcacacctttattcagaactttagtattaaatcagttctcaatttcattttttatgtattgttttactcctttttattcatacgtaaaattttggattaatttgtgaaaatgtaattataagctgagaccggtggctctctt

SEQ ID NO: 2 - DNA, 663 bp, Homo sapiens
agtgcatcctgggttggcgtagccatggcgtctcgtgtcctttcagcctatgtcagccgcctgcccgcggcctttgcgccgctgccccgggtccggatgctggccgtggcccggcctctcagcactgctctctgctccgcggggacccagacgaggctcgggactttgcagccggccttagtgctcgcgcaggttcctggtagagttacacagttgtgccgccagtatagcgacatgcctcctttgacgttagagggcatccaggaccgtgttctttacgtattgaaactctatgacaagattgacccagagaagctttcagtaaattctcattttatgaaagacctgggcttagacagtttggaccaagtggagattatcatggccatggaagacgaatttgggtttgaaattcctgatatagatgctgaaaagttaatgtgtccacaagaaattgtagattacattgcagataagaaagatgtatatgaataaagtatcagaccctttggctttgctgagagaggactcagatgatagtgacgaatgtctggcagtgaggacacattttggcattcttgctgactctgacagagtgattctgatggacttgtatttaaattgtatgtgttttactctttgaaaataaatctataaaaccaa

SEQ ID NO: 3 - DNA, 420 bp, Homo sapiens
ggggtctggacgcaacggcggcgggacgatgaacgcccctccagccttcgagtcgttcttgctcttcgagggcgagaagaagatcaccattaacaaggacaccaaggtacccaatgcctgtttattcaccatcaacaaagaagaccacacactgggaaacatcattaaatcacaactcctaaaagacccgcaagtgctatttgctggctacaaagtcccccaccccttggagcacaagatcatcatccgagtgcagaccacgccggactacagcccccaggaagcctttaccaacgccatcaccgacctcatcagtgagctgtccctgctggaggagcgctttcgggtggccataaaagacaagcaggaaggaattgagtaggggccagagggggctctgctcggcctgtgagccccgtt

SEQ ID NO: 4 - DNA, 4974 bp, Homo sapiens
acacctccctccccgcctgccagtgtcaccagcctgttgcctctgtgagaaagtaccactgtaagaggccaaagggcatgatcattttcctctttcaccctgtctaggttgccagcaaatcccacgggcctcctgacgctgcccctggggccacaggtccctcgagtgctggaaggatgaaggattcctgcatcactgtgatggccatggcgctgctgtctgggttctttttcttcgcgccggcctcgagctacaacctggacgtgcggggcgcgcggagcttctccccaccgcgcgccgggaggcactttggataccgcgtcctgcaggtcggaaacggggtcatcgtgggagctccaggggaggggaacagcacaggaagcctctatcagtgccagtcgggcacaggacactgcctgccagtcaccctgagaggttccaactatacctccaagtacttgggaatgaccttggcaacagaccccacagatggaagcattttgtttgctgctgttcagttttccacaagctacaaaacagaatttgatttctcagattatgttaaacggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacatttgagaagctgaaagatctattcactgagctgcagaagaagatctatgtcattgagggcacaagcaaacaggacctgacttccttcaacatggagctgtcctccagcggcatcagtgctgacctcagcaggggccatgcagtcgtgggggcagtaggagccaaggactgggctgggggctttcttgacctgaaggcagacctgcaggatgacacatttattgggaatgaaccattgacaccagaagtgagagcaggctatttgggttacaccgtgacctggctgccctcccggcaaaagacttcgttgctggcctcgggagcccctcgataccagcacatgggccgagtgctgctgttccaagagccacagggcggaggacactggagccaggtccagacaatccatgggacccagattggctcttatttcggtggggagctgtgtggcgtcgacgtggaccaagatggggagacagagctgctgctgattggtgccccactgttctatggggagcagagaggaggccgggtgtttatctaccagagaagacagttggggtttgaagaagtctcagagctgcagggggaccccggctacccactcgggcggtttggagaagccatcactgctctgacagacatcaacggcgatgggctggtagacgtggctgtgggggcccctctggaggagcagggggctgtgtacatcttcaatgggaggcacggggggcttagtccccagccaagtcagcggatagaagggacccaagtgctctcaggaattcagtggtttggacgctccatccatggggtgaaggaccttgaaggggatggcttggcagatgtggctgtgggggctgagagccagatgatcgtgctgagctcccggcccgtggtggatatggtcaccctgatgtccttctctccagctgagatcccagtgcatgaagtggagtgctcctattcaaccagtaacaagatgaaagaaggagttaatatcacaatctgtttccagatcaagtctctcatcccccagttccaaggccgcctggttgccaatctcacttacactctgcagctggatggccaccggaccagaagacgggggttgttcccaggagggagacatgaactcagaaggaatatagctgtcaccaccagcatgtcatgcactgacttctcatttcatttcccggtatgtgttcaagacctcatctcccccatcaatgtttccctgaatttctctctttgggaggaggaagggacaccgagggaccaaagggcgggcaaggacataccgcccatcctgagaccctccctgcactcggaaacctgggagatcccttttgagaagaactgtggggaggacaagaagtgtgaggcaaacttgagagtgtccttctctcctgcaagatccagagccctgcgtctaactgcttttgccagcctctctgtggagctgagcctgagtaacttggaagaagatgcttactgggtccagctggacctgcacttccccccgggactctccttccgcaaggtggagatgctgaagccccatagccagatacctgtgagctgcgaggagcttcctgaagagtccaggcttctgtccagggcattatcttgcaatgtgagctctcccatcttcaaagcaggccactcggttgctctgcagatgatgtttaatacactggtaaacagctcctggggggactcggttgaattgcacgccaatgtgacctgtaacaatgaggactcagacctcctggaggacaactcagccactaccatcatccccatcctgtaccccatcaacatcctcatccaggaccaagaagactccacactctatgtcagtttcacccccaaaggccccaagatccaccaagtcaagcacatgtaccaggtgaggatccagccttccatccacgaccacaacatacccaccctggaggctgtggttggggtgccacagcctcccagcgaggggcccatcacacaccagtggagcgtgcagatggagcctcccgtgccctgccactatgaggatctggagaggctcccggatgcagctgagccttgtctccccggagccctgttccgctgccctgttgtcttcaggcaggagatcctcgtccaagtgatcgggactctggagctggtgggagagatcgaggcctcttccatgttcagcctctgcagctccctctccatctccttcaacagcagcaagcatttccacctctatggcagcaacgcctccctggcccaggttgtcatgaaggttgacgtggtgtatgagaagcagatgctctacctctacgtgctgagcggcatcggggggctgctgctgctgctgctcattttcatagtgctgtacaaggttggtttcttcaaacggaacctgaaggagaagatggaggctggcagaggtgtcccgaatggaatccctgcagaagactctgagcagctggcatctgggcaagaggctggggatcccggctgcctgaagcccctccatgagaaggactctgagagtggtggtggcaaggactgagtccaggcctgtgaggtgcagagtgcccagaactggactcaggatgcccagggccactctgcctctgcctgcattctgccgtgtgccctcgggcgagtcactgcctctccctggccctcagtttccctatctcgaacatggaactcattcctgcctgtctcctttgcaggctcatagggaagacctgctgagggaccagccaagagggctgcaaaagtgagggcttgtcattaccagacggttcaccagcctctcttggtttccttccttggaagagaatgtctgatctaaatgtggagaaactgtagtctcaggacctagggatgttctggccctcacccctgccctgggatgtccacagatgcctccaccccccagaacctgtccttgcacactcccctgcactggagtccagtctcttctgctggcagaaagcaaatgtgacctgtgtcactacgtgactgtggcacacgccttgttcttggccaaagaccaaattccttggcatgccttccagcaccctgcaaaatgagaccctcgtggccttccccagcctcttctagagccgtgatgcctccctgttgaagctctggtgacaccagcctttctcccaggccaggctccttcctgtcttcctgcattcacccagacagctccctctgcctgaaccttccatctcgccacccctccttccttgaccagcagatcccagctcacgtcacacttggttgggtcctcacatctttcacacttccaccagcctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgatgcctcatgatatgtcagggcgtgggacatctagtaggtgcttgacataatttcactgaattaatgacagagccagtgggaagatacagaaaaagaggggctgggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattgcttgaacctgggaggtggaggttgcagtgagccaagattgcgccattgcactccagcctgggcaacacagcgagactccgtctcaaggaaaaaataaaaataaaaagcgggcacgggcccgtgacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataaatgtgaaattctgtccaaaaaaaaaaaaaaaaaa

SEQ ID NO: 5 - DNA, 2173 bp, Homo sapiens
aacaaaagtgtctgagaaagccagagtgtgaccacgcggtttctatgctccaagggatttatttcagtggtaggtggagcagctctgtggagcacatggctaatgataatggattgactgaccagaagcaggagaggacctagcagaagccggaggtcaagctgccccaagactgggggacagcctgggtgaagactctggacctcatgaccatgtatattccagagggggatctaaagagccccagaaattcacaaaggaccagtcctgcctctgttgtagaagacatgtggtgtatataaagtttgtgatcgttggcggaaattttggaatttagataatgggctgtgtgcaatgtaaggataaagaagcaacaaaactgacggaggagagggacggcagcctgaaccagagctctgggtaccgctatggcacagaccccacccctcagcactaccccagcttcggtgtgacctccatccccaactacaacaacttccacgcagccgggggccaaggactcaccgtctttggaggtgtgaactcttcgtctcatacggggaccttgcgtacgagaggaggaacaggagtgacactctttgtggccctttatgactatgaagcacaggcagaagagtggtactttggaaaacttggccgaaaagatgctgagcgacagctattgtcctttggaaacccaagaggtacctttcttatccgcgagagtgaaaccaccaaaggtgcctattcactttctatccgtgattgggatgatatgaaaggagaccatgtcaaacattataaaattcgcaaacttgacaatggtggatactacattaccacccgggcccagtttgaaacacttcagcagcttgtacaacattactcagagaaagctgatggtttgtgttttaacttaactgtgattgcatcgagttgtaccccacaaacttctggattggctaaagatgcttgggaagttgcacgtcgttcgttgtgtctggagaagaagctgggtcaggggtgtttcgctgaagtgtggcttggtacctggaatggaaacacaaaagtagccataaagactcttaaaccaggcacaatgtcccccgaatcattccttgaggaagcgcagatcatgaagaagctgaagcacgacaagctggtccagctctatgcagtggtgtctgaggagcccatctacatcgtcaccgagtatatgaacaaaggaagtttactggatttcttaaaagatggagaaggaagagctctgaaattaccaaatcttgtggacatggcagcacaggtggctgcaggaatggcttacatcgagcgcatgaattatatccatagagatctgcgatcagcaaacattctagtggggaatggactcatatgcaagattgctgacttcggattggcccgattgatagaagacaatgagtacacagcaagacaaggtgcaaagttccccatcaagtggacggcccccgaggcagccctgtacgggaggttcacaatcaagtctgacgtgtggtcttttggaatcttactcacagagctggtcaccaaaggaagagtgccatacccaggcatgaacaaccgggaggtgctggagcaggtggagcgaggctacaggatgccctgcccgcaggactgccccatctctctgcatgagctcatgatccactgctggaaaaaggaccctgaagaacgccccacttttgagtacttgcagagcttcctggaagactactttaccgcgacagagccccagtaccaacctggtgaaaacctgtaaggcccgggtctgcggagagaggccttgtcccagaggctgccccacccctccccattagctttcaattccgtagccagctgctccccagcagcggaaccgcccaggatcagattgcatgtgactctgaagctgacgaacttccatggccctcattaatgacacttgtccccaaatccgaacctcctctgtgaagcattcgagacagaaccttgttatttctcagactttggaaaatgcattgtatcgatgttatgtaaaaggccaaacctctgttcagtgtaaatagttactccagtgccaacaatcctagtgctttcctttttttaaaaatgcaaatcctatgtgattttaactctgtcttcacctgattcaact

SEQ ID NO: 6 - DNA, 32767 bp, Homo sapiens
cgggagttcgagaccagccgtcaggctctactaaaaatacaaaattagccgggcgtggtggcgcatgcctgtaatcccagctactcaggaggctgaggcaggagaatcgcttgaacccaggaggcagaggttgcggtgagctgagattgcgccattgcactccatcctgggcaatgagagcgaaactccatctccaaaaaaaaaaaaaaaatgctaaatagctaatgtttacgtgcaaaattatttcctaagaacacataactaactgtaattgcttggtcagaagagatacaaaagattaaggcttttgatgcacactctcaaatcaaattccagaaattctctgggtcttctgtgaatatgcctgaacagaaacatgagcagatattatgattccagatgttaaatatggacttttcaatccctttgtgataaacccattctgcaaactggagtagcccaacctgggcccatcccatgtctttgaaatcctgagtgattgctcacacctttgcagctgtgaggatgggtaacaggggcccatacacctgggcccagagcgaggagatatgtcttagaggagctcaagcagcaggctgtggtggtggtgtcagcatcagagccctagtgcatcctgtctggacagacttcagagagtcagtcaaaacacgacttcagagagtcagtcagaacacagagcaacagtttgaccatctttgctttacactgggcttaggtcggtttctcaggttagcaaaaaattaaattctcagtatttccagtttaacaatatgatatttaagaatatgatatttggagttttttgtttgtttgcttgtttgtttttgtttttttaacatataaacttttaaaataaccacattaggctgggcgctgtggctcacgcctgtaatcccagcactatgggaggccgaggcgggcagatcatgaggtcaaagatcaagaccatcttggccaacatggtgaaaccccatctctattaaaaatacaaaaattagctgggcatggtggcatgtgcctgtgaagtcacatactcaggaggctgaggcaggagagttgcttgaacccgggagctagaggtagcagtgagccaagatcgcaccactacgctcccacctggtgacagagcaagactccgtctcaaaaaaaaaactgcattaattgtccatgtgtgccatgacttagtgtgtctaacatttcactcccataaacaatccaccatggacaattctgtgcataaatcttgttctcacttcatattatttccttgaagtggaaaagatatgaatctatttaagactcttgatatatattaacaaattattttcctaagaagttattccatcttccactctaggatctgtttttgaattgtacattgtgcttcagtgatttaactattgcatttttacagacagttttaacacctggcagactagcctttcttcatttcttttcttataattattttacctcttctcacctgtttatttttctcagcaaaactctgcataatttgttaagtttccccaaaatcaccatgacatttcaattgaaatgtggttaaacttagaattaattcaggaaggtttcacatctttacctcatccaaactttcatcctagctcatggtctgtctcaagtattttcttttcttttgtgttcttgtagaatagaatctctttctgctatattttctgactgataatggctggcatatttttcttctgtaaaaccacattgacaccaacatttgttttatttcagttaagatttattgtgagttgatagtcttcttaatgttgtcaaataaaggttaataccatggaaatctgatcagttaagaaaaatatatgtagactgataaaaaactataagaaagaattaatctattaatctaatagattaatccaagtaacatgaatcctctggaatatatgaggaaaataactcattattttcttagtataattgattttttaaatcttttaaatttttttaaatttatttttattttttgaggcagggtctcactctgttgtccaggctggagtacagaggcacaatctgggcttattgcagccttgacctcctgggctcaagcagacctcgtgcctcagcctcccaagtagctgggactgtaagcacatgcccctacgcccggctaatttttgtattttttttttttatagagatggtgtctcactgtgttgcccaggctagtcctgagctcctgggctcaagcagtcctccctccttggcctcccaaagtgctgggattataggcatgagccactgcacccagcctactgtccttttaaaaagatgtaattgagaaaaattgcatatatttatggtgtacaatgtgttgttttgatatatgtacacattgtggaatgattaaaccaaggtaattagcatatccatcacctcacacatatcatttgttgtgatgggaatgttaaaaatctactctcttagcaattttctactaaataatgcattgctattaactgtagtcaccaggatgcacagtagatctccagaacttattccaatggcatataattttttaaatttttatttgcatatttgttttctatcctaattccctactgatcttactttaacaggtaaaatcctcttagattttctaggattctatgatcagcaaataaggatgttttcttcttctaatagttatgccatggtgtctgattattttctttattatactttttagaaaaccctatgcagtgttaaagaatgctggctacaggcagtcaggtcttatttccagatataaatgcaacaaacgcttccctggttagatgtgatgttggctgttggtttgggatggatagtctctgtcatgttaaggaagcagccttctggttttgtttatcagcactcatattaggaatgggagttatattttacttaatgcctattgtccagttgttgaggtgatgtattatttgcttttaattttctcttttgccctttagatatgatttatgttcagttaattgattgcctaatattgagatatcctcagataaattcatattgaccatgtcatggtagacttaatataccattaaattctgtttgggaaaaaaaatctatttgcaaaaaaatctatttgctggggttatatagaattttgcagttattttactaagactagctttttctcaatatgcattgttaggttagtgaaagagggtttcactgacttcacagagggaatgagttgcctctgtctttttttattttttttgagacagagtcacactgtgtcgcccaggctggagtgcggtggtgccacctcagctcactgcaacttccgcctcccgggttcaagcagttcttgtgccttagcctcctgagcagctgggattacaggcacgtgccaccatgcccggctaatttttgtgtttttagtagagacgaggttttgccatgttggccaggctggtctcaaactcctgacttcaggtggtccgcccacctcagcctcccaaagtgctgggattacaggtgtgaaccacagcgcccggccacctctttttactgtgttacagcagacttcaggacaagatcttaacttgttccttgctgaatagacagacacccttaagggagggaggtattaggccctgacaagctgcagccatattatattatattttgcagacttttattatcttcttaagcaggaaacatggctctttcatttagctttggtggtcacattaagtcttttttttttttttttttttttttttttttgagatggagtcttgctctgtcgcccaggctggagtgcagtggcgcgatctcggctcactgcaagctccgcctcccgggttcacaccatttgcctgcatcagcctcctgagtagctgggactacaggcccccgccaccatacccagctaattttttggatttttagtagagacggggtttcaccatgttggtcaggatggtctcgatctgacttcgtgatccgcccgcctcggcctaccaaattgctgggattacaggcgtgagccactgcgccggccccacattaagtcttttaagtagcctgctgttaattctgtcgtagtcttttctataaaacaatactgatccaatttgtgataaactcacccactctcatcgcagaatatatatttctcccgttcatcttccttctttggcccactttatctgtctacctccttcattttgtttctcccaggaaggactgatgtaaatgggattttcagaaactccctccagcatgcccctctctgcatctggaatacaggggtcagaggatctactctgtctagggtccttcctctccctggcaggtcttatggtagtgtacactgtggtttgcctctatcagtctgtccttcatttctgtgaccagatctccaacaggacatgttctaaagaagtgtggtagaaactagcccatttcaaactttgcaccgtgatttttctacacgtcaccccactggggcttttgtgatcagttttataataaagagtggatctagattgaaccttagttttgctacagtgtttcattcagtcattccataaacatttattgagcgtgtactatgtgtcaggcagtgttctaggcatttgtaggaaagcgggagagagcagttggagcagtggacattgtgttcatttcagttgcagtcctccctgaagttttacagatctatagcaactcattttttttcagttcaaagaagcagtgggaaacctatgtctcctgctctcccccatgagatgtgtattgttttttcctgaatgagcagctggggacatagcccaggcaagtgaggagtcgtatgtaccaggaatgtccccctaggtcattactcatgggtattaggtgcccccaggagagaatggggaaatgatgcctcctgcctctcccagcatctgaggagggttaatgtaacagagtgttgaaagtcctttgcagacagaagcactatattgtgatagggccttaatattgtcttcgcagactgtcaagtcattcatgtcccaaggatctgggacagcttttatgcatatttcattttttctaaaacacttacctttcatataccttcagttttattcctgcatcttagaaagataactccagaatgactaaagaaaacatggtccaccaaggtggcatgtcagaaatgaccattcctagagttcccctgagaggctgattttcaaagaagttctccccaaagagcaacagaattgagatctatatcttaagggagatgtgaagtcctcttggcataatggccattattgccctggataaaaagcaaacattttagagacgccgaaagtacttagaacctaacagtcctctatcaattgcttaaagagtaaacagagaccttttataagaaacagacccagcgtcttggcagtttgcaaacaggatgaggaaatctttttttccttcctgaagcttcggatctgagaagctaaagacaccacatctgaattcacaaagtttcttttgtttaccataatattgtcctatctagatgagggtggtagctgcttgcctagggaaattttgagtatttggagccactgacactgtttctagacaaaagactcaagggttcctttgctggtcaacagtaaaagagcagaaaatcatctaacaggcacactggctcactcttgtaatcccagcactttgggaggccgaggcagacagatcacctgaggtcaggagaccaacctggccaacatggtaaaactctgtctctactaaaaaaatacaaaaacttagccaggcgtggtggcacatgcctataatcccagctactcaggatgggctggggcagaagaatcgcttgaacctgggaggtggaggttgcagtgagctgagattgtgccactgcactcctgcctgggcgacagagtgagaatctgtctcaattaaaaaaaaaaaaaaagaaaagaaaagaaagaaagaaagaaaatcaccaaacagcgagctccttggtggtgctcgtgttggagacgtaatttcaccatcctgaaaaatgacccaacaggcaacagcttggatggatcttaggggaatatgctgattgaaagaagctaatcactaaagcaatctaccatattattccatttatataacagtcctgagataacacaattgtagagatggagaacatattcatggttgccatgggttaggagttgtgggtggtggccataaaagagtggcgctagggagccttgtgatgatggaactcttctggctcttggctgtgttaactgatggtggttacatgaaactacacatggttatgctaagttacacacacacacacacacacacacacacacacatacacacacattagttattaaactgatggaatccaaataagctctgtggattgcattcatgtcaacttcctggttttgatattgtgatataatcatgccagctgtgaccattggaggaggctgggtgaagggtgcacaggacctcacataggacctcctgaacatttctttgtaacttcctgtgaatctataattattgcagaataaaggatttttttaattgcctttctaaactcataagtcttgagtgttgagattctaattctggaaaataatatatactatgagaggaatgaaaggaggaaggaatttatttccaattgaaagttttattttttatttttatttgtttattttttgagacagggtctcactctgttgcccaggctgagtgcagtggcatgatcacagctcactgcaacctcaacctctcaggctcaggtgatcctcccgcctcagcctcctgaggagctgggacgtaggcatgcaccaccacatctggctaattttttatttttttgtagagactagggcctaggctagtcttgaactcctgggcttaagtgatacctccacctcagcctctcaaagtgctaggattataggcgtgagccaccacacctggtctgaagaaggttttaacacctacacatacacacacacctaaccagatccaaccattcatcttactactgtctgaggcaggcattcctcctgctgtgtgtggcagagtacgtggaatgtgcctgcaggagcactcggagccctggctggcctgtcctcagggcactgctcctggggagtgaactcacttctgtatgtgcccagcttcgggagaaaagtgcctacaaccatctgcagaaaatctgtccagtttccagtgtgcctctctgggcttcttgaagcaaagcgaaacctaatttttaaattgaaaagcatgtctatacgtgttttgtaaagcattcaacgttacaagagagtgtgtcctaagctgtcactcagttgcttcccagagaacctttgtcaccaatgctatgtttaatctgacagaagcatgtgatggatggatgtgacatgtctacctttatagagatgttaacatcctatacaaacattttttgctcttactgttctttagagtagttctgtatcaacactattccattctttttttaacagagatagggtgtcctaaccagtgaatgtagtgatgggcatttgggtgatggtccttcttttgctctgtgtgtgtgtgtgtgtatgtgtgtgtgtgtctttgtctttgtgtgcctctttgagattagtcccttgtccaaaatatgttatttaaaataagtaagatctccttaacatttctgtttctctggccatttttatagttctgtgtgacttttccttctcgatatttgcctctggtgtgtgactcgtgatctccaagtaatccatcagcaccttaattttgagagcacagtgagagagcctttacgtcccttagggattctgaaggaattctccctcttgaggtctcagtccacaaatctatttttcttcataaaataatttgggatgactttctttgtagctttttataaaaagtggttgtgaaatgtagctattgtgttttgctgtctttacactgtactcattaacttcaacaaaattataaacgtaaaaatcatgtaaaagtatgtgtgataattacaggggagaataagatacccactgatgctctgatagttttttatacatgaatgtgtgtgctgtgtagatgttgctgtttcaaaatactgtcaatgaaattatttctctgtgtaaaggataaagcaaagtggcctctttcctattcacttccttggaattgccaaggctacatgaggcatagcagggatcggacgccgcagagtccctgactcttcccccctggtgtgtctatgagtgcaggggtgaggagatgaggtggaggcaggcctgaagatggccaccaggagaggactgccatccacagaagccatgatcactctggggtgccatggtgacatggtgacctcaccacctctcagtgggaagaggaggcttgtttcggttattctgcaggaaagtctgcaatagtagctctctcagaggagggggaaggaagggggtgggctgtgggccatagccgaccttgtcaggtaggcttgtgtgccagggccttgccacttcccagcctgtgtttagactgtgtgtcctgaaggccagctctccacctgacctctccccttggcattaccacataaaggaatcattgtattttccacgcaagtgttaggtattcttacttccctaaaatgagcaaggaggcgaaaacgctacataaaaaccagggtcctgagtttctgttcctcactgctgaacaggatggccccactcccatctatatggaaatgttggcaggggcccaggctactataaataagcatcctgactgctccaggaaggctgggcttcctcattttttttaaagtggaatttgtaacagcttctgcccagaacatttgtgtccaaggtcctttcatctcccaggcatcctgtacaactgccatacggaggttttggaaatcttcccgctgcagctgggtcacaggacaagccacacaggaccagctcactaaaagaagtgatgatctctcttctgtgacctccgtctcatattcagatgtgagttttaataggattccatctgaggaacaagcaggaatcttctttctccttctgccaaaaggcacatctgttagacacagaataggttcattagcaagcgcaaaagccccgggccacaggaaccatctgggcagcaggaatctgtctttcataccacatgatcctcagctcctaaggagtcttcacttccctcaggctgactggcaacacagggttacagcaagagtgccaggtagtaaaacatggaaagctacaacaaataaggctatggcactggcacaagaaccaagacaggtcaacagaacagaatggactgatggacagtgcaagtcagagcggtcggtggaactatcagttccagtccacaacaagataaggagcttgtgctagctcctaagtcagctgtgtcaccgaggacaccgctgagttaggctaccattttttttccctctcaatgaaggaggtgatgtgttgatttacattctggcccaagttatttaccttatggcaaacaggcactttgagtagctcagaagtgaacagtttttgaaacagtccctggtataatacttgaaagaaaaatcacaaaccaatagggaagggaagatatattcaacaaattgggtttggaaagttgaccatgttatttttttttaaataatgttttttcacagtaaacacaaaagaaattactaatagatttgaaagttaaatggagaagaaaatgaaatcatgaaaagaacctaaaagaaaaatcttaaactgatctcacaggacgaaaaaatgttaaatattttttcaaattaaaaaagtaataaaatatcccttgtagaaaatctgtaacatagtctttaaaaaagaaagagaaggctgggcacagtggcttatgcctgtaatcccagcactttgggaggctgaggcaggcggatcgcttgaggccaggagtttgagaccagcctggccaacatggtaaaaccctgtctctactaaaaatacaaaaattagccaggcgtggtggtgcacacctatagtctcagctactcaggaggctgaggcacaagaatcgcttgaacctgggaggcagaggttgcagtgagccaagatcttaccactgcactccagcctggatgacagagtgagactctgtctcaaaaaaagaaacaaagagaaattacccatacttctactccccagagatactactttgatttatttactaccaatgttattactattttattgcaactgatattattactatttccttccaatctgttcatataaaatttttattaaactagggtcctattatatatataattgtgtgtcattctttgccatgaaattaaatatcattttaaaacatgggaaaaaagaacatacaaaactgtatttacagcatgatcttgtggtggtggtggtggtgttttgtagagttgctttatatatacaaatagttattaaatacattaaattactatattctaaaataagtctaaaatgctatctggggttgggtgcggtggctcacgcctgtaatcccagtgcttcgggtgcatcacctgagatcaggagttcgagaccaacctggccaacatgatgaaaccccatctctactaaaaatacaaaaaattagccaggcatggtggcaggcgcctgtaaccccagctactcaggagggctgaggcaggagaatcgcttgaacctgggaggcagaggttgcagtgagccaagatcgcgccactgcactccagcctggatgacagagtgagactctgtctcaaaaaaaaaagttaaaaatgctatctggctgaacgtgacttttatttttatattttgggcctttccaaattttctgcaaggagtgtgtacactacatttgtcatcaaggaagataaaacaaaaagttgtcattcacaacccagtgttattggggctggttttggtggaattgactccctctgtcacaaactcagcttcagcccataccctgagccatagacctatccctctaatgcattgtactagtctcagggctaataacaagggagaggtgtcaaagggccagttccacctccaccaccagtggaaaagctattcccaggtgaggactgcagctgccagggcactgctccagaatgggcatgctggccatattcacttgcccacttctgcccagggatctatttttctgtggtgtgtattccctgtgcctttgggggcatctcttatactcatgaaatcaacagaggcttgcatgtatctatctgtctgtctatctatctatctatctatctatctatctatctatctatctatctatctatctatctatgagacagggtcttgctctgtcacccagattggactgcagtgggggaatcatagctcactacagcctcaaactcctgggctcaagcagtcctcctgcctcagcctcccaagtacctgggattataggcatgagccaccatgtccggctaatttttttttttaagagatggggtctcgctgtgttccccagccttgtcttaaactcctggcctcaagtgatcctcccatctcagccttccaaagtgctgagattacagcagaggcttttaagtcaaagctttccctgctaggacaagccctagttaaagtcctggagcactggccactgcagctgcacttggacagctggccctctgctacccttagtgccacctcttcttgggggctgcggcacgaagcactctgggaaacggggcttgagagggtttcattcctcagtgtttcctcgtgtttggcccaccctgagccaaggaaagctccttccattccactaaacggagggctcctctcccgagaggaagtttgcaggaaggttaggatccccctgcacgtggtgaggagtgtcctgccgttcactgcatcctggctccagtaattgtgagtaaaagaacaggttgggaggcattctcagaccatataatgcataggcaagataggtagcgaatgctgctcagtactttggaatcagctctcagggtagcacaaaggcattccttcaccagcagaacttcagagagatgacccattcaaatcagcagccttgtttttccaaatgcacactttagaatggcttatccatccatctttacagctctgcgtgccatttgtgaaaaacaaaagctacccaacaacctgggattataaaccaggccaaagtggtgagcacccagcagccatggtctggcttcccaccggctcctcacacagctccaagccaggctgtgttgggatggaagctttggtcctgctctcataagcctccctgttcttagagtgaccccacgcttctgtgcctcggcattgcaggcctggcgctggtgccgaggaagctctgtgcccactacacttgggatgccagtgtgctgctccaggcatggcctgctgtggacccggagttcctgcagcagcctgaggttgtccagatggcagttctggtaagtatctcccctcaaccccagaacccagcagtccctgccctgctggtggcccctaaatactgtggtggttttgagaggtctattagcaggcctttctagctcactcagctcagccctcagccacaccttgtgaccggaaggcacaaaggcctgtctttcctttgccagtaccctcagttttgtaggtcaccacacaccagaagcatgtgagcataaaactctcatggccctcaagtgcttggaaaggggagtaataaacctggcccgccttacaggttctcgttatcttcctttccaactgtggaccgtgtttcagtcactctcaatttcactcgtatgctcctggggacttttgggggctggggaagggtgacagtgaggtgggtggctatggagtaatacctgtaaaacatatttcagaaaggctccataatttttacttgaaaattaggtgattttaaatacataagaggagtttatcgtttaaaccctttctaagagcaagtaacccccttatagtttatcctatatgtgatttttctacatttgtttaatttgttaaatgagacacatttatggtgaatatctcttaaatatgctgatgtgtctgaagaaaaaaagaggacagggagtagacttggaaccacatgtctccccggaaccacacatggggccaggtaattcacattcaaaattgtcatttcactcttatcaccctataacgtgggcgttgccattctcattttttcagttgaggagactcagcctcagtgaagttggtccaccttgcccaggtttattcacaaatgcttagcaagtctgactctacatcaggcatagtgctctggggatacaaaatcaagataggacagacaggttccctgccctcatggagcttaaagcatagtggaagagatagggaagaataaaaaggaaagagtaagataaataaaatggctgcaaattgtgaaaagtgccctgaacaaacccacctaggggctgagggagggcataaccccacagaggttggccagccaggccctggcccctctgcacctgtgcagacccaaggcccagcaggaagctggaggtttggaaagacaatattttgcccaggagaccgggctccttcacagcccctttgtctctgcaccaactcccctggcactgtgttctctgccaccccaccccaagggaagaggatgtgagggtgccctggcaggcttcttccaaaaatcttcaaaaccaaaaccaaccatccttgtggcatggaggaaatttcttttctttctttattgttattattattaaaaccatccttgtggcatggaggaaatttctttttttatgattattattattatttaatagagacaaggcctcactctgttgcctgggctggtctcaagctcctgggctcaagcagtcctcttgcctcagcctcccaaattgctgggattacagacatgagccaccacacccaacccagaattttttttttttatttttttatttttttttttgagtgagacagagtctctctcactctgttgtccaggctggagtgcagtggcacaatcttggctcactacaacctccacttacagcaattctcctgcctcagcctcccaagtagctgagattacaggcatgtgctaccatgcccagctaatttttgtatttttagtagagatggagtttcaccatgttggccaggctggtttcaaactcctgacctcaagtgatctgcccacctcagcctcccaaagtgctgggattacaggcaggagccaccgcacccagccccagcccagaaatttatttaatttgagtaatgtttatgagaaataaatagctcatgcatccctgagaacttgtcctcagcccggttgagaaatgctaacttaagaaaaagagagggctttagtctatgaaaatatctatcttaaattaccactgcagaaaagaaattgagttttgatcgttggtaaaaataattttagataatcattgaaatgcaaacctaaaatgaaaagcatgtgtaagactccctgcaggtacacccagcttgagaacccaggctgggtcagcaccgtgccagcagcaaggacttctgtgaaggcctgggttctcgctccatgtgatctcactttgtgtcctggccagaaacaggctgcaagcgcagttcgatgtcattcagttcaattgtgcccatgttctttgtgcctatgacatgctgggctctgcccacaccagggatacagagacaagagctaccgccttgtcaccttggaacaaaggtctgggggcacatgagctgtcacacacagtttatgtaagggctcggaagtcaggccatcctgggtataagtctcagttttacaacatatttaacacctctaagcctcagtttcctgagttataaaatggaggcacacacagtgcctgccttatccggctattgagagaattaggtaggataatgccaggaagtgcttagcctgcttctggatgcactaatgacaaaatcctcttaatacagagtgagcgccgacccagggccacgctctgccctagaacttcccaggtgtgagcttatttactaagcacagtggccctgtgaggcaggcttgttgttatccccattcacagacaggcagatgtgtgactaaccctatcttacaggcaaggaaatggtggcaggatggagctaaataactcactgaaggtcacacagctagtccagaatatactctgaatggtgactgctgctagcatcagcgttgccacttctggcagttggagaatcagaaaatacgttatgaggaaaaggacaggcagctttggctttgaaagaggagcagcctctccccagtggagagaagggtgtcgcaataccaggaacaaacataggggggcaggcccacagagacccgggctgagccagggaggtactggagcagagggcatagggcagtgctggagacaaagctaaaggtagggagtgtcccctcctcagaggccagacacccctcagaggacaggggctaccagggtgtgtgagcaggagagagaggggctgtgtcaggtcatcccgtctctaccactgcctggggtggagcctaggtggacaagtgacttcacttcatgtgcctcagcttccctatctgtaaaatgggatgggatattcatgcatcgactcacaggttcatacaagtgaattgcatagttcaatgcttggcacagagtgagagctcagcaaatggaagcttgtactgttattagccatcgcaaggctaccctcatgatggggacaggtgatggattcgagtgggaggaacacctgtggcctgggccctgtccggcaggtgtcagaaaagtccaggagagtcatgcttaggccttctccaaagatggaggtgcaagagccccacagcatagcagcctctttttgaagtgatgtcaatgtgcttcttcctctttgggcccttgaagctcaggctcagtgttgagtcccaacctgacttctctgcccatctcattggcgctttatgccagagacagggggagcatgattggcacctactcaccctgaagggactgccttggtaaggcaactcaggggaggcctggggagccagaaccgtgtgagggtgcccgaggcagcaagggaaagactcagagcacgatggccacattcagagatcacatgccctgcccccaaagccccagctgtggacaagaatgactcaggtcccttggacaggctgtcaccagtgatgacttaggaccagcctgcccggctccagcaaagcacaggaagctcgaaggtttagaaatgggtcattgttgtgctgaggccatgcgtcaaagaggacatcccacagccagctccttctatcctggccagcactcttgacaaaggcagagggagtgcagcttagtgagctggtccctttacacaggttattcactccagaatctggaagaattctgtccttaaggaaaaaaaaaaaaaaaactaagctcaagatcccacagcttgtaagaccaggcctaagagtcaaacctgaatctccccacatgagagcaccatgctccccctcacccagcaccccctctgtaggtggtggcccagaggagatgttgccctgggctgctcttccataggaggaaagacatgattcactaataactgcatgtcctgagaattccctgttggggaccccaggacttcactaggggcatggctcttgggtgacagttgggaggtctcttccagggtctcagtccagccaagcatgtggccttgcaagaaggcagcttctaatggctcagaaacaccagggcaggggggccacagtccttcacaccacacacttagttccccagcttcagaacgctttaaaacgctgcagcatggagcctcacgctttcaaaacagtatttcataatatgccaggactttgtgttttattcgtcctctccctgtgacctaggctaatgcagacaataaaaatgccaccaagttctgcaaaactgaaaaaatgaagtgggtgattaggggacatttgataatgtgctttggttgaaacttgagaaattattcctagtaacttttcctaagactgtgactatattttccaagtgaaaaataagagctagtgaaagtaatgtgtgtgtagatttcggaacaatgggattgaggatttggaagcatgacttcagaaactgagccacgtggtcaccttccctcattgaatcaatattcggtgaatacccacctgtgtctggaactgtactgacccatgttaagcgagaaagagtgattaatgtgacaagatgtggctccaatttctaagtcctcacactagagcgcccccttctggagccagtcagcagagagagaagtttgactgtgtcccccttgaaaccgtcaactgtttggtccacttgagacctaggtaaaatctgaggggacagctcactctaacctggatctgaacttaaacttgagccccgatgacagaataatccctttctgcctggaaatggcccaagccatctcatgctgaggctttttgacataatctctaccacagctgtgtatgggagcagtgctcagaaaatgcaaggcggtcctcactccacgtacgggcactcacatgcagtctcagtgtcggtctctttctgcaggtctaggcattaatcctattcagctggttgcattttacccttatgggaagcagagtgctgacttcctcctttctccacctctattcctttcccctaaacaaatgcagccatgccctttacaggcaggaggtttggtattgggccgcctgccactctgaggatgttcctcattgtttatcttggcttttctccttctcagatcaacaataaagcttgtggcaaaattcctgtgccccaacaagttgcccgggaccaggacaaagtccacgaatttgttcttcaaagcgagctgggtgtcaggcttttgcaaggacgaagcatcaagaagtccttcctttccccgagaactgccctcatcaacttcctggtgcaagattgacagccaggaggctgcagctaccacgagggcctctgaggaacctccttccaggcctgggatgagggggcgatgtctgctggcccaggggaagggaaaagacaaatgtcttgactgttgacctcggtcctgtggcagactgcagtcaacagtgtgcctctgtagtgtggcctggtgctggggtgaaggtgagctgggcaaaggagaaatatgagctactgaggagggggttggacatcctgcccctcaccccccacccacactgcaggtagaggaggccatctgatcccatgggaagccatcagagacactgctggtgggagcaggaaggagcagtgcccctcgagcagccaggaagcctgcggatctgggaaatggctctgccttaggcgcttctcgggaatttgaggccagcctgaggaactgcaggactcaggtgcaatgtgccagccacttggaactgctaactgagcctccagatggtagtgaatggtctctttgccttcaggctggatgaggaagtcatttaggaaatgttcaaataaccaatatgtggaaatggacacagggatcttctgaagttgctttgaatcaaaaggcaggcagtgctggttcctctgcctgtgtccccaccactccccagctctgtcatgcaggcctgtcctccccaaccccagctggacgtgcctcccaggcctgctgtggttctgacacacaggatcccaggcaaggcaccacttcctcacatgaatgaggagcagcaagtcataaccactcccttgggtatacaatttgctgtgtagtgaagtggaaccaggctcaggctgctggtcccaacctcagagccccaccgcagcccagtagggatgcagcacgccccagagggctcatgtgggccccagatggcaatgccaccattgttgatgtgactccagagccagttattaggaagagcaagctcaccacagaggagtggaactgaggccccccagatgttgcctccggtgtccaagccacagcggtctggctgttgggaagatggccaggaatggactcataccattggcacattaggctaatcctggttttatgtgaagtcagcaattaagtgttcccactagaactgacctaagccactgattaatatttaatgagggaaggtaggggagaatctagccattttataatgccagaaatctatatatgttatctgatgccatttttctgaagtagcctcacatgtggtccccctgcagttcagcagttaacagatgacttttttagtgtaataaaatgtttatcatctatgacttcaggacatatgtgtgatcctggaatacaccatattccaatttcagagttaattacaaacagattgaggtattccatcatcatcggaagcattggctgtgtgtctgactccacatggaatgctataaagtgggatacatgtggttcttcttgttccagaacttacagtccatggtagacaacactgatacaggcaaactagcaacagcaattaagtcactggttctcagacttaatactctgcctggtcctcatgcccagatattccaatttaattggtccatggtgtggcttgaacaccaggaatctttaaaactccacaagtgattctaatgggcagcaaagtttgagaatcactggacacattgatgcacttgagccatcatttctatctagttcttcctcttcctaatcttcaggttagcggggataagtttggtccaggaccaagagttggaccaaaagggaagacaggggccagaagtctagagtgctagagccgacttgaatgtactcagtctcattgcacatggcagtgaatatggattaaatataagcccatgaaaatatttggtacaaggaaggagtgagtgattttgattatcttgttatttcctagtaagctaagtggtcttgaacaagatgagggaagggagccctttatttgcatgtgatctgaccagagcaatgggccagtaaagcctcaaggctgctagactgaccagctggctgacaccagcatggctctgaagaaattgctccaagaggataaagcttttgcaaacaagagctcatcagtacatactaagagacagtatgggtaatggaacaactggacagaaaactctgacccctgggagtgaggtggtggggaataatctcagccatttattgactgtcttgttgaggtcagaaactggactcagcaggttcacaataattatcttatttaattcccacaaaaactctagagggaaggaattattctctcataaaagaagaaacccaggctctgaggttcaataattgtctcagttttcacagctaataagtaggagaactgggatccaagcccacatcttctgtcctccacgtttgcctccccgccatgtctaccagtcccctcacctaattgtccatgttgcttggtgtacacagtcaggtgagatggctcccaaaaccactggcattttgagcagagtgtggctggggtttccctcctggaaagcctgtaaacctgggaaatcacagaggtggtgtcctctctgcaggggcagtggtgggtgagaggccaagagcatgtggttagttccagagagctgggaaaggtcatccagcaagtggaagacgtagactcgtgggcgggtatcagggttatttctattggggcaataagagaacagagtttgtgcccagggaaagtggtgctccatggcaaatgctaccacaataggagccagctgtctgcctaagaaaattgatctctaagaccctaaaacaattagtaagctgggaaacagcacaggcatatttcatcccagcctaaatgcacaccttcaggggattggctaagaaaagcagggtacatccagtcaatggaaggcgaggcagccagcaacagatggtttttcaaaaacatgtagcttggaaattttctcatgaatgaaggaagatacatgcacaatgcaatcctgaccattttttttcaacgcttacaaagaaaattgagagcaggaaatttgcttgcatatcataggctgagactcctacaagtcatttttttccttctgtctacttttttgcattttctcatttttattggccatgaagtatttcagtaatacttcatgaggcaaaacatcaaaatgtacaattttgtgaagctctaaatgagctaaaaacctgtttcccttgatttttcttttcttctcccaggcaggatatctagctgcctgtgaggatgtatcctagctaggaaaacatgctcctttccttgtatgaagtggaatccaaagaaacaaaaagcagcccccatgaagttcctgctcacggagttgctcagtctgggcaggtgtggggagctccctttcttgtatctcagtcttaggctcttgcaaccttgcaacagaagaacggaaggcaacgttcccctaatggaactctcctgctcacttgctgtcagattcaaggttgtggataagaccgaaaaaggaggcattatcacctccttttgcagataacgaaacttgagaccatgacagtaagtgatgatgtatcttgacccccaatctgtgctcattccacttagagcaccccctccccgtactaccatcaggcaaagtgtagtgcaggaggtaaactttctttctttcttttttttttttttttttttttgagacagcgtctcacttcatcacccaggctggagtgcagcggtgcagtctaagctcactgcaacctctgcctctggggttcaagtgattctcgtgccttagccacctgagtagttgggattacaggcatggaccaccacacccagctaatttaggaggtaaactttcttaactggcgggcccagctggactgtataagtcaaggaaaggcagaggggtagataaaaaggagagttttccctcgtctttcttcctgtgagaagagcaagcagaaaacagctgtttcgtgggctgggtccctgcctgacactcagagcgtgggtggctaaaggctctcatccaagctgatgagcttggagaaaggaatctagggtgaccaacaacgagcaagtagggaaatcccccgagggcgggtgggtggttggtgatggtgctggtagggaggggagagcctggagcagccagggaaaggccagtcctgtaggcagaggcctcagatccaagggtgggcccaatgacctcactccaggcctccagtgccctccctcagcctgtccacatgaggggctggaggcagctgtgcagagataatgaggccctggaggggaggtggtgaccaggactgctggaacccaatgccccatgactcatctccctgtgccttaagccctgaacttgaatttcagttcttattcagcctctggaagagcacacctaagacagacaccctgtatacaggggccatgggctgcaatctcccgacattacaacaataatatctatcatttctgaggactaattatgggccaggccctgttctaagtgctttgccacataaggagccttgcccaagaccacacacaagaaggtggcagagccgtgatcccactctgctccacctcgctggttcccaaagacctttatgcaaaccagcccttgaacctcctgaattttcccagatggtccttatttatagctttctctccagttgttccccaaatgccctagagcctgccaaaaccacacttcctcatgtgtatctgtgaacgttggctcgctgtgggcacacacctggttgaatgcatccctgaagttctgaggcacccaattcagcatgtggggtcggctctctctcagcctgggactgacggctcccagggagccatcacccctggcagacagagcctcctttggacaccatccctgggacctagaagccaggaagccatccttgctcaactccacctactgtgaggaaaatcccatggcagaggggagaggggagacctgttctagacctccccgatgataagcactccttggcatctcagcagccccatctgtgtcttccgcagatcatcttttccacaaacaatagaagagccccatctgggattaattgaggagggttcagccctggtcccatccaagggaggcctcacctaccacatctgctgctgcatactcatggctgccttcagaactcagaggagaactagatcttaggggaggggttccccttcctcaggcactgtcgcgggggtgtagaaaggaataatgtgtcctttacactgtgagatccatagaggaagcaggcacccaaagattgatgccattcattgggtctggtttgtcaatcatcaaggccagactgggccagaggggctttgtcaggtggactcagagagagactcacttgtcagtgcacaagagggcagccactgcacagacataggctgagagctgcgaagagggaacctaaagcaagtgtcgccagatatccccattattagttatcagctaactcttagttttcagttaacatcatagtttccaggaaatattttagttttctattaatattttaattaactactagcatcagagtcatcagcaaatatctaagtcatcagtaaacatcttaggtgtcagtttcctctgagtgaatgtagtagacctcttagtcatcatttacttcattattaccagttatgcactaataattagtggtctgctaatatctcgtctaccaatcatcttcagcaataaacatgctttaattatcagctgataattaatgtagccataacaaataaacccccaaatgtcagtggtctgacccagcaaactttatttctctttcccatgagtctaatcctctagagaagggaacaggggaggaggttctgttccgttcagttccagcttgacggagggttcgtgcagactacacagtcttcagctcttggctttcatggttgcccctgacatcaaatccagcccgcagataagggggagagaggtggagtgtcacagtggctttggccaggtctggaaacagtgtacatcacttctgcccacttcctcttggccagaattcagtcacgtgcctcagccaattgctaggggctgggtagccacttcccagcagcaactctgcaggagggaaggggggcctgatcttggagggcagctggctgcctctgccacatggacaatctttgttttcgaattgttgcttgtcagtgtacacctaagttaccttctaagtatctcaggtgtctgctaaccaccttgttttgcagctactcctgagtcacagctgtttggcttctaaatctgtgacagctgctgtgagtctaaaatagcagagcaatcacagactcttgttgcctgccaaactgggatggtgcctgccccttgctcgagactttgcatggagatgtgtgaagtcacagacgtgctttgtgccattttataaaataccgggtttactccgagggtttgtttattggtccccaattctccccaccttccttcttctgtgaggctaacacattggtttgtgagacagtttggagtattttgttttagggggtgtcaggacattttgaaaggtagagaaagtcaggcttcccagatgccctgcaggaaacctgggaaattctccagattcctgctcctaaaaggtcatggctgctcattcagggtggaaatgtgacacttccccagaagcctcactgtgctctggtctaggagactcacacctcaccccaacacacacacacaccccaccccaccccccccacacactacatacaccatacaccacctacaccacaccacacacatgcacacgtatacaccacataccacacattcatacacaccacacagctgagggcctgctttgggggaaggtaatttatttttgcctgacagaagaacgtgcctcacaccatgtggctgtggaggtggggagggcaacgtagtcttgagagttcatcagagtcacagctgttagggaagtgcctgggcctggggccaaggtggacagtgaaacaggaaggctgcccccatttccacatgtgctcacatggggctcccccacctgaggcctccaactacaggtgcacccctcccctgacagcacccacctctggtgggagtcttctgtatgattaataaaaagcgtagtaataataatcatatgtaatggccatggtatattgagtgttactttttttttttttttagacaaggtctcactctgtcacccaggctggagtacagtgtcatgacactgcagcctcaacttgccaggctcaagtcatcctcctgcctcagcctccctaatagctgggaccgtaggcatgcacaagcacacccagctaattttttttgattgtttgtacagacagggacctactccctttgtgccacagattgagcttgacagatggcccagagggtgagggggctttcaggaacttgtctgggctctattgtttatgaaccatgagatctttgcaggctccttaacctttctgtgcctctgtttcctcctctgtgaaatggagagaatggctccaacctctgccttgtgagggtgcactgaagcaatgtgtgtgtaacaggctccggacagcgcagggcgcacagtgagtgcactactcttcggtcatccagcactcttaggaggacgtgtttcatcccatggaatcctcaccagccatgtgtgaggagtgctcactttacagatgaggaaacaggcttggcaatatcaggtaacctgcctgaggccacctggcctcccttctgctccggcagaagccccaccctggcctgggtaagcctgggagccagtgccacatcctgatggggatgtggacagggaacaggcagcctctccgccctccagtgccctgaggagcagcctccagaggaagaggagttgccagtctccctggcctgccaggaaaaggtgagtcctgaggcagtcctgtggggtgcattggctatccagggagggagagaggaggaggtcagaggcctggtcttgattctcatgggcttcccttcctgcagcccttccttttcctttggccactcatgactgcgggttccagcctgggcaacatagtcagaccccatctctacaaaaatttaaaaattagccaggtgtggtgtgcacacccgtgtccccagctactcaggaagttgaggtgagaggatcacttgatcccaggaggtgaaggctgtagtgagctaagatcacgccactgcattccagcctgggtgacagagcgagaccctatctcaataaaaattttaaagactactgagtgaccacaggtggggtgggtgttccctgggggctgcagcggagatcaggctcaatcgaggtccatgctgggacccgtgagaggtagtgacaaacaagggcctcccagagggtagttgaggctcaaggttggctttgggagtcagggagagacaatggtagtgggatggaggggtcttccatcttgttttttttttctaggtgctgatctctccctgaaacccaagggtcagttcaagagataggaattgtgactgtctgttgctcccccagggtgtgtgtgtgcctgtgcccaggcactgagcagtggctcaagggcagagtccaggggaggacaaagaccaccaggtcacctagagggttgtgctcacctgcacctgctgccacaaccccacaagctactacaaaccttggatcagcctcactgctctgtagtatgctaagcaaattatatctggagttgaataagacagtctgtccattcccccacctcactcctcactgcttcttggataggcagggtgcgctcctgcctcaaaacctcacacctgctgtcgctctacccagagagctcctccccacatggctcactctctcctccttctgttgtctgctcccagtgaggtcttccctggccatcctacataaaataccaccccaccccagcactccctaccccctagcaaatagcagtatattttgcttaatttcctgtgtcttgttctatcatttgtcttcccttccagaatatcggcatcatgagggcagggacttttgcagctttatttatttatagcagttttatcatctattatatctccagacctagactgccattcaggcacaccataggtgctcaataagtatttgaacgaatgaatgaacccctgtgcacctggctagtgtattatgggagtcagacaactcactgcagcccctagatactcagggggatcccaggccggcaccatgggcatcaccttgggtgcttgttagaaatgcttattcttggaccccaccccagacctgttgaatcagaaactgcattttactgagatcattggatgattcccaaatgcacattacagtctgagaagagctacactcattctggaaaatggtccctctggggccttccctagaatgcccatctccagtatggctcacaaaaccctcatgaacaatgtggcccctgcccatctcatgaacctgttctccaggccctggaccaccacacattctccactccagacacaggaggcccaagcaggtgtcagggaccagcactccatctcccctccgggccttcgcaggggctggtctctcccagaacacccttccccgctctccctggctagctccttgtgtgccttgaacaatcactgaaagggtttaggctacagagaggcaaggtcagaacctttttttaaaaaaaaacaaaaacaaaaacagagtctccctcttgtcacccaggctggagtgcaatggtatgatcttggctcactgcaacctccacctcccgggttcaagcgattctcctgcctcagcctcccaagtagctgggattataggcgcctgctaccacccccggctaagtttttgtattttcagtagagatggggtttcgccatgttggccaggctccaggctggtttcgaactcctgacctcaggtcgtccgcccacctcagcctcccaaagtgttgggattacaggcgtgagccactgcgcccagccagtgcccagagcaaggtggtgaggagctcatctggggccagcccccgagcagacagggaatggctggggctctgggtgagggcagagctgtggaatgagagagaaggggcagatggagcagtgttgccagtggacaggccctggcagcatgctggagagggaaagaccaggactggaacatcagcacattactagggtagagggtgctgccttgtggggacagccaggagggcaggctcattcaggggtccagagcctacagctgagggggcctggctgggaaggggtcactgtgtgcccttgagccaccacgcgtgaacctgctttgtatcctcccgaggaaattgagttaaactcctaagatacttaataccttgagttaatctcctaagacaggatctcaaggtagggcagactgagaatgtgtccattccaaggaggcatgtccctggggccagcctttgggttatgatggctgaagtgagtcagcagcgtgtgtttcgccctgcatggcccatggagaggcctctgggaggcaagtgcccatttgcttccctgatggctacctctctgcccatgtgcagaaggtggtgagaaggcaggagaagaaaggccacagctctatatctctttgttgtaaccatcacaaggacatgggactcgggctgcagaggccctcagaggtcttctagcccagggatgctcagctgcctccccagtccctctttcagggaacctgctcacctcatgagagacaaaccaccatttttggaggagtttgaatacagtttctcagcatcttgttgcaatcgctatgcacagggaggtttaagactctacagctggcttctcaattgttgccagggagcctgcagaaccaacctgccctccccacagctggataacctgtctcaccagtgcacagttctccagtttagcatctctgaagtcaagatgtgttcttaaagggaacagttgtgatgtcattgctgcaaatcttgcacatgaccactggtaacttggaagaaaatcccagagacaagagtggagcagcttgttaggaaatgccacatcctcagtgctcgtgacgatgggaaggatcagagcatagaagatgtggacatcacaactcttgtaaaacagtgattcagaagagttctgaatgaacaaatttcagccatgccttaactaacttattttacttgtattttcctttttatatatgtatcatagtgatatatggtaaaatttatctaaataatgcccgggcacggtggctcatgcctgtaatcccagcactttgggaggcagagatgggcagatcactttgaggtcaggagtttgagaccagcctggccaacagggcgaaaccccgtctctattaaaaatacaaacattagccaggcatggtggcacgtacctataatcccagctacttgggaggaggctcttcgaagcatgagaatctctggaacctgggaggcagaggttgcagtgagtggagattgcgccactgcactccaacctgggtgacagagtgagactttgtctaaaaataataataatctaaatttaaaaataataatctaaataagtctgaagtatgtagttcaataagtatagaattaaaaatctaagtaataagagaacaccgtgccatagtttaatgtcagcatttttttgttgtgtgtgtgtatgtgtggtacatgcaccttaaaatcagtgagatctttgatttaatgaaatgtggataattgtccattgaagtctacttaataccttgaggtagactcctaagatagcattgtactgaaatcgtcttggtgtctgttttccctgctaggctggctacttccttgagggctgagcagcaccttgatgatggagagtctctagaatctaacacaatgtacagtaggggagcacagtaatgtcctgtcaaccactgggtttgggtagtggtgtattcctatataccagccataaaggtacctgcacttttttttttctgggagggtgggcagagtttagaatccctaaaccacttcatcagcaaggcccctggtgtggttgtatagattgtgcactgcacagaggcacccagacaagagacccagtggggctgaagttggtggctgccctgctgcccaagccacgtgccgcccaggccatgtgcccgtgggactgactgcccagtacaggcagcttttctcatttttacaaacatgcgatggaggtgatcagtggatttgttcctcaggccctaagtggaattttccctgcagtgcctctgaccttaggagtcctaagcagctggtcaggatgggctaaaagcctgaccttcagattggcccagatgaatgttgttggggcgggggaagtggttacagacttttgggtgaagctggcggtgcaggaaaaactttagttctgattctgaccctacgccggctctgtgacctcaagttagtgcaacagaaatgggattttgatttctttaattccaaagttgactcgggggtgccagctggtgggtagggtgggagttctgagaggaaacagtgctgaaaatagtttggggacattctgtaagttgaaaactgcagagcagccctaaatgatgcctggagatttctcctcatggggaacaaaagggtaacaattggagggtttcctgcttggcccttcagttaagactttggctctggtctctctggctgccacagaatcactgattgttgaggatcagccgtggggcatcgtctgaacacagtagcccattgcaaaatagtttgcgtgtgttttcaagtcttggtggccaagagctgggctgaccccaaaagcgaagaagccggacggggaagcaaagacagctgcgaggagggagtgagggagctgccagaggaagtactgtgggtctattttaagactggaaaattggaaagctgaggaagtacaacgtgtttcagtaacggggcccagcaatccctggccttcgcggggatatttcccctgaggtgagagtacagaaggactgaacatttctattcaccaaaatgctgaggccagagctcatgggaaccaggaaggggagaggagtggggaggacaaccaagtgtttaaactagatagaagccactggatgtgttggtggttctggaccttggctatggatcagaatggccctctctgggccagaactgaaaaccactgggcagtggaataatggagagattggcggatgatggcctgtgggccaaagctggcctagcatctgtttttgtaaatgaagttgtactggaacactgccatgcttaattgtttacatatcatctgtggctgtttttgaacagagttgagtagttgcgacagagactttatgtcctacaaagtctaaaatatttattatctacccctgtacagggaaaatctgtcaatgacacaagagtcactgggcttgggggtctaaaaacttgggttgagttctagtgtacctatatccctatctggtgacctagggcaaagctttagccaagcctcagtttctttctctgtaaatcaagtagcaatacccatcttacccaggtgtgaatattgaacagataatatatttattgtactcctcggaggtatttttagttattattttagatattacaacatacaaccttataataatcttccttgagttaatattataccactttacaaacaaggtaaaaattgaaccattcttgtccgggcgcgatggctcacacctgtaatcccagcactttgggaggccgaggtgggcagatcacgagatcaggaaatcgagatcatcctggctgacacagtgaaaccccgtctctactaaaaatacaaaaattagccaggcgtggtggcaggtgcctgtagtcccagctactcgggaggctgaggcaggagaatggcatgaacccgggaggcggaacttgcagtgagccgagatcacaccactgcactctagcctgggcgacagagtgagactccatctcaaaaaaaaaaaaaaaaaaaaaaaattgacccattcttgcaacagtttcccttccctgtcctttgaaggtatgggcatatactttttctacatattttataaatcccacataaatgcatattatttttgcttcagacagtcaagtctttaaagaaattttaaaaagaaaaaatacttttatatttacctcaatagttaccatttctggagttctttcttcttcctgtagttctgggcttcaatctgagataaattccctttagcttaaagcattttctttagtgtttcttatagtggttctgctggcagtaagttctcatagcgttggcttgagaaacctttctttccctataccatactggatatagccttctagcatggtacttttttttttcaacattttagatgtcattccattgactcctggatttcattgtttcagatgaaaattaaccatcattctttttgttgttgtattaaagataatgttttctttcctt

SEQ ID NO: 7 - DNA, 8224 bp, Homo sapiens
gctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggccaagatgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacgtcgaatgagtgatttgagtgtaattggtcatccaatagattcagaatctaaagaagatgaaccttgtagtgaagaaacagatccagtgcatgatctaatggctgaaattttacctgaattccctgacataattgaaatagacctataccacagtgaagaaaatgaagaagaagaagaagagtgtgcaaatgctactgatgtgacaaccaccccatctgtgcagtacataaatgggaagcatctcgttaccactgtgcccaaggacccagaagctgcagaagctaggcgtggccagtttgaaagtgttgcaccttctcagaatttctcggacagctctgaaagtgatactcatccatttgtaatagccaaaacggaattgtctactgctgtgcaacctaatgaatctacagaaacaactgagtctcttgaagttacatggaagcctgagacttaccctgaaacatcagaacatttttcaggtggtgagcctgatgttttccccacagtcccattccatgaggaatttgaaagtggaacagccaaaaaaggggcagaatcagtcacagagagagatactgaagttggtcatcaggcacatgaacatactgaacctgtatctctgtttcctgaagagtcttcaggagagattgccattgaccaagaatctcagaaaatagcctttgcaagggctacagaagtaacatttggtgaagaggtagaaaaaagtacttctgtcacatacactcccactatagttccaagttctgcatcagcatatgtttcagaggaagaagcagttaccctaataggaaatccttggccagatgacctgttgtctaccaaagaaagctgggtagaagcaactcctagacaagttgtagagctctcagggagttcttcgattccaattacagaaggctctggagaagcagaagaagatgaagatacaatgttcaccatggtaactgatttatcacagagaaatactactgatacactcattactttagacactagcaggataatcacagaaagcttttttgaggttcctgcaaccaccatttatccagtttctgaacaaccttctgcaaaagtggtgcctaccaagtttgtaagtgaaacagacacttctgagtggatttccagtaccactgttgaggaaaagaaaaggaaggaggaggagggaactacaggtacggcttctacatttgaggtatattcatctacacagagatcggatcaattaattttaccctttgaattagaaagtccaaatgtagctacatctagtgattcaggtaccaggaaaagttttatgtccttgacaacaccaacacagtctgaaagggaaatgacagattctactcctgtctttacagaaacaaatacattagaaaatttgggggcacagaccactgagcacagcagtatccatcaacctggggttcaggaagggctgaccactctcccacgtagtcctgcctctgtctttatggagcagggctctggagaagctgctgccgacccagaaaccaccactgtttcttcattttcattaaacgtagagtatgcaattcaagccgaaaaggaagtagctggcactttgtctccgcatgtggaaactacattctccactgagccaacaggactggttttgagtacagtaatggacagagtagttgctgaaaatataacccaaacatccagggaaatagtgatttcagagcgattaggagaaccaaattatggggcagaaataaggggcttttccacaggttttcctttggaggaagatttcagtggtgactttagagaatactcaacagtgtctcatcccatagcaaaagaagaaacggtaatgatggaaggctctggagatgcagcatttagggacacccagacttcaccatctacagtacctacttcagttcacatcagtcacatatctgactcagaaggacccagtagcaccatggtcagcacttcagccttcccctgggaagagtttacatcctcagctgagggctcaggtgagcaactggtcacagtcagcagctctgttgttccagtgcttcccagtgctgtgcaaaagttttctggtacagcttcctccattatcgacgaaggattgggagaagtgggtactgtcaatgaaattgatagaagatccaccattttaccaacagcagaagtggaaggtacgaaagctccagtagagaaggaggaagtaaaggtcagtggcacagtttcaacaaactttccccaaactatagagccagccaaattatggtctaggcaagaagtcaaccctgtaagacaagaaattgaaagtgaaacaacatcagaggaacaaattcaagaagaaaagtcatttgaatcccctcaaaactctcctgcaacagaacaaacaatctttgattcacagacatttactgaaactgaactcaaaaccacagattattctgtactaacaacaaagaaaacttacagtgatgataaagaaatgaaggaggaagacacttctttagttaacatgtctactccagatccagatgcaaatggcttggaatcttacacaactctccctgaagctactgaaaagtcacattttttcttagctactgcattagtaactgaatctataccagctgaacatgtagtcacagattcaccaatcaaaaaggaagaaagtacaaaacattttccgaaaggcatgagaccaacaattcaagagtcagatactgagctcttattctctggactgggatcaggagaagaagttttacctactctaccaacagagtcagtgaattttactgaagtggaacaaatcaataacacattatatccccacacttctcaagtggaaagtacctcaagtgacaaaattgaagactttaacagaatggaaaatgtggcaaaagaagttggaccactcgtatctcaaacagacatctttgaaggtagtgggtcagtaaccagcacaacattaatagaaattttaagtgacactggagcagaaggacccacggtggcacctctccctttctccacggacatcggacatcctcaaaatcagactgtcaggtgggcagaagaaatccagactagtagaccacaaaccataactgaacaagactctaacaagaattcttcaacagcagaaattaacgaaacaacaacctcatctactgattttctggctagagcttatggttttgaaatggccaaagaatttgttacatcagcaccaaaaccatctgacttgtattatgaaccttctggagaaggatctggagaagtggatattgttgattcatttcacacttctgcaactactcaggcaaccagacaagaaagcagcaccacatttgtttctgatgggtccctggaaaaacatcctgaggtgccaagcgctaaagctgttactgctgatggattcccaacagtttcagtgatgctgcctcttcattcagagcagaacaaaagctcccctgatccaactagcacactgtcaaatacagtgtcatatgagaggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaaatactatcatagatattgatcatactaaacctgtgtatgaagacattcttggaatgcaaacagatatagatacagaggtaccatcagaaccacatgacagtaatgatgaaagtaatgatgacagcactcaagttcaagagatctatgaggcagctgtcaacctttctttaactgaggaaacatttgagggctctgctgatgttctggctagctacactcaggcaacacatgatgaatcaatgacttatgaagatagaagccaactagatcacatgggctttcacttcacaactgggatccctgctcctagcacagaaacagaattagacgttttacttcccacggcaacatccctgccaattcctcgtaagtctgccacagttattccagagattgaaggaataaaagctgaagcaaaagccctggatgacatgtttgaatcaagcactttgtctgatggtcaagctattgcagaccaaagtgaaataataccaacattgggccaatttgaaaggactcaggaggagtatgaagacaaaaaacatgctggtccttcttttcagccagaattctcttcaggagctgaggaggcattagtagaccatactccctatctaagtattgctactacccaccttatggatcagagtgtaacagaggtgcctgatgtgatggaaggatccaatcccccatattacactgatacaacattagcagtttcaacatttgcgaagttgtcttctcagacaccatcatctcccctcactatctactcaggcagtgaagcctctggacacacagagatcccccagcccagtgctctgccaggaatagacgtcggctcatctgtaatgtccccacaggattcttttaaggaaattcatgtaaatattgaagcaactttcaaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaaccgatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatcagaacagcaagtggcagcgagaattcttgattccaatgatcaggcaacagtaaaccctgtggaatttaatactgaggttgcaacaccaccattttcccttctggagacttctaatgaaacagatttcctgattggcattaatgaagagtcagtggaaggcacggcaatctatttaccaggacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagttgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcgaacatgtgttttcatcatttcagccaaagtcctaacttcctgtgcctttcctatcacctcgagaagtaattatcagttggtttggatttttggaccaccgttcagtcattttgggttgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccagg

SEQ ID NO: 8 - DNA, 3777 bp, Homo sapiens
tggctgagtggctactctcggcttcctggcaacgccgagcgaaagctatgactgcggccgcgggttcggcgggccgcgccgcggtgcccttgctgctgtgtgcgctgctggcgcccggcggcgcgtacgtgctcgacgactccgacgggctgggccgggagttcgacggcatcggcgcggtcagcggcggcggggcaacctcccgacttctagtaaattacccagagccctatcgttctcagatattggattatctctttaagccgaattttggtgcctctttgcatattttaaaagtggaaataggtggtgatgggcagacaacagacggcactgagccctcccacatgcattatgcactagatgagaattatttccgaggatacgagtggtggttgatgaaagaagctaagaagaggaatcccaatattacactcattgggttgccatggtcattccctggatggctgggaaaaggtttcgactggccttatgtcaatcttcagctgactgcctattatgtcgtgacctggattgtgggcgccaagcgttaccatgatttggacattgattatattggaatttggaatgagaggtcatataatgccaattatattaagatattaagaaaaatgctgaattatcaaggtctccagcgagtgaaaatcatagcaagtgataatctctgggagtccatctctgcatccatgctccttgatgccgaacttttcaaggtggttgatgttataggggctcattatcctggaacccattcagcaaaagatgcaaagttgactgggaagaagctttggtcttctgaagactttagcactttaaatagtgacatgggtgcaggctgctggggtcgcattttaaatcagaattatatcaatggctatatgacttccacaatcgcatggaatttagtggctagttactatgaacagttgccttatgggagatgcgggttgatgacggcccaagagccatggagtgggcactacgtggtagaatctcctgtctgggtatcagctcataccactcagtttactcaacctggctggtattacctgaagacagttggccatttagagaaaggaggaagctacgtagctctgactgatggcttagggaacctcaccatcatcattgaaaccatgagtcataaacattctaagtgcatacggccatttcttccttatttcaatgtgtcacaacaatttgccacctttgttcttaagggatcttttagtgaaataccagagctacaggtatggtataccaaacttggaaaaacatccgaaagatttctttttaagcagctggattctctatggctccttgacagtgatggcagtttcacactgagcctgcatgaagatgagctgttcacactcaccactctcaccactggtcgcaaaggcagctacccgcttcctccaaaatcccagcccttcccaagtacctataaggatgatttcaatgttgattacccattttttagtgaagctccaaactttgctgatcaaactggtgtatttgaatattttacaaatattgaagaccctggcgagcatcacttcacgctacgccaagttctcaaccagagacccattacgtgggctgccgatgcatccaacacaatcagtattataggagactacaactggaccaatctgactataaagtgtgatgtttacatagagacccctgacacaggaggtgtgttcattgcaggaagagtaaataaaggtggtattttgattagaagtgccagaggaattttcttctggatttttgcaaatggatcttacagggttacaggtgatttagctggatggattatatatgctttaggacgtgttgaagttacagcaaaaaaatggtatacactcacgttaactattaagggtcatttcgcctctggcatgctgaatgacaagtctctgtggacagacatccctgtgaattttccaaagaatggctgggctgcaattggaactcactcctttgaatttgcacagtttgacaactttcttgtggaagccacacgctaatacttaacagggcatcatagaatactctggattttcttcccttctttttggttttggttcagagccaattcttgtttcattggaacagtatatgaggcttttgagactaaaaataatgaagagtaaaaggggagagaaatttatttttaatttaccctgtggaagattttattagaattaattccaaggggaaaactggtgaatctttaacattacctggtgtgttccctaacattcaaactgtgcattggccatacccttaggagtggtttgagtagtacagacctcgaagccttgctgctaacacctgaggtagctctcttcatcttatttgcgagcggtctctgtagagtggcagtaacttgatcatcactgagatgtattgtatgcatgctgaccgtgtgtccaagtgagccagtgtctgtcatcacaagatgatgctgccataatagaaagctgaagaacactagaagtagcttcttgaaaaccacttcaacctgttatgctttatgctctaaaaagtatttttttattttcctttttaagatgatacttttgaaatgcaggatatggatgagtgggatgattttaaaaacgcctgtttaataaactacctctaacactatttctgcggtaatagatattagcagattaattgggttatttgcattatttaatttttttgattccaaggttttggtcttgtaaccactatcactctctgtgaacgtttttccaggtggctggaagaaggaagaaaacctgatatagccaatgctgttgtagtcgtttcctcagcctcatctcactgtgctgtggtctgtcctcacatgtgcactggtaacagactcacacagctgatgaatgcttttctctccttatgtgtggaaggaggggagcacttagacatttgctaactcccagagttggatcatctcctaagatgtacttactttttaaagtccaaatatgtttatatttaaatatacgtgagcatgttcatcatgttgtatgatttatactaagcattaatgtggctctatgtagcaaatcagttattcatgtaggtaaagtaaatctagaattatttataagaattactcattgaactaattctactatttaggaatttgtaagagtctaacataggcttagctacagtgaagttttgcattgcttttgaagacaagaaaagtgctagaataaataagattacagagaaaattttttgttaaaaccaagtgatttccagctgatgtatctaatattttttaaaacaaacattatagaggtgtaatttatttacaataaaatgttcctactttaaatatacaattcagtgagttttgataaattgatatacccatgtaaccaacactccagtcaagcttcagaatatttccatcaccccagaaggttctcttgtatacctgctcagtcagttcctttcactcccaattgttggcagccattgataggaattctatcactataggttagttttctttgttccagaacatcatgaaagcggcgtcatgtactgtgtattcttatgaatggtttctttccatcagcataatgctttgagattggtccatgttgtgtgattcagtggtttgttccttcttatttctgaaaagttttccattgtatgaatataccacaatttgtttcctccccaccagtttctgatactacaattaaaactgtctacatttac

SEQ ID NO: 9 - DNA, 12227 bp, Homo sapiens
tgagctgcgaggcccgagagtcagaacctgggggagagggatggtctctgcacgggggggagccggaggagccgccgccgctgccgacgccaccgccgcagccgccgccgccgccgccccggcacccgcctcccggcgctgacggtctcgtacgaagccggcgagggggagccagcagcggcggtcgccggcacgccgcccagcatggtccgggaaaccaggcatctctgggtgggcaacttacccgagaacgtgcgggaagagaagatcatcgagcatttcaaacgatatggccgcgtggaaagtgtcaaaattcttcccaagaggggatctgaaggaggagtggctgcctttgtggattttgtggacatcaaaagtgcacagaaagctcacaactcggtcaacaaaatgggtgacagagacctacgcacggattataatgaaccaggcaccatcccgagtgctgctcggggattggatgatacagtttccatagcatctcgtagtagagaggtttctgggttcagaggaggtggtggagggcctgcttatggtcccccaccgtcacttcatgcacgagaaggacgttatgagcggagacttgatggggcttcagataacagggagcgtgcttatgaacatagtgcctatggacaccatgaacgggggacgggaggatttgatcggacaagacattacgatcaggattactatagagatcctcgagagcggactttacaacatgggctctattacgcttctcggagtcgaagtccaaatcgctttgatgctcatgacccccgatatgaacctagggctcgcgagcagtttacactgcccagtgtggtacacagggatatctacagggatgatattacccgggaggtacgaggcagaaggccagagcggaattaccagcacagcaggagtcggtcaccacattcatcccagtctagaaatcagtctcctcagagactggctagccaagcatctagacccacaaggtcccctagcggcagcggctctagaagtagatcctccagtagtgattcaatcagcagcagcagtagtaccagcagtgacagcagtgattccagcagtagttcaagtgatgattctccagctcgatcagttcagtctgcagcagtccctgcacccacttcccagttgctttcatctctggaaaaagatgagccccgtaaaagttttggcatcaaggtccagaatcttccagtacgctctacagatacaagccttaaagatggccttttccatgaatttaagaaatttggaaaagtaacttcagtgcagatacatggaacttcagaagagaggtatggtctggtattctttcggcagcaagaggaccaagaaaaagccttgactgcatcaaaaggaaaacttttctttggcatgcagattgaagtaacagcatggataggtccagaaacagaaagtgaaaatgaatttcgccccttggatgaaaggatagatgaatttcaccccaaagcaacaagaactctctttattggcaaccttgaaaaaaccactacttaccatgaccttcgcaacatcttccagcgctttggagaaattgtggatattgacattaagaaagtaaatggagttcctcagtatgcgtttctgcaatactgtgatattgctagcgtttgtaaagctattaagaagatggatggggaatatcttggaaataatcgcctcaagctgggttttggaaagagcatgcctacaaactgcgtgtggctagatgggctttcttcgaatgtgtcagatcagtatttaacacgacatttctgccgatatgggcctgtggtaaaggtggtgtttgaccgcttaaaaggcatggccctggttctctacaatgaaattgaatatgcacaagcagctgtaaaagagaccaaagggaggaaaatcggtgggaataaaattaaggtggattttgcaaatcgggaaagtcagctggctttttatcactgcatggagaaatctggtcaagacatcagagacttttatgaaatgttagccgaaagaagagaggaacgaagggcatcctacgactataaccaagatcgtacatattatgagagtgttcgaactccaggcacttatcctgaggattccaggcgggactatccagctcgagggagagagttttattcagaatgggaaacttaccaaggagactactatgaatcacgatactacgatgatcctcgggaatacagggattacaggaatgatccttatgaacaagatattagggaatatagttacaggcaaagggaacgagaaagagaacgtgaaagatttgagtctgaccgggacagagaccatgagaggaggccgattgaacgaagtcaaagtcctgttcacttgcgacgtccacagagtcctggagcgtctccctctcaggcagagaggttgccgagtgattctgagaggaggctttacagccgatcctcagaccggagtggaagctgtagctcactctcccctccaagatatgagaaactggacaagtctcgtttggagcgctatacaaaaaatgaaaagacagataaagaacgaacttttgatccggagagagtggagagagagagacgcttaatacggaaggaaaaagtggaaaaggacaaaactgacaagcagaaacgcaaaggaaaggttcactcccctagttctcagtcttcagaaacggaccaagaaaatgagcgagagcaaagccctgaaaagcccaggagttgtaataaactgagcagagagaaagctgacaaagagggaatagcgaaaaaccgcctggaactcatgccttgcgtggttttgactcgagtgaaagagaaagagggaaaggtcattgaccacactcctgtggaaaagttgaaagccaagcttgataatgacactgtcaaatcttctgccctggaccagaaacttcaggtctctcagacggagcctgcaaaatctgacttgtctaaactggaatcagttagaatgaaagtaccaaaggaaaaggggctttcaagccatgttgaagtggtggagaaggaaggcaggcttaaagccaggaagcacctcaagcctgagcagcctgcagatggggtaagtgctgtggatctggagaagctggaagccaggaaaaggcgctttgcagattccaatttaaaagcagaaaagcaaaaaccagaggtcaagaaaagcagtccagagatggaggatgctcgcgtgctttcaaaaaagcagcctgacgtgtcctctagagaggtcattctgctgagggaaggagaggctgaaagaaagcctgtgaggaaagaaattcttaaaagagaatctaaaaaaatcaaactggacagacttaatactgttgccagccccaaagactgtcaggagcttgccagtatttctgttgggtctggctcaaggcccagctcagacctacaagcaagactgggagaactagcaggtgaatctgtggaaaatcaagaagtccaatcaaaaaagcccattccctcaaaaccacagctcaaacagctgcaggtattagatgatcaaggaccagagagagaagacgttaggaaaaactattgcagtcttcgtgatgaaacacctgaacgtaaatcaggccaagagaaatcacattcagtaaatactgaagaaaaaattggcattgacatcgatcacacgcagagttaccgaaaacaaatggaacagagtcgtaggaaacagcagatggaaatggaaatagccaagtctgagaagtttggcagtcctaaaaaagatgtagatgaatatgaaagacgtagcctcgttcacgaggtaggcaaaccccctcaagatgtcactgatgactctcctcctagcaaaaagaaaaggatggatcatgtcgattttgatatctgcaccaagcgagaacggaattacagaagttcacgccaaatcagcgaagattctgaaaggactggtggttctcccagtgtccgacatggttccttccatgaagatgaggatcccataggctcccctaggctactgtcagtaaaagggtctcctaaagtagatgaaaaagtcctcccctattctaacataacagtcagggaagagtctttaaaatttaatccttatgattctagcaggagagaacagatggcagatatggccaaaataaaactatctgtcttgaattctgaagatgaactaaatcgttgggactctcagatgaaacaggatgctggcagatttgatgtgagtttcccaaacagcataattaagagagatagccttcgaaaaaggtctgtacgagatctggaacctggtgaggtgccttctgattctgacgaagatggtgaacacaaatcccactcacccagagcctctgcattatatgaaagttctcgattgtcttttttattgagggacagagaagacaagctacgtgagcgagatgaaagactctctagttctttagaaaggaacaaattttactcttttgcattggataagacaatcacaccagacactaaagctttgcttgaaagagctaaatccctctcttcatctcgtgaagaaaattggtcttttcttgattgggactcccgatttgcaaattttcgaaacaacaaagataaagaaaaggttgactctgctccaagacctattccatcctggtacatgaaaaagaagaaaattaggactgattcagaagggaaaatggatgataagaaagaggaccataaagaagaagagcaagagaggcaggaattgtttgcttctcgttttttacacagctcaatctttgaacaagattccaagcgattgcagcatctagagagaaaagaggaagattctgacttcatttctggtaggatctatgggaagcagacatctgagggagcaaacagcacaactgattccattcaagaaccagtagttctgttccatagcagatttatggagctcacacggatgcaacagaaagaaaaagaaaaagaccagaaacccaaagaggttgagaaacaggaagatacagagaatcatcccaagaccccagaatctgctcctgagaataaagattcagaactgaaaactccaccttccgttgggcctccaagtgtcacagtcgtaactctagaatcagccccatcagcactagagaagaccactggtgacaaaacggtagaggcgcctttggtaacagaagagaagactgtggagccagctaccgtctcagaagaagcaaagcctgcatctgaacctgctcctgcccctgtggaacagctggaacaagtagacctgcccccaggagcagaccccgataaagaagctgccatgatgcctgcgggtgttgaggaaggttcatcaggtgaccagccgccttatctggatgccaagcctccaactcccggggcctcgttttcccaggcagagagcaacgtagatccagagcctgacagtacccagccactttcaaaaccagctcagaagtctgaggaagccaatgagccaaaggccgaaaagccagacgccactgcagatgctgagcctgatgcaaaccagaaagccgaagctgctcctgagtctcagcccccagcttctgaagatttagaggttgatcctccagttgctgcaaaggataaaaagccaaacaaaagcaagcgttcaaagacccctgttcaggcagctgcagtgagtatcgtggagaagcccgtcacaaggaagagtgagaggatagaccgggaaaaactcaagcggtccaattctcctcggggagaagcacagaagcttttggaattgaagatggaggcagagaagattacaaggactgcttctaaaaactctgctgcagaccttgaacatcccgaaccaagtttgcctctcagccgaacaaggcgccggaatgtaaggagcgtctatgcaaccatgggtgaccatgaaaaccgctctcctgtcaaagagcccgttgagcaaccaagagtgaccagaaagagattggagcgagagcttcaggaggctgcagcggttcccaccacccctcggaggggaaggcctccaaagacacgccggcgagccgatgaagaggaggagaacgaggccaaggaacctgcagaaacactcaagccacctgagggatggcggtcgccaaggtcccagaaaactgcagctggtggtggaccccaagggaaaaagggaaaaaatgaaccgaaggtggatgctacacgtcctgaggccaccactgaggtgggcccccaaataggcgtgaaagagagctccatggaacccaaggctgctgaggaggaggcagggagtgaacagaaacgtgacagaaaagatgctggcacagacaaaaacccccctgaaaccgcccctgttgaagttgtagagaaaaaaccggcccctgaaaaaaactccaaatcaaagagaggaagatctcgaaactccaggttagcagtggacaaatctgcaagtctgaaaaatgtggatgctgctgtcagtcccaggggggctgcagcacaggcaggggagagggaatctggggtggtggcagtctcccctgagaaaagtgagagtccccaaaaggaggatggtttatcatcccagttgaaaagtgatccagttgatccagacaaggaaccagagaaagaagacgtgtctgcctctgggccgtccccagaagccacccagttagccaagcagatggagctggagcaggccgtggaacacatcgcaaagctcgctgaggcctctgcctctgctgcctataaggcagatgcaccagagggccttgccccagaggacagggacaagcctgcacaccaagcaagtgaaacagagctggctgcggccatcggctccatcatcaatgacatttctggggagccagaaaacttcccagcacctccaccttatcctggagaatcccagacagatctgcaaccccccgcaggtgcacaggcgctgcagccttctgaggaaggaatggagacagatgaggctgtatctggcatcctggaaactgaggctgctacagaatcttctaggcctccagtcaatgctcctgacccctcagccggcccaacagataccaaggaagccagaggaaatagcagtgaaacctcacactcagtgccagaagccaaagggtctaaagaagtggaagtcactcttgttcggaaagacaaagggcgccagaaaacaacccgatcacgccgcaagcgaaacacaaacaagaaagtggtggctcctgtagagagccatgtccctgaatccaaccaagctcaaggtgagagtcctgctgcaaatgaggggacaacagtacagcaccccgaagccccacaggaagaaaagcagagtgagaaaccccattccactcctcctcagtcatgtacttctgacctaagcaagattccctccacagagaattcgtcccaagaaatcagtgttgaggaaaggactccaaccaaagcatctgtgcccccagaccttcccccacctccccagccagcaccggtggatgaggagcctcaagccaggttcagggtgcattccatcattgaaagtgacccggtgaccccacccagcgatccaagcatccccatacccacactgccttctgtaactgcagcaaagctctcacctcctgtcgcctctggggggatcccacaccagagcccccctactaaggtgacagagtggatcacaaggcaggaggagccacgggctcagtctactccatctccagctcttcccccagacacaaaggcctctgatgttgacaccagctccagcaccctgaggaagattctcatggaccccaagtatgtgtctgccacaagtgtcacttccacaagtgtcaccacagccattgcagagcctgtcagtgctgccccttgcctacatgaggccccgcccccgccagttgactctaaaaagcctttagaagaaaaaacagcacctccagtgacaaacaactctgagatacaagcctcggaggtgctggtagctgctgacaaggaaaaggtggctccagtcattgctcccaaaattacctctgttattagccggatgcctgtcagcattgacctggaaaattcacagaagataaccttggcaaaaccagctcctcaaaccctcactggtctggtgagcgcactcactggcctggtgaacgtctccctggtcccggtgaatgccctgaaaggccccgtgaagggctcagtgaccacactgaaaagtttggtgagcacccctgctgggcccgtgaacgtcctgaaagggcctgtgaatgttcttacggggccagtgaatgttctcaccactccagtgaacgccacggtgggcacagtgaatgccgccccaggcacagtcaatgccgctgcgagtgcagtgaatgccacagcaagtgcagtgaccgtcacagcgggtgcggttactgctgcatctggtggtgtaacggccacaacaggcacggtgacaatggcaggggcagtgattgcgccgtcaacaaagtgcaaacagagagcgagtgctaatgaaaacagtcggttccacccagggtccatgcctgtgatcgacgatcgtccggcagacgcgggctcaggggcggggctgcgtgtgaacacttctgaaggggttgtgctcctgagttactcagggcagaagaccgaaggcccacagcggatcagcgccaagatcagccagatccccccggccagtgcaatggacattgaatttcagcagtcagtgtccaagtcccaggtcaaacctgattctgtcacagcatcgcagcctccatccaaaggccctcaagctcctgcaggctatgcgaacgtggccacccattccacgttggtactgaccgcccagacatataatgcctctcctgtgatttcgtctgtgaaggccgataggccatccttggagaagcccgagcccattcacctctcggtgtccacgcctgtcacccagggaggcacagtgaaggttctcacccaggggatcaacacaccccctgtgctggttcacaaccagctggtcctcaccccaagcattgtcaccacaaacaagaagcttgctgaccccgtcacccttaaaatcgagaccaaggtccttcagccggccaacctggggtccacgctcacgccccaccaccctcctgctctgcccagcaaactgcctacagaagtcaaccatgtcccctcggggcccagcatcccagcagatcgaactgtctcccatttggcagctgcaaagctagatgctcattctcctcgaccaagtggacccgggccatcctcattcccaagggcaagccaccccagcagtactgcatctacggcgctctccaccaacgccacagtcatgctggctgcaggcatcccagtgccccagttcatctccagcatccacccagagcagtctgtcatcatgccaccccacagcatcacccagactgtgtccctgagccacctctcccagggcgaggtgagaatgaacactcccacgctgcccagtatcacctacagcatccggccagaagcgcttcactctcctcgggctccgctgcagccccagcaaatagaggtcagggccccacagcgtgccagcaccccgcagccagccccagctggtgtgcctgcactggcctcccagcaccctcccgaggaggaagtgcattatcaccttcctgtcgctcgagccacagcccctgtgcagtcagaggtactagtcatgcagtctgagtaccgactgcacccctatactgtgccacgggatgtgaggatcatggtgcatccacatgtgacggcagtcagcgagcagcccagggccgcggatggggtggtgaaggtgccaccagccagcaaggcccctcagcagccagggaaggaagctgccaagacaccagatgccaaagctgcccccacccccacccctgcccccgtccctgtccctgtcccccttcctgcccctgctcctgcccctcatggtgaggcccgtatcctcacagttacccccagtaaccaactccaggggctgcctctgacccctcctgtggtggtgacccatggggtgcagattgtgcactccagcggggagctgtttcaagagtaccggtacggcgacatccgcacctaccaccccccggcccagctcacacacactcagtttcccgccgcttcctctgttggcctgccttcccggaccaagacagctgctcagggccctcctcctgaaggtgagcccctgcagcctcctcagcctgtgcagtccacacagcctgcccagcctgcaccaccctgcccgccctcccagctcggtcagcccggccagccaccaagcagcaagatgcctcaagtgtcccaggaggcaaaggggacccagacgggagtagagcagcctcgcctcccagctggacctgcaaacaggccacctgagcctcacacccaggttcagagggcacaagcagaaacaggcccgacttccttcccctcccctgtgtctgtctccatgaagcctgaccttccagtctctcttcccactcagactgccccaaaacagccgttgtttgtcccaacaacctctggccccagcaccccaccaggactggttctgccacacactgaattccagccagcccccaaacaagattcctctccacacctgacttcccagagacccgtggatatggttcaacttctgaagaagtaccccatcgtgtggcagggcctgctggccctcaagaatgacacagctgctgtgcagctccacttcgtctctggcaacaacgtcctggcccatcggtccctgcccctttctgaaggagggcccccactaaggatcgcccagaggatgcggctggaggcaacgcagctggaaggggttgcccgaaggatgacggtggagacagattactgtctgctgctggctctgccctgtggccgtgaccaagaggatgttgtgagccagaccgagtccctcaaggctgccttcatcacttacctgcaggccaagcaggcggcagggatcatcaacgttcccaaccctggctccaatcagcctgcctacgtgctgcagatcttcccgccctgtgagttctctgagagtcacctgtcccgcctggcccctgacctccttgccagcatctccaacatctctccccacctcatgattgtcattgcctccgtgtgagccactgagtggttatcacctcagtgaatcttcccagggctctgcagtaaaaacaaaggacaacccagccaagcagaggaagaagctgccgaaggggacagactccactgccagacggccagccgtttgctgtcctgccgcccggctcagtcggccagacttcctctaggagtggtgctgctaccttgtatgtttacataatgctttagcccaaggacacatcaccaacccatggactcgcagacaccggggctgggtttctctttcctctttttggagaaaaggaacagggcagtggaatgaaaattttttgtttgtttgtttttaagaaacaagaaaacagaactgcctttgcactaaattagtgacttggacttttgcccagtgaagacaggctgtgacactctggatgtcttggtgtgtgtagacacacattgcagactcttaacgcaggaaggacttcaaacttctgctgagaccttggggtcaaggaacatttcattggttttttttgtccacccccatctcccttgctcatttggatgcgtcaccttaattctcctgctgccaccgtctttgattcaccgggatgtacagtttacagttgaagagcaaacagaaaggttttctcttggtgggatatgcagaacttgggatgtgtgtatatataaatatataatatatataaatatatataatactgacttaaaaaatcaaatcccccgacatacgttttttttaatctgtgccaaaaatgtgttttcagaggaaatcttattttcatattcagactttgtattgcccactcatttgtataagtgcgcttcggtacagcacgggtcctgctcccgcgatgtggaagtgtcacacggcacctgtacaaaaagactggctaacccctcttcctattaccttgatctcttcccccaacttcctaacacttattaatttatgaaactgtttttctcagcgcagttttgttttgtgtgtccattggattacaaactttattaaaaaatataaaacac

SEQ ID NO: 10 - DNA, 2102 bp, Homo sapiens
cctgcgacttctctaatgtctgctttggctgcccggctgctgcagcccgcgcacagctgctcccttcgccttcgccctttccacctcgcggcagttcgaaatgaagctgttgtcatttctggaaggaaactggcccagcagatcaagcaggaagtgcggcaggaggtagaagagtgggtggcctcaggcaacaaacggccacacctgagtgtgatcctggttggcgagaatcctgcaagtcactcctatgtcctcaacaaaaccagggcagctgcagttgtgggaatcaacagtgagacaattatgaaaccagcttcaatttcagaggaagaattgttgaatttaatcaataaactgaataatgatgataatgtagatggcctccttgttcagttgcctcttccagagcatattgatgagagaaggatctgcaatgctgtttctccagacaaggatgttgatggctttcatgtaattaatgtaggacgaatgtgtttggatcagtattccatgttaccggctactccatggggtgtgtgggaaataatcaagcgaactggcattccaaccctagggaagaatgtggttgtggctggaaggtcaaaaaacgttggaatgcccattgcaatgttactgcacacagatggggcgcatgaacgtcccggaggtgatgccactgttacaatatctcatcgatatactcccaaagagcagttgaagaaacatacaattcttgcagatattgtaatatctgctgcaggtattccaaatctgatcacagcagatatgatcaaggaaggagcagcagtcattgatgtgggaataaatagagttcacgatcctgtaactgccaaacccaagttggttggagatgtggattttgaaggagtcagacaaaaagctgggtatatcactccagttcctggaggtgttggccccatgacagtggcaatgctaatgaagaataccattattgctgcaaaaaaggtgctgaggcttgaagagcgagaagtgctgaagtctaaagagcttggggtagccactaattaactactgtgtcttctgtgtcacaaacagcactccaggccagctcaagaagcaaagcaggccaatagaaatgcaatatttttaatttattctactgaaatggtttaaaatgatgccttgtatttattgaaagcttaaatgggtgggtgtttctgcacatacctctgcagtacctcaccagggagcattccagtatcatgcagggtcctgtgatctagccaggagcagccattaacctagtgattaatatgggagacattaccatatggaggatggatgcttcactttgtcaagcacctcagttacacattcgccttttctaggattgcatttcccaagtgctattgcaataacagttgatactcattttaggtaccagaccttttgagttcaactgatcaaaccaaaggaaaagtgttgctagagaaaattggggaaaaggtgaaaaagaaaaaatggtagtaattgagcagaaaaaaattaatttatatatgtattgattggcaaccagatttatctaagtagaactgaattggctaggaaaaaagaaaaactgcatgttaatcattttcctaagctgtccttttgaggcttagtcagtttattgggaaaatgtttaggattattccttgctattagtactcattttatgtatgttacccttcagtaagttctccccattttagttttctaggactgaaaggattcttttctacattatacatgtgtgttgtcatatttggcttttgctatatactttaacttcattgttaaatttttgtattgtatagtttctttggtgtatcttaaaacctatttttgaaaaacaaagttggcttgataatcatttgggcagcttgggtaagtacgcaacttacttttccaccaaagaactgtcaccacctgcctgcttttctgtgatgtatgtatcctgttgacttttccagaaattttttaagagtttgagttactattgaatttaatcagactttctgattaaagggttttctttcttttttaataaaacactctgtctggtgtggtatgaatttctg

SEQ ID NO: 11 - DNA, 3081 bp, Homo sapiens
cccgccagcccagcccagcccaaccctactccctccccacgccagggcagcagccgttgctcagagagaaggtggaggaagaaatccagaccctagcacgcgcgcaccatcatggaccattatgattctcagcaaaccaacgattacatgcagccagaagaggactgggaccgggacctgctcctggacccggcctgggagaagcagcagagaaagacattcacggcatggtgtaactcccacctccggaaggcggggacacagatcgagaacatcgaagaggacttccgggatggcctgaagctcatgctgctgctggaggtcatctcaggtgaacgcttggccaagccagagcgaggcaagatgagagtgcacaagatctccaacgtcaacaaggccctggatttcatagccagcaaaggcgtcaaactggtgtccatcggagccgaagaaatcgtggatgggaatgtgaagatgaccctgggcatgatctggaccatcatcctgcgctttgccatccaggacatctccgtggaagagacttcagccaaggaagggctgctcctgtggtgtcagagaaagacagccccttacaaaaatgtcaacatccagaacttccacataagctggaaggatggcctcggcttctgtgctttgatccaccgacaccggcccgagctgattgactacgggaagctgcggaaggatgatccactcacaaatctgaatacggcttttgacgtggcagagaagtacctggacatccccaagatgctggatgccgaagacatcgttggaactgcccgaccggatgagaaagccatcatgacttacgtgtctagcttctaccacgccttctctggagcccagaaggcggagacagcagccaatcgcatctgcaaggtgttggccgtcaaccaggagaacgagcagcttatggaagactacgagaagctggccagtgatctgttggagtggatccgccgcacaatcccgtggctggagaaccgggtgcccgagaacaccatgcatgccatgcaacagaagctggaggacttccgggactaccggcgcctgcacaagccgcccaaggtgcaggagaagtgccagctggagatcaacttcaacacgctgcagaccaagctgcggctcagcaaccggcctgccttcatgccctctgagggcaggatggtctcggacatcaacaatgcctggggctgcctggagcaggtggagaagggctatgaggagtggttgctgaatgagatccggaggctggagcgactggaccacctggcagagaagttccggcagaaggcctccatccacgaggcctggactgacggcaaagaggccatgctgcgacagaaggactatgagaccgccaccctctcggagatcaaggccttgctcaagaagcatgaggccttcgagagtgacctggctgcccaccaggaccgtgtggagcagattgccgccatcgcacaggagctcaatgagctggactattatgactcacccagtgtcaacgcccgttgccaaaagatctgtgaccagtgggacaatctgggggccctaactcagaagcgaagggaagctctggagcggaccgagaaactgctggagaccattgaccagctgtacttggagtatgccaagcgggctgcacccttcaacaactggatggagggggccatggaggacctgcaggacaccttcattgtgcacaccattgaggagatccagggactgaccacagcccatgagcagttcaaggccaccctccctgatgccgacaaggagcgcctggccatcctgggcatccacaatgaggtgtccaagattgtccagacctaccacgtcaatatggcgggcaccaacccctacacaaccatcacgcctcaggagatcaatggcaaatgggaccacgtgcggcagctggtgcctcggagggaccaagctctgacggaggagcatgcccgacagcagcacaatgagaggctacgcaagcagtttggagcccaggccaatgtcatcgggccctggatccagaccaagatggaggagatcgggaggatctccattgagatgcatgggaccctggaggaccagctcagccacctgcggcagtatgagaagagcatcgtcaactacaagccaaagattgatcagctggagggcgaccaccagctcatccaggaggcgctcatcttcgacaacaagcacaccaactacaccatggagcacatccgtgtgggctgggagcagctgctcaccaccatcgccaggaccatcaatgaggtagagaaccagatcctgacccgggatgccaagggcatcagccaggagcagatgaatgagttccgggcctccttcaaccactttgaccgggatcactccggcacactgggtcccgaggagttcaaagcctgcctcatcagcttgggttatgatattggcaacgacccccagggagaagcagaatttgcccgcatcatgagcattgtggaccccaaccgcctgggggtagtgacattccaggccttcattgacttcatgtcccgcgagacagccgacacagatacagcagaccaagtcatggcttccttcaagatcctggctggggacaagaactacattaccatggacgagctgcgccgcgagctgccacccgaccaggctgagtactgcatcgcgcggatggccccctacaccggccccgactccgtgccaggtgctctggactacatgtccttctccacggcgctgtacggcgagagtgacctctaatccaccccgcccggccgccctcgtcttgtgcgccgtgcccacagatgtgaaatgaatgtaatctaatagaagcctaatcagcccaccatgttctccactgaaaaatcctctttctttggggtttttctttctttcttttttgattttgcactggacggtgacgtcagcctgtacaggctcccaggggtggcgtcaaatgctattgaaattgcgctgaatcgtatgctttttccttttgataaataaacaatgtaaaaatgtttcaaaaacctaataaaataaataaatacg

SEQ ID NO: 12 - DNA, 990 bp, Homo sapiens
cccatcccgtgggtggccggcgacaagatggcagcagcgtgtcggagcgtgaagggcctggtggcggtaataaccggaggagcctcgggcctgggcctggccacggcggagcgacttgtggggcagggagcctctgctgtgcttctggacctgcccaactcgggtggggaggcccaagccaagaagttaggaaacaactgcgttttcgccccagccgacgtgacctctgagaaggatgtgcaaacagctctggctctagcaaaaggaaagtttggccgtgtggatgtagctgtcaactgtgcaggcatcgcggtggctagcaagacgtacaacttaaagaagggccagacccataccttggaagacttccagcgagttcttgatgtgaatctcatgggcaccttcaatgtgatccgcctggtggctggtgagatgggccagaatgaaccagaccagggaggccaacgtggggtcatcatcaacactgccagtgtggctgccttcgagggtcaggttggacaagctgcatactctgcttccaaggggggaatagtgggcatgacactgcccattgctcgggatctggctcccataggtatccgggtgatgaccattgccccaggtctgtttggcaccccactgctgaccagcctcccagagaaagtgtgcaacttcttggccagccaagtgcccttccctagccgactgggtgaccctgctgagtatgctcacctcgtacaggccatcatcgagaacccattcctcaatggagaggtcatccggctggatggggccattcgtatgcagccttgaagggagaaggcagagaaaacacacgctcctctgcccttcctttccctggggtactactctccagcttgggaggaagcccagtagccattttgtaactgcctaccagtcgccctctgtgcctaataaagtctctttttctcacaaaaaaaaaaaaaaaaaaaaaaaaaaaaaaaaaaa

SEQ ID NO: 13 - DNA, 1052 bp, Homo sapiens
aaagaattcggcacgagcacaatggaggactccggaaagactttcagctccgaggaggaagaagctaactattggaaagatctggcgatgacctacaaacagagggcagaaaatacgcaagaggaactccgagaattccaggagggaagccgagaatatgaagctgaattggagacgcagctgcaacaaattgaaaccaggaacagagacctcctgtccgaaaataaccgccttcgcatggagctggaaaccatcaaggagaagtttgaagtgcagcactctgaaggctaccggcagatctcagccttggaggatgacctcgcgcagaccaaagccattaaagaccaattgcagaaatacatcagagagctggagcaagcaaatgacgccctggaaagagccaagcgcgccacgatcatgtctctcgaagactttgagcagcgcttgaatcaggccatcgaaagaaatgccttcctggaaagtgaacttgatgaaaaagagaatctcctggaatctgttcagagactgaaggatgaagccagagatttgcggcaggaactggccgtgcagcagaagcagaagaaacccaggacccccattcccagctcagtggaagctgagagtacagacacagctgtgcaggccacgggctccgtgccgtccacgcccattgctcaccgaggacccagctcaagtttaaacacacctgggagcttcagacgtggcctggacgactccaccgggggacccccctcacactgcggcccggatatcagccctcaacattgtgggagacctactgcggaaagtcggggcactggagtccaaactcgcttcctgccggaacctcgtgtacgwtcagtccccaaaccgaacaggtggcccagcctctgggcggagcagcaagaacagagatggcggggagagacggccaagcagcaccagcgtgcctttgggtgataagggktcdgtaccttctaataaacctctcgctggcggggagaacccgcctgccccaggcaagakahactcacaaaaaaaaaaaaaaaaac

SEQ ID NO: 14 - DNA, 1209 bp, Homo sapiens
gaattcctgacttccttttcggaggaagatccttgagcagccgacgttgggacaaaggatttggagaaacccagggctaaagtcacgtttttcctcctttaagacttacctcaacacttcactccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaaggcaccttcgtggagcgggaccgcaagcgggagaagaggaagcccaagagccaggagaccccggccaccaagaaggctgtgcaaggcgggggagccacccccgtggtgggggctgtccaggggcctgtcccgggcatgccgccgatgactcaggcgccccgcattatgcaccacatgccgggccagccgccctacatgccgccccctggtatgatccccccgccaggccttgcacctggccagatcccaccaggggccatgcccccgcagcagcttatgccaggacagatgccccctgcccagcctctttctgagaatccaccgaatcacatcttgttcctcaccaacctgccagaggagaccaacgagctcatgctgtccatgcttttcaatcagttccctggcttcaaggaggtccgtctggtacccgggcggcatgacatcgccttcgtggagtttgacaatgaggtacaggcaggggcagctcgcgatgccctgcagggctttaagatcacgcagaacaacgccatgaagatctcctttgccaagaagtagcaccttttccccccatgcctgccccttcccctgttctggggccacccctttcccccttggctcagccccctgaaggtaagtccccccttgggggccttcttggagccgtgtgtgagtgagtggtcgccacacagcattgtacccagagtctgtccccagacattgcacctggcgctgttaggccggaattaaagtggctttttgaggtttggtttttcacaaaaaaaaggaattc

SEQ ID NO: 15 - DNA, 1414 bp, Homo sapiens
gggtttgaagaccttcagcgttgccctggcggacagagacaggccctcggggtggaggtctttggtttcataagagcctgagagagatttttctaagatatgtgtaacacaccaacgtactgtgacctaggaaaggctgctaaggatgtcttcaacaaaggatatggctttggcatggtcaagatagacctgaaaaccaagtcttgtagtggagtggaattttctacttctggtcatgcttacactgatacagggaaagcatcaggcaacctagaaaccaaatataaggtctgtaactatggacttaccttcacccagaaatggaacacagacaatactctagggacagaaatctcttgggagaataagttggctgaagggttgaaactgactcttgataccatatttgtaccgaacacaggaaagaagagtgggaaattgaaggcctcctataaacgggattgttttagtgttggcagtaatgttgatatagatttttctggaccaaccatctatggctgggctgtgttggccttcgaagggtggcttgctggctatcagatgagttttgacacagccaaatccaaactgtcacagaataatttcgccctgggttacaaggctgcggacttccagctgcacacacatgtgaacgatggcactgaatttggaggttctatctaccagaaggtgaatgagaagattgaaacatccataaaccttgcttggacagctgggagtaacaacacccgttttggcattgctgctaagtacatgctggattgtagaacttctctctctgctaaagtaaataatgccagcctgattggactgggttatactcagacccttcgaccaggagtcaaattgactttatcagctttaatcgatgggaagaacttcagtgcaggaggtcacaaggttggcttgggatttgaactggaagcttaatgtggtttgaggaaagcatcagatttgtccctggaagtgaagagaaatgaacccactatgttttggccttaaaattcttctgtgaaatttcaaaagtgtgaactttttattcttccaaagaattgtaatcctccccacactgaagtctaggggttgcgaatccctcctgagggagatgcttgaaggcatgcctggaagttgtcatgtttgtgccacgtttcagttcagttctgaagtgttattaaatgtgttcctcagcgacagtgtagcgtcatgttagaggagacgatctgacccaccagtttgtacatcacgtcctgcatgtcccacaccattttttcatgaccttgtaatatactggtctctgtgctatagtggaatctttggttttgcatcatagtaaaataaaataaacccatcacatttggaacataaaaaaaaaaaaaaaaaaaaaaaaaaaaaa

SEQ ID NO: 16 - DNA, 7799 bp, Homo sapiens
ggatcctgtctctacaaataatacaaaaattagctgggtttagtggcatgcctgtactcccagctacttggaaggctaaggcaggaggatcacttgaactcaggagttcaaggctgcagtgagacacgattgtgcaactgccctccagcctaggcaacagagcaagattctgcctcaaaaaaaacaaaacaggccaggcgtggtttctcatgcctgtaatcccaacactttgataggctgaggcaagtggatcacctgaggtcagcagttggagaccagcctggccaacatagtgaaaccctgtctctactaaaaatacaaaaattagctgggcatggtggcacacacctgtagtcccagctacttgggaggctgaggtaggagaatcgcttgaacccaggaggtggaggttgcagtgagctgagatggcgccaccgcactccagcctgggcgacagagcgagactccatctcaaaaaaaaaaaaaaaaagggaaaattaaaacgaaaaaatggccctgccatcagtctcagaaccctgggttaaggttgccctccaccccgcatctataagaattagctattgactctgctttagcagtccagaaaggagggaaaagatcatctgcccctgccagttagtcatagcattttcaagcactccctagttacctagttacatttgtttgttgtcactttggaaagtttatgttccagatcacatttttggcttctgctgcaaatccttttgagtcataacagggattattaaataaagttaaggtttgttttcttcatctcaaactaacctttcagactttgaaaatcaaactgatttcttttcagataaatagagcttttttaaaaatacagcttttaagctggatgcagtggcatgtacctgtagtcccagctaatcagaagactaaggcaagaggctcacttgagcccagaagttcaagtccagcttaggcaacatagtgagaccctgtctctaaaaattaaagtaataataatccttattttaaaaactgagctgagtgtggtggttcatgcctgtaatcccagcactttgggaggccaaggcaagtggatcacttgaagtcaggagttcaagaccagccatctggccaacatggcgaaaccccaactaaaaatacaaaaattagctggtcgtggtggtacacacctgtaaccccagctactcgggaggctgaggctggagaatcacttgaaccctggaggtggaggttgcagtgagccccgagattggcgccactgtactccagcctgggcaacagagtgagacttcgtctcaaaataaataaataaatacaaactgttattgccagatgcagtggttcacacctgtaatttcggcactttgggaggcctaggcgggtggatcgcttccactcaagagttcgagaccagcctgggcaacatggtgaaaccccatctcttaaaaaaaaattcaaaaattagctgggcgtggtggcatgcacctgcaatcccagctacttgggagactgagatgggaggatcacctgagccccaggaaagtcataatcacactgttgcactccagcctgggcaacagtgaaaccctatatccgaaaacaaaacaaaaccaaaccctgccataagcttagaaatgtttaaagagaaagttaaaactcaccccaaatcccacatggagatgagaatctggctacttctgtctctcctagtttcggagcaacatcccctgtcacctggttgatatctcttgcggattaccctgcagtgccacgctaccatgtgggatgcacaaatgtcagagactcagtcacaaaggggagtgtcttgtggatgagccctgcaagcagccctgcaccacccccagagctgactgtggtcacccgtgtatggcaccctgccataccagctcaccctgccctgtgactgcttgtaaagctaaggtgggtatttctggccacagatgcagcattgactgtagttctgaggctactagtgaatccagaactgtctacagtggcccctgggcatgcatcttaattaagggtcattggttgcaaataaccacaagttagagaaaggtagagtaagtcatacaagtttctgcctgcccaacttggtatggccttttgattcagcaggtacccactgccaccggaccacagtctgtgtctcccttgggtgagattctcaatagtcactgattgtttactccatttatttaataaatgtattaaatgtcttttgtgcaagacatagtatgaggtgctagggataaattagcgaacaagagacacatggttcctcacaatctgataggacagatacttgattattacttagataactaattatggctgtggtgtcatgagggagtagtttaagccttgtggagggggctatgagagcaggtaaatgggaggttaagcctggtctaaaggatcaagtgtggccaccttgaggatgtggagttggcttggagttggctaggccacagttgggcctagggcaagtgtctgtctctgatggtacaaacttcatcctaggcctgccccttcagccaggattaagaacagaggtggtttccaaaagggggcattgtggacggggcaattctttcctagcatgtcaacaactgtgtcagtttgcatgtgtggttagcttcttccagcttggagagtggccacccactgactctctagtctctgctatggtttaagagaggatttaagagtgaaaagtgctttattcacatagttccagtgtgctaaaagtcgttccatgttcatctgacttcaggtagagctacagtgtgaatgtggacgaagaaaagagatggtgatttgctctgaagcatctagtacttatcaaaggttagtgttacttaaatgttaacaactgatggcctaggtgaaacagatacatgtgttttgttttgtttttaaagctagtcaagtgaagcagtgggagtggagaagaaacgaagtctgtaactgttgtaatcagttgtaaacaccactgcacagatacatgtgtttatcatagtatccttgcaacttttctaaaagctggaaaggtttttttgtttgttttgttttgttttattgagaggggtctcgccctgtttcccaggctggtctcccaccttggcctcccaaagtgctgggatcacaggcgtgagccactacacctggctggaaagtttcttacatacatttttaaaaatctggtttctctagttcaaagaaacagtgctgctttatataccttttcttccctaactttagtgtgtaaaataactttgaaatttctccgcttcctccttcctctttctttttttctctcttttggtcgtattacattgtgagataagaagggaagatgtcctgaagttaggtgaattgctttcacatcctaccatttgtcattgacacagccagggctaagctctgtgctcctctggtgtgagggtttctaattgttctgtgaggtatctctgcagcctcctgaagtcttgggacctgtgtagccgtacagggatctgagtgctgagatggaggtgttagagaatgaatggctatatatatatatatctggctagatggggagaggtgccatggatataggactttaagaggatgcacacatcagattggctgtttttgaatacctactaagtgcaagtgttgctctaggggatactgagggattgagtcacctcatcgcttcccatgtagctgctttgaatataattgaagagatatgacataaatatgtaggtagttcatagtagccgggcttccaaggccacaccagttttcatctgatagatttgctttttttttttttttttttttggaaatggagtttcgctcctgttgcccaggttagagtgaaatggtgaaatctcggttcactgaaacctgaacctcccaggttcaagtgattctcctgcttcagcctcctgagtagcgggggatcacaggcacctgctaccatgcctggctaattttttgtttttttagtagagacggggtttcaccatattggccaggctggtcatgaactcctgacctcaggtgatccacctgccttggtttcccaaagtgctgggcttacaggcgtgagccaccacacctggcctagatttgcttttaaagaggtcttctgtatcccaagtataaggaggttcttgtgaaactgcaatgcctctttttccctttcttttttatatttcagaatagctgcaatctccatggcctctaagataacagacatgcagcttggaggttcagtggagatcagcaagttaattaccaaaaaggaagttcatcaagccaggtaatttttaaaatgcatatatgtgccttctttcttcaattagacttcaattagagggcagagattattcatttatctcaatccttcatagtagagccttgcatacacaataagtattcaatagacaatttgatgttgagacatctgctccatccccaatgaatcctactcaggagatggctaagactaaaggagacttttccaggcctcacagtagccgaaggaagcagagacctataggtgtttagcttaacccatatgagaatagagaacccagagaggtggccatctctggagagggagattgggctgttaagaggggcacctgtggaacatttggggctgagcagccagggaacactctgaggccactggaggactcctagaccccataacagagaggccatcatcccacctgtccttcagagggacctcatccagccattcctggattggggtccctggggaaatggtatttctagggcaaggttactaatggaaactgaagagcaggtggcctcacaggctgcttcctggcctgagggattttttttggcaagagtcagcggcttccaggttgactgagacttccttttcttgctggatgggagctgtacagtctgtattctgactttaattttttttcatttgcttatcaaggctggagtgtgatgaggagtgttcagccttggaaaggaaaaagtaagtagttgcagctgcttttttaatctccttgcccttgagctctgtgaaattaatgggagggagcaacccttcaaacgtcattcactcagccctgcgctgcgtcttttaccgttatcaaagagcaagagaaatgccgttaccaagaattgaattgtcttaaggataaaccactcaggtgatttcagcatattgagaaacatgagctgtgtggttttgggcatgatataaacatcacataacacagcatacttattcagggactggccaacttcctgactcagagataagaggtttaaaacaatgtttagttaacttgtcggtggagtttagcaaacatattgaagattggaagtgtgcagatcagtgaatcgtcactaaatgattacaggtgtgtaatcatttgtgtcactaaatgaaaacagttgagtatggatacaggtgaaaagataattaccagggccgtctcttggactctacataaaggtgaaacctctatcctaatttctctttagattagtttttggactttatgtaagtgatgaaatcatgtagtttgtattatttttgtctggcttctttcgttaaatttttgtgacaatcacccctgttgttgcatgtagccatgctttaatcaattttaatcctgcatgattttttttggattactaatcatttacccattaagctggtaatggatatcctttgattactggttggctattaccaaaaaaacttgttaggaatcttttttttttttttttttttgagatggagtttttctcttgtcgcctagcctggagtccaatgtcatgatcttggctccctcccacctctccctcccgggttcaatcgattctcttcccttaccctccagagtagttggcattacaggtgtgtcccaccatgcccagctgatttttgtatttttagtagaaatggggtttcaccatgttggccaggctggtctcgaactcctgcctcaggtgatccacctgcctcgacctcccaaagtgttgggattacaagcttgagccaccacgcccagcccagaacattcttaaacttgcttgctctatatttctgtacatttctgttggatataagccagaagtagaattgctgattaatagggtttatgtgtgttcagcattagtagattctgcccatgtccaaagtgattgtaccaaattacactcccaccagctaaggataattcccattgctgtacatttttgtcagcacttgaaattgtcactttttaattttagccattttgtagatgtgtataacaagatgtcattgagcttttaatttctattttcctgatgacttaagagattgagcacctttcttgtagttactggccattggatagcctcatttttgaagtgcctgtacaagtcctttgcttatttttttgtgctctttttgtattgattagtaggaattctttatgtattctgaatatgagtcctttgttggctctatgtatacattgtgagtactttgttgcactctgtaccttcattttttactctcttgatgcctttttttaatcccagaagctaaagaactcttgttgtcttttgataaacagaaatttttaatgttaatattatccagtttatttgagtttcttaataagcacttttcatgtcctatttagaaaaatttgtctatcctgaggcaatgacaatattctgttattatctagaaactgtgttattttacctttcacattttaaatctacagttgacctgtactgatttttttgagtatggataaggagagtttacaattcagcttttttccacgtgaatatccatttgacgagtaccaagattgtccttttcccagcactctgcagtgccacctctgtcatacatcacatataagcctgggcaacttggcaaaacctcatctgtacaaaaaataaaataaaattagccaggtgtggtggtgcacacctgtagtcccagctacttgggaggcttgaggtgggaagattgcttgaacctgcgaggttgaggctacagtgagccatgatcaccagtctggactatacttttggtgacagagtgaaatctgtctcaaaaaaaaaaaaaaaaaataagccgggcatggtggctcaccatgcccgtaatggtggtgcctataatcccagcactttgggaggttgaggtgggcagatcacaaggtcagcagatcgagaccatcctggtaaaaccccatctctactaaaaatacaaaaaattagccgggcatggtggcgggcacttgtagtcccagctactcgggaagctgaggcaggagaatggcatgaacccaggaggcggagcttgcagtgagccgagatggtgccactgcactctagcctgggcgacagtgggagactccgcatcaaaaaataataataaataaataaagtatatcaaatatatatatatatgtgagtctgtttctgagctcatcctgttcaactgcactagtatcacactgtcttaattactgtacctttataacaagtcttaatatctggtagtatatatcctcaaccatgacttttcttcaatattgtctttgattattctttattcttagccctttacatttccacttaatgtctttttcatttagtatttatttattgagataggatcttgctcgtcacccatgctggaagtagtggcacaatcatagttcacgtaccctggaactcctgggctcaagggatcc

SEQ ID NO: 17 - DNA, 5130 bp, Homo sapiens
atgggcttcttgcccaagcttctcctcctggcctcattcttcccagcaggccaggcctcatggggcgtctccagtccccaggacgtgcagggtgtgaaggggtcttgcctgcttatcccctgcatcttcagcttccctgccgacgtggaggtgcccgacggcatcacggccatctggtactacgactactcgggccagcggcaggtggtgagccactcggcggaccccaagctggtggaggcccgcttccgcggccgcaccgagttcatggggaaccccgagcacagggtgtgcaacctgctgctgaaggacctgcagcccgaggactctggttcctacaacttccgcttcgaaatcagtgaggtcaaccgctggtcagatgtgaaaggcaccttggtcacagtaacagaggagcccagggtgcccaccattgcctccccggtggagcttctcgagggcacagaggtggacttcaactgctccactccctacgtatgcctgcaggagcaggtcagactgcagtggcaaggccaggaccctgctcgctctgtcaccttcaacagccagaagtttgagcccaccggcgtcggccacctggagaccctccacatggccatgtcctggcaggaccacggccggatcctgcgctgccagctctccgtggccaatcacagggctcagagcgagattcacctccaagtgaagtatgcccccaagggtgtgaagatcctcctcagcccctcggggaggaacatccttccaggtgagctggtcacactcacctgccaggtgaacagcagctaccctgcagtcagttccattaagtggctcaaggatggggtacgcctccaaaccaagactggtgtgctgcacctgccccaggcagcctggagcgatgctggcgtctacacctgccaagctgagaacggcgtgggctctttggtctcaccccccatcagcctccacatcttcatggctgaggtccaggtgagcccagcaggtcccatcctggagaaccagacagtgacactagtctgcaacacacccaatgaggcacccagtgatctccgctacagctggtacaagaaccatgtcctgctggaggatgcccactcccataccctccggctgcacttggccactagggctgatactggcttctacttctgtgaggtgcagaacgtccatggcagcgagcgctcgggccctgtcagcgtggtagtcaaccacccgcctctcactccagtcctgacagccttcctggagacccaggcgggacttgtgggcatccttcactgctctgtggtcagtgagcccctggccacactggtgctgtcacatgggggtcatatcctggcctccacctccggggacagtgatcacagcccacgcttcagtggtacctctggtcccaactccctgcgcctggagatccgagacctggaggaaactgacagtggggagtacaagtgctcagccaccaactcccttggaaatgcaacctccaccctggacttccatgccaatgccgcccgtctcctcatcagcccggcagccgaggtggtggaaggacaggcagtgacactgagctgcagaagcggcctaagccccacacctgatgcccgcttctcctggtacctgaatggagccctgcttcacgagggtcccggcagcagcctcctgctccccgcggcctccagcactgacgccggctcataccactgccgggcccgggacggccacagtgccagtggcccctcttcgccagctgttctcactgtgctctacccccctcgacaaccaacattcaccaccaggctggaccttgatgccgctggggccggggctggacggcgaggcctccttttgtgccgtgtggacagcgacccccccgccaggctgcagctgctccacaaggaccgtgttgtggccacttccctgccatcagggggtggctgcagcacctgtgggggctgttccccacgcatgaaggtcaccaaagcccccaacttgctgcgtgtggagattcacaaccctttgctggaagaggagggcttgtacctctgtgaggccagcaatgccctgggcaacgcctccacctcagccaccttcaatggccaggccactgtcctggccattgcaccatcacacacacttcaggagggcacagaagccaacttgacttgcaacgtgagccgggaagctgctggcagccctgctaacttctcctggttccgaaatggggtgctgtgggcccagggtcccctggagaccgtgacactgctgcccgtggccagaactgatgctgccctttacgcctgccgcatcctgactgaggctggtgcccagctctccactcccgtgctcctgagtgtactctatcccccggaccgtccaaagctgtcagccctcctagacatgggccagggccacatggctctgttcatctgcactgtggacagccgccccctggccttgctggccttgttccatggggagcacctcctggccaccagcctgggtccccaggtcccatcccatggtcggttccaggctaaagctgaggccaactccctgaagttagaggtccgagaactgggccttggggactctggcagctaccgctgtgaggccacaaatgttcttggatcatccaacacctcactcttcttccaggtccgaggagcctgggtccaggtgtcaccatcacctgagctccaagagggccaggctgtggtcctgagctgccaggtacacacaggagtcccagaggggacctcatatcgttggtatcgggatggccagcccctccaggagtcgacctcggccacgctccgctttgcagccataactttgacacaagctggggcctatcattgccaagcccaggccccaggctcagccaccacgagcctagctgcacccatcagcctccacgtgtcctatgccccacgccacgtcacactcactaccctgatggacacaggccctggacgactgggcctcctcctgtgccgtgtggacagtgaccctccggcccagctgcggctgctccacggggatcgccttgtggcctccaccctacaaggtgtggggggacccgaaggcagctctcccaggctgcatgtggctgtggcccccaacacactgcgtctggagatccacggggctatgctggaggatgagggtgtctatatctgtgaggcctccaacaccctgggccaggcctcggcctcagctgacttcgacgctcaagctgtgaatgtgcaggtgtggcccggggctaccgtgcgggaggggcagctggtgaacctgacctgccttgtgtggaccactcacccggcccagctcacctacacatggtaccaggatgggcagcagcgcctggatgcccactccatccccctgcccaacgtcacagtcagggatgccacctcctaccgctgcggtgtgggcccccctggtcgggcaccccgcctctccagacctatcaccttggacgtcctctacgcgccccgcaacctgcgcctgacctacctcctggagagccatggcgggcagctggccctggtactgtgcactgtggacagccgcccgcccgcccagctggccctcagccacgccggtcgcctcttggcctcctcgacagcagcctctgtccccaacaccctgcgcctggagctgcgagggccacagcccagggatgagggtttctacagctgctctgcccgcagccctctgggccaggccaacacgtccctggagctgcggctggagggtgtgcgggtgatcctggctccggaggctgccgtgcctgaaggtgcccccatcacagtgacctgtgcggaccctgctgcccacgcacccacactctatacttggtaccacaacggtcgttggctgcaggagggtccagctgcctcactctcattcctggtggccacgcgggctcatgcaggcgcctactcttgccaggcccaggatgcccagggcacccgcagctcccgtcctgctgccctgcaagtcctctatgcccctcaggacactgtcctgtcctccttccgggactccagggccagatccatggctgtgatacagtgcactgtggacagtgagccacctgctgagctggccctatctcatgatggcaaggtgctggccacgagcagcggggtccacagcttggcatcagggacaggccatgtccaggtggcccgaaacgccctacggctgcaggtgcaagatgtgcctgcaggtgatgacacctatgtttgcacagcccaaaacttgctgggctcaatcagcaccatcgggcggttgcaggtagaaggtgcacgcgtggtggcagagcctggcctggacgtgcctgagggcgctgccctgaacctcagctgccgcctcctgggtggccctgggcctgtgggcaactccacctttgcatggttctggaatgaccggcggctgcacgcggagcctgtgcccactctcgccttcacccacgtggctcgtgctcaagctgggatgtaccactgcctggctgagctccccactggggctgctgcctctgctccagtcatgctccgtgtgctctaccctcccaagacgcccaccatgatggtcttcgtggagcctgagggtggcctccggggcatcctggattgccgagtggacagcgagccgctcgccagcctgactctccaccttggcagtcgactggtggcctccagtcagccccagggtgctcctgcagagccacacatccatgtcctggcttcccccaatgccctgagggtggacatcgaggcgctgaggcccagcgaccaaggggaatacatctgttctgcctcaaatgtcctgggctctgcctctacctccacctactttggggtcagagccctgcaccgcctgcatcagttccagcagctgctctgggtcctgggactgctggtgggcctcctgctcctgctgttgggcctgggggcctgctacacctggagaaggaggcgtgtttgtaagcagagcatgggcgagaattcggtggagatggcttttcagaaagagaccacgcagctcattgatcctgatgcagccacatgtgagacctcaacctgtgccccacccctgggctga

SEQ ID NO: 18 - DNA, 12321 bp, Homo sapiens
ggatccgggtgagagcccagtgctggacgagcccggggcccaggggtcccggccgaaatccctgctgtctttcaggtcaaacgtcataatccccgaaccccagaaaggccgaaaggcaaggcaaccccgaaagacgacgaagtcaacctcagggcgcaggagagggagggccagtgtgctgccgacgagggaggctggagccgcggggacgaggcgccccatacagcggcaagagggtggagggcaggagctcgccatcctgggtgaaagcggggcccagcgaaggggcccggccacaggaatctcggttccaccccgctactcccggctgtgactccagtttcgtccccagccgccgggaccgccccctcgccccgcccccagcgggcactcaggccgtaccactgtgccttcatgggggtggagatagatcgtgggctagtcctgccgaggagagaggggttcttcctcaaaaaatatgattatgtatagtattcgcatgattctagttaacttgtttcccttctgcctgctcggaccctctacctgccctacgaagggggcggagtgcgttcctgcctccccctgctcttccgcgtttggtgcgcgcctgcgcggtgcgtaggcggcggagcgtacttaagcttcgacgcaggaggcggggctgctcagtcctccaggcgtcggtgctcagcggtgttggaacttcgttgcttgcttgcctgtgcgcgcgtgcgcggacatggcctcaaacggtaggtaagggcgcgaggcgacggcggcggcgcacccggccgaggcctcccagctgggcttttcgttttcagtgggaccggggcggcgatcccgtgtgggattttttggcgcccctgtggcgggaagccgcggagaagagtaactggaggaggctggtgtcgccattttgtttcgctcctctggccctcgcgcgcggggcgggaagtcttttctttgcagtccgtttgcttggggtgggcgttgggagggacgcttcttaggggtttgaagcgtcaggtgagggtggaaaacgcccattctccgtggcctcgcctcccccaactcccggccccgcgctcgagcccgctttgtcgcagtgctgcatccgggcactcgcggcgcgcacgcgctctgcgggcccctcccccttcgcggcgcgggtaccccttccccgcctcgtgttggttcagctttctgtcgcgagacccttcgcggaagactcggcggcgcgcgtccggtgtgagccttgtccctcagtggtccttcgcgaatgggcgggaccgctccgttcccgcctgggttgccacgcggctgggggcggaggctcgggatcggggccgccctctagcttaacggtttggcggcggtggtcagggttcgaccaacggacttggggacggcccgagagtttttcccgcctaaatttctttcttttttttttttggagacacggtcttcctctgtcgcccaggctggagtgcagtggtgcgatctcagatcactgcaacgtccacctcctgggttcaagtgattcttctgcctcagcctcctgagtagttgggattataggcgcccgccaccacagcccggctaatttttgtgtttcttagtaggggcggggtttcaccatgttggtcaggctggtctcgaactgctgacctcaggtgatccgcccgcctgggcctcccgaagtgtcgggattacagacgtaagccaaccacgcctggtctaaatttcttttttctgagatagggacggagaagaacggccgcccgaggccacaccctctcctggtcctctttcccttttcctgcgggggaagcgccgcgtttcctgtgctgggaggatgagttgatcttgttcgtgtatcttaagtggggcttttcaaaagcgcttttgttgctttttgttcgctgggggaaggcagggtaggtgagaaaacggaaccatctggagtcccagggctggggactcgagtacctgttgactttcgcctcctaaggcgagcagttgcatgatgtctgtcatttggggtccaagggctcttttgattctctggctttgcactaaaaagcccacttcatctccgggattggtcaaagagtgaagaatggccttttttgagactttcttattctgtgggtctgggctgagagagcagaggccacaatctgaacactgttcgaattcaaacctgtgccactttggtagttctgcgacgttgggagagttagtctcttgactcctggcgataatggctttctcatagagtggatgaaacgaatgcgcgatgttttagagcaatcactgacatgaattgggcttgcagaaaattgtggatgtccaccaagaccttggtttttccaatggttaaggcttctgggactctgtagaaacttgcattttcttcactttttctttattgtaagaacacttggctgataacgccagtttgttcctcttctcaaccagtgtctcatggaggaattttatgccctttgttgcaacatggtcctatttttatttttttttttgagatggagtcttgctctgttgccaggctggagtgcaatggctcactgcaacctctgcctcccgggttcaagcgattctcctgcttcacctcccgagtagctgggattacaggcgtgcgccaccatgcccggctaattttttgtatttttattagagatggggcttcaccatgttggtcaggccagtctcgaattcctgacctcaagtgatccacccacctcggcctcccaaactgctgggattacaggcatgatccaccgtgcctggcctacgtggtcctttttattcatcagtgcttgagttaaggaatttagctttaattcaactctttcagagtggcagctgaagataatgtgattgtatttttcttttggagattatacccaacaagcaacccaaaggtgagtgctatttttgggcttccagagtttgtagagggcaagggtggtcacgccatgttttctgatcacgctggttttccttttatttagctatggggcctaccccacccagcccgggcagggctattcccagcagagcagtcagccctacggacagcagagttacagtggttatagccagtccacggacacttcaggctatggccagagcagctattcttcttatggccagagccagaacagtgagtctttctcagcgggtcacctcttcctactctttctgaatattgcttttctttttcttgttttttggagacggagtctggtcctgttgcccaggctggagtgcagtggtgctgtctcagctcactgcaacatcagcctaccgggttcaaacgattctcctgcctcagcctcctgagtagctgggattacaggtacctgctaccacgcctggctaattttgtgtttttagtagagatggggtttcaccgtgttggacaggctggtctggaactcctgacctcctgcctgccttgacctgccaaagtgctgggattacaggcgtcagccacaatgccctgaatgttgcttttcttaaacctgagcagcactgagatgttgaaactgttccatatttcttttccgtgaaacagtgtataagtcttaaaactttttgggatctgagtcctttacagggcattgtggcacacctgtagtcccagctactgaggaagctgaggcgggaggatcccttgaattcaggagtttggggctgcagtgagctatgatggtgcctgtgaacagccactgcattccagcctgggcagtgtgttgaggccccatctcaaaaacataaaaaaaaaaacaaaaaacaaaaatgtttattggtttgtgattctgtttccatttattttctttggcttttaatttttttgactcttcttattttccatcagcatgaaagagagcatattttctaaaggaagaaccagttttaggccaattctgaaatggagaaaatggttttgtttgaaaatgtatgaaatcatgtgatacataaggaggtgggatttgccccaaggtcctgaagtgtaactaagaaaggtggttgtcctgtagatactgcacgcacagctgcatattacagtgctgttaacagggatccttgggcctgggtttagagggtggtgctggaaatggtgttgggattggcggggtgaaattggaactgtactaaagagttggtaaaagttgaagcattaaatttaggctttgaaaggagggtaactatctttgcctatgagttgcaacatcactaacagcttctgagaggctggctttatgagtataggtattatgttttctttaacccattccttacattttctctttcctggtggcttttgtgactccctttttcttatcctggtagcaggctatggaactcagtcaactccccagggatatggctcgactggcggctatggcagtagccagagctcccaatcgtcttacgggcagcagtcctcctaccctggctatggccagcagccagctcccagcagcacctcgggaaggtacggtggtgttgatgtcggggaaggcttgaaaagaggggtgaattgatgaggaatgataaagggaccagcagtaggagcagttcagaggtgtaattggggtaggggagcctgtgttgggtacagagaatggactccactaaaagtgaaaggaaattgggggctatgctgggattgtgattgtgttttttgtttgttttccctagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtgagatgtcttcagctttgtctgcagcccattttctttttctttttttttttttttttgagacggagtcttgctctgtctctgttgctgaggctggagtgcagtggcacaatctcggctcactgcaagctccgccttccgggttcgcgccagtctcctgcctcagcctcccgagtagctgggactacaggcatccgccaccacgcccggctaattttttgtatttttagtagagacggggtttcaccatgttagccaggatggtttcgatctcctgaccttgtgatccgcccgccttggcctcccaaagtgctgggattacaggcgtgagccactgtgcctggtgtctgcagcccattttctataaggatttgtattctcctgtttcagcttaaaagagggttcctgtcttgtttcctagctgtcttttgtccttcattgcctggcacttgtcaaaccttttcaaaccttttagtgctactttacaatctttttgttttttttttttaatcattctttcttttctcacaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctagaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcattttctgaagtgtggagttgtctctgtggagactcaagttacagatcttaaggggcctgcctagaattttctcctctgggcaggcgacccaggaaagggtttggagtgaggctgtgagcacttacttgatattttacaagttttggatttggtgttaatttttttccttgtccgttttttcctgttgactaacggctcatcttttccttgtttttgttttttttttgttctttttttccatgtcactaaaggccctcgggaccaaggatcacgtcatgactccggtgagttcacacgtggtggcatgaaaagagtggctaaagtggtatcaagactgcctggatgttctttgaaactattataaaaaggaaactgaaaaaaatggggatagagaaggaagggagttaggtgtgtccttagttagcagtgagaagtatttgttacgaagtatttctcagaaatacctggcttgtgggttccacccccagtgatttaggtctgagaggaccctgaaaatctacctttctaacaagtccccagtgatgctgatgcgtctggaccacactcagatggtttacagcagtggttctttcaaaatgtggatcatgtccaagttggtgaacaaaactagtgaaagacctgaccacatgaagtaaagcattgaactcctgtttaggttgtattgatgtttgtgtactagatttgaatgtaaaatgggtttcttatttaattcggggaccttcaactgaaagttgataaatactgatggacttttctgtgtggtcttgttgggcatttcactcctgagccctggtttccatactgtatactgggtgttaacatgtttcaaaggataattgtcaaactgaatctgaaatttatcagcatggctggcatatagggactcaaaagggatgtggatttctttttagttgtcttccataaaccaaatgataccagttgcttgatggatactaggtgctttaggttttttcctgtgttttttattttaccttttcacatttgcattttctctgttcaacaagcagaacaggataattcagacaacaacaccatctttgtgcaaggcctgggtgagaatgttacaattgagtctgtggctgattacttcaagcagattggtattattaaggtacttgtggagaggagtgggagctttctgtcagtgttgtaggcttgtggatttcacacattagtaaaagcaagtctttaatggttgccagcagtaaaaacaagtcttagtggttgttgccagcttaatttgttgaggaaagagccttagttactgttttctaaaagagaagttctatcttaacacaaaaagtataacttatcagagtaccctaaactcttgagatttgtactctatagtaacttttagttttatctttcaatattggagtgagagacagttttctttaatggaggtttacatgtgaggtaggaagaagtaactgggaagaggggagctgaagtttgggaattataaacctcatgttctagaggaagaagatggaaagggagtactgtagcctttaaaattgatgttacctcattttgctttcttcagacaaacaagaaaacgggacagcccatgattaatttgtacacagacagggaaactggcaagctgaagggagaggcaacggtctcttttgatgacccaccttcagctaaagcagctattgactggtttgatggtatgtatgagaaggctggcagaggtggggctggggatatagggcagcaagccttaggaaacaagccatagtttgagggttcttttgagtcttccaacacttactttagctgcggtttcaggtagtctcatttttgcttatgtgtcagcagattataaaccatttgagaaaggcacgcttctcttgtattttcggattaatgtgtcttgcatttaaagtctgttgatgattttttgtttctctaggtaaagaattctccggaaatcctatcaaggtctcatttgctactcgccgggcagactttaatcggggtggtggcaatggtcgtggaggccgagggcgaggaggtgaggagctacctgctagtggtgcagaggggtaatggggagagtgcagaagatggtaaaggcttgcatggaatgggttagatttaccaaacttggagagggagcagacccatacttggtctatctgcattaggacccatgggccgtggaggctatggaggtggtggcagtggtggtggtggccgaggaggatttcccagtggaggtggtggcggtggaggacagcagcgagctggtgactggaagtgtcctaatccgtgagtgaaacttaatttttttcttagttctcttgcatgcgtgctctttgatatattggtactgaggtatgtgcgtgttttccaaagaagtaaatgtcaaggccacactgttggggtcagatttagccaaaagcttacctaggtaaggttgatgtaatgggaaaggtaatggattgggttcagtaatactgatttttgttcctgactctgagaagcaagccgttttgtctttctgaagcttcagtttcctcactgtatctctaaagtcaccgtagtttcttcctagttctaggtcttgcctattccccatcgctccagactgattgtcttcctttctccttagcacctgtgagaatatgaacttctcttggaggaatgaatgcaaccagtgtaaggcccctaaaccagatggcccaggagggggaccaggtggctctcacatgggtaagaaaggcagacctggtgctagggagctgggaccaaagaatccttaatttttcagcggggaggctcggggaacataggggaatgggaatatgatagatcttgtttcttttgtcctagggggtaactacggggatgatcgtcgtggtggyagaggaggctatgatcgaggcggctaccggggccgcggcggggaccgtggaggcttccgagggggccggggtggtggggacagaggtggctttggccctggcaagatggattccaggtaagactttaaatcagaataaaaaagtagagcagttgaacagaggccataggataacagggttttgttgagaaagtggtttcattttgagggctaggtggaaagacctgaggttgtaaccagtagtggagagggaaggaaaattaactcagggggagtgaatctgtagacccacttgagataagatactcgctgggttaggtaggaggggcagataggatatctaggcttggagaggctggtaactcaaatataatggatacttaatttttttttttttttttgcaggggtgagcacagacaggatcgcagggagaggccgtattaattagcctggctccccaggttctggaacagctttttgtcctgtacccagtgttaccctcgttattttgtaaccttccaattcctgatcacccaagggttttttttgtgtcggactatgtaattgtaactatacctctggttcccattaaaagtgaccattttagttaaattttgttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaattgagaaa

SEQ ID NO: 19 - DNA, 1806 bp, Homo sapiens
ccttcgtggccgcagcctgcactctcagaaatcagacttgagtggccggaacccttgagaccagaggcttaccatgctgctccctaggagggccaggaactgctgacgtgaccactggacagttattcgtgtctcttacaattaccaaacagaatggacaagcttaataaaataaccgtccccgccagtcagaagttgaggcagcttcaaaagatggtccatgatattaaaaacaatgaaggtggaataatgaataaaatcaaaaagctaaaagtcaaagcacctccaagtgttcctcgaagggactacgcttcagagagccccgctgacgaagaggagcagtggtccgatgactttgacagcgactatgaaaatccagatgagcactcggactcagagatgtacgtgatgcccgccgaggagaacgctgatgacagctacgagccgcctccagtagagcaggaaaccaggccggttcacccagccctgcccttcgccagaggcgagtatatagacaatcgatcaagccagaggcattccccacccttcagcaagacacttcccagtaagcccagctggccttcagagaaagcaaggctcacctccaccctgccggccctgactgctttgcagaaacctcaagtcccacccaaacccaaaggcctccttgaggatgaggctgattatgtggtccccgtggaagataatgatgaaaactatattcatcccacagaaagcagttcacctccacctgaaaaagctcccatggtgaatagatcaaccaagccaaattcctcaacgcccgcctctcctccaggaacagcttcaggtcgaaacagtggggcctgggaaaccaagtcacctccaccagctgcaccatccccgttgccacgggccgggaaaaaaccaacgacaccactgaagacaactccagttgcctctcaacagaatgcttcaagtgtttgtgaagaaaaacctatacctgctgaacgccaccgagggtcaagtcacagacaagaagctgtgcagtcaccagtgtttcctcctgcccagaaacaaatccaccaaaaacccatacctctgccaagatttacagaagggggaaacccaactgtggatgggcccctacccagcttttcatctaattccactatttcagaacaggaagctggcgttctctgcaagccatggtatgctggagcctgtgatcgaaagtctgctgaagaggcattgcacagatcaaacaaggatggatcatttcttattcggaaaagctctggccatgattccaaacaaccatatacactagttgtattctttaataagcgagtatataatattcctgtgcgatttattgaagcaacaaaacaatatgccttgggcagaaagaaaaatggtgaagagtactttggaagtgttgctgaaatcatcaggaatcatcaacatagtcctttggttcttattgacagtcagaataacacaaaagattccaccagactgaagtatgcagttaaagtttcataaagggggaaaaaaaagatcaataccattgcttcagacactttcccaaagtttctccttttgagaaaaagtcccaaaacttcatattttggattatgaatcatccagtaataaaatggaagatggagtcagctattgaagtggtcatccatttctttttaagaagctcatgtggacttgttctattgcctgacctgatgaactgttaatatctggtgaggttgagttatcatgctactaatattttccaaataaatatttttatttttaaaaaaaaaaaaaaaa

SEQ ID NO: 20 - DNA, 8404 bp, Homo sapiens
cgttgctgtcgggcgggcgcgccaagacgtgggcacctcctcacccggaccccgggccccgccgagccgcctcctggctcccgtggccgccagcccgccccggccgcaccgagcgtcgggatccgaggtgggagcgtacccctccccgctccccggacgcctcagtcctccgcactgagcttggccacgcgcccctaggcgccccccacgccctgggccccggagggccgcagccatgagtgaaatgtccagctttcttcacatcggggacatcgtctccctgtacgccgagggctccgtcaatggcttcatcagcactttggggctggtggatgaccgctgtgtggtggagcccgcggccggggacctggacaacccccctaagaagttccgtgactgcctcttcaaggtgtgccccatgaaccgctactcggcccagaagcagtactggaaggccaagcagactaagcaggacaaggagaagatcgctgatgtggtgttgctgcagaagctgcagcatgcggcgcagatggagcagaagcaaaatgacacggagaacaagaaggtgcatggggatgtcgtgaagtatggcagtgtgatccagctcctgcacatgaagagcaacaagtacctgacagtgaacaagcggcttccggccttgctggagaagaacgccatgcgggtgactctggatgccacaggcaacgagggttcctggctcttcatccagcccttctggaagctgcggagcaacggggacaacgtggtcgtgggggacaaggtgatcctgaatcctgtcaatgccgggcagcctctgcatgccagcaattacgagctcagcgacaacgccggctgcaaggaggtcaattctgtgaactgcaacaccagctggaagatcaacctgtttatgcagtttcgggaccacctggaggaggtgttgaaagggggagacgtggtgcggctgttccatgcggagcaggagaagttcctgacgtgtgacgagtacaagggcaagctgcaggtgttcctgcgaactacactgcgccagtctgccacctcggccaccagctccaatgctctctgggaggtggaggtggtccaccacgacccctgccgtggaggagctgggcactggaatggcttgtaccgcttcaagcacctggctacaggcaactacctggctgctgaggagaaccccagttacaaaggtgatgcctcagatcccaaggcagcaggaatgggggcacagggccgcacaggccgcaggaatgctggggagaagatcaagtactgcctggtggctgtgcctcatggcaatgacatcgcctctctctttgagctggaccccaccaccttgcagaaaaccgactctttcgtgccccggaactcgtacgtccggctgcggcacctctgcaccaacacgtggattcagagcaccaatgtgcccattgacatcgaggaggagcggcccatccggctcatgctgggcacctgccccaccaaggaggacaaggaggcctttgccatcgtgtcagtgcccgtgtctgagatccgagacctggactttgccaatgacgccagctccatgctggccagtgccgtggagaaactcaacgagggcttcatcagccagaatgaccgcaggtttgtcatccagctgctggaagacctggtgttctttgtcagcgatgtccccaacaatgggcagaatgtcctggacatcatggtcactaagcccaaccgggaacggcagaagctgatgagggagcagaacatcctcaaacaggtctttggcattctgaaggccccgttccgtgagaaggggggtgaaggtcccctggtgcggctggaggagctgtcagaccagaagaacgccccctaccagcacatgttccgcctgtgctaccgcgtgttgcggcattcccaggaggactaccgcaagaaccaggagcacattgccaagcagtttgggatgatgcagtcccagattggctacgacatcctggccgaggacaccatcactgccctgctgcacaacaaccgcaagctcctggaaaagcacatcaccaagaccgaggtggagaccttcgtcagccttgtgcgcaagaaccgggagcccaggttcctggactacctctctgacctgtgtgtgtccaaccacatcgccatccccgtcacccaagagctcatctgcaagtgtgtgctggaccccaagaacagtgacattctcatccggaccgagcttcggcccgtgaaggagatggcccaatcccacgagtacctgagcatcgagtactcagaagaggaagtgtggctcacgtggactgacaagaataacgagcatcatgagaagagtgtgaggcagctggcccaggaggcgcgggccggcaacgcccatgacgagaatgtgctcagctactacaggtaccagctgaagctctttgcccgcatgtgcttggaccgccagtacttggccatcgacgagatctcccagcagctgggtgtggacctgattttcctgtgcatggcagacgagatgctgccctttgacctgcgcgcctccttctgccacctgatgctgcacgtgcacgtggaccgtgacccccaggagctggtcacgccggtcaagtttgcccgtctctggactgagatccccacagccatcaccatcaaggactatgattccaacctcaacgcgtcccgagatgacaagaagaacaagtttgccaacaccatggagttcgtggaggactacctcaacaatgtagtcagcgaggccgtgccctttgccaacgaggagaagaacaagctcacttttgaggtggtcagcctggcgcacaatctcatctacttcggcttctacagcttcagcgagctgctgcggctcactcgcacactgctgggcatcatcgactgtgtgcaggggcccccggccatgctgcaggcctatgaggaccccggtggcaagaatgtgcggcggtccatccagggcgtggggcacatgatgtccaccatggtgctgagccgcaagcagtccgtcttcagtgcccccagcctgtctgctggggccagtgctgctgagccgctggacagaagcaagtttgaggagaatgaggacattgtggtgatggagaccaagctgaagatcctggaaatccttcagttcatcctcaacgtccgcctggattaccgcatatcccacctgctgtctgtcttcaagaaggagtttgtggaggtgtttcccatgcaggacagtggggctgatggcacagcccctgccttcgactctaccactgccaacatgaacctggatcgcatcggggagcaggcggaggccatgtttggagtggggaagacaagcagcatgctggaggtggatgacgagggcggccgcatgttcctgcgcgtgctcatccacctcaccatgcacgactatgcgccgctggtctcgggtgccctgcagctgctcttcaagcacttcagccagcgccaggaggccatgcacaccttcaagcaggttcagctgctgatctcagcgcaggacgtggagaactacaaggtgatcaagtcggagctggaccggctgcggaccatggtggagaagtcagagctgtgggtggacaagaagggcagtggcaagggtgaggaggtggaggcaggcgccgccaaggacaagaaagagcgtcccacggacgaggagggctttctgcacccaccaggggagaaaagcagtgagaactaccagatcgtcaagggcatcctggaaaggctgaacaagatgtgcggggttggggagcaaatgaggaagaagcagcaacggctgctgaagaacatggatgcccacaaggtcatgctggacctgctgcagatcccctatgacaagggtgatgccaagatgatggagatcctgcgctacacgcaccagttcctgcagaagttctgtgcagggaaccccggcaaccaggccctgctgcacaaacacctgcacctcttcctcacgccagggctcctggaggcagagaccatgcagcacatcttcctgaacaactatcagctctgctccgagatcagcgagcctgtgttgcagcacttcgtgcacctgctggccacgcacgggcgccatgtgcagtacctggacttcctgcacaccgtcattaaggccgagggcaagtacgtcaagaagtgccaggacatgatcatgactgagctgaccaatgcaggtgacgatgtggtcgtgttctacaatgataaggcatcgctggcccacctgctggacatgatgaaggccgcccgcgacggcgtggaggaccacagccccctcatgtaccacatttccctggtggacctgctggccgcctgtgccgagggcaaaaacgtctacactgagatcaagtgcacctccctgctgccgctggaggacgtggtgtctgtggtgacgcatgaggactgcatcactgaggtgaaaatggcctatgtgaacttcgtgaaccactgctacgtggacacggaggtggagatgaaggagatctacaccagcaaccacatctggacgctctttgagaacttcaccctggacatggctcgggtctgcagcaagcgtgagaagcgcgtggctgaccccaccttggagaagtacgtgctgagcgttgtgctggacaccatcaacgccttcttcagctccccattctctgagaacagcacttccctgcagacacaccagacgattgtggtgcagctgctgcagtctaccacacgcctcctcgagtgtccgtggctacagcagcagcacaagggctccgtggaggcctgcatccggaccctcgccatggtggccaagggccgggccatcttgctgcccatggacctggatgcccacatcagctcgatgctcagcagtggagccagctgtgcagctgccgcccagcggaacgcctccagctacaaggcaaccacgcgggccttcccccgcgtcacccctaccgccaaccagtgggactacaagaacatcattgagaagctgcaggacatcatcacagccctggaggagcggctgaagcccctggtacaggctgagctgtccgtgctggtggatgtcctgcactggcctgagctgctcttcctggagggcagtgaggcctaccagcgctgcgagagtgggggcttcctgtccaagctgatccagcacaccaaggacctcatggagtcggaggagaagctgtgcatcaaggtgctgcggaccctgcagcagatgctgctcaagaagaccaagtacggggaccggggcaaccagctgcgcaagatgctgctgcaaaactacctccagaaccggaagtccacctcgcggggggaccttcccgaccccataggcactggcctggacccagactggtcggcaatcgcagccacccagtgccggctggacaaggagggggccaccaagttggtatgcgacctcatcaccagcaccaagaacgagaagatcttccaggagagcatcggcctggccatccacctgctggatggtggcaacacagagatccagaaatccttccacaacctgatgatgagtgacaagaagtcagagcgcttcttcaaggtgctgcacgaccgcatgaagcgggcccagcaggagaccaagtccacggtggcagtcaacatgaatgacctgggcagccagccacatgaggaccgcgagccagtcgaccccaccaccaaaggccgcgtggcctccttctcgatacctggttcctcatcccgctactcgctgggccccagcctgcgccgggggcacgaggtgagcgaacgtgtgcagagcagtgagatgggcacatccgtgctcatcatgcagcccatcctgcgctttctgcagctgctgtgtgagaaccacaaccgggacctgcagaacttcctgcgctgtcagaacaacaaaaccaactacaacttggtatgcgagacgctgcagttcctggacatcatgtgcggcagcaccacgggcggcctggggctgctggggctctacatcaatgaggacaacgtgggcctcgtcatccagaccttggagaccctcactgagtactgccagggcccctgccatgagaaccagacttgcattgtgactcacgagtccaatggcatagacatcatcaccgcactgatcctcaatgacatcagccccctgtgcaagtaccgcatggatctggtgctgcagctcaaggacaatgcctccaagctgctcctggctctgatggagagccggcatgacagtgaaaatgctgagcgaatcctcatcagcctgcggccccaggagctggtggacgtcatcaagaaggcctacctgcaggaggaagagcgtgagaactcggaggtgagcccacgtgaagtgggccataacatctatatcctggcgctgcagctctccaggcacaataaacagctgcagcacctgctgaagccggtgaagcgcattcaagaggaggaggccgagggtatctcttccatgctcagcctcaacaacaagcagctgtcacagatgctcaagtcctcagcgccagcacaggaggaggaggaagaccccctggcctactatgagaaccacacgtcccagatcgagattgtgcggcaggaccgcagcatggagcagatcgtgttcccagtgcccggcatctgccagttcctgacggaggaaaccaagcaccggctcttcaccactactgagcaggacgagcagggcagcaaagtgagcgacttcttcgaccagtcctccttcctgcacaacgagatggagtggcagcgcaacgtccgcagcatgccgctgatctactggttctcccgccgcatgaccctgtggggcagcatctccttcaacctggccgtgtttatcaacatcatcattgccttcttctacccttacatggagggcgcgtccacaggcgtgctggactcccctctcatctcattgctcttctggatcctcatctgcttctccatcgcggccctgttcaccaagcgctacagcatccgccccctcatcgtggcgctcatcctgcgctccatctactatctgggcatcgggcccacactcaacatcctgggtgccctcaatctgaccaacaagatcgtgtttgtggtgagcttcgtgggcaaccgtggcaccttcatccggggctataaggccatggtcatggacatggaattcctctaccacgtgggctacatcctgaccagtgtcctgggcctctttgctcatgagctgttctacagcatcctgctctttgacctcatctaccgcgaggagacgctgttcaacgtcatcaagagtgtgacccgcaatggccgctccatcctgctgacagccctgctggccctcatcctggtctacctcttctccatcgtcggcttcctcttcctcaaggatgacttcattctcgaggtcgaccggctgcccaacaaccactccacagccagccccctggggatgccacatggagctgctgcatttgtggacacctgcagtggggacaagatggactgtgtctcaggggtctcggtgcctgaggtcctggaagaggacagggagctggacagcacagagcgggcctgtgacactctgttgatgtgcatcgtcactgtcatgaaccatgggctacgcaacggtggtggcgtgggcgacattctccgcaagccctccaaagatgagtctctcttcccagcccgagtggtctatgacctcctgttcttcttcatcgtcatcatcattgtgctgaacctcatctttggggtaatcatcgacaccttcgctgacctgcgtagtgagaagcagaagaaggaggagattcttaagacgacatgcttcatctgtggtctggagagggacaagtttgataacaagacagtgtcatttgaggaacacatcaagctggagcacaacatgtggaactacttgtacttcattgtgctggtccgcgtgaagaacaagaccgactacacgggccctgagagctacgtggcccagatgatcaagaacaagaacctggactggttcccccggatgcgggccatgtcccttgtcagcaatgagggcgagggggagcagaatgagattcggattctccaggacaagctcaactccaccatgaagctggtgtcccacctcactgcccagctcaacgagctcaaggagcagatgacggagcagcggaaacgcaggcaacgcctaggctttgtggatgtccagaactgcattagccgctgaggagagccaccgaaggccccaacaggggatgctcatcactggagactgcgactgggaagaacactgccccctccctcgggttgggtggcccagccagctggccagcctccactcccactctgccagacaccctgacacccacccaggctttcg

SEQ ID NO: 21 - DNA, 2457 bp, Homo sapiens
acgcggagaaaatggcggcaggggtcgaagcggcggcggaggtggcggcgacggagatcaaaatggaggaagagagcggcggcccggcgtgcgagcggcaacggggctccgggcctaagggtgaaggagaacgacctgctcagaatgagaagaggaaggtgaaaaacataaaaagaggaggcaatcgctttgagccatatgccaatccaactaaaagatacagagccttcattacaaacataccttttgatgtgaaatggcagtcacttaaagacctggttaaagaaaaagttggtgaggtaacatacgtggagctcttaatggacgctgaaggaaagtcaaggggatgtgctgttgttgaattcaagatggaagagagcatgaaaaaagctgcggaagtcctaaacaagcatagtctgagcggaagaccactgaaagtcaaagaagatcctgatggtgaacatgccaggagagcaatgcaaaaggtgatggctacgactggtgggatgggtatgggaccaggtggcccaggaatgattactatcccacccagtatcctaaataatcccaacatcccaaatgagattatccatgcattacaggctggaagacttggaagcacagtatttgtagcaaatctggattataaagttggctggaagaaactgaaggaagtatttagtatggctggtgtggtggtccgagcagacattcttgaagataaagatggaaaaagtcgtggaataggcactgttacttttgaacagtccattgaagctgtgcaagctatatctatgttcaatggccagctgctatttgatagaccaatgcacgtcaagatggatgagagggccttaccaaaaggagatttcttccctcctgagcgtccacaacaacttccccatggccttggtggtattggcatggggttaggaccaggagggcaacccattgatgccaatcacctgaataaaggcatcggaatgggaaacataggtcccgcaggaatgggaatggaaggcataggatttggaataaataaaatgggaggaatggaggggccctttggtggtggtatggaaaacatgggtcgatttggatctgggatgaacatgggcaggataaatgaaatcctaagtaatgcactgaagagaggagagatcattgcaaagcagggaggaggtggaggtggaggaagcgtccctgggatcgagaggatgggtcctggcattgaccgcctcgggggtgccggcatggagcgcatgggcgcgggcctgggccacggcatggatcgcgtgggctccgagatcgagcgcatgggcctggtcatggaccgcatgggctccgtggagcgcatgggctccggcattgagcgcatgggcccgctgggcctcgaccacatggcctccagcattgagcgcatgggccagaccatggagcgcattggctctggcgtggagcgcatgggtgccggcatgggcttcggccttgagcgcatggccgctcccatcgaccgtgtgggccagaccattgagcgcatgggctctggcgtggagcgcatgggccctgccatcgagcgcatgggcccgagcatggagcgcatggtgcccgcaggtatgggagctggcctggagcgcatgggccccgtgatggatcgcatggccaccggcctggagcgcatgggcgccaacaatctggagcggatgggcctggagcgcatgggcgccaacagcctcgagcgcatgggcctggagcgcatgggtgccaacagcctcgagcgcatgggccccgccatgggcccggccctgggcgctggcattgagcgcatgggcctggccatgggtggcggtggcggtgccagctttgaccgtgccatcgagatggagcgtggcaacttcggaggaagcttcgcaggttcctttggtggagctggaggccatgctcctggggtggccaggaaggcctgccagatatttgtgagaaatctgccattcgatttcacatggaagatgctaaaggacaaattcaacgagtgcggccacgtgctgtacgccgacatcaagatggagaatgggaagtccaaggggtgtggtgtggttaagttcgagtcgccagaggtggccgagagagcctgccggatgatgaatggcatgaagctgagtggccgagagattgacgttcgaattgatagaaacgcttaagcagttgccttttttaaacatcgatacgagacctctgaatttgtattttttcttgttaaccattttaatttgttggctggatgtataaagatgtttaaaaaattcagttgctttttggggtaatttgaattacttttttaatgactggggttccatttgactgtttgcattgagattgcaatgtgcgcaattttttttgtagttgtggcatcttgttgacatcgaatatgactttgataataaataccggttcctg

SEQ ID NO: 22 - DNA, 573 bp, Homo sapiens
ctgactgaggggtcagtggttccgggtaggagctaggtgaccctcggctgctgcagggatctgcagcgactgcagccatgggggcccacctggtccggcgctacctgggcgatgcctcggtggagcccgaccccctgcagatgccaaccttcccgccagactacggcttccccgaacgcaaggagcgcgagatggtggccacacagcaggagatgatggacgcgcagctgaggctccagctgcgggactactgcgcccaccacctcatccggctgctcaagtgcaagcgtgacagcttccccaacttcctggcctgcaagcaggagcggcacgactgggactactgcgagcaccgcgactatgtgatgcgcatgaaggagtttgagcgggagcggaggctgctccagcggaagaagcggcgggagaagaaggcggcagagttggccaaaggccagggacccggggaagtggaccccaaggtggccctgtagggggtgcaccccccaccctatggaccagtcaaataaaagccttcaggcccctcaaaaaaaaaaaaaaaaaaaaaaaaaaaaa

SEQ ID NO: 23 - DNA, 2613 bp, Homo sapiens
cttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaagtagacaagaggaacctgctgtcaagtccctgccattttgcctctcctgtctcccaccccctgccccagacccaggagcccccctgaggctttgccttgcctgcatatttgtttcgctcttactcagtttgggaattc

SEQ ID NO: 24 - DNA, 745 bp, Homo sapiens
ccaatgactttgtcatgctgaaaggctgtgtggtgggaaccaagaagcgggtgctcaccctccgcaaggtgaggcaggtcctgacttgtcatgggggggctcttgtagagggaggatccaaggccacagacgctggtggccctgtggtgggagaggaaaggaaggagagggtgttggcagtcctttcacactggctttgaagtcctgagatgaggaaattcccagtctggccttgctgggctgtttgctgctttgagtgtgtcctcatctgccggatggtgggggaggctgaattgatcgtgtggcttcccagatgcaggggcgctgtgaacagtgctgaggaaagcctggctcctcgtaatcctaaatcccttcccctcttggcccatagtccttgctggtgcagacgaagcggcgggctctggagaagattgaccttaagttcattgacaccacctccaagtttggccatggccgcttccagaccatggaggagaagaaagcattcatggtgagcacctctgcctgcccccctcgctgggtgtggtggcttgggcagccgcctgcctttggctccagccggggtaggggtggtcttgatgccttaggcctctcttgtggctgtggagactggcccagagtcttggtctcctctagagtgggcatctgctgtcacctgtcatcttctacctctgactactgcctctctccccacagggaccactgaagaaagaccgaattgcaa

SEQ ID NO: 25 - DNA, 4034 bp, Homo sapiens
gcagccagagctcagcagggccctggagagatggccacggtcccagcaccggggaggactggagagcgcgcgctgccaccgccccatgtctcagccagggcttccttcctcggctccaccctgtggatgtaatggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacaccattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtagacagatggccagggtgggaggcaggcagctgcctgctctgcgccgagcctcagaaggaccctgttgagggtcctcagtccactgctgaggacactcagtgtccagttgcagctggacttctccacccggatggcccccacccagtcctgcacacttggtccatccatttccaaacctccactgctgctcccgggtcctgctgcccgagccaggaactgtgtgtgttgcaggggggcagtaactccccaactccctcgttaatcacaggatcccacgaatttaggctcagaagcatcgctcctctccagccctgcagctattcaccaatatcagtcctcgcggctctccagggctccctgccctgacctcttccctgggttttctgccccagcctcctccttccctcccctccccgtccacagggcagcctgagcgtgctttccaaaacccaaatatggccacgctccccctcggttcaaaaccttgcacaggtcccactgccctcagccccacttctcagcctggtacttgtacctccggtgtcgtgtggggacatccccttctgcaatcctccctaccgtcctcccgagccactcagagctccctcacaccccctctgttgcacatgctattccctggggctgctgtgcgctccccctcatctaggtgacaaacttccctgactcttcaagtgccggttttgcttctcctggagggaagcactgcctcccttaatctgccagaaacttctagcgtcagtgctggagggagaagctgtcagggacccagggcgcctggagaaagaggccctgttactattcctttgggatctctgaggcctcagagtgcttggctgctgtatctttaatgctggggcccaagtaagggcacagatccccccacaaagtggatgcctgctgcatcttcccacagtggcttcacagacccacaagagaagctgatggggagtaaaccctggagtccgaggcccaggcagcagccccgcctagtggtgggccctgatgctgccaggcctgggacctcccactgccccctccactggaggggtctcctctgcagctcagggactggcacactggcctccagaagggcagctccacagggcagggcctcattatttttcactgccccagacacagtgcccaacaccccgtcgtataccctggatgaacgaattaattacctggcaccacctcgtctgggctccctgcgcctgacattcacacagagaggcagagtcccgtgcccattaggtctggcatgccccctcctgcaaggggctcaaccccctaccccgacccctccacgtatctttcctaggcagatcacgttgcaatggctcaaacaacattccaccccagcaggacagtgaccccagtcccagctaactctgacctgggagccctcaggcacctgcacttacaggccttgctcacagctgattgggcacctgaccacacgcccccacaggctctgaccagcagcctatgagggggtttggcaccaagctctgtccaatcaggtaggctgggcctgaactagccaatcagatcaactctgtcttgggcgtttgaactcagggagggaggcccttgggagcaggtgcttgtggacaaggctccacaagcgttgagccttggaaaggtagacaagcgttgagccactaagcagaggaccttgggttcccaatacaaaaatacctactgctgagagggctgctgaccatttggtcaggattcctgttgcctttatatccaaaataaactcccctttcttgaggttgtctgagtcttgggtctatgccttgaaaaaagctgaattattggacagtctcacctcctgccatagggtcctgaatgtttcagaccacaaggggctccacacctttgctgtgtgttctggggcaacctactaatcctctctgcaagtcggtctccttatccccccaaatggaaattgtatttgccttctccactttgggaggctcccacttcttgggagggttacattttttaagtcttaatcatttgtgacatatgtatctatacatccgtatcttttaatgatccgtgtgtaccatctttgtgattatttccttaatattttttctttaagtcagttcattttcgttgaaatacatttataaagaaaaatctttgttactctgtaaatgaaaaaacccattttcgctataaataaaaggtaactgtacaaaataagtacaat

SEQ ID NO: 26 - DNA, 1937 bp, Homo sapiens
ggctggggggcttagaattttatttttggtttacagcgcagaagaagagcagtgaggccggggcgcgggtggggagggcaaggcggcggctcaaaaaaaaagccagcccctcgcggcccgacagagtctcggcgccccgcgggtcggtggagcctcaggggtgagcgcaggccggagacaggggaacccgccgcgggacaggggaaggccggacggaacgcgggtaagctgccgcagcttccggccgggccactagcgcggaccctacggccggggcgggacttccggcaaggcgcggaagcggcggtagctgcagctggcgaagttgggcgactggcggatgcaggccttgcggcacgtcgtgtgcgccctgtccggcggcgtggacagcgccgtggccgcgctgctgctgaggcggagaggttaccaggtgacaggggtgtttatgaagaactgggactcactggatgaacatggggtctgtactgccgacaaagactgtgaagatgcttacagagtttgccagatcttagacatccctttccatcaagtgtcctacgtaaaggagtattggaatgatgtgttcagtgactttttgaatgagtatgaaaaaggaaggactcccaatcctgacatagtttgcaacaagcacatcaaatttagttgcttttttcattatgctgtggataatcttggggcagatgccattgccacaggtcactatgcaagaacttccctggaagatgaagaagtctttgagcagaagcacgttaagaagcccgaagggcttttcagaaatcggtttgaagttagaaatgcggtaaaactcctccaggcagctgacagctttaaagaccagaccttctttctcagccaggtttcccaggatgccctgaggagaaccatcttccctctggggggattaacgaaagagtttgtaaagaaaatcgctgctgagaatagacttcatcatgtgcttcagaagaaagagagcatgggcatgtgtttcatcgggaagaggaattttgaacatttccttcttcagtatctgcagcctcgacctggtcactttatttccatagaagacaataaggttctgggaacacataaaggttggttcctgtataccttgggccagagagcaaacataggtggcctgagagagccctggtacgtggtggagaaggacagcgtcaagggtgacgtgtttgtggccccccggacagaccacccagccctgtacagggacctgctgaggaccagccgcgtgcactggattgcggaggagcctcccgcagcactggtccgggacaagatgatggagtgccacttccgattccgccaccagatggcactagtgccctgtgtgctgaccctcaatcaagatggcaccgtgtgggtgacagctgtgcaggctgtgcgtgcccttgccacaggacagtttgctgtgttctacaagggggacgagtgcctgggcagcgggaagatcctgcggctggggccgtctgcctacacgctccagaagggccagcgcagagctgggatggccactgagagccccagtgacagcccagaagatggtccaggcctgagtcccttgctctgacagagatggatctgctagaaggaacctggagagcaggacccatggctgggcggctggtgagcagtccaggtgcccaagggccagcttgctgctgcccaaagcagaggaagccgggctggctgagggtccgaaaagcctgcaggggcccggcgagccccaggaagagcctcagctccaggctggggctctggctgctggagcatctgctggctggtggggtggcccgagttccccttcaccgcccccagggagggtttcccacctcagagtacaccgaggggacctgcagagggggctgtcgggacagcgtggaataaacattatttcaagg

SEQ ID NO: 27 - DNA, 1614 bp, Homo sapiens
ggaaggaagtgaaaatgggtgtccctgctgcctcttagcaacaagaggggtcaagtgacacaaccagctgactcccgtagaggaagacactgtggaggccagttctggagctattgcagcctcggttgcccggccggggacccgagccgaaaagttatcgtcagaatgtcgggcaaagaccgaattgaaatctttccctcgcgaatggcacagaccatcatgaaggctcgtttaaagggagcacagacaggtcgaaacctcctgaagaaaaaatctgatgccttaactcttcgatttcgacagatcctaaagaagataatagagactaaaatgttgatgggcgaagtgatgagagaagctgccttttcactagctgaagccaagttcacagcaggtgacttcagcactacagttatccaaaatgtcaataaagcgcaagtgaagattcgagcgaagaaagataatgtagcaggtgttactttgccagtatttgaacattaccatgaaggaactgacagttatgaactgactggtttagccagaggtggggaacagttggctaaattaaagaggaattatgccaaagcagtggaactactggtggaactagcttctctgcagacttcttttgttactttggatgaagctattaagataaccaacaggcgtgtaaatgccattgaacatgtcatcattccccggattgaacgtactcttgcttatatcatcacagagctggatgagagagagcgagaagagttctataggttaaagaaaatacaagagaagaaaaagattctaaaggaaaaatctgagaaggacttggagcaaaggagagcagctggagaggtgttggagcctgctaatcttctggctgaagagaaggacgaggatcttctatttgaataatctttcctgttctggttctttgagaaaccctaacactggcttcattttaattcacagtgtgtaggtttgatttgtgtggctattgattttttggcctaagaatttcactggttgtaaaatttacctagatgtctatttatgggattacttttgcagaatcataatttagcaaccatttatcatggatgaaagagatctgtaaaacctgcccaggaacttacagaatttactttgcagaagcgttatcatactccatttacatctgtgttacacgtgatctgcttaccaagcatattaggaaatacctcttaggaagcattagcggtctcaggccaattactgtggagcagctttcattcctacccacttgcaaaccttggcgctgttgtctgagattgctgcagccattcttgttaccatggtacttctcaaactttgtgaaaacctgcacttttccttgcatgacaggttcctgtcttgtctgtcatgggagccattctgccaatttaaatgcgactgtggtataaacagtaaaatgatttaaaagtaagtcattccgtttttattaatttactgttaagtcatgttctcatgctcagatcagtagtgtcagccagagctttctctgcagacatgtaggaagtgggtagctatttttcccactccatgtattagagttttacaaaaaggcttacttttgagaca

SEQ ID NO: 28 - DNA, 1850 bp, Homo sapiens
gaatcgcctgccacaggtgtctgcaattgaactccaaggtgcagaatggtttggaaagtagctgtattcctcagtgtggccctgggcattggtgccgttcctatagatgatcctgaagatggaggcaagcactgggcagtgatcgtggcaggttcaaatggctggtataattataggcaccaggcagacgcgtgccatgcctaccagatcattcaccgcaatgggattcctgacgaacagatcgttgtgatgatgtacgatgacattgcttactctgaagacaatcccactccaggaattgtgatcaacaggcccaatggcacagatgtctatcagggagtcccgaaggactacactggagaggatgttaccccacaaaatttccttgctgtgttgagaggcgatgcagaagcagtgaagggtataggatccggcaaagtcctgaagagtggcccccaggatcacgtgttcatttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgctgccaaccccagagagtcgtcctacgcctgttactatgatgagaagaggtccacgtacctgggggactggtacagcgtcaactggatggaagactcggacgtggaagatctgactaaagagaccctgcacaagcagtaccacctggtaaaatcgcacaccaacaccagccacgtcatgcagtatggaaacaaaacaatctccaccatgaaagtgatgcagtttcagggtatgaaacgcaaagccagttctcccgtccccctacctccagtcacacaccttgacctcacccccagccctgatgtgcctctcaccatcatgaaaaggaaactgatgaacaccaatgatctggaggagtccaggcagctcacggaggagatccagcggcatctggatgccaggcacctcattgagaagtcagtgcgtaagatcgtctccttgctggcagcgtccgaggctgaggtggagcagctcctgtccgagagagccccgctcacggggcacagctgctacccagaggccctgctgcacttccggacccactgcttcaactggcactcccccacgtacgagtatgcgttgagacatttgtacgtgctggtcaacctttgtgagaagccgtatccgcttcacaggataaaattgtccatggaccacgtgtgccttggtcactactgaagagctgcctcctggaagcttttccaagtgtgagcgccccaccgacatgtgtgctgatcagagactggagaggtggagtgagaagtctccgctgctcgggccctcctggggagcccccgctccagggctcgctccaggaccttcttcacaagatgacttgctcgctgttacctgcttccccagtcttttctgaaaaactacaaattagggtgggaaaagctctgtattgagaagggtcatatttgctttctaggaggtttgttgttttccctgttagttttgaggagcaggaagctcatgggggcttctgtagcccctctcaaaaggagtctttattctgagaatttgaagctgaaacctctttaaatcttcagaatgattttattgaagaggggcgcaagccccaaatggaaaactgtttttagaaaatatgatgatttttgattgcttttgtatttaattctgcaggtgttcaagtcttaaaaaataaagatttataacag

SEQ ID NO: 29 - DNA, 931 bp, Homo sapiens
tttcgtcggcccgccccttggcttctgcactgatggtgggtggatgagtaatgcatccaggaagcctggaggcctgtggtttccgcacccgctgccacccccgcccctagcgtggacatttatcctctagcgctcaggccctgccgccatcgccgcagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagttttgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaagctgaagcctgcacagtgtccaccctgttcccactcccatctttcttccggacaatgaaataaagagttaccacccagcagaaaaaaaaaaaaaaaa

SEQ ID NO: 30 - DNA, 2755 bp, Homo sapiens
ccgggccgcgcttcctctcgccaggcctgcgagcttcctcccagcggagccctgggcgagccgaggttggccgccgccgccgccgagcccgctgccgccctcccgctcctgccccacccgcgccttgcccgggggcttctgccggggtggggtccgagccgggcgaccgcccggctgcgccgccgtcggggccgtaacccggcccgccgtccctcccgccccagccagcctctggccgccggagcccgcggggcgtggagcgcgaggagccccgcggccccgatcgagcgtccggggcggcccccggcagccagcgcgacgttccaaaatcgaacctcagtggcggcgctcggaagcggaactctgccggggccgcgccggctacattgtttcctccccccgactccctcccgcccccttcccccgcctttcttccctccgcgacccgggccgtgcgtccgtccccctgcctctgcctggcggtccctcctcccctctccttgcacccatacctctttgtaccgcaccccctggggacccctgcgcccctcccctcccccctgaccgcatggaccgtcccgcaggccgctgatgccgcccgcggcgaggtggcccggaccgcagtgccccaagagagctctaatggtaccaagtgacaggttggctttactgtgactcggggacgccagagctcctgagaagatgtcagcaatacaggccgcctggccatccggtacagaatgtattgccaagtacaacttccacggcactgccgagcaggacctgcccttctgcaaaggagacgtgctcaccattgtggccgtcaccaaggaccccaactggtacaaagccaaaaacaaggtgggccgtgagggcatcatcccagccaactacgtccagaagcgggagggcgtgaaggcgggtaccaaactcagcctcatgccttggttccacggcaagatcacacgggagcaggctgagcggcttctgtacccgccggagacaggcctgttcctggtgcgggagagcaccaactaccccggagactacacgctgtgcgtgagctgcgacggcaaggtggagcactaccgcatcatgtaccatgccagcaagctcagcatcgacgaggaggtgtactttgagaacctcatgcagctggtggagcactacacctcagacgcagatggactctgtacgcgcctcattaaaccaaaggtcatggagggcacagtggcggcccaggatgagttctaccgcagcggctgggccctgaacatgaaggagctgaagctgctgcagaccatcgggaagggggagttcggagacgtgatgctgggcgattaccgagggaacaaagtcgccgtcaagtgcattaagaacgacgccactgcccaggccttcctggctgaagcctcagtcatgacgcaactgcggcatagcaacctggtgcagctcctgggcgtgatcgtggaggagaagggcgggctctacatcgtcactgagtacatggccaaggggagccttgtggactacctgcggtctaggggtcggtcagtgctgggcggagactgtctcctcaagttctcgctagatgtctgcgaggccatggaatacctggagggcaacaatttcgtgcatcgagacctggctgcccgcaatgtgctggtgtctgaggacaacgtggccaaggtcagcgactttggtctcaccaaggaggcgtccagcacccaggacacgggcaagctgccagtcaagtggacagcccctgaggccctgagagagaagaaattctccactaagtctgacgtgtggagtttcggaatccttctctgggaaatctactcctttgggcgagtgccttatccaagaattcccctgaaggacgtcgtccctcgggtggagaagggctacaagatggatgcccccgacggctgcccgcccgcagtctatgaagtcatgaagaactgctggcacctggacgccgccatgcggccctccttcctacagctccgagagcagcttgagcacatcaaaacccacgagctgcacctgtgacggctggcctccgcctgggtcatgggcctgtggggactgaacctggaagatcatggacctggtgcccctgctcactgggcccgagcctgaactgagccccagcgggctggcgggcctttttcctgcgtcccagcctgcacccctccggccccgtctctcttggacccacctgtggggcctggggagcccactgaggggccagggaggaaggaggccacggagcgggaggcagcgccccaccacgtcgggcttccctggcctcccgccactcgccttcttagagttttattcctttccttttttgagattttttttccgtgtgtttattttttattatttttcaagataaggagaaagaaagtacccagcaaatgggcattttacaagaagtacgaatcttatttttcctgtcctgcccgtgagggtgggggggaccgggcccctctctagggacccctcgccccagcctcattccccattctgtgtcccatgtcccgtgtctcctcggtcgccccgtgtttgcgcttgaccatgttgcactgtttgcatgcgcccgaggcagacgtctgtcaggggcttggatttcgtgtgccgctgccacccgcccacccgccttgtgagatggaattgtaataaaccacgccatgaggacaccgccgcccgcctcggcgcttcctccaccgagaaaaaaaaaaaaaaa

SEQ ID NO: 31 - DNA, 2008 bp, Homo sapiens
atgcgcgaaagatccgggaagtgtgatcggaaaggggcggagcatcagggagagtctcgcggacataggcggttcggcgcggaaagcgggaggtggaggggcggcttggggcaagcgcgcgcgcgcagtgcagaagccagccccccgcggctgaggtactcaaggtgcccaaaggcggggtagtgacctcgcgcgtgcgctgtgcccgcggcagcgccgggtcctagtgtgtgggttgttgttggcaccgcacggcgcgtgcgcagtgaggacggcggagggatttgcggccgggacccaccccctgctccagtcgctatcggaggccgcgcgggtggctgagcagcggcctggtgcgctcgcttagcgggcgacggaatcagacggacgtggacgcccccggagtggaagccgaagcaggagttgttgttgctgaggggctgccgcagccgccgcgagcctccggacagacgccagagcgaggagggcgctacgcgacttggcaagatgacccagttcctgccgcccaaccttctggccctctttgccccccgtgaccctattccatacctgccacccctggagaaactgccacatgaaaaacaccacaatcaaccttattgtggcattgcgccgtacattcgagagtttgaggaccctcgagatgcccctcctccaactcgtgctgaaacccgagaggagcgcatggagaggaaaagacgggaaaagattgagcggcgacagcaagaagtggagacagagcttaaaatgtgggaccctcacaatgatcccaatgctcagggggatgccttcaagactctcttcgtggcgagagtgaattatgacacaacagaatccaagctccggagagagtttgaggtgtacggacctatcaaaagaatacacatggtctacagtaagcggtcaggaaagccccgtggctatgccttcatcgagtacgaacacgagcgagacatgcactccgcttacaaacacgcagatggcaagaagattgatggcaggagggtccttgtggacgtggagaggggccgaaccgtgaagggctggaggccccggcggctaggaggaggcctcggtggtaccagaagaggaggggctgatgtgaacatccggcattcaggccgcgatgacacctcccgctacgatgagaggcccggcccctccccgcttccgcacagggaccgggaccgggaccgtgagcgggagcgcagagagcggagccgggagcgagacaaggagcgagaacggcgacgctcccgctcccgggaccggcggaggcgctcacggagtcgcgacaaggaggagcggaggcgctccagggagcggagcaaggacaaggaccgggaccggaagcggcgaagcagccggagtcgggagcgggcccggcgggagcgggagcgcaaggaggagctgcgtggcggcggtggcgacatggcggagccctccgaggcgggtgacgcgccccctgatgatgggcctccaggggagctcgggcctgacggccctgacggtccagaggaaaagggccgggatcgtgaccgggagcgacggcggagccaccggagcgagcgcgagcggcgccgggaccgggatcgtgaccgtgaccgtgaccgcgagcacaaacggggggagcggggcagtgagcggggcagggatgaggcccgaggtgggggcggtggccaggacaacgggctggagggtctgggcaacgacagccgagacatgtacatggagtctgagggcggcgacggctacctggctccggagaatgggtatttgatggaggctgcgccggagtgaagaggtcgtcctctccatctgctgtgtttggacgcgttcctgcccagccccttgctgtcatcccctcccccaaccttggccacttgagtttgtcctccaagggtaggtgtctcatttgttctggccccttggatttaaaaataaaattaatttcctgttgatagtgggcaaaaaaaaaaaaaaaaaa

SEQ ID NO: 32 - DNA, 1395 bp, Homo sapiens
atggacttccagcatcgccccgggggcaagaccgggagcgggggcgtggcctcctcctccgagagcaaccgtgaccgcagggagccgctccggcagctggccctggagaccatcgacatcaacaaggacccgtacttcatgaagaaccacctgggctcctatgaatgcaaactctgcctgacacttcacaacaatgaggggagctacctggcacatacgcaggggaagaagcaccagaccaacctggcccggcgagcagccaaggaggccaaggaggcccctgcccagcccgcgcctgagaaggtcaaggtggaggtgaagaagtttgtgaagatcggccgcccgggctacaaagtgaccaagcagagagactcggagatgggccagcagagcctcctcttccagattgactaccctgagatcgccgagggcatcatgccacgtcaccgcttcatgtctgcgtacgagcagaggatcgagcctccggaccggcgctggcagtacctgctcatggccgccgaaccctacgagaccattgccttcaaggtgccgagcagagagatcgacaaggcggagggcaagttctggacacactggaaccgggagaccaagcagttcttcctccagttccactttaagatggagaagcccccggctccacccagcctccctgctggcccccctggggtgaagcggcctccacccccgctgatgaacggtctgccccctcggccaccgctgcctgagtctttgccaccgcccccgccaggaggcctgcctctgccacccatgccccccacagggcctgcgccctcagggcccccgggaccaccccagctacccccgccagctccaggggtccaccccccggccccagtggtgcatccccctgcatctggggtccatcccccagctcctggcgtccaccccccagctcctggcgtccatcccccagcccctggggtccacccaccaacctctggggtccaccccccagctcctggagtccaccctccagcccccggggttcacccaccagcccccggagtccacccaccagcccctggggttcacccaccagccccaggggtccatcctcccccatcagcgggggttcacccccaggccccgggggtgcacccagcagcccccgccgttcaccctcaggccccaggggtgcacccaccagccccagggatgcaccctcaggccccgggggtccacccccaacctcccggggtccatccgtcggctcctggggtccaccctcagcctccgggagttcacccctcaaatcctggggtgcaccccccaactcccatgcccccaatgctgaggcccccacttccctccgaaggcccagggaacatacctccccctcccccaaccaactga

SEQ ID NO: 33 - DNA, 3513 bp, Homo sapiens
  modified_base (3414)..(3414): a, c, t, g, unknown or other
  modified_base (3435)..(3435): a, c, t, g, unknown or other
  modified_base (3469)..(3469): a, c, t, g, unknown or other
  modified_base (3501)..(3501): a, c, t, g, unknown or other
cggacgaggcacgagtgaggggggaggcggtggcggcggccattttgagccgctgccgccattggagtgggccccccccctttcccccttcgcctcctgacaggaaaggtttaagggggacagagccctgggaggccgggccgggctcgggggccaccccgggggcccgggccatggatgtgcgccgtctgaaggtgaacgaacttcgcgaggagctgcagcgccgcggcctggacactcgaggcctcaagaccgagcttgctgagcggctgcaggcggcgttggaggccgaggagcctgacgacgagcgggagctcgacgccgacgacgaaccggggcgacccgggcacatcaacgaggaggtcgagaccgaggggggctccgagctggaggggaccgcgcagccaccgccgcccgggctgcagccgcacgcggagcccggcggctactcggggccggacggacattatgccatggacaatattaccaggcagaaccaattctacgatacccaagtcatcaaacaagaaaacgagtcaggctacgagaggagaccactggaaatggagcagcagcaggcctatcgtccagaaatgaagacagagatgaagcaaggagcacccaccagcttcctcccgcctgaagcttctcaactcaagccagacaggcagcaattccagagtcgaaagaggccttatgaagaaaaccggggacgggggtactttgagcaccgagaggataggaggggccgctctcctcagcctcctgctgaagaggatgaagatgactttgatgatacccttgttgctattgacacctataactgcgacctccacttcaaggtggcccgagatcggagtagtggctatccgctcacaattgagggctttgcatacctgtggtcaggagcccgtgccagctatggggtcagaaggggccgtgtatgcttcgagatgaagatcaatgaggaaatctccgtgaagcaccttccgtctacagagcctgacccccacgtggtccgtatcggctggtccctggactcctgcagcacccagctaggcgaagagcctttctcctatggctatggaggcactgggaagaagtccaccaatagccggtttgaaaactacggagacaagtttgcagagaacgatgtgattggctgctttgcggattttgaatgtggaaatgacgtggaactgtcttttaccaagaatggaaagtggatgggcattgctttccgaatccagaaggaagccttggggggtcaggccctctatcctcatgtcctggtgaagaattgcgcagtggagttcaacttcggacagagagcagagccctactgttctgtcctcccggggtttaccttcatccagcaccttccccttagtgagcgtatccggggcaccgttggaccaaagagcaaggcagaatgtgagattctgatgatggtgggcctgcctgctgctggcaagaccacatgggccatcaaacatgcagcctccaacccttccaagaagtacaacatcctgggtaccaatgccatcatggataagatgcgggtgatgggcctacgccggcagcggaactatgctggccgctgggatgtcctgatccagcaggccacccagtgcctcaaccgcctcatccagattgctgcccgcaagaaacgcaactatatcctagatcagacaaatgtttatgggtcagcccagagacgaaaaatgagaccatttgaaggcttccagcgcaaagctattgtaatttgtcccactgacgaggacctaaaagaccgaacaataaagcgaaccgacgaggaagggaaggatgtcccagatcatgcggtcttagaaatgaaagccaacttcacgttgccagatgttggggacttcctggatgaggttctgttcattgagctgcagcgggaggaagcggacaagctagtgaggcagtacaacgaggaaggccgcaaggctgggccaccccctgaaaagcgctttgacaaccgaggtggtggtggcttccgggcccgcgggggtggtggtgccttccagcgctatgaaaaccgaggaccccctggaggcaaccgtggcggcttccagaaccgagggggaggcagcggtggaggaggcaactaccgaggaggtttcaaccgcaacggaggtggtggctatagccagaaccgctggggtaacaacaaccgggataacaacaactccaacaacagaggcagctacaaccgggctccccagcaacagccgccaccacagcagcctccgccaccacagccaccaccccagcagccaccgccaccacccagctacagccctgctcggaaccccccaggggccagcacctacaataagaacagcaacatccctggctcaagcgccaataccagcacccccaccgtcagcagctacagccctccacagccgagttacagccagccaccctacaaccagggaggttacagccagggttacacaggcccaccgcctccacctccaccaccacctgcctacaactatgggagctacggcggttacaacccggccccctataccccaccgccaccccccaccgcacagacctaccctcagcccagctataaccagtatcagcagtatgcccagcagtggaaccagtactatcagaaccagggccagtggccgccatactacgggaactacgactacgggagctactccgggaacacacagggtggcacaagtacacagtagccagtgtgacccagaggctcccggaggcccctgccggcttcctccaccagcgcctgcctcggcccctcctctgcccccgccagatcccgtggtgctggggatggggtcatcccagggctgcctccctccagcccactgcctcccctctgagggggttccttcccctccatagggccaggcatttttttctggattcaaacaggcaacaatgaccttttattttctgtttgtccccacctccccaggcttccacctcctgttcttcctaccttcttcctttttgactaaataatccccacctcccttgatcatacagtgaggctacagtgactgaggggagaatcccctcctgttcactctcccaaccctgctccagcccctcagcttcccagaccctcatgcagttggttgtaaattctcccaggagctgttttactgtctacttttcagggttaaaaaaaaaatcaaaacttaaaaaaaaaaaagtttaaaaagcaaaatggggagggggaggaagcagtgacttttttttggtaattatgcgcttttttttaatttttagaatttgtctttttactgtggggtggggctgttgatatttcatcaagataaggcatttctttcccggagttcaggtgactgagggaagggcccccaaaacaaaacacanccaaaacccaaacccccaggnttcctctttttaaccccaacttttttttaccggntggcccccagtttcccccttaacttttggcancacaagggtttt

SEQ ID NO: 34 - DNA, 2519 bp, Homo sapiens
aatcttggtcgctaggacacggctaacttccgctttcttccccctctcctaggctcaaactagtcaaatcttgttcactcgaccaatggcaaatcggaagtgggcgggacttcacaagtccggaccaaagaaacgcgagcttagccctgggtagcgcggccaatggccgtggagcagcccctgtaaactggctcgggcgcccccacgcccgcccttccttcttctcccagcattgccccccccacgtttcagcacagcgctggccgcagtctgacaggaaagggacggagccaagatggcggcggccgacggcgacgactcgctgtaccccatcgcggtgctcatagacgaactccgcaatgaggacgttcagcttcgcctcaacagcatcaagaagctgtccaccatcgccttggcccttggggttgaaaggacccgaagtgagcttctgcctttccttacagataccatctatgatgaagatgaggtcctcctggccctggcagaacagctgggaaccttcactaccctggtgggaggcccagagtacgtgcactgcctgctgccaccgctggagtcgctggccacagtggaggagacagtggtgcgggacaaggcagtggagtccttacgggccatctcacacgagcactcgccctctgacctggaggcgcactttgtgccgctagtgaagcggctggcgggcggcgactggttcacctcccgcacctcggcctgcggcctcttctccgtctgctacccccgagtgtccagtgctgtgaaggcggaacttcgacagtacttccggaacctgtgctcagatgacacccccatggtgcggcgggccgcagcctccaagctgggggagtttgccaaggtgctggagctggacaacgtcaagagtgagatcatccccatgttctccaacctggcctctgacgagcaggactcggtgcggctgctggcggtggaggcgtgcgtgaacatcgcccagcttctgccccaggaggatctggaggccctggtgatgcccactctgcgccaggccgctgaagacaagtcctggcgcgtccgctacatggtggctgacaagttcacagagctccagaaagcagtggggcctgagatcaccaagacagacctggtccctgccttccagaacctgatgaaagactgtgaggccgaggtgagggccgcagcctcccacaaggtcaaagagttctgtgaaaacctctcagctgactgtcgggagaatgtgatcatgtcccagatcttgccctgcatcaaggagctggtgtccgatgccaaccaacatgtcaagtctgccctggcctcagtcatcatgggtctctctcccatcttgggcaaagacaacaccatcgagcacctcttgcccctcttcctggctcagctgaaggatgagtgccctgaggtacggctgaacatcatctctaacctggactgtgtgaacgaggtgattggcatccggcagctgtcccagtccctgctccctgccattgtggagctggctgaggacgccaagtggcgggtgcggctggccatcattgagtacatgcccctcctggctggacagctgggagtggagttctttgatgagaaacttaactccttgtgcatggcctggcttgtggatcatgtatatgccatccgcgaggcagccaccagcaacctgaagaagctagtggaaaagtttgggaaggagtgggcccatgccacaatcatccccaaggtcttggccatgtccggagaccccaactacctgcaccgcatgactacgctcttctgcatcaatgtgctgtctgaggtctgtgggcaggacatcaccaccaagcacatgctacccacggttctgcgcatggctggggacccggttgccaatgtccgcttcaatgtggccaagtctctgcagaagatagggcccatcctggacaacagcaccttgcagagtgaagtcaagcccatcctagagaagctgacccaggaccaggatgtggacgtcaaatactttgcccaggaggctctgactgttctgtctctcgcctgatgctggaagaggagcaaacactggcctctggtgtccaccctccaacccccacaagtccctctttggggagacactggggggcctttggctgtcactccctgtgcatggtctgaccccaggccccttcccccagcacggttcctcctctccccagcctgggaagatgtctcactgtccacctcccaacgggctaggggagcacggggttggacaggacagtgaccttgggaggaaggggctactccgcccacgtcagggagagatgtgagcatcccgggtcactggatcctgctgctgtaatgggaacccctcccccatttacttctccacctcccgtcctccccatcattggtttttttttgtgtgtcaactgtgccgtttttattttattccttttattttcccccttttcacagagaaataaaggtctagaagtagttggtcaaaaaaaaaa

SEQ ID NO: 35 - DNA, 32767 bp, Homo sapiens
gagacgatgtcttgctctgtcacccaggctggagtgcagtggcatgatctcagctcactgcaagctccgtctcccgggttcacgccattctcctgcctcagcctcccaagtagctgggactacaggcgcccgccaccacggctggctaattttttctattttttagtagagacagggtgtcaccgtgttagccaggatggtctcaatctcctgaccttgtgatccgctcgcctcggcctctcaaagtgctgggattacaggcgtgagccactgcacccggcccagatatattaaattagaatatctagaggtggagcctgagtatctgtatttttcagagtttcaaatgatcgttcttcaaatgattacactgtgaagtcagatttagaaatgactgtacccaaggttggctaaaagatacacaccctggttgattctacctgaagagagcaaataagatacacagcaaagttgtagatgttttccctgccagtagaatacttgcgggttaggccatttaaaaccctgccagagagttttgaaacactgtggagggctcccaaatcaacttgctcaatggttctccatcccttcaggctacttgggcttaaagccaactgcaagcttagagcctcagagtgacctaggaatggggtgaccatatattctaggttgtctcatacagactagccagcactactcagccgcaagtaatagcatccaggcatgctcagaagtgtccctttggaggaaaaaaacaatattgtcacaaatgaattggcaatggcctgtctctgattcttatacctggaatatactggaagtccctactcatgctattttctagcagaataggcaaaatttctacattccaggcatgtcaggcctttccctgattcctttctctaatgtcactcgtctgctgtcttttatcacagccattaaactgcaccctaacttaaagaggatcccttatgttccaatctactcatccctcagatctttctttctctgaaacacagggttaatgagactgacatccttccatcacatattttctcagctactcagtaaaagatgtaaatgtttaaaatagtttaaactatttttcagttagtccaggaaacataaaatggcatgcttgcacataaaccattgtttagggtgggggaagtgtttttaattttgccttaaaggaaatctgcatgatccacaggctatgcaactaccaagggaattagttggtagaacagaattacacctgcacagaatacaaatttcctgcctttcatgggaactatgttgatgtttcagatatgaaatacatcttgttttctttattgaacctcgagaagatgtctcttgttggtcattatttcatggcaggggaagtacatattcctaaagacacaaccgagtttccctttaaccatcattagttgggctggccattaagaaccagacgcttttattttcaaagagacttaagttttgatgttgtacatatgtgcctaatattctatctcatagcaatttaaaggtgacgttttaaaaagctgcattcagtgtataaacttctcctgatcccagcaaggatgttgtgatgattttatttaaaaaggtaagttgtgtctagatatggcagtgggtcatctcatgcatggtgcagatgtcaaacacaattacattttcttatttgcaatgactaraaaaagaagctgagcccaagcagtgagaagtaggagattgggaggcaagaagcaaaggaaaaaagtacatgagcaccgttctccctgtcctgccacttgctccattatggactgggctgcgatatctcatatcccagctccacaactcccaacaaccatttatgtgcatggtgcttccatgtgtgatgacccaatcaggctcaggtgtggactgagtagttaaattataacccttgtctctgaagagtttagggcttagtggggaaacagacatgtaaacaaacctgagtgaggtcatgtaatcaaaggacaggccacagtcaaccacaaagaagagagttctcagcagtctccaaagccgaacatatgtttaccaggaacagggtcccagcagagggagcaacaggagcaaccagagccttgaggggtcgtggcctgttctgggcaccagcagtggatcaatgtggccagagccagggatactagcagaagccagagcagcagggcctgccttgtccagcaaaggcatttgtctctttgtaggccacagcgacccacagagggctttttaggccagaaaaaagccattaaggccgggcgcggtggctcacgcctgtaatcccagcactttgggaggccgaggcgggtggatcacgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccaggaagcggagcttgcagtgagccgagattgcgccattgcagtccgcagtccggcctgggcaacagagcgagactccgtctcaaaaaaaaaaaaaaaaaaaaaaagccattaaaaagggagtcatgtctcttgttggtcattatttcatggcaggggaactacatattcttaaagacacaaccatttcctcttaatcctcattagctgtgctggcaattaaaaaaccaaaagtttttactttcaagaagatttaaataacttctgagggtgtacatatgtgcttaatattctgtctcacagtaatttaaaagtgaagttttgaaaagctgcatcctgcgcttgtcagaaccatgtctgatgagatatcccctttaaagggctctcggtgcaatggggcaaatcaagggggtttgtgcaagtgggagtgagacaggagatggggtgcttcttccagcactccctataggctgactgagtgacaaagatcattttactgacacctccaatggccctatgagatgggtactattattattatcaccatcatattccttttgcagataaggaaactcaggcttagcagattgccagaacaacacaggcaggaagtggtagagtcagggtttgaacccaggtagtgaaactccaaagcccggattcttaaccactgtcctccagtgcctctctgtaataagtcatgatcccagaagccattggtgtggccacaatatggaaagagatgacagtgtcctcacactgggtgagcagcttatggtgattccagacatgatctctgttgggagtgacaggtctgagcttctaggatcagaccctagatcttggcaagtggtttgaggaaagagaaggaccaatgtaaaaccccaggcttcaaggaatgtggatgctgggcagggaggattaagccccaaagaccagaaatggggtacacagggcaggtgtggccagagtagaactagagtagaacttccagtgactagaaatagaaccagacacgttgcagtggtggataaggtagaatcgcttaagtctttaaagtgcccctgatcacccaagttggccagagaccctggggtggggctgattctgtctggatatacggggaggggtaagcatgaggaaaggaagcaggtcctgacaggtactttgcactaaacagctccttataaggttctcaatttgcctgctcaatttctacagacatttgtgggaccacaccagtacattgtaaaagcaggaaacaattgagaaaaacctgagttttatgttggtaggagaaatgcctatggaatatggcaaatcgtttctctgagacttcctccctagtaattacatatttgttctcaaaaacaaatgccagaaggaagaagcagatttaatagtgcattttacaaggcaccattaatctctaagaagaacaattaaaatgtctcagcaatcatggttcactgtatatcttttctatcttcttagaagtaatatatggctggaaatgggcataccaaaatatgtcaaggaagtggaattgcgttcattagatttcaccactaattattttagttagcttcacagatctctcttccttgcttgttcttgagagcgaggctttttagtaggaagagaaattgtctaaaacgattaataaccacaaattcaccaaactattttgggtaagtccctctatttctctaggtctaaagctaggaataagagtcattctcatataatgtactgtcccagaaagggcattatattagtctgttttcacgctgctgataaagacatatccgggattgggtgatgtatttaaaaaaagaggtttaatggactcacagttccacatgcctggggaggcttcacaatcatggaggaaggtgaaaggcacatcttacatggtggcagacaagacagaattgagagccaatcaaaaggggaaacccttataaaagcatcagatttcgtgggacttatcactaccacaagaacagtatgggggaacgccaccatgattcaattatctcccacaaaatgggaaaattatgggaactacaattcaagatgagatttgggtggggacacagccaaaccatatcaggcattcaaccaatatttgggaagcaccagccctgcaccaggcacggagcacgtcatgagtcctgccgtaccacagcctgcctgacagacctcagtcatcctctggagcttgcctctgacatctggacctcctcagaatcagcatctcttctccttgcccccgccatcctttgtttttatctctgctgtggcattcatcaaagccttccaactatcctgcgtcactgtccttcagtgtcctctctcctctcccttccttctcaccccactttgtgcctgtatccttcaagcagagcaatggcaccctcacttctgtggctgcccagtgccccatgcagagtcagacatcgaaaatagatgctgaattcagttgacactctgaaattctttttaaagtaagttaatgtgtgctttgaatgaaaagacactgggattacattattgagtgtctttcttcctttgccacttttgtccctattggccatatttgaaaatcttgttggaaaaaaaaattcaagaacttaataaataaattcaaaaacatttagtctatttacttaggtgaagagaaaactcattctaatatgtgtgtatatttaaaatatttgttatttagactttttttttaagtctccaggttgaggaggacacaaatatatcctcctaaaccttccagtaagcaagctgtggcatccagatgatctcctgggtcatgggggataaggctaatctcctaggtgtctggcagacaggacaggcaaattcccagaatgccaaaatataccatctgctgctgtttggcattgcccttaagtccagagtgtggaggctgggggtgggtctctggctacaggagaagtcccctggcaagggaggggtgaaaggagtgcctgttgaaccccccatctatccccgcactatggcaagattgagaggaatgactagatcagggaatggcccgaaagaaaaatccaaaacctcccaaccctggacaaggccacagctttgagaaaccgaagcctctgcttccttctctttggctttactgcttctagatgcaaatacacagagctctgagattttgtgtgctgggaggtgataactgttaaccctctattccaatagcacagaaatttctctttgcctcagaagtggtttctcatagatctcagatctcttttcaggaaaaagaaaaacaacaacaataacaacacattaatgactctgaaagagtcagacaccattaattccattattggtgtctgtgccaagtgaaatgaacgtcagctcttttcccagatatgtttccttcttttgcctcctataataagagatgattttacygtaataatataagactcatcaatttgactccaaatagctttcctatcaacaggctaagtgtaaaataccaggatcattattcagttgagaatagatagaactaggaagtagccatcaaaaaagaatgatgaggtgcattgtggatttggggtgtaacttggtatctaacatacagccagaatcacagtcatagcacacttaatattttatcagaaacttgcgtgaacaagttaagaggactctcaacttaaaaatgacaccaattgcaatgatcttgttaacatttgtgatgaaaataatagcaaagtgacttagacaaattacaatagcccataaaaataagataaagtttaacacaaagtaagatgatgttaaaagacttgaaataaaacagatatgttaagtaggcaacacataggtaagcatataaaaacaagaagataccaggatagagctgtcatttttgtgggagcctgtgatgtggaaaaccaagatgcctggtgagtataatggatatggaaaccccccttgtaataattccacagttccaaggggccaaggtctccaggttgagtcactattgtaaacacacccatagatgaatccacatgccatacctccttgagtaagtggggactcaaactaggtctgtcaattgttccagaaaattaagcatctaaataatttaatgataatttaaaagaagcacaatgaaatatttcaaggaatgtcacatacaagattctgtacctcttctgctttggttagactcattcagaataggttcctgctttgatcttaagagggaggtagagattctggagaagccctagggaagagcaaaaggaaaggaataaggagccaagaggaaacccagggtaaggctgaggagggactgtttcgtgtaggtgatttattggaagggttggaaggaaacatggaatgacaattacctttggttattgtcaggttagtatgagacttacaagaaaagcactgctcagacgcaattaccattcaagataagaaataataggaaaggctagcacacttagctttttatttaaaaaagtgttaggtaggctgagcacggtggctcactcctgtaatcccagcactttgggaggccaaggtggatagatgacttgagcccagaagcttgagaccagcctggacaacatggtgaaacctcatgtctacaaaaaaatacaaaaattagccaggcatgatggcatgcacctgtagtctcagctacttggggggccaagaggtgggaagattgcttgagcccaggaagtcgaggctgcagtgagccatgattgtgccactgcatgacagcctgggcaaccgagtgagagcctgcctcaaaaaaaaaaaaaaaaaaagtgttaggtgacatgagagaagatcttccaagtaataagagtggctaatcccaggaatgtgtcaccagaggttattttgtaatagtcgtgtgttaaattccttatttgtctatataacttctcaaatccttctgcctctacagttatagtttaactggcgcataacagccttcacacacagcctcataattaaacatagacatacatatgaacactttcccctatgccagcaggatacttggtttgtttaggggcaaagaggaattgatgtggcgttgtttcaatcagtggttgaaaatgcaagtggtaaacattgaaaaatagaacactgcaaaaggcatgcattgtatataccaaaaggtcagcatgaagcattatctgtatggcaagcctgcccatccactccctcctacacgttgcatattcacacagttttgcagcttgtataaacccctattgtgatagaaactcatgaaagagtgtggtctctgcgaaagctggctgttctgtgaatttagaccagtggttcttcaccctggctgcaaatcatctggggaacatttaaaaacactgttttaaacaccccaaccctagaaattctgatttaattggtctgtggtggggcccagaactctgtattctttttttaaggctctcaggtgctgctaatgtatagctaaaattgggtctggtttagactctcagaatttcttaataattaaacactttatcatgacaagactttcaggaccttaaaggccacagtggggtagttatcatttcactaggtcctcatctggggaggtccttggcatttttactggaatatatttgtcactcaaatttctattacaaaaaattctttcttgcacactgctttagcaactacatgagatatactttgtacatagcacaaatctcatatcacttatgtaatccagctctgtggttccttcctttcctttgcctgtttatttttaattcttcccaagaggaagcttagccagttagaacaccagagtatcatccccctcccccttttcccacctgagttcatggcttagacatactaggaatgaagctgacaacatgcactagtttttttcgaaattatgcagcaaaattcccaaagtgcgagtggccacagagatcttcacagggcccagggacaggcagacatcattctttctccagttcctggcacagaaaagagaccttaggttactgagaagataccagtccctcctcagagcagacaaggaaactgagcctcagaatgaaagactgaatttcagtcctttcttgaacatggacctccagggttatattgggccttggaaaaggcacttacactctggactgtagtttcttcatctataaaatcaagaggcagaaacagacaatctctaagttgcctttatttataaaattccgagattctagttgaccagtattcatacaagagttgaagcctgtaagagtgcagaaagcccacacaaagagacagtggaagacctctcatcagtagtatttttattaccctcttcctaggttttaccagtcaacatcctcactgttaatatacagaccgtggtatttaattaaatcatctttgaaatactgagctatcaacagatggcatgctgaatgcaaaaggaccacaaataaatatttggtactgaagaagatcaagagttggagttcatttcccattctgatctgggctcagaactctgtggtcttccctctaatcatccttgccaccaaattggctgtatctgttctaagatggatcagaaaatcagttccaaagttggctacaaactttcaggtttgggttttgttttgtttttttgttttgttttgttttgtttttgcaaccagccaattcatcttagttcacatgacagagaagtgcataattacttgcaactttagttagagcagtggccttaagaaggtctagctaaataaaaagtgctcagactttctgagtgctgacagttgtcaaattcacctagttcacatggccccatttctatcgtttgttttgttttgtttttgttttttaacagcccatctgtgagcaataggatcagatgactaagagctacagggcagaaacactgttacttagagtcaaattttcccattacctagctgtaaagagtttgtttctctctgactcatataaagtttaccatttaggcccctgcatgattttaattccatcacttaacaccccagccatatgattctgaaggtaaacatgaaggcgtttgaattccagaccacctaaacattcttaaggaaatcatcatctccacgggcagagctatgccaaaatctgtaggttttaactcaaatttcatgataagcaaaaattgaattaatttgtcttccattttgttcacctttttgccaaaattatgcctggattagaataaataaattcaatcaatgaatgcaatcactaattcttacgccagataataacacattcagaattctcctttccctgggagattttatcaggttagtgttcttgtaaacaggagaaagagaaaaatataacttagtaaatagcagtattcactaattcattcatttattcaacaaatattaatttactacctactacattccagggagcttagagtctagtatcagaaataataaccacacacacacatacacacacactacattaaataaggatgtgataggctagatgaaataaataaataaataaaaggtccaggtgagaaaagaaggtgggggctagaaagaagtcattgaagaaaaaacatttaggttaaaacattatgaataacttagagtgagccaagtgcagagtgctgaaggagtgctccaggcaaaatcaacagcaaatggggagtccttgatgtagaaaagggtttgaggaattgtcctgggagaaatactcaagattccagtctgaattctagaggttagtgatttagagaggcaagtacgaaaatgacgtcctctcttaccttaaaagtaagtgcaccatagaaggaaatcacccttccttggtaataattcctgagtgagcctgagaagccagaggccatctctattttataggcactgtccccttttcagttacccatggctagctcattgaccttgtcctggtcgtttcctcatttcacttactccatcctcaaaacgtagacgcttcataaatattgtataaatgaatgaactcacaaagtcacagtacagcaaggcaaaagtgcctgcaataaacaagcattctaggctagaaatatttctcaacttcaaattgtgtcttattacattgtattccgattttctagagtggtagttctcagtcaagggaaagtttttcttcccttccaggggatatttggcattgtctggagatagttttagttgtcacgatttgggggatgcttctggctcaacttgggtagagaagcggggatgcttataatcatcctacagtgcacaggacagtacccccacccacactccagtaatgaagaatcattagacctaaaatgttaatggtgtccaggtagaaaaaccctgttgtagaggttggggactgcgtcttgacagccacattatacagtgtatcaaacaattctgtataatgggctgtaattatccttgcctagattttgcaagaaccctagtgtgtatctttttcctcacttgccaagcaatgttcaaacctgcagagatttatttcattcattttctgtgtgtttagtaaacagactagaagcactggaggaaaaaatattccagcaatgaggtaagacgaaagctattagtaaccctagtttaacttagctgaatagtaggaaacaacctctaccgtgaggaagtgtattgtagaaactgaaaagacgctaatgatgtttaaaaagctgtagttcaaacaaatgtgcatgcagaccaatgggtagactgaaaatgatgaagacatttccgtttcttgtgtctttgatagaaaagaaagagcttttattttctttagtgtggcaatcattcagatttgtcccatgacatgcccagaaggttgaagaataacaaactcccaagtgtaaacacagaatttagcgaagaatccaggcctctggatgaatccctgtaattgcatgtttggataaaataagattttcatacattaaacaaggtaggatttttctatctgggacggaactttcaacacttggaggggttgtagttatttctcctcaaagatggcaaacatgagtgccccgagttatccctcctctctgttcaagttcgctaactaatcacccagtatccatgctatcgctggcccttctgtggcctatttttatactgttcactgttcagtgtcacttgtttggtaacactcaacatcaacatgtgctaccaaattgacaccagaggacaaaaaagaatcaagatatgtacagcctgctttgtactgagccagctgccactagatgttttttgtgataatgaacacgtgaggccatgtggacgcgagagatggctccgggttccctcagacggctcacagccagctggtctgcagtgcggttttagattccgatgtgggaaccccataaaaaagaatatgcaggccaggcgtggtggctcatgcctgtaatcccagcaatttgggagcctgaggcgggtggatcacctgaggtcaggagttcgagaccagcctcgccaacatggtgaaatcctgcctctactaaaaataaaaaaaaaaaaaattagtcaggtgtggtggcggatgcctgtaatcccagctacttgggaggctgaggcaggagaatcgcttgaacctgggaggcagaggttgcagtgagcaaagatcgcaccattgcacttcagactgggcaacaagaatgagactctgtcacaaaaaaaaaaaaaaaaagtctgcaggctgcataaagaggtatgaaaatgttccagaaatcccaaatcctatccctgaggttcattttggtgagggaatgtgtgtgcattttctagggcttccctaaaaaagtatcacaagctggatggcctaaagctacagaaatttcttggggacaaatttcatgattctggaagctagaggtccaaaatcaaggtgtcagcaaggctatgctttttctgaagcctatagggaaggccttccttgtctctcctagtttctggtggtttgctggcaatgtttggcattctgtggattgcagctacataactccactctgcctccatcattaatggccttctgcctgagtgttttcatatgaccatcttcatataaggacaccagtcatatttgatgaggttccaccctactccagtatgacctcatcttcactaactacatctgcaatgaccctatatccaaataaagtcacattctgagtgtctggggattagaacttcaacagagcttgttgaagggggcacaattcaatgcataacaggatggaaactagaaacgggtatgtttttatcagtgtagaaagatttagcttaatttttcaaagtgtaataaaaaccccaggaaaactcatactccctcctaagaagagcaaaagatggagaaacccgatggttaccttcaaacaaaaggaaaggaggaataagatgaaaaggaattaatccaaagcaaagagagtggcttatatggaatgttggtgcaactttctctgacacatctgtgcactcatcagctggggcatcatctccctggggtacatttggtcactgtgtgcctcatggtaataaactccagaagcctcattgacttgctagagatgagctcatccttcttgcttgcttaatggcaaaatacaaaataagcagtcactgacatggaacgatttcaggaatgccaaaaggttctccttttccaaaatatctcttccatcttcccaatactgttactgacatcactaacacctctccacttccggttgagacacctgggccagagctcctgatgtggcaggcagtgccctaaacgttttgcataaattaactgatgcccagagcaacaaccctaagatataggtactatcataccgcatcttacagataagaaacttaggcacaaagaggtttagtagtttagatgagataaccctgatgagcagagattcgaacccagcctccatgctattaaccaggacatcatattgcctttcatacatgctcttcaaaggcaacacagtaatcgattatcacactcactcacatctgattgtcacatttttcagatctgctctcctagcagagaatgaagcctaaggtatccttgtttctcaaagtgtcctccccagaccagctgcatcaaaatgaggggatgaggtgcaaatgcctggaccctgcccttggagcactgattcataatctcaagtcccaagaatctgcattttaacaagcatccccagaaatttcttaagtatactaatgtatgggaaccactgacactaaagaaatggaataaggggaacgtacaatgttacagtaaaccaggaaaagccagaaagacatgacaacacagtgaggactctggtagccaatggtcagtcaaatgcccaggggccctggccagaagagagttaggttgctgaggagtaagagtgatgctgaatgtggaggcttgagagcagaaggaagccagccagctatatcctcttgcttggatcacacaccctttccttggtggaaatggttatttgcagagttagagaaggcatgttttacagtttggatggcaggtatggatgtagacaataaagagcaaccagagtccatgggttcagaaatccccatgtgtttctgtttgaatgagacgcttgcataaacagcacaaggagtttggggtggggttaaagagaatggtgtggtatagggagagctgaatgaggaactgagagagcaaaatcctgtgtttggttcaatcactgattacaacctccctgaggctcggtctcctaatctgtaaaatggggggaaataatacctgccttgcaggtcctcacacacagggcatgatgtgaatccactgaggcatatagcactgtgtaacatgagttattgctattccaaggcccgtaaaaggctcttgccttggaatatatctgccacaccaatgcctgcagtccattaatgacacataaaggacactggagataacgatgtcccttgttctatgcatccctcccacccatgccagaaaagaaaacacagtcacctgaagtcattctaaagagtatgcctgcctcttttcctgcacagacacatatacacagacacgcacatacacagaccatgcacatacacacacatgggaaaacatgaggaaaagtggagacaagaggcaccaaaggacaaagtcacttttgtcgcctgtcccttccccagcagggctgacctgtgggctgcttctcctgcctcctccctgaagccccctcctcatcatattccagtgcgtgtccaccactttggggccaggtctacacaactgcagtgattcaggtcacgggagaaaacccaaacaagcacaaaacatgcttcaacctatattttctaaattgtttttctttaaaggtgaagacttctgagcttgaattatccccttgtcagtgggctttccatgctgtccaagtgacctaagtgataatcaacctccatttcattttgagaatggttgtggtattttagagctatggtgaataagaaaatcatttaaaataaaatgatttttatttatttattgtttttatttattttatcttaaatgaattttaaatcatttaaaataaaataatgggataaaagaggatgctaaaaataataaatatatatgtatcaaagtgtgcttgtaataccaggcaaagaattaataagagataatattatggttggtgaaatgttatgtatggctacatcctttcaatgagcatttatagttcctttaaaatatgcctactgaagaaatatttacatgctaattaacatgtgcatagtaccactaggtattatagaggataccagatgtttgtagtagacacagaccttgccctaagtcctggtcttgatgtagtcactttttagtcactacaggtgactacatttagtcactacaagtgaccttccttcaatggggaaataaaggactttacaaaagacgtagaagacaattcttaatataaaagtgatttagatcttcacaagtttgtgaagagaagcagatgagtgaaatagaacactatcaatgtaaaatattattctgaggcctctgtaatgactgggaagcaacaagagggaggtcatttcagagagagaggctctaggttccaagctggatgctcaggtcagtgactgcaggtcccctccacacccatcaccccacaccctaaccctcttcagttgctcacaaaggtagataaatacccacatttttgccctcttccatcttgaaaccctggaaacccttgcttccgccaggggaggttacttagtatctgtcaccccaagggaaccaacgtcgaagcccaagaataagagtcaatactcctaccagaggtttacatttttcccaggggtctaggtggatattcctgggaacccccgtcaacacaggcatctacagtacaatccaggcctcctgttttcagcaggggctgcaagagcactgcagccttttccccagaggtgtcagtttggcccagtaaagattgcccctgagaaaacacatgggcaattagagcaaagttcctatgttctggtaacatttaattgtgctatttctcaacctcctctgcacccacacactcacacacaacatttattccactgacttcaaaggaagctcaacgtgttaaaaatatgtgtgggaacaaagaagggagtttgaaattggtctaaactctgtataactgggtttgacacgtacattaggattttacaagtatgtatttaatctttttttaaaaaaagcgtttacatagggttcagaataatgacaataaatcaacatttctattgtccatttgtgtgttttcatagtaaataatgctcatttatccttaaccagtaatacatacttatgggcttaaattagcaaaagcctctcaaaaagtagctccactcatttatccaccagtgtccagatgccatccagcacatgaggagctcccagaaaggagcagggaacaaactagggctgtcaggagtggaggagaaagaatggcatatgcaaaaaggagctgtaattaaatccaagggaacatggcacactctagtcttttgcacgagacaaagggcaatcctggtaaaaatacagatccccaggccccaccccaaagagtctgatctgattctgaaatggggccggagaatctgcattttaacaagcaccttcaccaggtgatccttttgctgagaacccctgagaaatgagaaccctgtgctagtgctgaatggagcattatattccagagttgaagtttggtgatcagttttccagatggagctggtccttggtgcatacctgggtataaatccaagccaattcaggtatatgagctgatatttcaaccgaaacactatctatagcctaaattttttctaatattctgtttggtatgaattctagaaagttgtaaatgctatatttccttctcatctatttctggactttgtcccaagaccaaatcccagggcatctgatagacattcattgcatacatttttctgtaaacatgaaaactgaattgtctaatagaaaagggcaaggaagtagaaaataagaaatcatcatcagaagtggtttgttttggaattatattgtccagctgcataacaaatcacccccaaaattgagtcgcttagaacaacaaacattgatcctccacagtttctgtgtgttaggaatcaaagtgatttaatttaatggttctgctcagggtctctcgggggctgcaatccaggtctcaggctgggatcctttcaaggctgagctggggaaagatccatgtctaagctcactcacatggccgatggcgggattcagttcctcttaggctgtcagactgagggcctccgtgtctcagtggttttagccagagccctctctcagttcctttccacatgggcctctccacagggcaactcacaacatggcagctggtttccagtagagcaagcgagtgagagaacaagaaaggcaagcaaggtgaatgtcccagtcttttgtaacctcatctcagaagtgttaacccatcacttttgccatgttttattatttagaagcaaatcactaagtccagcccacaattagagggatggcattacacaagggaatgaacaccagcagacagggtcattgaaagccatcttagatgctgtctatcgcatctaagtgtgatttttccagatgaaaagaatatattaatttgtttcagtcttagtcgatgtgccatcccatttgtgctttgctaaaacttgtatcaatgtaaagcaaacattttctgatacaatttaggtagtgtattgtggtaatagagaccagtagtgttgaaaagatatgttgaggtcagaaattaagctcatgtttctaaaagaggagatatgtacaactactatgcaagccaacaggaaagagtgttttaagaatgctttctgctacaggtaactaaaaacctaaacagctgtggctttaaaataaaggtatatctaagtcacataagcaaaagtctaggggtgggcagctgctggcattgcttcagtagcttgataatggcaaaagcagcatctcttctatttccttggccttctaatcatgcatgtcacctcacaatcacaacataggcaacacctcatattctaagcaagatgaaaagggcaaagagtcatgccatatgcctctgtctcttttcataaggaagacaaagcttccctggaagtcccctctagcagatttcacttagatctcattggccagaactgagtcacatgcctgccttaaaccaatcactcaccaagaagactaacattatcatggcaagtctaaaccaactgtgactcatctctgaaatcaaaggattattaccattacccgaatccatcaggatcctgttggcagagaagtgggactgtaaattttgagcaggcaacaaacaagtcttctgtaaacttcttatgtgttgttttttatgtgttctatatatccagtagaatcacaatttccaataacagtctaaaaagatattttccaatagaaacagaatgtgtaagatcattacttatgaaatcccaaatgtacttaaggtttccttcttgaaaattccttattcaaaataaaatgtccagattttgaaacccagaaaagattctatattttaaaaatcctgtgcacatgtaaactgtttttcaaatattgccttcagatacattgaacagaatgaaatcttctgagatttactacatcagccaagtattatcaaaacaaacaggacagattgcttttcttgacgtctgctgcttgatttgtgttaactcatgtttctgaaattgtagtatcataagccaatgctgcacaaaggtatttcatgtcatttataaaaatctagtaatgtaaactgttaactccttataaagcatctgttgacacacaaaaatatcactgaagtgcatttatgcctttcttctttaggtctgcataatacttccctccagaaggccaagttgttccataaattacagaacagaaagttggttgtgggaggaatagctcaacctcatctgaggcatcccactctaagaaactaatggcacctacacctcttgggcattgagtttttaagcccatttttaattcttgttctgctcatattctaagtgagcacataaagtgctgctccaagcaagaccagcccttgtagaagggcaagtgcagtcagtcccctaggaaacgggactggggagtgatcgtttcaatgagagataaatcaaactgatgctaaacatgaacaatgagcccattagagattgtgagaaagaggcatcatcatccactcaacaataggcctgtgggacctcttgatagcctgaggatgtttaatttcaggtgcaggtatccagaatgtagcagctagactgatcaaggatgtgtgatgacagcaagcagtagtggaagagcccaggagagttcctaagcctgaattgcaatcctgtgctgccataaaatgggaagatatacttggtccagtcatctgacagctttggtcatcaatttctctatctcatatgtgactctattgctttaagaatccctttagctttaaatatctatgaatctgctgaagcagctgtgctttgattgatgtggatctctgaactcccttaaatacaaagaccaattatttagccgagctttgttggattcagtgcattctgaatacatgtcaaaatatacttggatttgtaaaaaatattccttcctgtttttttcaccatagatagatgtacaaaaatgtccgtgttcacaccgtggaaaggacatttctcataaactcacacagagatacctttcaagtcaatgccttagaaagcaatgagagatttaaaggagacctagagatatgaatggagtaggcagagaaggtatgtgaggagaatgatgtaacttcctagggaaaaagtatgaagcacaaggctggacatagacctgggaatcaggaaattagagttctaattgcagcttttccattgattcacttgggatcttgagaatatctgtctcattttaatcattctgggccacagtttccatatctgtcaattagagtaagagtccctggctgggtgcccaggattgtgagaacataccattcagagccataaaaatgcaatcagtaccaataatgtactagtaccagtacctaggatgcaaaacatcctagatactaggtgtcctaacttaaagtggaaacattaacaagagtaattctttgaatcatcaaactgggaatattttaggaagcatatctatctgggtgaaaactaagcaaataagacaattgtaaaggcttgtgatctcaggaatacaaaggcaaaaatgcgcagacttgaaatatgacaagttctagttttgtcacttagcatctctgtgaccttggataatttcttaacccccggcagtattctcatctgtaaaatgggaataatgacatgcacttcagtggtttgtggtgaagattattacaaatagaaattagctcttttgagccactggtggggtttaaattcccagcccttatgtgctttgcagctgttagttcctcttattacaattgtctatttaaaaacctagtcacagcccggtgcagtagctcacgtctgtaatcccaacactttgggaggccaaggcaggagaactgcttgagctcaggcgttcaacatcagcctaggcaacatagtgagaccctctcatctctacaaaaagcaaaaaattagccagtgatgcatggctgtagtcccagctattctgagggctgaagttggaggattgcttgagcccaggaggtcaaggctgcagtgggcagtgatcatgccgctgcactctagcctggatgacagagcaagaacctgtctccaaaaaaagaaaggaaggaaggaaggaaggaaggaaggaggaaagaaaagaaagaaagaaagaaagaaagaaagaaagaaagagagagagagagagagagagagagaaagaaagaaagaaagaaagaaagaaagaaagaaagaaagaaagaaagaaagaaagaaagagaaagaaagaaagaagggagggagggagggagaggagagaaagaaaaaggaaggaaggaaggaagagagagagagagagagaaagacctagtcaccaaaagcaagagattttttaaatgctactattttttgggcatttactaatcatattgctatgctctgcacccaagctaagtaatttaaataaattatctcatgtactcctctaaaactaattactgctgtgtaaatggaggtagaaagaaactaagctttatttctgcctctattgtttctttaacctgccttgcttcctttttcagttgcacctaattggctgtacttttagttttctttaaaactgccttaaatttcaaagactaaagcagcaataactaactgaatatatttatataacatgttatttttgtcatgttgctttccacccctggagacctgctctaaattcacttggacgtttgaggataaatcatgctcactagcagtttctgaaaatgcagtttcactgaaaatgcaggcatccagaaatttagtaagcaacttaaaagaaagtgtaagaatctcctatgtattcattgaaaaataatttgaatttatgcttagaaaaatagaattattattaagaaatcttacacactcatgtttttaaatatcttcactaaggaccaattgtgtatatggtgtaacactgtcctcaaagaacatgccgggagaattgttgcagttaccagagggttaaatttggcaaactcttttttattaacgtgccttttaattatgaaatagcatactcaccttagataaaatttgaaaaccatttttgtaaagtggtacaatattgaagaaagttgataactttcagaccagatttaagcctcaaatctacctctcttttacctggacaactcattagcatttctgaacctcacattttttctataaagtgagaatactatattatagagttgttgtcagttaaatgagaacagtgtctgatcacaactagtcaacaaatgttcacaactcttcccctcctaggaaaagaatctcaaggcagacctgcttcgggtctgctctgtaaagaggtaggaatcctctgctcccggtaaattgcttcctaaccttctttggtaatagactattttttaataaaggtgatggatcatttcccattatacactcaaaatgtgtgtccatttcagggcagtcatggatgaccattgcccatcttttgaccccagattaagaacacctgctgtagtattttaattctgccttcaaatcctcttacaaaacaaagacatctttaaaaaataaaattctttaggtgtcttgcagttgaatgcaggaaaaccagagccccttatttttgatagttttgggaagaatgcagtgtcagaacacaaacccataatagacaaataatttgcacagaaacttcataaaagtattgacctgatttgccatgtatttgccaccttttaaaacacacaactaaatgtttaccctgtgtctagatccaaatgggtgaagaaaaatgagtgacaatacatctacttaagctcacttacataattgtggccatgccgtttttttcacattacattattagaacattggacaataagtcaagaaacagaatgttctacaaaataaactttaaaaattggtaagcatcatgtgctttttccagaagacattttattttgttgaatcaaaggtggctctttggcactgagtagctccgtggagtcatggcagtcctcattccctaatcctgagcctgcctgagtcgctgctgtcagtcatccacttgttgggatttcaaactgcattaaatcccctcctatagctgtcactgccaagcagttgcactggctctgtcctacctttctgttggtaattctgtttttaatcctgtgcttcagtgtagtttatataaatctttacagagggataaaacttcctgtaattaattgtttgggtgaacatgtacctgggagagctattgggaaaggggccaaatttgcattccagctcctttcatccccacccttgagctaaccaagtcctgtggattcttcccttagcatctctggaaccttcttttcttttcttttttttatgaccacctttccagtcctggcccttcaaacttgagtgacagcaacagtctccctgccttgagtctctttcctccttctcccagtgtgcatacggttgtcaaactcatcttgataaactactgcatcgattgtggctacactcccctgctcccacatcttccatagaccccactgtctgtaaaataatattcagtctggcctcaacctgtctttccagcctcggtgacacaggtctattctgcctgagacacttactatgacacccttgcttgttcctggggctttgacacatttccaacgtcccattgttcttcctctccaaatcagccaattgcccaagccctgctcaaatctcccacctcatgaagccttcttgatgcctcccagcacaccatgatctaatttcctgaagtaattatgctaattgggcatttgaagaattgttaaccgattatcaactaactgccccttaacattgcatgtgtagttgtcttcaaaggcagttaaattatgtcatgttccttacattgtactgagtgcctcgtatccttatccatgtttgggggttttactttaagtcaagaaatttaatcacatccatttggttttctctagagctgtagttctcaaccttttgtgtggtagagaaacacctagagaacatgtttaaaaatatcctgggttccacccttgagagataataaggtccaaggggaacccaaatatctgtgtttcaggtcagcttattggctcatcctattataccaactcctcagaaggccaaggtgggtggattccttgatctcaggcgttcaagaccagcctgggcaatatcgtgagactccatctcttaaaaaaaaaaaaaaaaaggattagccaagtgtggtggcatgaacctgtggtcccagctacttaagaggctgaggcagacagattgcttgagcctgggaagtcgaagctgcagtgagccatgatcatgccactgcactccagcctgggtgacagagcaagaccctgtctcaaaaaaataaaaatgaaaaaaatctgtgttcccaagttccaagtgatgctgatgctgctggttgcctttaagcatctcacaaagaacgaactcataaatgctaatacagtatatgtctatggatactgaatagtgggttttttttctcttttcttctattctgtgctcatgttgtgtcacttcttccttttagattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcatcagccgtgtctattccatctacgtccacaccgtctgtgacccactctttgaagctgttgggaaaatattcagcaatgtccgcatcaacttgcagaaagaaatataaatgacatttcaaggatagaagtatacctgattttttttccttttaattttcctggtgccaatttcaagttccaagttgctaatacagcaacaatttatgaattgaattatcttggttgaaaataaaaagatcactttctcagttttcataagtattatgtctcttctgagctatttcatctatttttggcagtctgaatttttaaaacccatttaaatttttttccttacctttttatttgcatgtggatcaaccatcgctttattggctgagatatgaacatattgttgaaaggtaatttgagagaaatatgaagaactgaggaggaaaaaaaaaaaaaagaaaagaaccaacaacctcaactgcctactccaaaatgttggtcattttatgttaagggaagaattccagggtatggccatggagtgtacaagtatgtgggcagattttcagcaaactcttttcccactgtttaaggagttagtggattactgccattcacttcataatccagtaggatccagtgatccttacaagttagaaaacataatcttctgccttctcatgatccaactaatgccttactcttcttgaaattttaacctatgatattttctgtgcctgaatatttgttatgtagataacaagacctcagtgccttcctgtttttcacattttccttttcaaatagggtctaactcagcaactcgctttaggtcagcagcctccctgaagaccaaaattagaatatccatgacctagttttccatgcgtgtttctgactctgagctacagagtctggtgaagctcacttctgggcttcatctggcaacatctttatccgtagtgggtatggttgacactagcccaatgaaatgaattaaagtggaccaatagggctgagctctctgtgggctggcagtcctggaagccagctttccctgcctctcatcaactgaatgaggtcagcatgtctattcagcttcgtttattttcaagaataatcacgctttcctgaatccaaactaatccatcaccggggtggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccgtctgttatgctgtgacacatggcccctccccctgccaggagctttggacctaatccaagcatccctttgcccagaaagaagatgggggaggaggcagtaataaaaagattgaagtattttgctggaataagttcaaattcttctgaactcaaactgaggaatttcacctgtaaacctgagtcgtacagaaagctgcctggtatatccaaaagctttttattcctcctgctcatattgtgattctgcctttggggacttttcttaaaccttcagttatgatttttttttcatacacttattggaactctgcttgatttttgcctcttccagtcttcctgacactttaattaccaacctgttacctactttgactttttgcatttaaaacagacactggcatggatatagttttacttttaaactgtgtacataactgaaaatgtgctatactgcatactttttaaatgtaaagatatttttatctttatatgaagaaaatcacttaggaaatggctttgtgattcaatctgtaaactgtgtattccaagacatgtctgttctacatagatgcttagtccctcatgcaaatcaattactggtccaaaagattgctgaaattttatatgcttactgatatattttacaattttttatcatgcatgtcctgtaaaggttacaagcctgcacaataaaaatgtttaacggttaaacagtcagctttattattttttcccaaaacaggtgtttatgtgtcagagtctgtgtatgtctatgtatttgtatgtaatgagcatgtgcatagtgtgtgtatgtgtttgtatgtgtttgtggggggtaatggtctcccactttaaaattattacaaagtcacttaggatatttctgctaaggtcatcaccatttatgagttgcttcagataaaagttataattaataacaaagtttttttagcaatttgcccaatgttttatatgtcatctaatttgagcccccagcaagcttgtgtgatggatattaatactcttaacttagcgaaagacacaatttgcattcggggccaatgccttcaactttgccatgccttaactgggttttaaagaggtatattgcagtctcaatttatgtttgttgcttggctaagtttaccttcaggactcctatattagggttctccagagaaacaaaaccaataggagatagttggagatagatagatagatgatagatgatagatagatagatgatagatagatagatagatagacagatgatagatagatgatagatagatgatagatacatagatagatagaatagagatgataaagatagagagatgaagatagagatagagatggacatggagatggagatagagagatgaaaatatatatatagagagagagatggagatagagatatatagggacagatagacacagagatagaaatagagatagagatagatggagatagagatagagatatatagagacagatagaaatagagatatatagagatggagctacagatagagatagatggagatgatgagaaagaggtagatggagggataaagatatagatggagatgacaggggtagagatagagatagatggagatgatagagatacagagcaagagctttattacaaggaactggcttacacgattatggaggctgacaagttcccaaatctgcagggtgagtcagcaagctgggaacccaggagagctgatgatgtagttccagtccaacatcagcaggctcaagagccaggaaaagctgctattttagaccaagtccaaaggcaggaaaaaaattcaatgttccagtttgaaggcagtcaagcagaaggaattctctcttagttggtggtcagggtcagggtcagcttattctatgcaagccttcaactgattagatgaggcccacccagattagggagggcaatctgccttactccgtctatcagtttaaatgttaatcttatccaaaagcaccctcaaagaaacgttcagaataatatgtgaccaaacatatggacaccccatgacccagtcaagttgacacaaaaagtcaatcatcacagcttccagttccatctacaaaaataactatatggctttggacaacttttattccatattggtaataaatagcttcatacatcacacatttagcctgtagtcctagcagtttggaagcccaaggctgagactgggggatcaattgaggccaggagttcagaccagccttgacaacgtagtgagaccatcagaaaaaagaaaagaaaggaaaggaaagaaaggaaaggaagaaagaaagaaagaaaaagaaagaaagaaaagaaagaaagaaaggaagaaagaaagagagaaagaaagaaagaaaaagaaagaaaagagagaaagaaagaggaaggaagagaaagaggaaggaaggaagaggaaggaagagaaagaaagaggaaggaaggaaggaaggaaggaaggaaggaaggaaggaaggaaggaaggtaggtctcataccttccctgatgtgggtgctaatggtcaagcattctatgttttaatttataatccatatttttaacattgggtggaggggagaagtaaagagagacactcttaacacagaaggctgaaatcataaaataaaaaggtcatggcaataaacacacaaaatatcaaacttctatatgtcaaaaacagtagaaacaaatacaaacaaatgataaagactgataacagaaagcatttgaatgcatagcattataatatacctaaaaataaacagagctttcataaattaagaaaaagatgaataataagccaagatagttcacaaaaagaagtacacaacaattatgcaaaaacttatactcattagtaaagaagtagtaagagttaaagcaatgagacatcattttcacttaagtctgcagagacggtaaacaaaagtaatgtccatgctggtgaagatgcaggcgagtgggcacactcatttattgcatctgtgagggtaactggtataaaccatttggaagacaattaaatgtacacatgatatatcccgcaattcctcttactaaataaaaataatagaggcaataccaaggttcatcatcataaatgttcatcattttattgtcaatagcataatatattggaaacaacataaatattaaaacaaccgaattggttaaataaattatggcatatgcatttaacagacttctattagcttttaaatattggatggaatagaatatttaatgacatggaaatatgttcatagcatgatgttaacttaaatagtaaaatttaaaaatcgtgtgtaggatatactgctactttttgtttaaaagttgggtgtgagtggatttaaagaattgtgtcatttctgagctcctgtcagatactttacagacattattgcaaatttatacaagatttcaaaatagatattgatggtcccattttacaaataaggtttaaagggttttagaaagtcacccattcaccatcattaataagttttggagctattgtctaatccaggggtgtctgtcctcaaagctctgttccttacattcatctatcctcctgctctgtgcggtttctgagccctaagcctttctcctcttcatgaagcttcctgttcccactggcttgcagaattatttcatccactctgaaatgtataataagcaagtaatgtgagcagaactggagtaaatattagcagaagttaagtaatagttaaaacttgtattcaacttcgtatgtcaagtgtatcctttataaatgtattagtttatgtaatatttgcaataaccctatatggtatgtattatcattatcattccaattttacagagtaagaaatcgaggcacagaagagtcaaataactcgctcagagtcacacagctagtaactaagtcaggattatgaacccatggagtctggctccagaatctaggagcttaactatggcaccaaatcatatagttagagggaatcccgcaaaacagaaagcaagttgtgtctctactgagaacaagtaatggtcaacaatatgggagacaattaaaatgcaaattaaaacattttaagtctaaaatataaaattaaaactgctcccaaaaatttaacaaaatcttttatttaagcatttacatataagtttttacgacataaaaaatgtttcataataggatatattcagtcacattttagtttagtgactttcaagaccataaaatgactaagtgataagttgatgtacagactgcaagacagagattttaatgatggaaagaaacacactttttgaaataaactctaatatttatattatttgacaaatattaaataatctccatggactcagattaggttctatcaagtaactgatagaaccattttaccattaagattcaagattttttttctttaggactctccattcatttatttgactttcatattcattctcagatctgccagttgctttagtaataatgaatctcaagacgttgtgcattactcaacttaccatcaatgcaaactctctccacttccccaatgcacaccaatcctactaatacttgctctcatcttatttttctctttcactaaaatggatgattccctactctgagcaaaagccattcccatcacatgcgctctgaatctcatcctttcttattttcacaagatctttgctccatttgtcatcttgtttaactcttttactatctatctctccctttgctctgaattgctctcattaccctccaaacatcgcgtataactttataattttaaaaaaacacattccctcccttaaccttgtaacctcctccaaccatcaatattttttctcctttcttttacagccaaaattaacaaaagaattatttatatacattgtctaaacactattaaattaccctacctataattatacttttaagccaaagaggttttggggtttttgggtttgttttttttttttctgagtcttgataagttgccccatctggtcttgaacacaggggctcaagcaatccactcacctcagcctcctgagaagctgaagctacaggcacatacaacggcgcccagctgaacaggttttttaaacaatttttatttctggtacccagacgtaagcataggcatgctcaatagttaatgtcatcattggtccatgaggggctctcttctatttgattttatttattttattatttaatgatcattaattaacacctatgaattcatcatcaaatcaaagaactagaactaacaactacacatttgttccttctctttcccatccactgtacccctgcatccatatgaggtaaccattctcttaaattttgttatttattatctttttgcctattaaaaaattttaaatacaatctcccaggggaaaaaaaaaacctatttagttttagttctttttaaaatatttctaaggagtaccatgcagaatggatcgttctggtgtttgatttttttttttttcacttaagattcattcatattgttgaatatagctctaagtcattcattttcagtgctatatatttgtctgttccatgaacatatccatttttatccagctgcctcttgagggggacatggtatgccaatggtgctgctataatcatacttctgcatgtccactgggagggtttgtgggcataaattcctctagggattatcattaggactgaattgttcagtcccaaaaaatgatacacccccagcttttctagatatgaccaatggcttttcaaattaataatagcaatttacactgccaccagcattttgaactgtcaaacttcttcaattttgctaatcaagtgggtgtgaaatagtatcttattttagtcttgatttatacctttctgattaccaatgaggttaagcgtcccttcactggtttttggtcatatgagcttcttctgtgaaatgacatgtcattcagcaagggttttttaaaaatgattttttttttcactgagtcataggaattcttaccgattcttaatgttttcattttatttttacatatgtttcaaatatttttctcaatttgtatccttattttcactttcattattaaaatgtctattagtgaacacaagtattttgactttagtgttcatatttatccattttttatttttagtactttttataatgtcaaagagatattttcctgccaagatcagtaagatatttattgtattttaactagcattactgtggtttaagagtttaaatatcttaggtttttctagcataaaaatattttgcaatatgttcaatcacattaaatatttgcttttaacatttaagtccttaatccagttacatggagttgatttgtttgtgttgcatgatgtaggaatccaatttcatttttttctgaataagacaaccatttatgctagtttcattaattgaaaatgtcctcctttctcctttaaactataatgccacctctgttatttattgaagttccattcatacttggattgatttctgaactctctattctagcccactggtcagtttgtttttcctgtactcatatagcattttaaagtgagtctccaaatcttgtaagacaagttttaatttcctgttttctttgctccttactttctaagggccttggttttgtttgt

SEQ ID NO: 36 - DNA, 2876 bp, Homo sapiens
gaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagc

SEQ ID NO: 37 - DNA, 6728 bp, Homo sapiens
agcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccccgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggaccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgccaaaggcgatgctggtccccctgggcctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccccggtcccatgggcccccctggattggctggaccccctggtgaatctggacgtgagggggctcctgctgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcccgtcggccccgtcggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggaagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctcaagggctccaacgagatcgagatccgcgccgagggcaacagccgcttcacctacagcgtcactgtcgatggctgcacgagtcacaccggagcctggggcaagacagtgattgaatacaaaaccaccaagtcctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaacccccccaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaaaaagaataaataaataagtttttaaaaaaggaagcttggtccacttgcttgaagacccatgcgggggtaagtccctttctgcccgttgggttatgaaaccccaatgctgccctttctgctcctttctccacaccccccttggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcatcccttgtgccgcaggctgggcgggagagactgttctgttctgttccttgtgtaactgtgttgctgaaagactacctcgttcttgtcttgatgtgtcaccggggcaactgcctgggggcggggatgggggcagggtggaagcggctccccatttttataccaaaggtgctacatctatgtgatgggtggggtggggagggaatcactggtgctatagaaattgagatgcccccccaggccagcaaatgttcctttttgttcaaagtctatttttattccttgatattttttctttctttttttttttttttgtggatggggacttgtgaatttttctaaaggtgctatttaacatgggaggagagcgtgtgcgctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccctaggggtgggaggaagcaaaagactctgtacctattttgtatgtgtataataatttgagatgtttttaattattttgattgctggaataaagcatgtggaaatgacccaaacataatccgcagtggcctcctaatttccttctttggagttgggggaggggtagacatggggaaggggccttggggtgatgggcttgccttccattcctgccctttccctccccactattctcttctagatccctccataaccccactcccctttctctcacccttcttataccgcaaacctttctacttcctctttcattttctattcttgcaatttccttgcaccttttccaaatcctcttctcccctgcaataccatacaggcaatccacgtgcacaacacacacacacactcttcacatctggggttgtccaaacctcatacccactccccttcaagcccatccactctccaccccctggatgccctgcacttggtggcggtgggatgctcatggatactgggagggtgaggggagtggaacccgtgaggaggacctgggggcctctccttgaactgacatgaagggtcatctggcctctgctcccttctcacccacgctgacctcctgccgaaggagcaacgcaacaggagaggggtctgctgagcctggcgagggtctgggagggaccaggaggaaggcgtgctccctgctcgctgtcctggccctgggggagtgagggagacagacacctgggagagctgtggggaaggcactcgcaccgtgctcttgggaaggaaggagacctggccctgctcaccacggactgggtgcctcgacctcctgaatccccagaacacaacccccctgggctggggtggtctggggaaccatcgtgcccccgcctcccgcctactcctttttaagctt

SEQ ID NO: 38 - DNA, 441 bp, Homo sapiens
ggccctcggggcccaaggaattcgcaagatggcgccgtccgccttgctgcgtcccctttcccggctgctggcccccgccaggctcccgagcggcccttcagtgcgatcaaagttctacgtgcgagagccgccgaatgccaaacctgactggctgaaagttgggttcaccttgggcaccactgtcttcttgtggatctatctcatcaaacaacacaatgaagatattttagagtacaaaagaagaaatgggctggaataaacttttgaaacactaatgtagtatgctccgtatagtgattgtagctgttcctctggattcaccatctgttgagttgtaaatgtgagagaaaaagttatatgtgaatatatatcaagccagcatttgtattttgcatcattaaataaaaagaaataaaaatacttctgtattcttcaaaaaaa

SEQ ID NO: 39 - DNA, 1888 bp, Homo sapiens
accgctagcccgcagcgctcggcttcctggtaattcttcacctcttttctcagctccctgcagcatgggtgctgggccctccttgctgctcgccgccctcctgctgcttctctccggcgacggcgccgtgcgctgcgacacacctgccaactgcacctatcttgacctgctgggcacctgggtcttccaggtgggctccagcggttcccagcgcgatgtcaactgctcggttatgggaccacaagaaaaaatagtagtggtgtaccttcagaagctggatacagcatatgatgaccttggcaattctggccatttcaccatcatttacaaccaaggctttgagattgtgttgaatgactacaagtggtttgccttttttaagtataaagaagagggcagcaaggtgaccacttactgcaacgagacaatgactgggtgggtgcatgatgtgttgggccggaactgggcttgtttcaccggaaagaaggtgggaactgcctctgagaatgtgtatgtcaacacagcacaccttaagaattctcaggaaaagtattctaataggctctacaagtatgatcacaactttgtgaaagctatcaatgccattcagaagtcttggactgcaactacatacatggaatatgagactcttaccctgggagatatgattaggagaagtggtggccacagtcgaaaaatcccaaggcccaaacctgcaccactgactgctgaaatacagcaaaagattttgcatttgccaacatcttgggactggagaaatgttcatggtatcaattttgtcagtcctgttcgaaaccaagcatcctgtggcagctgctactcatttgcttctatgggtatgctagaagcgagaatccgtatactaaccaacaattctcagaccccaatcctaagccctcaggaggttgtgtcttgtagccagtatgctcaaggctgtgaaggcggcttcccataccttattgcaggaaagtacgcccaagattttgggctggtggaagaagcttgcttcccctacacaggcactgattctccatgcaaaatgaaggaagactgctttcgttattactcctctgagtaccactatgtaggaggtttctatggaggctgcaatgaagccctgatgaagcttgagttggtccatcatgggcccatggcagttgcttttgaagtatatgatgacttcctccactacaaaaaggggatctaccaccacactggtctaagagaccctttcaacccctttgagctgactaatcatgctgttctgcttgtgggctatggcactgactcagcctctgggatggattactggattgttaaaaacagctggggcaccggctggggtgagaatggctacttccggatccgcagaggaactgatgagtgtgcaattgagagcatagcagtggcagccacaccaattcctaaattgtagggtatgccttccagtatttcataatgatctgcatcagttgtaaaggggaattggtatattcacagactgtagactttcagcagcaatctcagaagcttacaaatagatttccatgaagatatttgtcttcagaattaaaactgcccttaattttaatatacctttcaatcggccactggccatttttttctaagtattcaattaagtgggaattttctggaagatggtcagctatgaagtaatagagtttgcttaatcatttgtaattcaaacatgctatattttttaaaatcaatgtgaaaacatagacttatttttaaattgtaccaatcacaagaaaataatggcaataattatcaaaacttttaaaatagatgctcatatttttaaaataaagttttaaaaataactgcaaaaaaaaaaaaaaaaaaa

SEQ ID NO: 40 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 41 - DNA, 408 bp, Homo sapiens
agtaacagccaagatgctgcggaatctgctggctcttcgtcagattgggcagaggacgataagcactgcttcccgcaggcattttaaaaataaagttccggagaagcaaaaactgttccaggaggatgatgaaattccactgtatctaaagggtggggtagctgatgccctcctgtatagagccaccatgattcttacagttggtggaacagcatatgccatatatgagctggctgtggcttcatttcccaagaagcaggagtgacttcagtcatcccagcaatcgcttggttcagtttcattcagctctctatggaccagtaatctgataaataaccgagctcttctttggggatcaatatttattgacttgtagtaactgccaccaataaagcagtctttaccatg

SEQ ID NO: 42 - DNA, 4326 bp, Homo sapiens
gctgagcgcggagccgcccggtgattggtgggggcggaagggggccgggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatagagcatgaatttttttcatcttctctggcgacagttttccttctcatctgtgattccctcctgctactctgttccttcacatcctgtgtttctagggaaatgaaagaaaggccagcaaattcgctgcaacctgttgatagcaagtgaatttttctctaactcagaaacatcagttactctgaagggcatcatgcatcttactgaaggtaaaattgaaaggcattctctgaagagtgggtttcacaagtgaaaaacatccagatacacccaaagtatcaggacgagaatgagggtcctttgggaaaggagaagttaagcaacatctagcaaatgttatgcataaagtcagtgcccaactgttataggttgttggataaatcagtggttatttagggaactgcttgacgtaggaacggtaaatttctgtgggagaattcttacatgttttctttgctttaagtgtaactggcagttttccattggtttacctgtgaaatagttcaaagccaagtttatatacaattatatcagtcctctttcaaaggtagccatcatggatctggtagggggaaaatgtgtattttattacatctttcacattggctatttaaagacaaagacaaattctgtttcttgagaagagaatattagctttactgtttgttatggcttaatgacactagctaatatcaatagaaggatgtacatttccaaattcacaagttgtgtttgatatccaaagctgaatacattctgctttcatcttggtcacatacaattatttttacagttctcccaagggagttaggctattcacaaccactcattcaaaagttgaaattaaccatagatgtagataaactcagaaatttaattcatgtttcttaaatgggctactttgtcctttttgttattagggtggtatttagtctattagccacaaaattgggaaaggagtagaaaaagcagtaactgacaacttgaataatacaccagagataatatgagaatcagatcatttcaaaactcatttcctatgtaactgcattgagaactgcatatgtttcgctgatatatgtgtttttcacatttgcgaatggttccattctctctcctgtactttttccagacacttttttgagtggatgatgtttcgtgaagtatactgtatttttacctttttccttccttatcactgacacaaaaagtagattaagagatgggtttgacaaggttcttcccttttacatactgctgtctatgtggctgtatcttgtttttccactactgctaccacaactatattatcatgcaaatgctgtattcttctttggtggagataaagatttcttgagttttgttttaaaattaaagctaaagtatctgtattgcattaaatataatatgcacacagtgctttccgtggcactgcatacaatctgaggcctcctctctcagtttttatatagatggcgagaacctaagtttcagttgattttacaattgaaatgactaaaaaacaaagaagacaacattaaaacaatattgtttctaattgctgaggtttagctgtcagttctttttgccctttgggaattcggcatggtttcattttactgcactagccaagagactttacttttaagaagtattaaaattctaaaattcaaaaaaaaaaaaaaaaaa

SEQ ID NO: 43 - DNA, 822 bp, Homo sapiens
ataaaaatggcttccaaaagagctctggtcatcctggctaaaggagcagaggaaatggagacggtcatccctgtagatgtcatgaggcgagctgggattaaggtcaccgttgcaggcctggctggaaaagacccagtacagtgtagccgtgatgtggtcatttgtcctgatgccagccttgaagatgcaaaaaaagagggaccatatgatgtggtggttctaccaggaggtaatctgggcgcacagaatttatctgagtctgctgctgtgaaggagatactgaaggagcaggaaaaccggaagggcctgatagccgccatctgtgcaggtcctactgctctgttggctcatgaaataggttttggaagtaaagttacaacacaccctcttgctaaagacaaaatgatgaatggaggtcattacacctactctgagaatcgtgtggaaaaagacggcctgattcttacaagccgggggcctgggaccagcttcgagtttgcgcttgcaattgttgaagccctgaatggcaaggaggtggcggctcaagtgaaggctccacttgttcttaaagactagagcagcgaactgcgacgatcacttagagaaacaggccgttaggaatccattctcactgtgttcgctctaaacaaaacagtggtaggttaatgtgttcagaagtcgctgtccttactacttttgcggaagtatggaagtcacaactacacagagatttctcagcctacaaattgtgtctatacatttctaagccttgtttgcagaataaacagggcatttagcaaactaaaaaaaaaaaaaaaaaaa

SEQ ID NO: 44 - DNA, 1134 bp, Homo sapiens
cgctaagattgctacctggactttcgttgaccatgctgtcccgggtggtactttccgccgccgccacagcggccccctctctgaagaatgcagccttcctaggtccaggggtattgcaggcaacaaggacctttcatacagggcagccacaccttgtccctgtaccacctcttcctgaatacggaggaaaagttcgttatggactgatccctgaggaattcttccagtttctttatcctaaaactggtgtaacaggaccctatgtactcggaactgggcttgtcttgtacgctttatccaaagaaatatatgtgattagcgcagagaccttcactgccctatcagtactaggtgtaatggtctatggaattaaaaaatatggtccctttgttgcagactttgctgataaactcaatgagcaaaaacttgcccaactagaagaggcgaagcaggcttccatccaacacatccagaatgcaattgatacggagaagtcacaacaggcactggttcagaagcgccattacctttttgatgtgcaaaggaataacattgctatggctttggaagttacttaccgggaacgactgtatagagtatataaggaagtaaagaatcgcctggactatcatatatctgtgcagaacatgatgcgtcgaaaggaacaagaacacatgataaattgggtggagaagcacgtggtgcaaagcatctccacacagcaggaaaaggagacaattgccaagtgcattgcggacctaaagctgctggcaaagaaggctcaagcacagccagttatgtaaatgtatctatcccaattgagtcagctagaaacagttgactgactaaatggaaactagtctatttgacaaagtctttctgtgttggtgtctactgaagttatagtttacccttcctaaaaatgaaaagtttgtttcatatagtgagagaacgaaatctctatcggccagtcagatgtttctcatccttcttgctctgcctttgagttgttccgtgatcattctgaataagcatttgcctttataaaaacttgctgcctgactaaagattaacaggttatagtttaaatttgtaattaattctaccatcttgcaataaagtgacaattgaatg

SEQ ID NO: 45 - DNA, 2091 bp, Homo sapiens
gagcggagccgcgggcgggagggcggacggaccgactgacggtagggacgggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgcaggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttcttccccaacccctcagattttatattttctatttctctgtgtatttatataaaaatttattaaatataaatatccccagggacagaaaccaaggccccgagctcagggcagctgtgctgggtgagctcttccaggagccaccttgccacccattcttcccgttcttaactttgaaccataaagggtgccaggtctgggtgaaagggatacttttatgcaaccataagacaaactcctgaaatgccaagtgcctgcttagtagctttggaaaggtgcccttattgaacattctagaaggggtggctgggtcttcaaggatctcctgtttttttcaggctcctaaagtaacatcagccatttttagattggttctgttttcgtaccttcccactggcctcaagtgagccaagaaacactgcctgccctctgtctgtcttctcctaattctgcaggtggaggttgctagtctagtttcctttttgagatactattttcatttttgtgagcctctttgtaataaaatggtacatttct

SEQ ID NO: 46 - DNA, 1579 bp, Homo sapiens
accaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaacaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactagaaaaaaaaaaaaaaaaaaaaaaaaaaaaaaaaaaaaaaaaaaaaaaaaaaaaaaaaaaaaaaaaa

SEQ ID NO: 47 - DNA, 693 bp, Homo sapiens
tccccgcccttctcttttctacagtatctgttacctcagtcagattgaggatgcacatatactgggaaaataatcgacttccagcgtccctcatgtagtctctacctgattcaggaatctctaacccagtatcggaacgttaagcggcttctccgctttcctgccgtgagaaactcgaggtgaccaacctccgtttccggttggctccggttgcagagttgagtgtcctgagaggtcagattgctgtcagacatggcccatgaacatggacatgagcatggacatcataaaatggaacttccagattatagacaatggaagatagaagggacaccattagaaactatccagaagaagctggctgcaaaagggctaagggatccatggggccgcaatgaagcttggagatacatgggtggctttgcaaagagtgtttccttttctgatgtattctttaaaggattcaaatggggatttgctgcatttgtggtagctgtaggagctgaatattacctggagtccctgaataaagataagaagcatcactgaagataatacctggaagcatcatagtggtttcttaactctccaaaataagatttcttctctgtagcctacttgtctggtttatcccttacagaatattagtaaagatttaatcaattaaaatatatataaaaaaaaaaaaaaaaa

SEQ ID NO: 48 - DNA, 3420 bp, Homo sapiens
actcaccaccatcttggtagtgtgcaagactctgggacggcatgagaaaggcagaaagggagcaaaggagctggtggggtgtcacagggcctatgtatgaggcagatgtgagttctgcatccagttttgcaatttatccatttacctgcctgagacttggaaacacctactttatagtcctgattcaagtgttggcgatggaagggaggagactcctgaccctcagaatgacctaaatgacttcacaagggcccgtgtaacaccagtattgcagacccatgtgtcacaaattcaactagcacattgtttttctcccccttcttctatttctgtagaagatgaatccgcaggcaagaatgatggatgggtgaaagggagggggaatacgaaagcccagaggcggagacttcacttccgattgccacgctatatcaagaaagccaacagtctggcctttccgcaagccacaagaaaaggcagatcagactccccggctcctcgggtagttctcaaaggcaaagacaaaaataagaaactagtctgaagaaagagagtccctcgcttcttcagaactcacctgtcaagagcggcgtggacggcgccatgttgtcccggaaagagaaaccgctcgggtgagagttcaccagggccgaacccggcccctacacgtctcctcatgcgtggagacagcgaattggtgtttccggagccatcagcctcggggccaattcacttccggttacggagtgacgcggaaaaggcgcccgaacgcgcacgcgcaaatctagggcgacgcttgacagagcttgggagggtgcgcctgctttcgccctccttctccagcgggaggggcgcgcacttccgcggggcggagtccgtctagtgctgacgttggcagccgaacccaaagtagatcgaggcggcgggctgcacattcccgttgttgcgttgcgtttccttcctctttcactccgcgctcacggcggcggccaaagcggcggcgacggcggcgcgagaacgacccggcggccagttctcttcctcctgcgcacctgccctgctcggtcagtcagtcggcggccggcgcccggcttgtgctcagacctcgcgcttgcggcgcccaggcccagcggccgtagctagcgtctggcctgagaacctcggcgctccggcggcgcgggcaccacgagcggagcctcgcagcggctccagaggaggcaggcgagtgagcgagtccgaggggtggccggggcaggtggtggcgccgcgaagatggtcgccaagcaaaggatccgtatggccaacgagaagcacagcaagaacatcacccagcgcggcaacgtcgccaagacctcgagaaatgcccccgaagagaaggcgtctgtaggaccctggttattggctctcttcatttttgttgtctgtggttctgcaattttccagattattcaaagtatcaggatgggcatgtgaagtgactgaccttaagatgtttccattctcctgtgaattttaacttgaactcattcctgatgtttgataccctggttgaaaacaattcagtaaagcatcctgcctcagaatgactttcctatcatgcttcatgtgtcattccaaggtttcttcatgagtcattccaagttttctagtccataccacagtgccttgcaaaaaacaccacatgaataaagcaataaaatttgattgttaagatacagtagtggaccctacttattcagtcaattaagagtaagtttttttatgtggttattaaaacagtatgaacaattagtctaactctgcatagacagggtctagattttgttaacccaaatgtataactgcagttagcttaaattacaatttgaagtcttgtggtttttatatagctaggcactttattactcttttgaactgaaagcacactcccttataggttcatgtaactgtcctgtaataaggtgcttataaatggaacaactacacagcctagttttgccacaacctttagcatctaaaaagttttaaaagcttctaaatgtctaatataaagggagatgcttatagccacaacatctattttaccaatattgtttccattacactaccttggattttgcatgagtgagtatagtaacccaagatgccataaaaaaaaaacttgatcgttttctgacttaatcagttactgtggtttcactaaaagctaccgtggtggagtgaagtcagtcagggaaggtttgtttatgttacatttatttcaccagaactattttaatatatcaaaggggtttactatgccaaacaaaattctagggaaaaatactgctaaaaatggatgcctcatcagaacatgctgttgagtccaatgtgccataagacattttagcatgttaaatagcacttttaatagcaaaaaaaggcacatcaactgcgaagttatccttagtttgcaaatgctttttctagattaatgatttttcaatcattagggtactagacacatcagcctaaagtggcatctggaattgaatggatttactgataatgatcagtctttagtcttccctttgttatatgactttataggttatgattgatcaaatttacgttttactaatggtaagggtgagggtcatagggcaggttttgggttttctagtactgttgaaaactgcaagtattggctatttgtatacttagccataacttggtgaaaaaaaacctgagcagtgtctatgtattaatgcgttggaaagaaagctgcttgtgtttgctttgttaattgcctcaggatatttcttttaaaataagctgttttaagaggaacagaagggaaatctgctacctagtctatacacagcatgaacctcacagggggcttctgataccctcaaacatggagaacagtaagggagcagagtggttaaggactttcaggaacttaactattctggaataaggaatgaatcaactgaccttgggccagcaggtttttaactaaattgttacttgcctttctcacccagttaatcagtctctgtacttgtttccctttttgaaacaagtgtcttggttaactaattctgttttatggttgtgctaaattcatagcaggtgccttattctttgcttttagtcaaaccattccatatcagaattttccttggtttactatagatatttggctttaagttgttgtttgtgttttttaatgtacaatgttctgataaatttgactgttaaattgctatagctagcaatcattttacatatgtaaaattgcattccctttgtatttcatgtgtaattcaccaattaagtgcagtttatattcaggttggattatgcatgtttaggtaaacgaaagctgtgtcttacttgatttattctttaaaaataaagttccctgaatatttg

SEQ ID NO: 49 - DNA, 987 bp, Homo sapiens
gagcagcggacgccggtctctgttccgcagatggggtttgttaaagttgttaagaataaggcctactttaagagataccaagtgaaatttagaagacgacgagagggtaaaactgattattatgctcggaaacgcttggtgatacaagataaaaataaatacaacacacccaaatacaggatgatagttcgtgtgacaaacagagatatcatttgtcagattgcttatgcccgtatagagggggatatgatagtctgcgcacgttatgcacacgaactgccaaaatatggtgtgaaggttggcctgacaaattatgctgcagcatattgtactggcctgctgctggcccgcaggcttctcaataggtttggcatggacaagatctatgaaggccaagtggaggtgactggtgatgaatacaatgtggaaagcattgatggtcagccaggtgccttcacctgctatttggatgcaggccttgccagaactaccactggcaataaagtttttggtgccctgaagggagctgtggatggaggcttgtctatccctcacagtaccaaacgattccctggttatgattctgaaagcaaggaatttaatgcagaagtacatcggaagcacatcatgggccagaatgttgcagattacatgcgctacttaatggaagaagatgaagatgcttacaagaaacagttctctcaatacataaagaacagcgtaactccagacatgatggaggagatgtataagaaagctcatgctgctatacgagagaatccagtctatgaaaagaagcccaagaaagaagttaaaaagaagaggtggaaccgtcccaaaatgtcccttgctcagaagaaggatcgggtagctcaaaagaaggcaagcttcctcagagctcaggagcgggctgctgagagctaaacccagcaattttctatgattttttcagatatagataataaacttatgaacagcaactaaaaa

SEQ ID NO: 50 - DNA, 502 bp, Homo sapiens
agattccgtcgcttcttccggagccgtacgtggcaccgccccgctcgcgggcggccgcggggcttgctgggaagagaggcgaagccaggtcacctttcaaggacccagaagtagggttttggcctaggtaacggggcagagatgtggttcgagattctccccggactctccgtcatgggcgtgtgcttgttgattccaggactggctactgcgtacatccacaggttcactaacgggggcaaggaaaaaagggttgctcattttgggtatcactggagtctgatggaaagagataggcgcatctctggagttgatcgttactatgtgtcaaagggtttggagaacattgattaaggaagcattttcctgattgatgaaaaaaataactcagttatggccatctacccctgctagaaggttacagtgtattatgtagcatgcaatgtgttatgtagtgcttaataaaaataaaatgaaaaaaatgcaaaaaaaaaaaaaaaaa

SEQ ID NO: 51 - DNA, 5947 bp, Homo sapiens
tttctcccattctacttccctcctcagcattggaagctcgtaagtgggctctgactcccagcctacagagagattccttaagaagtggttcgactgataaacgcattgccaaaagtgaactgggatgaggtccaagacatctgcggtgggggttctccagaccttagtgttcttccactacaaagtgggtccaacagagaaaggtctgtgttcaccaggtggccctgaccctggagagtccagggcagggtgcagctgcattcatgctgctggggaacatgccctcaggttactcaccccatgacatgttggccccagggactgaaaagcttaggaaatggtattgagaaatctggggcagccaaaaggggagaggccatggggagaagggggggctgagtgggggaaagcaggagcgagataaaaagccagctccagcaggcgctgctcactcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacctccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtcagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgccgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtctttgggtgccccaactgaccacacccccattccatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagag

SEQ ID NO: 52 - DNA, 578 bp, Homo sapiens
cctcctgggagggagctgaagccgctcgcaagactcccgtagtccccacctctctcagcttccggctggtagtagttccgcttcctgtccgactgtggtgtctttgctgagggtcacattgagctgcaggttgaatccggggtgcctttaggattcagcaccatggcggaagacatggagaccaaaatcaagaactacaagaccgccccttttgacagccgcttccccaaccagaaccagactagaaactgctggcagaactacctggacttccaccgctgtcagaaggcaatgaccgctaaaggaggcgatatctctgtgtgcgaatggtaccagcgtgtgtaccagtccctctgccccacatcctgggtcacagactgggatgagcaacgggctgaaggcacgtttcccgggaagatctgaactggctgcatctccctttcctctgtcctccatccttctcccaggatggtgaagggggacctggtacccagtgatccccaccccaggatcctaaatcatgacttacctgctaataaaaactcattggaaaagtgaaaaaaaaaaaaaaaaaaaaa

SEQ ID NO: 53 - DNA, 448 bp, Homo sapiens
caaggtcgtgaaaaaaaaggtcttggtgaggtgccgccatttcatctgtcctcattctctgcgcctttcgcagagcttccagcagcggtatgttgggccagagcatccggaggttcacaacctctgtggtccgtaggagccactatgaggagggccctgggaagaatttgccattttcagtggaaaacaagtggtcgttactagctaagatgtgtttgtactttggatctgcatttgctacacccttccttgtagtaagacaccaactgcttaaaacataaggatgtttcagttcctccatttaacagatatgaagagcattttaagaggtgcagcctctggaagtggatcaaactagaactcatatgccatactagatatgtttgtcaataaacttatgacgtgaaaaaaaaaaaaaaaaaaaaaaaaaaaaaaaaaaaaaaaaaaa

SEQ ID NO: 54 - DNA, 404 bp, Homo sapiens
gtggacagggtcatcctgagggtgcgactccgccgcgatggtgacccggttcctgggcccacgctaccgggagctggtcaagaactgggtcccgacggcctacacatggggcgctgtgggcgccgtggggctggtgtgggccaccgattggcggctgatcctggactgggtaccttacatcaatggcaagtttaagaaggataattaattacacaaacccttcacagactgctctggtgcctggtggtgctagctcctcccacctcagcacctgctgcatctggagcagcccaagctctcaggatggacaagaggaaacccacagctcagcttcaggcttcttatgtttctgaaaacagcttggatattttaatgcacgttgcattaaacctcactgaaacctg

SEQ ID NO: 55 - DNA, 1892 bp, Homo sapiens
ctcacgggagtcctccgcccgggcgtccggaacagtcgacggcagactccggcccgctgagccacccgaggggtcccgtggcctccgcggacccggaatctgggccctcgcggacccgcgccccgcccagtcgccccagcccgtccactgctctgttcgccagggcttccccacacccacggagtgaagtcagccgcggccctgcctgggaggaacttaccgtctaccgggaaaggtggccagcagatgtgtcgggcctggtgagaggggcctcctgcactggccatgcccatgcccacggagggcaccccgccacctctgagtggcacccccatcccagtcccagcctacttccgccacgcagaacctggattctccctcaagaggcccagggggctcagccggagcctcccacctccgccccctgccaagggcagcattcccatcagccgcctcttccctcctcggaccccaggctggcaccagctgcagccccggcgggtgtcattccggggcgaggcctcagagactctgcagagccctgggtatgacccaagccggccagagtccttcttccagcagagcttccagaggctcagccgcctgggccatggctcctacggagaggtcttcaaggtgcgctccaaggcggacggccggctctatgcggtaaagcgttccatgtcaccattccggggccccaaggaccgggcccgcaagttggccgaggtgggcagccacgagaaggtggggcagcacccatgctgcgtgcggctggagcaggcctgggaggagggcggcatcctgtacctgcagacggagctgtgcgggcccagcctgcagcaacactgtgaggcctggggtgccagcctgcctgaggcccaggtctggggctacctgcgggacacgctgcttgccctggcccatctgcacagccagggcctggtgcaccttgatgtcaagcctgccaacatcttcctggggccccggggccgctgcaagctgggtgacttcggactgctggtggagctgggtacagcaggagctggtgaggtccaggagggagacccccgctacatggcccccgagctgctgcagggctcctatgggacagcagcggatgtgttcagtctgggcctcaccatcctggaagtggcatgcaacatggagctgccccacggtggggagggctggcagcagctgcgccagggctacctgccccctgagttcactgccggtctgtcttccgagctgcgttctgtccttgtcatgatgctggagccagaccccaagctgcgggccacggccgaggccctgctggcactgcctgtgttgaggcagccgcgggcctggggtgtgctgtggtgcatggcagcggaggccctgagccgagggtgggccctgtggcaggccctgcttgccctgctctgctggctctggcatgggctggctcaccctgccagctggctacagcccctgggcccgccagccaccccgcctggctcaccaccctgcagtttgctcctggacagcagcctctccagcaactgggatgacgacagcctagggccttcactctcccctgaggctgtcctggcccggactgtggggagcacctccaccccccggagcaggtgcacacccagggatgccctggacctaagtgacatcaactcagagcctcctcggggctccttcccctcctttgagcctcggaacctcctcagcctgtttgaggacaccctagacccaacctgagccccagactctgcctctgcacttttaaccttttatcctgtgtctctcccgtcgcccttgaaagctggggcccctcgggaactcccatggtcttctctgcctggccgtgtctaataaaaagtatttgaaccttg

SEQ ID NO: 56 - DNA, 2482 bp, Homo sapiens
tcttttgccaaccacttgaagaagtctgcgaaaacgccaaaaaatgcagttcgaaatgacagtgccatttcgctattgcctattttgtccttgattttattctctggcacttttgtctttaatcgtcgtatttttccatctcttgttctcatacccttctactctatttccgtcttacacaagttctttaaccttagtttatttctttgtcagaatcgccaagaagccagttatcaaatagcgagccttctccccccaccttttttttttttttaaattactgtactgatgtttgttagatattgttcgaatagacctggggtggacatcagcactgccattgtcagccctgtgagggttagaagagcttggtgcatattaaacactgaacagatgttagctgttgttgtcttgagcaagttttcttaccattctgggccttaatttctttttgtttaagatggaagtaatacctaatttttaggttgtgagaattatttgaaatgtgaaaaatattgtcagtgggtctgagccgtagtaaacagtaaatagcagctggttacagtagtatccccttaatggggtaggtatacgttccaagacccccagtggatggctgaaaccacggatagcaccaaaccctatatatacttggttttttcctataccgtacatacatacctacctatgataaaacttacaagttaagcacaataagagattaacaataacataatagaactgtaacaatatactataataaaagttatgtgaatatgatctctttccccttctctcaaaataacttattttactggactcacctattttcacaccagattgaccgcggataattgaaaccggaaagtggaacctcggataagggggtgattactgtatcaccctttaatatgccatctgttagtattcgtatcaccttgtagtcatgtgtagctttcttctacccattctgcaaaattgctttacatcaagtatttactattctttttagcagtttttatccccttctgagttttgttagggattctcttgaggtatatgcaattagctgaagacctactttaatattggattagaatgtttctcctgttttcacacttacaggtttctctggtgtttactcgtttctttgtggaattgcagtttcacctatgcacacacctgtattctttctctttaagaagttacgtgaattgtgtatgttctgttggaaggatggtaaaacttttatctttattcctagaaaaaatgtagaagatttcactggacctagagaaagaagtgatctgggatttatcacatttgatataactgctgatctagagaatatatttgattggaatgttaagcagttgtttctttatttatcagcagaatattcaacaaaaaataatgctctgaaccaagttgtcctatgggacaagattgttttgagaggtgataatccgaagctgctgctgaaagatatgaaaacaaaatattttttctttgacgatggaaatggtctcaagggaaacaggaatgtcactttgaccctgtcttggaacgtcgtaccaaatgctggaattctacctcttgtgacaggatcaggacacgtatctgtcccatttccagatacatatgaaataacgaagagttattaaattattctgaatttgaaacaacatatttttatacttaatgaattgtatctcattaatctcttcccttacatcttcatgtattgttggtttgttttttggttttgggttttttttttttttttttggtataagaactaacatcaaaaggcctgtttaaagggaaaggttaatgggctacttaatattatgaacaaaacaaaaaaacaaggctgccacagtggaatattatcttacaagaataagaactacataaaacagatttgtaaaaaatacatatttgaagtattccctgtatttccattattctttatggaatataaagtaagcatgaaaggtagttaaaactttcaggtgcctgtagagttataataactgtattttatgccttgcattcacgcaaattcacattggatgtgatttaaaagtagacattctctttttcctcttttaggatatgtttgattattggaaaattaatatggttatttgttagaagtctggtttataaaaaagccaaaagtgatggaatttattccatttgtcttaggaaggcccataatacttgtttttcttacatgtgactagcaactttctccacttaaagactaaatacctctttatatgatgtaaattattctaattcattttaaaatcttttaggtcagcaaaatgtgtgtcttcagtgctttctctaaaaacgttcctttatagctttgtacttttttttttagcgttgccattgaaagttaaaatgtttgcatggtttaccctctgagttatgtttcttctagtaagcctctacctgcacgacaacacat

SEQ ID NO: 57 - DNA, 1645 bp, Homo sapiens
gcgggctggcgtgcggcgccgttgcgggcgggagcggctgcaacgccggtgcctgaggagcgatgccgagggaaatcatcaccctacagttgggccagtgcggcaatcagattgggttcgagttctggaaacagctgtgcgccgagcatggtatcagccccgagggcatcgtggaggagttcgccaccgagggcactgaccgcaaggacgtctttttctaccaggcagacgatgagcactacatcccccgggccgtgctgctggacttggaaccccgggtgatccactccatcctcaactccccctatgccaagctctacaacccagagaacatctacctgtcggaacatggaggaggagctggcaacaactgggccagcggattctcccagggagaaaagatccatgaggacatttttgacatcatagaccgggaggcagatggtagtgacagtctagagggctttgtgctgtgtcactccattgctggggggacaggctctggactgggttcctacctcttagaacggctgaatgacaggtatcctaagaagctggtgcagacatactcagtgtttcccaaccaggacgagatgagcgatgtggtggtccagccttacaattcactcctcacactcaagaggctgacgcagaatgcagactgtgtggtggtgctggacaacacagccctgaaccggattgccacagaccgcctgcacatccagaacccatccttctcccagatcaaccagctggtgtctaccatcatgtcagccagcaccaccaccctgcgctaccctggctacatgaacaatgacctcatcggcctcatcgcctcgctcattcccaccccacggctccacttcctcatgaccggctacacccctctcactacggaccagtcagtggccagcgtgaggaagaccacggtcctggatgtcatgaggcggctgctgcagcccaagaacgtgatggtgtccacaggccgagaccgccagaccaaccactgctacatcgccatcctcaacatcatccagggagaggtggaccccacccaggtccacaagagcttgcagaggatccgggaacgcaagttggccaacttcatcccgtggggccccgccagcatccaggtggccctgtcgaggaagtctccctacctgccctcggcccaccgggtcagcgggctcatgatggccaaccacaccagcatctcctcgctcttcgagagaacctgtcgccagtatgacaagctgcgtaagcgggaggccttcctggagcagttccgcaaggaggacatgttcaaggacaactttgatgagatggacacatccagggagattgtgcagcagctcatcgatgagtaccatgcggccacacggccagactacatctcctggggcacccaggagcagtgagtcccccaggacagggaccctcatctgccttactggttggcccaagccctgcctgactgaccaccccctcagagcacagatcagggacctcacgcatctctttctcatatacatggactctctgttggcctgcaaacacatttacttctcctcttatgagactatttatctttaataaagcactggatataaaaaaaaaaaaaaaaaaaaaaaaaaaaaaaaaaaaa

SEQ ID NO: 58 - DNA, 3698 bp, Homo sapiens
gcaagatggacgcgggatttttccgcggaacaagtgcagaacaggataatcggttcagcaacaaacagaagaaactactgaagcagctgaaatttgcagaatgcctagaaaaaaaggtggacatgagcaaagtaaatttggaggttataaagccttggataacaaaaagagtaacggaaatccttgggtttgaagatgatgttgtgattgagtttatattcaaccagctggaagtgaagaatccagactccaaaatgatgcaaatcaacctgactggatttttgaatggaaaaaatgctcgagaatttatgggagaactgtggcccctgctgctaagtgcacaagaaaacatcgcgggaatcccttctgctttcctagaactgaagaaagaagaaataaaacaaagacagattgaacaagaaaaactggcatctatgaaaaagcaagatgaagacaaagataaaagagataaggaagaaaaagaaagcagcagagaaaaaagggagcggtctcgtagcccaagaagacgcaaatccagatctccttcccctagaagacgatcttcccctgtcaggagagagagaaagcgcagtcattctcgatctccccgtcacagaaccaagagccggagtccttcccctgctccagaaaagaaggaaaaaactccagagctcccagaaccttcagtgaaagtaaaagaaccttcagtacaagaggctacttctactagtgacattctgaaagttcccaaacctgaacctataccagagcctaaagaaccttctccggaaaaaaattccaaaaaagaacaggagaaggagaagacccgaccacgatctcggtcacgctccaaatcaagatcccggacgcggtcccgctctccttctcacactcgacctagacggcgccatagatccgataagatgtattcacctagaaggcggccaagcccaagaaggcggccatctcctcgaagaagaactccgccaagaagaatgcctcctccaccaaggcatagaaggagtagatctccagtaagacgaagaagacgttcgtcagcatccttgtctgggagtagctcatcatcctcttcatctcgttcacggtcaccaccaaagaagcctcccaagaggacatccagcccccctcggaaaactcgtaggttatctccttcagcaagtcctccaaggcgaaggcacaggccatcacctcctgcaacaccacccaaaactcgggattcccctacaccccagcagtcaaaccgtacaagaaaaagtcgtgtttctgtgtctccagggagaacttcaggtaaagtgacaaaacataaaggtactgagaaaagagaatccccttcaccagcaccgaagcctagaaaagtagagttatctgaatcggaagaagataaaggtggcaaaatggctgcagcagattctgtgcagcagagacgccaatacagacgacaaaaccagcagtcttcatctgactctggctcctcctcctcctcagaagatgaacgacccaagagatcccatgtgaagaatggtgaggttggcaggcggcggagacattccccttcccggagtgcttctccatcaccacgaaagcgccaaaaagagacttcccctcgtggtagacggaggagaagtccatccccaccacccaccagaaggcgacggtctccttctcccgcccctcctcctcgacggcgcaggactcccacaccaccaccacgacgaaggactccttctcctcccccacgtcggcgctcaccttctcctagaagatactctcctccaatacagaggagatactctccttctccacctccaaagagaagaacggcttcacctcctccccctcctaaacgaagagcatcaccatctccaccaccaaagcggcgggtctcccattctccacctcccaaacaaagaagctccccagtcaccaagagacgttcaccttcattatcatccaagcataggaaagggtcttccccaagccgctctacccgggaggcccgatcaccacaaccaaacaaacggcattcgccctcaccacggcctcgagctcctcagacctcctcaagtcctccacccgttcgaagaggagcgtcgtcatcaccccaaagaaggcagtccccgtctccaagtactaggcccattaggagagtctccaggactccggaacctaaaaagataaaaaaggctgcttccccaagcccacagtctgtaagaagggtctcatcctcccgatctgtctccgggtctcctgagccagcagctaaaaagcccccagcacctccatcccccgtccagtctcagtcaccgtctacaaactggtcaccagctgtaccggtcaaaaaggccaaaaagcccaacaccgagcccatcaccgccaagaaattcagatcaggaaggaggtggaaagaaaaagaagaaaaagaaggacaagaaacacaaaaaggataagaagcacaagaagcacaaaaaacacaagaaggaaaaggctgtggctgcagctgctgcagctgctgtgacccctgcagccattgcagctgccacaaccacattagcacaggaagagccagtggcagcgccagagccgaagaaggagactgaaagtgaagctgaagataaccttgatgatttagaaaagcacctgcgtgaaaaggccctgagatcaatgaggaaggcccaagtgtccccacagtcttagggggaaatgtttgttatgatgtaaattttatttggtttgtacgcagttcaatttcaaaattgctaaaatgtgtttgagctttagactataacatttgttgtaataattgctaggttgaagttcaacatgtaaaaaaagggggcatggatttacattgcaaaaggtgtccacagtgtattagtgacattctttcattgacagctgacataattcattgagtgaaatattttaagccaaaaaaaaattccctttttaaaaaagggggtttaaatactgttggcatttttatggttcctttaaatgccctagaattcccagaggggttttttgtttgtttttttggttttgattttctttttgtttttctttcttcttcttatttttttcatttgagtcttagctcccatttaagttatgcttctgaccttgtatggtctgtaagcttgcccagaaataagaccactgttttgaactaccacaaaagtataaatgaatattttaatgccacaatctttcctgttgcctgtggagtctctgctgaaatgaatcaggattcgagctctaggatgagacagaaaatgaaagcatgttgtttgccaggacactgtgggtttatattgatgtgtaacaagttgatttggaacactggactctcattctgttattctggcttcgccccttttgttccccctccttcttttgtaaaggcaatgagctagtcccagaaaggatccttcagttacatacaatttatttaatgaaatgtcatggctctgttcatatttttgtcttgttcttccaattggtatatacaactttcagagcctcttgtatttggaaggctggaagggcccagactttggaatagtgtcttggtttcactgtttttgttttgattttttttttgttttgattttttttaaactaaagctatataaagcttgtggattaaacagaataaatttctaaatttaaaaaaaaaa

SEQ ID NO: 59 - DNA, 1601 bp, Mus musculus
gcaccaggaaagtgacccgggtgtagaatcgtccgcccagaacttcccgttggctgcactggggcgacacctatgcagtcgcccaaggacacagagcacggacagtcaagctcagggctacttagagggaaactctgaaagttgaaatctgaaggtcagcagccaggacagatgacaagtgaacaaggctgaagtcaggtgtgggcgcagagtggattcaaaggagtttgcctctgcagggaagacagcattaaggaatgaaaagggagatcgccggcttctacgggcaccacgtgtccacctcccgtatctattgagcatgctgacatccgggtcaagaattacagtgtgaactccagggagaggagacccctccctagctcactacagtccagtgccaacagtagtgacaccaaaggtgacctcacagccagagagcccctccccctctgcaaaagagccagaagctttctctcccaaatcagataccgcaatgaccacagagacagctattatgcctggctccaggctgacaccatcccaaacaacttctgcaggaactacagggacaggcagtcacaagtcctcccgagccccatctcttgcagcaacaatgaccttggagcctacagcctccacctccctcaggataacagagatttctccccacagttccaaaatgacgaaagtggccatctctacatcggtcctcttggttggtgcaggggttgtgatggctttcctggcctggtacatcaaatcaaggcagccttctcagccgtgccgtgttgaggtggaaaccatggaaacagtaccaatgactgtgagggccagcagcaaggaggatgaagacacaggagcctaaccacacgatttcagaaactcagggagacctgcccagctgatcctgaagagaagatacaagggcagttagtccagctgcgcctggacacaggagaaagcccaatgtgataatagaggtctctggtatgatctgttttactgagcctgggtgcctcccatattcaggcctggatccccggagtccggaaggacaccacctttcttcctggttcatcacagaggccaacttcccagagtacaagcagcctgagctctcctgggagtctccattaaaagcctcgagttccactcagagattaactcaaagcagctgatctgctagactctttttctattccctactctgacttgcagtttacagagattgacaaggctcccattgtcttccaaggctcctctggcacaggagatgtctgtaaagaagacagcaacatttgagctcctgaagacttggccctttgctgctttgcacctattggaggagagcagagaacagaagaagagatactgagccaatgaaccctttcgtataggattcatgacaaaaccaaactcagtgactatatatgtatgtgtgtgtgtgtgtgtatgtaaaagtgtatatttacatatacatttatatttatactttcttttctattatatctacatattgtatatgatttatatttgaaagtgctttgtgtagacaaaataaaatatctattttcagtacaaaaagcattaaaattatcaac

SEQ ID NO: 60 - DNA, 710 bp, Homo sapiens
aagctttgtgcagggcaggaacggtaggccagactggggcaacctcaaggacacacggagtccaaagggttgtttacatactgtgtcctgcaccccaagcaaaacatctcctggagaacagggccagggctcaatctccccacaaaggcaggggcgtggagagaagcgacctctactttcccatccctcgcctcaattctgcacccgatctagctatggggtcaagcctggaggggacgctggttctccagacatggtcccgcccactgcaagtgccaccttggtaagggatttctccgagcagaggcaaacgcgaatttgtggccttcttaatcaagatgaggtcttcgagccctgtctgccgcgccaggtcgcgttaccttcttatgctcctccgccacagccagaccacgaccctcctgcacgcaatcccaccgagactgcgccttttccccgcaccccagcaggtccctaagtaagatggcggcgaacgcacttccggcgtgtgtccttacgggtgcgtccgggcggctgcttgcaggtgccacccagcgggttccagcttgtttgctgcatagattacaacggtgatggcgggcaagcggtccggctggagccgggcggctctcctccagctccttctcggcgtgaacctggtggtgatgccgcccacccgggcccggagtctgcgcttcgttaccttggtaatg

SEQ ID NO: 61 - DNA, 5144 bp, Homo sapiens
ccctgtccctggctcacgtgatcgcgcctagggagaaaacgtctgactccagccaccggccttcaaggcacggctttttattccttcggctggtcggcctctcgcccttcagctacctgtgcgtccctccgtcccgtcccgtcccggggtcaccccggagcctgtccgctatgcggctcctgcctctagccccaggtcggctccggcggggcagcccccgccacctgccctcctgcagcccagcgctgctactgctggtgctgggcggctgcctgggggtcttcggggtggctgcgggaacccggaggcccaacgtggtgctgctcctcacggacgaccaggacgaagtgctcggcggcatgacaccgctaaagaaaaccaaagctctcatcggagagatggggatgactttttccagtgcttatgtgccaagtgctctctgctgccccagcagagccagtatcctgacaggaaagtacccacataatcatcacgttgtgaacaacactctggaggggaactgcagtagtaagtcctggcagaagatccaagaaccaaatactttcccagcaattctcagatcaatgtgtggttatcagaccttttttgcagggaaatatttaaatgagtacggagccccagatgcaggtggactagaacacgttcctctgggttggagttactggtatgccttggaaaagaattctaagtattataattacaccctgtctatcaatgggaaggcacggaagcatggtgaaaactatagtgtggactacctgacagatgttttggctaatgtctccttggactttctggactacaagtccaactttgagcccttcttcatgatgatcgccactccagcgcctcattcgccttggacagctgcacctcagtaccagaaggctttccagaatgtctttgcaccaagaaacaagaacttcaacatccatggaacgaacaagcactggttaattaggcaagccaagactccaatgactaattcttcaatacagtttttagataatgcatttaggaaaaggtggcaaactctcctctcagttgatgaccttgtggagaaactggtcaagaggctggagttcactggggagctcaacaacacttacatcttctatacctcagacaatggctatcacacaggacagttttccttgccaatagacaagagacagctgtatgagtttgatatcaaagttccactgttggttcgaggacctgggatcaaaccaaatcagacaagcaagatgctggttgccaacattgacttgggtcctactattttggacattgctggctacgacctaaataagacacagatggatgggatgtccttattgcccattttgagaggtgccagtaacttgacctggcgatcagatgtcctggtggaataccaaggagaaggccgtaacgtcactgacccaacatgcccttccctgagtcctggcgtatctcaatgcttcccagactgtgtatgtgaagatgcttataacaatacctatgcctgtgtgaggacaatgtcagcattgtggaatttgcagtattgcgagtttgatgaccaggaggtgtttgtagaagtctataatctgactgcagacccagaccagatcactaacattgctaaaaccatagacccagagcttttaggaaagatgaactatcggttaatgatgttacagtcctgttctgggccaacctgtcgcactccaggggtttttgaccccggatacaggtttgacccccgtctcatgttcagcaatcgcggcagtgtcaggactcgaagattttccaaacatcttctgtagcgacctcacacagcctctgcagatggatccctgcacgcctctttctgatgaagtgattgtagtaggtgtctgtagctagtcttcaagaccacacctggaagagtttctgggctggctttaagtcctgtttgaaaaagcaacccagtcagctgacttcctcgtgcaatgtgttaaactgtgaactctgcccatgtgtcaggagtggctgtctctggtctcttcctttagctgacaaggacactcctgaggtctttgttctcactgtattctttttatcctggggccacagttcttgattattcctcttgtggttaaagactgaatttgtaaacccattcagataaatggcagtactttaggacacacacaaacacacagacacaccttttgatatgtaagcttgacctaaagtcaaaggacctgtgtagcatttcagattgagcacttcactatcaaaaatactaacatcacatggcttgaagagtaaccatcagagctgaatcatccaagtaagaacaagtaccattgttgattgataagtagagatacattttttatgatgttcatcacagtgtggtaaggttgcaaattcaaaacatgtcacccaagctctgttcatgtttttgtgaattctaggctggtgctgcactgaaatagagcagtaagcttgtgataaaggccaattccaggtagctcttgaaggtgatagccatctactttccagtggctgccaaccacagggagtgccagttaacactggaaggattaaggcaaggtcccttctcttgagactcccctctgagatctgaaaaatgaagtggcttaggaacatcagcagtgaagaactgccaagagttggtgaaggttgtctcttccgagggccttctgaagacagggctcttgaacagacaagtggaagggctgtaccagggataaaggaaagaagtgcctgtccagcagggagcttgaatttaagttccatgtatgaagtcattggctctatctgcatttttctgtcattctcttcatttgttttaaggtggaaaattttcttacagttgatgcaaagtatcaactactttaccctaccttctccccttttagatgggttcttcctgagttttggagtcttgtatgattatcagtattcccctgtcaaaatcaaatctattcaggtttcttcactgttgagaacacctaaatgtttttatttttgagaagtggggacagagtctcactatgtcacccaggctggagtgcaatggcatgatctcagctcactgcaaccttcgcctcctgggttcaagcgattctcctgcctccgcctcctgagtagctgggattataggcacgcaccaccacgcccagctaattttttgtatttttagtagagacagagtttcaccatgttggccaggctggtcttgaactcctgaccttgtgatccacccacctcggcctcccagagtgctgggattacaggcatgagccaccacgcttggctaagaacacctaaatttttatgtttcttggctcaaaaaccagttccatttctaatgttgtcctcacaagaaggctaattggtggtgagacagcaggggaggaggaagagctgtggtttgtaacttgttcaactcaggcaataagcgattttagctttatttaaagtcttctgtccagctttaagcactttgtaagacatggctgaaagtagcttttctatcagaattgcagatagtcatgttgggctaacagtcaattggatatattcctttacctcacatgaccccagcaactgtggtggtatctagaggtgaaacaggcaagtgaaatggacacctctgctgtgaatgttttagagaaggaaattcaaaaaatgttgtaactgaaagcactgttgaatatgggtatcggctttctttttcactttgactcttaacattatcagtcaacttccacattaatgaaagttgaccatagttatttccaaataaaaagaaaccaactcttaccaggtcttggactgtgatgtcatattattcagttttatgcttgttcctgagcagaactcataagagtgacatagtcagctgctgacggcacctcagccacgccactcttactcagttcagtgggtgtgcttgcgtggtaggatgtggtgcagccctctctacgctcttctatttttggtatatttcctatctaaccttcaaatagcttccaattctttttttcttggactggcttcattctgaatttgtgctaaaataatctttcataaagagacctcagtttatagcgtaacagactacacaatgcactgatgttttcataatgtttaagggacccactgcaagaagcttgctgcctccttttaattgtattcatttagattttgattttccatgttaagaaggtgaggtccatgttggtgcccttcagagtagagaaccatgtaaacattaggaatgaacagaggccttaggaatgaatagagagtttgccttatacaatttcctgttacaaagctctccctctcatgcaaagtagggaacaccttttgagcatctttgaatttgacaaatggtgctgttgcaaacactttttttttgagatgaagtctcgcggttgtcacccgggctggagtgcagtggcgtgatctcggctcactgcaacttccacctcctgggttccagcagttctcctgcctcagcctcccaagtagctgagattacaggcgcctgccaccccacctggctgatttttgtaattttagtagagacggggtttcaccatgttggccaggctgattaactcctgacctcaggtgatccacctttctcggcctcccaaagtgctgggattacgggtgtgagccaccgtgcccggcctgcaaacacattttaattgacaacactagggctgttgtacaaaatagtaatgatagccatggaagttttaccttattctgtgagaagtgttcttaaacttattaagtgtctaaactaaggtttagtgcttttttaaaggaaagttgtcccaggattcatcctaaagaaagcaaaagttaattcaactgatccaccaatggaattagatgggtagagttgggttcttgagttttaccaccacttagttcccactgaattttgtaacttcctgtgtttgcatcctctgttcctattctgcccttgctctgtgtcatctcagtcatttgacttagaaagtgcccttcaaaaggaccctgttcactgctgcacttttcaatgaattaaaatttatttctgttctagtgggaaaaaaaaaaaaaaaaaa

SEQ ID NO: 62 - DNA, 523 bp, Homo sapiens
cggaagttttgctgctagtcgcggacgcaatggcttcaaggttacttcgcggagctggaacgctggccgcgcaggccctgagggctcgcggccccagtggcgcggccgcgatgcgctccatggcatctggaggtggtgttcccactgatgaagagcaggcgactgggttggagagggagatcatgctggctgcaaagaagggactggacccatacaatgtactggccccaaagggagcttcaggcaccagggaagaccctaatttagtcccctccatctccaacaagagaatagtaggctgcatctgtgaagaggacaataccagcgtcgtctggttttggctgcacaaaggcgaggcccagcgatgcccccgctgtggagcccattacaagctggtgccccagcagctggcacactgagcacctgcactaaattactcaaaatgtgctgtaaagtttcttctttccagtaaagactagccattgcattggctccttctcccataaaaaaaaaaaaaaaaaaa

SEQ ID NO: 63 - DNA, 2007 bp, Homo sapiens
tttaataaccatggactccaagtacagcagcaacagcaaaggaatctctcactacatgaatacatgagtatggaattattgcaagaagctggtgtctccgttcccaaaggatatgtggcaaagtcaccagatgaagcttatgcaattgccaaaaaattaggttcaaaagatgtcgtgataaaggcacaggttttagctggtggtagaggaaaaggaacatttgaaagtggcctcaaaggaggagtgaagatagttttctctccagaagaagcaaaagctgtttcttcacaaatgattgggaaaaaattgtttaccaagcaaacgggagaaaagggcagaatatgcaatcaagtattggtctgtgagcgaaaatatcccaggagagaatactactttgcaataacaatggaaaggtcatttcaaggtcctgtattaataggaagttcacatggtggtgtcaacattgaagatgttgctgctgagactcctgaagcaataattaaagaacctattgatattgaagaaggcatcaaaaaggaacaagctctccagcttgcacagaagatgggatttccacctaatattgtggaatcagcagcagaaaacatggtcaagctttacagcctttttctgaaatacgatgcaaccatgatagaaataaatccaatggtggaagattcagatggagctgtattgtgtatggatgcaaagatcaattttgactctaattcagcctatcgccaaaagaaaatctttgatctacaggactggacccaggaagatgaaagggacaaagatgctgctaaggcaaatctcaactacattggcctcgatggaaatataggctgcctagtaaatggtgctggtttggctatggccacaatggatataataaaacttcatggagggactccagccaacttccttgatgttggtggtggtgctacagtccatcaagtaacagaagcatttaagcttatcacttcagataaaaaggtactggctattctggtcaacatttttggaggaatcatgcgctgtgatgttattgcacagggtatagtcatggcagtaaaagacttggaaattaaaatacctgttgtggtacggttacaaggtacacgagtcgatgatgctaaggcactgatagcggacagtggacttaaaatacttgcttgtgatgacttggatgaagctgctagaatggttgtaaagctctctgaaatagtgaccttagcgaagcaagcacatgtggatgtgaaatttcagttgccaatatgatctgaaaacccagtggatggctgaaggtgttaaatgtgctataatcattaagaatactgtgttctgtgttattgttctttttctttttagtgtgtggagattgtaattgccatctaggcacacaaacatttaaaaggatttggactgcatttaattgtaccattcagaatggactgtttgtacgaagcatgtataatgcagttatcttctttcttttgtcgcagccagtcttttttgcttctcctacaaaacgtaacttgcaatttgccagtttattattgttggatacaaagttcttcattgataagagtcctataaataagataaatacgaagataaagctttattctttagtgttaaaatacagtatatctaataactagcctcattagtagagcagtatattaaaacaatgttttatgtaaaaagtgtttatcttcagcaccaaatacatgataaatgtatcaatcactatttataaacagagctttcaaacactcctcagaatattcttctaagtattttgatgaagtaactttgtaattatttgaacattgttttaatcattaggaaacactgattaactgcaagtcttcatgattctgtcatattaagaaacacctgtaggtttgcttcaaataaaggcatatataccaaggacttacagacaaaattaagaatgtcaatttaagttaataaaaatctcccaatatgaaaaaaaaaaaa

SEQ ID NO: 64 - DNA, 2794 bp, Homo sapiens
gggcggagcacttcccctctgctgggcgcgcggtggacggtctgaaagggagtgttcgggtttcgctggggcctcgcggctccagagcccagcatggcttcctcgcgagcctcttccacggcaaccaaaactaaagcacccgacgacttagttgctccggtcgtgaaaaaaccacacatctattatggaagtttggaggagaaggagagggagcgtctggccaaaggagagtctgggattttggggaaagacggacttaaagcagggatcgaagctggaaatattaatataacctctggagaagtgttcgagattgaagagcatatcagcgagcgacaggcagaattattggctgagtttgagagaaggaagcgagcccggcagatcaatgtttccacagatgactcagaggtcaaagcttgccttagagccttgggggaacccatcacactttttggagagggtcctgctgaaagaagagaaaggttaaaaaatatcctctcagttgtcgatactgatgccttgaaaaagaccaaaaaggatgatgagaagtctaaaaagtccaaagaagagtatcagcaaacctggtatcatgaaggaccaaatagcttgaaggtggcaagactatggattgctaattattcgttgcccagggcaatgaaacgcttggaagaggcccgactccataaggagattcctgagacaacaaggacctcccagatgcaagagctgcacaagtctctccggtctttgaataatttttgcagtcagattggggatgatcggcctatctcctactgtcactttagtcccaattccaagatgctggccacagcttgttggagtgggctttgcaagctctggtctgttcctgattgcaacctccttcacactcttcgagggcataacacaaatgtaggagcaattgtattccatcccaaatccactgtctccttggacccaaaagatgtcaacctggcctcttgtgcggctgatggctctgtgaagctttggagtcttgacagtgatgaaccagtggcagatattgaaggccatacagtgcgtgtggcgcgggtaatgtggcatccttcaggacgtttcctgggcaccacctgctatgaccgttcatggcgcttatgggatttggaggctcaagaggagatcctgcatcaggaaggccatagcatgggtgtgtatgacattgccttccatcaagatggctctttggctggcactgggggactggatgcatttggtcgagtttgggacctacgcacaggacgttgtatcatgttcttagaagaccacctgaaagaaatctatggaataaatttctcccccaatggctatcacattgcaaccggcagtggtgacaacacctgcaaagtgtgggacctccgacagcggcgttgcgtctacaccatccctcctcatcagaacttagtgactggtgtcaagtttgagcctatccatgggaacttcttgcttactggtgcctatgataacacagccaagatctggacgcacccaggctggtccccgctgaagactctggctggccacgaaggcaaagtgatgggcctagatatttcttccgatgggcagctcatagccacttactcatatgacaggaccttcaagctgtggatgctggaatagatgacaatgggaaaaggacttgaacctcaagctctctctaaggagctgttttcctcaaacgagaagaattgaagtgttagttctatcatgttttctgccaattaccatgcatagaccctcagtagaattggatttccatgtcagcccccactccaggaaggcagcccaatccctaggtgatggggaacccctctcacggttcaaaatttattacctttttacgccctgccacgaactgtgtagacattgtttttattaatcttttgtttggccgggcgtggtggctcacgcctgtaatcctagcactttgggaggccgaggtgggtagatcgcttgagctcaggagttcaagatgagcctgggcaacatggcaaatgccgtctctgcaaaaaaatactaaaattagctggtcgcggtggcttctgcctgtgattccggctacttgggaggctgaggtgggagggattgcttaagcctgggaggtagaggttgcagtgagccgagattgcgccattgcactctagcctgtgtgacagagcaagaccctgtctcaaaaaaaaaaaaaaatttgttcgaatgccttatagccttcctcacagcacccaggattgtgactgactctgcatttttaattcttgaaacttggctttccataacatggtacatgcttcaggcctacatatgacccagagagcaaggtggctgaactatagtctggaagccctcaggtaaagaggcacatctcaccactcattgcttaaacaattgattcatagcgagcacttttcctttccctggagaatgggatgtgaagcagtagaccgcagccacgccgatggttatacagtgaagaagacttcacctcttcctattgagtttgcttggaatgctgacagctcaggcactctgaactgaacatttgctttgtcagaaaatatctttttttttacctttgaagtttggcaaccttcatgttaccccaaagcaaaaccattgtgtcaggagtcaaacaaatgtttagaaagcaaacatgacgtctctattgtacaacctcctttctcttggctgtttaaaggatgtacttcgtgtattaaagggtactttatgttgagtacgaaaaaaaaaaaaaaaaaaa

SEQ ID NO: 65 - DNA, 2600 bp, Homo sapiens
ggatccaaaggagacctatagtgactcccaggagctcttagtgaccaagtgaaggtacctgtggggctcattgtgcccattgctctttcactgctttcaactggtagttgtgggttgaagcactggacaatgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaacgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgaagagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctaggttcccatatttaagaccagtctttgtctagttgggatctttatgtcactagttatagttaagttcattcagacataattatataaaaactacgtggatgtaccgtcatttgagga

SEQ ID NO: 66 - DNA, 2006 bp, Homo sapiens
gaatgatccacgaactgctcttggctctgagcgggtaccctgggtccattttcacctggaacaagcggagtggcctgcaggtatcgcaggacttccctttcctccaccccagtgagaccagtgtcctgaatcgactctgccggctcggcacagactatattcgcttcactgagttcattgaacagtacacgggccatgtgcaacagcaggatcaccatccatctcaacagggccaaggtgggttacatggaatctacctgcgggccttctgcacagggctggattctgttttgcagccttatcgccaagcactgcttgatttggaacaagagttcctgggtgatccccatctctccatatcacatgtcaactacttcctagaccagttccagcttctttttccctctgtgatggttgtagtagaacaaattaaaagtcaaaagattcatggttgtcaaatcctggaaacagtctacaaacacagctgtggggggttgcctcctgttcgaagtgcactggaaaaaatcctggccgtttgtcatggggtcatgtataaacagctctcagcctggatgctccatggactcctcttggaccagcatgaagaattctttatcaaacaggggccatcttctggtaatgtcagtgcccagccagaagaggacgaggaggatctgggcattgggggactgacaggaaaacaactgagagaactgcaggacttgcgcctgattgaggaagagaacatgctggcaccatctctgaagcagttttccctacgagtggagattttgccatcctacattccagtgagggttgctgaaaaaatcctatttgttggagaatctgtccagatgtttgagaatcaaaatgtgaacctgactagaaaaggatccattttgaaaaaccaggaagacacttttgctgcagagctgcaccgtctcaagcagcagccactcttcagcttggtggactttgaacaggtggtggatcgcattcgcagcactgtggctgagcatctctggaagttgatggtagaagaatccgatttactgggtcagctgaagatcattaaagacttttaccttctgggacgtggagaactgtttcaggccttcattgacacagctcaacacatgttgaaaacaccacccactgcagtaactgagcatgatgtgaatgtggcctttcaacagtcagcacacaaggtattgctagatgatgacaaccttctccctctgttgcacttgacaatcgagtatcacggaaaggagcacaaagcagatgctactcaggcaagagaagggccttctcgggaaacttctccccgggaagcccctgcatctggctgggcagccctaggtctttcctacaaagtacagtggccactacatattctcttcaccccagctgtcctggaaaagtacaatgttgtttttaagtacttactgagtgtgcgccgggtgcaagctgagctgcagcactgctgggccctacaaatgcagcgcaagcacctcaagtcgaaccagactgatgcaatcaagtggcgcctaagaaatcacatggcatttttggtggataatcttcagtactatctccaggtagatgtgttggagtctcagttctcccagctgcttcatcagatcaattctacccgagactttgaaagcatccgattggctcatgaccacttcctgagcaatttgctggctcaatcctttatcctattgaaacctgtgtttcactgcctgaatgaaatcctagatctctgtcacagtttttgttcgctggtcagtcagaacctaggcccactggatgagcgtggagccgcccagctgagcattctcgtgaagggctttagccgccagtcttcactcctgttcaagattctctccagtgttcggaatcatcagatcaactcagatttggctcaactactgttacgactagattataacaaatactatacccaggctggtggaactctgggcagtttcgggatgtga

SEQ ID NO: 67 - DNA, 1682 bp, Homo sapiens
gcaggaccgtcattgacgccatgagcgcgctgctgcggctgctgcgcacgggtgccccagccgctgcgtgcctgcggttggggaccagtgcagggaccgggtcgcgccgtgctatggccctgtaccacactgaggagcgcggccagccctgctcgcagaattaccgcctcttctttaagaatgtaactggtcactacatttccccctttcatgatattcctctgaaggtgaactctaaagaggaaaatggcattcctatgaagaaagcacgaaatgatgaatatgagaatctgtttaatatgattgtagaaatacctcggtggacaaatgctaaaatggagattgccaccaaggagccaatgaatcccattaaacaatatgtaaaggatggaaagctacgctatgtggcgaatatcttcccttacaagggttatatatggaattatggtaccctccctcagacttgggaagatccccatgaaaaagataagagcacgaactgctttggagataatgatcctattgatgtttgcgaaataggctcaaagattctttcttgtggagaagttattcatgtgaagatccttggaattttggctcttattgatgaaggtgaaacagattggaaattaattgctatcaatgcgaatgatcctgaagcctcaaagtttcatgatattgatgatgttaagaagttcaaaccgggttacctggaagctactcttaattggtttagattatataaggtaccagatggaaaaccagaaaaccagtttgcttttaatggagaattcaaaaacaaggcttttgctcttgaagttattaaatccactcatcaatgttggaaagcattgcttatgaagaagtgtaatggaggagctataaattgcacaaacgtgcagatatctgatagccctttccgttgcactcaagaggaagcaagatcattagttgaatcggtatcatcttcaccaaataaagaaagtaatgaagaagagcaagtgtggcacttccttggcaagtgattgaaacatctgaaattctgctgtcaagattcccatctctaaggactccaagtgctagagacaagggggtctatgagcatttactgacttcctgttaaaacttcattttttcaaactttttgagctatgcaatatataaataaacagtaagaattttaaattactctcctttggacatttattgatataaaaagaagattttggccaggcacggtggctcatgcctgtaatttcagcactttgggaggccgaggtgggcagatcacaaggtcaggagttcaagaccagcctggccagtatgttgaaaccccatctctattaaaaatacaaaaattagccaggcgtggtggtgcatgcctgtagacccagctactcaggaggctgaggcagaagaactgcttgagcccaggaggcagaggttgcagtgagcagagattgtgccactgcactccagcctaggcgacagagagagactccgtctcaaaaaaaaaaaaaagattttgtttcatttcatagcacaaatttagaggcattgagttgaccatttctagtttctatacctgaaggaacccattgattttaccagtttttatttctgtagattggatgtaaaaatcacagattcaataaattaataaactttgtgtaaaaaaaaaa

SEQ ID NO: 68 - DNA, 1944 bp, Homo sapiens
ccaagatgggcgccatcttgcggtccttttcctggggccggtggtctggtaccctaaatctctcagtattgttgcccttggggctgcgtaaggcccactcgggcgctcaggggttactggcagcgcagaaggctcgaggtctgttcaaggacttcttcccggagacggggacgaaaatagagctcccagagctcttcgaccgtggcacggcgagttttccccaaaccatttactgtggcttcgaccccacggcagactcgcttcatgtgggtcatctacttgcgctgctgggtctgtttcatttgcagcgagcgggccacaacgtgatcgcgctggtgggaggcgccacggcggccctgggagacccgagcggccgtaccaaggaacgcgaggcgctggagacagagcgcgtgcgagccaacgcgcgagctctgcgcctagggcttgaggccctggcggctaatcaccagcagcttttcactgatgggcgctcctggggcagcttcactgtgctggacaactcggcctggtaccagaagcagcacctggtggacttcctggcggcagtgggggttcacttccgcatggggacgctgctgagccggcagagcgtgcagctgcggctcaagagccccgagggcatgagcttggccgagttcttttaccaggtgctccaggcctatgacttctattacctcttccagcgttatggatgcagggtccagctgggcggatctgatcaactaggcaacatcatgtccggatatgagttcatcaacaagttgactggagaagatgtatttggaatcaccgttactctaattacaagtacaactggagcaaagctgggaaagtctgctggcaacgctgtttggctaaacagagataagacatctccatttgaattgtatcaattctttgtcagacaacccgaggattcagtggaaaggtacctgaagctgttcactttcctgcccctcccagagattgatcatatcatgcagctgcatgtcaaagagccagaaaggcggggtcctcagaaacgactggcagcagaagtaacaaagcttgttcatggacgagaaggattggattctgctaaaaggtgtacacaagccctttatcacagtagcatagatgcactggaggtcatgtctgatcaggagttaaaagagttgtttaaagaagctccattttctgaattttttctcgatcctggaacaagtgtcctagatacttgccgcaaagcaaatgccattccagatggtccccgagggtatcgaatgataacagaaggcggagtcagcataaatcaccaacaagtaacaaatcctgagagtgttttaattgttggacaacatattctcaagaatggactttccttacttaaaataggaaaaagaaatttctacattataaaatggcttcagttgtgatgaaaagtccttctggttgtccaaataaacttacccatcattcattctcaagacctctgaagggttggctccagaacttagacctttgcttatgcaaatcagaaaaacagaatggactaggactcagtgtgagtaacttcattatttttatgggccggttaataaatatttgtttaataatgggatgttttattttctgatgtacaaagcttgattaccatagaaaaccatgattttcagggttaatctctttttaaaattatacttcagaagagaaagaaatgctgctttcctatcttacccttcctgctctcctttttgtagtgatgaggaacaatgaaaaagaggtagtgtaaggaattgtgaggctgggcatggtgtaagtgctccactttagagagtgtaccttaattactactgtatttgcaaattaagggaaatttaggtcatcatttgattttcggaaatctaaaaggaactgaccatcattaccaagtctttctatata

SEQ ID NO: 69 - DNA, 1266 bp, Homo sapiens
atgttccgagcggcggctccggggcagctccggcgggcggcctcattgctacgatttcagagtaccctggtaatagctgagcatgcaaatgattccctagcacccattactttaaataccattactgcagccacacgccttggaggtgaagtgtcctgcttagtagctggaaccaaatgtgacaaggtggcacaagatctctgtaaagtagcaggcatagcaaaagttctggtggctcagcatgatgtgtacaaaggcctacttccagaggaactgacaccattgattttggcaactcagaagcagttcaattacacacacatctgtgctggagcatctgccttcggaaagaaccttttgcccagagtagcagccaaacttgaggttgccccgatttctgacatcattgcaatcaagtcacctgacacatttgtgagaactatttatgcaggaaatgctctatgtacagtgaagtgtgatgagaaagtgaaagtgttttctgtccgtggaacatcctttgatgctgcagcaacaagtggcggtagtgccagttcagaaaaggcatcaagtacttcaccagtggaaatatcagagtggcttgaccagaaattaacaaaaagtgatcgaccagagctaacaggtgccaaagtggtggtatctggtggtcgaggcttgaagagtggagagaactttaagttgttatatgacttggcagatcaactacatgctgcagttggtgcttcccgtgctgctgttgatgctggctttgttcccaatgacatgcaagttggacagacgggaaaaatagtagcaccagaactttatattgctgttggaatatctggagccatccaacatttagctgggatgaaagacagcaagacaattgtggcaattaataaagacccagaagctccaattttccaagtggcagattatggaatagttgcagatttatttaaggtagttcctgaaatgactgagatattgaagaaaaaatgaatcaggatcatgccttaaaaagaaaacttttgttaaagtattccactgaaatcacagatatttgtgggtattataacaatcattggaaagcatggagagctacatttcataatttgagggaaaatttctaacagatgccagaatgcttgtttatgggattgctgtgtttccttttaattatttgtggttccaaacaattattgtttgaacttttttaattctgtactaaaatctataataaagcttttccacagctttaaaact

SEQ ID NO: 70 - DNA, 1105 bp, Homo sapiens
ccgacccgagctccagtagtccgcccgctggtcatcgcgccctttcccctgccggtgtcctgctcgccgtccccgccatgctgtctctagactttttggacgatgtgcggcggatgaacaagcggcagctctattatcaagtcctaaattttggaatgattgtctcatcggcactaatgatctggaaggggttaatggtaataactggaagtgaaagtccgattgtagtggtgctcagtggcagcatggaacctgcatttcatagaggagatcttctctttctaacaaatcgagttgaagatcccatacgagtgggagaaattgttgtttttaggatagaaggaagagagattcctatagttcaccgagtcttgaagattcatgaaaagcaaaatgggcatatcaagtttttgaccaaaggagataataatgcggttgatgaccgaggcctctataaacaaggacaacattggctagagaaaaaagatgttgtggggagagccaggggatttgttccttatattggaattgtgacgatcctcatgaatgactatcctaaatttaagtatgcagttctctttttgctgggtttattcgtgctggttcatcgtgagtaagaagcctgccttgctgttcctgggaagatgccatagttttcgttactggatgtttggagtagatactggtctgtgattggtggaatggagaacacacgtgttggtgcttctgggtagcactggtttgcattagtttatgtttccatgccagagtttgtgtgggcgggcgcatgtgcaccacagagtgcactcgaggggactttcagtcacaggatttcataattgtcattgtcacactttcaaatttttgtacatcagtgaatttttttatattaaaaggttgagccaaaaagcccccagtgtttgtattttgaagccaagcttcacttctaaagtgcctacagagacttgtaaatgaaaatgcagctctgcacgagtttgaaaccgtcatacctccttctattaggaatggcatatactgaggtggtcgtaagtcttaacttctaaaattttaaataaaagactttgcacattgaaaaaaaaaaaaa

SEQ ID NO: 71 - DNA, 1588 bp, Homo sapiens
atcttgctttcctttaatccggcagtgaccgtgtgtcagaacaatcttgaatcatgaagctactaaccagagccggctctttctcgagattttattccctcaaagttgcccccaaagttaaagccacagctgcgcctgcaggagcaccgccacaacctcaggaccttgagtttaccaagttaccaaatggcttggtgattgcttctttggaaaactattctcctgtatcaagaattggtttgttcattaaagcaggcagtagatatgaggacttcagcaatttaggaaccacccatttgctgcgtcttacatccagtctgacgacaaaaggagcttcatctttcaagataacccgtggaattgaagcagttggtggcaaattaagtgtgaccgcaacaagggaaaacatggcttatactgtggaatgcctgcggggtgatgttgatattctaatggagttcctgctcaatgtcaccacagcaccagaatttcgtcgttgggaagtagctgaccttcagcctcagctaaagattgacaaagctgtggcctttcagaatccgcagactcatgtcattgaaaatttgcatgcagcagcttaccagaatgccttggctaatcccttgtattgtcctgactataggattggaaaagtgacatcagaggagttacattacttcgttcagaaccatttcacaagtgcaagaatggctttgattggacttggtgtgagtcatcctgttctaaagcaagttgctgaacagtttctcaacatgaggggtgggcttggtttatctggtgcaaaggccaactaccgtggaggtgaaatccgagaacagaatggagacagtcttgtccatgctgcttttgtagcagaaagtgctgtcgcgggaagtgcagaggcaaatgcatttagtgttcttcagcatgtcctcggtgctgggccacatgtcaagaggggcagcaacaccaccagccatctgcaccaggctgttgccaaggcaactcagcagccatttgatgtttctgcatttaatgccagttactcagattctggactctttgggatttatactatctcccaggccacagctgctggagatgttatcaaggctgcctataatcaagtaaaaagaatagctcaaggaaacctttccaacacagatgtccaagctgccaagaacaagctgaaagctggatacctaatgtcagtggagtcttctgagtgtttcctggaagaagtcgggtcccaggctctagttgctggttcttacatgccaccatccacagtccttcagcagattgattcagtggctaatgctgatatcataaatgcggcaaagaagtttgtttctggccagaagtcaatggcagcaagtggaaatttgggacatacaccttttgttgatgagttgtaatactgatgcacacattacaggagagagctgaacgttctctcacccagagcagcaaacacatgaaagtcagaagtctctaatatatcatttgtcttttttccagtgaggtaaaataaggcataaatgcaggtaattattcccagctgacctaaagtcaataaaacattctgttt

SEQ ID NO: 72 - DNA, 1331 bp, Homo sapiens
gaggggagggggttggccgggactcgtttgcgatgttccgttatctggatgcggcggagggatctggcggagggaggtgtttatgaggcgctgggggcggaggaggcgaattagtccgagtggagagagcgagctgagtggttgtgtggtcgcgtctcggaaaccggtagcgcttgcagcatggctgaccaactgactgaagagcagattgcagaattcaaagaagctttttcactatttgacaaagatggtgatggaactataacaacaaaggaattgggaactgtaatgagatctcttgggcagaatcccacagaagcagagttacaggacatgattaatgaagtagatgctgatggtaatggcacaattgacttccctgaatttctgacaatgatggcaagaaaaatgaaagacacagacagtgaagaagaaattagagaagcattccgtgtgtttgataaggatggcaatggctatattagtgctgcagaacttcgccatgtgatgacaaaccttggagagaagttaacagatgaagaagttgatgaaatgatcagggaagcagatattgatggtgatggtcaagtaaactatgaagagtttgtacaaatgatgacagcaaagtgaagaccttgtacagaatgtgttaaatttcttgtacaaaattgtttatttgccttttctttgtttgtaacttatctgtaaaaggtttctccctactgtcaaaaaaatatgcatgtatagtaattaggacttcattcctccatgttttcttcccttatcttactgtcattgtcctaaaaccttattttagaaaattgatcaagtaacatgttgcatgtggcttactctggatatatctaagcccttctgcacatctaaacttagatggagttggtcaaatgagggaacatctgggttatgccttttttaaagtagttttctttaggaactgtcagcatgttgttgttgaagtgtggagttgtaactctgcgtggactatggacagtcaacaatatgtacttaaaagttgcactattgcaaaacgggtgtattatccaggtactcgtacactatttttttgtactgctggtcctgtaccagaaacattttcttttattgttacttgctttttaaactttgtttagccacttaaaatctgcttatggcacaatttgcctcaaaatccattccaagttgtatatttgttttccaataaaaaaattacaatttacccaatggttgctctgcatctgagtcatttaactgttgaagtctaataattttgaaaataaaatatggcattggtttctgcttggtaaaaaaaa

SEQ ID NO: 73 - DNA, 971 bp, Homo sapiens
gcactgtgcgtcatgtaccagcgccggaagttggtctcgacacctggactagccgggttgtatttggaaacgcggagtgagtttttccgtgctgtgtaggggctaacaatggacacccagaaggacgttcaacctccaaagcagcaaccaatgatatatatctgtggagagtgtcacacagaaaatgaaataaaatctagggatccaatcagatgcagagaatgtggatacagaataatgtacaagaaaaggactaaaagattggtcgtttttgatgctcgatgaatgctgggaattcagaggaatgtcttcacttatacttggatttgctctcttcccatttctgattgttgtatagctttcgattttgcttacagtagttcccccttatcttcgggagatacattccaaggcccccagtgaactcctgaaacctcaaacagtaccaaacctttatacactgttttttccatatatatatacctatgataaagtataatgtataaattaagcatagcaagagattaataataatgtaatagaacaatgataacatactataataaaagttatgtgaatgtggttggtctctcttgctttcaaaatatcttcttgtacagtactcacctattttagaatgtggttgactacaggtaaccaaaaccacagaaagggaaactttggatgaggggggcactactgtacttaggaatacaactatatacatatgattttatttttaagaccatattatatttgggtatctactaatattttgtataaagcaattttttgttccattacgtgactttttgttttattgtatatgtaatttaacacacaataaagggtaaagttgcttccccaaaccacacttttaatcaaaacctagaatcatctgcagtccttgttaaaaatgcaggtttctagaaccctctgaagttctgattaaataaatttattgcaaacca

SEQ ID NO: 74 - DNA, 18687 bp, Homo sapiens
gatcctcctgcctcagtctcctaaaaggctgggattacaggtatgaacccccaggcctgcctacttcgtgtgttttatctggcaatgctgagaggacagctagatgtcgaggacagccagatttcgaggacagccagacgtcaagggggaaagcaacttgctggctcacacagccagccaggtctattgtggagccctagtcgcaaacacatacaccccagctttcaagtttttaaatatttatttagttagttatttgttttttgagacagtcttgctctgtcacccaggctggagtgcagtggtgcaatttcagctcactgcaacctctgcctcccagattcaagtgattgctgcctcagcctccctagtagctgggactacatgtgcgcgcctgcactgcccagctaatttttgtacttttagcggaaacggggtttccccatattggccagagtggtctcaaactcctgacctcaagtgatccacccacctcggcctcccaaagtgctgggattacaggcgtgagaccgtgcccggctagctttttttttttttttttttttaattaggttgaatgcttgttgagctgggtgcctcacatccgttgtctgttatacaaagaactactgattatgggtctctacgagacaaaaatggcagaacacctacagggcacaggggagatgggagcagggattaggcttcagaatacttctccaaatgagggtgatcagggcccgacatttcactgctctaaggctcagtatcttaactatcaaatgggtacaatggggcacaaagggttactttgaggaataacggaggtgagcaatttccaaaaagttcattcctttaaaaactgcggagcacattttgtgtacatgctgtttttattgttactcatgaagacgcgggggaagctttggttgtgtaaagctgagaactgcacccaagcttccccgttcaccccacctaccaggggatttgggctaaattcctggcctggcccacaaagaagaccaagcggtcagtcccattccgtcccatcctgatttacaggaactcacaccagcgatcaatcttccttaatttgtaactgggcagtgtcccgggccagccaatagctaagactgccccccccgcaccccaccctccctgaccctgggggactctctactcagtctgcactggagctgcctggtgaccagaagtttggagtaggtttggtgctgggcaggggtggggagtagggtggaaagcatggagtgaagaggtctagggagggggtctcctcacccccgccttcctgcccgccttgatctcgggggtctctataggcttgcttccacctgggacttctgcctcctcctaccccagcccctcccgcctcaggcctgttgtgctcagccccccaggacctcccctcccccacgcctctggcctcattgtttggttaaagcaggaccccctccccctcccaccacctcccctccgactgaacagatggacagagacccgggcccacggggagaggaagggccagccggtgccgggaaagggaagcggtttggggaaaacaaaacagagggaggagccagggagaaggtggccccaggagggaggaggagggaattcgctaagagggactggggcctgagacggaatgggggcgggccccgggcggggtgggggcccctgggctcccagggtgggagctggctccgaggctggctggctccgcaggtccgctgacgtcgccgcccagatggcctccaggctgaccctgctgaccctcctgctgctgctgctggctggggtatgtggtcccttgtgggatgggggacgggggtggagacgggaggcgggatggtgcggggtgcgggcggtggctgaggattaacccttcaggctccggggaatgaggagagctcctcttgggatcattgagtgtgatccttgcacacgcactcgtagatggtggaaagagctcaggactcagacagatgcaagttctaatcttggcgggccatgtaggagctgcgcacgtggtcctgaggacgtcacttttctcagcctactcctcctgatttataaaatgttgaataaacagatcccaccctgtccaccaagcaggcttggcaggaggatcacatagggcaaggagatgggaagcattttgtgatatcaaagcaagagtttgttgtagaaaagagctgtggggaggagaggtttggctgctgtgacacagacggtggtcccaggctagggcctgtgaagacagggaagaatggtagcaaacatacgtgggtctgggtgggggtcaatagcagtgagaggtcccagtggtctgggaggtcatggactagaaataaggctaagaagtgcaggatcccaggagaaaggtaggtaggggtgtgtgtatgtgcgtgtgtgtgtgtgtgtgcacgcgcaagttggtatcacatactcagcagatgatgtagttatgagcagggagtttggaatcagagaccttgttttgacccctagctgtttctcccatttctgtgagaccctggactagttaatatgtgtcggtatgagtttctctacctgtgaaatggagctaatgcgtggtttctcagtggaggatctgttgacattggggctggataattcattgctgaggggctaccctgtgcattttaggatgttttgcagtatatctctagcctctacgcactagattccggtagcatcttccctgctgtctccctgtgacaaccaaaaatgtctctggacattgccaaatatcccctgggagtcaaaattgtttcagagccgctggcctatactacttctctctccctcccttcctgcctcccttctttcctccttccctttcttcgttcccttcctaatgtgtactgtttttccacacccactaatgggttgcaacccacagtttaaaaatttactgttctaaatcaaggagcacaagttcaaatgtctgtactagccaagcaagtgagtcaaacaggtagatatttacaggaaattgcaagaacatagataaatgacaacactcagcttcactgtttggaaaacaacttcctacagggcagtaattggtcagagattacagagtccctgactatccctcatcttctgcagagcacattcctgtgcacccccaccctcaccctgtattgccccttctctgaggaattagtggtggtggttctaagacagattgctcatctgctgcactgtcagaaattactctcttgtacaggacattttccacatccacaccttctcttcctgctttgagtattttagaggactgtgcctcgtagtaagaaaaaaatgaaactcagtttcttgaaccacaggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggtaagaccctgcttgaattctctccaggtcatttgttggacactcccataagagtcaccaatccagacacttacaaagccatgcctctgggaagaagctgtaaaaatgggctattatatattgggggtggggtagagggatgtatcttttcattcttgaacattccatcatttcacagtgatgtaataggcacgattgcttgtaaaactctgtgactatacaagaacatataaaataaggtcgcagccactaaccatgtttcatggcaaggagaggtgataagaaagatgaaattaggcgcagtggctcacgcctgtaatcccagcactttgggaggccaaggcgggtggatcacctgaggtcaggagttcaagaccagcctggccaacatggtgaaaccctgtctctactaaaaatacaaaaattatcagggagtggtggtgcatgcctgtaatcccagctacttgggcagctgaggcaggagaatcgcttgaacccaggaggtggaggttgcagtgagccgagaccgcaccattgcactacagtctgggtgacagagcgagactctgtctcaaaaaaaaaaaaaattatcagagatagacctagagtagatgtggttagtactgccttctagctctgtgaccttgggcagatcactttaacctctctgagccttgagtcctcttgtgtaaaatagtgatgatgctatctacctcaaaagattaagaagcagaaagccaggccgggtgcggtggttcacacctgtaatcccagcattttaggaggccgaggagggcagatcacgaggtcaggagttcgagaccagcctgactaacatggtgaaaccccgtctctactaaaaataaaaagaaattagctaggcatggtggtgcacacctgtaaccccagctactcaggaggctgaggcaggagaatcacttgaacacgggaggcagaggttgcagtgagccgaaatcatgccactgcactccagcctgggaagactgagcaagactctgtctcaaaaaaaaaaaaaagaagcagcctagtgtctgacttagtgggaggtcaaaaaaatgtaaatcctctgccatcttgagggattactgtcaagtcccatttggtaattaccctaggaatggcacaaacaaattactacaagcagtggggacagagctattactccccagagagaattctaaaaaggctacagaatctttcttggctgggcacggtgactcacacctataatcctggcactttgggaggccaaggcaggagttcaagaccagcctggccaacatgttgaaaccccatctctactaaaaatgcaaaaattagccaggcatagtgatgcatgcttattgtcccagctacttgggaggcagaggtgggaggattgcttgaacctggagattcaagtgagctgagattgcaccactgcattccagcctgggccaacaaagcaagactctgtctcaaaaaaaaaaaaaaaaaaaaaagagagagattgagagaacattccagctcagatgatctgtgatcccctccaaagcagggaataccctccattccagcctggtccccaaccctcattcccaaggaaggcccccgactcatcctgcaagtatctttcatctctgccctttgttgcaggggctggggagaacaccaaaacaaacctggagagcatcctctcttaccccaaggacttcacctgtgtccaccaggccctgaagggcttcacgaccaaaggtgtcacctcagtctctcagatcttccacagcccaggtgagtgcccaggaatgggcagtgtctgcagaggagggtcctgagaggactctgaagggggacccagcgctggggaaagaaaggacagagggaatgttggagctacagtatcagggatggactgcagagcaggtgaagaccttggcaggagcattaggtcactccaggaactagactgttcttctaatgagaccttagacaagtctctggcattcatcaactgctttagaataaaaataaccgggcaggtacagtaaaatagtgatgatgctatctacctcaaaagattaagaagcagaaagccaggctgggcgtggtggctcacacctgtaatcccagcactttgggaggccgaggcaggtggatcacgaggtcaggggttcgagaccagcctgaccaacatggtgaaaccctgtctctactaaaaatacaaaaattagctgggcatggtggcgggcacctgtaatcccagctattcaggaggctgaggcaggagaattgcttgaacctgggaggcggaggttgcagtgagccgagatgacgccactgcactccagcctgggcgacagagcaagactccgtctcaaaaaaaaaaacaaaaacaaaacaaaaacaaaaaaaaaaaacaaagaaggagaaagccgggccgggcatggtggttctcatctgtaagttcaaggagttgaaggtatgctaggactttgggaggccaaggccttcaagaccagcctgggcagcatggcgaaacctgtctccattaaaaaaaaaaaagttgggggtacggctgggcatggtggctcacacctgtaatcccagcacttttgggaggctgaggtgggtggaacacctgaggtcaggagttcaagaccagcctggccaacatggcaaaaccctgtctctattaaaaacacaaaaattagcctggcatggtggcaggcgcctataatcccaactactcaggaggctgaggcaggagaatcgcttgaacccaagagagtgaaggttgcagtgagctgagatcatgccacttcactccagcctgagtgaaacagcaaaactctgtctcaaaaaaaaaaaaaggaagaaagaaaaaaggccaggcgcggtgactcacgcctgtaatcgcaacactttggcaggccgaggcaggcgattcacaaggtcaggagttcgagaccagtctggctaactaacatagtgaaactccgtctctactgaaaatacaagaaattaccctggcatggtggtgtgcacctgtaatcccagctactcaggaggctgaggcaggagaatcgcttgaacctgggaggcagaggctgcagtgagccgagatcgcgccactgcactccagcctggatgacagagcaagactctgtctcaaaaaaaaaaggccgggcgcggtggctcatgcctgtaatcccagcactttgggaggctgaggcgggcggaccacgaggtcagaagatcaagaccatcctggctaacaagatgaaaccctgtctctgccaaaaaaatacaaaacttagccgggcatggtggcaggcgcctgtggtcccaactacttgggaggctgaggcaggagaatggcatgaacccgggaggcggagcttgcagtgagccgagattgcgccactgcactccagcctgggcaacagagcgagacaccatctcaaaaaaaaaaaaaaaaaatggggcgggcgggccaggcgcggtgtctcacacctgtaatcccagcactttgggaggctgaggtgggcggatcacttgaggtcaggagttcaagaccagcctggccaacaccaggaaaccctgtctctgttaaaaatacaaaaattagccaggtgtggtggcacgcgcctgtagtcccagcaggagaataacttcaacctgggagacagaggttgcagtcagctgagatcgcaccactgcattccagcctgggtgacagaccgagactctgtctcaaaaaaaaaaaaaaagaagaatacccatatgcattcattaatatatagggctagagggctagagagctatagacataaaatagacaaaaaatttttttgctcatttttgggtcaaaggagtcttgggactctaattcttttaatttttgtgttatgtgaatttgttatcatttacatgtattatgttattaagtaggtaataatgataatactaataataaacttacaaaacgatccaatgtagttgttttcagactttgttcctcggagccctaggactttgcaaagctgtttctggagtcatggtgggagtggggtgtggggagcctgagcagtggggaagggttcaccacttcctgtgagagggaaacggaccagctgggctctgagctccccacccagcctcagccagggtccattttatattgtgggcttcagacatgcctttgtttgaaagcagttctgctgctttaaaatgtttgataaccattgaactaatctacccacctccctttttaaaaaaaaaaggaaaacttttttttaagctgttttttttgttttttgtttttttttaaactgtaaaaacaatacttaggtctggctcagtgtgcctgtaattccaacactgagaggctgaggtaggaggattgcttgaggctagaatttcgagactagcctctgggcaacatagacagacctcatctctacaaaaaattttaaaaattatccgggtgtggtacgtgtctgtagttccagctatgaaggactgagacgggaggattgcttgagccaagaattcgaagttagagtgagctatgtttatgcctctgtactctaggttgggtaacagagtaggaccctgtctctaaaaaaaaaaaaatttaatttaaattttaaaaactcacctatcaccccatcatctgaaaaacaccctctttcagatgtgggaaaaacatggctcagaggtgttcaagaaatgttgcatttatttattacttacaaagctgtaagtcagctttatccgctgtcttgctaggttggtgaaaaatacgtaacagcattcaatattagggcttctatccccatctagctgcacactggagctgtgccaagcaaggttcttggtcttatgttgtacttaggcatcagcaaagcctgggaaaatatcttttttttttttttgagacagaatctcgctctgtcgcccaggctggaatgcagtggcaccatctcagctcactgcaacctccgtctcccagattcaagtgattctcctgcctcagcctcccgaatagctgggattacaggctggtcttgaactcctggcctccagtgatccacccaccttggcctcccaaagtgctgcgattacaggtgtgagccaccatgcccagctggggaaaatatctttaatctacttagtcctagaaaaattatctttcaagtgctttttttttttttttttcagtggactgaaggcttttgcagagcaaaagccaggaagttttgtcacaaacttccaccaaggccaggaagcagagtctattccgtgccaaaattaagggaagaaaagagggaaaatctaggcaaatggaatttaatatctcaaaaagattatcttgccgagccatagtcacatagcaagctgaggcagcggaggcttggggctacttctcttgttcttggttctgggtttaccttctttgggccttatttgccacatctgtaagaggagtgggctggaccgcgctccaccatgccgtattcactaagtgagcagatagaaccatagaaagcatgctcactctcaaatcgtgctcatggaaagaacgacgtgttcaggactcatgcctccctttctcaacatacccccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtggtaagggtgcccttagccagttagtcttcccattctgggtccttcttcccctcctggcttcaaagcccacttaaccccaagttctacaatcggatctcaatgtccctgcactactctttgctaacaaggcttttagctcctcttcatccttttcctacctgcattagagcaaccctcccacctcttccctctagccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtaagttcttaacctttccttctcctgtttgaaacctacttgagtctcctgactttttttctgctgtagtcccatcattttggggtacatgcttacaaattcatcacttctactccttccatctgtatttccaccctatcttttctccttctcctttctctagccttggccaaggcagacattgtatattttaggctggaaacaggattctcaagtttcttcatgcatctctactatattgaatggcaaaatgtgagtcgtgttcctattctacacatctgtttctttttccagtcaactcatcagaagactctggtggcttagcaagtcctgtgtgtatgtgggtacctgtgtacagcatacatatacatgtgtagacgtgtataagtatatccttgtatatgcatatatgtggtaagtgcatgtgtgcacatgagttaaatggcttaagtgcattagcagaaattgaacaatttagtttgatgagtagggagggagagaagacagaatatggagcccagaggatcccaactggcaagctatctcatccccagttaccagatatttccacctttttaggatggcaaaacacagacctatgagacagaaaacagtaagagacaatgtctcaatacctggctgagaaacaaaggctctgttcttggaacatgctaggcttggttccacattgggagctttccttagcttttgtttctgcctgtatccctcttcactcagatcctcacatagcttgttccttcttgtcatttgggtgtcagctcagaggtcaccttggtggagaagtcttctccccattaaagcagcctcctcctcaccctgtaatatcactcctgttttattatattcattacactcatcaccatctgaaattatgtcatttatttgcttatttgcctaggacattgtctgtctccctaagatactgtctctctacttgaatacaggaacctgtcttgtttccttctgtatctgtagtgcctagaacaatgcctcctatcataggtgctcattcagtgtttgctgaataaatgaatgaatgaaatagagggtgccaaagagagctactcagtaaaagtggctggaagacttcatagaggagggtttttttcgagacagggtctcactctgtcactcaagctgcagtgcattgacatgatctcagctcactgcaatctctgcctcccgagttcaagcagttctcctgcctcagcctcccaagtagctgggactacaggagcccgccaccacacctggctaatttttgtatttttagtagagacggggtttcaccatgttagccaagctggtctcgaactcctgacctcaagtgatccacaccctcagcctcccaaagggctgggattacaggcatgagccacagcgcccagctgaggaggttatttttgaccattctttgctcagttatttatccactcatggacttatccacccatctatccatccctatctaatgtatttgaccattcatgactcatttagcagaaccaggacagcctgtataaggcacagaagagtgaatatatgtgacaggtccagagcattgttcaaattcaagatacgttgggggaggtataaatatgagatttttttcaaatcaattggaatggctatttaatcaaagccatagatgttaggtggaaagtaggaattaagaacttaacaagttaggtttaaatgtaactattcgaattataagaacaagtatttcctctctaaaaatgtttttaaagtttcacacaaaactctaaaccaaaaatcattgtaaaaatcccttggagtagactgttgaaaccattgaggtgaggtgataggttaaggtgagcattctggacatataatatcaataataaatattcaaatacaaagcaggtataatatgaagagtaatttctatctccccaagccaatatgagttttgtttgtttgtttgtttttcttttgagataaagtctcattctgttgcccaggccggagtgcagtggcacaatcccagctctctgtaacctctgcctcccaggttcaagcgattctcctgccttagcctccctagtagctgggattataggtgcctgccaccactcccagctaatttttgtatttttagtagagatggggtttcaccatgttggccaggctggtcatgaactcctgacctcaagtgctcgtccgtcttcggcctcctaaagtgctggcattacaggcgtgagccactgcacccagcctgagttttttttttttttttaagccaaaggaggaggagtagttttgtgcacaaaagccactatttagagttccttaggttttaaacagataattaatttaatgtactaacaaatgcaaaattaaaattttacttatgccttttaattttaagctgaacttgctacattaagagtagggtgatttggctgggcgcagtggctcatgcctataatcccagcactttaggaggctgaggcaggtggatcacttgaagctaagagtttgagaccagcctggccaacatggtggaattctgtctctactaaaaatacaaaaattagccaggcatggtggcacatgtctgtaatcccagctactcaggatgctaaggcacagaatcacttgaacctgggaggcagaggttacagtgagccaaaatcgcgccactgcactccaacctgggcaacacagcaagactctgtctcaaaacacaaaaaaagaataggctgggtacagtggctcacgcctgtaatcccagcactttgggaggccgaggcaggtgaatcacctgaggtcaggagttcgagaccagcttggccaacattgtgaaaccccatctccattaaaaatacaaaaaataagccgggtgtgatggtgcacgcctgtaatcccagatacttgtgaggctgggacagaagaatcgcttgaacccgggaggcgaaggttgcagtgagccgagatcatgccactgcattccagcctgagcgacagagtgagaccttgtctcagaaaaaaaaaaatagggtgatttttatataaaaacactctcatttgttcttgaattaattgccatattgaacagattaaactcctttaaaccattttttctatctacaaacataattagtattcccataagcattttaagctccctttaaaaatttaatatgctcgattttcttttttcttttatttatttatgtagttttcccagacagggtcttactctatcacccagtctggagtacagtgacacaatcctcacttactgtaacctcaagctccttgggatgaagcagtcctcccaccctagcctcttaagtagctgagattccaggcatgagccacctcacccagctgattttctttttaaacacatctaatacctgacaggtcgtaattacgactgtgcttggccatatcatcctaacacctaaagctcacttgtaaactgaaaattaaatttgaagggtgaattaacttctagccaacattccaatatcattctcacatttaatgaaattatcctacaactttgcttaaaagcataaaactagcatgtcagagtctcttaaaaattacaatcactattttaaataacaaacatacaggttaccctgaacttaacacctggttttaacatgatgaagttggtttcttttgtcattcttatactaaattctaaatcttcctggcacttaaggatatttacccaaggaagggggtgagatttaccggctcaggtacaccgaggttactggcttgaaaattcaagaccacagcatggtaaatttttcttcagagattcaaggttcatacataggacttgaagggcacatgctactcaaatgagtatatccaacttgagtgctacatcaacagggaccccatgtccactcttttcagtcgaaattggcttactatctacttcttctgggctattcaggttccgacaattgattgctttcagttaatttctttattgcccagttgaattctgcattcttctagattctttttgttccttcagcctcaatcataataccatttaatgacagtctcatatcctattagactagggtttctcagccttggcactattgacatattgggtcagataattctttgtcctgtgcattgtagaatttagcatcttccttagcttctactcactagaagccagtagcactgtgtgcacatcctccttgccccctgccagctatgacaaccaaaatgtctccagacattaccaaatgtccactgagaggcaaattcactccgagttgacaacccctgtattaaacccaggggccctgcacttcttacatacttttgtctggtcaccagttgaaagtccttcttcttttttttttctctttttgagatggagtctcgctctgtcaccaggctggagtgcagtggcatgatctcggctcactgcagcctccacctcccgggttcaagcaattctcctgcctcagcctcctgagtagctgggattacaggcacgtgccaccacccagctaatttttgtatttttagtagaggtggggtttcatcatggtggccaggatggtctcaatctcttgacctcgtgatccgcccacctcggcctcccaaagtgctgggattacaggtgtgagccaccgtgcatgaccaaaagtccttctaatagcaaagtggaaaggtaggctgtgaacaatcttgaatgccatactaagatttgtacttattctcataggcagtgaagagccatcaatagtttttacagaggctgacctgataactctggcggttcaatgtgcatgctatatactatatgcacaacacattaggaaggaagggaacagcgctcagagaaatcaaatcacttgcccatattacatagctcgtcagtggtggagtcagggtataatcagaaactcatgctccttcctctacctctccccagcacagagttggatcagcactgttcacccagctggtatccccatttcatggtaaagcctggaagcttaggtctgactgatgcttgttgaaatacagactgtgggagcagactgcaggacagcattgtgacagagggtggggccaggagagagatgcggtaggaagactgttaagatgcatctcttattttctaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattgggtgagctctggcagcttagggttactcccaggccatcagaggagaaagggggatccctaagatgtagttagcattctctagagtattttttacatccataatctcagtttgtcctgcaaccctgcaagttagaggggtaggtgctattatcccattggatcattgtggaaatggaggctcagaagctttatgtgacttacccagagattccatgatttacccttgagctagtcagaggcaaaccagaacaccgacccaggtctccagctctcctgagttttttctatggttccttgtgacataggcagtggaacagtgggacaggtaaccgaggtgaattatggatgctccttccccagacacatttcaaaacagtcagccaccccgtggaatatgcaagaagctatctggaagtgcagatctggaggcttctgagtttacgagaggcaacagagactccattttcttttttttttttttttttgagacggagtcttgctctgtcacccaggacaggctggagtgcagtggtgcgatctctgctcactgcaagatccgcctcccagattcacgccatgccattctctcgcctcagcctcccgagtagctgggactacaggtgcccgccaccacgcctggctaattttgtttttgtatttttagtagagacggggtttcactgtgttaaccaggatggtcacaatctccggaccttgtgatccacctgcctcggcctcccaaagtgctgggattacaggcatgagtcaccgcgcccagcccagagactccattttctaacctttgtttttttgtttgtttgtttttgagacggagcctcactctgtcacccaggctggagcgcagtggcatgatcttgggtcactgcaacctccgcctcctgggttcaagtgattctcctacctcagcctcccaagtagctgggattacaggcacctaccaccacgcccagctaatttttttgtatttttagtagagatggggtttcaccatgttggtgaagctggtcttgaactcctgggctcaagtgatccacccacctcggcttcccaaagtgctgggattacaggtgtgagccactgtgcccagcctcattttctaacctttgatctcatgtccagccctgtcacttcatttcttgttagagaatttgaccgcctaacacatgattccatttccttgtatatgtcatctgtataggaagaaaacctcttttttattttcattcaacacttctggccaccaaacaggtgtgggagtgttttctaagcaattctccagttatctggggacaccaactgggtgtcctacaattgaatccaattctaacactctacctggaaataacatcagatcccgtgcagtttaagtgctccgccccagcaaatttccccccgtttccaatgtcagttacaagactgggttgtcacctgtgcatcagaccaaccagctataatttggagattcccacaactccctgctagggtttcatcatttgctagaatagctgacaaaactcaggagacacttacatttactggtttattatgtaaaggattaagtaaaggatacagatgaacagccagttgaagagatacatagggcaaaatctggaagggtcctgggcccagcagcatctatcctgtggagttctgtttgctaccttcctgacacatgtatgtgttcaccaaccagaaactctcagaaccctgtactttaggagaagtcatcatgtaggcatgattgattattacccaatctccagctcctctcttctccccagaggatggagagaggggctggaaagttccaagcttctaatcacacttggtctttctggtgaccagccccaatccaggagccagcaggagtagcctcattagcacaaaaggcattcctatgacccaggaaattccaagggatctagtagctctgtgtcaggaactgggatcggagaccaaatattagatggtctaatatttggagctaaagatgctcctatagcatattttcccattatttcacagcatcatggcattgcatataacctcattttttcatatatgacctatctctgatctattacctcacttccaaggtcctgggagataaggaggcaatgacatcatttttcagggttgttttagggatgaggggcaaggcctctcaccttgagagagagtggcagtgttgggcccactaatagaggatcccacgaactgccagagggtacagtatgtaatctggcaaacaagggaagaggaagagagcactgggactcaggatgaacccagagaattcaggacaaaggtctccatcagctgagggtatcatgctggcttctgactctgtttttctctggttttgccctagaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttgcccctgccacctcctgcctcaggtgtccgctatccaccaaaagggctccctgagggtctgggcaagggacctgcttctattagcccttctccatggccctgccatgctctccaaaccactttttgcagctttctctagttcaagttcaccagactctataaataaaacctgacagaccatgactttctctgctttgcctttgtttttttattttttattttttattttttttaggcagggtctcaatctgtcacccaggctagagtacagtggtgagatcatggttcactgcagccttaacctcctgagctcaaacaatcctcccatctcagcctcctatgtaggtgggaccacatgggcttgccaccatgcccagctaatttttaaattttttgtaaagacagggtcttactatgttgcctaggctggtctccaactcctgggctcaagcaatcatcctaacacagcctcccgaagtgctgggattgtaggcacaccatacctctcctttgcctttgaatatcag

SEQ ID NO: 75 - DNA, 3764 bp, Homo sapiens
aacgggggcgcgcggtcgggggcaggttgggccggaagaggggtccgggccggcactgaggcgggaagggggtcggttgagatgggacggccggagtctgcgggcggagggagtcgcgggccttttgagggaggaggcagagcgcgccgggccggtggcatcttccttactttgtccatcctccggactcgcgatcttccttccggagccatgtcagaaggagtggacttgattgatatatatgctgacgaggagttcaaccaggacccagagttcaacaatacagatcagattgacctgtatgatgatgtgctgacagccacctcacagccctcagatgacagaagcagcagcactgaaccacctcctcctgttcgccaggagccatctcccaagcccaacaacaagacccctgcaattctgtatacctacagtggcctgcgtaatagacgagctgccgtttatgtgggcagcttctcctggtggaccacagaccagcagctgatccaggttattcgctctataggagtctatgatgtggtggagttgaaatttgcagagaatcgagcaaatggccagtccaaagggtatgctgaggtggtggtagcctctgaaaactctgtccacaaattgttggaactcctaccagggaaagttcttaatggagaaaaagtggacgtgaggccggccacccggcagaacctgtcacagtttgaggcacaggctcggaaacgtgagtgtgtccgagtcccaagagggggaatacctccacgggcccattcccgagattctagtgattctgctgatggacgggccacaccctctgagaaccttgtaccctcatctgctcgtgtggataagccccccagtgtgctgccctacttcaatcgtcctccttcggcccttcccctgatgggtctgcccccaccaccaattccacccccaccacctctctcctcaagctttggggtccctcctcctcctcctggtatccactaccagcatctcatgcccccacctcctcgattacctcctcatcttgctgtacctccccctggggccatcccacctgcccttcacctcaatccagccttcttccccccaccaaacgctacagtggggcctccaccagatacttacatgaaggcctctgccccctataaccaccatggcagccgagattcgggccctccaccctctacagtgagtgaagccgaatttgaagatatcatgaagcgaaacagagcaatttccagcagtgccatttccaaagcagtatctggagccagtgcaggggattacagtgacgcaattgagacgctgctcacagccattgcggttatcaaacagtcccgggttgccaatgatgagcgttgccgtgtcctcatctcctctcttaaggactgtcttcatggcatcgaagccaagtcctacagtgtgggtgccagtgggagctcttccaggaaaagacatcgctcccgggaaaggtcacctagccggtcccgggagagcagcaggaggcaccgggatctgcttcataatgaagatcggcatgatgattatttccaagaaaggaaccgggagcatgagagacaccgggatagagaacgggaccggcaccactgagaaaggagtctggttggaagcaaatgtttttttaatggacttgcatctcctcaccttgatcaggactaaaggacggaggccgccccacccccttccctttcctccaaacccctaactccctccagacacccagggaataccctctgccccacaggattgaagactgcttggcagtcctcccaatcccacacctcctgtttgccaggggaaagaacctaaagacttcgtgtgattgggaggggtggcagacaggaagaaaacatgtccaggcccctggtctccatagagaatggtgctttgtccaagaaaacgtatgagtttctgattctccgggagccgttcaatggtgaggttgatgggaagacttccttcccaaagaaaatagatcctccatgcaggatctaggagagtgactgggtgtgccaaaatatgcccagggtcctgccctcagcactagatttaatggggccaagagggtccaaaccccttgctaacataccacttctttgtttaactcctttacctttccagccctttgaggagggaccatgagaacagaaattaccttatgaaaagctacttctgttcctgctttccctctcacgtattgacggtttatttctttgacctcccagagggctgaactctttcaactctgcgctgcccagccttctcagtggacttgcccctcctaagcagagaaggcctatgaggttgcttgctgctgggaagcctggcagagccaattaccaccctctgctgcttagtgcttgggtacctcttgcaataaccagctcttagttgttccctttccctggggcttttccatttaacacatggagcccttcccccagaaggctacttccttgttttagaggaaggtactgcccattgggagatggggacattgggacctcagcaatgaagaacccttgtgaagtaaccaggaggaatggggaaagaagcaagttgggcaggatatggcctacttccataggcttttcttttttcaggtttgatgtaagcatgggcttacatcccccaggtacatacttttacttattgtgggataacctggcactagtaggcaggtaaagtcacaaatttggtgtcttttcaccttttgactgttgacttaatagctcctctcactctgcctggagatacttcctgcctcagatgaggagccagaagaaacagagcccgacttgaatgaactcagctcagagttctaaggaccagcattctgggggccattttctctacaggcaaatggaattgcttttccataacatccaaattgtaatgtggttgctgctgaaggaggaggcagcagcgaggtcctgcggtacccatggggtgatgctacttctgcatgcatctacagggcatctgacacctaacatgagacgtggcatgtgagatgagacttggcatgtgagacatagggtcactagagacccttctgggtcagaggagagagactgaattggactaaacccgtcctctgttcccagcacgtttctcatatagccctcagtcactgagggagtcccccgcaggattggagaggcacattcccttgggacagaggctacaggttggagctttttttcccctgtcccccaaccccatccccacctccacttcagaacatggcaccccacccaactggccaagtgttaagtgatgtgcttattgagagcaactccgggtgtcttttaaaatgtagagaaaaggtgacagtttaaggaaaaatatatatagaataccagaaatgccgtttacccggagaatttttttctccccatttgttttgtttttactcaatgacaccatttttagttttatttcctgatagcaaaaggaaaaaaaacacccatccctcaaaaaggccaaggtcccgtccccctgttgtcggtgatttgtttgtctttctgataggttgaaaattgtgtaataaacttgatgacgctgtcaatcttttatactgcattgtatttttttccttttgtaacaaaatatttttaaataataaatggggtgtgagctgttttatggaaaaaaaaaaaaaaaaaaaaaaaaaaaaaaaaaaaaaaaaaaaaaaaaaaaaaaaaaaaaaaaaaaaaaaaaaaaaa

SEQ ID NO: 76 - DNA, 3130 bp, Mus musculus
gctggctaaaggagcagtttccccgaccctacacgcctcccccaccgcacctcctccgccctgttcctgggcccctcccctagagccctagcttgacctaagctgcttgctggtggagagcacaccatggcctcaccgttgacccgctttctgtcgctgaacctgctgctgctgggtgagtcgattatcctggggagtggagaagctaagccacaggcacccgaactccgaatctttccaaagaaaatggacgccgaacttggtcagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccatgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccgaaagcgtgtttgcaaatgtcccaggccgctagtcagacaggaaggcaagcccagaccttcagagaaaattgtgtaaaatggcaccgccaggaagctacaactactacatgacttcagagatctcttcttgcaagaggccaggccctcctttttcaagtttcctgctgtcttatgtattgccctctgtattgttttagtaggggtgtgatggggacagttcctttttctttatgaattctctttgacacaaagcatacttgtatgcatacaatgggagtaatgagcagactgtaacaccagagctagttccagtttcggggtccatgtcgctggtggcctcagcacccacttgatataaatctcctgtctgcccatcatatagaagaagctgaagatcagaggtggaaacagcaggatctgtagacccggagagaacccaagctagaggaaccctcactgactggtgcagggatctcacccccatcccctgagctctctgtttaggtatgtgtctttagtatagcatgcttgtgcctcaaactgcaaggaagcaagtggtatgaatctgtgatctgctcagctctggctgtgagcatcagatgtaacaaatcaaagtcaggagaactgcctttagcggtgcacctggacatcagagccccttgtaagaactgggaaattgctggcagtctagtggagcggtacggtaaatctggaaaacactcccctctgtcactgaaggctgtagtagaatccaattaaagctattcaaaccactgccgcagaccctctgggtccctgtgtctgcgtggaatgggtctctcgagcaggtgggcaaagcgtaggattaaactgacaaacagatgcacacaggagaggcgttgaatctgaatgtaatgtcaggtcgaacctcggactttttataatacagaaaaaattccaggggcgatatgtcacaccagacaaggtacactgaaatttaccagatacaagagaatgacttcgaaaggaatttgcaggaactagataaatgtttacaatagagattatcagccctgcctaggatcatcagcctagtgccaggtaagaatttcacactttagtgttaaaagcaccagggggtaattactcttggcaggcctcaagaataatgtagtgtcacggacccctaaattcctaggccttgttaaatcttatcttgtctggagaatccccatttatcaggagagtatctcaacttgctcttggcatggaatgtagcctgtactaaagatttttatctcagtgagattcccaggctctttctgcagaccagatgagctaatggccttgtttattgtaatgcttaattagttactgaataggttaaactccttgctgttatctgggatcttggaacaatgggggcgctgggggggggggcgggggggctttagctatgtcagaattcaatcttaaaaggcacttacaatagggtaatgcttaatagagggcacatggatccatacactagactaacgcgggagtggaaaattaatttatggtttagagggaacgccagactccaggagggaatttctgtgaaactcttttccttgtgggcagcttccaagcttccgcttgtcaaactgacccaactggagagcgtggctttcaccagactccaggagggagctttcgtgaaactcttcctcatgagatagcttccaagctttcgcctgtcaagctgactccaccagagtgcgtggcaaaccaccactgtgaaattcctgtagaattatgattctctctgctcttggacagcagagggtgaggtttgacagatacgactcaggcatcagagaagctgcaggcaggccctctcccatgtctaaacatgagacttggatgtgctcctgggatcaccagcatgctttatactctataaacatagaatccttggagaacattccttagcacccccggaaggcagatgtaaagaggcagactagacgtggaggaagacgctgactgtcaaaactgctgccccagagctaatttcctctcacagtaccatataagtaacaaataataggcaataaaaacctgaaggaactaagagtcacccatggtcctctatctattgttactatcattttatacctgattatttcctatgcatatatatgtaagaaagattacaccctgggttttaaaacacaacagtttttgtaagcagatttagtaaataaacatgtatattatcatttaatggttaaatagcatttcatagtttggttgtatccatttagtattaaattgtttcaaacttccccctactattacactactaaacttgc

SEQ ID NO: 77 - DNA, 745 bp, Homo sapiens
ggaagcctcttttcctccgcagagaggaagcgggagaggagcccacgtcgcctgtcacccaatatctccagccgcgcagtcccgaagagtgtaagatgttcgcctgcgccaagctcgcctgcaccccctctctgatccgagctggatccagagttgcatacagaccaatttctgcatcagtgttatctcgaccagaggctagtaggactggagagggctctacggtatttaatggggcccagaatggtgtgtctcagctaatccaaagggagtttcagaccagtgcaatcagcagagacattgatactgctgccaaatttattggtgcaggtgctgcaacagtaggagtggctggttctggtgctggtattggaacagtctttggcagccttatcattggttatgccagaaacccttcgctgaagcagcagctgttctcatatgctatcctgggatttgccttgtctgaagctatgggtctcttttgtttgatggttgctttcttgattttgtttgccatgtaacaaattactgcttgacatgttggcattcatattaattacggatgtaattctgtgtatcttactgtgactccgaaaactgtagtattggtgtcatgggaatgtacgttatttccaaagtcatttcattaaagatgaaaactttaatttcttctgtgatttgtacttacactaagtttagattatcacaaagaagaacgtgcattcaggcagatgctgtccca

SEQ ID NO: 78 - DNA, 2245 bp, Homo sapiens
agctgttgcagcatgagcgatacgcttggttctcctaactagcaccttcccctctcccctgactcagctggtagcccctcctccccgcacctgcccaaaggtcactggacaggcatttgtctggccttcccttttactgctggctgggaaggaggagcatcagaccacagatcctggaaggcacttctctccctgactgccgctcacactgccgtgagaacctgcttatatccaggaccaaggaggcaatgccaggaagctggtgaagggtttcctctcctccaccatggttgacagcactgagtatgaagtggcctcccagcctgaggtggaaacctcccctttgggtgatggggccagcccagggccggagcaggtgaagctgaagaaggagatctcactgcttaacggcgtgtgcctgattgtggggaacatgatcggctcaggcatctttgtttcccccaagggtgtgctcatatacagtgcctcctttggtctctctctggtcatctgggctgtcgggggcctcttctccgtctttggggccctttgttatgcggaactgggcaccaccattaagaaatctggggccagctatgcctatatcctggaggcctttggaggattccttgctttcatcagactctggacctccctgctcatcattgagcccaccagccaggccatcattgccatcacctttgccaactacatggtacagcctctcttcccgagctgcttcgccccttatgctgccagccgcctgctggctgctgcctgcatctgtctcttaaccttcattaactgtgcctatgtcaaatggggaaccctggtacaagatattttcacctatgctaaagtattggcactgatcgcggtcatcgttgcaggcattgttagacttggccagggagcctctactcattttgagaattcctttgagggttcatcatttgcagtgggtgacattgccctggcactgtactcagctctgttctcctactcaggctgggacaccctcaactatgtcactgaagagatcaagaatcctgagaggaacctgcccctctccattggcatctccatgcccattgtcaccatcatctatatcttgaccaatgtggcctattatactgtgctagacatgagagacatcttggccagtgatgctgttgctgtgacttttgcagatcagatatttggaatatttaactggataattccactgtcagttgcattatcctgttttggtggcctcaatgcctccattgtggctgcttcaaggcttttctttgtgggctcaagagaaggccatctccctgatgccatctgcatgatccatgttgagcggttcacaccagtgccttctctgctcttcaatggtatcatggcattgatctacttgtgcgtggaagacatcttccagctcattaactactacagcttcagctactggttctttgtggggctttctattgtgggtcagctttatctgcgctggaaggagcctgatcgacctcgtcccctcaagctcagcgttttcttcccgattgtcttctgcctctgcaccatcttcctggtggctgttccactttacagtgatactatcaactccctcatcggcattgccattgccctctcaggcctgcccttttacttcctcatcatcagagtgccagaacataagcgaccgctttacctccgaaggatcgtggggtctgccacaaggtacctccaggtcctgtgtatgtcagttgctgcagaaatggatttggaagatggaggagagatgcccaagcaacgggatcccaagtctaactaaacaccatctggaatcctgatgtggaaagcaggggtttctggtctactggctagagctaaggaagttgaaaaggaaagctcacttctttggaggcacctgtccagaagcctggcctaggcagcttcaacctttgaacttactttttgaaatgaaaagtaatttatttgttttgctacatactgttccagacttttaaaggggacaatgaaggtgactgtggggaggagcatgtcaggtttgggcttggttgttttagaagcacctgggtgtgcctacctactcctcttttcttttaaaagggcccacaatgctccaatttcctgtctcctttagagagacatgaaactatcacaggtgctggatgacaataaaagtttatgttcctaaaaaaaaaaaaaaaaaaaaaaaaaaaaaaaaaaaaaa

SEQ ID NO: 79 - DNA, 518 bp, Homo sapiens
cggagaaggcaacgcttctctttctggtcaaaatggctggtaagcaggccgtttcagcatcaggcaagtggctggatggtattcgaaaatggtattacaatgctgcaggattcaataaactggggttaatgcgagatgatacaatatacgaggatgaagatgtaaaagaagccataagaagacttcctgagaacctttataatgacaggatgtttcgcattaagagggcactggacctgaacttgaagcatcagatcttgcctaaagagcagtggaccaaatatgaagaggaaaatttctaccttgaaccgtatctgaaagaggttattcgggaaagaaaagaaagagaagaatgggcaaagaagtaatcatgtagttgaagtctgtggatgcagctgttatgaagatggttaaacttgaaacaaacaattttaagaattatttggtctgaagatgttttactttaaataaatgtctattgtaatggctggagtttttgaattccaaaccttatactg

SEQ ID NO: 80 - DNA, 571 bp, Homo sapiens
agttccggctctgccgtgaagagctttgcattgtgggaagtctttcctttctcgttccccggccatcttagcggctgctgttggttgggggccgtcccgctcctaaggcaggaagatggtggccgcaaagaagacgaaaaagtcgctggagtcgatcaactctaggctccaactcgttatgaaaagtgggaagtacgtcctggggtacaagcagactctgaagatgatcagacaaggcaaagcgaaattggtcattctcgctaacaactgcccagctttgaggaaatctgaaatagagtactatgctatgttggctaaaactggtgtccatcactacagtggcaataatattgaactgggcacagcatgcggaaaatactacagagtgtgcacactggctatcattgatccaggtgactctgacatcattagaagcatgccagaacagactggtgaaaagtaaaccttttcacctacaaaatttcacctgcaaaccttaaacctgcaaaattttcctttaataaaatttgcttgttttaaaaacattgtatctaaaaaaaaaaaaaaaaaa

SEQ ID NO: 81 - DNA, 1401 bp, Homo sapiens
cttgccttccccgtccctcatggcgctgctccgacgcccgacggtgtccagtgatttggagaatattgacacaggagttaattctaaagttaagagtcatgtgactattaggcgaactgttttagaagaaattggaaatagagttacaaccagagcagcacaagtagctaagaaagctcagaacaccaaagttccagttcaacccaccaaaacaacaaatgtcaacaaacaactgaaacctactgcttctgtcaaaccagtacagatggaaaagttggctccaaagggtccttctcccacacctgaggatgtctccatgaaggaagagaatctctgccaagctttttctgatgccttgctctgcaaaatcgaggacattgataacgaagattgggagaaccctcagctctgcagtgactacgttaaggatatctatcagtatctcaggcagctggaggttttgcagtccataaacccacatttcttagatggaagagatataaatggacgcatgcgtgccatcctagtggattggctggtacaagtccactccaagtttaggcttctgcaggagactctgtacatgtgcgttggcattatggatcgatttttacaggttcagccagtttcccggaagaagcttcaattagttgggattactgctctgctcttggcttccaagtatgaggagatgttttctccaaatattgaagactttgtttacatcacagacaatgcttataccagttcccaaatccgagaaatggaaactctaattttgaaagaattgaaatttgagttgggtcgacccttgccactacacttcttaaggcgagcatcaaaagccggggaggttgatgttgaacagcacactttagccaagtatttgatggagctgactctcatcgactatgatatggtgcattatcatccttctaaggtagcagcagctgcttcctgcttgtctcagaaggttctaggacaaggaaaatggaacttaaagcagcagtattacacaggatacacagagaatgaagtattggaagtcatgcagcacatggccaagaatgtggtgaaagtaaatgaaaacttaactaaattcatcgccatcaagaataagtatgcaagcagcaaactcctgaagatcagcatgatccctcagctgaactcaaaagccgtcaaagaccttgcctccccactgataggaaggtcctaggctgccgtgggccctggggatgtgtgcttcattgtgccctttttcttattggtttagaactcttgattttgtacatagtcctctggtctatctcatgaaacctcttctcagaccagttttctaaacatatattgaggaaaaataaagcgattggtttttcttaaggtaaaaaaaaaaaaaaaaaa

SEQ ID NO: 82 - DNA, 2493 bp, Homo sapiens
ggatcgatttgagtaagagcatagctgtcgggagagcccaggattcaacacgggccttgagaaatgtggctcttgtacctcctggtgccggccctgttctgcagggcaggaggctccattcccatccctcagaagttatttggggaggtgacttcccctctgttccccaagccttaccccaacaactttgaaacaaccactgtgatcacagtccccacgggatacagggtgaagctcgtcttccagcagtttgacctggagccttctgaaggctgcttctatgattatgtcaagatctctgctgataagaaaagcctggggaggttctgtgggcaactgggttctccactgggcaaccccccgggaaagaaggaatttatgtcccaagggaacaagatgctgctgaccttccacacagacttctccaacgaggagaatgggaccatcatgttctacaagggcttcctggcctactaccaagctgtggaccttgatgaatgtgcttcccggagcaaatcaggggaggaggatccccagccccagtgccagcacctgtgtcacaactacgttggaggctacttctgttcctgccgtccaggctatgagcttcaggaagacaggcattcctgccaggctgagtgcagcagcgagctgtacacggaggcatcaggctacatctccagcctggagtaccctcggtcctacccccctgacctgcgctgcaactacagcatccgggtggagcggggcctcaccctgcacctcaagttcctggagccttttgatattgatgaccaccagcaagtacactgcccctatgaccagctacagatctatgccaacgggaagaacattggcgagttctgtgggaagcaaaggccccccgacctcgacaccagcagcaatgctgtggatctgctgttcttcacagatgagtcgggggacagccggggctggaagctgcgctacaccaccgagatcatcaagtgcccccagcccaagaccctagacgagttcaccatcatccagaacctgcagcctcagtaccagttccgtgactacttcattgctacctgcaagcaaggctaccagctcatagaggggaaccaggtgctgcattccttcacagctgtctgccaggatgatggcacgtggcatcgtgccatgcccagatgcaagatcaaggactgtgggcagccccgaaacctgcctaatggtgacttccgttacaccaccacaatgggagtgaacacctacaaggcccgtatccagtactactgccatgagccatattacaagatgcagaccagagctggcagcagggagtctgagcaaggggtgtacacctgcacagcacagggcatttggaagaatgaacagaagggagagaagattcctcggtgcttgccagtgtgtgggaagcccgtgaaccccgtggaacagaggcagcgcataatcggagggcaaaaagccaagatgggcaacttcccctggcaggtgttcaccaacatccacgggcgcgggggcggggccctgctgggcgaccgctggatcctcacagctgcccacaccctgtatcccaaggaacacgaagcgcaaagcaacgcctctttggatgtgttcctgggccacacaaatgtggaagagctcatgaagctaggaaatcaccccatccgcagggtcagcgtccacccggactaccgtcaggatgagtcctacaattttgagggggacatcgccctgctggagctggaaaatagtgtcaccctgggtcccaacctcctccccatctgcctccctgacaacgataccttctacgacctgggcttgatgggctatgtcagtggcttcggggtcatggaggagaagattgctcatgacctcaggtttgtccgtctgcccgtagctaatccacaggcctgtgagaactggctccggggaaagaataggatggatgtgttctctcaaaacatgttctgtgctggacacccatctctaaagcaggacgcctgccagggggatagtgggggcgtttttgcagtaagggacccgaacactgatcgctgggtggccacgggcatcgtgtcctggggcatcgggtgcagcaggggctatggcttctacaccaaagtgctcaactacgtggactggatcaagaaagagatggaggaggaggactgagcccagaattcactaggttcgaatccagagagcagtgtggaaaaaaaaaaaacaaaaaacaactgaccagttgttgataaccactaagagtctctattaaaattactgatgcagaaagaccgtgtgtgaaattctctttcctgtagtcccattgatgtactttacctgaaacaacccaaaggcccctttctttcttctgaggattgcagaggatatagttatcaatctctagttgtcactttcctcttccactttgataccattgggtcattgaatataactttttccaaataaagttttatgagaaatgcc

SEQ ID NO: 83 - DNA, 2787 bp, Homo sapiens
aggtttctagacgtgacccagggcagactggtagcaaagcccccacgcccagccaggagcaccgccgaggactccagcacaccgagggacatgctgggcctgcgccccccactgctcgccctggtggggctgctctccctcgggtgcgtcctctctcaggagtgcacgaagttcaaggtcagcagctgccgggaatgcatcgagtcagggcctggctgcacctggtgccagaagctgaacttcacagggccgggggatcctgactccattcgctgcgacacccggccacagctgctcatgaggggctgtgcggctgacgacatcatggaccccacaagcctcgctgaaacccaggaagaccacaatgggggccagaagcagctgtccccacaaaaagtgacgctttacctgcgaccaggccaggcagcagcgttcaacgtgaccttccggcgggccaagggctaccccatcgacctgtactatctgatggacctctcctactccatgcttgatgacctcaggaatgtcaagaagctaggtggcgacctgctccgggccctcaacgagatcaccgagtccggccgcattggcttcgggtccttcgtggacaagaccgtgctgccgttcgtgaacacgcaccctgataagctgcgaaacccatgccccaacaaggagaaagagtgccagcccccgtttgccttcaggcacgtgctgaagctgaccaacaactccaaccagtttcagaccgaggtcgggaagcagctgatttccggaaacctggatgcacccgagggtgggctggacgccatgatgcaggtcgccgcctgcccggaggaaatcggctggcgcaacgtcacgcggctgctggtgtttgccactgatgacggcttccatttcgcgggcgacgggaagctgggcgccatcccgacccccaacgacggccgctgtcacctggaggacaacttgtacaagaggagcaacgaattcgactacccatcggtgggccagctggcgcacaagctggctgaaaacaacatccagcccatcttcgcggtgaccagtaggatggtgaagacctacgagaaactcaccgagatcatccccaagtcagccgtgggggagctgtctgaggactccagcaatgtggtccatctcattaagaatgcttacaataaactctcctccagggtcttcctggatcacaacgccctccccgacaccctgaaagtcacctacgactccttctgcagcaatggagtgacgcacaggaaccagcccagaggtgactgtgatggcgtgcagatcaatgtcccgatcaccttccaggtgaaggtcacggccacagagtgcatccaggagcagtcgtttgtcatccgggcgctgggcttcacggacatagtgaccgtgcaggtccttccccagtgtgagtgccggtgccgggaccagagcagagaccgcagcctctgccatggcaagggcttcttggagtgcggcatctgcaggtgtgacactggctacattgggaaaaactgtgagtgccagacacagggccggagcagccaggagctggaaggaagctgccggaaggacaacaactccatcatctgctcagggctgggggactgtgtctgcgggcagtgcctgtgccacaccagcgacgtccccggcaagctgatatacgggcagtactgcgagtgtgacaccatcaactgtgagcgctacaacggccaggtctgcggcggcccggggagggggctctgcttctgcgggaagtgccgctgccacccgggctttgagggctcagcgtgccagtgcgagaggaccactgagggctgcctgaacccgcggcgtgttgagtgtagtggtcgtggccggtgccgctgcaacgtatgcgagtgccattcaggctaccagctgcctctgtgccaggagtgccccggctgcccctcaccctgtggcaagtacatctcctgcgccgagtgcctgaagttcgaaaagggcccctttgggaagaactgcagcgcggcgtgtccgggcctgcagctgtcgaacaaccccgtgaagggcaggacctgcaaggagagggactcagagggctgctgggtggcctacacgctggagcagcaggacgggatggaccgctacctcatctatgtggatgagagccgagagtgtgtggcaggccccaacatcgccgccatcgtcgggggcaccgtggcaggcatcgtgctgatcggcattctcctgctggtcatctggaaggctctgatccacctgagcgacctccgggagtacaggcgctttgagaaggagaagctcaagtcccagtggaacaatgataatccccttttcaagagcgccaccacgacggtcatgaaccccaagtttgctgagagttaggagcacttggtgaagacaaggccgtcaggacccaccatgtctgccccatcacgcggccgagacatggcttgccacagctcttgaggatgtcaccaattaaccagaaatccagttattttccgccctcaaaatgacagccatggccggccgggtgcttctgggggctcgtcggggggacagctccactctgactggcacagtctttgcatggagacttgaggagggagggcttgaggttggtgaggttaggtgcgtgtttcctgtgcaagtcaggacatcagtctgattaaaggtggtgccaatttatttacatttaaacttgtcagggtataaaatgacatcccattaattatattgttaatcaatcacgtgtatagaaaaaaaata

SEQ ID NO: 84 - DNA, 10482 bp, Homo sapiens
gcgttgcagtccttactgtgtgaaaggcccccgcggcgctcccggccgccgtcttcttcccgcgcccttgggctcgcgtcctgcccgtccgggcctggcgtagcgctgccgggaacccacgcggcgctggcgccgggcgccttcaagggacgcgctcccgtctttctcccgccccgggtttctccgtgaaaaagcgctcggtttgatggccactgtcctaggtttcgtgggaataaagcgcgcagagcccatgactcgggggcaggagaaagggagtcccgaaagcgcgaggcggaaccgcgggagcaggccggcctctgcgagtttgctcacttccgacagagaaaaacaaaaggtgacagttcgcattcggagaaaaagacggcgaagaggaagggctcggctgtcgatgcgtctgtccaggaggagagtccggtaaccaaggaggacagcgcactctgtggaggaggggacatttgcaaaagcacatcatgtgacgacacccctgatggggcaggaggggcctttgcagctcagccggaggactgtgatggagagaagagagaggacttggaacagctgcagcagaagcaagtcaatgaccatcctccagagcagtgtgggatgttcacagtcagtgaccacccaccagaacagcatgggatgttcacagtcggtgaccacccaccagaacagcgtgggatgttcacagtcagtgaccacccaccagaacagcatgggatgttcacagtcagtgaccacccaccagaacagcgtgggatgttcacaatcagtgaccaccaaccggaacagcgtgggatgttcacagtcagtgaccacacaccagaacagcgtgggatcttcacaatcagtgaccacccagcagaacagcgtgggatgttcacaaaggagtgtgaacaagaatgtgaacttgccattactgacctggagagcggccgtgaagatgaggctggcctgcatcagagtcaggccgtgcatggccttgagctggaggcgctgcgcctgagtctgagcaacatgcacacggcgcagctggagctgacacaggccaacctccagaaggagaaggagacggcattgacggagctgcgggagatgctcaacagccggcgtgcccaggagctggccctgctacagagcaggcagcagcacgagctggagctcctcagggagcagcacgcacgggagaaggaggaggtggtgctcaggtgtggacaggaagcagctgagctgaaggagaagttacaatcagaaatggagaaaaacgcccagatagtaaagaccctgaaggaagattgggaatctgaaaaagatttatgtttagaaaatctacgcaaagaactgtctgcaaagcatcaatcagaaatggaggatttacaaaaccagtttcagaaagaattggcagaacagagagctgagttggagaagatttttcaagacaaaaaccaggctgaacgggcccttaggaacctggagagtcatcatcaagcagccattgagaagttacgtgaagacctgcagtccgagcacggccggtgtttagaagacttggagttcaagttcaaagagagcgagaaagaaaaacagctggagttagagaatcttcaagcatcatatgaagacctgaaggcacaatcacaagaagagatcaggcgcttgtggtcccagcttgattctgccaggaccagtagacaggaattgagtgagctacatgagcaactcctggcgcgcacctctcgtgtggaagatttagaacagctgaagcagcgagaaaaaacccagcatgagtcagaactggagcaactgaggatttattttgaaaagaagttaagggatgctgagaaaacttaccaagaagacctaaccctgttacagcagaggctgcagggggcgagggaagatgctcttctggactctgtggaagttgggttgtcctgtgtgggtttagaagagaaacctgagaaaggaagaaaagatcacgttgatgaactcgagcctgagcgacataaggagagcctgccacgcttccaggcggagttagaagaaagccacaggcaccagctggaagcgctggagtctcccctctgcatccagcacgaggggcatgtctcagacagatgctgcgtagagacttcagcattgggacacgagtggcgtctggaaccctctgaagggcacagccaagagcttccctgggtgcatctccagggtgtgcaggacggggacttggaggccgacacagagcgggcagccagagtcttgggtctggaaactgagcacaaggtgcaactttcgcttcttcagactgagctcaaagaagaaattgaactcctaaaaatagaaaatagaaatttgtatgagaagttgcagcatgaaactcgtctgaaggacgatttggagaaggtaaaacacaatctaattgaagaccaccagaaggaactaaataatgctaagcaaaagactgagctgatgaaacaggaattccaaagaaaagaaacggactggaaagttatgaaggaggagctacagcgggaagctgaggagaagttaacattgatgctacttgaactgagagaaaaggctgaatccgagaaacagaccatcataaacaagtttgagcttcgagaagctgaaatgaggcagcttcaggaccaacaggcagcccagatcctggatctggagaggtccttgacggagcagcagggccgcctgcagcagctggaacaggacctcacttcagacgacgccctgcattgcagccagtgtgggcgggagccgcccacagcccaggacggggagcttgccgcgctccacgtgaaggaagactgcgccctgcagctgatgctggcccggagcaggtttttagaggaacgtaaagagatcaccgagaaattcagtgcggaacaagatgccttcctgcaggaggcccaggagcagcatgcccgtgagctgcagctcctccaggagagacaccagcagcagctcctgtcagtgacggcggagctcgaggccagacaccaggccgcgttgggcgagctgacagcctccttagagagcaagcagggggctctgctggctgcacgtgtggccgaactgcagacaaaacacgctgccgacctcggcgctctggagaccagacatctgtccagccttgattctttggaatcctgttacctgtctgaatttcagaccatccgtgaggagcacaggcaggccctagagctcttacgagcagactttgaggaacaactgtggaaaaaggactctcttcaccaaacgattttgactcaagagttggagaaactgaagcggaaacacgaaggggagctacagtctgtgcgggaccacctgcgaaccgaagcgagcacagagctcgccggaaccgtggctcacgagctgcagggagtgcaccagggtgaatttggaagtgaaaagaaaactgctttgcatgaaaaagaggagacacttcggcttcagagtgcacaggcacagccttttcaccaagaggagaaagagtctttgtctctgcagcttcaaaagaagaatcaccaagtccagcagctgaaagaccaggttttatccttaagtcacgagatagaagagtgccgctccgagttggaggtgctgcagcagaggcgggagcgggagaaccgggaaggcgcaaacctcctctccatgctcaaggccgacgtcaacctgtcccacagcgaaagaggggccctccaggacgccctgcgcaggctgctgggtttgtttggagagacgctgagggcagccgtcaccctgaggagccggatcggggagcgcgtggggctctgcctggatgacgcgggcgcaggcctggccctgtcgacagctccggcgctggaggagacatggtctgatgtggccctcccggagttggacagaactttgtctgaatgtgcagagatgtcttccgtggctgaaattagcagccacatgcgtgaaagctttctcatgagcccagaaagtgtgcgggagtgtgagcagcccatccggagggtcttccagagcctcagcctggccgtggacggcctcatggagatggccctggactccagcaggcagctggaagaagcacgccaaattcattctcgttttgaaaaagaatttagttttaagaatgaggagacagcacaggttgtcaggaagcaccaggagctgctggagtgtttgaaggaggagagcgcagcaaaggcagagctggcgctggagctgcacaagactcagggtacccttgagggattcaaggtggagacagcagatctgaaggaggtgctggccgggaaggaggattccgagcaccgtctggtgctggagctggagagcctgagacggcagctgcagcaggcggcccaggagcaggcggcgctgagggaggagtgcacccgtctgtggagtcggggggaggccacagccacggacgccgaggccagagaagctgctctccggaaggaagtggaggatctgaccaaagaacagtcggagaccaggaagcaggctgagaaggaccgctcagccctgctctcccagatgaagattttggagtctgagttagaagaacagctgtctcagcatcgcgggtgtgccaagcaggcggaggccgtcactgccctggaacagcaggtggcatctctggacaagcatttgcgcaaccagcggcaattcatggatgagcaggcagccgagcgggagcacgagcgcgaggagttccagcaggagattcagaggctggaggggcagctccgccaggcggccaagccgcagccctggggccctcgcgacagccagcaggcgccgctggatggagaggttgagttgttacaacaaaagttgagagaaaagttggatgaatttaatgaattggctatacagaaagagtcggcagatagacaagtgttaatgcaggaagaagaaattaaacgtctggaggagatgaacatcaacatcaggaaaaaagtggcccagctccaggaagaagtggaaaaacagaaaaacatcgtgaaagggctggaacaggataaagaggtgttaaagaaacagcagatgagtagcttgcttctggcgtccacgttgcagtctacactagatgcaggcagatgtcccgagcctccttcgggcagccctcctgagggtccagaaatacagttagaggtgacacagagagcactcctgcggcgcgagagcgaggttttggacttaaaagaacagctagaaaagatgaaaggtgacttagaaagtaaaaatgaagaaatactacatctgaacttaaaattggacatgcagaacagccagactgctgtcagcctcagagaacttgaggaagagaacacgagcttgaaggtcatatataccagaagttctgagattgaagagctgaaagccactattgaaaatctgcaagagaatcagaaacgattacaaaaggagaaagcagaggaaattgaacaactccatgaagtcattgagaagctgcagcacgagctgtccctcatggggcctgtggtgcacgaagtcagcgacagtcaggctggcagtctgcagagcgagctgctctgctcccaggccgggggccctcgtgggcaggccctacagggcgagctcgaggctgcgctggaagccaaggaggccctgagccggctgctggctgaccaggagcgcaggcacagccaggccctggaggccctgcagcagcgcctccagggcgcagaggaggctgcggagctacagctggctgagctggagcgcaatgtagccctcagggaggctgaggtcgaagacatggcctcccggatccaggagttcgaagcggccctgaaagcaaaggaagcgacgattgccgagagaaatttagaaatcgacgctctgaaccagcggaaggcggcccactctgccgagctggaggccgtcctgttggccttggcccgcatccgccgcgccctggagcagcagcccctggcagccggggcggcgcctcccgagctgcagtggctccgagcgcagtgtgcccgcctcagccgccagctgcaggtgctgcaccagcggttcctgaggtgccaggtggagctggacaggcggcaggcccgcagagccacagctcacacacgggtgcccggggcccacccacagcctcgcatggatggtggcgccaaggcccaggtcaccggcgacgtggaggcctcccatgatgctgctttggagccggttgtccctgacccacagggtgatctgcagcctgtcctggtgacgttgaaggatgcacctctctgcaagcaagaaggcgtgatgtcagtgctcaccgtctgccagaggcagctgcagtcggagctgctcttggtgaaaaatgaaatgcgcctgagtctggaggacggcggcaagggtaaagaaaaagtactggaagattgtcagctgccgaaggtcgatctcgtagctcaggtgaaacagcttcaggaaaaactgaaccgtttgctgtattccatgaccttccagaatgtggatgctgccgacaccaaatctctgtggcccatggcctcagcacacctgttggagagcagctggagtgatgattcctgtgacggagaagagcctgacatatcaccccacatagacacatgtgatgccaatacagccacggggggtgtaactgatgttatcaaaaatcaggccatagacgcgtgtgatgccaatacaaccccagggggtgtaactgatgttatcaaaaattgggattccttgataccagatgaaatgccagattctcccattcaagaaaaatcagaatgtcaggacatgtctctttcttcaccgaccagcgtacttggtggctcccgccaccagagccacactgcagaggctgggccccggaagagcccggtcgggatgctggacctgtcttcctggagctcccctgaggtcctcaggaaggactggaccctggagccctggcccagcctccccgtgacaccccactcaggagccctgagcctgtgcagtgccgacacatccctgggggacagggcggacacctcgctgccacagacccaggggccggggctgctttgttccccaggcgtgtctgcagcagcgctggcactgcagtgggccgagtctccgccggctgacgaccaccatgtgcagaggacggctgtggagaaagatgtcgaagattttatcacaacatcctttgattctcaagaaacattaagttcacctcctcctggattagaaggaaaagctgatagaagtgagaaaagtgacggctcgggttttggagcaagactgagcccggggtcaggaggccctgaggctcaaactgctggtcctgtgacccctgcttccatctctggaaggtttcagccgctgccggaagccatgaaggagaaggaagtgcgtccgaagcacgtgaaggctttactgcagatggtgcgtgacgagagccaccagatcctggcgctgtcagaaggccttgcacccccaagcggcgagccacacccaccccggaaggaagacgagatacaggacatctcgctccatgggggaaagacgcaggaagtgcccaccgcgtgccccgattggagaggggaccttctgcaggttgtgcaagaggcctttgaaaaagagcaggagatgcagggggttgagctgcagccccgactcagtggctcagatctggggggtcacagctccctgctcgaaaggctggagaagatcatccgtgagcagggagacctgcaggaaaagtccctggagcatcttcgcttgccggaccggagcagcctgctgtccgagatccaggcgctgcgtgcccagctgcgcatgacgcacctgcagaaccaggagaagctgcagcacttgcgcacggcgctgacaagcgcagaggcgcgcgggagccagcaggagcaccagctgcgcaggcaggttgaactgctggcttataaagtagagcaggagaagtgcattgctggtgacttgcagaagacgctgagtgaagagcaagagaaggcaaacagcgtgcagaagctcctggcggcggagcagactgtagtgcgagatttgaagtccgacctctgtgagagcaggcagaagagcgaacagctgtcccggtccctctgcgaggtgcagcaggaggtcctccagctgagatccatgctgagcagtaaggagaacgagctgaaggccgcgcttcaggagctggagagtgagcaggggaaggggcgtgccctgcagagccagctggaggaggagcagctgcggcacctgcagagggagagccagagtgccaaggccctggaggagctgcgggcgtctttggagacacagcgtgctcagagcagtcgactctgcgtggcactgaaacacgagcagacggccaaggacaacctgcagaaggagctgcgtatcgagcactcacgctgcgaggccttgctggctcaggagcggagccagctctctgagctccagaaggaccttgcggctgagaagagccgcaccctggagctgtcagaggccttgcggcacgagcggctcctgaccgagcagctgagccagaggacacaggaggcttgcgtgcaccaggacacacaggcccatcacgctctgctgcagaagctgaaggaggagaagtcccgggtggtggacttgcaagcgatgcttgaaaaggtgcagcagcaagccctgcattctcagcagcagcttgaggctgaggctcagaagcactgtgaggcgctcaggagagagaaggagcgagaattagaactgcagcgtcagcgtgacttgcataagatcaagcagcttcagcagacagtgagagacctggagtcgaaggacgaggtgcctggcagccgcctccacctaggttctgcccgcagggctgccggctcggatgcggaccacctccgggaacagcagcgagagctggaggcgatgaggcagcggctgctctctgccgcccggcttctcaccagcttcaccagccaggccgtggacaggacagttaatgattggacgtcatccaatgagaaagcagtgatgtctttactgcacacgttggaggagctgaagtctgacttgagcaggcccacctcctcccagaaaaaaatggcagcagagctgcagttccagtttgtggacgtcctgctgaaagacaatgtttccctcacaaaagcgctcagcacggtgacccaggagaagctggagctgagcagagccgtgtctaagcttgagaagttgctgaagcaccatctgcagaagggctgcagcccaagcaggtcggaaaggtctgcttggaagccagacgaaacggctccacagagttccctgaggcgcccagaccccggccggcttccaccagctgccagcgaggaagcacacaccagcaatgtcaagatggaaaaattgtacctgcattacttgagagcagagagctttagaaaagctctgatttatcaaaagaagtatcttttgctgttgattggtggattccaggattctgaacaagaaacactctccatgattgcccatttgggggtatttccttccaaagcagaacggaaaatcacatctcgtcctttcaccaggttccgcacggccgtcagggtggtcattgcaatattaagattacgttttttggttaagaaatggcaagaagtagatcggaaaggagctctggcacaaggcaaagcccctcgcccagggccccgagcacgacagccgcagtctccacccagaaccagagagtcccccccaacccgggatgtaccctctggccacaccagggaccctgccagaggccgcagactggcagcagcagcctccccacacagtgggggaagagccactccatccccaaattcaagattagaaagatccctgactgcttctcaagatccagaacattccttgacagagtatattcaccatttagaagtgatccagcaaagattgggaggggtactaccagattctacttcaaagaaatcctgccacccgatgattaaacagtgaataaaatgtcatggctctttcctgcgacaattctatttgaggaaaagatttgtttttcccttttcccaaggaagctcgtgggacagcatgggcactactcttcatgtgcggtgacaccagcccccagatgccttgaattaagtgtcctcacctttatgcatgactgcaaagccagctggagcattttctatggagcctccgtatgttttaggcccatgaccttcgtgaggtgacgggcactcactcccatgagccctggctgtgtgctgttgtgtgcctatcggcagatccatccttcctgcctccaaggaggatacacagagaatggcttcctgttgttttgtttattttcttaacgtgtacagatggaaacttcatttaaaaataaaaacaaaacaactcaaaaaggaataaaatttaatcactgttttgtttgtg

SEQ ID NO: 85 - DNA, 403 bp, Homo sapiens
gctaccatggtctccggcagcaacggcctcgccgccgcccgtctcctgtcgcgcagcttcctcctgcaacagaatggaattcagcattgttcctacacagcttctcggcaacatctctatgttgataaaaatacaaagattatttgccagggtttcactggcaaacagggcacctttcacagccagcaggcattggaatatggcaccaaactcgttggaggaaccactccagggaaaggaggccagacacatctgggcttacctgtctttaatactgtgaaggaggccaaagaacagacaggagcaacggcttctgtcatttatgttcctccgccttttgctgctgctgccattaatgaagctattgaggcagaaattcccttggttgtgtgtatcactgaag

SEQ ID NO: 86 - DNA, 2033 bp, Homo sapiens
cttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttaaaaaaaaaaaaaaaaaaaaaaaaaaaaaaaaaaaaaaaaaaaaaaaaaaaaaaaaaa

SEQ ID NO: 87 - DNA, 668 bp, Homo sapiens
gcagcaagatggcggcggtctcaatgtcagtggtactgaggcagacgttgtggcggagaagggcagtggctgtagctgccctttccgtttccagggttccgaccaggtcgttgaggacttccacatggagattggcacaggaccagactcaagacacacaactcataacagttgatgaaaaattggatatcactactttaactggcgttccagaagagcatataaaaactagaaaagtcaggatctttgttcctgctcgcaataacatgcagtctggagtaaacaacacaaagaaatggaagatggagtttgataccagggagcgatgggaaaatcctttgatgggttgggcatcaacggctgatcccttatccaacatggttctaaccttcagtactaaagaagatgcagtttcctttgcagaaaaaaatggatggagctatgacattgaagagaggaaggttccaaaacccaagtccaagtcttatggtgcaaacttttcttggaacaaaagaacaagagtatccacaaaataggttggcactgactatatctctgcttgactgtgaataaagtcagctatgcagtatttatagtccatgtataataaatacatctcttaatctcctaataaattggacctttaaactacaaaaaaaaaaaaaaaa

SEQ ID NO: 88 - DNA, 2640 bp, Homo sapiens
cggatgggtgctattgtgaggcggttgtggaagagtttcgtgagtgctcgcagctcatacctgtggctgtgtatccgtggccacagctggttggcgtcgccttgaaatcccaggccgtgaggagttagcgagccctgctcacactcggcgctctggttttcggtgggtgtgccctgcacctgcctcttcccccattctcattaataaaggtatccatggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaaagaaatggttggttggtggttttttttagtttgtatgccaagtgagaagatggtatatttggtactgtatttccctctcattttgacctactctcatgctgcagagggtactttaagacatactccttccatcaaatagaaccactatgaagctacctcaaacttccagtcaggtagttgcaattgaattaaattaggaataaataaaaatggatactggtgcagtcattatgagaggcaatgattgttaatttacagctttcatgattagcaagttacagtgatgctgtgctatgaattttcaagtaattgtgaaaaagttaaacattgaagtaatgaatttttatgatattccccccacttaagactgtgtattctagttttgtcaaactgtagaaatgatgatgtggaagaacttaggcatctgtgggcatggtcaaaggctcaaacctttattttagaattgatatacacggatgacttaactgcatttttagaccatttatctgggattatggttttgtgatgtttgtcctgaacacttttgttgtaaaaaaataataataatgtttaatattgagaaagaaactaatattttatgtgagagaaagtgtgagcaaactaacttgacttttaaggctaaaacttaacattcatagaggggtggagttttaactgtaaggtgctacaatgcccctggatctaccagcataaatatcttctgatttgtccctatgcatatcagttgagcttcatataccagcaatatatctgaagagctattatataaaaaccccaaactgttgattattagccaggtaatgtgaataaattctataggaacatatgaaaatacaacttaaataataaacagtggaatataaggaaagcaataaatgaatgggctgagctgcctgtaacttgagagtagatggtttgagcctgagcagagacatgactcagcctgttccatgaaggcagagccatggaccacgcaggaagggcctacagcccatttctccatacgcactggtatgtgtggatgatgctgccagggcgccatcgccaagtaagaaagtgaagcaaatcagaaacttgtgaagtggaaatgttctaaaggtggtgaggcaataaaaatcatagtactctttgtagcaaaattcttaagtatgttattttctgttgaagtttacaatcaaaggaaaatagtaatgttttatactgtttactgaaagaaaaagacctatgagcacataggactctagacggcatccagccggaggccagagctgagccctcagcccgggaggcaggctccaggcctcagcaggtgcggagccgtcactgcaccaagtctcactggctgtcagtatgacatttcacgggagatttcttgttgctcaaaaaatgagctcgcgtttgtcaatgacagtttcttttttcttactagacctgtaacttttgtaaatacacatagcatgtaatggtatcttaaagtgtgtttctatgtgacaattttgtacaaatttgttattttccatttttatttcaaaatatacattcaaacttaaaattaaaaaaaaaaaaaaaa

SEQ ID NO: 89 - DNA, 400 bp, Homo sapiens
gcgagctgagccctggccgccgccacaatgggccgcgagtttgggaatctgacgcggatgcggcatgtgatcagctacagcttgtcaccgttcgagcagcgcgcctatccgcacgtcttcactaaaggaatccccaatgttctgcgccgcattcgggagtctttctttcgcgtggtgccgcagtttgtagtgttttatcttatctacacatgggggactgaagagttcgagagatccaagaggaagaatccagctgcctatgaaaatgacaaatgagcaacgcatccggatgacggttccctgtctctgaaagacctttctctggaagaggagtctgcattgtagtgtctcaaagacacaataaacttcctatggtctgcaaaaaaaaaaaaaaaaaaaa

SEQ ID NO: 90 - DNA, 2002 bp, Homo sapiens
aattccgcggcaaccgctccggcaacgccaaccgctccgctgcgcgcaggctgggctgcaggctctcggctgcagcgctgggctggtgtgcagtggtgcgaccacggctcacggcagcctcagccacccagatgtaagcgatctggttcccacctcagccttccgagtagtggatctaggatctggcttccaacatgtggcagctctgggcctccctctgctgcctgctggtgttggccaatgcccggagcaggccctctttccatcccgtgtcggatgagctggtcaactatgtcaacaaacggaataccacgtggcaggccgggcacaacttctacaacgtggacatgagctacttgaagaggctatgtggtaccttcctgggtgggcccaagccaccccagagagttatgtttaccgaggacctgaagctgcctgcaagcttcgatgcacgggaacaatggccacagtgtcccaccatcaaagagatcagagaccagggctcctgtggctcctgctgggccttcggggctgtggaagccatctctgaccgcatctgcatccacaccaatgcgcacgtcagcgtggaggtgtcggcggaggacctgctcacctgctgtggcagcatgtgtggggacggctgtaatggtggctatcctgctgaagcttggaacttctggacaagaaaaggcctggtttctggtggcctctatgaatcccatgtagggtgcagaccgtactccatccctccctgtgagcaccacgtcaacggctcccggcccccatgcacgggggagggagatacccccaagtgtagcaagatctgtgagcctggctacagcccgacctacaaacaggacaagcactacggatacaattcctacagcgtctccaatagcgagaaggacatcatggccgagatctacaaaaacggccccgtggagggagctttctctgtgtattcggacttcctgctctacaagtcaggagtgtaccaacacgtcaccggagagatgatgggtggccatgccatccgcatcctgggctggggagtggagaatggcacaccctactggctggttgccaactcctggaacactgactggggtgacaatggcttctttaaaatactcagaggacaggatcactgcggaatcgaatcagaagtggtggctggaattccacgcaccgatcagtactgggaaaagatctaatctgccgtgggcctgtcgtgccagtcctgggggcgagatcggggtagaaagtcattttattctttaagttcacgtaagatacaagtttcaggcagggtctgaaggactggattggccaaagtcctccaaggagaccaagtcctggctacatcccagcctgtggttacagtgcagacaggccatgtgagccaccgctgccagcacagagcgtccttccccctgtagactagtgccgtgggagtacctgctgcccagctgctgtggccccctccgtgatccatccatctccagggagcaagacagagacgcaggatggaaagcggagttcctaacaggatgaaagttcccccatcagttcccccagtacctccaagcaagtagctttccacatttgtcacagaaatcagaggagagatggtgttgggagccctttggagaacgccagtctccaggtccccctgcatctatcgagtttgcaatgtcacaacctctctgatcttgtgctcagcatgattctttaatagaagttttatttttcgtgcactctgctaatcatgtgggtgagccagtggaacagcgggagcctgtgctggtttgcagattgcctcctaatgacgcggctcaaaaggaaaccaagtggtcaggagttgtttctgacccactgatctctactaccacaaggaaaatagtttaggagaaaccagcttttactgtttttgaaaaattacagcttcaccctgtcaagttaacaaggaatgcctgtgccaataaaaggtttctccaacttg

SEQ ID NO: 91 - DNA, 2639 bp, Homo sapiens
ctctaaaggccgcgggccggcggctgaggccaccccggcggcggctggagagcgaggaggagcgggtggccccgcgctgcgcccgccctcgcctcacctggcgcaggtggacacctgccgaggtgtgtgccctccggcccctgaagcatggccagcagcggcatggctgacagcgccaaccacctgcccttctttttcggcaacatcacccgggaggaggcagaagattacctggtccaggggggcatgagtgatgggctttatttgctgcgccagagccgcaactacctgggtggcttcgccctgtccgtggcccacgggaggaaggcacaccactacaccatcgagcgggagctgaatggcacctacgccatcgccggtggcaggacccatgccagccccgccgacctctgccactaccactcccaggagtctgatggcctggtctgcctcctcaagaagcccttcaaccggccccaaggggtgcagcccaagactgggccctttgaggatttgaaggaaaacctcatcagggaatatgtgaagcagacatggaacctgcagggtcaggctctggagcaggccatcatcagtcagaagcctcagctggagaagctgatcgctaccacagcccatgaaaaaatgccttggttccatggaaaaatctctcgggaagaatctgagcaaattgtcctgataggatcaaagacaaatggaaagttcctgatccgagccagagacaacaacggctcctacgccctgtgcctgctgcacgaagggaaggtgctgcactatcgcatcgacaaagacaagacagggaagctctccatccccgagggaaagaagttcgacacgctctggcagctagtcgagcattattcttataaagcagatggtttgttaagagttcttactgtcccatgtcaaaaaatcggcacacagggaaatgttaattttggaggccgtccacaacttccaggttcccatcctgcgacttggtcagcgggtggaataatctcaagaatcaaatcatactccttcccaaagcctggccacagaaagtcctcccctgcccaagggaaccggcaagagagtactgtgtcattcaatccgtatgagccagaacttgcaccctgggctgcagacaaaggcccccagagagaagccctacccatggacacagaggtgtacgagagcccctacgcggaccccgaggagatcaggcccaaggaggtttacctggaccgaaagctgctgacgctggaagacaaagaactgggctctggtaattttggaactgtgaaaaagggctactaccaaatgaaaaaagttgtgaaaaccgtggctgtgaaaatactgaaaaacgaggccaatgaccccgctcttaaagatgagttattagcagaagcaaatgtcatgcagcagctggacaacccgtacatcgtgcggatgatcgggatatgcgaggccgagtcctggatgctggttatggagatggcagaacttggtcccctcaataagtatttgcagcagaacagacatgtcaaggataagaacatcatagaactggttcatcaggtttccatgggcatgaagtacttggaggagagcaattttgtgcacagagatctggctgcaagaaatgtgttgctagttacccaacattacgccaagatcagtgatttcggactttccaaagcactgcgtgctgatgaaaactactacaaggcccagacccatggaaagtggcctgtcaagtggtacgctccggaatgcatcaactactacaagttctccagcaaaagcgatgtctggagctttggagtgttgatgtgggaagcattctcctatgggcagaagccatatcgagggatgaaaggaagtgaagtcaccgctatgttagagaaaggagagcggatggggtgccctgcagggtgtccaagagagatgtacgatctcatgaatctgtgctggacatacgatgtggaaaacaggcccggattcgcagcagtggaactgcggctgcgcaattactactatgacgtggtgaactaaccgctcccgcacctgtcggtggctgcctttgatcacaggagcaatcacaggaaaatgtatccagaggaattgattgtcagccacctccctctgccagtcgggagagccaggcttggatggaacatgcccacaacttgtcacccaaagcctgtcccaggactcaccctccacaaagcaaaggcagtcccgggagaaaagacggatggcaggatccaaggggctagctggatttgtttgttttcttgtctgtgtgattttcatacaggttatttttacgatctgtttccaaatccctttcatgtctttccacttctctgggtcccggggtgcatttgttactcatcgggcccagggacattgcagagtggcctagagcactctcaccccaagcggccttttccaaatgcccaaggatgccttagcatgtgactcctgaaggaaggcaaaggcagaggaatttggctgcttctacggccatgagactgatccctggccactgaaaagctttcctgacaataaaaatgttttgaggctttaaaaagaaaaaaaaaaaaaaaaaaaaaaaaaaaaaactttagagcaca

SEQ ID NO: 92 - DNA, 577 bp, Homo sapiens
cgcgcttaagtaactagtccgtagttcgagggtgcgccgtgtccttttgcgttggtaccagcggcgacatgacggggtacactccggatgagaaactgcggctgcagcagctgcgagagctgagaaggcgatggctgaaggaccaggagctgagccctcgggagccggtgctgcccccacagaagatggggcctatggagaaattctggaataaatttttggagaataaatccccttggaggaaaatggtccatggggtatacaaaaagagtatctttgttttcactcatgtacttgtacctgtctggattattcattattacatgaagtatcatgtttctgaaaaaccatatggcatagttgaaaagaagtccagaatattccctggtgatacaattctggagactggagaagtaattccaccaatgaaagaatttcctgatcaacatcattaaagattatgtaaaaagttaaaaggcttatgagcctaagtttgttcctatattaccatatttactgaattttctggaaaagtaactttaataaagtttaatctcagaaattgtcaaaaaaaaaa

SEQ ID NO: 93 - DNA, 23065 bp, Homo sapiens
gatcatggacctcttacacaatttttgcctcatctgtgccctataagaatcatagttattatttaaatttatttttaaaagaaacttggtatcagcctcataaataaacagtatcactttccatatattaaaaaataaaataaaatgaacaaacaatacagttactaaatcctagaaacccaggagtagattggggggataccctggctctgaccttcattctcttcagccttccagtatcaggagattacagaaaagaattaatcctttactcctttcccaaaaaaattgcatctcagtataacatttaaggatggccagacacagtggctcatgcctgtaatcccagcactttgggaggccaaggcaggaggattgtgtgagcctccgagttcaagaccaacctgagcaacacaatgagacctccagctccacaagatattttttgaaaattagccaggcatgggtggtatgtgcctatagtcccagctacttgagaggctgaggcgggaggattgcttaagcccaggagttggaggatgctgtgagctatgatcgagccactgtactccagtctgggcaagagtaagaccctgtctcaaaaacaaaaaaaaataagtttaaggataaccaaatagccctagttgatgagcaaaagctcatttgtacaaaagcatggcagccaatcataaatgttaaaaaaaaattagaaaaataacctctttgcaactcctaataaaataactgactcaggcaaggatgctaaaacacacaggtaaaaggttaatgggaaactagatatataggtatgatttctaagtattaccccaatactcacttgctcttacaagggggaaaataatactttctaatattatttacaagctgtcacctttactcaatttaagtactaccaaacattattggcttccttatatgatgctatatgagatatacaagatcacctataaagaatactataggctgggcatggtggcttgagcctgtaatcctagcactttgggaggccgaggcaggaggagtgcttgagttcaggagtttgagaccagcctgggcaacatggtgagaccctatctctatacaaataaaaaagttaattaggcatggtggcaatcacctgtggtcccagctacttgggaggctgaggcaggaggaggattgcttgagctgggaaggtcaaggttgcagtgagccatgatcgcgccactgtacacgagcctgggctaccaattgagaccttgtctcaaaaaaaaaaaaaaaaatcatattcctccctgcaaatgtttaacctgaaattaatctatcaagatcacctacaaagtattctttctccccaaaatgtttaatctaacccaatcaaagcttttagatataatttccagttgacagaaactacgagttagaaggcaaaagacaaccagaggaaacaagtcaacatatatgggatgctacaggacaacagcctggcctcctctcttcaacaagtcaatgtcaagaaaaagaaggctgggcgcagtggttcacacctgtaatcccagcaccttgggaggccgaggcaggtgaattacctgaggtcgggagtttgagaccagcctggccaacgtggcaaaacttcgcctctaccaaaaatacaaacattagccaggtgtggtgccaggcactgtaatcccagcctgtaaagtagctgggacagctacttgggaggctgaggcatgagaatcacttgaacccgggagcaaaggttgcagtgagctgagatcgcatcactgcactccagcctgtgagacaggtgagactctgtctataaaaaaaaaaaaaagaaaaaggggtggagggaaaatgggttatattctaaattcagaatatagagaaaattaaatttggagaaaactaaaagtcctaataactcagagcacagaggactttttgggcagttaaactattttgtgagattttataatggtgaatacgtatcattatgcatttgtcaaaacccataggatgtacaacaccaagagtgaaccctaatgtaaactatggattctggatgatactgatgtgtcaatgtagtttcatccatggtaacaaatataacacctcgttgcaggatgttgatgatgagggagactggagaggggagcggaggtcctaataaccaaacctataacagtctattgcttttgatcgaatcttgatttcaaggagcagttggaggacaactgaggaagtgtgaatgtgtattagatgataaagaggaacttctattaattgtgttatgttgatgacatatactatggttatatagaaaagtatcctgaaattttagagatgcctatttaagtatttaaaggtgaaatatcctgatattggtatttaccttaaaatattttttaaaaagtaaatacggcaaaattacttaaagtgttaagtctgggtgttacagactgcatgtctgcgtcccctcaaagttcatatattgaaccctaacccccaagggaatggaattaaaaggtgggcctttggagatgactggatttagatgaggtcacaagggtagagcctgcatgatgaacagggtgggaccgtatctcaaaaaaaaaaagaggaagagaagtttctattagagcttgctctatctgctttgtgaggatagaggcagtttcgcaaaccaagaagaggaccctcactagaatctgaccatgccagcaccttgatcttgaacttcccagcctccagaactgtgagaaataaatttctgtcatttaaaccacccagtctactgcactttatatggcagtccaagcagactaagacattagataatgcatatacaggtattccttaatagaactaaagagagtaactgtatcaatattagtatgagtcctacctaaaatacctcttccttcacttgcagaaacactggattagattattccaattgcctcagtgttcttccacccttacccaatccagtccaatctccggagaaaaatatgatcacatcactagcctgcacagcattcaatcaaccacctctttaatggggaaaagtgtaaattccacagtgtagccatataaggcttttcgagatctgcccagactccttgagccactctatatatacttcactcatttaaaactcagcgacaatccatcaagtaggtgaaattattattccctctttacagataagtaaaggggaatataagtaaataacttgcccaagaccacacggttggtaagtggccctgcttgactttaaattttgacagtctgatatatccataaaccagataaatccataattttaaccacaatattattctgtgcccggtacagtgtaggtgctcaataaatgcttgctagatgagtagtgtggaagagcttagctctcctttttcacctgatcccaaaagaggagtgaatcctaaccaccctgtttctcttaagagtcaagtcaagtcagagctgatgactgttactggcattgcccgggcgtctagtattgcagtatccccgccctgtgaggggtgcaaaaccttttctgatttcatttctttggtggtggggatcggactggggaggaatgaggggtcgcagaacacaggctgcccgctgggcccgggggctgcggggagggacggctctcgtgcgctgagtagttgactcgcccactgcagacctcagaagcctcacgacccggaccccggagccccagccccgcgccctcagccccgcgtccaggacctgatcctggattgccaggggccctcctgggaagacggggccactccgggcccactcacctcgtcccggcactgcttctggcacatctggcaataccagcgtagcttctgcagccccttggacttgatcctgttggcgatagccttgggagtaagaaaatccgacttccccatggcgaccacggcagcgatcactttctggaccccagtactcagccagccggaaacggaaccgggctggcggcggtgggcgggacaaatcccagctgagaggcggaaggaggtggggcctgggaagactgggttgcaacggtcgcgagactcccctcgccgtagcgcgatgaaacgagactgaagaagagagcaaggtgggaggggcgcgctggggagcttcggcgcatgcgcgctgaggcctgcctgaccgaccttcagcagggctgtggctaccatgttctctcgcgcgggtgtcgctgggctgtcggcctggaccttgcagccgcaatggtatggcagcttgcgggaagatcggcaggaccgcaaggatggaagagcttgggcacggggcagggaggaggagatgggcgcgggggcagatctggggtcgcagggcctagctgggccctggccgtcggaggcgccgggatgcggggctgcaggaagcggctctgaggtcgggcaaggtgtgcgcagggccgggctccggcggagcaaggtcccgcccggttccctgaggtcggagggccacctgggacctcgggactgcggcctgagtccaaattgacgtctgggcggtcttgagttccctcagctaatcagcacttcctgagcttccacttccactgcaagccaggcccccagtctcagggcctgcaaggtgacgggagcctgagcaatagcccaggggaaggttaagtcgtgctttttgtccttgacgccctttgaaaccctctagtgctggacagagtgaccctttaagattcgatcgagtcagagagcgcggccgtcgagcacctcccgtgttgcagacgctttgctgggtgctgcaacagcaaaggcgaacatcctagatgcctctgagggtggttttggtgcagaagggaccgtacagatctgtctgagggcggggaagggaaggacacctagcactaccttgaaaccacttgctgaaaccgtaaccctgcaaggtcatgaaattgcccccaggtcctgggggaggagttcggtgggcttttaagtggcatttgttaactgacatgcaactaaagggtgccacgagcctcgagaattttaaacattagttccgggtagggggacctgggctgggggaagtgcatattagaaggcccgggaatgggcaggtaaaagttggtttgtgggtggtgctgttcagttttgaagcttgacggaatctggaagaattcaatccctgccgtctgaatgcagcccttcactggctacagagtctctctcttcatacagtactacactctgctgcctcacattttttacatttattagtaaatatctgccctcatggtctcagatgacaatttgtcttttctctcctgaaatgccatctccttatcggtatctgtagtttatgactactgacacgttattaaaaaacagttccattttagagactactgaactctaagaaatagaaatgttttccaaatttgaagacagtaaaatccgggatcttctctcaaatttttaatttccattttcttatatgctgctttaaaaaaaggtttaattttcaaaacgttagagtattttgccgataccctaacaactattaaaataaccactgtgaatttaagtaagcctaatgtttttatgttagcatggtagcctctctgaaaacaaaaactaaaataacaaattgtactttaaaaaacttccagtaatcaagacagaagcatggaaatatgcctcttcatggttcgttaagacaaaggtcttttcttccccttccaaattaattctgtggagtggttttaatttctgaattctgattgccacttagcagctctgtgaccttgggcaggttactcagtgtctgtcccaccagttcctcatctgtcaaatatatgcaataaaagtgcacaattcagtgggttagtgtgaggattagagtatttgcaaagcatgtaggacaatgggagaaatgctgatagggatccaaatgctgtatatcagtagagaaatgaagagtttttaagtacagtttctacatcaaatgtaatgtaagaagattgcttggacttgcgaaaagaggaaaagaagatgcagtcgggctaggcgcagtggctcacgtctgtaatcccagcattttgagaggccgaggcgggcggatcacaaggtcaggagatcaagaccatactggccaacatggtgaaaccctgtctgtactaaaaatacaaaaattacccagacgtggtggtacgcacctgtagtcccagctactcgggaggctgaggcaggagaattgcttgaaccccgggaggcggaggttgcagtgagctgagatcgtgccactgcatttcagcctggcgacacagtgagactccatctcaaaaacaaaaaaaaagatgatgcagtcatggtgtgggttaggtaagtgtgaaggagtcagtttttgctggtggcaggtaatggacttttacacgcttaatatcagaattagagtcacttcataattttcgttgagaattagccaatattgcttgctttttaagtttttatgtatcttaatatactccttaaaaatctctcaaacaatagtttcttggaagggtgtcattcctgactccctcctccactgccttcgtcagctgttataacagcgctgtggaatcatggtagatagtggtaatccacggtccatttgattctccctccactgtgtttcttagtctcccgaggatctcaggacgtgcaatgattcagagctttatctcctcatttgcaaaacttttatatttaataaaggaagctatccctaaagagagttcaattagctcatcactgttcttactcaaattgtacctaacaattaaagtagtaacatagacgtcttcagaattaaatgaaagctaaacatgagagtgctttaaaacacatgaaattagggcaaatgcgtagcattatcacaaaagatgctacctcttcatacctctgacttggagtacttaaggaaaccaaaacattctgtgtttgtccaaatatggtattcaattcaaatttataccatatggtattctctgactagaaacttgtgtcagttgaagtgtctgcctaaaacaataatatccaagagtgacttgttataaccaataaattttgtgttcagggagaaaagtcattactacaccaaaaaaggaaggtttgaccaaaaactggtattacttttcaagtgtgattatgaaaagctgttgtagtagaacagtcatggcaggtagtttgtgctctgaaataacagagcttgtcaggatttgccagcttttatcttttcctttagtagattcattcttaaaaccaaacctaatatttttcgtgctgtagtgggagaatcacctgagcccaggaagttgagactgcaggaagccatgatcacaccactgcactatagcctgggtgaggggagtgagaccctgtcttcaaaccgcaaaaaacaaagatccactctttttgttatacagttccacggatttggacaaatgcataatgacatgtatccaccattacaatatcatgccaaagagcttcactgccctaaaaatccctcctctactctccccaaaaccctgacaaccacttctctcaactattgctagatcctcggcttttctggcatgtcacatagccagaatcatacagtatttgctttttttcagatggccttctctcccttagcagtatgcacttaaggtttctccatgcctttttataacttaatagctcctttgtggttttggtgaataatagtccattgtatggatgtatcagagttagattatccattcatgtattgaaggacatctgggttgcttccaacttttggcatcatgaatgaagctgctttacatgctcttgtgtaggttttatgtggacataacatttcatctcagttgggtaagttaagactaggtttaggtttgtaaaaagctaccagaacatcttcccaagtggctgtgtactattttacattctgccagccatcaatgagagttccttttgctccacatcctcatcatcatttgctggtcagtttcttgggttttatccattcttgcaggtgtgtagtgatatcttactgttttaacttgcagttctctaatgacatgatactgagcatcatcttatgtgctttttgccatctggttggtttttgtttttgttttttgagacagagtcttgctctgtcatccaggtggaatgcaatggcacgatctctgctcaccacaacctccacctcctgggttcaagcgattcttctgcctcagcctcctgagtagctgggattacaggtgcccaccagcacgcctggctaattttttgtatttttagtagagacggggtttcaccatgttggtcaggctggtctcggaactcctgacctcgtgatccgcctgcctcggcctcccaaagtgctgggattataggcgtgagccaccgcaccggccctgccatctgttgatcttctttggtgaagtttccagatcttttgcccattttttttatttgggttgtttgttctcttattgcattttaagagttgtttgttttggatacaagtcctttatcagagatgagttttagaaatattttctcccagcctgtggattgacttttcattctcttcacagtatttttcacaggacagaagttctaaatttcaataaagcccaactcatttttttctgtcatggatcatgcttttggtgttgtgtttaaaaactcattgccaaacccaaggcacctggattgtctcctgtggtatcttgtagaagttttatggtttggcattttatatttaggcctgtgattcattttaagttaatttttattaaatttgtaaagtcattgtacagattaatatttttgcatattagtgtccagttgttccagtgccatttgtttgttgaaaagaatatccttttttccttgaattgcttttgcttttttattactgtaactttagaggaggtcttaacgtcaggtagtgtcagtcctgacaatattgtttgacttttctaggtctttggccttcctatataaccttgaaaatcagtttattgatatccacaaaatagatatcaactaaaatcaaacttgctgagattctaatggatttttaaaattgtaaaacacatggaacatgaagtttatcattttaaccatacaattgtgtgcgggcatttttaacttcatactccagtagttcagggctgctgcagaggaatgcaactagctttttatatacactaaccttatatcctgcaaccttgtcataaatgcttattggttctaggaggggctatttttattctttgggattttctacatagataatcatgtcatctatgaacaaaaacagttttattccttcccttcaaatttctatgcctcttattttcttgtcttactgcattagctaagacttccagtatgatactgaagaagagtgatgagtggggatttccttatcttgttcgcaatcttatggggaaagctcctagcctctgggattaaatcagagattttgtgtgttgttttttttttttagcaggttgaggaagttctctattcctagtttgctgagagactttatcatgactgggtgttagattttgttggatgctttttctgcacctattgattgtatgattttttctttaacctgttgacgtaatagattatattatttgattttttaatattgaaccagctttgcatacctggagtaaatcccacgtagtgatggtgtataattatttttaatacattgttggatttgatttgctaatacactgttgaggatttttgcctctaacctatgctcatgagagatattagtcactgtccaccaaggtcattcaattagagaaaccttagatcattcttactgctttcctttctactacgaatgtacatgtgaagaagttaaatgcttgcaaatctagtctcctattgctttgccagcatcttcatccaggttcttgaggtctctcctgaggctgtggcagcctacatccccatgtcttctgtgttcatcaaaactccaacctcattttacgcttgtcactgccatgtgagatactttgctgaaatactaaactgatttgtgggcttataaacattcatttccctattaacctacaaataagatttaaaccaatttgctttttttgatatttaaaaatattaattatttaaagtaacaaacccaactcatgtgtccagtttccccttctccatttctcactctttcctgaacatgaatgctcttaaaatgacattctgttcccgagaatcccctcctcacatctctactttgtgcacacatttcatcatgctcagttctcctgtgtagctttgcctgcaaacacaggcactttgtatggatttgaaggcagaaagatcatctaatattttttagcatgtcttcccccattaggccaggaattctctcatattcatctttcaccaacatttcatttaagcctaatcattttgacttctgcacattagcatttcaattttaaagacttgaaaaaaaatactaaatgttatgtaaatgtaattttttatgaaccttgtttacaaggtaacaatatgtcagagaccatgtcgtgtttaccagtgcctggaatttagagcatcttggtaaatgtttattgagctaaattgaaaaagttaagaatcagtttatataaactacttttacacgtgtagtcatgttcgactatatcattgactctgttactggaagaggttgctgtgaactaaggaaatttttagagtggctggccttgattcgaagtatagttgggcagcttaacaaggggaggggcttttctgtcatggaaacaagataagcaaagatatggaggtgggactagagcatgtgtgagtcaatagagcatgttgacttctgggaaggtggtggtacagacaggccaaataaaaggaggccttcaatacagggattcgcgccactactaagggatttaaatatatgtctattcatatacattctgagtcctgtatttgatagattgtttctttatggggtattctttaaataaatatatattaactgaaagtttttcttgtataatggaactatttttcaaaaaactgaaacatttttaaaacttttatcctcaggattcaagttcgaaatatggcaactttgaaagatagtaagtatgttgtttgtctcaatatgtgaattgagaaaaactaaattttgacaaatacaaatactttcaaaaatatactgctttagcccgttgtgtatttgatcaaaactgaccaaatggatttccattgtatgtttgggacattggacttgttctgaaaagaccagtaagaaaggacacctgagacagctatgttctgttgtttgtagaatgatcatgatataactagaaaaaattataccagtgttggtgtaggtgggtgtatgtgtgggaaggggtaaatcatgttatatataaataatgttatggaatatgtggttaaggcaactttcaataccatttaatgttttaaattgtatattattgtcttatgttgccatttgcttgtcctaagtagtagctagttttgccttccttttattagtactatttctttttttttttttttggagatggagcctcattctatcacccaggctggagtgcagtggcgccatctcggctcactgcaagctccgcctcccgggttcacgccgttctcctgcctcagccgttctcctgcctcagcctcccgagtagctgggactacaggcgcccgccaccacgctcggctaattttttgtatttttagtggagacggggtttcaccgtgttagccaagatggtcttgatctcctgacctcatgatccgcccacctcagcctcccaaagtgctgggattacaggcgtgagccaccgcgcccggctattagtactagttctaatattaagactggtttagcatcgtgctgtttattgtttatctaagcgctcattattctgccttgcactttagaaaaatatgtcaataaattcacaaggaagttatactgtaattcctttgtcttaacacagtacttctatttttcagtcaccaggagactaaagtccatcaaaaacatccagaaaattaccaagtctatgaaaatggtagcggcagcaaaatatgcccgagctgagagagagctgaaaccagctcgaatatatggattgggatctttaggtaagggaagagtgtaattcacaaattaggaagaactgtttcacacaaggaagactactgatgacttacatttagagcttacagtgatgtcaagcctatctgagcacttgccatgtacttatttttcatctacatccacttgacacgtaatgataacaacccgtgtaggtagtacaggtagggactgttagaatccctgctttccagtgagaaaaactgaagcacacagaagttaagtaacttgcctaagatcacatataaaaggatgaagagcctctgtttggacctatgtggtttggtgtcgagtctgtgtatttgttagccattataccgtgaccatgttaacttttaaaatattctgaaatgttcccagttgttaagtataccatcagaagaacttggaaaccgtgcgttagatgtaagggcttaatttcatcctatgaccttgtagtgtggcattttaggactgcagcgggggcgggtgaggggcagtgcagtggagagctggccttgtggtcatggatatgactcatgatggtgttttgagaatccgaacgatgttgaagcaggaaaagcagctgagaacctccattttcaagatagttaatcaccattcttatcttttcaaaagcatgaagacatttttccttcagaaatttatgtaatccatccttaattagtcttcttaatggaaaagagatgagatatgacagtttaaaaagaatttatattgtgtttttagatgctttatacttgtttgattatgaatttatcttggaaataaagttgtaaagagctggaaatgggaaagaagtttctattttaacttgtaattacattctttctcttaacttttttatgatcatgctgtcttatcctgagttgaggctatctaaaagaatttttaacttaaggaagatttatgcatatttgatagtaaagaagatcatgcagtgaaccccctatttatctaccaaatcctcagtatctcaactgacagctgtcctttgtttttttatgtgtgtgtgtgtgttttgttgttgttttttgtttgttttctttttgtgtttttttttgagatggagtctcattctgttgcccaggctagagtgcaatggtgcaatctcagctcactacaacctccccctcccaggctcaagcgattctcctgcctcagcctccccagtagctgggattaacaggcccccaccatcacacctggctaatttttgtattttttttttctgagtcggagtctagctctgtctcccgggatggagtgcagtggcataatctcagcttgctgcaagctctgcctcccgggttcacgccattctcctgcctcagcctcccaagtagctggggctacaggcgcccgccaccacgcctggctaatttttttgtattttttagtagagacggggtttcaccgtggtcttgatatcgtgacctcgtgatccgcccgccttggcctcccaaagtgctgggattacagacgtgagccaccgtgcccagccaatttttatatttttaatagagacgggatttcactacgttggccaggccggtctcaaactcctgacctcaggtgatccgcccacctcggcctcccaaagtgctgggattacaggcgtgagccactgtgcccggcctcagcttacagctgtcctaagaacacctgcttacactaggcagttgccagctaaatttaaatgcgtattttggaaataaccaactgctttgtaaattagagatttattgtatttagtgtattgcatataaaaaaattactgcttttgtttgtttttaagctctgtatgaaaaagctgatatcaaggggcctgaagacaagaagaaacacctccttattggtgtgtcctcagatcgaggactgtgtggtgctattcattcctccattgctaaacagatgaaaagcgaggttgctacactaacagcagctgggaaagaagttatgcttgttggaattggtgacaaaatcagaggcatactttataggtaatttaaatatatattgtttattttcataaagatgtaagcacgaataaatcttctctcaaaagttttgacaaactctcaaaaccttcatcaataacaaggtatattcttcagggccttttttggtatattcacaagggggtgtttgtttcctttttcttttttccaggatatattggaacatttctgttttccttcccctgttttctacctttgcttcttcatccggatcataagttgaatgccatgtgaatatcaaaagctatcagcattaagccaaatgaggcatctgaggagctgttacagcctggaaatgttagcaatagttcattaggattagaaggagaggaataaaggggaaaagaatataaaacagacctcatttttagttcttagttctttgcaggtgctgaaggaaaggaattatgatagtgtgtggaacaactcaggaaatcaccaccacccataccccaaaagacctgatccatggtgacaatgttttctcctgcctgtcccctgtgctctttcagcagaaatcttattaaactagctatgtgagctcttgcttttcttataggactcattctgaccagtttctggtggcattcaaagaagtgggaagaaagccccccacttttggagatgcgtcagtcattgcccttgaattactaaattctggatatgaatttgatgaaggctccatcatctttaataaattcaggcaagacagatttagaaatcaaagcttttttatgttcatgcttttgttcatattttgaaataacagtttaaaaattattttgagatttacatttgtttttgtcaattctaggtctgtcatctcctataagacagaagaaaagcccatcttttcccttaataccgttgcaagtgctggtaagtagtttttctatgatacatattttttggcatatagaatggagagatttgtacactaaggcagacagtgtcaagatatctggtggtagctatagcaaatgatttggggctgtttcttttttagatggaatcttgctgcctcccaggctggagtgcagtggcaccatctcggctcactgcaagttccgcctcctgggttcacaccattctcctgcctcagcctccctagtacctgggactacagcacccgccaccaagcctggctaattttttgtatttttagtagagacggggtttcaccatgttagccaggatggtctcaatctcctgtcctcgtgatccacccgcctcggcctcccaaagtgctgggattacaggcatgagcccagccttttatttttttttttttttttttttgagacggagtctcactgtgtcacccaggctgaagtgcaatggcatgatctcggctcactgcaaccttcgcctcccgggttcatgccgttctcctgcctcagcctcccaagtagctgggattacaggtgcacgccaccacgcctggctaatttttgtatttttagtagagacagcgtttcaccgtgttcgccaggctggtcttgaactcctgccctcaagtgatccacctgccttggccttccaaagtgctgggattacaggcatgagccactgcacccggccgatctggggctgtttctgagaagtgccatctcttgacttccatgatacttaattcagtcattgtttgataactcctgctctattctgtcattaattgaaaagtcagtatctattgaggttcatcattagatttttttgtcctcaaggattgaagagcgtagctagaaactggctttgcaatttatgtttgggacctctaactacaataaaacagaagctctagaatagagagttgaacagtgttcacagctaatttacattgactcaattctcttgttttattgttggaataaagtcatcattgatcattgaagcatcctggaataatattctggaactgattttagttttctaaggcttttttcccttgtatataccatctattttctaatacttcatttattttcaagtatgtactttacacgttaatgtttctgaaattgagatggctctaccagccaatggaacatcatttataattgagtacatttttttctctcttaatgagaaataaagtaggtgttacaaccagtaccatttaatgaaatatagtattttgataatatatagtactgtgtcaccacttgaccccaatgaatgaattttgaaatgtacttctaaatttctacaacattaaaactacgattcataccaatttctagttctcccactctttcatgtgctttgaaaggttttccctctctcattttccagattgatacattcagatttcttcagtaatcaaatgactaagttggaattactgcacaggttgagtatcccttgtgtgaatgcttaggaccagaaatgtttgggatttcagaatattttcatatttataatgcagtatcttggggacaggacctgagtctgtacaccctatacacatagcctgaaggcaattttatgcaatattttaaagaattttctgcatgaaacaaagtttcaaatgcaccccatcacacaaggtcaggtgtggaattttccacttgtgcatcacgtcagtctcaaaatgtttcagattttggagcacttcagattttggactttgggattagggatgctcaacctgtattacactaaattcgtttcaattcattattatcttttttactggaaaaagcattgagtgcttgctggctttgtaacaggcacatgctaatgctgtccagagggcttcataaccacgagatatagtgcagttatttcaaatgttgacctaatttttctaaagttacacatgtggagtagaatatcaaatggtttatttacacattatattttgctaaaaatatgctatcatctgtagtgtggaaatagatgacattattagaacagattcatttaccttatctagcattaatttctgctctgtaattataaaggagttttacactgaaaagctgtttttgatgaattactattccttctatataacttgaaagaaactcatcacttgttttaacagacagcatgagtatctatgacgatattgatgctgacgtgctgcaaaattaccaagaatacaatctggccaacatcatctactactctctgaaggagtccaccactagtgagcagagtgccaggatgacagccatggacaatgccagcaagaatgcttgtaagtacacagtatggacagtgccagcaggagtgcttgtgagcacacagtgaatctcagctgagaggagtccgtggccgagagtagcatcaacaacattaacatgatattcctgtacctgtaagaaagtatatcttgataggtagttgatttgcaagtactgtgaaaaggtatcctatggaagatgtaaaaatatcttcataacattatttgagaggccaattttggtatccaaaaatttcttacttttaaattaagcaacagaagttagttaacaagttattctactctagtttcagattttttatgtagtgtttttgttttgtttgtagctgagatgattgacaaattgacattgacattcaaccgtacccgccaagctgtcatcacaaaagagttgattgaaattatctctggtgctgcagctctgtgagtaattgtagattgtcgccttcagagaattttttttcctcttgctgtgtctgcttgtttggatgcttaaaagtttttcgtttttgtcttttgaagttgaaactcattgctgaaacttgcatcctaactcttaggctataaagatgtggtaaaaatgggacctctttgctttatttttattgtttcagctggaaacactcgggcttagttttcggttttctaaaggattaggaatgggatgtggataattcagtatcatttatgaaatctttttttggaatttaaacaattgtaagccaagctttctgagttacagggctagtttcagatttggttttacataaatctataaaccattgcatgttgggaaggtccaggaattttttaatcattatgttttttaatcattatgttttctatatcagtaccaaaagatagtatagccatccctcagtatctgcagtggattggttccagaactcactctcacctcccccaggataccaaaatctgaagatcctcaagtccctgaaataaaatagcacagtactacatataaaaaatgcacatcctctcgtatacttttaatcatctctagttataatgctatgtaaattgttgttgtattggtttttgtttttattattttatcattatttttgtcttttttccaaatattttacatcggctgttggttgaatccatggatactgagggccaactatatctgctttcctcaagaaaatttcttagttgttgaaaaactttgatgtttatgaaaaaaaggttaagataatgggaattttattcaaaaagttattaagttgaatacccactatgtgcacagtacagtgctagacacagtgttaaggatgtgattctctcttttaaggcacttatattctaataggataagaaacatacaagtaattgtgattagataaaacatgtagtaagttctttttgactcgttccttcttacctcctgctaatcagttaccatgctctttaagttttaccttctaactatctcttgaatcttcccccttctttatgcccacttaatgcagatccttgtcatttctctcctgggttcaaaaaggtaattatttgagtgccccttaagtgtcaggccctgtgctagacactgaggatacagtggtaacagtatttgccttcagagagctcagtcttcacagtaaaggcagacactaaataagaagtcacagcagaacagaggttgctaaactgtggcctggagctaagaatgggttctgcatttttaaagggatataagaaaaaggagagtgatagcgtgtgacccacagagcgcagaatacttgctgtgtggcccttcaccgaaaaaatttgcctattcttgtgacagtgttatgaggaaaataacaaaatccattctctgttatacactcattctgcagaaacactcgatgtatgtgatcctatattttctggtcaccagcactcttcccagtccagtctcttaccattaccatttctcacgtccttgaacctcagccaccttgatctacttccagatctacagattcgtcactctttctcgcggccagggagtggcatgtgctgcagcctgaaaagccactatttcttcccttgctcccttgattcttcatctgactgcctcctgtgtatccttccttactcagctgggggaatcctttcttccagaaaacactccttccttcagtctgagtgaggcagccttctgtagcatcctgtcttccatttagcttagctcttacagccttgtatgaggaattggctttttatttgtctgtgtgctcctcgaggctaggatgggaccatatcttattcacttttgtgtccccattgcctggtacatacctccttcgtgaatgttgaatgaatgaatgaaatgccatatgtattcagaactggaagttcagaagagggacaaataacattttattgggtgtatgaaaatgtgggtccatggttttaagagtcagggaaaagttcatcactatttaaagtgttccttaagtgttacttttcaagggcagaaactggcagaatcgagagagacacaactagaactttttgagccaacagagacctttgtgtaagttgttagttggatccatcagccttgctatcctaagcattcttctcttattttactttctaagcacagctgtgttaggactaacccattcacagcctggtcaccaatgttctatcagcatcacagaatactgtctcatggtgtctagcttgatgaacttggttaggatactctctctgttactcagctttgtcatctgtaacatggggactaatacaaggactaaaaccttctattagccattatccttcttgccatctaccccatgttaaagccaggctgatttgttcttggatcttaagtgtgacaaagcctgcgggaaggaaccacattctgaaggtttgtgggctgtgtgagatccagagaacccaagggggtttttttgctcttgacaaacgaatgttaggtatgacctatgatgatacaacttctgcaaaattgaggaccagcctatttcttcattagaaatgcaggaaacctggccgggcgcagtggctgacgcctgtaatctcagcactttgggaggcagaggtgggcggatcacaaggtcaggagattgagaccatcctggctaactcggtgaaaccccgtctctactaaaaatacaaaaaattagctgggcgtggtggtgggcgcctgtagtcccagctactatggaggctgaggcaggagaatggcatgaacctgggaggcggagcttgcagtgagccgagattgtgccactgcactccagcctgggcaaaagagcaagactccgtctcaaaaaaaaaaaaaaaaaaaagaaatgcaggaaacctatttaaaagtaacagaaacagctgggtgcattggctcatgcttgtaatcccagtattttgggagaccgaagcgagtggattgcttgagcccaggagttcaagacaagcctgagcaacatagcaagaccccatctctacaaaaatgaaaaaattagccaggcctggtggcatgtgcctgttgtcctagctactcaggaaattgaggcaggaggattgcttgagcccaggagttttaggctgcagtgagctatgatcgtgccactgcacaaaaccaagaatttagaaaaaggaatccacataattggaaattgagtattaagtttttgcttcctaggaaggtagtctaaatccttatacaaaaacttagctatgcagtgccaaggttggattgcttgtgaatgggtttgcatgctctgtaacctatcagttattctgaatcaagacttgaatatttaaacatacttggaaaagaaaaagaacaaaaaacaaaataaacatatttggctatttagtaatataattaagaacatgagtaaaccatagtattcgaggtccaagtattagttgccagcctaaaaactaaatatggccaggcgcagtggctcatgcctgtaatcccagcactttgggaggccgaggcgggtggatcacctgaggtcaggagttccagaccagcctggccaacatggcaaaaccccgtctctactaaaaaagtacaaaaattagctgggcatggtggcgggcacctgtaatcccagctactcgggaggctgaggcaggagaatgacttgaacccaggaggaggttgcagtgagctgagatcacgccactgcactccagcctgggcaacagaacgagactccatctccaaaaaaaaaaaaaaacctctaaatatattgtgttaatttatagaaaaattgtaatttaaattacatattcatagtcttgcctacttgcatgagcatgtttttattgcttctaacctggattgaaatctatgactttttataacagtcattgagtctgtttcaaaggaatcttgtggtaagtatttttgcatgatgtactcaaggtatcaagtttaacaattgactttttttcattggaaagcatatgtgattaactagcatggattgcttcttgacctgcttgttttgtgtttgtctttttcttctaatataataaccagggattaatgaaaatcaagttccatcctcagacaagaggtaaagttcacacattcttcccatgtctgttcagaaaagaaacctattcagatataagttaagctaatttagtaatcctataattgtcctctgagagatttagatggatgggccacttcacagtagcagccaactcacgggagcacctgggcaacgctagtggacttttttgtagtgagtgtgttgatcatagaaaatgtagatataaggaaataaactttgaggatagagaattaacattattttgagatattatatataggttgtgaagagtggtggttgtcacactttatggtctctggaccccttaatgtctgattcatgtagcagaagccagctagattttcatctgtctctattcattttgttgtgatgtcatggatcatgtggcctctggaaaactctactgtatactcgagaatgagaatataacaggcaaaataacattatcatgaaaatagttttgacctcatgaaccccatgaaaggttccccagaccaaaattttagaatcactggtatagggtaacactttattgtgtaaattcagttctctgtaccccacttaaatatgtattattatctcttgacattattttcccaaaaaatgctgtttgatttcttacttgttctgtactttgtttttcaggtaaagaaggaaaattcagccagttgattttgtttttagcttactgctgcctttgtccgaagaaactgttcctccattatttgaattactgaagacagcaagatatttgtaaattatcttaaaataaacaacttaaaataaaatcattgtttttcttatatataagaacaatagatatagtttttgaaatgagatgatactaaaacatttaaaaatattaatatgctactattaaaattttttagtagaagacaatttaagaggctcttttgatttggtagtgctccactgaccttaaaatgtctagcctctatggggacaacgtagagggaaagggctgcagccacagcctc

SEQ ID NO: 94 - DNA, 2676 bp, Homo sapiens
  modified_base (2022)..(2022): a, c, t, g, unknown or other
  modified_base (2057)..(2057): a, c, t, g, unknown or other
  modified_base (2101)..(2101): a, c, t, g, unknown or other
  modified_base (2104)..(2104): a, c, t, g, unknown or other
gatttgtggagggttatgggagggctctggcgtcctggatggaggtgcgttcctttctgtggctggcgctggatccaccctgggtctccaaccagggctgcagagagggtagagccgtttcttaggccagagtggagtgggacagggggtgccgagagaggactgaggtggcttgggacatggaagcgctgcagccttcgagcccggcatccagcattgcagccgccgcggcggcctaagagctcgaaccctttcacacgcgcgcaggaggaggagcggcggcggcagaacaagacgaccctcacttacgtggccgctgtcgccgtgggcatgctgggggcgtcctacgctgccgtacccctttatcggctctattgccagactactggacttggaggatcagcagttgcaggtcatgcctcagacaagattgaaaacatggtgcctgttaaagatcgaatcattaaaattagctttaatgcagatgtgcatgcaagtctccagtggaactttagacctcagcaaacagaaatatatgtggtgccaggagagactgcactggcgttttacagagctaagaatcctactgacaaaccagtaattggaatttctacatacaatattgttccatttgaagctggacagtatttcaataaaatacagtgcttctgttttgaagaacaaaggcttaatccccaagaggaagtagatatgccagtgtttttctacattgatcctgaatttgctgaagatccaagaatgattaaagttgatcttatcactctttcttacactttttttgaagcaaaggaagggcacaagttgccagttccaggatataattgaagtcagcaactaagtcttccttcaaagttgtgatttttgggaaaatcatatatcctatcttctcaaaggagaaatattgtacaataatatgaaggcttatattttaaataattattttttctcaactaatttttttcacttaaacttgagagaacaagttcagcttttatcataagaaactcatatgcctagctagaatatatgactgactattcaataccatactgaagagtttgatactattaaaataagccgctttgttttttaaatatgcaggcatgggttcagcttaattccataattcccttccagattattaactcttcatacttacagcaatgaatctgacaatgtttttcaaaaatgtatgctcagggttatttgtttcaagtcataggccagaacttctaaccatgtttattgtgtctaaaagagttggttgctattttccttcattatgaatagtcagtattttaaaagctcaaagtagaagaggagtcactaagccctaacagcttgtccaaagatttaaattcttatttcaaatttgatttgtcctttaaggtttagggctattttatattcagagttctgaataattactttgaaactgtagtatcataaaattgaaataaaatacatctactaggctgttagccaaagcatcaccattacaagtccatgaatgttaaaatgtacaggtgggattgtgaagatttactgacatcaaaagttcttctggaaacaaacctttgttaaaaaaaaaaagtattaaattgttttaatagtgatttatttgtcatcaaatgtacaacttattctaaatattttcattttctgtgttctaaatagaaatattaagttgcagtaaaaagagaaaaaaagctatttagcattacaaagaatcatatttaaagggctgcccaatgtagagtctagtgacctgttcaggacacctgaaatataattaaatgacaattatcaaggttttaacaatttataattctaaaccagaggattataaagaatgcaaattgacttttacattcaactttagttaaatgaaggcctcagtattcttcctgaataatacatccagtttctcacattttagctttcatctattcagaattattcatagtaaaataatctactcttatcacagctgtgttgacnatttctaaatgtaggaagcctgtgaaacatgacangcagttaaattggttggcctaaggactaattattttcctctgcnganttttagtgagtatttgttccaaacaagttctgttgaaatctcacgctgttgtcaggaatcagtgttatcctggaactgttattctatttaatcttcattatagcagaaatgtgccaccatggctttgacatgttggtaggtattgtcttccaggcttcaaagctgcacagagtctacgttttagagagttggcacctttgatgtggtagtgagctgatcatccactttcttctaaaataaagagaagaaaatggccagtattcatcctgtccttctctttccccattccttgataactctcttgtctttactgggaagctggagagtcacagtctagtagaagaaagattagaaaaatttcaactgtaggtgcctttgcaacctatcctgggagaccctaaaacatatttagttttagtgcctagagattttcactcctctaacatctctggatacagtgtccctggtcaacatgaagaagttacagaagaacgctttctgatcctggcccctaaagaatttacttgtgctaagtaataaaattcctattgatcaaaaaaaaaaaaaaaaaa

SEQ ID NO: 95 - DNA, 3487 bp, Homo sapiens
cgcggaagggcagaatgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccattttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgctgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgctgtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaagccagaaacctgtgtcatcttttcaccccaccttcaatcaacaagtcctcaatcaacaagtcctactgactgcacatcttaaatatatctttatcagtccacaagtccttccaattatatttcccaagtatatctagaacttatccacttatatccccactgctactaccttagtttagggctatattctcttgaaaaaaagtgtccttacttcctgccaatccccaagtcatcttccagagtaaaatgcaaatcccatcaggccacttggatgaaaacccttcaaggattactggatagaattcaggctttcccctccagcccccaatcatagctcacaaaccttccttgctatttgttcttaagtaaaaaatcatttttcctcctccctccccaaaccccaaggaactctcactcttgctcaagctgttccgtccccttaccacccctgatacaactgccaggttaatttccagaattcttgcaagactcagttcagaagtcaccttctttcgtgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcactgcgccccctgtgcctacctctagcattgtaacgaccacatagtattgaaatggccagtttacttgtctgccttcctttccaagaccgttggtgcctagaggactagaatcgtgtcctatttaactttgtgttcccaggtcctagctcaggagttggcaaataagaattaaatgtctgctacaccgaaacaaa

SEQ ID NO: 96 - DNA, 3254 bp, Homo sapiens
caccgtccgcagcccgagcgccccggagccgcaggcgccgccgcgcagagacgccgcggctgcgactaggcgcgcccagccgcacgtggcggacccgcccccaggcccgcagtgtcctggaccccgcaggcctccgctctcctgtcctcggccccgtccccagggccgcgatgagcttcctgagccgacagcagccgccgccaccccgccgcgccggggcggcctgcaccttgcggcagaagctgatcttctcgccctgcagcgactgtgaggaggaggaagaagaggaggaggaggagggcagcggccacagcaccggggaggactcggcctttcaagagcccgactcgccgctgccgcccgagcggagccccacggagcccgggcccgagcgccgccgctcgcccgggccggcccccggcagccccggcgagctggaggaggacctgttgctgcccggcgcctgcccgggcgcggacgaggcgggcggtggggcggagggcgactcgtgggaggaggagggcttcggctcctcgtcgccggtcaagtcgccggcggccccctacttcctgggtagctctttctcgccggtgcgctgcggcggcccaggagatgcgtcgccgcggggttgcggggcgcgccgggcgggcgaaggccgccgctcgccgcggccggaccacccgggcaccccgccacacaagaccttccgcaagctgcgactcttcgacaccccgcacacgcccaagagtttgctctccaaagctcggggaattgattccagctctgttaaactccggggtagttctctcttcatggatacagaaaaatcaggaaaaagggaatttgatgtgcgacagactcctcaagtgaatattaatccttttactccggattctttgttgcttcattcctcaggacagtgtcgtcgtagaaagagaacgtattggaatgattcctgtggtgaagacatggaagccagtgattatgagcttgaagatgaaacaagacctgctaagagaattacaattactgaaagcaatatgaagtcccggtatacaacagaatttcatgagctagagaaaatcggctctggagaatttggttctgtatttaagtgtgtgaagaggctggatggatgcatttatgccattaagcgatcaaaaaagccattggcgggctctgttgatgagcagaacgctttgagagaagtatatgctcatgcagtgcttggacagcattctcatgtagttcgatatttctctgcgtgggcagaagatgatcatatgcttatacagaatgaatattgtaatggtggaagtttagctgatgctataagtgaaaactacagaatcatgagttactttaaagaagcagagttgaaggatctccttttgcaagttggccgaggcttgaggtatattcattcaatgtctttggttcacatggatataaaacctagtaatattttcatatctcgaacctcaatcccaaatgctgcctctgaagaaggagacgaagatgattgggcatccaacaaagttatgtttaaaataggtgatcttgggcatgtaacaaggatctccagtccacaagttgaggagggcgatagtcgttttcttgcaaatgaagttttacaggagaattatacccatctaccaaaagcagatatttttgcgcttgccctcacagtggtatgtgctgctggtgctgaacctcttccgagaaatggagatcaatggcatgaaatcagacagggtagattacctcggataccacaagtgctttcccaagaatttacagagttgctaaaagttatgattcatccagatccagagagaagaccttcagcaatggcactggtaaagcattcagtattgctgtccgcttctagaaagagtgcagaacaattacgaatagaattgaatgccgaaaagttcaaaaattcacttttacaaaaagaactcaagaaagcacagatggcaaaagctgcagctgaggaaagagcactcttcactgaccggatggccactaggtccaccacccagagtaatagaacatctcgacttattggaaagaaaatgaaccgctctgtcagccttactatatactgagctactcctttcccacctccccctgaacactgtgacaagaggaagctaggttgaaatcactgatagaatccagtttgcaattactttctcgattggtgtcagtagttttactgattaggacttttattgtgaattacagttgaaagctgtattttgatgattgctatgtcaggctttcatctaatcttaccagtctgtcttctgtaggatgtgtcactgttggatgttacaccagcctttccagggttaaccactgtggtggtgtgctgcttatagtttgctgttgcattgtaataaaaggtgtctttccctgtagtgacctgtaaaaagtactcaagggctttattacagacataccctccctttgaaaagggacatgctaaaagactcattactactcagccttcaatgtacctgtgtgtccatcttatatttcttttttttttaattgtgaattagacttgtatatcccactgggagcactttgtaggcattgcatgaaccatgggatgatgattctgtggaggtattgccttgtgaatttgctgctattttagttttgtctttgctgtaaacttgtagcattaaacaatcattgttgttaataggtcttctttttgaaacaattatgtgaaatgtatagctgcttttgatggaaagcagctatttgcctttttttttttcctttgaactttgaagctagtgcattggaaaaatgcaccctttccctcctttggaatgctgtattaatgtagtataataattactggttttgtaacttgttctggtaatgtccttcccggactctttttaaatgtctccccctaagttttatacttgattgtattattagtctgtttttaaatgttttgcccggtttttctcttcaatatttgtgtatataaaccgatcttcgtgatactgtacatagctgtttgaaatgccagaatgacttctgacattccaagtttttcacaaaatatattttatctgtgattagccatttgactaataatactggctaacagatgttgaaaaaaattgtctgttttctcattaattttggtctaaaacatgtttgcacttgtctttgacttgtgttttattaacattgattggcatattaaaagtcactctgagctt

SEQ ID NO: 97 - DNA, 482 bp, Homo sapiens
tttttaggcccatttgaattttttggtgacattcagccgggggttcggggggacggacctccattccaaaaccatgggccaatttgtccgtaaccttgtggaaaaaaccccggcgctggtgaacgctgctgtgacttactcgaagcctcgattggccacattttggtactacgccaaggttgagctggttcctcccacccctgctgagatccctaaagctattcagagcctgaaaaaaatagtcaatagtgctcaaactggtagcttcaaacagctcacagttaaggaagctgtgctgaatggtttggtggccactgaggtgttgatgtggttttatgtcggagagattataggcaagcggggcatcattggctatgatgtttgaagaccaatctttaacatctgattatatttgatttattatttgagtgttgttggaccatgtgtgatcagactgctatctgaataaaataagatttgtc

SEQ ID NO: 98 - DNA, 1642 bp, Homo sapiens
ctctaaaggttcgggggtggaatccttgggccgctgggcaagcggcgagacctggccagggccagcgagccgaggacagagggcgcacggagggccgggccgcagccccggccgcttgcagaccccgccatggacccgttcctggtgctgctgcactcggtgtcgtccagcctgtcgagcagcgagctgaccgagctcaagttcctatgcctcgggcgcgtgggcaagcgcaagctggagcgcgtgcagagcggcctagacctcttctccatgctgctggagcagaacgacctggagcccgggcacaccgagctcctgcgcgagctgctcgcctccctgcggcgccacgacctgctgcggcgcgtcgacgacttcgaggcgggggcggcggccggggccgcgcctggggaagaagacctgtgtgcagcatttaacgtcatatgtgataatgtggggaaagattggagaaggctggctcgtcagctcaaagtctcagacaccaagatcgacagcatcgaggacagatacccccgcaacctgacagagcgtgtgcgggagtcactgagaatctggaagaacacagagaaggagaacgcaacagtggcccacctggtgggggctctcaggtcctgccagatgaacctggtggctgacctggtacaagaggttcagcaggcccgtgacctccagaacaggagtggggccatgtccccgatgtcatggaactcagacgcatctacctccgaagcgtcctgatgggccgctgctttgcgctggtggaccacaggcatctacacagcctggactttggttctctccaggaaggtagcccagcactgtgaagacccagcaggaagccaggctgagtgagccacagaccacctgcttctgaactcaagctgcgtttattaatgcctctcccgcaccaggccgggcttgggccctgcacagatatttccatttcttcctcactatgacactgagcaagatcttgtctccactaaatgagctcctgcgggagtagttggaaagttggaaccgtgtccagcacagaaggaatctgtgcagatgagcagtcacactgttactccacagcggaggagaccagctcagaggcccaggaatcggagcgaagcagagaggtggagaactgggatttgaacccccgccatccttcaccagagcccatgctcaaccactgtggcgttctgctgcccctgcagttggcagaaaggatgttttgtcccatttccttggaggccaccgggacagacctggacactagggtcaggcggggtgctgtggtggggagaggcatggctggggtgggggtggggagacctggttggccgtggtccagctcttggcccctgtgtgagttgagtctcctctctgagactgctaagtaggggcagtgatggttgccaggacgaattgagataatatctgtgaggtgctgatgagtgattgacacacagcactctctaaatcttccttgtgaggattatgggtcctgcaattctacagtttcttactgttttgtatcaaaatcactatctttctgataacagaattgccaaggcagcgggatctcgtatctttaaaaagcagtcctcttattcctaaggtaatcctattaaaa

SEQ ID NO: 99 - DNA, 3481 bp, Homo sapiens
ctcttacaacattggctgccggcgtgactttgaccgcttcccggtgcgttaccggcagctgaacccacccggcgccacgggactttgacgcgtgctctgcgcttgccatgagactcctgggagctgcagccgtcgcggctctggggcgcggaagggcccccgcctccctaggctggcagaggaagcaggttaattggaaggcctgccgatggtcttcatcaggggtgattcctaatgaaaagatacgaaatattggaatctcagctcacattgattctgggaaaactacattaacagaacgagtcctttactacactggcagaattgcaaagatgcatgaggtgaaaggtaaagatggagttggtgctgtcatggattccatggaactagagagacaaagaggaatcactattcagtcagcagccacttacaccatgtggaaagatgtcaatattaacattatagatactcctgggcatgtggacttcacaatagaagtggaaagggccctgagagtgttggatggtgcagtccttgttctctgtgctgttggaggggtacagtgccagaccatgactgtcaatcgtcagatgaagcgctacaacgttccgtttctaacttttattaacaaattggaccgaatgggctccaacccagccagggccctgcagcaaatgaggtctaaactaaatcataatgcagcgtttatgcagatacccatgggtttggagggtaattttaaaggtattgtagatcttattgaggaacgagccatctattttgatggagactttggtcagattgttcgatatggtgagattccagctgaattaagggcggcggccactgaccaccggcaggagctaattgaatgtgttgccaattcagatgaacagcttggtgagatgtttctggaagaaaaaatcccctcgatttctgatttaaagctagcaattcgaagagctactctgaaaagatcatttactcccgtatttttgggaagcgccttgaagaacaaaggagttcagcctcttttagatgctgttttagaatacctcccaaatccatctgaagtccagaactatgctattctcaataaagaggatgactcaaaagagaaaaccaaaatcctaatgaactccagtagagacaattcccacccatttgtaggcctggcttttaaactggaggtaggtcgatttggacaattaacttatgttcgctgttatcagggagagctaaagaagggtgacaccatctataacacaaggacaagaaagaaagtacggttgcaacggctggctcgcatgcatgccgacatgatggaggatgttgaggaagtatatgccggagacatctgtgcattgtttggcattgactgtgctagtggagacacattcacagacaaagccaacagcggcctttctatggagtcaattcatgttcctgatcctgtcatttcaatagcaatgaagccttctaacaagaacgatctggaaaaattttcaaaaggtattggcaggtttacaagagaagatcccacatttaaagtatactttgacactgagaacaaagagacagttatatctggaatgggagaattacacctggaaatctatgctcagaggctggaaagagagtatggctgtccttgtatcacaggaaagccaaaagttgcctttcgagagaccattactgcccctgtcccgtttgactttacacataaaaaacaatcaggtggtgcaggccagtatggaaaagtaataggtgtcctggagcctctggacccagaggactacactaaattggaattttcagatgaaacattcggatcaaatatgccaaagcagtttgtgcctgctgtagaaaaggggtttttagatgcctgcgagaagggccctctttctggtcacaagctctctgggctccggtttgtcctgcaagatggagcacaccacatggttgattctaatgaaatctctttcatccgagcaggagaaggtgctcttaaacaagccttggcaaatgcaacattatgtattcttgaacctattatggctgtggaagttgtagctccaaatgaatttcagggacaagtaattgcaggaattaaccgacgccatggggtaatcactgggcaagatggagttgaggactattttacactgtatgcagatgtccctctaaatgatatgtttggttattccactgaacttaggtcatgcacagagggaaagggagaatacacaatggagtatagcaggtatcagccatgtttaccatccacacaagaagacgtcattaataagtatttggaagctacaggtcaacttcctgttaaaaaaggaaaagccaagaactaactttgcttactgtgagttgactgactctaattgaatctgcgtggttttgatactttgatggattccagtggaataaattcaggctgctgaaacaagaaattctgagcccaggaagcgggctcttctttcttcaaaagaagcccttcttgttcatattcaggagcttctgttatattcaaaggtaattctatgtctatctcaactctattgattggttttatagttcattgaaaatcctcaaataaaatataattattactgaaatatgtttaatatttaaggggaaaagagactaatttcagttatacttttaagcttagaatgtatgttcatttccaaattttgtatcataagagttttcaacatagagaaaagctgaaaaaatgcaaagaataaccacatactttccatctaccttcctttgttaacgggttgtttatcatataataatttgttttgtcatatttgctttcactgtctattatctgtttaagtctcataactctatttttagtttgctgaagacttgaaagtgaatcgcatatatcatgacacttcttggagtgtcattaatgggcaggcttttctgttgaagagtggattccgtatgttcttcatagagagtgtttttcagattcttcattgggatattaaaatattagccaaatttctctctgttttatatatgtctgtttatttcagtttgtggtttctgcaaatttgtaactgcctctgttttaggagtataagtattacttccttgtggtctattgtgaagtaaaaagtagacccttgcatatactattcttgtttgtgttcatcttaatgtttttgtacagctaaatcaaatgtaatttatagagttagtttcatcaacctaatgaatgctagttaaatttgaattccttggaatttatcgtatattgtattcactgagattatgaagggacaaatgttaatcttttgtttccagaaaaagttgggctttcccaagcagttctattacccggttcagaattgcttcatccaaaaatcatctgatggtatagatggatcctagtccttttcattacctgatggtagaaataaaataattgattttaaaaaaaaaaaaaaaa

SEQ ID NO: 100 - DNA, 4299 bp, Mus musculus
tgtgcagtcagatagcagtcatgatcctgagttccctttacccagggaaaagtcagtgctcagcaactcgtccaatgcacttcactacttcattttggctggaccagacaggatttgttggtggctcgcagatcatcagtcctggagcctttggattccatctcagtgtgcttgactgagccatggagtctttcgatgctgacaccaattcaactgacctacactcacggcctctgtttcaaccccaagacattgcctccatggtcattcttggtctcacttgtctattgggactgctaggcaatgggctggtgctgtgggtagctggcgtaaagatgaagacgaccgtgaacacagtctggttcctccatctcaccctggccgatttcctctgctgcctctccttgcccttctccttggctcacctgattctccaaggacactggccctatggcttgttcctgtgcaaacttatcccatccatcattattctcaacatgtttgccagtgtcttcctgcttactgccattagcctggaccgatgtctgatagtacataagccaatctggtgccagaatcatcgaaacgtgagaaccgccttcgccatctgtggatgtgtctgggtggtagcctttgtgatgtgtgtgcccgtatttgtataccgtgatctgttcattatggacaatcgcagtatatgtagatataattttgattcctccaggtcatatgattattgggactacgtgtacaaactaagtctaccagaaagcaattctactgataactccactgctcagctaactggacatatgaatgacaggtcagctccttcctctgtacaggcaagggattacttttggacagttaccactgccctccagtcacagccattcctaacatctcctgaagactcattctctctagattcagcaaaccaacaaccccattatggtggaaagcctcctaatgtcctcacagccgccgtacccagcgggtttcctgttgaagatcgtaaatccaatacactgaacgctgacgcttttctctctgctcacacagaacttttccctactgcttctagtggtcatttatacccctatgatttccagggggattatgttgaccaattcacgtatgacaatcatgtgccgacaccgctgatggcaataaccatcacaaggctggtggtgggcttcctggtgccgtttttcatcatggtaatttgttacagcctcatcgtcttcagaatgcgaaaaaccaacttcaccaagtctcggaacaaaacctttcgggtggctgtggctgtggtcactgtcttttttatctgctggactccataccatcttgtcggagtcctgctattgattactgatccagaaagttccttgggggaagctgtgatgtcctgggaccacatgtccattgctttagcatctgccaatagttgcttcaaccctttcctgtatgccctcttggggaaagactttaggaagaaagcaagacagtctataaagggcattctggaagcagccttcagcgaagagctcacgcactctaccaactgtacccaagacaaagcctcttcaaaaagaaacaatatgagtacagatgtgtgaagatgtggccctgggaacctaagcagagttctcaggtgaacagtgatggatgacatgtgagcaggacactttagacaatttggcgactctcagagaaaggtctcttattgacatcagcatcatttgaaaacattaaagatgcaaaatttcaagccccatcccagatgtgttgactcagaatctctggcccatgggaccagtgttttaacaggccttcttgtttccatcagtgttaagttttacctcatttggcttagtctattcccatccctgactacaccatgtgcaatgaataactttttcatctgttttcagtattctttttttttccttagcatcatctaaacttctagtttgcatggaaggctgctcttattgttctgaatggaagatattcatttattgtacagttttgtggtggtgacaagtgatttttaagtggggaaagagacacagtaagaaaagatctatgaaagcagggagtgttgagttagagtttgacagaacacagtgccaaatgccacccactaaaagcaacctgagataattccagtgttcatgtgagcaagtgagcacagatacacataaacactttcctactcctggagtgttttagaagttgtagcttggagctctctcttccctgtgtattctgcttgttcatagagccatctatgttccatttctttttgtttgttggtttggcttcttgtgttggttggttggttggttggttggttggttggttggttggttgcttggttggttggttggttggttggttggttgcttggttgcttggtttactgttttttcaagacagtagccctggctgtcctgaaactcattctgtagaccaaactggcctgaaactcagagatcagtgtgcctctgcctccaaggagtcttcttaaggtcttgaattcctttctgttttccacacagggccttgccatctcctgctgggattaggcgtgtgcaggtgtgcagtacgtacctggccccatcgtcttcggctttttacattagactttgtatctcacatagaataaaacaatatcctaatgggatgatgggtggaacttcaggatcaatgcatgcttcaacagcaatatcaaaagattttaaacatgagtctaggtatcttacatgacaaaagtgtcaggcacactctcttttttttcttttcttttcttttctttttttttttttcaaattaatgtttgcctggaccatgctaatataaagtactgtatgcagccaattttaaaggaactaattaaaaatgaaataaaaaagggtggtttctccagtgcccagtagttcccatctctccctactttgcaagaattgtagctgttccttctgatatttaccatggtgtgcctaaaacacctcttatgctgctgatctttgtagacaggaagaaaaggctgtcttgattttgatttactttacctacattttcatccagcaccccagtgttgactggtatcacacattaatctgttcgtgatcatttttctcttccctttgttgtcgggttttgcttgccttcgtgccatcatctcagtgtagcacacacaatctagcttcagactcccaggtctcttgttatagtaaccagatgagcaccaccaccatccccacccccagctgaccacacaattttcctgtcaatacatttcaatagcatacagcttttctctgtcctctcatttttcatgctgacatatattaattgtacatattaataaggttccacgtggctgatccatccatgcacacaacattcacactttcacccccagcctcttctttatcattattctacttactgattgactttaaaaaaaaaaaaaaaagtctgaagaatctcatgctggccttaaaatcactatgcaaccaaaaatgagtttgaaattcaaatcctcctgcctcccatgggctgtgattataggcatggaacactatgcccactcttatgtggtactgggactcaaaaccagggcttcatgcatagtcgttaagcatcctaccaactaaactactctgcacccgccccccagcctgctcaggttgacatttctggccctcatatatgagactatgtgatacgtatgtgtccattcacatccattctgttaaaaccagtatttcactcattgttatggctgaataacactgcactgtgtatgtgcatgcacacgcatgtgtgtacatgtgtatgtgcacgtgtgtgtagtaagtgccattatccacccaactaccaaccaatgcctaagcagattctatatcttagctattatacatagtactacactcaacctttagtttaaaaaaagaaaaccagcagctcccataataatctattatgtggatactttccatgtgttaaatcactagaaagtcaagcttgtttgtacagataggtatgtctaagatgaacgagttaatctacaaagacggaagatggaatgtagtttagtcttgtgatacattatttattcagttgtcaagagaagggccttaggctacaactgttgtatgaacaattatgtggaaatttcaaatcactaaaagatcttgtttactacgaggtaattctgggcataagactgcagttctgcctttccagtatgagcggtttacatattccctatgaataaatgcttttatctac

SEQ ID NO: 101 - DNA, 935 bp, Homo sapiens
ccaggcccagctcagctgcaccgggcccccagccatccctggcatcccgggtatccctgggacacctggccccgatggccaacctgggaccccagggataaaaggagagaaagggcttccagggctggctggagaccatggtgagttcggagagaagggagacccagggattcctgggaatccaggaaaagtcggccccaagggccccatgggccctaaaggtggcccaggggcccctggagccccaggccccaaaggtgaatcgggagactacaaggccacccagaaaatcgccttctctgccacaagaaccatcaacgtccccctgcgccgggaccagaccatccgcttcgaccacgtgatcaccaacatgaacaacaattatgagccccgcagtggcaagttcacctgcaaggtgcccggtctctactacttcacctaccacgccagctctcgagggaacctgtgcgtgaacctcatgcgtggccgggagcgtgcacagaaggtggtcaccttctgtgactatgcctacaacaccttccaggtcaccaccggtggcatggtcctcaagctggagcagggggagaacgtcttcctgcaggccaccgacaagaactcactactgggcatggagggtgccaacagcatcttttccgggttcctgctctttccagatatggaggcctgacctgtgggctgcttcacatccaccccggctccccctgccagcaacgctcactctacccccaacaccaccccttgcccagccaatggacacagtagggcttggtgaatgctgctgagtgaatgagtaaataaactcttcaaggccaaggaacagtggtctaattcaactctgtgtcccagcactggcacaccagaagtgccatgctcagaaatgttggttacatgaatgaatgaaccatgaatgaatg

SEQ ID NO: 102 - DNA, 1082 bp, Mus musculus
atgaaacttggcagtgtcctggtgacacgcctttgctgacgggtgtcccaccatcagcaaagggctggagcatccagtttgatcggaccacggaggcagggacaccatggagacctctcagggatggctggtggcctgtgtgctgaccatgaccctagtatggacagtggctgaagatgtctgccgagcacccaacgggaaggatggggctccaggaaatcctggccgcccggggaggccgggtctcaaaggagagagaggggagccaggagctgctggcatccggactggtatccgaggttttaaaggagacccaggggaatctggcccccctggcaaacctggcaatgtggggctcccaggtcccagtggtcccctgggggacagcggcccccaaggactgaagggcgtgaaaggcaatccaggcaatatcagggaccagccccggccagctttctcagccattcggcagaacccaatgacgcttggcaacgtggttatctttgacaaggtcctcaccaaccaggagagtccataccagaaccacacgggtcgcttcatctgtgcagtgcccggcttctattacttcaacttccaagtgatctccaagtgggacctttgtctgtttatcaagtcttcctccgggggccagcccagggattccctgagtttctctaacaccaacaacaaggggctctttcaggtgttagcagggggcaccgtgcttcagctgcgacgaggggacgaggtgtggatcgaaaaggaccccgcaaagggtcgcatttaccagggcactgaagccgacagcatcttcagcggattcctcattttcccctcggcctgagctggggatctgcccctgcatcctgccatctcctgcgctccctgttgtggaccacgcccccccctccgtcctgcacccctcccctccgcactctgcaaagtgaaggggctggggctttagcaccctgggggaggggctgtctcggagggagggcactgaggactgatgtttctgcacactgcccagtggtttctttaagcactttctggaataaacgatccccgtctgtgtctgtgac

SEQ ID NO: 103 - DNA, 4998 bp, Homo sapiens
  modified_base (4581)..(4581): a, c, t, g, unknown or other
  modified_base (4874)..(4874): a, c, t, g, unknown or other
  modified_base (4913)..(4914): a, c, t, g, unknown or other
atgaggacacttgggacttgcctggcgactttggccggacttttgctaactgcggcgggcgagacgttctcaggtggctgcctctttgatgagccgtatagcacatgtggatatagtcaatctgaaggtgatgacttcaattgggagcaagtgaacaccttgactaaaccgacttctgatccatggatgccatcaggttctctcatgctggtgaatgcctctgggagacctgaggggcagagagcccacctgctcttaccccaacttaaggaaaatgacacccactgcatcgattttcactattttgtgtccagcaagagtaattctcctccggggttactcaatgtctacgtgaaggtcaataacgggccactggggaatcctatctggaatatatctggagacccaacacgtacatggaacagggcagaactggccattagtactttctggcctaacttttatcaggtgatttttgaagtgataacttctggacatcaaggctatctcgctatcgatgaggtgaaggtgttaggacatccatgtaccaggactcctcacttcctgcggattcagaatgtggaagttaatgctggccagtttgctaccttccagtgcagtgccatcggcaggaccgtggcaggagacaggctctggttacagggcattgatgtgcgagatgctcctctgaaggaaatcaaggtgaccagctcccgacgcttcattgcttcatttaatgttgtgaataccaccaaacgagatgctggaaagtaccgctgcatgattcgcactgaaggaggtgttggaatatcaaactatgcagagttggtagttaaagaaccacccgttcctattgccccacctcagctcgcctctgtaggagccacctacctgtggatacagctcaacgccaactccatcaatggggatgggcccattgtggcccgagaggtggagtactgcacggccagtgggagctggaatgaccggcagccagtcgattccacgagctataaaattggacaccttgacccagatacagaatatgagattagtgtgctcctgaccaggccaggggagggtggcactggctctcctggtccagctctcaggacaagaacaaagtgtgctgatcccatgcgaggcccaagaaaactagaagtagtggaggtcaaatctcggcaaatcactatccgctgggagccatttggatataatgtaactcgttgccacagttataatctcactgtccactactgttaccaagttggaggacaagaacaagtgcgagaagaagtaagctgggatacagaaaattcacaccctcaacacacgatcactaacctgtcaccatacaccaatgtcagtgtgaaactgatcctcatgaacccagagggccggaaggaaagccaagaactcatagtgcagacagatgaagacctcccaggtgctgttcccactgaatccatacaaggaagtacctttgaagagaagatatttcttcagtggagagaaccaactcaaacatatggtgtaatcactttatatgagatcacctacaaagcagtcagttcctttgacccagaaatagatttatccaatcagagtggaagagtttcaaagctgggaaatgaaacccattttctgttttttggactgtatccggggaccacatactcctttaccatccgagctagcacagctaagggttttgggcctccagcaacaaaccagttcaccaccaaaatatcagcaccctctatgccagcttatgaacttgagacacctttgaatcaaactgacaataccgtgacagtcatgctgaaacctgcccacagcagaggagcacctgtcagtgtctatcaaatagttgttgaggaagaacgtcctcgaagaactaaaaagacgacagaaatcttaaagtgctacccagtgccaattcacttccagaatgcttctctgctgaactcacagtactactttgctgcagaatttcctgcagacagcctccaagctgcgcagccttttacaattggtgataataagacatataatggatactggaacactccccttctcccctataaaagctacagaatttatttccaagctgctagtagagccaatggggaaaccaaaatagactgtgtccaagtggccacaaaaggagctgccactccgaaaccagtcccagaacccgagaaacagacagaccatacagttaaaattgctggagtcatcgcgggcatcttgctgttcgtgattatatttcttggagttgtgttggtaatgaagaaaaggaaactggccaagaagcggaaagagaccatgagcagcacccgacaggagatgactgtgatggtgaactcaatggacaagagctatgctgagcagggcacaaactgcgacgaggctttctcattcatggacacgcacaatctgaatgggagatctgtgtcttcaccatcgtccttcacaatgaaaacaaatacactgagcacatcggtgcctaattcctattacccagatgaaacccacacaatggccagcgataccagcagcctggtgcagtcccatacttacaagaagcgagagccggccgacgtgccctatcagactgggcagctccaccccgccatccgggtggcagacctccttcagcacatcacacagatgaagtgtgcggagggctacggcttcaaggaggaatacgagagcttctttgaagggcagtctgcaccatgggactcggctaagaaagatgagaacagaatgaagaacagatacgggaatatcattgcatacgatcattcccgagtgaggctgcagacaatagaaggagacacaaactcagactatatcaatggcaattatatcgatggttatcatcgacccaatcattacattgctacccaagggccaatgcaggaaaccatctatgacttctggaggatggtgtggcacgaaaacactgcaagtatcatcatggtgaccaatcttgtggaagtgggaagggtcaaatgctgcaaatactggccagatgacacagagatatataaagacattaaagttaccctaatagaaacagaactactggcagaatatgtgataagaacatttgctgttgaaaagagaggtgtgcatgaaatccgagagatcagacagtttcacttcactggctggccggatcatggggtcccctaccatgccaccggcctgctgggattcgtgcggcaagtcaagtccaagagcccgcccagtgcaggcccactggtggtgcactgcagtgctggtgcagggaggactggctgtttcatcgtcattgatatcatgttggacatggccgaaagggaaggggtcgtagacatctacaactgcgtcagggagctgcggtcacggagggtgaacatggtgcaaacagaggagcagtatgtgtttatccacgatgcgatcctggaagcctgtctttgtggggacacctctgtgcctgcttcccaagttaggtctctgtattatgacatgaacaaactggatccacagacaaactcaagccagattaaagaggaattccggacgctaaacatggtgacaccaacgctgcgagtagaggactgcagcatcgcactgttgccccggaaccatgagaaaaaccggtgcatggacatcctgcccccagaccgctgcctgcccttcctcatcaccatcgatggggagagcagcaactacatcaatgctgccctcatggacagctataaacagccttcagcttttatagtcacccagcatcctttgccaaacacagtgaaagacttttggagactggtcctggattatcactgcacatccgtagttatgctaaatgatgtggatcctgcccagttgtgtccacagtactggccagaaaacggagtacacagacacggccccatccaggtggaatttgtctctgctgacctggaagaggacatcatcagcaggatattccgcatttacaatgccgccagaccccaagatggatatcggatggtgcagcaattccagttcctgggctggccgatgtacagggacacaccagtgtctaagcgctccttcttgaagctcattcgccaggtggacaagtggcaagaggaatacaatggcggggaaggcccgaccgttgtgcactgcttgaacgggggaggccgcagtgggacgttctgcgccatcagcatcgtatgtgagatgctccggcaccagagaaccgtggatgtctttcacgctgtgaagacactgaggaacaacaagcccaacatggtcgacctcctggatcagtacaagttctgctacgaggtggccctggaatacttgaattctggctgatggtgtaaacagctctgcaaacaatccctttcataccacaaagccaagacgttccatggtatttgtgcaaaagagatgaagacttctcaatatgcttattttgctttgactaattggctctttttaagagccaagaaagtgtttctaaaattgcttgcactgcccaatcccagtaatgctgctgcctgacagaaacacacacacagccacagttgccaaatncccgtactccttgccacggttctagagcagcgtagacagctggtaaactgaagagcacaactatattcttatgaaggaatttgtacctttggggtattattttgtggcccgtgaccctcgttattgttacagctgagtgtatgtttttgttctgtggagaatgctatctggcattatggtaatatattattttaggtaatatttgtactttaacatgttgcataatatatgcttatgtagctttccaggactaacagataaatgtgtaataacaaagatatgttgtatgagtngtcgtttctgtcagatttgtattgtttccaagggaaaannttgggggaggactcagttcacaaaatgcaaaactcaacgatcagattcacggacccagagcttttccatgtgtttatattgtaa

SEQ ID NO: 104 - DNA, 2178 bp, Homo sapiens
ggccgggtccgggagccccagggcagccgccccgccgagtcgcaggcacagtgtcaccttcgtcccctccggagctgcacgtggcctgagcaggatggtgccctccagcccagcggtggagaagcaggtgcccgtggaacctgggcctgaccccgagctccggtcctggcggcgcctcgtgtgctacctttgcttctacggcttcatggcgcagatacggccaggggagagcttcatcaccccctacctcctggggcccgacaagaacttcacgcgggacgaggtcacgaacgagatcacgccggtgctgtcgtactcctacctggccgtgctggtgcccgtgttcctgctcaccgactacctgcgctacacgccggtgctgctgctgcaggggctcagcttcgtgtcggtgtggctgctgctgctgctgggccactcggtggcgcacatgcagctcatggagctcttctacagcgtcaccatggccgcgcgcatcgcctattcctcctacatcttctctctcgtgcggcccgcgcgctaccagcgtgtggccggctactcgcgcgctgcggtgctgctgggcgtgttcaccagctccgtgctgggccagctgctggtcactgtgggccgagtctccttctccacgctcaactacatctcgctggccttcctcaccttcagcgtggtcctcgccctcttcctgaagcgccccaagcgcagcctcttcttcaaccgcgacgaccgggggcggtgcgaaacctcggcttcggagctggagcgcatgaatcccggcccaggcgggaagctgggacacgccctgcgggtggcctgtggggactcagtgctggcgcggatgctgcgggagctgggggacagcctgcggcggccgcagctgcccctgtggtccctctggtgggtcttcaactcggccggctactacctggtggtctactacgtgcacatcctgtggaacgaggtggaccccaccaccaacagtgcgcgggtctacaacggcgcggcagatgctgcctccacgctgctgggcgccatcacgtccttcgccgcgggcttcgtgaagatccgctgggcgcgctggtccaagctgctcatcgcgggcgtcacggccacgcaggcggggctggtcttccttctggcgcacacgcgccacccgagcagcatctggctgtgctatgcggccttcgtgctgttccgcggctcctaccagttcctcgtgcccatcgccacctttcagattgcatcttctctgtctaaagagctctgtgccctggtcttcggggtcaacacgttctttgccaccatcgtcaagaccatcatcactttcattgtctcggacgtgcggggcctgggcctcccggtccgcaagcagttccagttatactccgtgtacttcctgatcctgtccatcatctacttcttgggggccatgctggatggcctgcggcactgccagcggggccaccacccgcggcagcccccggcccagggcctgaggagtgccgcggaggagaaggcagcacaggcactgagcgtgcaggacaagggcctcggaggcctgcagccagcccagagcccgccgctttccccagaagacagcctgggggctgtggggccagcctccctggagcagagacagagcgacccatacctggcccaggccccggccccgcaggcagctgaattcctgagcccagtgacaaccccttccccctgcactctgtcgtccgcccaagcctcaggccctgaggctgcagatgagacttgtccccagctggctgtccatcctcctggtgtcagcaagctgggtttgcagtgtcttccaagcgacggtgttcagaatgtgaaccagtgactctcgggcgcccctgtggtaactttgcaggcggccctcagtgcatcccacgacccctgcctcgagggccgcctgccttagcaatgggggcctccgcttatcctgctagcaggccccctaggattccccctgccctgtgcgcactctggcggtggccacagcgtgctggcgacactcagggcagctgcctggccatgctgtccctgcactgtgccccgcgggctttgttgctggaagaggtgggtggtgggcttctgcgtccaccaggcctcactggctcatccgcttggggggcttgagacaaatcc

SEQ ID NO: 105 - DNA, 7221 bp, Homo sapiens
cgggcggcctcttgtgtgagggcctgtgggattctccggatatggccggagtgtttccttatcgagggccgggtaacccggtgcctggccctctagccccgctaccggactacatgtcggaggagaagctgcaggagaaagctcgaaaatggcagcaattgcaggccaagcgctatgcagaaaagcggaagtttgggtttgtggatgcccagaaggaagacatgcccccagaacatgtcagggagatcattcgagaccatggagacatgaccaacaggaagttccgccatgacaaaagggtttacttgggtgccctaaagtacatgccccacgcagtcctcaaactcctggagaacatgcctatgccttgggagcagattcgggatgtgcccgtgctgtaccacatcactggagccatttccttcgtcaatgagattccctgggtcattgaacctgtctacatctcccagtgggggtcaatgtggattatgatgcgccgagaaaaaagagataggaggcatttcaagagaatgcgttttcccccttttgatgatgaggagccgcccttggactatgctgacaacatcctaaatgttgagccactggaggccattcagctagagctggaccctgaggaggacgcccctgtgttggactggttctatgaccaccagccgttgagggacagcaggaagtatgtaaatggctccacttaccagcgctggcagttcacactacctatgatgtcaactctctaccgcctggctaatcagctcctgacagacttggtggatgacaactacttctacctgtttgatttgaaggccttctttacgtccaaggcactcaatatggccattcctggaggccccaaatttgaacctcttgttcgagacatcaacctacaggatgaagactggaatgaattcaatgatattaacaagattatcatccggcagcctatccggactgagtacaagattgcttttccttacttgtacaacaatcttccacaccatgtccacctcacctggtaccatactcccaatgttgtattcatcaaaactgaagatcctgacttgccagctttctactttgaccctttgatcaacccaatctcccataggcactcagtcaagagccaggaaccattgccggatgatgatgaggaatttgagctcccggagtttgtggagcccttcctgaaggacacacccctctatacagacaatacagccaatggcattgccctgctctgggccccgcggcccttcaacctacgctctggtcgcacccgtcgggccctggacataccccttgtcaagaactggtatcgggagcattgtcctgccgggcagcctgtgaaagtgagggtctcctaccagaagctgcttaagtactatgtgctgaatgccctgaagcatcggccccctaaggctcaaaagaagaggtatttgttccgctccttcaaagccaccaaattctttcagtccacaaagctggactgggtggagggttggctccaggtttgccgccagggctacaacatgctcaaccttctcattcaccgcaaaaacctcaactacctgcacctggactacaacttcaacctcaagcctgtgaaaacgctcaccaccaaggaaagaaagaaatctcgttttgggaatgctttccacctgtgtcgggaagttctgcgtttgactaagctggtggtggatagtcacgtgcagtatcggctgggcaatgtggatgccttccagctggcagatggattgcagtatatatttgcccatgttgggcagttgacgggcatgtatcgatacaaatacaagctgatgcgacagattcgcgtgtgcaaggacctgaagcatctcatctattatcgtttcaacacaggccctgtagggaagggtcctggctgtggcttctgggctgccggttggcgagtctggctctttttcatgcgtggcattacccctttattagagcgatggcttggcaacctcctggcccggcagtttgaaggtcgacactcaaagggggtggcaaagacagtaacaaagcagcgagtggagtcacattttgaccttgagctgcgggcagctgtgatgcatgatattctggacatgatgcctgaggggatcaagcagaacaaggcccggacaatcctgcagcacctcagtgaagcctggcgctgctggaaagccaacattccctggaaggtccctgggctgccgacgcccatagagaatatgatccttcgatacgtgaaggccaaggctgactggtggaccaacactgcccactacaaccgagaacggatccgccgaggggccactgtggacaagactgtttgtaaaaagaatctgggccgcctcacccggctctatctgaaggcagaacaggagcggcagcacaactacctgaaggacgggccttacatcacagcggaggaaacagtggcagtatataccaccacagtgcattggttggaaagccgcaggttttcacccatcccattccccccactctcctataagcatgacaccaagttgctcatcttggcattggagcggctcaaggaagcttatagtgtgaagtctcggttgaaccagtctcagagggaggagctaggtctgatcgagcaggcctacgataacctccacgaggcgctgtcccgcataaagcgtcacctcctcacacagagagccttcaaagaggtgggcattgagttcatggatctgtatagccacctcgttccagtatatgatgttgagcccctggagaagataactgatgcttacctggaccagtacctgtggtatgaagccgacaagcgccgcctgttcccaccctggattaagcctgcagacacagaaccacctccactgcttgtttacaagtggtgtcaaggcatcaataacctgcaggacgtgtgggagacgagtgaaggcgagtgcaatgtcatgctggaatcccgctttgagaagatgtatgagaagatcgacttgactctgctcaacaggctcgtgcgcctcatcgtggaccacaacatagccgactacatgacagccaagaacaacgtcgtcatcaactataaggacatgaaccatacgaattcatatgggatcatcagaggcctgcagtttgcctcattcatagtgcagtattatggcctggtgatggatttgcttgtattgggattgcaccgggccagtgagatggctgggccccctcagatgccaaatgactttctcagtttccaggacatagccactgaggctgcccaccccatccgtctcttctgcagatacattgatcgcatccatatttttttcaggttcacagcagatgaggctcgggacctgattcaacgttacctgacagagcaccctgaccccaataatgaaaacatcgttggctataataacaagaagtgctggccccgagatgcccgcatgcgcctcatgaaacatgatgttaacttaggccgggcggtattctgggacatcaagaaccgcttgccacggtcagtgactacagttcagtgggagaacagcttcgtgtctgtgtacagtaaggacaaccccaacctgctgttcaacatgtgtggcttcgagtgccgcatcctgcctaagtgccgcaccagctatgaggagttcacccacaaggacggggtctggaacctgcagaatgaggttactaaggagcgcacagctcagtgtttcctgcgtgtggacgatgagtcaatgcagcgcttccacaaccgcgtgcgtcagattctcatggcctctgggtccaccaccttcaccaagattgtgaataagtggaatacagctctcattggccttatgacatactttcgggaggctgtggtgaacacccaagagctcttggacttactggtgaagtgtgagcacaaaatccagacacgtatcaagattggactcaactccaagatgccaagtcggttccccccggttgtgttctacacccctaaggagttgggtggactcggcatgctctcaatgggccatgtgctcatcccccaatccgacctcaggtggtccaaacagacagatgtaggtatcacacactttcgttcaggaatgagccatgaagaagaccagctcattcccaacttgtaccgctacatacagccatgggagagcgagttcattgattctcagcgggtctgggctgagtactcactcaagagacaagaggccattgctcagaacagacgcctgactttagaagacctagaagattcatgggatcgtggcattcctcgaatcaataccctcttccagaaggaccggcacacactggcttatgataagggctggcgtgtcagaactgactttaagcagtatcaggttttgaagcagaatccgttctggtggacacaccagcggcatgatgggaagctctggaacctgaacaactaccgtacagacatgatccaggccctgggcggtgtggaaggcattctggaacacacactctttaagggcacttacttccctacctgggaggggcttttctgggagaaggccagtggctttgaggaatctatgaagtggaagaagctaactaatgctcagcgatcaggactgaaccagattcccaatcgtagattcaccctctggtggtccccgaccattaatcgagccaatgtatatgtaggctttcaggtgcagctagacctgacgggtatcttcatgcacggcaagatccccacgctgaagatctctctcatccagatcttccgagctcacttgtggcagaagatccatgagagcattgttatggacttatgtcaggtgtttgaccaggaacttgatgcactggaaattgagacagtacaaaaggagacaatccatccccgaaagtcatataagatgaactcttcctgtgcagatatcctgctctttgcctcctataagtggaatgtctcccggccctcattgctggctgactccaaggatgtgatggacagcaccaccacccagaaatactggattgacatccagttgcgctggggggactatgattcccacgacattgagcgctacgcccgggccaagttcctggactacaccaccgacaacatgagtatctacccttcgcccacaggtgtactcatcgccattgacctggcctataacttgcacagtgcctatggaaactggttcccaggcagcaagcctctcatacaacaggccatggccaagatcatgaaggcaaaccctgccctgtatgtgttacgtgaacggatccgcaaggggctacagctctattcatctgaacccactgagccttatttgtcttctcagaactatggtgagctcttctccaaccagattatctggtttgtggatgacaccaacgtctacagagtgactattcacaagacctttgaagggaacttgacaaccaagcccatcaacggagccatcttcatcttcaacccacgcacagggcagctgttcctcaagataatccacacgtccgtgtgggcgggacagaagcgtttggggcagttggctaagtggaagacagctgaggaggtggccgccctgatccgatctctgcctgtggaggagcagcccaagcagatcattgtcaccaggaaggacatgctggacccactggaggtgcacttactggacttccccaatattgtcatcaaaggatcggagctccaactccctttccaggcgtgtctcaaggtggaaaaattcggggatctcatccttaaagccactgagccccagatggttctcttcaacctctatgacgactggctcaagactatttcatcttacacggccttctcccgtctcatcctgattctgcgtgccctacatgtgaacaacgatcgggcaaaagtgatcctgaagccagacaagactactattacagaaccacaccacatctggcccactctgactgacgaagaatggatcaaggtcgaggtgcagctcaaggatctgatcttggctgactacggcaagaaaaacaatgtgaacgtggcatcactgacacaatcagaaattcgagacatcatcctgggtatggagatctcggcaccgtcacagcagcggcagcagatcgctgagatcgagaagcagaccaaggaacaatcgcagctgacggcaacacagactcgcactgtcaacaagcatggcgatgagatcatcacctccaccaccagcaactatgagacccagactttctcatccaagactgagtggagggtcagggccatctctgctgccaacctgcacctaaggaccaatcacatctatgtttcatctgacgacatcaaggagactggctacacctacatccttcccaagaatgtgcttaagaagttcatctgcatatctgaccttcgggcccaaattgcaggatacctatatggggtgagcccaccagataacccccaggtgaaggagatccgctgcattgtgatggtgccgcagtggggcactcaccagaccgtgcacctgcctggccagctgccccagcatgagtacctcaaggagatggaacccttaggttggatccacactcagcccaatgagtccccgcagttatcaccccaggatgtcaccacccatgccaagatcatggctgacaacccatcttgggatggcgagaagaccattatcatcacatgcagcttcacgccaggctcctgtacactgacggcctacaagctgacccctagtggctacgaatggggccgccagaacacagacaagggcaacaaccccaagggctacctgccttcacactatgagagggtgcagatgctgctgtcggaccgtttccttggcttcttcatggtccctgcccagtcctcgtggaactacaacttcatgggtgttcggcatgaccccaacatgaaatatgagctacagctggcgaaccccaaagagttctaccacgaggtgcacaggccctctcacttcctcaactttgctctcctgcaggagggggaggtttactctgcggatcgggaggacctgtatgcctgaccgtttccctgcctcctgcttcagcctcccgaggccgaagcctcagcccctccagacaggccgctgacattcagcagtttggcctctttccctctgtctgtgcttgtgttgttgacctcctgatggcttgtcatcctgaataaaatataataataaattttgtataaatagg

SEQ ID NO: 106 - DNA, 223 bp, Homo sapiens
  modified_base (184)..(184): a, c, t, g, unknown or other
tctgatgtcatgcgctttcttctgagggtccgctgctggcagtaccgccagctctctgctctccacagggctccccgccccacccggcctgataaagcgcgccgactgggctacaaggccaagcaaggtacgtgatcgactgcgtggatgcttggataaaattatatccgagaaaagttggaanccagctgttaggaagacactgctgcctcagtgtctctct

SEQ ID NO: 107 - DNA, 1155 bp, Homo sapiens
tgataatcatggcgcccctcggaacaactgtattgctgtggagcctcttgaggagttctccgggcgtggaacgggtctgtttccgggctcgaatccagccctggcacggtggcctgctccaaccgctaccttgctctttcgagatggggctgccacgccgccggttcagctccgaggccgcagaatctggtagcccagagaccaagaaacctacatttatggatgaggaagttcaaagcatactcacgaaaatgacaggcttgaacttgcagaagacttttaagccagctatacaagaactgaagccaccaacctataagctaatgactcaggcacagttggaagaggctacaagacaggcagttgaggcagctaaagtacgattaaaaatgccaccagttctggaagagcgagtaccaataaatgatgtgttagctgaagataagattttggaaggaacagaaacaaccaaatatgtgtttactgatatatcatatagcataccacaccgggagcgttttattgtcgtcagagaaccaagtggcacactacgcaaagcctcttgggaagaacgggaccgaatgatacaagtttatttcccaaaagaaggtcgtaaaattttgacaccaataattttcaaggaagaaaatcttaggactatgtatagccaggacaggcatgttgatgtcctcaatctctgctttgcccagtttgagccagattccacagagtatatcaaggttcatcacaagacctatgaagatatagataaacgtggaaaatatgaccttttacgttcaacaagatactttggtggaatggtgtggtattttgtaaataataaaaagattgatggtttgctgattgaccagattcagagagatctaatcgatgatgcaaccaacttggtccagctgtatcacgtgctccatccagatggccagtcggctcaaggggccaaggatcaggctgctgagggaataaatttaatcaaggtctttgcaaaaacagaagcacagaagggagcctatatagaactaacactgcagacttatcaagaagcactcagtcgccattctgcagcttcctaaaaatattttaaaaatacatttattttactaaaaaaaaaaaaaaaaaaaaaaaaaaaaaaaaaaa

SEQ ID NO: 108 - DNA, 6550 bp, Homo sapiens
aataaagaacgaggacactgcggggtgtgcggcggcccaagcggtttcaaacggcttagagcaggccgcttggttctgacccagctgaggaaatactcttaattctaaggaaaacctggaagcacaatgggagatgacagtgagtggttgaaactgccagttgatcagaaatgtgaacacaagctgtggaaagcaaggttaagtgggtatgaagaggccctgaagatcttccagaaaataaaggatgaaaagagcccagagtggtccaaatttttaggattgatcaaaaaatttgtcactgattccaatgcagtggttcaattgaaaggattagaagctgcacttgtttatgttgaaaatgcccatgtagcaggaaaaaccacaggagaagttgtgtcaggtgttgtaagtaaggtgttcaatcaacctaaagctaaagccaaggagctgggcatagagatctgtcttatgtacatagagattgagaaaggagaggctgttcaagaagagctcctgaaaggcttggacaataagaatcccaagatcatagtggcctgtatagagacactgaggaaagccttaagtgaatttggttccaaaatcatcttgcttaagccaattatcaaagtgttgccaaaactctttgagtctcgagagaaggctgttcgagatgaagccaaactaattgctgtggagatttacagatggattcgggatgctctgagacccccattacaaaatataaactctgttcagttgaaagaactagaagaagaatgggtcaaactgccaacaagtgctcctagacctactcgatttcttcgttcccaacaagaactagaagctaaattggaacaacaacagtctgctggtggagatgctgaaggaggtggtgatgatggtgatgaggtgccacaaatagatgcttatgagcttttagaagctgtagaaatcctttccaaacttcccaaagacttttatgacaaaattgaggcaaaaaaatggcaagagagaaaagaggccctggagtctgtagaagtactaataaaaaaccccaaactggaagctggcgattatgcagatttagtaaaagcattaaagaaggttgttggaaaggacaccaatgtcatgttggtggctttggcagcaaaatgtcttactggcctggctgttgggctaaggaagaaatttggacaatatgcaggacatgttgtgccaaccatcttggagaaattcaaagagaagaaacctcaagtggtacaagccctgcaggaggcaattgatgcaatcttccttactaccacactacagaacatcagtgaggatgttttagcagtaatggataataaaaatccaaccatcaagcagcagacatctctttttattgcaagaagtttccgccactgcactgcttctaccctgccaaagagcttgctaaagcccttttgtgctgcactacttaagcacatcaatgattctgctcctgaagtcagagatgccgcatttgaagcattgggtactgctttgaaggtggttggcgagaaagcagtaaacccattcctagctgatgtggacaaactcaagcttgataagatcaaagaatgttcagaaaaggtagaactgatacatggtaagaaagctggactagctgctgataagaaggaattcaaacctctgcctggaaggactgctgcttcaggggctgcaggagataaggacacaaaggacatttctgcacccaaaccaggacctctaaaaaaggcacctgctgctaaggctggtgggccaccaaaaaaggggaaaccagctgcaccaggaggcgcagggaatactggaaccaagaacaagaaaggactggagactaaagaaatagtggagcctgagctctcgatagaagtatgtgaagaaaaagcttcagctgttcttccccctacctgtatacagcttcttgacagcagtaactggaaagaaaggctggcttgtatggaagagttccagaaggctgttgagctaatggaccgaactgaaatgccatgccaggcattagtgaggatgctagccaagaaacctggatggaaagaaactaattttcaggtgatgcaaatgaagcttcatatagttgctttgattgcccagaagggaaatttttccaaaacgtcagctcaggttgtattagatggccttgtggacaagattggagatgtgaaatgtgggaacaatgcaaaagaagctatgacagcaatagccgaagcctgtatgttaccatggactgctgaacaggttgtgtcaatggctttctcacaaaagaatcccaaaaatcagtcagaaactctgaattggctatcaaatgccataaaagaatttggtttttctgggttgaatgtcaaagctttcattagcaatgtgaagacagctcttgctgcaacaaacccagctgtgaggactgctgccataaccctgcttggcgtgatgtatctgtatgttggtccctctttgcgaatgttctttgaggatgagaagcctgccctcctatcccagatagatgcagaatttgagaagatgcagggacaaagtccacctgctccaaccagaggaatttccaagcatagcacaagtggtacagatgaaggagaagatggagatgaaccagatgacgggagcaatgatgtcgttgatcttttgccgaggacggagatcagtgataaaatcacttcagagttggtatctaagattggtgataagaattggaagattaggaaagaaggcctagatgaagtggcaggtattattaatgacgcaaaatttatccaaccgaatataggtgaacttccaactgccttgaagggtcgactcaatgattcaaataaaatcttggtacagcaaacgctgaatatcctgcaacaactggcagtagccatgggcccaaatattaagcaacatgtaaaaaatttaggcatccctatcatcacagtccttggagacagcaagaacaatgttcgagctgctgccctagcgactgtgaatgcttgggcagaacagactggcatgaaggaatggctggaaggagaagatctttctgaagagctcaaaaaggaaaatcctttcttgaggcaagagcttctgggctggctggctgagaaactacctactcttcgttccacccctacagaccttatcctttgtgttcctcatctctactcctgcctagaagatcgaaatggagatgtgcgaaagaaggcccaagatgccttgccattcttcatgatgcatttaggatatgaaaaaatggccaaggctactgggaaactaaagccaacttctaaagatcaggtattggccatgctagagaaagccaaagttaacatgccagccaagcctgctccacccactaaagcaacttctaaaccaatgggagggtccgctccagccaaattccagcctgcatcagcacctgctgaagattgtatttccagcagtacagaacccaaacctgatccaaaaaaggccaaagctccaggattatcctctaaagcaaagagtgcacaagggaagaagatgccaagcaaaaccagcttaaaggaggatgaagacaaatccgggcctatttttattgttgttccaaatggaaaagagcaaaggatgaaagatgaaaaaggattgaaggtgctaaagtggaattttactaccccacgggatgaatacattgagcaactaaagactcaaatgtctagctgtgtggctaaatggttacaagatgagatgtttcactcagactttcagcatcataacaaagcccttgctgttatggttgatcacttggagagtgaaaaagaaggagttattggttgcctggatcttatcttaaagtggcttaccctgaggttttttgacaccaatacaagcgtcctgatgaaagcactagaatatttaaaattgctcttcaccttgctaagtgaagaagaatatcatcttactgagaatgaagcatcttccttcatcccctatcttgtcgtcaaggttggagaaccaaaggatgtcattcgtaaagatgttcgtgccatcctgaaccggatgtgccttgtctacccagctagcaagatgtttccctttatcatggaaggaaccaaatccaaaaactctaagcagagagcagagtgcctggaagagctgggatgtctggttgagtcctatggcatgaatgtttgccaaccaaccccaggaaaagccttaaaggaaatagctgttcacataggagaccgtgacaatgctgtacgcaatgctgcactcaacaccattgtaacggtgtacaatgtacatggggatcaggtgttcaaactgattggaaatctttctgaaaaggatatgagcatgctcgaggagaggattaagcggtcagcaaagagaccctctgctgcaccaataaaacaggtggaagagaaacctcagcgtgcacagaacataagctccaatgccaacatgttacgcaagggaccagctgaggacatgtcttccaaactcaaccaagcccgaagcatgagtgggcatcctgaggcagcccagatggtccgccgagaattccagctggatctagatgagattgagaatgacaatggtacagtccgatgtgaaatgccagaacttgttcagcacaaactggatgacatttttgagccagtccttattcctgaacccaagatccgggctgtttctccacacttcgatgacatgcacagtaatacagcatccacaatcaatttcattatctcccaagtagccagtggtgacatcaacacaagtatccaagctctgacacagctgtttcagatagagagccttgcccgggaggcctccactggagtactaaaagacctaatgcatggcctcatcaccttaatgctggattctcggattgaagatcttgaggaaggacaacaggtcatccgctctgtgaacctcttggtggtgaaggttctggagaagtcagaccagaccaacatcctgagtgccctacttgttttgctccaagacagcctgctagcaacagccagttctcccaaattctcagagcttgttatgaagtgtctctggagaatggttcgactgttgcctgataccatcaatagcattaacctagacagaattcttctggatatccacattttcatgaaggtcttccccaaagagaaactgaagcaatgcaaaagtgaatttcccataaggaccctaaagaccctgctacacaccttatgcaaattaaaagggcccaagatcctggaccacctaacgatgatcgacaacaaaaacgagtctgagctggaggcccatctctgccggatgatgaagcacagtatggaccagactgggagcaagtctgataaggaaacagaaaagggagcatctcgaatagatgaaaaatcatcaaaggccaaagtgaatgatttcttagctgagatttttaagaagattggctctaaagaaaacactaaagagggactagcagagttatatgaatataagaagaaatactcagatgctgacattgaaccatttctgaaaaattcctcacagttcttccagagctatgtcgaaagaggccttcgggtgattgagatggagagggagggcaaaggtcgtatttccacttcaacaggcatctcccctcagatggaagtcacatgtgtgcccacgcccacaagcacagtgtcctccataggtaacacaaatggggaagaagtggggccatctgtctacttggaaaggctaaagatcctccgacagcgatgtggtctggacaacacaaagcaagatgaccgacctcctttgacctctttgctctccaaaccagcagttcctactgtcgcctcttccacagacatgctccacagcaaactctctcagctccgggagtcacgggagcagcaccagcattcagacctggattctaaccagactcactcttcaggaactgtgacctcctcctcctccacagctaacatagacgacttgaaaaaaagactggagagaataaagagcagtcgcaaatgaagctgccccactcccccggcaccctgcagctttagtttactaaactagaagtcctcatagtttaaaatggcctcagcaggcctagtgtatacaaactggttgtatgtatcatgccgtggagctagggggaggagtcattgtggcacaagtatttgtacatactctgcttctctctgtcagcgtcctgctgctctagaagactgtccgtggatgagtttagtgtacagacttgtaaacagctgccccctctctgctcagtctagttcccagatccttttcttttctttttaattgctcatttgtaaaattgtcctaatctttcctagctttttaatagttaatattagaaactctttaatagttttcctttcagtttgtgagctcttctctgtcgccctgaagggtcactgtattctgtatgaatgcatggcatgatacaactaatttaagagtcttttataaataaagtttgcattaactatacctgacaaaaaaaaaaaaaaaa

SEQ ID NO: 109 - DNA, 2369 bp, Homo sapiens
gggaccagatggattgtagggagtagggtacaatacagtctgttctcctccagctccttctttctgcaacatggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgagatgccttcagttcctgaacacggaaggaaagacgcagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagagatcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccagacatccttgaaatacaatattctcccagaaaaggaagagttcccctttgctttaggagtgcagactctgcctcaagcttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctgaaaccagccatagtgaaagtctatgattactacgagacggatgagtttgcaattgctgagtacaatgctccttgcagcaaagatcttggaaatgcttgaagaccacaaggctgaaaagtgctttgctggagtcctgttctcagagctccacagaagacacgtgtttttgtatctttaaagacttgatgaataaacactttttctggtcaaaaaaaaaaaagaaaaaaaaaaagaaaaaaaaaaaaaaaaaaaa

SEQ ID NO: 110 - DNA, 1714 bp, Homo sapiens
ggggcggcggcggcggtagcggtggccttggttgtcttccagtctcctcggctcgccctttagccggcaccgctccccttccctcccccttcctctcttccttccttccctccccttccctttttcccttccccgtcggtgagcggcgggggtggctccagcaacggctgggcccaagctgtgtagaggccttaaccaacgataacggcggcgacggcgaaacctcggagctcgcagggcgggggcaaggcccgggccttggagatggagaattctcagttgtgtaagctgttcatcggcggcctcaatgtgcagacgagtgagtcgggcctgcgcggccactttgaggcctttgggactctgacggactgcgtggtggtggtgaatccccagaccaagcgctcccgttgctttggcttcgtgacctactccaatgtggaggaggcggacgccgccatggccgcctcgccccatgccgtggacggcaacactgtggagctgaagcgggcggtgtcccgggaggattcggcgcggcccggtgcccacgccaaggttaagaagctctttgtcggaggccttaaaggagacgtggctgagggcgacctgatcgagcacttctcgcagtttggcaccgtggaaaaggccgagattattgccgacaagcagtccggcaagaagcgtggattcggcttcgtgtatttccagaatcacgacgcggcagacaaggccgcggtggtcaagttccatccgattcagggccatcgcgtggaggtgaagaaagcagtccccaaggaggatatctactccggtgggggtggaggcggctcccgatcctcccggggcggccgaggcggccgggggcgcggcggtggtcgagaccagaacggcctttccaagggcggcggcggcggttacaacagctacggtggttacggcggcggcggaggcggcggctacaatgcctacggaggcggcggcggcggttcgtcctacggtgggagcgactacggtaacggcttcggcggcttcggcagctacagccagcatcagtcctcctatgggcccatgaagagcggcggcggcggcggcggtggaggcagtagctggggcggtcgcagtaatagtggaccttacagaggcggctatggcggtgggggtggctatggaggcagctccttctaaaagaaaatttaaaatgcctgggagtggctataggggtagctctttccaacagcccaagtggggtcaactcctaagccccaccccctcacacacaccgccttccctgttttgcccttgggggagccacttctaaggctgcttacccttgggggtgttcctctatttgcctgccacctctcttgtctctccctctgaagatggactcggccccacatacacatttttgtgttacagtcattgatggactctatttttttattattacttggaccttggtcgtttttatactagcaaaatgtcttgttttaatttgtgttttttggggggagggagggagtgaacttgctgattctgtagcaaaacctgggtgggggttggggtggggggtagtttactttgttgtaaggacttgataacctggctacagcgttttctatgaaatctacttggatcccatgcctgaaatttggaagcatatgtacacaaatcatttttacgttttatttttaataaatcattgtgtttgaccgta

SEQ ID NO: 111 - DNA, 322 bp, Homo sapiens
gagcctagtgaatggcaagcccatgcggggtgggtttgggtatgaggaggaggtcattgctggctggagtggccaagaggggtttcacaaagaagagtagcatctaaactggattgacaggaatggattggaagtgagacatttcagtgggaaaagggaattgtgccgtctgtgtcattttgtcctttttgtgctgttttttgtgtcctaaccagctgcttttctacagctactctcctctgcaaggcctaccgtgggggccacttaaccatccgccttgccctgggtggctgcaccaatcggccgttctaccgcattgtgg

SEQ ID NO: 112 - DNA, 1964 bp, Homo sapiens
cttcttccgacagcttgctgccctagaccagagttggtggctggacctcctgcgacttccgagttgcgatgctgtacttctctttgttttgggcggctcggcctctgcagagatgtgggcagctggtcaggatggccattcgggctcagcacagcaacgcagcccagactcagactggggaagcaaacaggggctggacaggccaggagagcctgtcggacagtgatcctgagatgtgggagttgctgcagagggagaaggacaggcagtgtcgtggcctggagctcattgcctcagagaacttctgcagccgagctgcgctggaggccctggggtcctgtctgaacaacaagtactcggagggttatcctggcaagagatactatgggggagcagaggtggtggatgaaattgagctgctgtgccagcgccgggccttggaagcctttgacctggatcctgcacagtggggagtcaatgtccagccctactccgggtccccagccaacctggccgtctacacagcccttctgcaacctcacgaccggatcatggggctggacctgcccgatgggggccatctcacccacggctacatgtctgacgtcaagcggatatcagccacgtccatcttcttcgagtctatgccctataagctcaaccccaaaactggcctcattgactacaaccagctggcactgactgctcgacttttccggccacggctcatcatagctggcaccagcgcctatgctcgcctcattgactacgcccgcatgagagaggtgtgtgatgaagtcaaagcacacctgctggcagacatggcccacatcagtggcctggtggctgccaaggtgattccctcgcctttcaagcacgcggacatcgtcaccaccactactcacaagactcttcgaggggccaggtcagggctcatcttctaccggaaaggggtgaaggctgtggaccccaagactggccgggagatcccttacacatttgaggaccgaatcaactttgccgtgttcccatccctgcaggggggcccccacaatcatgccattgctgcagtagctgtggccctaaagcaggcctgcacccccatgttccgggagtactccctgcaggttctgaagaatgctcgggccatggcagatgccctgctagagcgaggctactcactggtatcaggtggtactgacaaccacctggtgctggtggacctgcggcccaagggcctggatggagctcgggctgagcgggtgctagagcttgtatccatcactgccaacaagaacacctgtcctggagaccgaagtgccatcacaccgggcggcctgcggcttggggccccagccttaacttctcgacagttccatgaggatgacttccggagagttgtggactttatagatgaaggggtcaacattggcttagaggtgaagagcaagactgccaagctccaggatttcaaatccttcctgcttaaggactcagaaacaagtcagcgtctggccaacctcaggcaacgggtggagcagtttgccagggccttccccatgcctggttttgatgagcattgaaggcacctgggaaatgaggcccacagactcaaagttactctccttccccctacctgggccagtgaaatagaaagcctttctattttttggtgcgggagggaagacctctcacttagggcaagagccaggtatagtctcccttcccagaatttgtaactgagaagatcttttctttttcctttttttggtaacaagacttagaaggagggcccaggcactttctgtttgaacccctgtcatgatcacagtgtcagagacgcgtcctctttcttggggaagttgaggagtgcccttcagagccagtagcaggcaggggtgggtaggcaccctccttcctgtttttatctaataaaatgctaacctgcaaaaaaaaaaaaaaaa

SEQ ID NO: 113 - DNA, 2769 bp, Homo sapiens
ctcccagccccctctgtttagatcagggaatttcagacatgcacactcgggtagggaatcttatgaacagaaccaggacagggaggctggccggaggttcctgcagagggagcgtcaaggccctgtgctgctgtccctgggggccagaggggttgcccagcatgcccactggcaggagagagggaactgacccacttgctcctaccagcttctgaaggctccaaagtccggaggtgcagaaagccaggaccaagagacaggcagctcaccagggtggacaaatcgccagagatgtggtgcattgtcctgttttcacttttggcatgggtttatgctgagcctaccatgtatggggagatcctgtcccctaactatcctcaggcatatcccagtgaggtagagaaatcttgggacatagaagttcctgaagggtatgggattcacctctacttcacccatctggacattgagctgtcagagaactgtgcgtatgactcagtgcagataatctcaggagacactgaagaagggaggctctgtggacagaggagcagtaacaatccccactctccaattgtggaagagttccaagtcccatacaacaaactccaggtgatctttaagtcagacttttccaatgaagagcgttttacggggtttgctgcatactatgttgccacagacataaatgaatgcacagattttgtagatgtcccttgtagccacttctgcaacaatttcattggtggttacttctgctcctgccccccggaatatttcctccatgatgacatgaagaattgcggagttaattgcagtggggatgtattcactgcactgattggggagattgcaagtcccaattatcccaaaccatatccagagaactcaaggtgtgaataccagatccggttggagaaagggttccaagtggtggtgaccttgcggagagaagattttgatgtggaagcagctgactcagcgggaaactgccttgacagtttagtttttgttgcaggagatcggcaatttggtccttactgtggtcatggattccctgggcctctaaatattgaaaccaagagtaatgctcttgatatcatcttccaaactgatctaacagggcaaaaaaagggctggaaacttcgctatcatggagatccaatgccctgccctaaggaagacactcccaattctgtttgggagcctgcgaaggcaaaatatgtctttagagatgtggtgcagataacctgtctggatgggtttgaagttgtggagggacgtgttggtgcaacatctttctattcgacttgtcaaagcaatggaaagtggagtaattccaaactgaaatgtcaacctgtggactgtggcattcctgaatccattgagaatggtaaagttgaagacccagagagcactttgtttggttctgtcatccgctacacttgtgaggagccatattactacatggaaaatggaggaggtggggagtatcactgtgctggtaacgggagctgggtgaatgaggtgctgggcccggagctgccgaaatgtgttccagtctgtggagtccccagagaaccctttgaagaaaaacagaggataattggaggatccgatgcagatattaaaaacttcccctggcaagtcttctttgacaacccatgggctggtggagcgctcattaatgagtactgggtgctgacggctgctcatgttgtggagggaaacagggagccaacaatgtatgttgggtccacctcagtgcagacctcacggctggcaaaatccaagatgctcactcctgagcatgtgtttattcatccgggatggaagctgctggaagtcccagaaggacgaaccaattttgataatgacattgcactggtgcggctgaaagacccagtgaaaatgggacccaccgtctctcccatctgcctaccaggcacctcttccgactacaacctcatggatggggacctgggactgatctcaggctggggccgaacagagaagagagatcgtgctgttcgcctcaaggcggcaaggttacctgtagctcctttaagaaaatgcaaagaagtgaaagtggagaaacccacagcagatgcagaggcctatgttttcactcctaacatgatctgtgctggaggagagaagggcatggatagctgtaaaggggacagtggtggggcctttgctgtacaggatcccaatgacaagaccaaattctacgcagctggcctggtgtcctgggggccccagtgtgggacctatgggctctacacacgggtaaagaactatgttgactggataatgaagactatgcaggaaaatagcaccccccgtgaggactaatccagatacatcccaccagcctctccaagggtggtgaccaatgcattaccttctgttccttatgatattctcattatttcatcatgactgaaagaagacacgagcgaatgatttaaatagaacttgattgttgagacgccttgctagaggtagagtttgatcatagaattgtgctggtcatacatttgtggtctgactccttggggtcctttccccggagtacctattgtagataacactatgggtggggcactcctttcttgcactattccacagggataccttaattctttgtttcctctttacctgttcaaaattccatttacttgatcattctcagtatccactgtctatgtacaataaaggatgtttataagcaaaaaaaaaaaaaaaaaaaaaaaaaaaaaaaaa

SEQ ID NO: 114 - DNA, 430 bp, Homo sapiens
cttctcttaccgccatcttggctcctgtggaggcctgctggaacggacttctaaaaggaactatgtctggaaggctgtggtccaaggccatttttgctggctataagcggggtctccggaaccaaagggagcacacagctcttcttaaaattgaaggtgtttacgcccgagatgaaacagaattctatttgggcaagagatgcgcttatgtatataaagcaaagaacaacacagtcactcctggcggcaaaccaaacaaaaccagagtcatctggggaaaagtaactcgggcccatggaaacagtggcatggttcttgccaaattccgaagcaatcttcctgctaaggccattggacacagaatccgagtgatgctgtacccctcaaggatttaaactaacgaaaaatcaataaataattgtggatttgt

SEQ ID NO: 115 - DNA, 369 bp, Homo sapiens
ggggtcccgaggtcgcagcttccactggcgcgggttgagttccctgttgcccttggtctcggggtcgctgtaggcgctgaggctgcagctatcatggtgaacttacttcagattgtgcgggaccactgggttcatgttcttgtccctatgggatttgtcattggatgttatttagacagaaagagtgatgaacggctaactgccttccggaacaagagtatgttatttaaaagggaattgcaacccagtgaagaagttacctggaagtaaagactggctagattatcgaatgttcacattttaaagttctgagagaaataaaaacatgaagaatctgagaaaaaaaaaaaaaaaaaaaaaaaaaagaaa

SEQ ID NO: 116 - DNA, 447 bp, Homo sapiens
ggcggtggcgcgagttggactgtgaagaaacatggcggccgcgacgttgacttcgaaattgtactccctgctgttccgcaggacctccaccttcgccctcaccatcatcgtgggcgtcatgttcttcgagcgcgccttcgatcaaggcgcggacgctatctacgaccacatcaacgaggggaagctgtggaaacacatcaagcacaagtatgagaacaagtagttccttggaggcccccatccaggccagaaggaccaggtccacccagcagctgtttgcccagagctggagcctcagcttgaagatgatgctcaaggtactcttcatggaccaccattcgctgttggcaagaaacggctttacttacaaaacagactctttaccttctgctgtgtttgaagtatgtttagtcagcatgctcaggaaataaatgtgaattgcccttg

SEQ ID NO: 117 - DNA, 468 bp, Homo sapiens
caagatggcggggagcggcgtccgccaagctacttctaccgccagcaccttcgtgaagcccattttcagtcgggacatgaacgaggccaagcggagggtgcgcgagctctaccgcgcctggtatcgggaggtgccgaacactgtgcaccaattccagctggacatcactgtgaaaatgggacgggataaagtccgagaaatgtttatgaagaatgcccatgtcacagaccccagggtggttgatcttctggtcattaagggaaagatcgaactggaagaaacaattaaagtatggaagcagcggacacatgttatgcggttcttccatgaaacagaagcgccaaggccaaaggatttcctatccaagttctatgttggccacgatccatgaagtcattcagtggaaagatgcacgttgatactattttagagcacaaataaactcactatacaatgaaaaaaaaaaaa

SEQ ID NO: 118 - DNA, 5922 bp, Mus musculus
gaggggctggatttggactgggtaaccaggtactctgagcaaatggcgagaccagaggacgaattatgacactccttcgaaagaatgaattaaacgtgcctccgcatgtgtctgattgacaggcctcctggggaaagggcgtataaaggagccggtcactgccgggaggcagagagagccagtccgatgtctgcagcctccctggccaggcctctcctctcctgccgcagctagtccccctcaggacagacagagtactggcgtcggtcaccattcacttgcaaacacaccaggtcacgtgaagaaacttcctggtgacactcaggctgtagctgtgcactcttcaaccacgaggttggttttctcctaagtgtcacaggtggagacaagatgctctgggcactggccctgctggctctgggcatagggccaagagcttctgctggtgaccacgtcaaggacacttcatttgaccttttcagcatcagcaacattaaccggaagaccatcggtgccaagcagttccgagggcctgaccccggggtgcccgcctaccgttttgtacggtttgactacatccccccagtgaacacagatgatctcaacaggattgtcaagcttgcaaggagaaaggagggcttcttcctcacagcccaactgaagcaggaccgcaagtctcggggaacgctcctggtgttggaaggccccggcacctcccagaggcagtttgagattgtgtccaatggcccaggggacactttggacctcaactactgggtagaaggcaatcagcataccaacttcctggaggatgtgggcctggctgactcccagtggaagaatgtgactgtgcaggtggccagtgacacctatagcctgtatgtgggctgcgatcttatcgacagtgtcaccctggaagaaccattctatgagcagctagaagtagacaggagcaggatgtacgtggccaaaggtgcatctcgagagagtcacttcaggggcttgctgcagaatgtccatctcgtgtttgcagattctgtggaagatatcttaagcaagaaaggctgtcaacacagccagggagctgaagtcaacaccatcagtgaacatacagagactctccatctgagccctcacatcaccacagatctcgtggtccagggtgtggagaaggcacaggaggtgtgtacgcactcctgcgaggagttgagcaacatgatgaatgagctctctggactgcacgtcatggtgaaccagctgagcaagaacctggagagagtgtctagtgataaccagttccttttggagctcattgggggccctctgaagacaagaaacatgtcagcctgtgtgcaggagggccgaatctttgcagaaaatgaaacctgggttgtggatagttgtaccacatgcacctgcaagaaatttaaaacagtctgccatcagatcacctgctcacctgcaacttgtgccaacccatcttttgtggaaggcgagtgctgtccatcctgttcacactctgcagacagtgatgagggctggtctccgtgggcagagtggaccgagtgttctgtcacctgtggctctgggacccagcagagaggccggtcttgtgatgtcaccagcaacacctgcctgggcccctccattcagacaaggacatgcagcctgggcaaatgtgatacgagaatccgtcagaatggaggctggagtcactggtcaccctggtcttcatgctccgtgacttgtggagttggcaatgtcacccgcatacgtctctgcaactcaccagtgccccagatgggtggcaagaactgcaagggcagcggccgggaaaccaaaccctgtcagcgtgatccgtgcccaattgatggccgctggagcccctggtccccttggtcagcctgcacagttacctgtgctggagggatccgtgagcgctcacgtgtttgcaacagccctgagccccagtatggagggaaggactgtgtcggggatgtgacagaacaccaaatgtgcaacaagagaagctgccctattgatgggtgcttatccaacccgtgttttcctggagccaagtgcaacagcttccctgatgggtcctggtcctgtggctcctgcccagtgggctttctgggcaatggtacccactgtgaggacctggatgagtgtgctgtggtcacagatatttgcttctcaactaacaaagctccccgctgtgtcaacaccaacccgggcttccactgcctgccttgtccaccacgctacaaggggaaccaacccttcggtgttggcctggaggatgctaggacagaaaaacaagtgtgtgagccagagaatccatgtaaggacaagactcacagctgccacaagaatgcagagtgcatctacctgggccactttagtgaccccatgtacaagtgtgagtgccagattggctacgcaggtgatgggctcatctgcggggaggactcagacctggatggctggcccaacaacaacctggtgtgtgctactaatgccacctaccactgcatcaaggacaactgccccaaactgccaaattccgggcaggaggattttgataaggatggaatcggagatgcttgtgacgaggacgatgacaatgacggtgtgagcgatgagaaggacaattgccagcttctcttcaatccccgtcaattagactatgacaaggatgaggttggagaccgctgtgacaactgcccctatgtgcacaacccagcacagatcgacacagacaacaatggcgagggggatgcctgctctgtggacattgacggagacgatgttttcaatgagcgagacaattgtccatatgtctacaacactgaccagagagatacggatggtgatggcgtgggtgaccactgtgacaattgtcctctgatgcacaacccagatcagatcgatcaggacaatgatctcgttggagaccagtgtgacaacaatgaggacatagatgatgacggccaccagaacaaccaagacaactgcccatacatctccaactccaaccaggctgaccatgacaacgacggcaagggcgatgcctgcgactctgatgatgacaatgatggtgttccagatgacagggacaactgtcggcttgtgttcaacccagaccaggaagactcggacggtgacggccgaggtgacatttgtaaagatgactttgacaatgataatgtcccagatattgatgatgtgtgccctgagaacaatgccatcactgagacagacttcagaaacttccagatggtccctctggatcccaaggggaccacacaaattgatcccaactgggtaattcgtcaccaaggcaaagagctggtgcagacagcaaactcagaccctggcatcgctgtaggtttcgacgagtttgggtctgtggacttcagtggcactttctatgtcaacactgaccgggatgatgactacgctggctttgtctttggctatcagtcaagcagccgcttctatgtggtgatgtggaagcaggtgacccagacctactgggaagacaagcccagtcgggcttacggctactctggtgtgtcactcaaagtggtaaactccacgactggtactggcgagcacctgaggaatgccctgtggcacacgggaaacacagaaggccaggtccggactctatggcatgaccccaaaaacattggctggaaagactacactgcctacaggtggcacctgattcacaggcctaagacaggctacatgagagtcttagtgcatgaaggaaagcaagtcatggctgactcaggaccaatttatgaccaaacctacgctggtggacggctgggcctgtttgtcttctcccaagagatggtctatttctcggacctcaagtatgagtgcagagatgcctagagagcagggctccagctccagcaatgtgctgcaaacaccccttcttagacacatcagtccatcttggcacttgtggcttttctgtcatttggcatttcctgtttcttgaccttaactgagtggatctacacctccttcatcagcaccaagtccaagtgtcttcaaaggagaaacatcaattgcactccaagagcttccagcctgctgctggaaaacatttggatgagatatgaggctcaccgtggagcgaagaccgagcattccgctgtgttgccttttcttgtttgtttaaaaagaatgacgtttacatgtaaatgtaattacttgcagtatttatgtgtatatggagtcgaagggagtattgtgtttaagtcattctcataaattaagcatgaaaaatatggctcacttaccctcagtgcttaatgttgtcactgtttttggattcgtgctgttctataatgacacacaaatcctgtgaggctgaacaggaagaaagatcaggtgaactcggctgcagtaaggaagaggccagaagacagtcatgtgagactgtggccccttgtccagtgatttgaaacattaactaattagaacataggaagtgatcgaaaagttagaagaaacgcagttcttacttggccacagttaaagctgactcgacagagacaaagaagctgcatcctttgaaaggggaatatttcagggttccagctgtaggcttctcagagctttcagaagaaaacctcagcagaggcaatgtccatttcagctgcctgcctgggttgggcagtgtggaaagcatacctggctggacgtcatcaggctctgcatactctgcatactgactgctaggatctttgtctgggcatcctttcctgtccctgctgctaggaaacagactggcaaataagttggcagatgcactttggtaccctgttcctactctaggctttagaatgatgcaaaagctgcttttgccaaatttggtaatgcatttccttaaggttccaggtacggctcttttgttaatatttattaaatgatttggaatatagcttcactcatcaataatttattttaaacatacaaagtaacaagtatgtagtatataatttctagaaataagcatatgacaaggatataatgatataaagacacacttacaaatatctgctgtttgaaatacaaggatttatgtcatgatagaatggaaccttaatatgcatccttaccaataattttaattcagttctttttactattatctctttgttgtatataggttctttttattttaatgcttaaggaaacatcatttgttttttccagacacacccattaatcagtcacatcattacagacatatgcttgatttattttattttgtttttgttttgctttaagctgcatgatctcccagcaggaagtactgcctctctcacttcacacgagctcagtggaagaatattttgtccctctggtggggtagttcacagtcccctttctagtaatggcaactgcatttctaagttagacagaaagaatccacgattttgatctgagtttccacaatttgtctagatggagttatttgtgttgaaatctctgagggtgagtttccttgtttcttgttatcctgcactttttagatttttgtggaactgtattcccaaggccagcacaggtcaggagacttctggccatgagggtgtgaagaacatttaggctctcacttgggcatttggttcaatggtacaaggtggtgggagcgatgtgaatacattcaatgtaccatattatttttgtaaattatttatgtagttttttttaaaaacagaagcttctcatatacgttaatgaaacattgtttttttttttttgccaaagattgtaaatatttatttatttgttcattgggttaaatttcaccactaaaacccctcatttagctagagcttcatttttataccaaagattatcaacagcaaataaattataaaaaggtttctg

SEQ ID NO: 119 - DNA, 518 bp, Homo sapiens
ccgtagtgtctcattgcagataatttttagcttagggcctggtggctaggtcggttctctcctttccagtcggagacctctgccgcaaacatgctccgccagatcatcggtcaggccaagaagcatccaagcttgatccccctctttgtatttattggaactggagctactggagcaacactgtatctcttgcgtctggcattgttcaatccagatgtttgttgggacagaaataacccagagccctggaacaaactgggtcccaatgatcaatacaagttctactcagtgaatgtggattacagcaagctgaagaaggaacgtccagatttctaaatgaaatgtttcactataacgctgctttagaatgaaggtcttccagaagccacatccgcacaattttccacttaaccaggaaatatttctcctctaaatgcatgaaatcatgttggagatctctattgtaatctctattggagattacaatgattaaatcaataaataactgaaaaaaaaaa

SEQ ID NO: 120 - DNA, 4595 bp, Homo sapiens
gcatgctcgacgcgccatgtgatggatatctgcagaattcggcttactcactatagggctcgagcggccgcccgggcaggtcgccagtatgttggagttggtggtggcttaagttttgaagggaggtagcatccgttggatatccacaccatccttctcgctgcaggctttcttggactccgtactgttggtgtaaccaaggcctggaggtctgggtggctcaggtttcctgcagccatgtttctgtacaacttaaccttgcagagagccactggcatcagctttgccattcatggaaacttttctggaaccaaacaacaagaaattgttgtttcccgtgggaagatcttggagctgcttcgcccagaccccaacactggcaaagtacataccctactcactgtggaagtattcggtgttatccggtcactcatggcctttaggctgacaggtggcaccaaagactacattgtagttggcagtgactctggtcgaattgttattttggaataccagccatctaagaatatgtttgagaagattcaccaagaaacctttggcaagagtggatgccgtcgcatcgttcctggccagttcttagctgtggatcccaaagggcgagccgttatgattagtgccattgagaaacagaaattggtgtatattttgaacagagatgctgcagcccgacttaccatttcatctcccctggaagcccacaaagcaaacactttagtgtatcatgtagttggagtagatgtcggatttgaaaatccaatgtttgcttgtctggaaatggattatgaggaagcaggcaatgatccaacaggggaagcagcagctaatacccagcagacacttactttctatgagctagaccttggtttaaatcatgtggtccgaaaatacagtgaacctttggaggaacacggcaacttccttattacagttccaggagggtcagatggtccaagtggagtactgatctgctctgaaaactatattacttacaagaactttggtgaccagccagatatccgctgtccaattcccaggaggcggaatgacctggatgaccctgaaagaggaatgatttttgtctgctctgcaacccataaaaccaaatcgatgttcttcttttgggctcaaactgagcagggagatatctttaagatcactttggagacagatgaagatatggttactgagatccggctcaaatattttgatactgtacccgttgctgctgccatgtgtgtgcttaaaacagggttcctttttgtagcatcagaatttggaaaccattacttatatcaaattgcacatcttggagatgatgatgaagaacctgagttttcatcagccatgcctctggaagaaggagacacattcttttttcagccaagaccacttaaaaaccttgtgctggttgatgagttggacagcctctctcccattctgttttgccagatagctgatctggccaatgaagatactccacagttgtatgtggcctgtggtaggggaccccgatcatctctgagagtcctaagacatggacttgaggtgtcagaaatggctgtttctgagctacctggtaaccccaacgctgtctggacagtgcgtcgacacattgaagatgagtttgatgcctacatcattgtgtctttcgtgaatgccaccctagtgttgtccattggagaaactgtagaagaagtgactgactctgggttcctggggaccaccccgaccttgtcctgctccttattaggagatgatgccttggtgcaggtctatccagatggcattcggcacatacgagcagacaagagagtcaatgagtggaagacccctggaaagaaaacaattgtgaagtgtgcagtgaaccagcgacaagtggtgattgccctgacaggaggagagctggtctatttcgagatggatccttcaggacagctgaatgagtacacagaacggaaggagatgtcagcagatgtggtgtgcatgagtctggccaatgtaccccctggagagcagcggtctcgcttcctggctgtggggcttgtggacaacactgtcagaatcatctccctggatccctcagactgtttgcaacctctaagcatgcaggctctcccagcccagcctgagtccttgtgtatcgtggaaatgggtgggactgagaagcaggatgagctgggtgagaggggctcgattggcttcctatacctgaatattgggctacagaacggtgtgctgctgaggactgtcttggaccctgtcactggggatttgtctgatactcgcactcggtacctggggtcccgtcctgtgaagctcttccgagtccgaatgcaaggccaggaggcagtattggccatgtcaagccgctcatggttgagctattcttaccaatctcgcttccatctcaccccactgtcttacgagacactggaatttgcatcgggttttgcctcggaacagtgtcccgagggcattgtggccatctccaccaacaccctacggattttggcattagagaagctcggtgctgtcttcaatcaagtagccttcccactgcagtacacacccaggaaatttgtcatccaccctgagagtaacaaccttattatcattgaaacggaccacaatgcctacactgaggccacgaaagctcagagaaagcagcagatggcagaggaaatggtggaagcagcaggggaggatgagcgggagctggccgcagagatggcagcagcattcctcaatgaaaacctccctgaatccatctttggagctcccaaggctggcaatgggcagtgggcctctgtgatccgagtgatgaatcccattcaagggaacacactggaccttgtccagctggaacagaatgaggcagcttttagtgtggctgtgtgcaggttttccaacactggtgaagactggtatgtgctggtgggtgtggccaaggacctgatactaaacccccgatctgtggcagggggcttcgtctatacttacaagcttgtgaacaatggggaaaaactggagtttttgcacaagactcctgtggaagaggtccctgctgctattgccccattccaggggagggtgttgattggtgtggggaagctgttgcgtgtctatgacctgggaaagaagaagttactccgaaaatgtgagaataagcatattgccaattatatctctgggatccagactattggacatagggtaattgtatctgatgtccaagaaagtttcatctgggttcgctacaagcgtaatgaaaaccagcttatcatctttgctgatgatacctacccccgatgggtcactacagccagcctcctggactatgacactgtggctggggcagacaagtttggcaacatatgtgtggtgaggctcccacctaacaccaatgatgaagtagatgaggatcctacaggaaacaaagccctgtgggaccgtggcttgctcaatggggcctcccagaaggcagaggtgatcatgaactaccatgtcggggagacggtgctgtccttgcagaagaccacgctgatccctggaggctcagaatcacttgtctataccaccttgtctggaggaattggcatccttgtgccattcacgtcccatgaggaccatgacttcttccagcatgtggaaatgcacctgcggtctgaacatccccctctctgtgggcgggaccacctcagctttcgctcctactacttccctgtgaagaatgtgattgatggagacctctgtgagcagttcaattccatggaacccaacaaacaaaagaacgtctctgaagaactggaccgaaccccacccgaagtgtccaagaaactcgaggatatccggacccgctacgccttctgagccctcctttcccggtggggcttgccagagactgtgtgttttgtttcccccaccaccatcactgccacctggcttctgccatgtggcaggagggtgactggataattaagactgcattatgaaagtcaacagctctttcccctcagctcttctcctggaatgactggcttcccctcaaattggcactgagatttgctacacttctccccacctggtacatgatacatgaccccaggttccagtgtagaacctgagtcccccattccccaaagccatccctgcattgatatgtcttgactctcctgtctacttttgcacacacccttaatttttaattggttttcttgtaaatacagttttgtacaatgttatctctgtgggaggaaggaggcaggctgtggtgggactgggtagggtatagtatcactcctgagttccactgctctagaatctaaccagaaatagaaacctagtttttaaggtgactggcatccatgtgtcttgttctggagatgaggatgtaggtgggaggtttgaacccaagttagagcaggaagaactgagtagactccttccttccagataccgacttggacttgcggcactctgtggctccccacccccaggtctgtggtggtttctttgttttttcctggttctttttgctgtgctgatgaaacatgacctcaataaccatgtgtata

SEQ ID NO: 121 - DNA, 2505 bp, Homo sapiens
cgtttgcacctcgctgctccagcctctggggcgcattccaaccttccagcctgcgacctgcggagaaaaaaaaattacttattttcttgccccatacataccttgaggcgagcaaaaaattaaattttaaccatgagggaaatcgtgcacatccaggctggtcagtgtggcaaccagatcggtgccaagttctgggaggtgatcagtgatgaacatggcatcgaccccaccggcacctaccacggggacagcgacctgcagctggaccgcatctctgtgtactacaatgaagccacaggtggcaaatatgttcctcgtgccatcctggtggatctagaacctgggaccatggactctgttcgctcaggtccttttggccagatctttagaccagacaactttgtatttggtcagtctggggcaggtaacaactgggccaaaggccactacacagagggcgccgagctggttgattctgtcctggatgtggtacggaaggaggcagagagctgtgactgcctgcagggcttccagctgacccactcactgggcgggggcacaggctctggaatgggcactctccttatcagcaagatccgagaagaataccctgatcgcatcatgaataccttcagtgtggtgccttcacccaaagtgtctgacaccgtggtcgagccctacaatgccaccctctccgtccatcagttggtagagaatactgatgagacctattgcattgacaacgaggccctctatgatatctgcttccgcactctgaagctgaccacaccaacctacggggatctgaaccaccttgtctcagccaccatgagtggtgtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggaactcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctggggggggggtctagaacagtgcctggcacatagtaggcgctcaataaatacttgtttgttgaatgtctcctctctctttccactctgggaaacctaggtttctgccattctgggtgaccctgtatttctttctggtgcccattccatttgtccagttaatacttcctcttaaaaatctccaagaagctgggtctccagatcccatttagaaccaaccaggtgctgaaaacacatgtagataatggccatcatcctaagcccaaagtagaaaatggtagaaggtagtgggtagaagtcactatataaggaaggggatgggattttccattctaaaagttttggagagggaaatccaggctattaaagtcactaaatttctaagtatgtccatttcccatctcagcttcaagggaggtgtcagcagtattatctccactttcaatctccctccaagctctactctggaggagtctgtcccactctgtcaagtggaatccttccctttccaactctacctccctcactcagctcctttcccctgatcagagaaagggatcaagggggttgggaggggggaaagagaccagccttggtccctaagcctccagaaacgtcttcttaatccccaccttttcttactcccaaaaaagaatgaacacccctgactctggagtggtgtatactgccacatcagtgtttgagtcagtccccagaggagaggggaaccctcctccatcttttttgcaacatctcatttcttccttttgctgttgcttcccccctcacacacttggttttgttctatcctacatttgagatttctattttatgttgaacttgctgctttttttcatattgaaaagatgacatcgccccaagagccaaaaataaatgggaattgaaaaaaaaaaaaaaaaa

SEQ ID NO: 122 - DNA, 3600 bp, Homo sapiens
cgggcagctggtaccgaaggcggaggtggagcccgagagcagcggaagatgtcggacagcgaggacagcaacttttccgaggaggaggacagcgagcgcagcagtgacggcgaggaggccgaggtagacgaagagcggcggagtgcagcgggcagtgagaaagaagaagagcctgaggacgaagaggaggaggaagaggaggaggaatatgatgaggaagaggaggaagaagatgatgaccgaccccccaagaaaccccgccatggaggcttcattctggacgaggctgatgttgacgatgagtatgaggacgaggaccagtgggaggatggagcagaggacattctagagaaagaagagattgaagcctccaatatcgataatgttgtcctggatgaagatcgttctggggctcgccgcctgcaaaacctctggagggaccagcgagaagaagaactgggcgagtattacatgaagaaatacgccaagtcatctgtgggagagacggtgtatggaggatctgatgagctctcagacgacatcacccagcagcagctgctcccaggagtcaaggatcccaatctgtggattgtcaaatgtaagattggggaggaacgggccacggccatttccttgatgcgcaagttcattgcctaccagttcacagacacgcccctgcagatcaagtcagtagtggcaccagagcatgtgaagggctacatctacgtggaggcctacaagcagacccacgtgaagcaggccattgagggggtgggcaacctgcggcttggctactggaaccagcagatggtgcccatcaaggagatgacagacgtgctcaaagtggtgaaggaggtggccaacctgaaaccaaagtcctgggtccgcctcaagcggggcatctacaaggatgacattgctcaggtggactacgtggagcccagccagaacaccatctccctgaagatgatcccacgcatcgactacgatcgcatcaaggcccgcatgagcttgaaagactggtttgccaaaaggaagaagtttaagcggcctccacagaggctgtttgatgctgagaagatcaggtccctggggggtgatgttgcctctgatggtgacttcctcatctttgaggggaaccgttacagccggaagggctttctgttcaagagcttcgccatgtctgctgtgatcacggagggtgtgaagccaacactctctgagctggaaaagtttgaggaccagccagagggcattgacctggaggtggtgactgagagcacagggaaggagcgggagcacaacttccaacctggggacaacgtggaggtctgtgagggtgagctcatcaacctgcagggcaagatcctcagcgtggatggcaacaagatcaccatcatgcccaagcatgaggacctcaaggacatgttggagttcccagcccaggaacttagaaaatacttcaagatgggggaccacgtgaaggtgattgctgcccgattcgagggcgacacaggcctcattgtgcgggtggaggagaatttcgttatcctgttctctgacctcaccatgcatgagctgaaggtgctcccccgggacctgcagctctgctcagagacagcatcaggtgtggatgttgggggccagcatgaatggggcgagctggtgcagctggatccccagactgtgggtgtcatcgtgcgactagaacgggagaccttccaggtgctgaacatgtacgggaaggtggtgactgtcagacatcaggctgtgacccggaagaaggacaaccgctttgctgtggccttggactcagagcagaacaacatccatgtgaaagacatcgttaaggtcattgatggcccccactcaggccgagaaggggagattcgccatctcttccgaagcttcgccttcctacattgcaagaaactggtggagaacgggggcatgtttgtctgcaagacccgccacctggtgctggctgggggctcaaagccccgtgatgtgaccaacttcaccgtgggtggctttgcgcctatgagtccccggatcagcagccccatgcaccccagtgctggaggtcagcgtggcggctttggtagcccaggtggcggcagtggtggcatgagcaggggccggggccggagggacaacgaactcatcggccagaccgtgcgcatctcccaggggccctacaaaggctacatcggtgtggtgaaagatgccacagagtccacggcccgtgtggagctgcactccacctgccagaccatctctgtggaccgtcagcggctcaccacggtgggctcacggcgcccgggcggcatgacctcgacctatgggaggacgcccatgtatggctcccagacgcccatgtatggctctggctcccgaacacccatgtacggctcacagacacccctccaggatggtagccgcaccccacactacggatcacagacgcccctgcatgatggcagccgcactcctgcccagagtgggggctgggaccccaacaaccccaacacgccgtcacgggctgaggaagaatatgagtatgctttcgatgatgagcccaccccgtccccgcaggcctatgggggaacccccaatccccaaacacctggctacccagacccctcgtccccacaggtcaacccacaatacaacccgcagacgccagggacgccggccatgtacaacacagaccagttctctccctatgctgccccctccccacaaggttcctaccagcccagccccagcccccagagctaccaccaggtggcgccaagcccagcaggctaccagaatacccactccccagccagctaccaccctacaccgtcgcccatggcctatcaggctagccccagcccgagccccgttggctacagtcctatgacacctggagctccctcccctggtggctacaacccacacacgccaggctcaggcatcgagcagaactccagcgactgggtaaccactgacattcaggtgaaggtgcgggacacctacctggatacacaggtggtgggacagacaggtgtcatccgcagtgtcacggggggcatgtgctctgtgtacctgaaggacagtgagaaggttgtcagcatttccagtgagcacctggagcctatcacccccaccaagaacaacaaggtgaaagtgatcctgggcgaggatcgggaagccacgggcgtcctactgagtattgatggtgaggatggcattgtccgtatggaccttgatgagcagctcaagatcctcaacctccgcttcctggggaagctcctggaagcctgaagcaggcagggccggtggacttcgtcggatgaagagtgatcctccttccttccctggcccttggctgtgacacaagatcctcctgcagggctaggcggattgttctggatttccttttgtttttccttttagttttccatcttttccctccctggtgctcattggaatctgagtagagtctgggggagggtccccaccttcctgtacctcctccccacagcttgcttttgttgtaccgtctttcaataaaaagaagctgtttggtctaaaaaaaaaaaaaaaaaaaaa

SEQ ID NO: 123 - DNA, 9081 bp, Homo sapiens
gagcccagtcgagccgcgctcacctcgggctcccgctccgtctccacctccgcctttgccctggcggcgcgaccccgtcccgggcgcggccccagcagtcgcgcgccgttagcctcgcgcccgccgcgcagctccgggcccggcgcgatgggggccgccgccggccggagcccccacctggggcccgcgcccgcccgccgcccgcagcgctctctgctcctgctgcagctgctgctgctcgtcgctgccccggggtccacgcaggcccaggccgccccgttccccgagctgtgcagttatacatgggaagctgttgataccaaaaataatgtactttataaaatcaacatctgtggaagtgtggatattgtccagtgcgggccatcaagtgctgtttgtatgcacgacttgaagacacgcacttatcattcagtgggtgactctgttttgagaagtgcaaccagatctctcctggaattcaacacaacagtgagctgtgaccagcaaggcacaaatcacagagtccagagcagcattgccttcctgtgtgggaaaaccctgggaactcctgaatttgtaactgcaacagaatgtgtgcactactttgagtggaggaccactgcagcctgcaagaaagacatatttaaagcaaataaggaggtgccatgctatgtgtttgatgaagagttgaggaagcatgatctcaatcctctgatcaagcttagtggtgcctacttggtggatgactccgatccggacacttctctattcatcaatgtttgtagagacatagacacactacgagacccaggttcacagctgcgggcctgtccccccggcactgccgcctgcctggtaagaggacaccaggcgtttgatgttggccagccccgggacggactgaaggtggtgcgcaaggacaggcttgtcctgagttacgtgagggaagaggcaggaaagctagacttttgtgatggtcacagccctgcggtgactattacatttgtttgcccgtcggagcggagagagggcaccattcccaaactcacagctaaatccaactgccgctatgaaattgagtggattactgagtatgcctgccacagagattacctggaaagtaaaacttgttctctgagcggcgagcagcaggatgtctccatagacctcacaccacttgcccagagcggaggttcatcctatatttcagatggaaaagaatatttgttttatttgaatgtctgtggagaaactgaaatacagttctgtaataaaaaacaagctgcagtttgccaagtgaaaaagagcgatacctctcaagtcaaagcagcaggaagataccacaatcagaccctccgatattcggatggagacctcaccttgatatattttggaggtgatgaatgcagctcagggtttcagcggatgagcgtcataaactttgagtgcaataaaaccgcaggtaacgatgggaaaggaactcctgtattcacaggggaggttgactgcacctacttcttcacatgggacacggaatacgcctgtgttaaggagaaggaagacctcctctgcggtgccaccgacgggaagaagcgctatgacctgtccgcgctggtccgccatgcagaaccagagcagaattgggaagctgtggatggcagtcagacggaaacaaagaagaagcattttttcattaatatttgtcacagagtgctgcaggaaggcaaggcacgagggtgtcccgaggacgcggcagtgtgtgcagtggataaaaatggaagtaaaaatctgggaaaatttatttcctctcccatgaaagagaaaggaaacattcaactctcttattcagatggtgatgattgtggtcatggcaagaaaattaaaactaatatcacacttgtatgcaagccaggtgatctggaaagtgcaccagtgttgagaacttctggggaaggcggttgcttttatgagtttgagtggcacacagctgcggcctgtgtgctgtctaagacagaaggggagaactgcacggtctttgactcccaggcagggttttcttttgacttatcacctctcacaaagaaaaatggtgcctataaagttgagacaaagaagtatgacttttatataaatgtgtgtggcccggtgtctgtgagcccctgtcagccagactcaggagcctgccaggtggcaaaaagtgatgagaagacttggaacttgggtctgagtaatgcgaagctttcatattatgatgggatgatccaactgaactacagaggcggcacgccctataacaatgaaagacacacaccgagagctacgctcatcacctttctctgtgatcgagacgcgggagtgggcttccctgaatatcaggaagaggataactccacctacaacttccggtggtacaccagctatgcctgcccggaggagcccctggaatgcgtagtgaccgacccctccacgctggagcagtacgacctctccagtctggcaaaatctgaaggtggccttggaggaaactggtatgccatggacaactcaggggaacatgtcacgtggaggaaatactacattaacgtgtgtcggcctctgaatccagtgccgggctgcaaccgatatgcatcggcttgccagatgaagtatgaaaaagatcagggctccttcactgaagtggcttccatcagtaacttgggaatggcaaagaccggcccggtggttgaggacagcggcagcctccttctggaatacgtgaatgggtcggcctgcaccaccagcgatggcagacagaccacatataccacgaggatccatctcgtctgctccaggggcaggctgaacagccaccccatcttttctctcaactgggagtgtgtggtcagtttcctgtggaacacagaggctgcctgtcccattcagacaacgacggatacagaccaggcttgctctataagggatcccaacagtggatttgtgtttaatcttaatccgctaaacagttcgcaaggatataacgtctctggcattggtaagatttttatgtttaatgtctgcggcacaatgcctgtctgtgggaccatcctgggaaaacctgcttctggctgtgaggcagaaacccaaactgaagagctcaagaattggaagccagcaaggccagtcggaattgagaaaagcctccagctgtccacagagggcttcatcactctgacctacaaagggcctctctctgccaaaggtaccgctgatgcttttatcgtccgctttgtttgcaatgatgatgtttactcagggcccctcaaattcctgcatcaagatatcgactctgggcaagggatccgaaacacttactttgagtttgaaaccgcgttggcctgtgttccttctccagtggactgccaagtcaccgacctggctggaaatgagtacgacctgactggcctaagcacagtcaggaaaccttggacggctgttgacacctctgtcgatgggagaaagaggactttctatttgagcgtttgcaatcctctcccttacattcctggatgccagggcagcgcagtggggtcttgcttagtgtcagaaggcaatagctggaatctgggtgtggtgcagatgagtccccaagccgcggcgaatggatctttgagcatcatgtatgtcaacggtgacaagtgtgggaaccagcgcttctccaccaggatcacgtttgagtgtgctcagatatcgggctcaccagcatttcagcttcaggatggttgtgagtacgtgtttatctggagaactgtggaagcctgtcccgttgtcagagtggaaggggacaactgtgaggtgaaagacccaaggcatggcaacttgtatgacctgaagcccctgggcctcaacgacaccatcgtgagcgctggcgaatacacttattacttccgggtctgtgggaagctttcctcagacgtctgccccacaagtgacaagtccaaggtggtctcctcatgtcaggaaaagcgggaaccgcagggatttcacaaagtggcaggtctcctgactcagaagctaacttatgaaaatggcttgttaaaaatgaacttcacggggggggacacttgccataaggtttatcagcgctccacagccatcttcttctactgtgaccgcggcacccagcggccagtatttctaaaggagacttcagattgttcctacttgtttgagtggcgaacgcagtatgcctgcccacctttcgatctgactgaatgttcattcaaagatggggctggcaactccttcgacctctcgtccctgtcaaggtacagtgacaactgggaagccatcactgggacgggggacccggagcactacctcatcaatgtctgcaagtctctggccccgcaggctggcactgagccgtgccctccagaagcagccgcgtgtctgctgggtggctccaagcccgtgaacctcggcagggtaagggacggacctcagtggagagatggcataattgtcctgaaatacgttgatggcgacttatgtccagatgggattcggaaaaagtcaaccaccatccgattcacctgcagcgagagccaagtgaactccaggcccatgttcatcaacgccgtggaggactgtgagtacacctttgcctggcccacagccacagcctgtcccatgaagagcaacgagcatgatgactgccaggtcaccaacccaagcacaggacacctgtttgatctgagctccttaagtggcagggcgggattcacagctgcttacagcgagaaggggttggtttacatgagcatctgtggggagaatgaaaactgccctcctggcgtgggggcctgctttggacagaccaggattagcgtgggcaaggccaacaagaggctgagatacgtggaccaggtcctgcagctggtgtacaaggatgggtccccttgtccctccaaatccggcctgagctataagagtgtgatcagtttcgtgtgcaggcctgaggccgggccaaccaataggcccatgctcatctccctggacaagcagacatgcactctcttcttctcctggcacacgccgctggcctgcgagcaagcgaccgaatgttccgtgaggaatggaagctctattgttgacttgtctccccttattcatcgcactggtggttatgaggcttatgatgagagtgaggatgatgcctccgataccaaccctgatttctacatcaatatttgtcagccactaaatcccatgcacggagtgccctgtcctgccggagccgctgtgtgcaaagttcctattgatggtccccccatagatatcggccgggtagcaggaccaccaatactcaatccaatagcaaatgagatttacttgaattttgaaagcagtactccttgcttagcggacaagcatttcaactacacctcgctcatcgcgtttcactgtaagagaggtgtgagcatgggaacgcctaagctgttaaggaccagcgagtgcgactttgtgttcgaatgggagactcctgtcgtctgtcctgatgaagtgaggatggatggctgtaccctgacagatgagcagctcctctacagcttcaacttgtccagcctttccacgagcacctttaaggtgactcgcgactcgcgcacctacagcgttggggtgtgcacctttgcagtcgggccagaacaaggaggctgtaaggacggaggagtctgtctgctctcaggcaccaagggggcatcctttggacggctgcaatcaatgaaactggattacaggcaccaggatgaagcggtcgttttaagttacgtgaatggtgatcgttgccctccagaaaccgatgacggcgtcccctgtgtcttccccttcatattcaatgggaagagctacgaggagtgcatcatagagagcagggcgaagctgtggtgtagcacaactgcggactacgacagagaccacgagtggggcttctgcagacactcaaacagctaccggacatccagcatcatatttaagtgtgatgaagatgaggacattgggaggccacaagtcttcagtgaagtgcgtgggtgtgatgtgacatttgagtggaaaacaaaagttgtctgccctccaaagaagttggagtgcaaattcgtccagaaacacaaaacctacgacctgcggctgctctcctctctcaccgggtcctggtccctggtccacaacggagtctcgtactatatgaatctgtgccagaaaatatataaagggcccctgggctgctctgaaagggccagcatttgcagaaggaccacaactggagacgtccaggtcctgggactcgttcacacgcagaagctgggtgtcataggtgacaaagttgttgtcatgtactccaaaggttatccgtgtggtggaaataagaccgcatcctccgtgatagaattgacctgtacaaagacggtgggcagacctgcattcaagaggtttgatatcgacagctgcacttactacttcagctgggactcccgggctgcctgcgccgtgaagcctcaggaggtgcagatggtgaatgggaccatcaccaaccctataaatggcaagagcttcagcctcggagatatttattttaacctgttcagagaatctggggacatgaggaccaatggggacaactacctgtatgagatcctactttcctccatcacaagctccagaaacccggcgtgctctggagccaacatatgccaggtgaagcccaacgatcagcacttcagtcggaaagttggaacctctgacaagaccaagtactaccttcaagacggcgatctcgatgtcgtgtttgcctcttcctctaagtgcggaaaggataagaccaagtctgtttcttccaccatcttcttccactgtgaccctctggtggaggacgggatccccgagttcagtcacgagactgccgactgccagtacctcttctcttggtacacctcagccgtgtgtcctctgggggtgggctttgacagcgagaatcccggggacgacgggcagatgcacaaggggctgtcagaacggagccaggcagtcggcgcggtgctcagcctgctgctggtggcgctcacctgctgcctgctggccctgttgctctacaagaagaagaggagggaaacaatgataagtaagctgacctcttgctgtaggagaagttccaacgtgtcctacaaatactcaaaggtgaataaggaagaagagacagatgagaatgaaacagagtggctgatggaagagatccagctgcctcctccacggcagggaaaggaagggcaggagaacggccatattaccaccaagtcagtgaaagccctcagctccctgcatggggatgaccaggacagtacggatgaggttctgaccatcccagaggtgaaagttcactcgggcaggggagctggggcagagagctcccacccagtgagaaacgcacagagcaatgcccttcaggagcgtgaggacgatagggtggggctggtcaggggtgagaaggcgaggaaagggaagtccagctctgcacagcagaagacagtgagctccaccaagctggtgtccttccatgacgacagcgacgaggacctcttacacatctgactccgcagtgcctgcaggggagcacggagccgcgggacagccaagcacctccaaccaaataagacttccactcgatgatgcttctataattttgcctttaacagaaactttcaaaagggaagagtttttgtgatgggggagagggtgaaggaggtcaggccccactccttcctgattgtttacagtcattggaataaggcatggctcagatcggccacagggcggtaccttgtgcccagggttttgccccaagtcctcatttaaaagcataaggccggacgcatctcaaaacagagggctgcattcgaagaaacccttgctgctttagtcccgataggtatttgaccccgatatattttagcattttaattctctccccctatttattgactttgacaattactcaggtttgagaaaaaggaaaaaaaaacagccaccgtttcttcctgccagcaggggtgtgatgtaccagtttgtccatcttgagatggtgaggctgtcagtgtatggggcagcttccggcgggatgttgaactggtcattaatgtgtcccctgagttggagctcattctgtctcttttctcttttgctttctgtttcttaagggcacacacacgtgcgtgcgagcacacacacatacgtgcacagggtccccgagtgcctaggttttggagagtttgcctgttctatgcctttagtcaggaatggctgcacctttttgcatgatatcttcaagcctgggcgtacagagcacatttgtcagtatttttgccggctggtgaattcaacctgcccaaagattgatttgtgtgtttgtgtgtgtgtgtgtgtgtgtgtgtgtgtgtgtgagtggagttgaggtgtcagagaaaatgaattttttccagatttggggtataggtctcatctcttcaggttctcatgataccacctttactgtgcttatttttttaagaaaaaagtgttgatcaaccattcgacctataagaagccttaatttgcacagtgtgtgacttacagaaactgcatgaaaaatcatgggccagagcctcggccctagcattgcacttggcctcatgctggagggaggctgggcgggtacagcgcggaggaggagggaggccaggcgggcatggcgtggaggaggtgggaggccgggcggtcacagcatggaggaggagggaggcgctgctggtgttcttattctggcggcagcgcctttcctgccatgtttagtgaatgacttttctcgcattgtagaattgtatatagactctggtgttctattgctgagaagcaaaccgccctgcagcatccctcagcctgtaccggtttggctggcttgtttgatttcaacatgagtgtattttttaaaattgatttttctcttcatttttttttcaatcaactttactgtaatataaagtattcaacaatttcaataaaagataaattatt

SEQ ID NO: 124 - DNA, 1371 bp, Homo sapiens
ctgcaggggggggggggggctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctgg

SEQ ID NO: 125 - DNA, 2748 bp, Homo sapiens
gtcagacggcgctatgtacgccctcttcctcctggccagcctcctgggcgcggctctagccggcccggtccttggactgaaagaatgcaccaggggctcggcagtgtggtgccagaatgtgaagacggcgtccgactgcggggcagtgaagcactgcctgcagaccgtttggaacaagccaacagtgaaatcccttccctgcgacatatgcaaagacgttgtcaccgcagctggtgatatgctgaaggacaatgccactgaggaggagatccttgtttacttggagaagacctgtgactggcttccgaaaccgaacatgtctgcttcatgcaaggagatagtggactcctacctccctgtcatcctggacatcattaaaggagaaatgagccgtcctggggaggtgtgctctgctctcaacctctgcgagtctctccagaagcacctagcagagctgaatcaccagaagcagctggagtccaataagatcccagagctggacatgactgaggtggtggcccccttcatggccaacatccctctcctcctctaccctcaggacggcccccgcagcaagccccagccaaaggataatggggacgtttgccaggactgcattcagatggtgactgacatccagactgctgtacggaccaactccacctttgtccaggccttggtggaacatgtcaaggaggagtgtgaccgcctgggccctggcatggccgacatatgcaagaactatatcagccagtattctgaaattgctatccagatgatgatgcacatgcaacccaaggagatctgtgcgctggttgggttctgtgatgaggtgaaagagatgcccatgcagactctggtccccgccaaagtggcctccaagaatgtcatccctgccctggaactggtggagcccattaagaagcacgaggtcccagcaaagtctgatgtttactgtgaggtgtgtgaattcctggtgaaggaggtgaccaagctgattgacaacaacaagactgagaaagaaatactcgacgcttttgacaaaatgtgctcgaagctgccgaagtccctgtcggaagagtgccaggaggtggtggacacgtacggcagctccatcctgtccatcctgctggaggaggtcagccctgagctggtgtgcagcatgctgcacctctgctctggcacgcggctgcctgcactgaccgttcacgtgactcagccaaaggacggtggcttctgcgaagtgtgcaagaagctggtgggttatttggatcgcaacctggagaaaaacagcaccaagcaggagatcctggctgctcttgagaaaggctgcagcttcctgccagacccttaccagaagcagtgtgatcagtttgtggcagagtacgagcccgtgctgatcgagatcctggtggaggtgatggatccttccttcgtgtgcttgaaaattggagcctgcccctcggcccataagcccttgttgggaactgagaagtgtatatggggcccaagctactggtgccagaacacagagacagcagcccagtgcaatgctgtcgagcattgcaaacgccatgtgtggaactaggaggaggaatattccatcttggcagaaaccacagcattggtttttttctacttgtgtgtctgggggaatgaacgcacagatctgtttgactttgttataaaaatagggctcccccacctcccccatttctgtgtcctttattgtagcattgctgtctgcaagggagcccctagcccctggcagacatagctgcttcagtgccccttttctctctgctagatggatgttgatgcactggaggtcttttagcctgcccttgcatggcgcctgctggaggaggagagagctctgctggcatgagccacagtttcttgactggaggccatcaaccctcttggttgaggccttgttctgagccctgacatgtgcttgggcactggtgggcctgggcttctgaggtggcctcctgccctgatcagggaccctccccgctttcctgggcctctcagttgaacaaagcagcaaaacaaaggcagttttatatgaaagattagaagcctggaataatcaggctttttaaatgatgtaattcccactgtaatagcatagggattttggaagcagctgctggtggcttgggacatcagtggggccaagggttctctgtccctggttcaactgtgatttggctttcccgtgtctttcctggtgatgccttgtttggggttctgtgggtttgggtgggaagagggccatctgcctgaatgtaacctgctagctctccgaaggccctgcgggcctggcttgtgtgagcgtgtggacagtggtggccgcgctgtgcctgctcgtgttgcctacatgtccctggctgttgaggcgctgcttcagcctgcacccctcccttgtctcatagatgctccttttgaccttttcaaataaatatggatggcgagctcctaggcctctggcttcctggtagagggcggcatgccgaagggtctgctgggtgtggattggatgctggggtgtgggggttggaagctgtctgtggcccacttgggcacccacgcttctgtccacttctggttgccaggagacagcaagcaaagccagcaggacatgaagttgctattaaatggacttcgtgatttttgttttgcactaaagtttctgtgatttaacaataaaattctgttagccagaaaaaaaaaaaaaaaaaa

SEQ ID NO: 126 - DNA, 1071 bp, Homo sapiens
gcagttctggtcctcctaggagcggccgcctgcgcggcgcggccccgtggtcggatgctgggcggcagagaggccgaggcgcacgcgcggccctacatggcgtcggtgcagctgaacggcgcgcacctgtgcgcaggcgtcctggtggcggagcggtgggtgctgagcgcggcgcactgcctggaggacgcggccgacgggaaggtgcaggttctcctgggcgcgcactccctgtcgcagccggagccctccaagcgcctgtacgacgtgctccgcgcagtgccccacccggacagccagcccgacaccatcgaccacgacctcctgctgctacagctgtcggagaaggccacactgggccctgctgtgcgccccctgccctggcagcgcgtggaccgcgacgtggcaccgggaactctctgcgacgtggccggctggggcatagtcaaccacgcgggccgccgcccggacagcctgcagcacgtgctcttgccagtgctggaccgcgccacctgcaaccggcgcacgcaccacgacggcgccatcaccgagcgcttgatgtgcgcggagagcaatcgccgggacagctgcaagggtgactccgggggcccgctggtgtgcgggggcgtgctcgagggcgtggtcacctcgggctcgcgcgtttgcggcaaccgcaagaagcccgggatctacacccgcgtggcgagctatgcggcctggatcgacagcgtcctggcctagggtgccggggcctgaaggtcagggtcacccaagcaacaaagtcccgagcaatgaagtcatccactcctgcatctggttggtctttattgagcacctactatatgcagaaggggaggccgaggtgggaggatcattggatctcaggagttggagatcagcatgggccacgtagcgcgactccatctctacaaataaataaaaattagctgggcaattggcgggcatggaggtgggtgcttgtagttccagctactcaggaggctgaggtgggaggatgacttgaacgcaggaggctgaggctgcagtgagttgtgattgcaccactgccct

SEQ ID NO: 127 - DNA, 8707 bp, Homo sapiens
gaggatcacgtggcgcggcgccgcggccgaagcagaagtagcgagcgccggcggcggagggcgtgagcggcgctgagtgacccgagtcgggacgcgggctgcgcgcgcgggaccccggagcccaaacccggggcaggcgggcagctgtgcccgggcggcacggccagcttcctgatttctcccgattccttccttctccctggagcggccgacaatgtccaccacggtcaatgtagattcccttgcggaatatgagaagagccagatcaagagagccctggagctggggacggtgatgactgtgttcagcttccgcaagtccacccccgagcggagaaccgtccaggtgatcatggagacgcggcaggtggcctggagcaagaccgctgacaagatcgagggcttcttggatatcatggaaataaaagaaatccgcccagggaagaactccaaagatttcgagcgagcaaaagcagttcgccagaaagaagactgctgcttcaccatcctatatggcactcagttcgtcctcagcacgctcagcttggcagctgactctaaagaggatgcagttaactggctctctggcttgaaaatcttacaccaggaagcgatgaatgcgtccacgcccaccattatcgagagttggctgagaaagcagatatattctgtggatcaaaccagaagaaacagcatcagtctccgagagttgaagaccatcttgcccctgatcaactttaaagtgagcagtgccaagttccttaaagataagtttgtggaaataggagcacacaaagatgagctcagctttgaacagttccatctcttctataaaaaacttatgtttgaacagcaaaaatcgattctcgatgaattcaaaaaggattcgtccgtgttcatcctggggaacactgacaggccggatgcctctgctgtttacctgcatgacttccagaggtttctcatacatgaacagcaggagcattgggctcaggatctgaacaaagtccgtgagcggatgacaaagttcattgatgacaccatgcgtgaaactgctgagcctttcttgtttgtggatgagttcctcacgtacctgttttcacgagaaaacagcatctgggatgagaagtatgacgcggtggacatgcaggacatgaacaaccccctgtctcattactggatctcctcgtcacataacacgtaccttacaggtgaccagctgcggagcgagtcgtccccagaagcttacatccgctgcctgcgcatgggctgtcgctgcattgaactggactgctgggacgggcccgatgggaagccggtcatctaccatggctggacgcggactaccaagatcaagtttgacgacgtcgtgcaggccatcaaagaccacgcctttgttacctcgagcttcccagtgatcctgtccatcgaggagcactgcagcgtggagcaacagcgtcacatggccaaggccttcaaggaagtatttggcgacctgctgttgacgaagcccacggaggccagtgctgaccagctgccctcgcccagccagctgcgggagaagatcatcatcaagcataagaagctgggcccccgaggcgatgtggatgtcaacatggaggacaagaaggacgaacacaagcaacagggggagctgtacatgtgggattccattgaccagaaatggactcggcactactgcgccattgccgatgccaagctgtccttcagtgatgacattgaacagactatggaggaggaagtgccccaggatataccccctacagaactacattttggggagaaatggttccacaagaaggtggagaagaggacgagtgccgagaagttgctgcaggaatactgcatggagacggggggcaaggatggcaccttcctggttcgggagagcgagaccttccccaatgactacaccctgtccttctggcggtcaggccgggtccagcactgccggatccgctccaccatggagggcgggaccctgaaatactacttgactgacaacctcaccttcagcagcatctatgccctcatccagcactaccgcgagacgcacctgcgctgcgccgagttcgagctgcggctcacggaccctgtgcccaaccccaacccccacgagtccaagccgtggtactatgacagcctgagccgcggagaggcagaggacatgctgatgaggattccccgggacggggccttcctgatccggaagcgagaggggagcgactcctatgccatcaccttcagggctaggggcaaggtaaagcattgtcgcatcaaccgggacggccggcactttgtgctggggacctccgcctattttgagagtctggtggagctcgtcagttactacgagaagcattcactctaccgaaagatgagactgcgctaccccgtgacccccgagctcctggagcgctacaatatggaaagagatataaactccctctacgacgtcagcagaatgtatgtggatcccagtgaaatcaatccgtccatgcctcagagaaccgtgaaagctctgtatgactacaaagccaagcgaagcgatgagctgagcttctgccgtggtgccctcatccacaatgtctccaaggagcccgggggctggtggaaaggagactatggaaccaggatccagcagtacttcccatccaactacgtcgaggacatctcaactgcagacttcgaggagctagaaaagcagattattgaagacaatcccttagggtctctttgcagaggaatattggacctcaatacctataacgtcgtgaaagcccctcagggaaaaaaccagaagtcctttgtcttcatcctggagcccaagcagcagggcgatcctccggtggagtttgccacagacagggtggaggagctctttgagtggtttcagagcatccgagagatcacctggaagattgacaccaaggagaacaacatgaagtactgggagaagaaccagtccatcgccatcgagctctctgacctggttgtctactgcaaaccaaccagcaaaaccaaggacaacttagaaaatcctgacttccgagaaatccgctcctttgtggagacgaaggctgacagcatcatcagacagaagcccgtcgacctcctgaagtacaatcaaaagggcctgacccgcgtctacccaaagggacaaagagttgactcttcaaactacgaccccttccgcctctggctgtgcggttctcagatggtggcactcaatttccagacggcagataagtacatgcagatgaatcacgcattgttttctctcaatgggcgcacgggctacgttctgcagcctgagagcatgaggacagagaaatatgacccgatgccacccgagtcccagaggaagatcctgatgacgctgacagtcaaggttctcggtgctcgccatctccccaaacttggacgaagtattgcctgtccctttgtagaagtggagatctgtggagccgagtatgacaacaacaagttcaagacgacggttgtgaatgataatggcctcagccctatctgggctccaacacaggagaaggtgacatttgaaatttatgacccaaacctggcatttctgcgctttgtggtttatgaagaagatatgttcagcgatcccaactttcttgctcatgccacttaccccattaaagcagtcaaatcaggattcaggtccgttcctctgaagaatgggtacagcgaggacatagagctggcttccctcctggttttctgtgagatgcggccagtcctggagagcgaagaggaactttactcctcctgtcgccagctgaggaggcggcaagaagaactgaacaaccagctctttctgtatgacacacaccagaacttgcgcaatgccaaccgggatgccctggttaaagagttcagtgttaatgagaaccagctccagctgtaccaggagaaatgcaacaagaggttaagagagaagagagtcagcaacagcaagttttactcatagaagctggggtatgtgtgtaagggtattgtgtgtgtgcgcatgtgtgtttgcatgtaggagaacgtgccctattcacactctgggaagacgctaatctgtgacatcttttcttcaagcctgccatcaaggacatttcttaagacccaactggcatgagttggggtaatttcctattattttcatcttggacaactttcttaacttatattctttatagaggattccccaaaatgtgctcctcatttttggcctctcatgttccaaacctcattgaataaaagcaatgaaaaccttgatcaattaagccttctgttgcacgacctgtgcagtgaacaggatttcttttctggccaagaagattctacctctaatgatccaggtaactgatgtccatggaggatgagctggaaatgtaagaaactattcatgagattctgaaaaggattttaactcaaaggcaaatgattccataagggcccaaagagaagccctacccacaggcagcctgctcagttcaatgtactttaactaccaccggctgcctgctgcagtccacaagaaaatggctgagtgatgggatctgttcattaagacaatttctaattaatggtgacagcttgttttgtgactagagttactgggatggagggtaggaatcttggggcctctttgttttaaaaagcccatcagagagaccagagccgtgctgcaggggcaggttctcacttgcccctggctctgccagctgctgggaggctctggccccactagtccctcatggccctactgaactggctgggaggctgctggaatggcccttggtccacagctctccacaggcaagaggtcaactgctgcttgaaagaggtagacaaaagttaggttgatggcgaaatgtctctgggttacccagtcttctggagcagcaagctgagctttaatgggctaagcattagggtgttacagaaaatttcaaatgcagccatctcccttggggcagatctacctagttcatgacagtatgtgcggctggccagggctttacacctctgcatcttaagttgttaatacataccaataatgtaatatggctttttaaaggagaggagagtgctgggttgggaagggaggtggttggtagagtcacaacttctcaatgagtgaatttacagctgatgggaaaaggagtgtaactgtgaaaaacgatggctgtggtggggaagaacaaaccagcagtaagcctgatgtttgatgtggatggaactggcccctagaaacccatctgaccctcctcttgttacccgaaatgctgggcttagtatgcatgtactgctgaaaagcagggcagaacaaatcaggctctgaccagaagatccttctggtcccttcactctacaaaaacttactgatcacctccacatgccaaatacagtgccaagatttgggggtgtggatgtttaaacaaaaagctgtgggtctcatcaatcatctccatccacaagctcctaaaagaaagccatttacctcgcttgaagccaggaacacagggaacagcagtctggccaaggaagggctgttatctggtgctatcactccagttactcctccaactgggagctgctattttatttggcagtcagcaactgaagaaagaacattcctcttagtggcagatgttcaaagcaactttcaagaaaggctaggtgagaaaggcactgggatgagtgctgcaggcactctgtagccagggccccattagcctttggccaggtagccaccagaacctatttattgcacctggcatctcccccaacccctctcagctctgttaggacttccacacagcagagctcaggtgttgctgtcattacctcctttcagctcctcacttcattctactttaaagccacagtgctaaggcctgcatcccctttctgcccaaatgggttttttgctaccatatcaaagaacctgacatatggcggcataggaagcagaagctaagcctctctccagctgctgctgtgtaaaatccatgcgtggccaaagagaagtcaggggattatgacataaatggtgctgggaagaaccctctgcctaaaactgtctccttctcctggtgctacaaccggaatccaccatgagagagtactttcttcggttctttcctcctgtccttgacagagtaacacgttaatctggttcttggtggtgttagggactgattctctcaggaaaggcacacatggtatgatggctcttcccagagtctatgtgatgctacataacttcagtatctagctgagacatgcttcctacatgactgttaaagcacagccaatccaggccaagaagactagtaacaggcacattctgaaagatggaagcagcactgatagatcaaaaccaccactgcatatgtattacactgtttttgttcaccattttcctaagtgtgttatttagaatattggttattacaaggaaaaataaagtggggaggctggttaggccttgtgagtttgggaaacttaggttataaaaactaaataaagtttttctactgtgagactagatgtgcaggagtgaaaggtgtagagggtcttgttttccaaattcgatctcagaatctttttgccagaagtgtctcatgggacttatctatagtggaacacatttgaagacctactgctctattaagaaggcagccggacaacatgttctaatacttcgtatgctttgtgacctagttaaaatctaaacttaagtcgccatggccagtggcctttagattaagctagccttacccctgggagtataccagagctttccaaggaatacacagactccagtactctcaggggagcagtgttcagagcctcatcttcctgttatattcttctctaagattcatctgcctgagaaaatgcccttttctcaccttacaaaagaaaatatggctgtctccacctctagtcttactgtagagcatgtcccaaggtgtaaaaattcaaaatgtggatatttggaaagtgaaagacttatcaacagggcacaaatctttttgcaaatggattttccaagtttttctggtggttccaaattttttgctttcaacaaagtgggaggaacagcctgtagatttctgagtctcttagcatgtaactacaaaggggttggaagaattcagtgattctgctatcataaagcttccgttcccattgatgtatctgtgtgaacaaggatcaacatctccataaatgaaattgaaaacggaaaatagaattgatgatgaactttggctcaatcttaagatgttatcaatctacatagatgaaataattgtggagaaaagccctctttatctcattaagtgatacatttccaaagaagttttactatgtttaataatttagtgaaatttgggctatgtgtttattgattcagctcaatccagaggaaaattttaaaggcttacagccttaggattataggatactatataatacttttggtacagagatagaattaaataacataaaaatcaaaaatttattaggctaaaattttgagggagaagtggtatgaaaatacaaattcaaggagtaaaaggaaaagtggggcattccttgctactaaaaattgccttgttccaggtaagactgatcataaaaaaatggccctgttcataaaatttttaaaaagatcatagtatctatcaaataacttatattaagaacctcctgggctaaatttaaaaagtaatacaacagttttatttaaacatgtagtgtctacggtatgccagcactttgcagctatttataatgagaaattttagatgtcaatatagcaatgtgcaagaagatagagattttcaaaattcacttaagagtatctgagcataaaatgttaagattgctgatcggatgtgagggcgatctggctgcgacatctgtcaccccattgatcgccagggttgattcggctgatctggctggctaggtgggtgtccccttcctacctcaccgctccatgtgcgtccctcccgaagctgcgcgctccgtcgaagaggacgaccaaccccgatagaggaggaccggtcttcggtcaagggtatacgagtagctgcgctcccctgctggaacctccaaacaagctctcaagattgctgatctagggccactaagtgatgaattgtatttggaagcaaaaaggatggctaaaaaggacctcaacccttttgactttaaaaggaaaatagcttaaccttcaacctgtgtgacatttaactttttgaacccaaccgtaaaagctatcttctaaccaacaaaaagttaataattagatttggaattatacagaattagaaaattggcatttaaaaatactcaataatttgtccctggtttttaattttcaaaatattttctttttgaagagccagattccagtgatcctgcctctcagaaatttccacatttcttatttttcattaggccttaagaagctgcatttgtaaacttgtgtttcattattaaagcttaatttattttttatataaatagtatgtgctttgtgtacatagagaattaagtgaatgagtcacacagatgttggctgttgttaatgtgaaaattaaacagctgtatcacattttgaaaaataaaagtttcatctgaatgaatatagcaa

SEQ ID NO: 128 - DNA, 1470 bp, Homo sapiens
  modified_base (1454)..(1454): a, c, t, g, unknown or other
gcggccgcgtggagaggaggtggcagcggcccgggaggccggagccaagccagcgacccaccatggagacccgctacaacctgaagagtccggctgttaaacgtttaatgaaagaagcggcagaattgaaagatccaacagatcattaccatgcgcagcctttagaggataacctttttgaatggcacttcacggttagagggcccccagactccgattttgatggaggagtttatcacgggcggatagtactgccaccagagtatcccatgaaaccaccaagcattattctcctaacggctaatggtcgatttgaagtgggcaagaaaatctgtttgagcatctcaggccatcatcctgaaacttggcagccttcgtggagtataaggacagcattattagccatcattgggtttatgccaacaaaaggagagggagccataggttctctagattacactcctgaggaaagaagagcacttgccaaaaaatcacaagatttctgttgtgaaggatgtggctctgccatgaaggatgtcctgttgcctttaaaatctggaagcgattcaagccaagctgaccaagaagccaaagaactggctaggcaaataagctttaaggcagaagtcaattcatctggaaagactatctctgagtcagacttaaaccactctttttcactaactgatttacaagatgatatacctacaacattccagggtgctacggccagtacatcgtacggactccagaattcctcagcagcatcctttcatcaacctacccaacctgtagctaagaatacctccatgagccctcgacagcgccgggcccagcagcagagtcagagaaggttgtctacttcaccagatgtaatccagggccaccagccaagagacaaccacactgatcatggtgggtcagctgtactgattgtcatcctgactttggcattggcagctcttatattccgacgaatatatctggcaaacgaatacatatttgactttgagttataatatggttttgtgacttatgagctgtgactcaactgcttcattaaacattctgcattgggtataatctaagaattgtttacaaaaagattattttgtatttacccttcattcctttttttgatccttgtaagtttagtataaatatatctagacattcagactgtgtctagcagttacgtcctgcttaaagggactagaagtcaaagttccttgtctcactatttgatctgctttgcagggaaataacttgttttttctcatgtttcatcttctttttatgtaaatttgtaatactttcctatattgccctttgaaatttttggataaaagatgatgttttaagttccaatggagwaattactagttactcaataccacttattgagtaacctgtttcctacggaatgtagaatgtataggggaagaagaggttgnaaaggggaagccaaac

SEQ ID NO: 129 - DNA, 4518 bp, Homo sapiens
agaggctagcacggttcctgacaagtctagagcagcactagcgcagagtgagactcatcagctggcaagcaggattctgtgtctctgggttcaggaatcctcaggcagcagaggggacttgctcctcagcccccatcactgcccccacccctacccgtgtaggccaccgagtgaggcccatggactggggagggctgtgcctgacgactcctgtgtccctctatgagggcccagcgccctgaccctcctgcctaggtttctaccttttctctctgtctttggccagctgggggagggggtagaggccgggtgagcaacatggcacagagcaagaggcacgtgtacagccggacgcccagcggcagcaggatgagtgcggaggcaagcgcccggcctctgcgggtgggctcccgtgtagaggtgattggaaaaggccaccgaggcactgtggcctatgttggagccacactgtttgccactggcaaatgggtaggcgtgattctggatgaagcaaagggcaaaaatgatggaactgttcaaggcaggaagtacttcacttgtgatgaagggcatggcatctttgtgcgccagtcccagatccaggtatttgaagatggagcagatactacttccccagagacacctgattcttctgcttcaaaagtcctcaaaagagagggaactgatacaactgcaaagactagcaaactgcggggactgaagcctaagaaggcaccgacagcccgaaagaccacaactcggcgacccaagcccacgcgcccagccagtactggggtggctggggccagtagctccctgggcccctctggctcagcgtcagcaggtgagctgagcagcagtgagcccagcaccccggctcagactccgctggcagcacccatcatccccacgccggtcctcacctctcctggagcagtccccccgcttccttccccatccaaggaggaggagggactaagggctcaggtgcgggacctggaggagaaactagagaccctgagactgaaacgggcagaagacaaagcaaagctaaaagagctggagaaacacaaaatccagctggagcaggtgcaggaatggaagagcaaaatgcaggagcagcaggccgacctgcagcggcgcctcaaggaggcgagaaaggaagccaaggaggcgctggaggcaaaggaacgctatatggaggagatggctgatactgctgatgccattgagatggccactttggacaaggagatggctgaagagcgggctgagtccctgcagcaggaggtggaggcactgaaggagcgggtggacgagctcactactgacttagagatcctcaaggctgagattgaagagaagggctcagatggcgctgcatccagttatcagctcaagcagcttgaggagcagaatgcccgcctgaaggatgccctggtgaggatgcgggatctttcttcctcagagaagcaggagcatgtgaagctccagaagctcatggaaaagaagaaccaagagctggaagttgtgaggcaacagcgggagcgtctgcaggaggagctaagccaggcagagagcaccattgatgagctcaaggagcaggtggatgctgctctgggtgctgaggagatggtggagatgctgacagatcggaacctgaatctggaagagaaagtgcgcgagttgagggagactgtgggagacttggaagcgatgaatgagatgaacgatgagctgcaggagaatgcacgtgagacagaactggagctgcgggagcagctggacatggcaggcgcgcgggttcgtgaggcccagaagcgtgtggaggcagcccaggagacggttgcagactaccagcagaccatcaagaagtaccgccagctgaccgcccatctacaggatgtgaatcgggaactgacaaaccagcaggaagcatctgtggagaggcaacagcagccacctccagagacctttgacttcaaaatcaagtttgctgagactaaggcccatgccaaggcaattgagatggaattgaggcagatggaggtggcccaggccaatcgacacatgtccctgctgacagccttcatgcctgacagcttccttcggccaggtggggaccatgactgcgttctggtgctgttgctcatgcctcgtctcatttgcaaggcagagctgatccggaagcaggcccaggagaagtttgaactaagtgagaactgttcagagcggcctgggctgcgaggagctgctggggagcaactcagctttgctgctggactggtgtactcgctgagcctgctgcaggccacgctacaccgctatgagcatgccctctctcagtgcagtgtggatgtgtataagaaagtgggcagcctgtaccctgagatgagtgcccatgagcgctccttggatttcctcattgaactgctgcacaaggatcagctggatgagactgtcaatgtggagcctctcaccaaggccatcaagtactatcagcatctgtacagcatccaccttgccgaacagcctgaggactgtactatgcagctggctgaccacattaagttcacgcagagtgctctggactgcatgagtgtggaggtaggacggctgcgtgccttcttgcagggtgggcaggaggctacagatattgccctcctgctccgggatctggaaacttcatgcagtgacatccgccagttctgcaagaagatccgaaggcgaatgccagggacagatgctcctgggatcccagctgcactggcctttggaccacaggtatctgacacgctcctagactgcaggaaacacttgacgtgggtcgtggctgtgctgcaggaggtggcagctgctgctgcccagctcattgccccactggcagagaatgaggggctacttgtggctgctctggaggaactggctttcaaagcaagcgagcagatctatgggaccccctccagcagcccctatgagtgtctgcgccagtcatgcaacatcctcatcagtaccatgaacaagctggccacagccatgcaggagggggagtatgatgcagagcggccccccagcaagcctccaccggttgaactgcgggctgctgcccttcgtgcagagatcacagatgctgaaggcctgggtttgaagctcgaagatcgagagacagttattaaggagttgaagaagtcactcaagattaagggagaggagctaagtgaggccaatgtgcggctgagcctcctggagaagaagttggacagtgctgccaaggatgcagatgagcgcatcgagaaagtccagactcggctggaggagacccaggcactgctgcgaaagaaggagaaagagtttgaggagacaatggatgcactccaggctgacatcgaccagctggaggcagagaaggcagaactaaagcagcgtctgaacagccagtccaaacgcacgattgagggactccggggccctcctccttcaggcattgctactctggtctctggcattgctggtgaagaacagcagcgaggagccatccctgggcaggctccagggtctgtgccaggcccagggctggtgaaggactcaccactgctgcttcagcagatctctgccatgaggctgcacatctcccagctccagcatgagaacagcatcctcaagggagcccagatgaaggcatccttggcatccctgccccctctgcatgttgcaaagctatcccatgagggccctggcagtgagttaccagctggagcgctgtatcgtaagaccagccagctgctggagacattgaatcaattgagcacacacacgcacgtagtagacatcactcgcaccagccctgctgccaagagcccgtcggcccaacttatggagcaagtggctcagcttaagtccctgagtgacaccgtcgagaagctcaaggatgaggtcctcaaggagacagtatctcagcgccctggagccacagtacccactgactttgccaccttcccttcatcagccttcctcagggccaaggaggagcagcaggatgacacagtctacatgggcaaagtgaccttctcatgtgcggctggttttggacagcgacaccggctggtgctgacccaggagcagctgcaccagcttcacagtcgcctcatctcctaagcactcctttcccctgctgtccccttcgaccctcagccctctggtgccgctctgcccgatgcacagccacctcagccagcccccaggtagaaacgtgggttaagctcttcctgccccgttcagcttcactcccaccctttcagcgtcctgccccttcaccttgacccgggttcccccactcccattccctggcctctgccataatttgttgttcaactgctccctccttcctgaggggcctcagggcttgtggggggtaggctgagaccccaccaccaaaggttaagtgaggtccccttgattgaggacttcaccccttgattaaagcaacttctgcttcagtgcaaaaaaaaaaaaaaaaaa

SEQ ID NO: 130 - DNA, 1800 bp, Homo sapiens
acgaaggccggagaatggatttatcaggagtgaaaaagaagagcttgctaggagtcaaagaaaataataaaaagtccagcactagggctccttcacctaccaaacgcaaagaccgctcagatgagaagtccaaggatcgctcaaaagataaaggggccaccaaggagtcgagtgagaaggatcgcggccgggacaaaacccgaaagagaggcagcgcttccagtggtagcagcagtaccaggtctcggtccagctcgacttccagctcaggctccagcaccagcactggctcaagcagtggctccagctcttcctcagcatccagccgctcaggaagctccagcacctcccgcagctccagctctagcagctcttctggctctccaagtccttctcggcgcagacacgacaacaggaggcgctcccgctccaaatccaaaccacctaaaagagatgaaaaggagaggaaaaggcggagcccatctcctaagcccaccaaagtgcacattgggagactcacccggaatgtgacaaaggatcacatcatggagatattttccacctatgggaaaattaaaatgattgacatgcccgtggaaaggatgcatccccatctgtccaaaggctatgcgtacgtagagtttgagaatccagatgaagccgagaaggcgctgaagcacatggatggaggacaaattgatggccaggagatcactgccaccgccgtgctggccccctggcctaggccaccccccaggagattcagccctcccaggagaatgttgccaccaccgcctatgtggcgcaggtctcccccacggatgaggagaaggtcccgctccccgaggcgcaggtcccccgtgcgccggagatcacggtccccgggccgccgccgccacaggagccgctccagctccaactcctcccgataaacaggccactgaagctctcgcccctgtaacttataccccacccagctcagttttgtcacttttctagccaaaggaagaccagtaggaaagcaaacccttgactctggcaggatttgcaggcagcaggcagcacccctctgacggaccccggctgcagaagtgctgttggtttggatgctgtgtgcctgtcaagattcctccggttttctggctagaaagctcatcgtttccggtttctaagagtcagttcatgcagagccaccagggaaaagtgaggctcttgggggtggtttgaccctgcttacctgggagcacacttttcccttccccgatgacctgggatggtggccaggccgtgcccttgctgttgctgggcagtgtccttttggaaagggagaaacccaggctttagtgtagctgccaaccctgttaggcctccgtctcgaggcctcttctgacctcaaggtcacaccccctcaaagatcctctcacccatggtagttgctgctcgtggttctgtctgtccgtgcaccgatgcacacaccgcaccccaccactgtactctgaaattggcgagtgactgtactctgaatggcgagtgagttgagagccagctctgcggggtcatcacgcagccatggttgtgcctgccgttcatggtggtctttcaggttatcttggcaacatgtacattgcttttattttttttcttttttgctttcattgtacagctagtactataaaatttctcttttgagttttatacctttgtagcattttagatgacattgtgtttgtactttgttgtgtagagtggaagaattgtgttgaataaacccaagatcggaatgcaaaaaaaaaaaaaaaa

SEQ ID NO: 131 - DNA, 899 bp, Homo sapiens
catctgagtaacatggcggcggcggcggtagccaggctgtggtggcgcgggatcttgggggcctcggcgctgaccagggggactgggcgaccctccgttctgttgctgccggtgaggcgggagagcgccggggccgacacgcgccccactgtcagaccacggaatgatgtggcccacaagcagctctcagcttttggagagtatgtggctgaaatcttgcccaagtatgtccaacaagttcaggtgtcctgcttcaatgagttagaggtctgtatccatcctgatggcgtcatcccagtgctgactttcctcagggatcacaccaatgcacagttcaaatctctggttgacttgacagcagtggacgtcccaactcggcaaaaccgttttgagattgtctacaacctgttgtctctgcgcttcaactcacggatccgtgtgaagacctacacagatgagctgacgcccattgagtctgctgtctctgtgttcaaggcagccaactggtatgaaagggagatctgggacatgtttggagtcttctttgctaaccaccctgatctaagaaggatcctgacagattatggcttcgagggacatcctttccggaaagactttcctctatctggctatgttgagttacgttatgatgatgaagtgaagcgggtggtggcagagccggtggagttggcccaagagttccgcaaatttgacctgaacagcccctgggaggctttcccagtctatcgccaacccccggagagtctcaagcttgaagccggagacaagaagcctgatgccaagtagctccagggaacgcatgtggatcctagacagcgccttatctatgattgagtgtccgtgtaaataaattcctacttagacttaaaaaaaaaaaa

SEQ ID NO: 132 - DNA, 1647 bp, Homo sapiens
gaattccggaaaacaggccgcgcgggcggcagaggagccgggcgccgcaatggacgtgcgggcgctgccgtggctgccgtggctgctgtggctgctgtgccggggcggcggcgatgcggactcccgcgcccccttcaccccgacctggccgcggagccgcgagcgtgaagccgccgccttccgggaaagtcttaatagacatcgatacttgaattctttatttcccagtgaaaactccaccgccttctatggaataaatcagttttcctatttgtttcctgaagagtttaaagccatttatttaagaagcaaaccttccaagtttcccagatactcagcagaagtacatatgtccatccccaatgtgtctttgccgttaagatttgactggagggacaagcaggttgtgacacaagtgagaaaccagcagatgtgtggaggatgctgggccttcagcgtggtgggggcagtggaatctgcttatgcaataaaggggaagcccctggaagacctaagtgtccagcaggtcattgactgttcgtataataattatggctgcaatggaggctctactctcaatgctttgaactggttaaacaagatgcaagtaaaactggtgaaagattcagaatatccttttaaagcacaaaatggtctgtgccattacttttctggttcacattctggattttcaatcaaaggttattctgcatatgacttcagtgaccaagaagatgaaatggcaaaagcacttcttacctttggccctttggtagtcatagtagatgcagtgagctggcaagattatctgggaggcattatacagcatcactgctctagtggagaagcaaatcatgcagttctcataactgggtttgataaaacaggaagcactccatattggattgtgcggaattcctggggaagttcttggggagtagatggttatgcccatgtcaaaatgggaagtaatgtttgtggtattgcagattccgtttcttctatatttgtgtgacatgttgggcagatcaagagacagctacaaaaatgaaggttttcataatgcaatgtaacatagtacttcaaagtattattcaacttcaagtttcagcaactacctacaaaagattctaaggcctagtagtatttaaactaagtttcagaatgttcccttcttgtagagagatggacaaccaaagtcagtgggacaaactccagcacagaagcctgcgaggaagcctatggaatagtttcctgtcctgagacgaaattcagattaggagatattttaggcccctgcaactggggaaggctactgtttgtttttgtttgcttattatttatttgtttgtttattgtgagatatttcaggtgggatcaaagaggtcataagaatttattttcttttgtggggtgtaactactagctttagattacccctatacacaagaatggccaacctaaaattatgtgtgtcttgtacagttagttatattagcagccctctgagatggcgtatctatcggaaggatttcaaacaccaattgctttacctgaacaaatggtgcttaccctttgaacagcagagtgaccacgtagaaggaaggaaaagggcaaaatcgcttcagttaaactgaaaccgaattc

SEQ ID NO: 133 - DNA, 1006 bp, Homo sapiens
gagaacgaaagatggcggcgcccgtaaggcggacgctgttaggggtggcggggggttggcggcggttcgagaggctctgggccggcagtctaagctctcgcagcctggctcttgcagccgcaccctcaagcaacggatccccatggcgcttgttgggcgcgttgtgcctgcagcggccacctgtagtctccaagccgttgaccccattgcaggaagagatggcgtctctactgcagcagattgagatagagagaagcctgtattcagaccacgagcttcgtgctctggatgaaaaccagcgactggcaaagaagaaagctgaccttcatgatgaagaagatgaacaggatatattgctggcgcaagatttggaagatatgtgggagcagaaatttctacagttcaaacttggagctcgcataacagaagctgatgaaaagaatgaccgaacatccctgaacaggaagctagacaggaaccttgtcctgttagtcagagagaagtttggagaccaggatgtttggatactgccccaggcagagtggcagcctggggagacccttcgaggaacagctgaacgaaccctggccacactctcagaaaacaacatggaagccaagttcctaggaaatgcaccctgtgggcactacacattcaagttcccccaggcaatgcggacagagagtaacctcggagccaaggtgttcttcttcaaagcactgctattaactggagacttttcccaggctgggaataagggccatcatgtgtgggtcactaaggatgagctgggtgactatttgaaaccaaaatacctggcccaagttaggaggtttgtttcagacctctgatgggccgagctgcctgtggacggtgctcagacaagtctgggattagagcctcaaggacattgtgtgattgcctcacatttgcaggtaatatcaagcagcaaactaaattctgagaaataaacgagtctattactgaaaaaaaaaaaaaaaaaaaa

SEQ ID NO: 134 - DNA, 3784 bp, Homo sapiens
ggcggaagcacgatctccggcagcggcctgggaactcttagctgagcaggcgagagcatcatggataccgacttatatgatgagtttgggaattatattggaccagagcttgattctgatgaagatgatgatgaattgggtagagagaccaaagatcttgatgagatggatgatgatgacgacgacgatgacgtaggagatcatgacgatgaccaccctgggatggaggtggtgctgcatgaggacaagaagtactacccaacagccgaggaggtgtatggtcctgaggtggagaccatagttcaagaggaagacactcagcctctcacagaacccattattaagccagtgaaaaccaagaaattcactctgatggagcagacattacctgttacggtgtatgagatggatttcttggcggatctgatggataactcagagctcatcagaaatgtgaccctttgtggacatctccaccatggcaagacatgttttgtggattgtttaattgaacagactcacccggaaatcagaaagcgctatgaccaagatctgtgctatactgacatcctcttcacagagcaagagagaggtgtaggcatcaaaagcactcctgtgacagtggtcttgccagacaccaaaggaaaatcttatctcttcaatatcatggacactccaggacatgtgaatttctctgatgaggtcacagctggcttgcgcatctcagatggagtggtccttttcattgatgctgctgagggggtgatgctgaacacagagcggctgatcaagcatgcggtgcaggagaggctggcagtcactgtgtgcatcaacaagattgaccggctgatcctggagctgaagctgcctccaactgatgcttattacaagctgcgccacattgtggatgaggtcaatggattaataagcatgtattccactgatgagaacctgatcctttccccactcctgggtaacgtctgcttctccagctcccagtacagcatctgcttcacgctgggctcctttgccaagatctatgccgacacctttggtgacattaattaccaagaatttgctaaaagactctggggtgacatctacttcaaccctaagacgcgaaagttcaccaaaaaggccccaactagcagctcccagagaagtttcgtggagtttatcttggagcctctttataagatcctcgcccaggttgtaggtgacgtggacaccagcctcccacggaccctagacgagcttggcatccacctgacgaaggaggagctgaagctgaacatccgccccttgctcaggctggtctgcaaaaagttctttggcgagttcacaggctttgtggacatgtgtgtgcagcatatcccttctccaaaggtgggcgccaagcccaagattgagcacacctacaccggtggtgtggactccgacctcggcgaggctatgagtgactgtgaccctgatggccccctgatgtgccacactactaagatgtacagcacagatgatggagtccagtttcacgcctttggccgggtgctgagtggcaccattcatgctgggcagcctgtgaaggtactgggggagaactacaccctggaggatgaggaagactcccagatatgcaccgtgggccgcctttggatctctgtggccaggtaccacatcgaggtgaaccgtgttcctgctggcaactgggttctgattgaaggtgttgatcaaccaattgtgaagacagcaaccataaccgaaccccgaggcaatgaggaggctcagattttccgacccttgaagttcaataccacatctgttatcaagattgctgtggagccagtcaacccctcagagctgcccaagatgcttgatggcctgcgcaaggtcaacaagagctatccatccctcaccaccaaggtggaggagtctggcgagcatgtgatcctgggcactggggagctctacctggactgtgtgatgcatgatttgcggaagatgtactcagagatagacatcaaggtggctgacccagttgtcacgttttgtgagacggtggtggaaacatcctccctcaagtgctttgctgaaacgcctaataagaagaacaagatcaccatgattgctgagcctcttgagaagggcctggcagaggacatagagaatgaggtggtccagattacgtggaacaggaagaagctgggagagttcttccagaccaagtacgattgggatctgctggctgcccgttccatctgggcttttggccctgatgcgactggccccaacattctggtggatgatactctgccctctgaggtggacaaggctcttcttggttcagtgaaggacagcatcgttcaaggtttccagtggggaaccagggagggccccctctgtgatgaattgattcggaatgtcaagtttaagatcctggatgcggtggttgcccaggagcccctgcaccggggcgggggccagatcatccccacagccaggagagtcgtctactctgccttcctcatggctactcctcgtctgatggagccttactactttgtagaggtccaggcccctgcagattgcgtctctgcagtttataccgtcctggccaggcgcagggggcacgtgactcaggatgcacccatcccaggctcccctctgtacaccatcaaagcttttatcccggccatcgactcttttggctttgagactgatctccggactcacacccagggacaagccttttctctgtctgtcttccaccactggcagattgtgcctggtgatcccctggacaagagcattgtcatccgccccttggagccacagccagctcctcacctggcccgggaattcatgatcaaaacccgccgtaggaagggcctcagtgaagatgtgagcatcagcaaattcttcgatgatcctatgttgctggaacttgccaaacaggatgttgtgctcaattaccccatgtgagtgcgtggactcctgggagctcctgctccctacagtgggctgcaactcctgtacttgaagctgagacctcatatgacgtggccttcgtgttgtcagagagtgtctggaagctgctgttgccatcttgaacaactcaccaacctccaacccagagccccagtgagagaggagcatttggcctcctgcttccttctgtggcctctgccgggctccattcccaaggaaaagagaggagcttgggctcacagaaagagaaggggatgaaaccccaaggggccctatctttgggatttacatggaattttattttctacaagtttgaccttagccatggtttgcaagtgaacagaacattctgacctctgtcttgctctgctcctttcatcctcgtctcccctgccccatctggtgcttacattctgaatatatgtcatctcccaagaggcttcactgcctctgcttccagctgcagcctccttcctgcctgggtccccagggaagccgcctgccttttaattcagtgttcccatgagcgccaaggccccattattgcccccttgctcccactccatgctgcttctgggtgggacctaagatggcttgggagttgttgggttcctgcgatcagaagtctaccccaccacctcctcaggaaactgctgcctcccctaagaatcttccttgccctggagtagggggccagagcactttgatttccagccatttactccaagtcctctccccagctaccaccagtcccttactctgttctcccccagtgaaaaagagtctgttgattttcctc

SEQ ID NO: 135 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 136 - DNA, 246 bp, Homo sapiens
gcaccctgcaatatctgagccaggggaacgtggtgttcaaggactccgtgaaggtcatgacagtgaattacaacacgcatggggagctgggcgagggcgccaggtcagtcgcagccccggccgcctggcgagccgtaacctgctagtcgtgccagcctgctggtaacgtgaggtcccgagtcgctctgggcggccgttcatccccggggcgggccccccaggtcggcgctgttacccccattttac

SEQ ID NO: 137 - DNA, 1473 bp, Homo sapiens
attagcgcctgcgccgtctctaggccccgccccctcacccctccggtcctggagctcccacagctaacatggcggcgccctgtgtgtcctacggcggagcagtttcgtaccggcttcttctctggggtaggggtagcctcgcccggaagcaaggcctctggaaaaccgcggcccctgagttgcaaacaaatgtcagatcccagatattaaggctaagacatactgcatttgtaataccaaagaaaaacgttcctacctcaaaacgtgaaacttacacagaggattttattaaaaagcagattgaagagttcaacataggaaagagacatttagccaacatgatgggagaagatccagaaactttcactcaagaagatattgacagagctattgcttaccttttcccaagtggtttgtttgagaaacgagccaggccagtaatgaagcatcctgaacagatttttccaagacaaagagcaatccagtggggagaagatggccgtccatttcactatctcttctatactggcaaacagtcatactattcattaatgcatgatgtatatggaatgttactcaatttagaaaaacatcaaagtcacttgcaagccaaaagtctgctcccagaaaaaactgtaaccagagacgtgattggcagcagatggctgattaaggaggaactagaagaaatgttagtggaaaaactgtcagatctagattatatgcagttcattcggctgctagaaaagttattgacatcgcagtgtggtgctgctgaggaagaatttgtgcagaggtttcgaagaagtgtaactcttgaatcaaaaaaacagctgattgaacctgtacagtatgatgagcaaggaatggcctttagcaaaagtgaaggtaaaagaaagactgcaaaagcagaagcaattgtttataaacatggaagtggaagaataaaagtaaatggaattgattaccagctttacttcccgatcacacaggacagagaacagctgatgttccctttccactttgttgaccggctgggaaagcacgacgtgacctgcacagtctcagggggcgggaggtcagcgcaggctggagcaatacgactggcaatggcaaaagccttgtgcagctttgtcaccgaggacgaggtcgagtggatgagacaagctggactacttactactgatccacgtgtgagggaacggaagaagccaggccaagagggagcccgcagaaagtttacgtggaagaaacgctaagggtttgctcccaggaaaggagaggaagagctatatatatgtgccgacatgtggcagacacacagtaaataatggctgaccagcatgagggcagtactgtcagaaatttctttgagctgtgagatggatttatttttaaatgctactttgtaaaggtgacctttaaaaaataaaaggaaaataaagaatgttagtttcaaaaaaaaaaaaaaaa

SEQ ID NO: 138 - DNA, 2213 bp, Homo sapiens
atacttgaaaaacattacaattcgtagtgtttttacaaaaagatcttcatgtttcaaatttagacagcatcaccgtctaccataagggatgaagacattggcactctcacaaatgatataattttcttccacctttaccaatttatgttgggtaactttctcactcaaagtttataacatttatagtctgtttagtaaccatatctgcacaacctctactgttcattttatgttgaaccagataagatgcttaccaccaatctttttcctgatatatggcttaattggctgtatttaagaaattatgacttctcagaaaaagtaatcctagaaatatcaaaaggaggcttaatctttagaaaattaccatgtttagaaataagactgagtgcagtggctcacacctataatcccagcactttgggaggctagggcaggatgattgcttgaacccaggagtttgagaccagcctggacaacaaagctttccagagtgttgaatcttccaccagaaaacttgatcacatcaatatctgcagttccaatttcccaaaaagaagaagtagctgattttcagctttctgtggattctttattggaaaaagacaatgaccattcaagaccagatattcaagttcaagccaagagactagcagagaagctaagatgtgatacagtggtgagtgaaatcagtactggtcaaaggactgtaaatttcaaaataaacagagagctcttaacaaagacagtgctacaacaagtaattgaagatggctcaaaatatggattaaaaagtgaacttttctctggacttccccagaagaagattgtggttgaattcagttcacctaatgttgccaaaaaatttcatgttggacatttgcgttctaccatcataggaaattttatagcaaatctcaaagaagctttaggacatcaagtaataagaataaattaccttggcgattggggcatgcagtttggtcttctgggaactggcttccagctgtttggctatgaggaaaaactgcagtccaatcctctacagcatctctttgaagtttatgtacaagttaataaagaagcagcagatgataaaagtgtagcaaaagcagcacaggagttcttccaacgattggaactgggcgatgtgcaagcactttcactgtggcaaaaatttcgggacttgagcattgaagagtacattcgggtttacaagcgtctgggagtatattttgatgaatattcaggagaatcattttatcgtgaaaaatctcaagaggtcttaaagttgctggagagtaaaggactcctactgaaaacaataaaaggaacggctgtagtagatctctctgggaatggcgacccctcctcaatttgtactgtaatgcgaagtgatgggacttctctctatgcaaccagagatcttgcagctgctatagatcgaatggacaagtataattttgatacaatgatatatgtgacagataaaggacaaaaaaagcattttcagcaagtattccaaatgctgaagatcatgggatatgactgggcagaaaggtgccagcacgtgccctttggagtagtacagggaatgaagactcgaggaggagatgtcactttcctggaagatgttttaaatgagattcaattaaggatgctacagaacatggcttcaattaagacaactaaagaactcaagaacccacaagagactgcagagagggtcgggctcgcagcactcattattcaggacttcaaaggtttactcttatctgactacaagttcagctgggatcgtgttttccagagtcgcggggacacaggagtcttcctacagtacacacacgcccgcctccacagtttggaagagacttttggatgtgggtacctgaatgacttcaacactgcttgtttacaagagccacagtctgtttcaattcttcagcatcttctcaggttcgacgaggtgctttataaatcatctcaggactttcaacccaggcatatcgtcagttaccttctaactttaagtcatcttgcagctgtggcacacaaaacactacaaataaaagatagtcctcctgaagtggctggggccagacttcatcttttcaaagctgtccgttctgtcctagccaatggaatgaaacttcttggaataacacctgtatgtaggatgtaatttccattaaaatggcttttaaaatgtc

SEQ ID NO: 139 - DNA, 1161 bp, Homo sapiens
agacaccgtgtcctcttgcctgggagaggggaagcagatctgaggacatctctgtgccaggccagaaaccgcccacctgcaggtgaggcccggacccctgcccagttccttctccgggatggacgtggggcccagctccctgccccaccttgggctgaggctgctgctgctcctgctgctgctgcccctcaggggccaagccaacacaggctgctacgggatcccagggatgcccggcctgcctggggcaccagggaaggatgggtacgacggactgccggggcccaagggggagccaggaatcccagccattcccgggatccgaggacccaaagggcagaagggagaacccggcttacccggccatcctgggaaaaatggccccatgggaccccctgggatgccaggggtgcccggccccatgggcatccctggagagccaggtgaggagggcagatacaagcagaaattccagtcagtgttcacggtcactcggcagacccaccagccccctgcacccaacagcctgatcagattcaacgcggtcctcaccaacccgcagggagattatgacacgagcactggcaagttcacctgcaaagtccccggcctctactactttgtctaccacgcgtcgcatacagccaacctgtgcgtgctgctgtaccgcagcggcgtcaaagtggtcaccttctgtggccacacgtccaaaaccaatcaggtcaactcgggcggtgtgctgctgaggttgcaggtgggcggggaggtgtggctggctgtcaatgactactacgacatggtgggcatccagggctctgacagcgtcttctccggcttcctgctcttccccgactagggcgggcagatgcgctcgagacccacgggccttccacctccctcagcttcctgcatggacccaccttactggccagtctgcatccttgcctagaccattctcccctccagggagcccaccctgacccacccccactgcaccccctccccatgggttctctccttcctctgaacttctttaggagtcactgcttgtgtggttcctgggacacttaaccaatgccttctggtactgccattcttttttttttttcaagtattggaaggggtggggagatatataaataaatcatgaaatcaatacat

SEQ ID NO: 140 - DNA, 2472 bp, Mus musculus
tctgtgttgctttgagcctgatctcctgtgctggcagaagtctctcagttggtgtgggcgatgctgtggcaggagatcgcggtggactgttcttcctgggcactgtcatcttgggtgaggaagctaaggtagagtgagtcaaggacggcctgggaaggactgccttgggctgttttcattttccaagtggattttgttgattggaatacagattgaaaacttgaaaggataatgctttgcctcctgaaactgattgtgattccagtaatcctggcccctgtaggttatccacagggcctgccaggcttgaccgtttcctcccctcagctgagagtgcatgtgggtgaatcagtcttgatgggatgtgttgtccagcgcacagaagagaaacacgtggacagagtggattggctcttctcgaaagataaagatgatgcgagtgaatatgtgctgttctactattccaaccttagcgtgcctacggggcgcttccagaaccggtcacatttggtgggggacaccttccataatgatggttctctcctgctccaagatgttcagaaggccgatgagggaatctacacctgtgaaatccgcctcaaaaatgagagcatggtgatgaaaaagcccgtggaactgtgggtgctaccagaggaacctaaagatctcagagtccgagtaggtgatacaactcagatgagatgttctatccagagcacagaagagaaacgggtgaccaaagtaaactggatgttttcttcaggaagccatactgaggaggagacagtcttgagctatgactccaacatgcgtagtggaaaattccagagcctgggccgcttccgcaaccgtgtagacctgacaggtgacatctcccgcaatgatggctcaatcaaacttcaaacagtgaaggagtctgaccaaggaatctacacttgcagcatctacgtgggaaagctggagtccaggaaaaccattgtgctgcatgtggtccaggacgaatttcaaaggacaatttcaccaactcctccaactgataagggtcagcagggcatcctgaatggaaatcagctggtgatcattgtggggatcgtctgtgccaccttcctgctgcttccggttttgatattaattgtgaagaaagccaagtggaataagagctcagtaagttctatggcttctgtgaagagcctggagaacaaagagaagattaatccagagaagcatatctactcctccataactacgtgggagacgacagagagaggaataagtggagagtcagagggcacctacatgaccatgaatccagtttggccttcttcccccaaagcatccagtttggtccgctcttcagtcagatccaagtaatgtggttgaaaagaaaaaaaaaaaaagtcaccttggggaatttccaaaaccgagcaaactttctgagaaggaggagagactctttttgatggtccctgggactactgtgccagctattttagacttgtgcattccatctggtcttggcctccatcaaaaggctgaagacggagggtttggagtctggcaggaaggctgaactgctccggataaccaggcactgatgaggggagggagacctcggacttctcccagaagtggtgtgcgggctggtgaggatggggagcagcctggtctgccttgtgctttcaagctctccattggactggatctgtaacgtgaacagtggtttgcttgtttgttgttcgtttttgcggatgagctactaagagacacacagagatataaaccaaaaaaaaaaaaaattattttgatgaattatccccaaataggtgtgatttgttggaatcatctttttttatgtcccagatgaaggacaagtggggaaatttgaagcatggaactcctgagagagtgttctttgaggagagccacaatggactcagcatctggggactggtttggatctggggggaagacttaggggtaacccattggaagtgggatatgagaacagctctgggatttcctcgggctgggaatctgcactgatggctgaagctaggcctgtggagtttggacatccatgttttccaaatgcattgaacaggcttgccaaagcgaacagccaacgattgaatccttggtctgaaatatgagactcctgtacttttcattgttatcatgagacttaaacaggttgatcttatttaaatattttttttttaaatgggagtggtgctggagagatagcccatcagttaagagaactgactgctcttgcagaagactggggtttgatgcccagcatccacatggtggctcataactgtaactccaattccaagggagctgacaccttcttctggcctttgtggtccctgcatgtgcaaggtacatggacatacatacatatatataaaagttttttaaaaatggtcaataaaaactaaaagtgttgatcttaaaaaaaaaaaaaaa

SEQ ID NO: 141 - DNA, 593 bp, Homo sapiens
gaggcggggccagcgaggcaagatggagttagtgcaggtcctgaaacgcgggctgcagcagatcaccggccacggcggtctccgaggctatctacgggtttttttcaggacaaatgatgcgaaggttggtacattagtgggggaagacaaatatggaaacaaatactatgaagacaacaagcaattttttggccgtcaccgatgggttgtatatactactgaaatgaatggcaaaaacacattctgggatgtggatggaagcatggtgcctcctgaatggcatcgttggcttcacagtatgactgatgatcctccaacaacaaaaccacttgctgctcgtaaattcatttggacgaaccataaattcaacgtgactggcaccccagaacaatatgtaccttattctaccactagaaagaagattcaggagtggatcccaccttcaacaccttacaagtaaagacaatgaagaacagttgaaacatgcaaaatatggagcttttcatgtaattactcttttactgtttaccattcactataattcacaattaaaattgtgtgaccaaaaaaaaaaaaaaaaaaaaaaaaaaaaa